(19) United States
(12) Patent Application Publication
(10) Pub. No.: US 2025/0304989 A1
(43) Pub. Date: Oct. 2, 2025

(54) METHODS OF INCREASING PLANT PRODUCTIVITY AND TOLERANCE TO WATER & NUTRIENT DEFICIENCY

(71) Applicant: Performance Plants Inc., Kingston (CA)

(72) Inventors: Jiangxin WAN, Bath (CA); Shujun YANG, Kingston (CA); Kate JOSLIN, Verona (CA); Xurong TANG, Kingston (CA); Gang TIAN, Kingston (CA); Yafan HUANG, Bath (CA)

(21) Appl. No.: 18/579,239

(22) PCT Filed: Jul. 13, 2022
(86) PCT No.: PCT/CA2022/051091

§ 371 Date: Jan. 12, 2024

Related U.S. Application Data
(60) Provisional application No. 63/222,193, filed on Jul. 15, 2021.

(51) Int. Cl. C12N 15/82; C12N 9/22; C12N 15/11

(52) U.S. Cl. CPC C12N 15/8273; C12N 9/226; C12N 15/11; C12N 15/8213; C12N 15/8271; C12N 2310/20

(57) ABSTRACT

The present invention provides methods of increasing plant productivity and tolerance to water and nutrient deficiency by overexpressing an exonuclease responsible for the d200 mutant. Also provided are plants which overexpress the exonuclease and have improved tolerance to water and nutrient deficiency.

### CROSS REFERENCE TO RELATED APPLICATIONS

This application is a U.S. National Phase application, filed under 35 U.S.C. § 371(c), of International Application No. PCT/CA2022/051091, filed Jul. 13, 2022, which claims benefit of U.S. Provisional Application No. 63/222,193, filed Jul. 15, 2021, the contents of each of which are hereby incorporated by reference in their entirety.

### REFERENCE TO AN ELECTRONIC SEQUENCE LISTING

The contents of the electronic sequence listing (PREP-020_N01US_SeqListing_st26.xml; Size: 896,437 bytes; and Date of Creation: Dec. 18, 2024) are herein incorporated by reference in its entirety.

### FIELD OF INVENTION

The present invention relates to methods of increasing tolerance to water and nutrient stresses and improvement of plant water use efficiency, and methods of increasing yield including root, shoot and seed production of a plant, plant part or plant cell under various environmental conditions.

### BACKGROUND OF THE INVENTION

Plants are often subject to various environmental stresses such as drought, high temperature, cold and excess salt throughout their development (Zhu 2016). Drought as a major environmental factor may adversely affect various aspects of plant development including seed germination, vegetative growth, fertility and seed filling, thus limiting plant productivity in agriculture. Plants respond to drought via complex regulatory networks starting from water deficit sensing to various molecular, cellular, and physiological responses (Yang et al, 2010; Takahashi et al., 2018).

As some examples, drought tolerance could be improved by modulating stomatal density (Yoo et al., 2010) or stomatal transpiration regulated by phytohormone abscisic acid (ABA, Mega et al., 2019; Yang et al., 2019). Drought tolerance could also be improved by stabilizing active conformation of cellular proteins or RNA molecules under stressed conditions. For instance, ectopic expression of bacterial RNA chaperones in corn confers plant drought tolerance and higher grain yield under water-limited field conditions (Castiglioni et al., 2008). Plant transcription complex such as nuclear factor Y (NF-Y) and Hardy (HRD) could act as regulators for various physiological responses. Over-expression of NF-Y or HRD in corn or wheat makes the transgenic crops more tolerant to drought under water-limited field conditions respectively (Nelson et al., 2007; Karaba et al., 2007).

The discovery of these regulators of plant response to water deficiency facilitate the development of biotechnologies for enhancing drought tolerance in crop plants. However, the successful application of the technologies in the field is still scarce. As current agricultural crops bred for yield have generally less resources or morphological capacity to withstand long periods of intense water deficit, it is critical that these crops are able to adapt to water shortage by improving root growth to reach more water resources.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide methods of increasing plant productivity and tolerance to water and nutrient deficiency. In accordance with an aspect of the present invention, there is provided a method of increasing tolerance to water and/or nutrient deficiency in a plant, comprising: modifying expression or activity of AtExo970, homolog or ortholog thereof.

In certain embodiments, the method comprises a) introducing a nucleic acid construct to a plant, a plant tissue culture or a plant cell to obtain a modified plant, a modified plant tissue culture or a modified plant cell, wherein the nucleic acid construct encodes the AtExo970, homolog or ortholog thereof; b) growing the modified plant or regenerating a plant from the modified plant tissue culture or the modified plant cell; and c) selecting a plant having increased tolerance to water and/or nutrient deficiency relative to a wild type plant. In certain embodiments, the method comprises a) introducing one or more nucleic acid constructs for CRISPR mediated replacement of the native promoter of the gene for AtExo970, homolog or ortholog to a plant, a plant tissue culture or a plant cell to obtain a modified plant, a modified plant tissue culture or a modified plant cell; b) growing the modified plant or regenerating a plant from the modified plant tissue culture or the modified plant cell; and c) selecting a plant having increased tolerance to water and/or nutrient deficiency relative to a wild type plant.

In accordance with another aspect of the present invention, there is provided a method of increasing plant productivity, comprising: modifying expression or activity of AtExo970, homolog or ortholog thereof.

In certain embodiments, the method comprises a) introducing a nucleic acid construct to a plant, a plant tissue culture or a plant cell to obtain a modified plant, a modified plant tissue culture or a modified plant cell, wherein the nucleic acid construct encodes the AtExo970, homolog or ortholog thereof; b) growing the modified plant or regenerating a plant from the modified plant tissue culture or the modified plant cell; and c) selecting a plant having increased plant productivity relative to a wild type plant.

In certain embodiments, the method comprises a) introducing one or more nucleic acid constructs for CRISPR mediated replacement of the native promoter of the gene for AtExo970, homolog or ortholog to a plant, a plant tissue culture or a plant cell to obtain a modified plant, a modified plant tissue culture or a modified plant cell; b) growing the modified plant or regenerating a plant from the modified plant tissue culture or the modified plant cell; and c) selecting a plant having increased tolerance to water and/or nutrient deficiency relative to a wild type plant.

### DETAILED DESCRIPTION OF THE INVENTION

This invention starts from the identification and characterization of an Arabidopsis mutant d200 from an Activation-tag population (Weigel et al, 2000). d200 showed reduced water loss through transpiration, reduced flower abortion, improved pollen viability under limited water conditions, and increased root and shoot growth under optimal as well as water and nutrient deficit conditions, ultimately enhanced drought tolerance, water use efficiency and plant productivity compared to the parent plant. Gene AtExo970 (TAIR ID At3g27970) was identified as being responsible for the observed phenotypes in d200 mutant. The endogenous AtExo970 has an extremely low basal expression in leaves, stems and flowers in wildtype Arabidopsis, but is highly up regulated in d200 mutant due to the presence of expression enhancer tag located close to the AtExo970 locus. AtExo970 encodes for RNA exonuclease and may be involved in ribosomal RNA (rRNA) or ribosome biogenesis and processing, that ultimately affect the functionality of genes required for plant drought tolerance. Ectopic over-expression of AtExo970 or its orthologs from either monocots (such as wheat, rice, maize and et al) or dicots species (such as canola, soybean, cotton and et al) under constitutive promoter was able to mimic the phenotypes of d200 mutant in transgenic Arabidopsis, soybean and Brachypodium.

### Nucleic Acids and Polypeptides:

A genetic screen was used to identify a novel exonuclease, AtExo970, and subsequently its orthologs from various plant species which improve tolerance to water and nutrient deficiency as well as improve plant productivity mainly by increasing root growth especially under stressed conditions.

Accordingly, the present invention provides nucleic acids encoding AtExo970, homologs, orthologs, variants and fragments thereof. The nucleic acid includes DNA, such as cDNA or genomic DNA, or RNA such as mRNA.

In certain embodiments, there is provided a nucleic acid comprising the sequence as set forth in any one of the sequences set forth herein encoding AtExo970 homologs, orthologs, variants and fragments thereof. In specific embodiments, the sequence comprises the sequence as set forth in any one of SEQ ID NOs: 122, 123, 126, 127, 128, 129, 131, 132, 134, 135, 137, 138, 140, 141, 143, 144, 146, 147, 149, 150, 152, 153, 155, 156, 158, 159, 161, 162, 164, 165, 167, 168, 170, 171, 173, 174, 175, 177, 178, 180, 181, 182, 184, 185, 187, 188, 190, 191, 192, 194, 195, 196, 198, 200, 201, 203, 205, 206, 208, 209, 212, 213, 215, 216, 218, 219, 221, 222, 224, 225, 227, 228, 230, 231, 233, 234, 236, 237, 239, 240, 242, 243, 245, 246, 248, 249, 251, 252, 254, 255, 257, 258, 260, 261, 263, 264, 266, 267, 269, 270, 272, 273, 275, 276, 278, 279, 281, 282, 284, 285, 287, 288, 290, 291, 293, 294, 296, 297, 299, 300, 302, 303, 305, 306, 308, 309, 311, 312, 314, 315, 317, 318, 320, 321, 323, 324, 326, 327, 329, 330, 332, 333, 335, 336, 338, 339, 341, 342, 343, 344, 345, 347, 348, 350, 351, 353, 354, 356, 357, 359, 360, 362, 363, 365, 366, 368, 370, 371, 373, 374, 376, 377, 379, 380, 382, 383, 385, 386, 388, 389, 391, 392, 394, 395, 397, 398, 400, 401, 403, 404, 406, 407, 409 and 410.

In certain embodiments, there is provided a nucleic acid or encoding the sequence of any one of SEQ ID NOs: 124, 125, 130, 133, 136, 139, 142, 145, 148, 151, 154, 157, 160, 163, 166, 169, 172, 176, 179, 183, 186, 189, 193, 197, 199, 202, 204, 207, 211, 214, 217, 220, 223, 226, 229, 232, 235, 238, 241, 244, 247, 250, 253, 256, 259, 262, 265, 268, 269, 270, 271, 272, 273, 274, 275, 276, 277, 278, 279, 280, 283, 286, 289, 292, 295, 298, 301, 304, 307, 310, 313, 316, 319, 322,325, 328, 331, 334, 337, 340, 343, 346, 349, 352, 355, 358, 361, 364, 367, 369, 372, 375, 378, 381, 384, 387, 390, 393, 396, 399, 402, 405 and 408.

In some embodiments of the present invention, there is provided a nucleic acid comprising any one of the sequences set forth above comprising one or more substitutions, insertions and/or deletions. Such nucleotide sequences may or may not encode a protein having the same biological activity as the protein comprising reference sequence. Expression of nucleic acids encoding a protein that is not fully functional can be useful in a dominant/negative inhibition method.

In other embodiments, there is provided a nucleic acid comprising a sequence having at least 50%, 55%, 60%, 65%, 70%, 75%, 80%, 85%, 90%, 95%, 96%, 97%, 98%, 99% or 100% identity to any one of the sequences set forth in SEQ ID NOs: 122, 123, 126, 127, 128, 129, 131, 132, 134, 135, 137, 138, 140, 141, 143, 144, 146, 147, 149, 150, 152, 153, 155, 156, 158, 159, 161, 162, 164, 165, 167, 168, 170, 171, 173, 174, 175, 177, 178, 180, 181, 182, 184, 185, 187, 188, 190, 191, 192, 194, 195, 196, 198, 200, 201, 203, 205, 206, 208, 209, 212, 213, 215, 216, 218, 219, 221, 222, 224, 225, 227, 228, 230, 231, 233, 234, 236, 237, 239, 240, 242, 243, 245, 246, 248, 249, 251, 252, 254, 255, 257, 258, 260, 261, 263, 264, 266, 267, 269, 270, 272, 273, 275, 276, 278, 279, 281, 282, 284, 285, 287, 288, 290, 291, 293, 294, 296, 297, 299, 300, 302, 303, 305, 306, 308, 309, 311, 312, 314, 315, 317, 318, 320, 321, 323, 324, 326, 327, 329, 330, 332, 333, 335, 336, 338, 339, 341, 342, 343, 344, 345, 347, 348, 350, 351, 353, 354, 356, 357, 359, 360, 362, 363, 365, 366, 368, 370, 371, 373, 374, 376, 377, 379, 380, 382, 383, 385, 386, 388, 389, 391, 392, 394, 395, 397, 398, 400, 401, 403, 404, 406, 407, 409 and 410, and fragments thereof. In certain embodiments, fragments are at least 10, at least 20, at least 50 nucleotides in length. The fragments may be used, for example, as primers or probes.

In other embodiments, there is provided a nucleic acid encoding a polypeptide comprising a sequence at least 50%, 55%, 60%, 65%, 70%, 75%, 80%, 85%, 90%, 95%, 96%, 97%, 98%, 99% (or more) percent identity to any one of the sequences set forth in SEQ ID NOs: 124, 125, 130, 133, 136, 139, 142, 145, 148, 151, 154, 157, 160, 163, 166, 169, 172, 176, 179, 183, 186, 189, 193, 197, 199, 202, 204, 207, 211, 214, 217, 220, 223, 226, 229, 232, 235, 238, 241, 244, 247, 250, 253, 256, 259, 262, 265, 268, 269, 270, 271, 272, 273, 274, 275, 276, 277, 278, 279, 280, 283, 286, 289, 292, 295, 298, 301, 304, 307, 310, 313, 316, 319, 322,325, 328, 331, 334, 337, 340, 343, 346, 349, 352, 355, 358, 361, 364, 367, 369, 372, 375, 378, 381, 384, 387, 390, 393, 396, 399, 402, 405 and 408 and fragments thereof.

In certain embodiments, there are provided nucleic acids further comprise or encode heterologous sequences. The heterologous sequences may include but are not limited to markers, including fluorescent markers such as GFP, herbicide and/or pest resistance proteins such as EPSPS. In certain embodiments, the present invention provides nucleic acids encoding the polypeptide of the invention with herbicide and/or pest resistance proteins. In specific embodiments, the present invention provides nucleic acids comprising any of the sequences set forth above together with sequences encoding EPSPS, GPR or GFR. In specific embodiments, the present invention provides nucleic acids comprising any one of the sequences set forth above together with sequences encoding Cry1Ac, Cry1Ca and Cry3Aa. In certain embodiments, the present invention provides nucleic acids encoding fusion proteins comprising the polypeptide of the present invention and a heterologous polypeptide. In certain embodiments, the fusion polypeptide comprises a linker sequence between the polypeptides.

Also provided are nucleic acids that hybridize to the nucleic acids of the present invention. In certain embodiments, there is provided a nucleic acid that hybridizes to any one of the sequences as set forth in SEQ ID NOs: 122, 123, 126, 127, 128, 129, 131, 132, 134, 135, 137, 138, 140, 141, 143, 144, 146, 147, 149, 150, 152, 153, 155, 156, 158, 159, 161, 162, 164, 165, 167, 168, 170, 171, 173, 174, 175, 177, 178, 180, 181, 182, 184, 185, 187, 188, 190, 191, 192, 194, 195, 196, 198, 200, 201, 203, 205, 206, 208, 209, 212, 213, 215, 216, 218, 219, 221, 222, 224, 225, 227, 228, 230, 231, 233, 234, 236, 237, 239, 240, 242, 243, 245, 246, 248, 249, 251, 252, 254, 255, 257, 258, 260, 261, 263, 264, 266, 267, 269, 270, 272, 273, 275, 276, 278, 279, 281, 282, 284, 285, 287, 288, 290, 291, 293, 294, 296, 297, 299, 300, 302, 303, 305, 306, 308, 309, 311, 312, 314, 315, 317, 318, 320, 321, 323, 324, 326, 327, 329, 330, 332, 333, 335, 336, 338, 339, 341, 342, 343, 344, 345, 347, 348, 350, 351, 353, 354, 356, 357, 359, 360, 362, 363, 365, 366, 368, 370, 371, 373, 374, 376, 377, 379, 380, 382, 383, 385, 386, 388, 389, 391, 392, 394, 395, 397, 398, 400, 401, 403, 404, 406, 407, 409 and 410 under conditions of low, moderate or high stringency. A worker skilled in the art readily appreciates that hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. Such a worker could readily determine appropriate stringent (see, for example, Sambrook, et al., Molecular Cloning: A Laboratory Manual, 2nd ed., Cold Spring Harbor Laboratory Press, New York (1989) pp. 9.50-51, 11.48-49 and 11.2-11.3).

Typically under high stringency conditions only highly similar sequences will hybridize (typically >95% identity). Under moderate stringency conditions typically those sequence having greater than 80% identity will hybridize and under low stringency conditions those sequences having greater than 50% identity will hybridize.

A non-limiting example of “high stringency conditions” when used in reference to nucleic acid hybridization comprise conditions equivalent to binding or hybridization at 42° C. in a solution consisting of 5×SSPE (43.8 g/I NaCl, 6.9 g/I NaH2PO4H2O and 1.85 g/I EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed. A non-limiting example of “medium stringency conditions” when used in reference to nucleic acid hybridization comprise conditions equivalent to binding or hybridization at 42° C. in a solution consisting of 5×SSPE (43.8 g/I NaCl, 6.9 g/I NaH2PO4H2O and 1.85 g/I EDTA, pH adjusted to 7.4 with NaOH), 0.5% SDS, 5×Denhardt's reagent and 100 μg/ml denatured salmon sperm DNA followed by washing in a solution comprising 1.0XSSPE, 1.0% SDS at 42° C. when a probe of about 500 nucleotides in length is employed. A non-limiting example “Low stringency conditions” when used in reference to nucleic acid hybridization comprise conditions equivalent to binding or hybridization at 42° C. in a solution consisting of 5×SSPE (43.8 g/I NaCl, 6.9 g/I NaH2PO4H2O and 1.85 g/I EDTA, pH adjusted to 7.4 with NaOH), 0.5% SDS, 5×Denhardt's reagent and 100 μg/ml denatured salmon sperm DNA followed by washing in a solution comprising 5×SSPE, 0.1% SDS at 42° C. when a probe of about 500 nucleotides in length is employed.

Also provided are nucleic acids that are complementary to the nucleic acids of the present invention. In certain embodiments, there is provided a nucleic acid that hybridizes to any one of the sequences as set forth in SEQ ID NOs: SEQ ID NOs: 122, 123, 126, 127, 128, 129, 131, 132, 134, 135, 137, 138, 140, 141, 143, 144, 146, 147, 149, 150, 152, 153, 155, 156, 158, 159, 161, 162, 164, 165, 167, 168, 170, 171, 173, 174, 175, 177, 178, 180, 181, 182, 184, 185, 187, 188, 190, 191, 192, 194, 195, 196, 198, 200, 201, 203, 205, 206, 208, 209, 212, 213, 215, 216, 218, 219, 221, 222, 224, 225, 227, 228, 230, 231, 233, 234, 236, 237, 239, 240, 242, 243, 245, 246, 248, 249, 251, 252, 254, 255, 257, 258, 260, 261, 263, 264, 266, 267, 269, 270, 272, 273, 275, 276, 278, 279, 281, 282, 284, 285, 287, 288, 290, 291, 293, 294, 296, 297, 299, 300, 302, 303, 305, 306, 308, 309, 311, 312, 314, 315, 317, 318, 320, 321, 323, 324, 326, 327, 329, 330, 332, 333, 335, 336, 338, 339, 341, 342, 343, 344, 345, 347, 348, 350, 351, 353, 354, 356, 357, 359, 360, 362, 363, 365, 366, 368, 370, 371, 373, 374, 376, 377, 379, 380, 382, 383, 385, 386, 388, 389, 391, 392, 394, 395, 397, 398, 400, 401, 403, 404, 406, 407, 409, and 410 or fragment thereof.

A worker skilled in the art would readily appreciate that CRISPR methodologies may be used for targeted DNA alteration in plant cells. In such methodologies a CRISPR-Cas system guide RNA that hybridizes with the target sequence is utilized. Accordingly, the present invention also provides nucleic acids that hybridizes to target sequences to modify endogenous expression of exonuclease of the present invention. Exemplary guide nucleic acids for use in CRISPR methodologies include but are not limited to SEQ ID NOs: 68, 69, 70, 71 and 72.

In specific embodiments, CRISPR is utilized to replace the native promoter of the exonuclease gene of the present invention. In such embodiments, there is provided a HDR template containing the new promoter. The promoter may be a constitutive promoter, an inducible promoter, or tissue specific promoter. Non-limiting examples of promoters are set forth in SEQ ID NOs: 414, 415, 426, 427, 452, 453, 454, 455, 456, 457, 458, 459, 460 and 461.

The present invention also provides AtExo970, homologs, orthologs, variants and fragments thereof.

In certain embodiments, there is provided a polypeptide comprising a sequence encoded by the sequence as set forth in any one of SEQ ID NOs: 122, 123, 126, 127, 128, 129, 131, 132, 134, 135, 137, 138, 140, 141, 143, 144, 146, 147, 149, 150, 152, 153, 155, 156, 158, 159, 161, 162, 164, 165, 167, 168, 170, 171, 173, 174, 175, 177, 178, 180, 181, 182, 184, 185, 187, 188, 190, 191, 192, 194, 195, 196, 198, 200, 201, 203, 205, 206, 208, 209, 212, 213, 215, 216, 218, 219, 221, 222, 224, 225, 227, 228, 230, 231, 233, 234, 236, 237, 239, 240, 242, 243, 245, 246, 248, 249, 251, 252, 254, 255, 257, 258, 260, 261, 263, 264, 266, 267, 269, 270, 272, 273, 275, 276, 278, 279, 281, 282, 284, 285, 287, 288, 290, 291, 293, 294, 296, 297, 299, 300, 302, 303, 305, 306, 308, 309, 311, 312, 314, 315, 317, 318, 320, 321, 323, 324, 326, 327, 329, 330, 332, 333, 335, 336, 338, 339, 341, 342, 343, 344, 345, 347, 348, 350, 351, 353, 354, 356, 357, 359, 360, 362, 363, 365, 366, 368, 370, 371, 373, 374, 376, 377, 379, 380, 382, 383, 385, 386, 388, 389, 391, 392, 394, 395, 397, 398, 400, 401, 403, 404, 406, 407, 409, and 410 or fragment thereof.

In certain embodiments, there is provided a polypeptide comprising the sequence of any one of SEQ ID NOs: 124, 125, 130, 133, 136, 139, 142, 145, 148, 151, 154, 157, 160, 163, 166, 169, 172, 176, 179, 183, 186, 189, 193, 197, 199, 202, 204, 207, 211, 214, 217, 220, 223, 226, 229, 232, 235, 238, 241, 244, 247, 250, 253, 256, 259, 262, 265, 268, 269, 270, 271, 272, 273, 274, 275, 276, 277, 278, 279, 280, 283, 286, 289, 292, 295, 298, 301, 304, 307, 310, 313, 316, 319, 322,325, 328, 331, 334, 337, 340, 343, 346, 349, 352, 355, 358, 361, 364, 367, 369, 372, 375, 378, 381, 384, 387, 390, 393, 396, 399, 402, 405, 408 or fragment thereof.

In some embodiments of the present invention, there is provided a polypeptide comprising the any one of the sequences set forth above comprising one or more substitutions, insertions and/or deletions. In specific embodiments, such proteins have the same biological activity as a polypeptide comprising reference sequence.

In other embodiments, there is provided a polypeptide comprising a sequence at least 50%, 55%, 60%, 65%, 70%, 75%, 80%, 85%, 90%, 95%, 96%, 97%, 98%, 99% (or more) percent identity to any one of the sequences set forth in SEQ ID NOs: 124, 125, 130, 133, 136, 139, 142, 145, 148, 151, 154, 157, 160, 163, 166, 169, 172, 176, 179, 183, 186, 189, 193, 197, 199, 202, 204, 207, 211, 214, 217, 220, 223, 226, 229, 232, 235, 238, 241, 244, 247, 250, 253, 256, 259, 262, 265, 268, 269, 270, 271, 272, 273, 274, 275, 276, 277, 278, 279, 280, 283, 286, 289, 292, 295, 298, 301, 304, 307, 310, 313, 316, 319, 322,325, 328, 331, 334, 337, 340, 343, 346, 349, 352, 355, 358, 361, 364, 367, 369, 372, 375, 378, 381, 384, 387, 390, 393, 396, 399, 402, 405, 408 and fragments thereof. In specific embodiments, such proteins have the same biological activity as the protein comprising reference sequence.

In certain embodiments, the present invention provides fusion proteins comprising the polypeptide of the present invention and a heterologous polypeptide. The heterologous sequences may include but are not limited to markers, including fluorescent markers such as GFP, herbicide and/or pest resistance proteins, such as Cry1Ac, Cry1Ca, Cry3Aa, EPSPS, GPR or GFR. In certain embodiments, the fusion polypeptide comprises a linker sequence between the polypeptides.

### Vectors

The present invention further provides vectors. In certain embodiments, there is provided expression vectors comprising the nucleic acids or expressing the polypeptides of the present invention. In certain embodiments, the expression vectors further comprise heterologous sequences. Such heterologous sequences may include but are not limited to sequences encoding fluorescent markers such as GFP, herbicide and/or pest resistance proteins. The heterologous sequences may be part of a fusion protein with the polypeptides of the present invention or expressed as a separate protein.

In certain embodiments, the present invention further provides vectors for CRISPR mediated DNA alteration. In such embodiments, one or more vectors express Cas9 and guide RNA. In certain embodiments where CRISPR is utilized to replace the promoter, the one or more vectors further provide the homology-directed repair (HDR) template containing the new promoter flanked by 100-500 bp of DNA sequences from the plant genome flanking the Cas9 cutting site on each side.

The recombinant expression vectors of the invention comprise a nucleic acid of the invention in a form suitable for expression in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel (1990). Regulatory sequences include those that direct constitutive expression of a nucleotide sequence in many types of host cell and those that direct expression of the nucleotide sequence only in certain host cells (e.g., tissue-specific regulatory sequences) or inducible promoters (e.g., induced in response to abiotic factors such as environmental conditions, heat, drought, nutrient status or physiological status of the cell or biotic such as pathogen responsive). Examples of suitable promoters include constitutive promoters and conditional promoters such as inducible promoters and tissue specific promoters. A worker skilled in the art would readily appreciate that conditional promoters such as drought inducible and tissue specific may be used to optimize the beneficial effect and to mitigate the undesirable side-effects.

In certain embodiments, the promoter comprises the sequence as set forth in SEQ ID NOs: 414, 415, 426, 427, 452, 453, 454, 455, 456, 457, 458, 459, 460 or 461. It will be appreciated by those skilled in the art that the design of the expression vector can depend on such factors as the choice of the host cell to be transformed, the level of expression of protein desired as well as timing and location of expression, etc. The expression vectors of the invention can be introduced into host cells to thereby produce proteins or peptides, including fusion proteins or peptides, encoded by nucleic acids as described herein.

The recombinant expression vectors of the invention can be designed for expression in prokaryotic or eukaryotic cells. Exemplary cells include but are not limited to bacterial cells such as Escherichia coli, insect cells (using baculovirus expression vectors), yeast cells, plant cells or mammalian cells. Suitable host cells are discussed further in Goeddel (1990). Alternatively, the recombinant expression vector can be transcribed and translated in vitro, for example using T7 promoter regulatory sequences and T7 polymerase.

In one embodiment, the nucleic acids of the present invention are expressed in plants cells using plant expression vectors. Examples of plant expression vectors systems include but are not limited to tumor inducing (Ti) plasmid or portion thereof found in Agrobacterium, cauliflower mosaic virus (CaMV) DNA and vectors such as pB1121.

For expression in plants, the recombinant expression cassette may contain in addition to the nucleic acid of interest, a promoter region that functions in a plant cell, a transcription initiation site (if the coding sequence to be transcribed lacks one), and optionally a transcription termination/polyadenylation sequence. The termination/polyadenylation region may be obtained from the same gene as the promoter sequence or may be obtained from different genes. Unique restriction enzyme sites at the 5′ and 3′ ends of the cassette are typically included to allow for easy insertion into a pre-existing vector.

Examples of suitable promoters include promoters from plant viruses such as the 35S promoter from cauliflower mosaic virus (CaMV) (Odell et al., 1985), promoters from genes such as rice actin (McElroy et al., 1990), ubiquitin (Christensen et al., 1992; pEMU (Last et al., 1991), MAS (Velten et al., 1984), maize H3 histone (Lepetit et al., 1992); and Atanassvoa et al., 1992), the 5′- or 3′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, ALS promoter, (WO 96/30530), a synthetic promoter, such as Rsyn7, SCP and UCP promoters, ribulose-1,3-diphosphate carboxylase, fruit-specific promoters, heat shock promoters, seed-specific promoters and other transcription initiation regions from various plant genes, for example, including the various opine initiation regions, such as for example, octopine, mannopine, and nopaline.

Additional regulatory elements that may be connected to a nucleic acid of the invention for expression in plant cells include terminators, polyadenylation sequences, and nucleic acid sequences encoding signal peptides that permit localization within a plant cell or secretion of the protein from the cell. Such regulatory elements and methods for adding or exchanging these elements with other regulatory elements are known and include, but are not limited to, 3′ termination and/or polyadenylation regions such as those of the Agrobacterium tumefaciens nopaline synthase (nos) gene (Bevan et al., 1983); the potato proteinase inhibitor II (PINII) gene (Keil et al., 1986) and hereby incorporated by reference); and An et al. (1989); and the CaMV 19S gene (Mogen et al., 1990).

Plant signal sequences, including, but not limited to, signal-peptide encoding DNA/RNA sequences which target proteins to the extracellular matrix of the plant cell (Dratewka-Kos et al., 1989) and the Nicotiana plumbaginifolia extension gene (De Loose et al., 1991), or signal peptides which target proteins to the vacuole like the sweet potato sporamin gene (Matsuoka et al., 1991) and the barley lectin gene (Wilkins et al., 1990), or signals which cause proteins to be secreted such as that of PRIb (Lund et al., 1992), or those which target proteins to the plastids such as that of rapeseed enoyl-ACP reductase (Verwoert et al., 1994) are useful in the invention.

In another embodiment, the recombinant expression vector is capable of directing expression of the nucleic acid preferentially in a particular cell type (e.g., tissue-specific regulatory elements are used to express the nucleic acid). Tissue-specific regulatory elements are known in the art.

For example, the promoter associated with a coding sequence identified in the TAIR data base as At2g44790 (P.sub.4790) is a root specific promoter.

Organ-specific promoters are also well known. For example, the chalcone synthase-A gene (van der Meer et al., 1990) or the dihydroflavonol-4-reductase (dfr) promoter (Elomaa et al., 1998) direct expression in specific floral tissues. Also available are the patatin class I promoter is transcriptionally activated only in the potato tuber and can be used to target gene expression in the tuber (Bevan, 1986). Another potato-specific promoter is the granule-bound starch synthase (GBSS) promoter (Visser et al., 1991).

Other organ-specific promoters appropriate for a desired target organ can be isolated using known procedures. These control sequences are generally associated with genes uniquely expressed in the desired organ. In a typical higher plant, each organ has thousands of mRNAs that are absent from other organ systems (reviewed in Goldberg, 1986).

In certain embodiments, the promoter is selected from the group consisting of pVaEF670, pVrEF027, pPsEF774 and pPsEF893.

In certain embodiments, the promoter comprises the sequence as set forth in any one of SEQ ID NOs: 414, 415, 426, 427, 452, 453, 454, 455, 456, 457, 458, 459, 460 and 461.

The resulting expression system or cassette is ligated into or otherwise constructed to be included in a recombinant vector which is appropriate for plant transformation. The vector may also contain a selectable marker gene by which transformed plant cells can be identified in culture. The marker gene may encode antibiotic resistance proteins. These markers include resistance to G418, hygromycin, bleomycin, kanamycin, and gentamicin. Alternatively, the marker gene may encode a herbicide tolerance protein that provides tolerance to glufosinate or glyphosate type herbicides. After transforming the plant cells, those cells having the vector will be identified by their ability to grow on a medium containing the particular antibiotic or herbicide. Replication sequences, of bacterial or viral origin, are generally also included to allow the vector to be cloned in a bacterial or phage host, preferably a broad host range prokaryotic origin of replication is included. A selectable marker for bacteria may also be included to allow selection of bacterial cells bearing the desired construct. Suitable prokaryotic selectable markers also include resistance to antibiotics such as kanamycin or tetracycline.

Other DNA sequences encoding additional functions may also be present in the vector, as is known in the art. For instance, in the case of Agrobacterium transformations, T-DNA sequences will also be included for subsequent transfer to plant chromosomes.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell.

### Cells and Plants:

Also provided are cells and plants having modified expression or activity of the polypeptides of the present invention. The cells may be in culture, in a plant tissue including for example, leaves, stems, shoots, roots, flowers, fruits and seeds or whole plant. Exemplary plant cells include but are not limited to epidermal cells, mesophyll and other ground tissues, and vascular tissues in leaves, stems, floral organs, and roots. Plants include plants at any stage of development. The cells and plants may be non-mutagenized, mutagenized or transgenic and the progeny thereof.

The plant can be any monocot or dicot. In certain embodiments, the plant is a commercial crop, produce crop, a biofuel crop, an ornamental plant, a flowering plant, an annual plant or a perennial plant. Examples of plants include but are not limited Arabidopsis thaliana, Brassica napus, Glycine max, Zea mays, Triticum aestivum, Brachypodium distachyon, Oryza sativa, Brassica oleracea, Brassica rapa, Gossypium hirsutum, Gossypium raimondii, Hordeum vulgare, Setaria italica, Sorghum bicolor, Miscanthus sinensis, Panicum virgatum, Solanum lycopersicum, Cucumis sativus, Lactuca sativa, Vigna nguiculata, Phaseolus vulgaris, Cicer arietinum, Daucus carota, Asparagus fficinalis, Solanum tuberosum, Carica papaya, Chenopodium quinoa, Malus omestica, Citrus sinensis, Vitis vinifera, Manihot esculenta, Theobroma cacao, Coffea arabica, Camellia sinesis, Olea europaea, Populus trichocarpa, Taraxacum kok-saghyz, Helianthus annuus and Petunia axillaris.

In certain embodiments, the cells and plants over-express the polypeptides of the present invention as compared to wild type cells and plants. In alternative embodiments, the cells and plants under express the polypeptides of the present invention. A worker skilled in the art would readily appreciate that endogenous expression of the polypeptides of the present invention can be modified.

In certain embodiments, endogenous expression or activity of the polypeptides of the present invention is modified by mutating the native promoter or gene encoding the polypeptide of the invention. A worker skilled in the art would readily appreciate methods for random or site directed mutations and methods of screening for mutants.

In certain embodiments, the plant has been modified using site-directed methodologies targeting the promoter and/or gene encoding the polypeptides of the invention. A worker skilled in the art would readily appreciate methods for mutating or genetically modifying the promoter or gene. For example, a worker skilled in the art would readily appreciate the CRISPR editing methodologies may be used. Non-limiting illustrative examples of CRISPR editing methodologies are detailed in the examples. A worker skilled in the art would readily appreciate that other examples of site-directed methods include but are not limited to methods utilizing meganucleases, TALENs and zinc finger nucleases In certain embodiments, the plants have been mutagenesized by chemical or physical means.

For example, a worker skilled in the art would readily appreciate that ethylmethane sulfonate (EMS) may be used as a mutagen or radiation, such as x-ray, y-ray, and fast-neutron radiation may be used as a mutagen.

In certain embodiments, endogenous expression of the polypeptides of the present invention is modified by replacing the native promoter with an alternative promoter, such as constitutive promoter, a dehydration-inducible promoter or tissue specific promoter. A worker skilled in the art would readily appreciate the CRISPR editing methodologies may be used to modify endogenous expression. Non-limiting illustrative examples of CRISPR editing methodologies are detailed in the examples.

In certain embodiments, there is provided cells and plants expressing exogenous polypeptides of the present invention. Non-limiting illustrative examples of methods utilizing vectors which express the polypeptides of the present invention are detailed in the examples.

In certain embodiments, the plants over expressing the polypeptides of the present invention have improved plant productivity, improved tolerance to water deficiency and/or nutrient deficiency as compared to wild type plants. In certain embodiments, the plants over expressing the polypeptides of the present invention have improved pollen viability under optimal and/or drought conditions as compare to wild type plants. In certain embodiments, the plants over expressing the polypeptides of the present invention have improved plant productivity under optimal and/or water and/or nutrient (including but not limited to nitrogen or phosphorous) deficient conditions as compared to wild type plants. Methods of measuring plant productivity are known in the art and include for example measuring shoot and/or root biomass.

### Methods:

The present invention further provides methods for producing the polypeptide of the present invention. In certain embodiments, the method comprises culturing a cell capable of expressing the nucleic acid of the invention in a suitable medium such that the polypeptide of the present invention is produced. A worker skilled in the art would readily appreciate that the cell may genetically modified to express or over express the nucleic acids of the present invention. In certain embodiments, the cells are genetically modified such that endogenous expression is modified by replacing the native promoter with an alternative promoter. In alternative embodiments, an expression vector expressing the polypeptide was introduced into the cell.

The present invention provides methods of improving plant productivity, tolerance to water deficiency and/or nutrient deficiency by over expressing the polypeptide of the present invention by upregulating endogenous expression and/or introducing an expression vector expressing the polypeptide in the plant. In certain embodiments, the expression vector expresses additional polypeptides or the additional expression vectors are introduced which express additional polypeptides, including for example proteins for herbicide resistance and/or pest resistance.

Also included in the invention are methods of producing a transgenic plant having of improved plant productivity, tolerance to water deficiency and/or nutrient deficiency by over expressing the polypeptide of the present invention by upregulating endogenous expression, for example by CRISPR mediated replacement of the native promoter and/or introducing an expression vector expressing the polypeptide in the plant. In certain embodiments, the plants are further modified to express additional polypeptides. For example, the expression vector may express additional polypeptides or expression vectors which express additional polypeptides are introduced, including for example proteins for herbicide resistance and/or pest resistance.

The plant can be any monocot or dicot. In certain embodiments, the plant is a commercial crop, produce crop, a biofuel crop, an ornamental plant, a flowering plant, an annual plant or a perennial plant. Examples of plants include but are not limited Arabidopsis thaliana, Brassica napus, Glycine max, Zea mays, Triticum aestivum, Brachypodium distachyon, Oryza sativa, Brassica oleracea, Brassica rapa, Gossypium hirsutum, Gossypium raimondii, Hordeum vulgare, Setaria italica, Sorghum bicolor, Miscanthus sinensis, Panicum virgatum, Solanum lycopersicum, Cucumis sativus, Lactuca sativa, Vigna nguiculata, Phaseolus vulgaris, Cicer arietinum, Daucus carota, Asparagus fficinalis, Solanum tuberosum, Carica papaya, Chenopodium quinoa, Malus omestica, Citrus sinensis, Vitis vinifera, Manihot esculenta, Theobroma cacao, Coffea arabica, Camellia sinesis, Olea europaea, Populus trichocarpa, Taraxacum kok-saghyz, Helianthus annuus and Petunia axillaris.

Numerous methods for introducing foreign nucleic acids into plants are known and can be used to insert a nucleic acid into a plant host, including biological and physical plant transformation protocols (See, for example, Miki et al., (1993) “Procedure for Introducing Foreign DNA into Plants”, In: Methods in Plant Molecular Biology and Biotechnology, Glick and Thompson, eds., CRC Press, Inc., Boca Raton, pages 67-88; and Andrew Bent in, Clough S J and Bent A F, (1998) “Floral dipping: a simplified method for Agrobacterium-mediated transformation of Arabidopsis thaliana”). The methods chosen vary with the host plant, and include chemical transfection methods such as calcium phosphate, polyethylene glycol (PEG) transformation, microorganism-mediated gene transfer such as Agrobacterium (Horsch et al., 1985), electroporation, protoplast transformation, micro-injection, flower dipping and biolistic bombardment.

Agrobacterium-Mediated Transformation The most widely utilized method for introducing an expression vector into plants is based on the natural transformation system of Agrobacterium tumefaciens and A. rhizogenes which are plant pathogenic bacteria which genetically transform plant cells. The Ti and Ri plasmids of A. tumefaciens and A. rhizogenes, respectfully, carry genes responsible for genetic transformation of plants (See, for example, Kado, 1991). Descriptions of the Agrobacterium vector systems and methods for Agrobacterium-mediated gene transfer are provided in Gruber et al. (1993). and Moloney et al., (1989).

Transgenic Arabidopsis plants can be produced easily by the method of dipping flowering plants into an Agrobacterium culture, based on the method of Andrew Bent in, Clough S J and Bent A F, 1998. Floral dipping: a simplified method for Agrobacterium-mediated transformation of Arabidopsis thaliana. Wild type plants are grown until the plant has both developing flowers and open flowers. The plants are inverted for 1 minute into a solution of Agrobacterium culture carrying the appropriate gene construct. Plants are then left horizontal in a tray and kept covered for two days to maintain humidity and then righted and bagged to continue growth and seed development. Mature seed is bulk harvested.

### Direct Gene Transfer

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes. (Sanford et al., 1993; Klein et al., 1992).

Plant transformation can also be achieved by the Aerosol Beam Injector (ABI) method described in U.S. Pat. Nos. 5,240,842 and 6,809,232. Aerosol beam technology is used to accelerate wet or dry particles to speeds enabling the particles to penetrate living cells. Aerosol beam technology employs the jet expansion of an inert gas as it passes from a region of higher gas pressure to a region of lower gas pressure through a small orifice. The expanding gas accelerates aerosol droplets, containing nucleic acid molecules to be introduced into a cell or tissue. The accelerated particles are positioned to impact a preferred target, for example a plant cell. The particles are constructed as droplets of a sufficiently small size so that the cell survives the penetration. The transformed cell or tissue is grown to produce a plant by standard techniques known to those in the applicable art.

### Regeneration of Transformants

The development or regeneration of plants from either single plant protoplasts or various explants is well known in the art (Weissbach and Weissbach, 1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a polypeptide of interest introduced by Agrobacterium from leaf explants can be achieved by methods well known in the art (see for example Horsch et al., 1985). In this procedure, transformants are cultured in the presence of a selection agent and in a medium that induces the regeneration of shoots in the plant strain being transformed as described (Fraley et al., 1983). In particular, U.S. Pat. No. 5,349,124 (specification incorporated herein by reference) details the creation of genetically transformed lettuce cells and plants resulting therefrom which express hybrid crystal proteins conferring insecticidal activity against Lepidopteran larvae to such plants.

This procedure typically produces shoots within two to four months and those shoots are then transferred to an appropriate root-inducing medium containing the selective agent and an antibiotic to prevent bacterial growth. Shoots that rooted in the presence of the selective agent to form plantlets are then transplanted to soil or other media to allow the production of roots. These procedures vary depending upon the particular plant strain employed, such variations being well known in the art.

Preferably, the regenerated plants are self-pollinated to provide homozygous transgenic plants, or pollen obtained from the regenerated plants is crossed to seed-grown plants of agronomically important, preferably inbred lines. Conversely, pollen from plants of those important lines is used to pollinate regenerated plants. A transgenic plant of the present invention containing a desired polypeptide is cultivated using methods well known to one skilled in the art.

A preferred transgenic plant is an independent segregate. A more preferred transgenic plant is homozygous. Seed from a transgenic plant may be grown in the field or greenhouse, and resulting sexually mature transgenic plants are self-pollinated to generate true breeding plants.

### EXAMPLES

### 1. Identification of Drought Tolerant Mutants Using a Unique Genetic Screening

In many plants, including Arabidopsis, occurrence of a drought stress during reproductive development results in dramatically reduced silique length, and as a consequence there is a reduction in overall seed production. As Arabidopsis continually produces siliques so a transient drought stress during flowering results in aborted or shortened siliques only in a limited section of the main inflorescence corresponding with the timing of the stress. Once plants are removed from the stress, newly produced siliques will develop normally so seeds can be obtained for further propagation and genetic analysis that may underline regulatory elements responsible for reproductive drought tolerance phenotypes. Using this assay, we screened a collection of about 27,000 Arabidopsis T-DNA activation tagged lines (Weigel, et al., 2000), and three mutants, d200-1, 6, and 7 from the same mutant pool, were identified in which the silique length was not dramatically affected by the stress, and it was able to maintain relatively constant. It is possible that the 3 mutants are simply siblings which will be determined by TAIL-PCR for T-DNA localisation.

These mutant candidates were then advanced to homozygosity that were used for further characterization such as detailed growth assessment under various conditions such as drought and nutrient deficits. Arabidopsis seeds from mutant and control plants were plated onto regular MS media, placed into the cold for 4 days and then into a grow chamber under optimal conditions (22° C.; 18 hr light, ˜200 μE). At one week out of the cold a total of 24 replicates per entry were transplanted into pre-weighed 3″ pot. There are a total of 8 replicates per entry per treatment (drought screen, optimal yield, drought yield). At 3 days into flowering, plants in the drought screen group were watered up to the same weight and weighed daily for 4 consecutive days before being harvested for leaf and stem biomass separately. Plants in the drought yield were also watered up to the same weight and maintained at this weight 2 periods of drought (5 days/each). The drought yield group all started around 3 days into flowering. Optimal plants were maintained under optimal conditions until mature, at which time both the optimal and drought yield groups were harvested for stem and seed biomass.

All statistical analysis was completed with a two-way ANOVA in JMP 7.0 and significant differences were identified using a student t-test at 10% level of significance.

The homozygous siblings of the d200 showed significant increase in shoot biomass accumulation at the end of 4-days of water withheld period comparing to their parental control CS907. More importantly, d200-1, 6 and 7 reduced water loss relative to their shoot dry weight by 19%, 13% and 16%, respectively at day 2, and 17%, 7% and 13%, respectively at day 3 comparing to their parental controls, indicating reduced transpiration of the mutants during the drought stress.

TABLE 1
Day 2 and Day 3 water lost relative to their biomass
accumulation for mutant d200s and controls.

| | | Days to | | | | | | | |
|---|---|---|---|---|---|---|---|---|---|
| | | 1st flower | | Shoot DW (g) - Day 0 | | | Shoot DW (g) - Day 4 | | |
| | | | Std | | Std | % | | Std | % |
| Entry | Rep | Mean | Err | Mean | Err | CS907 | Mean | Err | CS907 |
| d200-1 | 9 | 25.2 | 0.2 | 0.567 | 0.016 | 134% | 0.776 | 0.017 | 116% |
| d200-7 | 9 | 24.6 | 0.3 | 0.527 | 0.028 | 124% | 0.753 | 0.021 | 113% |
| d200-6 | 9 | 23.9 | 0.2 | 0.520 | 0.020 | 123% | 0.693 | 0.026 | 104% |
| CS907 | 9 | 217 | 0.4 | 0.424 | 0.025 | — | 0.668 | 0.018 | — |

| | | Water lost in 2 d/ | | | Water lost in 3 d/ | | |
|---|---|---|---|---|---|---|---|
| | | Shoot DW-d 4 | | | Shoot DW-d 4 | | |
| | | | Std | % | | Std | % |
| Entry | Rep | Mean | Err | CS907 | Mean | Err | CS907 |
| d200-1 | 9 | 67.5 | 2.3 | 81% | 93.8 | 2.5 | 83% |
| d200-7 | 9 | 70.3 | 0.7 | 84% | 98.0 | 1.9 | 87% |
| d200-6 | 8 | 72.9 | 1.5 | 87% | 104.3 | 2.2 | 93% |
| CS907 | 9 | 83.7 | 2.6 | 100% | 1124 | 21 | 100% |

(Bold - indicates significant differences to parent control CS907).

### 2. Mapping of Insertion Location in Arabidopsis Genome

Southern analysis of mutants d200-1, 6 and 7 showed identical banding pattern, suggesting that these mutants are siblings of the same d200 mutant. TAIL PCR was then performed to map the location of T-DNA insertion of the 35S-enhancer using genomic DNA of d200-1. Six arbitrary degenerate (AD) primers of 16 bases were designed to have 128- or 256-fold degenerate, which are able to anneal throughout genome (SEQ ID NO:1 to SEQ ID NO:6). The border primers pSK-35S-F4 (SEQ ID NO:10) and pSK-OCT-R4 (SEQ ID NO:14) specific to the left and right borders of T-DNA were paired with each of AD primers in primary PCR to amplify the genomic sequence near T-DNA insert. Following the primary arbitrary amplification, two nested PCRs were then performed on the diluted primary PCR product using the working AD primers and T-DNA-specific nest primers (e.g. pSK-35S-F3 (SEQ ID NO:9), pSK-35S-F2 (SEQ ID NO:8) and pSK-OCT-R3 (SEQ ID NO:13), pSK-OCT-R2 (SEQ ID NO:12) respectively). After three rounds of TAIL PCR using different nested primers, one specific DNA fragment was amplified from the d200 mutant.

The specific PCR product amplified from the genome of d200-1 was cleaned through affinity column and then subjected to Sanger sequencing using T-DNA-specific primers such as pSK-35S-F1 (SEQ ID NO:7), pSK-35S-F2 (SEQ ID NO:8), pSK-OCT-R1 (SEQ ID NO:11), pSK-OCT-R2 (SEQ ID NO:12). Sequencing results showed that the enhancer T-DNA is inserted in the intergenic region between At3g27970 and At3g27980 with the border sequence of SEQ ID NO:121. Thus, the T-DNA insert is 967 bp downstream the stop codon of At3g27970 and 1.4 kb upstream the start codon of At3g27980.

### 3. Activation of Genes Near the 35S-Enhancer-Tag

Total 8 genes are located within 10 kb range from the insertion of 35S-enhancer tag, including At3g27970 (zinc ion binding exonuclease family protein possibly involved in ribosomal RNA (rRNA) processing, named AtExo970 here), At3g27980 (plant pectin methylesterase inhibitor superfamily protein), At3g27990 (putative large non-coding RNA gene), At3g27950 (GDSL-motif esterase/acyltransferase), At3g27960 (Kinesin light chain-related 2, named AtKin960 here), At3g27968 (small unknown protein), At3g27997 (pseudogene) and At3g27999 (plant invertase/pectin methylesterase inhibitor superfamily protein). In order to quantify the effect of 35S enhancer-tag on the expression of these genes, total RNA was extracted from young rosette leaves of d200-1 and control wildtype Columbia (Col). RNase-free DNase was used to remove any genomic DNA contaminant from purified RNA, from which cDNA was synthesized using an oligo-dT primer. Gene-specific primers targeting the transcripts of all the 8 genes (SEQ ID NO:15 to SEQ ID NO:30) were used for RT-qPCR to quantify mRNA accumulation of these genes in young rosette leaves of the mutants in comparison of Col. Arabidopsis gene encoding protein phosphatase 2A subunit A3 (AtPP2AA3, SEQ ID NO:31, SEQ ID NO:32) was selected as internal reference due to its moderate and relatively stable expression in various tissues and under different conditions. RT-qPCR analysis showed that At3g27970 (named AtExo970 in this application) is significantly enhanced in d200-1 with 480 folds of increase in RNA accumulation when compared with the wildtype control. In order to precisely calculate the enhancement on AtExo970 mRNA accumulation, both forward and reverse primers (D200A970-qF3 (SEQ ID NO:23), D200A970-qR3 (SEQ ID NO:24)) were designed to span an intron based on AtExo970 genomic sequence. RT-qPCR using such primers showed that intron-free AtExo970 mRNA was barely detected in wildtype Col while being routinely amplified from d200. In d200 leaf cells, intron-free AtExo970 mRNA accumulated to an extreme level of 5971 folds of that in wildtype Col, whereas the expression of AtKin960 (SEQ ID NO:422; SEQ ID NO:423) was mildly elevated 4 times and the remaining 6 genes are not affected. Over-expression of AtExo970 in Arabidopsis recapitulated the phenotypes of d200s, whereas Over-expression of AtKin960 mildly improved drought tolerance of the transgenic Arabidopsis.

### 4. Functional Analysis of AtExo970 Gene

### 4.1 AtExo970 Gene Structure and Protein Properties

AtExo970 mRNA has a sequence of 1417 nucleotides (SEQ ID NO:122), which includes the coding region of 1074 nucleotides (SEQ ID NO:123), 5′ UTR region of 127 nucleotides (SEQ ID NO:126) and 3′ UTR region of 216 nucleotides (SEQ ID NO:127). AtExo970 gene encodes for a protein of 357 amino acids (SEQ ID NO:124). This protein contains 2 C2H2 Zinc finger motifs (“caacyrqfnklehlvehm” and “cgvckkhcrsfeslrehl”) at its N-terminus, which are involved in binding to RNA molecules; and a Rex4 catalytic domain of 165 residues at its C-terminus, which is responsible for an exoribonuclease activity. In addition, between the zinc finger motif and Rex4 catalytic domain, there is a predicted loose Heme-nitric oxide/oxygen binding (H-NOX) domain (SEQ ID NO:125) possibly functioning as sensor for gaseous signaling agent of nitric oxide (Domingos et al., 2015). The loose H-NOX motif in AtExo970 may play a role in signal transduction between environmental responses and ribosomal RNA processing or ribosome biogenesis.

AtExo970 has a very low basal expression in wildtype Arabidopsis. Its cDNA sequence was first reported to be isolated from hormone treated Arabidopsis callus (GenBank: Bx824546). In high throughput proteomic analysis, oligopeptides of AtExo970 protein were found in samples from cell culture, seeds and juvenile leaves.

A homolog of AtExo970 is located on chromosome 5 of Arabidopsis (At5g40310, named as AtExo310 in this application). AtExo310 shares 84% protein sequence identity with AtExo970 (SEQ ID NO:130) and 76% nucleotide sequence identity (SEQ ID NO:131).

### 4.2 Expression Profile of AtExo970 in Arabidopsis

In order to analyze the expression profile of AtExo970 in different organs or tissues, primers P7970-EcoRF (SEQ ID NO:33) And P7970-XhoR (SEQ ID NO:34) were designed to amplify the promoter sequence of AtExo970 (SEQ ID NO:128) using Col genomic DNA as template. The promoter of 708 bp was cloned upstream the GUS gene replacing 35S promoter in vector pEG-35S-IntrnGUS by EcoRI/XhoI double digestion, resulting in construct pEG-P7970-IntrnGUS.

Transgenic Arabidopsis plants carrying P7970-IntrnGUS cassette were obtained by flower-dipping transformation and routine line advancement. Young seedlings growing in MS medium or flowering plants growing in pots were used for GUS staining. As indicated by GUS staining, P7970 promoter from AtExo970 gene has strong activity in the roots of young seedlings and weak activity in true leaves. No GUS activity was seen in mature leaves and stems. In opening flowers, dark blue GUS staining was detected in the tip of flower stigma, where the fertilization happens.

### 4.3 Subcellular Localization of AtExo970 Protein in Arabidopsis

Phylogenetic-based Gene Ontology (GO) predicted that AtExo970 might be a nuclear-localized protein (Gaudet et al., 2011). In order to analyze the subcellular localization of AtExo970 in Arabidopsis, green fluorescence protein (eGFP) was fused to the N-terminal of AtExo970 protein. Two primers (AtExo970-XmaF, SEQ ID NO:35, and AtExo970-BamR, SEQ ID NO:36) were designed to amplify the coding sequence of AtExo970 (SEQ ID NO:123) from cloned AtExo970 cDNA. The insertion of AtExo970 CDS downstream eGFP sequence in pEGAD vector by BamHI-XmaI double digestion resulted in an in-frame fusion of eGFP with AtExo970 (pEGAD-35S-eGFP:AtExo970).

Homozygous T3 Arabidopsis plants carrying 35S-eGFP:AtExo970 cassette were obtained by flower-dipping transformation and subsequent generation advancement. Strong GFP signal was seen in roots or root hairs of young transgenic seedlings. Within root or root-hair cells, eGFP:AtExo970 was found in cytoplasm rather than nuclear. In the leaf epidermal cells, weak GFP signal was also seen in cytoplasm around large vacuole. Thus, AtExo970 is likely a cytoplasm-localized riboexonuclease involving in RNA processing or ribosome biogenesis in cytoplasm, however, further investigation is required to confirm the initial observation.

### 4.4 Response of AtExo970 to Environmental Factors

AtExo970 has a very low basal expression in Arabidopsis leaves and flowers that is barely detectable under normal growth condition. Analysis of AtExo970 promoter sequence (SEQ ID NO:128) showed the presence of a cis-element ((A)AACAAA(C)) at 2 different locations within the 708 bp-promoter. This cis-element possibly involves in gene expression in endosperm or under anaerobic growth condition. The closest homolog of AtExo970 in soybean (GmExo090, SEQ ID NO:173) also has a very low basal expression in soybean tissues. Analysis of GmExo090 promoter sequence (SEQ ID NO:413) showed that there are 7 repeats of the cis-element within the 2 kb-promoter sequence.

### 5. Loss-of-Function of AtExo970 in T-DNA Knockout Arabidopsis Line

A T-DNA insertion knockout line (GK-749C02) was ordered from ABRC (http://abrc.osu.edu/stocks/345062). GK-749C02 has T-DNA insertion at the 4^{th} exon of AtExo970 genomic DNA. A homozygous T4 sibling was confirmed by PCR using primers (D200A970-qF2, SEQ ID NO:20; AtExo970-SeqR1, SEQ ID NO:37) flunking the insert. T-DNA insertion breaks AtExo970 protein at 221^{st} residue, which falls into the middle of REX4 riboexonuclease domain.

Intact mRNA of AtExo970 was undetectable in GK-749C02 plants when primers flunking the insert (D200A970-qF2, SEQ ID NO:20; D200A970-qR4, SEQ ID NO:38) were used for qPCR. However, partial RNA fragment was detectable when primers upstream (D200A970-qF2, SEQ ID NO:20; D200A970-qR2, SEQ ID NO:22) or downstream (D200A970-qF3, SEQ ID NO:23; D200A970-qR3, SEQ ID NO:24) the insert was used.

Under optimal condition, GK-749C02 has no visible difference from its segregated nulls and wildtype Col in their development from seedlings to mature plants. This is in agreement with the observation that AtExo970 has an extreme low expression under normal growth conditions.

### 6. Overexpression of AtExo970 in Arabidopsis

### 6.1 Constitutive Overexpression of AtExo970 in Arabidopsis Improves Drought Tolerance and Productivity

A genomic DNA fragment of AtExo970 was amplified from wildtype Arabidopsis Col-0. Two primers (AtExo970-SalF2, SEQ ID NO:39; AtExo970-XbaR2, SEQ ID NO:40) were designed to amplify AtExo970 genomic sequence of 2065 bp (SEQ ID NO:129) from purified genomic DNA of Col-0. The amplified AtExo970 gDNA fragment was inserted into pEarleyGate binary vector downstream a 35S promoter by SaII-XbaI or XhoI-XbaI double digestion of PCR fragment and pEarleyGate plasmid respectively. This cloning resulted in the construct pEG-35S-gAtExo970.

Arabidopsis Col-0 plants were flower-dipped with Agrobacterium tumefaciens (EHA105) carrying pEG-35S-gAtExo970 plasmid. Homozygous T3 lines containing 35S-gAtExo970 cassette were obtained through routine line advancement. Single-insert transgenic lines were selected based on Southern blot and progeny segregation on MS medium containing Basta of 10 mg/L.

14 of T3 homozygous transgenic lines of 35S-gAtExo970 were used for further molecular and physiological analyses. qPCR using primer pair (D200A970-qF2, SEQ ID NO:20; D200A970-qR2, SEQ ID NO:22) showed that AtExo970 mRNA level in the transgenic lines increased to 456-625 folds of wildtype Col-0 in rosette leaves. Most of these lines also have AtExo970 expression 3-4 folds higher than the original mutant d200. We evaluated plant growth and development as well as their drought tolerance determined by plant water loss (mainly caused by transpiration) during drought stress and final seed yield post drought stress. Comparing to their parent control Col and segregated null, most transgenic lines, similar to mutant d200, delayed in flowering, increased in biomass accumulation, reduced in water loss relative to their biomass accumulation and had better protection of seed yield, confirming that AtExo970 was responsible for drought tolerance of d200, and overexpression of AtExo970 in Arabidopsis indeed improves drought tolerance. The representative results of 3 transgenic lines are presented in Table 2 and 3.

TABLE 2
Day 4 detailed biomass and water loss relative to their biomass parameters
for 3 lines of 35S-gAtExo970 and their controls (null and Col) along
with mutant d200-8 and its control (CS907)
Drought Screen - day 4

| | |
|---|---|
| Days to flower | Shoot DW (g) |

| Entry | Rep# | Mean | Std Err | Mean | Std Err | % null | % Col |
|---|---|---|---|---|---|---|---|
| 4-2 | 8 | 24.8 | 0.4 | 1.072 | 0.010 | 124% | 125% |
| 13-7 | 8 | 23.8 | 0.2 | 1.083 | 0.025 | 125% | 127% |
| 18-9 | 8 | 24.4 | 0.3 | 1.073 | 0.034 | 124% | 125% |
| Null | 8 | 20.9 | 0.3 | 0.863 | 0.026 | — | 97% |
| Col | 8 | 21.5 | 0.4 | 0.855 | 0.035 | 103% | — |
| 200-8 | 8 | 24.1 | 0.2 | 1.060 | 0.041 | — | 108% |
| CS907 | 8 | 22.4 | 0.3 | 0.978 | 0.027 | — | — |

| | | Water lost in 2 d/ | | | | Water lost in 3 d/ | | | | Water lost in 4 d/ | | | |
|---|---|---|---|---|---|---|---|---|---|---|---|---|---|
| | | Shoot DW-d 4 | | | | Shoot DW-d 4 | | | | Shoot DW-d 4 | | | |
| | | | Std | % | % | | Std | % | % | | Std | % | % |
| Entry | Rep# | Mean | Err | null | Col | Mean | Err | null | Col | Mean | Err | null | Col |
| 4-2 | 8 | 76.85 | 0.63 | 83% | 84% | 84.62 | 0.61 | 81% | 82% | 88.13 | 0.68 | 80% | 82% |
| 13-7 | 8 | 76.24 | 1.51 | 82% | 83% | 84.36 | 1.84 | 81% | 82% | 87.71 | 1.96 | 80% | 81% |
| 18-9 | 8 | 77.93 | 2.13 | 84% | 85% | 85.89 | 2.84 | 82% | 84% | 89.10 | 3.04 | 81% | 83% |
| Null | 8 | 92.72 | 3.53 | — | 101% | 104.79 | 3.09 | — | 102% | 110.05 | 3.23 | — | 102% |
| Col | 8 | 91.49 | 2.59 | 99% | — | 102.63 | 2.62 | 98% | — | 107.77 | 2.80 | 98% | — |
| 200-8 | 8 | 76.28 | 2.30 | — | 87% | 86.17 | 3.21 | — | 91% | 90.02 | 3.54 | — | 92% |
| CS907 | 8 | 87.23 | 2.40 | — | — | 94.97 | 2.66 | — | — | 98.09 | 2.86 | — | — |

(Bold - indicates significant difference to segregated null, italics - significant difference to Col).

TABLE 3
Yield parameters for representative lines of 35S-gAtExo970 and controls
(null and Col) along with mutant d200-8 and its controls (CS907)
under both optimal conditions and after drought stress.
Optimal

| | | Seed Yield (g) | | | Stem DW (g) | | |
|---|---|---|---|---|---|---|---|
| Entry | # Reps | Mean | Std Err | % col | Mean | Std Err | % col |
| 4-2 | 8 | 1.12 | 0.06 | 101% | 2.78 | 0.12 | 105% |
| 13-7 | 8 | 1.30 | 0.09 | 118% | 2.83 | 0.07 | 107% |
| 18-9 | 8 | 1.08 | 0.09 | 98% | 2.75 | 0.11 | 104% |
| Col | 8 | 1.10 | 0 16 | — | 2.64 | 0.10 | — |
| 200-8 | 8 | 1.35 | 0.06 | 92% | 2.90 | 0.14 | 99% |
| CS907 | 8 | 1.46 | 0.10 | — | 2.93 | 0.12 | — |

Drought

| | | Seed Yield (g) | | | | | Stem DW (g) | | | | |
|---|---|---|---|---|---|---|---|---|---|---|---|
| | | | Std | % | % | % | | Std | % | % | % |
| Entry | # Reps | Mean | Err | col | opt | prot'n | Mean | Err | col | opt | prot'n |
| 4-2 | 8 | 1.06 | 0.02 | 119% | 95% | 26% | 2.33 | 0.05 | 108% | 84% | 2% |
| 13-7 | 8 | 1.05 | 0.04 | 118% | 81% | 12% | 2.30 | 0.06 | 107% | 81% | −1% |
| 18-9 | 8 | 0.90 | 0.04 | 101% | 83% | 14% | 2.35 | 0.07 | 109% | 85% | 3% |
| Col | 8 | 0.89 | 0.03 | — | 69% | — | 2.16 | 0.09 | — | 82% | — |
| 200-8 | 8 | 1.18 | 0.07 | 116% | 87% | 17% | 2.50 | 0.09 | 118% | 86% | 14% |
| CS907 | 8 | 1.02 | 0.03 | — | 70% | — | 2.12 | 0.09 | — | 72% | — |

| | | Drought | | | | | | | |
|---|---|---|---|---|---|---|---|---|---|
| | | Total shoot DW | | | | | Total water lost/ | | |
| | | (seeds + stems) (g) | | | | | Shoot DW | | |
| | | | Std | % | % | % | | Std | % |
| Entry | # Reps | Mean | Err | col | opt | prot'n | Mean | Err | col |
| 4-2 | 8 | 3.39 | 0.16 | 111% | 87% | 9% | 85.63 | 1.65 | 85% |
| 13-7 | 8 | 3.35 | 0.19 | 110% | 81% | 3% | 87.15 | 2.64 | 87% |
| 18-9 | 8 | 3.25 | 0.21 | 107% | 85% | 7% | 88.66 | 2.56 | 88% |
| Col | 8 | 3.05 | 0.28: | — | 78% | — | 100.61 | 3.74 | — |
| 200-8 | 8 | 3.68 | 0.40 | 117% | 86% | 14% | 79.51 | 3.06 | 84% |
| CS907 | 8 | 3.14 | 0.28 | — | 72% | — | 95.14 | 3.51 | — |

(bold - indicates significant difference to segregated null control, italics - significant difference to Col, % prot'n = % protection as the difference from optimal conditions relative to the control).

### 6.2 Conditional Expression of AtExo970 in Arabidopsis

To mitigate the possible side-effect of constitutive over-expression of exonuclease on Arabidopsis growth and development such as delayed flowering under normal condition, AtRD29A promoter has been used to regulate the expression of AtExo970 in the construct of pEG-PRd29A-AtExo970 as described below, and root specific promoter will also be used to localize the effect.

AtExo970 cDNA fragment (SEQ ID NO:131) was PCR amplified from total RNA of d200-1 leaves using AtExo970-SalF1 (SEQ ID NO:47) and AtExo970-XbaR1 (SEQ ID NO:48) primers.

The PCR product was double digested with Sall and XbaI. For cloning convenience, pEG-PRd29A-BnExo317 from Section 8.2 was used as starting vector. BnExo317 sequence was first removed from pEG-PRd29A-BnExo317 by XhoI-XbaI double digestion, and then replaced with SaII-XbaI digested AtExo970 cDNA. The cloning yielded construct pEG-PRd29A-AtExo970.

Arabidopsis Col-0 plants were flower-dipped with A. tumefaciens (EHA105) carrying pEG-PRd29A-AtExo970 plasmid. Single-insert transgenic lines were selected based on Southern analysis and confirmed by progeny segregation of subsequent generations on MS medium containing Basta of 10 mg/L. T3 homozygous transgenic lines are being obtained through routine line advancement and will be used for molecular analysis and physiological assessment. Subsequently, plant growth & development as well as tolerance to drought will be evaluated.

### 6.3 Overexpression of AtExo970 in Transgenic Arabidopsis Increases ABA Sensitivity at Emergence of Cotyledon and First Leaf

For each ABA plate assessment, a total of 5 replicates of 30 seeds/plate per entry per ABA concentration were seeded. The plates were placed into the cold for 4 days and then into a grow chamber under optimal conditions (22° C.; 18 hr light, ˜200 μE). ABA concentrations of 0 μM ABA (optimal) and 1.0 μM ABA were used. Starting at 2 days out of the cold, germination was recorded on each plate for 5 consecutive days. Cotyledon emergence was recorded from the starting day for 5 consecutive days and leaf emergence were recorded as it started until 10 days, and the % of emergence (cotyledon and leaf) was then calculated. All statistical analysis was completed with a two-way ANOVA in JMP 7.0 and significant differences were identified using a student t-test at 10% level of significance.

Comparing to their controls, most transgenic and mutant d200-8 seeds germinated well close to 100% under optimal and in the presence of exogenous ABA, showing that overexpression of AtExo970 didn't affect ABA sensitivity at the germination level. However, most of the transgenic lines had slight delay in cotyledon and first leaf emergence in the presence of 0.5 uM and 1 uM ABA, indicating that overexpression of AtExo970 increased ABA sensitivity at these stages of plant growth and development. This result suggests that ABA sensitivity of these transgenic plants may play a role in their drought tolerance.

TABLE 4
% germination at day 5, % cotyledon emergence at day 5 and % leaf emergence
at day 10 for selected lines of 35S-gAtExo970 and mutant d200-8
Optimal

| | | % germination- d 5 | | % cotyledon - d 5 | | % leaf - d 10 | |
|---|---|---|---|---|---|---|---|
| # Reps | Entry | Mean | Std Err | Mean | Std Err | Mean | Std Err |
| 5 | 4-2 | 98.7% | 0.0 | 100.0% | 0.0 | 99.4% | 0.0 |
| 5 | 10-6 | 100.0% | 0.0 | 100.0% | 0.0 | 100.7% | 0.0 |
| 5 | 13-7 | 100.0% | 0.0 | 99.4% | 0.0 | 100.0% | 0.0 |
| 5 | 18-9 | 84.0% | 0.0 | 100.0% | 0.0 | 99.4% | 0.0 |
| 5 | Null | 100.0% | 0.0 | 100.0% | 0.0 | 99.4% | 0.0 |
| 5 | Col | 99.4% | 0.0 | 100.0% | 0.0 | 100.0% | 0.0 |
| 5 | 200-8 | 99.4% | 0.0 | 100.0% | 0.0 | 100.0% | 0.0 |
| 5 | Null | 100.0% | 0.0 | 99.4% | 0.0 | 100.0% | 0.0 |

1.0 μM ABA

| | | % germination- d 5 | | | | % cotyledon - d 5 | | | | % leaf - d 10 | | | |
|---|---|---|---|---|---|---|---|---|---|---|---|---|---|
| | | | Std | Diff to | Diff to | | Std | Diff to | Diff to | | Std | Diff to | Diff to |
| # Reps | Entry | Mean | Err | null | Col | Mean | Err | null | Col | Mean | Err | null | Col |
| 5 | 4-2 | 96.7% | 0.0 | −3% | −1% | 71.8% | 0.1 | 4% | −23% | 53.5% | 0.0 | −11% | −38% |
| 5 | 10-6 | 99.4% | 0.0 | −1% | 2% | 62.5% | 0.1 | −5% | −32% | 62.4% | 0.1 | −2% | −29% |
| 5 | 13-7 | 98.1% | 0.0 | −2% | 1% | 60.4% | 0.1 | −7% | −34% | 75.1% | 0.1 | 11% | −17% |
| 5 | 18-9 | 85.0% | 0.0 | −15% | −13% | 38.9% | 0.0 | −29% | −55% | 44.2% | 0.1 | −20% | −48% |
| 5 | Null | 100.0% | 0.0 | — | 3% | 67.7% | 0.0 | — | −27% | 64.6% | 0.1 | — | −27% |
| 5 | Col | 97.5% | 0.0 | −3% | — | 94.3% | 0.0 | 27% | — | 91.8% | 0.1 | 27% | — |
| 5 | 200-8 | 100.0% | 0.0 | 6% | 2% | 57.5% | 0.1 | −4% | −37% | 66.3% | 0.1 | 31% | −26% |
| 5 | Null | 94.2% | 0.0 | — | — | 61.1% | 0.1 | — | — | 35.4% | 0.1 | — | — |

(bold - indicates significant difference to segregated null, italics - significant difference to Col)

### 6.4 Overexpression of AtExo970 in Transgenic Arabidopsis Plants Improves Pollen Viability Under Optimal and Drought Conditions

Pollen samples were taken from the drought plants on day 2 (19 to 25% initial soil water content) and from the same developmental stage in the optimal group. Pollen sampled were counted as the number of germinated pollens out of 100 pollen grains. A total of three 100 counts for each of the 4 replicates sampled per entry were done. All of the data for this study was analyzed using a two-way ANOVA in JMP 7.0 and significant differences were identified using the Student-T test at 10% level of significance.

Under optimal condition, pollen germination rate of most transgenic lines was slightly higher comparing to their null control; under drought stress, the difference was significantly amplified, suggesting that overexpression of AtExo970 in transgenic Arabidopsis plants improves seed yield under drought stress at least partly by enhancing pollen viability of these plants. Pollen samples were taken from the drought plants on day 2 (19 to 25% initial soil water content) and from the same developmental stage in the optimal group. Pollen germination was evaluated. A total of three 100 counts for each of the 4 replicates sampled per entry were done. All of the data for this study was analyzed using a two-way ANOVA in JMP 7.0 and significant differences were identified using the Student-T test at 10% level of significance.

TABLE 5
Pollen viability under both optimal and drought conditions for the
best drought tolerant lines of pEG-35S-gAtExo970 and mutant d200-8

| | | Optimal | | | Drought - Day 2 | | |
|---|---|---|---|---|---|---|---|
| Entry | Rep# | Mean | Std Err | % null | Mean | Std Err | % null |
| 4-2 | 12 | 48.0 | 1.3 | 108% | 43.6 | 0.9 | 120% |
| 5-3 | 12 | 47.4 | 1.5 | 107% | 50.8 | 1.9 | 139% |
| 14-11 | 12 | 49.2 | 1.2 | 111% | 44.9 | 1.2 | 123% |
| 16-9 | 12 | 46.3 | 2.3 | 104% | 43.2 | 1.5 | 119% |
| 18-9 | 12 | 43.3 | 0.9 | 97% | 39.6 | 1.6 | 109% |
| Null | 12 | 40.45 | 3.15 | — | 36.3 | 1.5 | — |
| 200-8 | 12 | 53.7 | 1.2 | 119% | 39.8 | 2.0 | 108% |
| CS907 | 12 | 45.3 | 2.8 | 100% | 370 | 1:5 | 100% |

(bold - indicates significant differences to the segregated nulls).

### 6.5 Overexpression of AtExo970 in Transgenic Arabidopsis Plants Improves Plant Productivity Under Optimal and Deficient N or P

A hydroponic assessment of the selected drought tolerant lines (5-3, 4-2, 25-8, 18-9, 22-8, 14-11, 16-9) along with their control (null and Col) and the original mutant d200-8 along with its control (CS907) was conducted to evaluate plant growth under optimal, low nitrogen (1/10 optimal) and low phosphorus (1/100 optimal) conditions. Arabidopsis were directly seeded into 70% agar wells in the hydroponic trays. The trays placed in the cold (5° C.) for 3 days and then into a 3-tier grow chamber under optimal conditions (22° C.; 18 hr light, ˜200 μE). At 2 weeks out of the cold, water was replaced with a modified Hoagland's solution for each treatment: optimal, low P (1/100), low N (1/10). Nutrient solution was replaced twice/week and photographs taken at the start of the nutrient treatment, 1 week and 2 weeks into the stress. All plants were maintained in the nutrient treatments until 1 week into flowering, at which point each plant was harvested for both shoot and root biomass.

Under optimal conditions all the transgenic lines and d200-8 had significant higher shoot and root biomass comparing to their controls; under low nitrogen (1/10^{th} optimal) condition, the transgenic lines and d200-8 overall produced similar amount of shoot biomass, but significantly higher amount of root biomass comparing to their controls; under low phosphorus (1/100^{th} optimal) conditions, the amount of shoot biomass was unchanged overall, but again the yield of roots of the transgenic lines and d200-8 was significantly enhanced.

TABLE 6
Shoot biomass (g) and root biomass (g) of selected transgenic lines
and controls (null and Col) along with mutant d200-8 and its
controls (CS907) under optimal hydroponic conditions.
Optimal

| | | Shoot DW (g) | | | | Root DW (g) | | | |
|---|---|---|---|---|---|---|---|---|---|
| | | | Std | % | % | | Std | % | % |
| Entry | # reps | Mean | Err | Col | Null | Mean | Err | Col | Null |
| 14-11 | 8 | 0.259 | 0.020 | 170% | 176% | 0.033 | 0.002 | 220% | 206% |
| 16-9 | 9 | 0.217 | 0.017 | 143% | 148% | 0.027 | 0.002 | 177% | 169% |
| 18-9 | 9 | 0.220 | 0.011 | 144% | 149% | 0.025 | 0.003 | 168% | 156% |
| 22-8 | 9 | 0.258 | 0.015 | 169% | 175% | 0.031 | 0.002 | 205% | 194% |
| 25-8 | 9 | 0.214 | 0.018 | 141% | 146% | 0.027 | 0.003 | 180% | 169% |
| 4-2 | 9 | 0.256 | 0.017 | 168% | 174% | 0.034 | 0.003 | 225% | 213% |
| 5-3 | 9 | 0.206 | 0.016 | 135% | 140% | 0.022 | 0.002 | 145% | 138% |
| Null | 9 | 0.145 | 0.013 | 95% | — | 0.016 | 0.001 | 107% | — |
| Col | 8 | 0.153 | 0.019 | — | 104% | 0.015 | 0.002 | — | 94% |
| 200-8 | 9 | 0.210 | 0.016 | 164% | — | 0.030 | 0.003 | 214% | — |
| CS907 | 9 | 0.128 | 0.010 | — | — | 0.014 | 0.002 | — | — |

(bold - significant difference to own control, italics - significant difference to Col, * note: the mutant d200-8 is compared to the CS907 not Col).

TABLE 7
Shoot biomass (g) and root biomass (g) for selected transgenic lines
and controls along with the mutant d200-8 and its controls (null and
Col) under low nitrogen (1/10^{th} optimal) hydroponic conditions.
Low N (1/10th)

| | | shoot DW (g) | | | | Root DW (g) | | | |
|---|---|---|---|---|---|---|---|---|---|
| | | | Std | % | % | | Std | % | % |
| Entry | # reps | Mean | Err | Col | Null | Mean | Err | Col | Null |
| 14-11 | 8 | 0.060 | 0.004 | 99% | 94% | 0.025 | 0.003 | 191% | 192% |
| 16-9 | 9 | 0.060 | 0.005 | 98% | 94% | 0.024 | 0.003 | 181% | 185% |
| 18-9 | 9 | 0.072 | 0.004 | 118% | 113% | 0.022 | 0.002 | 171% | 169% |
| 22-8 | 9 | 0.058 | 0.003 | 95% | 91% | 0.021 | 0.002 | 159% | 162% |
| 25-8 | 9 | 0.068 | 0.004 | 112% | 106% | 0.019 | 0.002 | 148% | 146% |
| 4-2 | 9 | 0.071 | 0.004 | 115% | 111% | 0.028 | 0.002 | 214% | 215% |
| 5-3 | 9 | 0.052 | 0.004 | 85% | 81% | 0.017 | 0.002 | 132% | 131% |
| Null | 9 | 0.064 | 0.002 | 105% | — | 0.013 | 0.001 | 100% | — |
| Col | 9 | 0.061 | 0.002 | — | 95% | 0.013 | 0.001 | — | 100% |
| 200-8 | 9 | 0.063 | 0.003 | 103% | — | 0.021 | 0.001 | 183% | — |
| CS907 | 9 | 0.061 | 0.002 | — | — | 0.012 | 0.002 | 100% | — |

(bold - significant difference to null control, italics - significant difference to Col, * note: the at mutant is compared to the CS907 not Col)

TABLE 8
Shoot biomass (g) and root biomass (g) for selected transgenic lines
and controls along with the mutant d200-8 and its control under
low phosphorus (1/100^{th} optimal) hydroponic conditions.
Low P (1/100)

| | | shoot DW (g) | | | | Root DW (g) | | | |
|---|---|---|---|---|---|---|---|---|---|
| | | | Std | % | % | | Std | % | % |
| Entry | # reps | Mean | Err | Col | Null | Mean | Err | Col | Null |
| 14-11 | 8 | 0.068 | 0.006 | 84% | 87% | 0.020 | 0.002 | 126% | 131% |
| 16-9 | 9 | 0.077 | 0.009 | 95% | 98% | 0.022 | 0.002 | 137% | 142% |
| 18-9 | 9 | 0.084 | 0.006 | 103% | 107% | 0.022 | 0.002 | 137% | 142% |
| 22-8 | 9 | 0.084 | 0.005 | 103% | 107% | 0.023 | 0.002 | 141% | 147% |
| 25-8 | 9 | 0.075 | 0.008 | 92% | 96% | 0.016 | 0.004 | 99% | 103% |
| 4-2 | 9 | 0.088 | 0.003 | 108% | 112% | 0.025 | 0.002 | 152% | 158% |
| 5-3 | 9 | 0.063 | 0.004 | 77% | 80% | 0.017 | 0.002 | 106% | 111% |
| Null | 9 | 0.077 | 0.006 | 95% | — | 0.016 | 0.002 | 100% | — |
| Col | 9 | 0.081 | 0.003 | — | 105% | 0.016 | 0.001 | — | 100% |
| 200-8 | 9 | 0.078 | 0.005 | 113% | — | 0.022 | 0.001 | 168% | — |
| CS907 | 9 | 0.069 | 0.004 | — | — | 0.013 | 0.001 | 100% | — |

(bold - significant difference to null control, italics - significant difference to Col, * note: the at mutant is compared to the CS907 not Col).

### 7. Constitutive Over-Expression of AtExo310, a Sequence Homolog of AtExo970 in Arabidopsis for Improvement of Drought Tolerance

AExo310 (SEQ ID NO:130) is the close homolog of AtExo970 in Arabidopsis, which share an identity of 84% in their protein sequence. AtExo310 has an expression pattern similar to AtExo970 in various tissues. To further elucidate its function, the cDNA of AtExo310 (SEQ ID NO:131) was amplified using primers AtExo310-BglF2 (SEQ ID NO:41) and AtExo310-XbaR1 (SEQ ID NO:42) and then cloned into pEarelyGate binary vector by BgIII-XbaI and BamHI-XbaI digestion respectively. This cloning produced construct pEG-35S-AtExo310, in which AtExo310 was driven by 35S promoter.

Arabidopsis Col-0 plants were flower-dipped with A. tumefaciens (EHA105) carrying pEG-35S-AtExo310 plasmid. Homozygous T3 lines were obtained through routine line advancement. Single-insert transgenic lines were selected based on Southern blot and progeny segregation on MS medium containing Basta of 10 mg/L.

### 8. Over-Expression of BnExo317 in Arabidopsis Improves Drought Tolerance

### 8.1. Constitutive Over-Expression of BnExo317 in Arabidopsis

Blastp query of AtExo970 protein sequence on canola (Brassica napus) genome database (http://www.genoscope.cns.fr/blat-server/cgi-bin/colza/webBlat/) showed that there are 6 close homologs of AtExo970 in canola A or C genomes. They are BnaA06g31730D (named here as BnExo317, SEQ ID NO:133), BnaC02g37170D (named here as BnExo170, SEQ ID NO:136), BnaA02g29140D (named here as BnExo140, SEQ ID NO:139), BnaA09g02180D (named here as BnExo180, SEQ ID NO:142), BnaC07g24840D (named here as BnExo840, SEQ ID NO:145), and BnaC09g01580D (named here as BnExo580, SEQ ID NO:148), respectively. All the canola homologs consist of 357 amino acids, same as AtExo970 protein except for BnExo140, which has one amino acid less. AtExo970 shares a very high identity of 92-94% with these canola homologs.

The nucleotide sequences of cDNA and genomic DNA for BnExo317, BnExo170, BnExo140, BnExo180, BnExo840, and BnExo580 are listed as SEQ ID NO:134, SEQ ID NO:135, SEQ ID NO:137, SEQ ID NO:138, SEQ ID NO:140, SEQ ID NO:141, SEQ ID NO:143, SEQ ID NO:144, SEQ ID NO:146, SEQ ID NO:147, SEQ ID NO:149, SEQ ID NO:150.

Primer BnExo317-XhoF1 (SEQ ID NO:43) and BnExo317-XbaR1 (SEQ ID NO:44) were designed to target to the 5′ or 3′ UTR region of BnExo317 gene, which is the closest homolog to AtExo970. cDNA made from Canola flower total RNA was used to amplify the coding sequence of BnExo317 (BnExo317_cDNA, SEQ ID NO:134).

Amplified BnExo317_cDNA fragment was inserted into pEarleyGate binary vector downstream 35S promoter by XhoI-XbaI double digestion of PCR product and pEarleyGate plasmid. This cloning resulted in the construct pEG-35S-BnExo317.

Arabidopsis Col-0 plants were flower-dipped with A. tumefaciens (EHA105) carrying pEG-35S-BnExo317 plasmid, and the resultant single-insert transgenic lines selected based on Southern blot and progeny segregation on MS medium containing Basta of 10 mg/I, were advanced to homozygosity at T3. 14 T3 homozygous transgenic lines of 35S-BnExo317 were used for further molecular analysis and physiological assessment, and the results of representative lines are presented here.

On average, the transgenic lines flowered slightly later than the controls (Col and null). At day 0 prior to drought treatment, most of the transgenic lines had significantly greater shoot dry weight compared to the controls, after the 4-day drought, all lines had significantly increased shoot biomass compared to the controls (Table 9). All transgenic lines lost significantly less water relative to their dry weights comparing to the controls at days 3 and day 4 of drought, and most of the lines had better biomass accumulation and improved water use efficiency (WUE) compared to the controls (Table 10). Thus, the transgenic lines of 35S-BnExo317 closely mirror those of 35S-gAtExo970 with increased biomass and reduced water loss relative to their biomass during the course of drought stress, indicating BnExo317 is a functional homolog of AtExo970.

TABLE 9
Days to flowering and shoot biomass (DW) at day 0 and day 4 harvest
from lines of 35S-BnExo317 in comparison of control null and Col.
Biomass

| | | Days to | | | | | | | | | |
|---|---|---|---|---|---|---|---|---|---|---|---|
| | | 1st Flower | | Shoot DW (g) - day 0 | | | | Shoot DW (g) - day 4 drought | | | |
| | | | Std | | Std | % | % | | Std | % | % |
| Entry | # Reps | Mean | Err | Mean | Err | Col | Null | Mean | Err | Col | Null |
| 27-5 | 8 | 20.9 | 0.4 | 0.430 | 0.035 | 132% | 116% | 0.729 | 0.031 | 133% | 121% |
| 29-6 | 8 | 21.0 | 0.5 | 0.455 | 0.033 | 140% | 122% | 0.672 | 0.028 | 123% | 112% |
| 34-8 | 8 | 20.8 | 0.4 | 0.436 | 0.038 | 134% | 117% | 0.711 | 0.034 | 130% | 118% |
| 47-2 | 8 | 20.8 | 0.2 | 0.429 | 0.015 | 132% | 115% | 0.734 | 0.025 | 134% | 122% |
| 80-5 | 8 | 21.1 | 0.5 | 0.439 | 0.042 | 135% | 118% | 0.774 | 0.021 | 142% | 128% |
| 96-1 | 8 | 20.4 | 0.4 | 0.377 | 0.030 | 116% | 101% | 0.722 | 0.027 | 132% | 120% |
| Null | 8 | 20.3 | 03 | 0.409 | 0.031 | 126% | — | 0.621 | 0.031 | 114% | — |
| Col | 8 | 19.5 | 0.3 | 0.325 | 0.032 | — | 87% | 0.547 | 0.030 | — | 88% |

(Bold- significant difference to the null, italics - significant difference to Col)

TABLE 10
Water loss relative to biomass at day 3 and 4, biomass accumulation
and water use efficiency (WUE) for transgenic lines of
35S-BnExo317 in comparison of control null and Col
Drought

| | | Water lost in 3 d/ | | | | Water lost in 4 d/ | | | | Biomass Accum | | WUE (g shoot/ | |
|---|---|---|---|---|---|---|---|---|---|---|---|---|---|
| | | shoot DW-d 4 | | | | shoot DW-d 4 | | | | (d 0-d 4) | | kg H20) | |
| Entry | | | Std | % | % | | Std | % | % | | % | | % |
| Entry | # Reps | Mean | Err | Col | Null | Mean | Err | Col | Null | Mean | Null | Mean | Null |
| 27-5 | 8 | 92.5 | 2.4 | 75% | 85% | 110.2 | 2.5 | 74% | 82% | 0.30 | 130% | 3.57 | 124% |
| 29-6 | 8 | 94.7 | 2.8 | 77% | 87% | 117.5 | 3.4 | 79% | 87% | 0.22 | 94% | 2.77 | 96% |
| 34-8 | 8 | 91.2 | 2.6 | 74% | 83% | 115.8 | 5.9 | 78% | 86% | 0.27 | 119% | 3.39 | 117% |
| 47-2 | 8 | 90.4 | 2.1 | 73% | 83% | 110.0 | 2.5 | 74% | 82% | 0.31 | 132% | 3.80 | 132% |
| 80-5 | 8 | 92.0 | 1.6 | 75% | 84% | 108.2 | 2.1 | 73% | 81% | 0.34 | 145% | 4.01 | 139% |
| 96-1 | 8 | 95.0 | 3.5 | 77% | 87% | 116.8 | 5.4 | 79% | 87% | 0.34 | 149% | 4.20 | 145% |
| Null | 8 | 104.6 | 3.1 | 85% | — | 129.6 | 5.4 | 87% | — | 0.23 | — | 2.87 | — |
| Col | 8 | 123.1 | 4.6 | — | 113% | 148.5 | 6.6 | — | 115% | 0.22 | 96% | 2.78 | 96% |

(Bold- significant difference to own null, italics - significant difference to Col).

### 8.2. Conditional Expression of BnExo317 in Arabidopsis

To mitigate the possible side-effect of constitutive over-expression of exonuclease on Arabidopsis growth under normal condition, AtRD29A, a well characterized dehydration-inducible promoter (Kasuga et al., 2004), was selected to drive the expression of BnExo317 upon drought stress. To this end, 35S promoter in pEG-35S-BnExo317 was replaced with AtRd29A promoter.

Primer AtRd29A-Eco1F (SEQ ID NO:45) and AtRd29A-XhoF1 (SEQ ID NO:46) were designed to amplify AtRd29A promoter of 1172 bp (SEQ ID NO:210) from Arabidopsis genomic DNA. 35S enhancer and promoter was removed from pEG-35S-BnExo317 plasmid by EcoRI-XhoI double digestion, and then replaced with AtRd29A promoter DNA that has previously been double digested with EcoRI and XhoI. This cloning resulted in construct pEG-PRd29A-BnExo317.

Arabidopsis Col-0 plants were flower-dipped with A. tumefaciens (EHA105) carrying pEG-PRd29A-BnExo317 plasmid. Single-insert transgenic lines were selected based on Southern analysis and confirmed by progeny segregation of subsequent generations on MS medium containing Basta of 10 mg/L. T3 homozygous transgenic lines are being obtained through routine line advancement and will be used for molecular analysis and physiological assessment.

### 9. Over-Expression of GmExo090 in Arabidopsis

### 9.1 Over-Expression of GmExo090 in Arabidopsis Improves Drought Tolerance

Blast search of AtExo970 protein against soybean (Glycine max) Williams 82 Assembly 1 Annotation 1.1 Protein Sequences (www.soybase.org) retrieved 3 homologs in soybean: Glyma19g11090 (named here as GmExo090, SEQ ID NO:172), Glyma15g02690 (named here as GmExo690, SEQ ID NO:176) and Glyma13g42740 (named here as GmExo740, SEQ ID NO:179). AtExo970 shares an identity of 86%, 70% and 70% with GmExo090, GmExo690 and GmExo740, respectively. The closest homolog GmExo090 has 354 amino acids (aa) while GmExo690 and GmExo740 both have 366 aa. GmExo690 and GmExo740 are paralogue to each other with identity of 94%. They are probably derived from the same ancestor during soybean genome duplication. Thus, only GmExo090 and GmExo740 were representatively used for further studies.

The nucleotide sequence of cDNA and genomic DNA for GmExo090 (SEQ ID NO:173 SEQ ID NO:175) are 1384 and 2704 nt in length respectively. Primers were designed to target to the 5′ or 3′ UTR region of GmExo090 gene. Soybean cDNA was made by reverse transcription of total RNA from young leaves of cultivar Jack. Primers GmExo090-XhoF2 (SEQ ID NO:49) and GmExo090-XbaR2 (SEQ ID NO:50) were used to amplify the coding sequence of GmExo090 from leaf cDNA.

GmExo090 cDNA fragment (SEQ ID NO:174) was double digested with XhoI-XbaI and then ligated to pEarleyGate plasmid digested with the same enzymes. This cloning resulted in construct pEG-35S-GmExo090, in which GmExo090 is downstream 35S promoter.

Arabidopsis Col-0 plants were flower-dipped with A. tumefaciens (EHA105) carrying pEG-35S-GmExo090 plasmid. Single-insert transgenic lines were selected based on Southern analysis and confirmed by progeny segregation of subsequent generations on MS medium containing Basta of 10 mg/L. T3 homozygous transgenic lines were obtained through routine line advancement and were used for molecular analysis. Subsequently, plant growth & development as well as tolerance to drought of 14 lines were evaluated.

All transgenic lines had bigger biomass at day 0 prior to drought and at day 4 of drought (Table 11), more importantly, their water loss relative to their biomass was significantly reduced comparing to their controls. In addition, most of the transgenic lines also showed higher water use efficiency (WUE) (Table 12). The result suggests that GmExo090 is the functional homolog of AtExo970 in soybean.

TABLE 11
Days to flowering and shoot biomass (DW) at day 0 and day 4 harvest from
transgenic lines of 35S-GmExo090 in comparison of control null and Col
Biomass

| | | Days to | | | | | | | | | |
|---|---|---|---|---|---|---|---|---|---|---|---|
| | | 1st Flower | | Shoot DW (g) - day 0 | | | | Shoot DW (g) - day 4 drought | | | |
| | | | Std | | Std | % | % | | Std | % | % |
| Entry | # Reps | Mean | Err | Mean | Err | Null | Col | Mean | Err | Null | Col |
| 9-3 | 8 | 21.5 | 0.3 | 0.43 | 0.04 | 124% | 123% | 0.75 | 0.03 | 126% | 130% |
| 16-1 | 8 | 21.1 | 0.4 | 0.40 | 0.03 | 117% | 116% | 0.67 | 0.03 | 113% | 116% |
| 20-6 | 8 | 20.9 | 0.4 | 0.39 | 0.04 | 115% | 113% | 0.70 | 0.04 | 118% | 122% |
| 30-2 | 8 | 21.0 | 0.3 | 0.39 | 0.03 | 114% | 113% | 0.67 | 0.04 | 112% | 115% |
| 60-11 | 8 | 21.4 | 0.3 | 0.44 | 0.04 | 128% | 127% | 0.68 | 0.03 | 114% | 117% |
| Null | 8 | 20.3 | 0.3 | 0.34 | 0.03 | — | 97% | 0.59 | 0.03 | — | 102% |
| Col | 8 | 20.3 | 0.3 | 0.35 | 0.02 | 101% | — | 0.58 | 0.02 | 97% | — |

(Bold- significant difference to the null, italics - significant difference to Col).

TABLE 12
Water loss relative to biomass at day 3 and 4, biomass accumulation
and water use efficiency (WUE) for lines of 35S-GmExo090 in
comparison of control null and Col
Day 4 - Drought

| | | Water loss in 3 d/ | | | | Water loss in 4 d/ | | | | Biomass | | WUE (g shoot/ | |
|---|---|---|---|---|---|---|---|---|---|---|---|---|---|
| | | shoot DW-d 4 | | | | shoot DW-d 4 | | | | accumulation | | kg H20) | |
| | | | Std | % | % | | Std | % | % | | % | | % |
| Entry | # Reps | Mean | Err | Null | Col | Mean | Err | Null | Col | Mean | Null | Mean | Null |
| 9-3 | 8 | 110.9 | 2.9 | 77% | 75% | 119.6 | 3.7 | 78% | 76% | 0.32 | 145% | 3.6 | 130% |
| 16-1 | 8 | 125.3 | 4.5 | 87% | 85% | 135.0 | 5.7 | 88% | 86% | 0.27 | 121% | 3.0 | 108% |
| 20-6 | 8 | 116.0 | 6.8 | 81% | 78% | 130.9 | 8.8 | 85% | 83% | 0.31 | 139% | 3.5 | 124% |
| 30-2 | 8 | 129.0 | 6.6 | 90% | 87% | 137.6 | 7.2 | 89% | 88% | 0.28 | 124% | 3.1 | 110% |
| 60-11 | 8 | 124.1 | 5.9 | 86% | 84% | 134.1 | 6.7 | 87% | 85% | 0.24 | 107% | 2.7 | 96% |
| Null | 8 | 144.0 | 5.1 | — | 97% | 153 9 | 6.1 | — | 97% | 0.25 | — | 2.8 | — |
| Col | 8 | 148.1 | 4.5 | 103% | — | 157.2 | 5.2 | 102% | — | 0.23 | 104% | 2.6 | 92% |

(Bold- significant difference to the null, italics - significant difference to Col).

### 9.2 Over-Expression of GmExo090 in Arabidopsis Improves Plant Productivity Under Optimal, N & P Deficiency

Arabidopsis seeds from the best lines and controls of pEG-35S-GmExo090 (9-3, 16-1, 20-6, 27-1, 54-2, null and Columbia) and pEG-35S-GmExo740 (37-9, 53-6, 66-3, 74-8, null) were direct seeded into 80% agar wells in the hydroponic trays. The trays were covered with saran wrap and placed in the cold (5° C.) for 3 days and then into a 3-tier grow chamber under optimal conditions (22° C.; 18 hr light, ˜200 μE). The trays remained covered for 8 days and then covered with hard covers for an additional week to prevent drying out. During the 1^{st} 2 weeks of growth the trays were maintained in water (week 1) and then optimal Hoagland's (week 2). At 2 weeks out of the cold, the water was replaced with a modified Hoagland's solution for each treatment: optimal, low P (1/100), low N (1/10). Nutrient solution was replaced twice weekly and photographs were taken at the start of the nutrient treatment, 1 week and 2 weeks into the stress. All plants were maintained in the nutrient treatments until 1 week into flowering, at which point each plant was harvested for both shoot and root biomass. Non-destructive growth measurements (#stems, #branches, #pods, #leaves and rosette diameter) were also recorded and individual photographs taken. All statistical analysis was completed using a 2-way ANOVA and significant comparisons were made with Student-T test at 10% level of significance.

Under optimal condition, all lines had increased shoot and root biomass compared to the controls with statistical significance for more than half of lines. Under the low N (1/10^{th} optimal) condition, there were no significant differences for the shoot biomass, however, all the lines had significantly increased root biomass compared to both the null and Columbia controls by greater than 200%. Similarly, under the low P (1/100^{th}) conditions, all lines had significantly increased root biomass compared to both the null and Columbia, and some lines showed increased shoot biomass.

TABLE 13
Shoot biomass (g) and root biomass (g) for selected transgenic
lines and controls under optimal hydroponic conditions.

| | | Optimal | | | | | | | |
|---|---|---|---|---|---|---|---|---|---|
| | | shoot DW (g) | | | | Root DW (g) | | | |
| | | | Std | % | % | | Std | % | % |
| Entry | # reps | Mean | Err | null | Col | Mean | Err | null | Col |
| 9-3 | 9 | 0.17 | 0.01 | 111% | 107% | 0.024 | 0.002 | 101% | 90% |
| 16-1 | 9 | 0.18 | 0.01 | 120% | 114% | 0.026 | 0.002 | 111% | 99% |
| 20-6 | 9 | 0.17 | 0.01 | 115% | 110% | 0.029 | 0.003 | 124% | 111% |
| 27-1 | 9 | 0.20 | 0.01 | 131% | 125% | 0.029 | 0.002 | 121% | 108% |
| 54-2 | 9 | 0.18 | 0.02 | 122% | 116% | 0.030 | 0.002 | 126% | 113% |
| Null | 9 | 0.15 | 0.01 | — | 96% | 0.024 | 0.002 | — | 89% |
| Col | 9 | 0.16 | 0.01 | 105% | — | 0.027 | 0.002 | 112% | — |

(bold - significant difference to null control, italics - significant difference to Col.

TABLE 14
Shoot biomass (g) and root biomass (g) for selected
transgenic lines and controls under low nitrogen
((1/100^{th} optimal) hydroponic conditions.

| | | Low N (1/10th) | | | | | | | |
|---|---|---|---|---|---|---|---|---|---|
| | | shoot DW (g) | | | | Root DW (g) | | | |
| | | | Std | % | % | | Std | % | % |
| Entry | # reps | Mean | Err | null | Col | Mean | Err | null | Col |
| 9-3 | 8 | 0.062 | 0.003 | 98% | 100% | 0.023 | 0.003 | 231% | 225% |
| 16-1 | 9 | 0.062 | 0.004 | 98% | 100% | 0.025 | 0.002 | 250% | 243% |
| 20-6 | 9 | 0.065 | 0.003 | 103% | 105% | 0.027 | 0.003 | 278% | 271% |
| 27-1 | 9 | 0.065 | 0.003 | 103% | 105% | 0.023 | 0.002 | 238% | 231% |
| 54-2 | 9 | 0.063 | 0.002 | 100% | 102% | 0.029 | 0.003 | 295% | 287% |
| Null | 9 | 0.063 | 0.004 | — | 102% | 0.010 | 0.001 | — | 97% |
| col | 9 | 0.062 | 0:004 | 98% | — | 0.010 | 0.002 | 103% | — |

(bold - significant difference to null control, italics - significant difference to Col.

TABLE 15
Shoot biomass (g) and root biomass (g) for selected transgenic lines and
controls under low phosphorus ((1/100^{th} optimal) hydroponic conditions.

| | | Low P (1/100) | | | | | | | |
|---|---|---|---|---|---|---|---|---|---|
| | | shoot DW (g) | | | | Root DW (g) | | | |
| | | | Std | % | % | | Std | % | % |
| Entry | # reps | Mean | Err | null | Col | Mean | Err | null | Col |
| 9-3 | 9 | 0.050 | 0.004 | 122% | 113% | 0.022 | 0.002 | 244% | 204% |
| 16-1 | 9 | 0.054 | 0.003 | 130% | 121% | 0.024 | 0.003 | 263% | 219% |
| 20-6 | 9 | 0.044 | 0.005 | 106% | 98% | 0.016 | 0.002 | 175% | 146% |
| 27-1 | 8 | 0.055 | 0.005 | 135% | 125% | 0.019 | 0.002 | 203% | 170% |
| 54-2 | 9 | 0.048 | 0.003 | 118% | 109% | 0.019 | 0.001 | 205% | 172% |
| Null | 9 | 0.041 | 0.003 | — | 93% | 0.009 | 0.001 | — | 83% |
| Col | 8 | 0.044 | 0.005 | 108% | — | 0.011 | 0.002 | 120% | — |

(bold - significant difference to null control, italics - significant difference to Col.

### 10. Over-Expression of GmExo740 in Arabidopsis Improves Drought Tolerance and Productivity

The nucleotide sequence of cDNA and genomic DNA of GmExo740 (SEQ ID NO:180, SEQ ID NO:181) are 1693 and 3759 nt in length respectively. Primers were designed to target to the 5′ or 3′ UTR region of GmExo740 gene. Soybean cDNA was made by reverse transcription of total RNA from young leaves of cultivar Jack. Primers GmExo740-XhoF1 (SEQ ID NO:51) and GmExo740-XbaR1 (SEQ ID NO:52) were used to amplify the coding sequence of GmExo740 from leaf cDNA.

GmExo740 cDNA fragment was double digested with XhoI-XbaI and then ligated to pEarleyGate plasmid digested with the same enzymes. This cloning resulted in construct pEG-35S-GmExo740, in which GmExo740 cDNA (SEQ ID NO:182) is downstream 35S promoter.

Arabidopsis Col-0 plants were flower-dipped with A. tumefaciens (EHA105) carrying pEG-35S-GmExo740 plasmid. Single-insert transgenic lines were selected based on Southern analysis and confirmed by progeny segregation of subsequent generations on MS medium containing Basta of 10 mg/L. T3 homozygous transgenic lines were obtained through routine line advancement and were used for molecular analysis. Subsequently, plant growth & development as well as tolerance to drought of 14 lines were evaluated in comparison of the controls including Col parent and segregated nulls.

Most of the transgenic lines flowered slightly later than the parent and null controls, had increased shoot biomass at day 0 prior to drought, maintained the same trend after 4 days of drought (Table 16). More importantly, these transgenic lines showed reduced water loss relative to their biomass at day 3 and 4 of drought, higher biomass accumulation and better water use efficiency during the stress comparing to the controls (Table 17). These results suggest that GmExo740, similar to GmExo090 is another functional homolog of AtExo970 in soybean.

TABLE 16
Days to flowering and shoot biomass (DW) at day 0
and day 4 of drought for lines of 35S-GmExo740.
Biomass

| | | Days to | | | | | | | | | |
|---|---|---|---|---|---|---|---|---|---|---|---|
| | | 1st Flower | | Shoot DW (g)-Day 0 | | | | Shoot DW (g)-Day 4 drought | | | |
| | | | Std | | Std | % | % | | Std | % | % |
| Entry | # Reps | Mean | Err | Mean | Err | Null | Col | Mean | Err | Null | Col |
| 15-8 | 8 | 20.8 | 0.5 | 0.65 | 0.04 | 105% | 125% | 0.84 | 0.03 | 106% | 118% |
| 37-9 | 8 | 21.1 | 0.2 | 0.64 | 0.02 | 103% | 123% | 0.83 | 0.05 | 106% | 117% |
| 53-6 | 8 | 21.5 | 0.4 | 0.70 | 0.03 | 113% | 135% | 0.89 | 0.03 | 113% | 126% |
| 57-1 | 8 | 21.3 | 0.3 | 0.72 | 0.03 | 116% | 140% | 0.85 | 0.03 | 108% | 120% |
| 66-3 | 8 | 21.1 | 0.5 | 0.69 | 0.04 | 111% | 133% | 0.89 | 0.03 | 113% | 126% |
| 74-8 | 8 | 20.6 | 0.3 | 0.59 | 0.03 | 95% | 115% | 0.89 | 0.04 | 113% | 126% |
| Null | 8 | 20.8 | 0.3 | 0.62 | 0.03 | — | 120% | 0.79 | 0.05 | — | 111% |
| Col | 8 | 19.5 | 0.3 | 0:52 | 0.03 | 84% | — | 0.71 | 0 04 | 90% | — |

(Bold- significant difference to null, italics - significant difference to Col).

TABLE 17
Water lost relative to biomass at day 3 and 4, biomass accumulation
and water use efficiency (WUE) for lines of 35S-GmExo740.
Drought

| | | Water lost in 3 d/ | | | | Water lost in 4 d/ | | | | Biomass | | WUE (g shoot/ | |
|---|---|---|---|---|---|---|---|---|---|---|---|---|---|
| | | shoot DW-d 4 | | | | shoot DW-d 4 | | | | accumulation | | kg H20) | |
| | # | | Std | % | % | | Std | % | % | | % | | % |
| Entry | Reps | Mean | Err | Null | Col | Mean | Err | Null | Col | Mean | Null | Mean | Null |
| 15-8 | 8 | 105.2 | 3.7 | 96% | 85% | 110.0 | 4.0 | 96% | 84% | 0.19 | 110% | 9.17 | 104% |
| 37-9 | 7 | 101.2 | 2.8 | 93% | 82% | 105.9 | 2.9 | 92% | 81% | 0.19 | 110% | 9.06 | 103% |
| 53-6 | 8 | 98.6 | 2.5 | 90% | 80% | 103.2 | 2.8 | 90% | 79% | 0.19 | 111% | 9.75 | 110% |
| 57-1 | 8 | 104.1 | 3.8 | 95% | 84% | 108.6 | 4.0 | 95% | 83% | 0.13 | 74% | 9.30 | 105% |
| 66-3 | 8 | 99.3 | 3.3 | 91% | 81% | 103.5 | 3.6 | 90% | 79% | 0.20 | 117% | 9.74 | 110% |
| 74-8 | 8 | 99.3 | 4.2 | 91% | 81% | 104.0 | 4.3 | 91% | 80% | 0.29 | 170% | 9.73 | 110% |
| Null | 8 | 109.0 | 6.4 | — | 88% | 114.4 | 6.7 | — | 88% | 0.17 | — | 8.64 | — |
| col | 7 | 123.3 | 6.4 | 113% | — | 130.4 | 7.2 | 114% | — | 0.19 | 110% | 7.84 | 89% |

(Bold- significant difference to own null, italics - significant difference to Col).

### 11. Over-Expression of ZmExo846 in Arabidopsis

### 11.1. Over-Expression of ZmExo846 in Arabidopsis Improves Drought Tolerance

Blast search of AtExo970 protein against corn (Zea may) B73 FGS Translations 5b.60 for RefGen_v2 (www.maizegdb.org) found 2 homologs in B73: GRMZM2G127846 (named here as ZmExo846, SEQ ID NO:183) and GRMZM2G079807 (named here as ZmExo807, SEQ ID NO:186). ZmExo846 and ZmExo807 share an identity of 80% and 73% with AtExo970 respectively. ZmExo846, the closer homolog, has 336 aa while ZmExo807 has 350 aa. The nucleotide sequence of cDNA and genomic DNA for ZmExo846 and ZmExo807 are listed as SEQ ID NO:184, SEQ ID NO:185, SEQ ID NO:187, SEQ ID NO:188 respectively.

Corn seeds of variety F507 were germinated in liquid medium in Magenta box for 10 days. Leaf and root tissue were collected for RNA and genomic DNA preparation. Corn cDNA was made by reverse transcription of total RNA from young leaves. Primers were designed to target to 5′ or 3′ UTR region of ZmExo846 gene. Primer ZmExo846-XhoF1 (SEQ ID NO:53) and ZmExo846-XbaR1 (SEQ ID NO:55) were used to amplify ZmExo846 gene from leaf genomic DNA.

ZmExo846 gDNA fragment was double digested with XhoI-XbaI and then ligated to pEarleyGate plasmid digested with the same enzymes. This cloning resulted in construct pEG-35S-gZmExo846, in which ZmExo846 gDNA (SEQ ID NO:185) is downstream 35S promoter.

Arabidopsis Col-0 plants were flower-dipped with A. tumefaciens (EHA105) carrying pEG-35S-gZmExo846 plasmid. Single-insert transgenic lines were selected based on Southern analysis and confirmed by progeny segregation of subsequent generations on MS medium containing Basta of 10 mg/L. T3 homozygous transgenic lines were obtained through routine line advancement and were used for molecular analysis. Subsequently, plant growth & development as well as tolerance to drought of 14 lines were evaluated in comparison of the controls including Col parent and segregated nulls.

Most of the transgenic lines flowered slightly later than the parent and null controls, had increased shoot biomass at day 0 prior to drought, maintained the same trend after 4 days of drought (Table 18). More importantly, these transgenic lines showed reduced water loss relative to their biomass at day 3 and 4 of drought, higher biomass accumulation and better water use efficiency during the stress comparing to the controls (Table 19). These results suggest that ZmExo846 is a functional homolog of AtExo970 in corn.

TABLE 18
Days to flowering and shoot biomass (DW) at day 0
and day 4 of drought for lines of 35S-gZmExo846.
Biomass

| | | Days to | | | | | | | | | |
|---|---|---|---|---|---|---|---|---|---|---|---|
| | | 1st Flower | | Shoot DW (g) - Day 0 | | | | Shoot DW (g) - day 4 drought | | | |
| | | | Std | | Std | % | % | | Std | % | % |
| Entry | # Reps | Mean | Err | Mean | Err | Null | Col | Mean | Err | Null | Col |
| 10-10 | 8 | 18.0 | 0.4 | 0.649 | 0.035 | 112% | 113% | 1.003 | 0.034 | 122% | 115% |
| 17-9 | 8 | 19.3 | 0.3 | 0.782 | 0.017 | 134% | 136% | 1.078 | 0.036 | 131% | 123% |
| 18-3 | 8 | 17.5 | 0.3 | 0.623 | 0.029 | 107% | 108% | 0.990 | 0.048 | 120% | 113% |
| 20-1 | 8 | 17.6 | 0.3 | 0.620 | 0.023 | 107% | 108% | 0.991 | 0.033 | 120% | 113% |
| 78-3 | 8 | 18.8 | 0.3 | 0.725 | 0.033 | 125% | 126% | 1.027 | 0.025 | 125% | 118% |
| 82-7 | 8 | 17.0 | 0.3 | 0.586 | 0.024 | 101% | 102% | 0.958 | 0.053 | 116% | 110% |
| Null | 8 | 16.8 | 0.3 | 0.581 | 0.030 | — | 101% | 0.824 | 0.030 | — | 94% |
| Col | 8 | 16.8 | 0.3 | 0.574 | 0.020 | 99% | — | 0.873 | 0.035 | 104% | — |

(Bold- significant difference to the null, italics - significant difference to Col).

TABLE 19
Water loss relative to biomass at day 3 and 4 of drought, biomass accumulation
and water use efficiency (WUE) for lines of 35S-gZmExo846.
Water loss

| | | Water loss in 3 d/ | | | | Water loss in 4 d/ | | | | Biomass | | WUE (g shoot/ | |
|---|---|---|---|---|---|---|---|---|---|---|---|---|---|
| | | shoot DW-d 4 | | | | shoot DW-d 4 | | | | accumulation | | kg H20) | |
| | | | Std | % | % | | Std | % | % | | % | | % |
| Entry | # Reps | Mean | Err | Null | Col | Mean | Err | Null | Col | Mean | Null | Mean | Null |
| 10-10 | 8 | 80.6 | 1.8 | 83% | 86% | 86.8 | 2.3 | 82% | 87% | 0.35 | 146% | 4.09 | 145% |
| 17-9 | 8 | 76.9 | 2.2 | 79% | 82% | 82.0 | 2.5 | 78% | 82% | 0.30 | 125% | 3.38 | 120% |
| 18-3 | 8 | 83.8 | 3.5 | 86% | 90% | 89.8 | 4.1 | 85% | 89% | 0.37 | 154% | 4.19 | 149% |
| 20-1 | 8 | 81.7 | 1.7 | 84% | 87% | 88.2 | 2.6 | 83% | 88% | 0.37 | 154% | 4.27 | 151% |
| 78-3 | 8 | 80.0 | 1.5 | 82% | 86% | 85.4 | 1.9 | 81% | 85% | 0.30 | 125% | 3.46 | 123% |
| 82-7 | 8 | 85.2 | 3.5 | 87% | 91% | 92.5 | 4.7 | 88% | 92% | 0.37 | 154% | 4.28 | 152% |
| Null | 8 | 97.6 | 3.0 | 100% | 104% | 105.7 | 3.6 | — | 105% | 0.24 | — | 2.82 | — |
| Col | 8 | 93.4 | 3.2 | 97% | 100% | 100.4 | 3.8 | 96% | — | 0.30 | 115% | 3.45 | 115% |

(Bold- significant difference to own null, italics - significant difference to Col).

### 11.2. Over-Expression of ZmExo846 in Arabidopsis Improves Plant Productivity Under Optimal, and Low N & P Conditions

Arabidopsis seeds from the best lines and controls of pEG-35S-BnExo317 (24-12, 26-12, 48-7, 50-5, 80-5, null) and pEG-35S-gZmExo846 (6-7, 10-10, 17-9, 34-6, null, Columbia) were direct seeded into 0.8% agar wells in the hydroponic trays. The trays were covered with saran wrap and placed in the cold (5° C.) for 3 days and then into a 3-tier grow chamber under optimal conditions (22° C.; 18 hr light, ˜200 μE). The trays remained covered for 8 days and then covered with hard covers for an additional week to prevent drying out. The water was replaced twice during the first week and then replaced with optimal solution for all trays for one week. At 2 weeks out of the cold, the water was replaced with a modified Hoagland's solution for each treatment: optimal, low P (1/100), low N (1/10). Nutrient solution was replaced twice weekly and photographs were taken at the start of the nutrient treatment, 1 week and 2 weeks into the stress. All plants were maintained in the nutrient treatments until 1 week into flowering, at which point each plant was harvested for both shoot and root biomass. Non-destructive growth measurements (#stems, #branches, #pods, #leaves and rosette diameter) were also recorded and individual photographs taken. All statistical analysis was completed using a 2-way ANOVA and significant comparisons were made with Student-T test at 10% level of significance.

Under optimal conditions, all lines had significantly increased shoot and root biomass compared to both the null and Columbia (Table 20). In the low nitrogen (1/10th) treatment, all lines had increased shoot biomass compared to both the null and Columbia with significant difference for 1 line, however, all lines had significantly increased root biomass compared to both controls (Table 21). In the low phosphorus (1/100th) treatment, all the lines had increased shoot biomass compared to both the null and Columbia with significant difference for most of the lines, and all lines had significantly increased root biomass compared to both controls (Table 22).

TABLE 20
Shoot biomass (g) and root biomass (g) for selected transgenic
lines and controls under optimal hydroponic conditions.

| | | Optimal | | | | | | | |
|---|---|---|---|---|---|---|---|---|---|
| | | shoot DW (g) | | | | Root DW (g) | | | |
| Entry | # reps | Mean | Std Err | % null | % col | Mean | Std Err | % null | % col |
| 6-7 | 8 | 0.289 | 0.026 | 157% | 142% | 0.030 | 0.003 | 211% | 143% |
| 10-10 | 9 | 0.289 | 0.010 | 157% | 142% | 0.027 | 0.002 | 194% | 131% |
| 17-9 | 9 | 0.284 | 0.022 | 154% | 140% | 0.029 | 0.002 | 210% | 142% |
| 34-6 | 7 | 0.270 | 0.017 | 147% | 133% | 0.026 | 0.002 | 185% | 125% |
| Null | 9 | 0.184 | 0.022 | — | 90% | 0.014 | 0.002 | — | 68% |
| Col | 9 | 0.203 | 0.014 | 111% | — | 0.021 | 0.002 | 148% | — |

(bold - significant difference to null control, italics - significant difference to Col.

TABLE 21
Shoot biomass (g) and root biomass (g) for selected transgenic lines and
controls under low nitrogen ((1/100^{th} optimal) hydroponic conditions.

| | | Low N (1/10th) | | | | | | | |
|---|---|---|---|---|---|---|---|---|---|
| | | shoot DW (g) | | | | Root DW (g) | | | |
| Entry | # reps | Mean | Std Err | % null | % col | Mean | Std Err | % null | % col |
| 6-7 | 9 | 0.088 | 0.008 | 111% | 135% | 0.020 | 0.003 | 181% | 191% |
| 10-10 | 9 | 0.100 | 0.008 | 127% | 155% | 0.028 | 0.003 | 245% | 259% |
| 17-9 | 9 | 0.093 | 0.007 | 119% | 144% | 0.021 | 0.004 | 184% | 194% |
| 34-6 | 6 | 0.089 | 0.010 | 113% | 137% | 0.027 | 0.004 | 242% | 256% |
| Null | 8 | 0.079 | 0.005 | — | 121% | 0.011 | 0.001 | — | 106% |
| col | 9 | 0.065 | 0.004 | 82% | — | 0.011 | 0.002 | 95% | — |

(bold - significant difference to null control, italics - significant difference to Col.

TABLE 22
Shoot biomass (g) and root biomass (g) for selected transgenic lines and
controls under low phosphorus ((1/100th optimal) hydroponic conditions.

| | | Low P (1/100th) | | | | | | | |
|---|---|---|---|---|---|---|---|---|---|
| | | shoot DW (g) | | | | Root DW (g) | | | |
| Entry | # reps | Mean | Std Err | % null | % col | Mean | Std Err | % null | % col |
| 6-7 | 9 | 0.103 | 0.010 | 125% | 163% | 0.026 | 0.004 | 343% | 304% |
| 10-10 | 9 | 0.129 | 0.009 | 156% | 203% | 0.032 | 0.004 | 416% | 369% |
| 17-9 | 9 | 0.116 | 0.010 | 141% | 183% | 0.017 | 0.002 | 218% | 193% |
| 34-6 | 6 | 0.101 | 0.015 | 122% | 158% | 0.019 | 0.001 | 252% | 223% |
| Null | 9 | 0.083 | 0.007 | — | 130% | 0.008 | 0.001 | — | 89% |
| Col | 8 | 0.063 | 0.007 | 77% | — | 0.009 | 0.001 | 113% | — |

(bold - significant difference to null control, italics - significant difference to Col.

### 12. Over-Expression of TaExo220 in Arabidopsis

By Blasting AtExo970 protein against NCBI Wheat (Triticum aestivum) Non-redundant Protein Sequences (https://blast.ncbi.nlm.nih.gov/) and referring to Wheat JBrowse in International Wheat Genome Consortium (http://www.wheatgenome.org), several wheat homologs were identified from different wheat cultivars. Based on sequence similarity and integrity, 3 homologs from Chinese spring wheat were identified. They are TraesCS6B01G302200 (named here as TaExo220, SEQ ID NO:193), TraesCS4B01G319600 (named here as TaExo960, SEQ ID NO:199) and AK334506 (named here as TaExo506, SEQ ID NO:202).

TaExo220, TaExo960 and TaExo506 share an identity of 82%, 70% and 66% with AtExo970 respectively. TaExo220, the closest homolog, has 335 aa while TaExo960 and TaExo506 have 354 and 334 aa respectively. The nucleotide sequence of cDNA and genomic DNA for TaExo220 and TaExo960 are listed as SEQ ID NO:194, SEQ ID NO:195, SEQ ID NO:200 and SEQ ID NO:201 respectively. The cDNA sequence of TaExo506 is listed as SEQ ID NO:203.

Wheat seeds of variety Fielder 17 were germinated in liquid medium in Magenta box for 10 days. Leaf and stem tissue were collected for RNA and genomic DNA preparation. Wheat cDNA was made by reverse transcription of total RNA from young seedlings. Primers were designed to target to 5′ or 3′ UTR region of TaExo220 gene. Primer TaExo220-BglF1 (SEQ ID NO:60) and TaExo220-XbaR1 (SEQ ID NO:62) were used to amplify TaExo220 gene from leaf genomic DNA.

TaExo220 gDNA fragment was double digested with BgIII-XbaI and then ligated to pEarleyGate plasmid digested with BamHI and XbaI enzymes. This cloning resulted in construct pEG-35S-TaExo220, in which TaExo220 gDNA (SEQ ID NO:196) is downstream 35S promoter.

Sequencing of the cloned TaExo220 gDNA (SEQ ID NO:196) showed that TaExo220 from winter wheat Fielder 17 is slightly different from TaExo220 from Chinese spring wheat (95% identity, SEQ ID NO:195). TaExo220 cDNA sequence (SEQ ID NO:198) was then assembled from the genomic DNA sequence by removing 6 predicted introns. TaExo220-Fielder protein (SEQ ID NO:197) translated from the cDNA shares an identity of 99% with that from Chinese spring wheat. It was interesting to note that an alternative intron splicing may occur at first intron, which resulted in the 6 amino acid deletion.

Arabidopsis Col-0 plants were flower-dipped with A. tumefaciens (EHA105) carrying pEG-35S-TaExo220 plasmid. Single-insert transgenic lines were selected based on Southern analysis and confirmed by progeny segregation of subsequent generations on MS medium containing Basta of 10 mg/L. T3 homozygous transgenic lines were obtained through routine line advancement and were used for molecular analysis. Subsequently, plant growth, development and productivity as well as tolerance to drought and N & P deficiency of the transgenic lines are being evaluated in comparison of the controls including wild type and segregated nulls.

### 13. Over-Expression of BdExo960 in Arabidopsis

Blast search of AtExo970 protein against Brachypodium distachyon v3.1 proteome (https://phytozome.jgi.doe.gov) found 1 homolog in B. distachyon: Bradi3G52960 (named here as BdExo960, SEQ ID NO:189). BdExo960 shares an identity of 69% with AtExo970, which consists of 336 aa. The nucleotide sequence of cDNA and genomic DNA for BdExo960 is listed as SEQ ID NO:190, SEQ ID NO:191 respectively.

Primer BdExo960-XhoF1 (SEQ ID NO:56) and BdExo960-XbaR1 (SEQ ID NO:58) were designed to target to 5′ or 3′ UTR region of BdExo960 gene. B. distachyon cDNA was made by reverse transcription of total RNA from young leaves of Bd21. The 2 primers were used to amplify the coding sequence of BdExo960 from leaf cDNA.

BdExo960 cDNA fragment was double digested with XhoI-XbaI and then ligated to pEarleyGate plasmid digested with the same enzymes. This cloning resulted in construct pEG-35S-BdExo960, in which BdExo960 cDNA (SEQ ID NO:190) is downstream 35S promoter.

Arabidopsis Col-0 plants were flower-dipped with A. tumefaciens (EHA105) carrying pEG-35S-BdExo960 plasmid. Single-insert transgenic lines were selected based on Southern analysis and confirmed by progeny segregation of subsequent generations on MS medium containing Basta of 10 mg/L. T3 homozygous transgenic lines were obtained through routine line advancement and were used for molecular analysis. Subsequently, plant growth, development and productivity as well as tolerance to drought and N & P deficiency of the transgenic lines are being evaluated in comparison of the controls including wild type and segregated nulls.

14. Over-Expression of BdExo960 in Monocot Model Plant Brachypodium distachyon

Monocot model species Brachypodium distachyon (cultivar Bd21) was used for evaluation of over-expression of AtExo970 homologs from monocot species.

Brachypodium has 1 homolog of AtExo970 in its genome as described in Section 13. Two more primers (BdExo960-XbaF1, SEQ ID NO:57 and BdExo960-BglR1, SEQ ID NO:59) were designed to target to 5′ or 3′ UTR region of BdExo960 gene. The 2 primers were used to amplify BdExo960 coding sequence from leaf cDNA. cDNA fragment of BdExo960 was double digested with XbaI and BgIII enzymes and then ligated to pB1500-35S-GUS digested with XbaI and BamHI enzymes. This cloning resulted in construct pBl500-35S-BdExo960, in which BdExo960 cDNA (SEQ ID NO:192) is downstream 35S promoter. pB1500-35S-GUS binary vector contains BdGOS2 promoter-driven NPTII gene for Kanamycin or Paramomycin selection, which have been proven to be efficient for Brachypodium transformation.

Embryogenic calluse prepared from Brachypodium immature seeds (Bd21) were infected with A. tumefaciens (AGL1) carrying pBl500-35S-BdExo960 plasmid (Vogel et al., 2006). TO shoots were regenerated from paromycin-resistant callus on CIM medium. Homozygous single-copy T3 lines were selected based on Southern blot and progeny segregation on MS medium containing kanamycin of 50 mg/L. Subsequently, plant growth, development and productivity as well as tolerance to drought and N & P deficiency of the transgenic lines are being evaluated in comparison of the controls including wild type and segregated nulls.

### 15. Over-Expression of ZmExo846, OsExo920 or TaExo220 in Brachypodium

### 15.1 Over-Expression of ZmExo846 in Brachypodium

Maize has 2 homologs of AtExo970 as described in Section 11.1. They are ZmExo846 (SEQ ID NO:183) and ZmExo807 (SEQ ID NO:186). The closer homolog is ZmExo846 (SEQ ID NO:183) which share an identity of 80% with AtExo970. Two primers (ZmExo846-XbaF1, SEQ ID NO:482 and ZmExo846-BglR1, SEQ ID NO:483) were designed to target to 5′ or 3′ UTR region of ZmExo846 gene. The 2 primers were used to amplify ZmExo846 gene from leaf genomic DNA as described in Section 11.1.

Genomic DNA fragment of ZmExo846 (SEQ ID NO:185) was double digested with XbaI and BgIII enzymes and then ligated to pB1500-35S-GUS digested with XbaI and BamHI enzymes. This cloning resulted in construct pB1500-35S-gZmExo846, in which ZmExo846 gDNA (SEQ ID NO:185) is downstream 35S promoter.

Embryogenic calluse prepared from Brachypodium immature seeds (Bd21) were infected with A. tumefaciens (AGL1) carrying pB1500-35S-gZmExo846 plasmid (Vogel et al., 2006). TO shoots were regenerated from paromomycin-resistant callus on CIM medium. Homozygous single-copy T3 lines were selected based on Southern blot and progeny segregation on MS medium containing kanamycin of 50 mg/L. Subsequently, plant growth, development and productivity as well as tolerance to drought and N & P deficiency of the transgenic lines are being evaluated in comparison of the controls including wild type and segregated nulls.

### 15.2. Over-Expression of OsExo920 in Brachypodium

By Blasting AtExo970 protein against rice (Oryza sativa) v7_JGI proteome in Phytozome (https://phytozome.jgi.doe.gov/), 2 rice homologs were identified: Os02g47920 (Renamed as OsExo920, SEQ ID NO:204) and Os01g01770 (Renamed as OsExo770, SEQ ID NO:207), which were both annotated as C2H2 zinc finger protein. OsExo920 and OsExo770 have 336 and 334 amino acids respectively, which are 21 or 23 amino acids shorter than AtExo970. The sequences missing in rice homologs lie right in H-NOX domain, which is presumably involved in nitric oxide (NO) signalling.

OsExo920 and OsExo770 have a protein sequence of 80% and 69% identical to AtExo970 respectively. The nucleotide sequence of cDNA and genomic DNA for OsExo920 and OsExo770 are listed as SEQ ID NO:205, SEQ ID NO:206, SEQ ID NO:208 and SEQ ID NO:209 respectively.

Primer OsExo920-XbaF1 (SEQ ID NO:66) and OsExo920-XmaR1 (SEQ ID NO:67) were designed to target to 5′ or 3′ UTR region of OsExo920 gene. These 2 primers were used to amplify OsExo920 gene from rice genomic DNA.

Genomic DNA fragment of OsExo920 (SEQ ID NO:206) was double digested with XbaI and XmaI enzymes and then ligated to pB1500-35S-GUS, which has previously been linearized to have a filled-BamHI blunt end and a sticky XbaI end. This cloning resulted in construct pBl500-35S-OsExo920, in which OsExo920 gDNA (SEQ ID NO:206) is downstream 35S promoter.

Embryogenic calluse prepared from Brachypodium immature seeds (Bd21) were infected with A. tumefaciens (AGL1) carrying pBl500-35S-OsExo920 plasmid (Vogel et al., 2006). TO shoots were regenerated from paromomycin-resistant callus on CIM medium. Homozygous single-copy T3 lines were selected based on Southern blot and progeny segregation on MS medium containing kanamycin of 50 mg/L. Subsequently, plant growth, development and productivity as well as tolerance to drought and N & P deficiency of the transgenic lines are being evaluated in comparison of the controls including wild type and segregated nulls.

### 15.3. Over-Expression of TaExo220 in Brachypodium

Wheat has 3 homologs of AtExo970 as described in Section 12.0. They are TaExo220 (SEQ ID NO:193), TaExo960 (SEQ ID NO:199) and TaExo506 (SEQ ID NO:202). The closest homolog is TaExo220 (SEQ ID NO:193) which share an identity of 82% with AtExo970. Two primers (TaExo220-XbaF1, SEQ ID NO:61 and TaExo220-BglR1, SEQ ID NO:63) were designed to target to 5′ or 3′ UTR region of TaExo220 gene. The 2 primers were used to amplify TaExo220 gene from leaf genomic DNA.

Genomic DNA fragment of TaExo220 (SEQ ID NO:196) was double digested with XbaI and BgIII enzymes and then ligated to pB1500-35S-GUS digested with XbaI and BamHI enzymes. This cloning resulted in construct pB1500-35S-TaExo220, in which TaExo220 gDNA (SEQ ID NO:196) is downstream 35S promoter.

Embryogenic calluse prepared from Brachypodium immature seeds (Bd21) were infected with A. tumefaciens (AGL1) carrying pB1500-35S-TaExo220 plasmid (Vogel et al., 2006). TO shoots were regenerated from kanamycin-resistant callus on CIM medium. Homozygous single-copy T3 lines were selected based on Southern blot and progeny segregation on MS medium containing kanamycin of 50 mg/L. Subsequently, plant growth, development and productivity as well as tolerance to drought and N & P deficiency of the transgenic lines are being evaluated in comparison of the controls including wild type and segregated nulls.

### 16. Over-Expression of BdExo960, ZmExo846 or TaExo220 Improves Productivity and Drought Tolerance in Brachypodium

Drought-T1 Brachypodium transgenic seeds for selected lines from 35S-BdExo960, 35S-gZmExo846 and 35S-TaExo220 and the parent control were direct seeded into pre-weighed 3″ pots and placed into the cold for a 2-week vernalization period. The plants were then placed into a grow chamber under optimal conditions (22° C.; 18 h light; ˜300 μE). A total of 20 pots per entry were seeded with 2 seeds per pot. Early into growth, each plant was sampled for PCR and based on the PCR results positive and negative replicates for each entry were selected to create segregated nulls for each line. At 4 days into spiking each plant was watered up to the same weight, covered with aluminum foil, weighed daily for 6 consecutive days, and then harvested for shoot biomass. The data collected are calculated to determine overall biomass under drought stress and water loss relative to biomass for the transgenic lines compared to the controls. All statistical analysis was completed using a 2-way ANOVA and significant comparisons were made with Student-T test at 10% level of significance.

The transgenic lines showed a gentle trend of flowering later than the segregated null controls, however it is not statistically significant, and most lines also had increased shoot biomass at drought (Table 23 and 25). More importantly, they showed statistically significantly reduced water loss relative to their biomass at day 4, 5 and 6 of drought (Table 24 and 26). These results suggest that the homologs of AtExo970 from monocots, such as BdExo960 and ZmExo846, are functional orthologs of AtExo970, and the similar mechanism involving these genes operates in both dicot and monocot plant species, that can be regulated to enhance plant tolerance to drought by improving their expression level in all plant species.

TABLE 23
Days to flowering and shoot biomass (DW)
of drought for lines of 35S-BdExo960
35S-BdExo960

| | | Days to Flower | | Shoot DW (g) | | |
|---|---|---|---|---|---|---|
| Entry | Rep# | Mean | Std Err | Mean | Std Err | % Ctrl |
| 116b-1 | 10 | 25.1 | 0.4 | 0.56 | 0.03 | 140% |
| 116C-2a | 8 | 23.9 | 0.5 | 0.50 | 0.03 | 126% |
| 9-1a | 9 | 24.7 | 0.6 | 0.48 | 0.03 | 120% |
| 116B-2a | 10 | 22.7 | 0.4 | 0.43 | 0.03 | 109% |
| 116b-3 | 11 | 21.4 | 0.5 | 0.38 | 0.02 | 96% |
| Control | 36 | 23.4 | 0.7 | 0.40 | 0.03 | — |

(Bold- significant different comparing to own segregated null.

TABLE 24
Water loss relative to biomass at day 4, 5 and 6 of drought,
biomass accumulation for lines of 35S-BdExo960

| | | Water lost in 4 d/ | | | Water lost in 5 d/ | | | Water lost in 6 d/ | | |
|---|---|---|---|---|---|---|---|---|---|---|
| | | shoot DW - d 6 | | | shoot DW - d 6 | | | shoot DW -d 6 | | |
| | | | Std | % | | Std | % | | Std | % |
| Entry | Rep# | Mean | Err | Ctrl | Mean | Err | Ctrl | Mean | Err | Ctrl |
| 116b-1 | 10 | 77.32 | 2.52 | 82% | 83.64 | 3.57 | 79% | 88.02 | 4.42 | 76% |
| 116C-2a | 8 | 81.95 | 5.36 | 87% | 90.66 | 5.77 | 85% | 96.11 | 6.11 | 83% |
| 9-1a | 9 | 82.93 | 3.41 | 88% | 92.39 | 3.66 | 87% | 98.57 | 4.08 | 85% |
| 116B-2a | 10 | 81.89 | 3.49 | 87% | 93.89 | 3.64 | 88% | 101.96 | 4.20 | 88% |
| 116b-3 | 11 | 87.24 | 3.29 | 93% | 100.71 | 3.97 | 95% | 111.44 | 4.71 | 96% |
| Control | 36 | 93.78 | 5.44 | — | 106.15 | 6.62 | — | 115.51 | 8.03 | — |

(Bold- significant different comparing to own segregated null, italics - significant difference to Col).

TABLE 25
Days to flowering and shoot biomass (DW) of
drought for lines of 35S-gZmExo846.
35S-gZmExo846

| | | Days to flower | | Shoot DW (g) | | |
|---|---|---|---|---|---|---|
| Entry | Rep# | Mean | Std Err | Mean | Std Err | % Ctrl |
| 4-2b | 7 | 25.9 | 0.5 | 0.49 | 0.02 | 127% |
| 4-6a | 5 | 25.2 | 0.5 | 0.49 | 0.02 | 126% |
| 8-1a | 10 | 24.5 | 0.8 | 0.46 | 0.04 | 119% |
| 4-4a | 10 | 23.2 | 0.4 | 0.40 | 0.03 | 104% |
| Control | 16 | 23.3 | 0.3 | 0.41 | 0.03 | — |

(Bold- significant different comparing to own segregated null).

TABLE 26
Water loss relative to biomass at day 4, 5 and
6 of drought for lines of 35S-gZmExo846.

| | | Water lost in 4 d/ | | | Water lost in 5 d/ | | | Water lost in 6 d/ | | |
|---|---|---|---|---|---|---|---|---|---|---|
| | | shoot DW - d 6 | | | shoot DW -d 6 | | | shoot DW -d 6 | | |
| | | | Std | % | | Std | % | | Std | % |
| Entry | Rep# | Mean | Err | Ctrl | Mean | Err | Ctrl | Mean | Err | Ctrl |
| 4-2b | 7 | 79.29 | 3.89 | 84% | 88.66 | 3.77 | 82% | 94.70 | 3.82 | 81% |
| 4-6a | 5 | 81.27 | 4.34 | 86% | 90.16 | 4.10 | 84% | 95.71 | 4.23 | 82% |
| 8-1a | 10 | 87.08 | 3.51 | 93% | 95.73 | 4.60 | 89% | 102.19 | 5.79 | 87% |
| 4-4a | 10 | 79.45 | 3.80 | 84% | 92.11 | 4.70 | 86% | 101.18 | 5.64 | 86% |
| Control | 16 | 85.00 | 3.52 | — | 96.18 | 2.72 | — | 105.36 | 2.11 | — |

(Bold- significant difference to own segregated null).

Low Nutrient Root Assessment—T1 Brachypodium transgenic seeds for selected lines from 35S-BdExo960, 35S-gZmExo846 and 35S-TaExo220 and the parent control were plated onto large plates with MS media. The plates were placed into the cold for a 7-day vernalization period and then into a grow chamber under optimal conditions (22° C.; 18 h light; ˜300 μE). A total of 40 seeds for each entry were plated. As soon as possible each seedling was sampled for PCR and based on the PCR results both positive and negative seedlings were selected and transplanted into seed germination pouches with various nutrient solutions made with a modified Hoagland's recipe. A total of 8 positives per entry for each of the following treatments: optimal, low nitrogen, low phosphorus were transplanted. Two pooled nulls were also transplanted using PCR negative seedlings. Each seed pouch was saturated at the start with the appropriate nutrient solution and maintained with each solution for 3 weeks of growth, at which point each seedling was harvested for shoot and root biomass. This study is currently in progress. All statistical analysis was completed using a 2-way ANOVA and significant comparisons were made with Student-T test at 10% level of significance.

### 17. Over-Expression of GmExo090, GmExo740 or AtExo970 Using Constitutive Promoter as Well as Conditional Promoters in Soybean

The promising results of overexpressing AtExo970 and its orthologs including GmExo090 and GmExo740 in Arabidopsis suggest that the effect is universal among various plant species. Thus, Agrobacterium tumefaciens (EHA105) carrying pEG-35S-GmExo090 in Section 9, pEG-35S-GmExo740 Section 10 and pEG-35S-gAtExo970 in Section 6.1 were used for transformation in soybean. Overexpression of these genes under conditional promoters such as drought inducible and root specific were also evaluated in soybean. 17.1. Agrobacterium-mediated soybean transformation Soybean cv. Jack and A. tumefaciens strain EHA105 were used for soybean transformation. EHA105 cells were transformed to carry either of the binary plasmids of pEG-35S-GmExo090, pEG-35S-GmExo740 or pEG-35S-gAtExo970.

Cotyledon node (CN) explants prepared from germinating seeds were immerged in Agro infection medium (AIM) for 30 min, followed by cultivation on co-cultivation medium (CCM) for 3 days. After shoots induction on shoot induction medium (SIM) for 3 weeks, explants were transferred to shoot elongation medium (SEM) under Basta selection (6 mg/L) till to the formation of Basta-resistant young shoots.

T0 transformants were confirmed by Basta painting by applying 40 ul of 100 mg/L glufosinate onto newly opened young leaflets. The integrity of all transgenes was confirmed by PCRs specific to each element in T-DNA. Transgene copy number was determined by Southern-blotting by probing on GFR sequence. Homozygous single-copy transgenic lines are being evaluated in comparison of controls including wild type Jack and segregated nulls for plant growth, development and productivity as well as tolerance to drought and N & P deficiency.

### 17.2. Over-Expression of GmExo090 in Soybean Improves Plant Productivity Under Optimal, Drought and Low N & P Conditions

Drought-Soybean seeds of the homozygous lines of 35S-GmExo090 and parent controls were direct seeded into pre-weighed 4″ deep pots in a growth chamber under optimal conditions (22° C.; 14 hr light, ˜500 μE). The experiment used a complete random block design with 8 replicates per entry in each of 3 treatments: day 0 harvest, day 4 drought and day 6 drought, which started 5 days after first open flower. Plants in the day 0 group were harvested for shoot biomass and drought plants were watered up to saturation, covered with aluminum foil and weighed daily for 4 or 6 consecutive days before being harvested for shoot biomass. statistical analysis was completed using a 2-way ANOVA and significant comparisons were made with a Student's T at 10% level of significance.

Most of the transgenic lines showed increased shoot biomass under optimal condition and the shoot growth was improved further under drought stress comparing to the parent control (Table 27). More importantly, these transgenic lines had significantly reduced transpiratory water loss during the drought period as shown at day 4, 5 and 6 (Table 28).

TABLE 27
Detailed biomass for selected lines and their control start of
drought (day 0), after 4 days of drought and 6 days of drought.

| | | Days to flower | | Shoot DW (g) - day 0 | | | Shoot DW (g) - day 4 | | | Shoot DW (g) - day 6 | | |
|---|---|---|---|---|---|---|---|---|---|---|---|---|
| | | | Std | | Std | % | | Std | % | | Std | % |
| Entry | Rep# | Mean | Err | Mean | Err | Ctrl | Mean | Err | Ctrl | Mean | Err | Ctrl |
| 5 | 8 | 30 | 0.6 | 4.61 | 0.4 | 105% | 5.65 | 0.4 | 103% | 5.26 | 0.4 | 118% |
| 4 | 8 | 30.9 | 1.2 | 4.37 | 0.4 | 99% | 5.7 | 0.3 | 103% | 5.11 | 0.3 | 114% |
| 6 | 8 | 29.9 | 0.6 | 4.86 | 0.5 | 110% | 6.01 | 0.5 | 109% | 5.09 | 0.4 | 114% |
| 30 | 8 | 29.9 | 0.4 | 4.49 | 0.4 | 102% | 5.58 | 0.3 | 101% | 4.88 | 0.2 | 109% |
| 27 | 8 | 31.1 | 0.7 | 5.08 | 0.4 | 115% | 5.28 | 0.3 | 96% | 4.75 | 0.2 | 106% |
| 25 | 8 | 29.6 | 0.8 | 4.57 | 0.4 | 104% | 5.43 | 0.3 | 99% | 4.56 | 0.2 | 102% |
| Control | 8 | 30.1 | 0.6 | 4.41 | 0.4 | — | 5.5 | 0.3 | — | 4.47 | 0.3 | — |

(Bold - indicates significant difference to control).

TABLE 28
Water lost relative to biomass for selected lines and
their control over the course of 4-and 6-day drought.

| | | Water lost in 4 d/ | | | Water lost in 5 d/ | | | Water lost in 6 d/ | | |
|---|---|---|---|---|---|---|---|---|---|---|
| | | shoot DW - d 6 | | | shoot DW - d 6 | | | shoot DW - d 6 | | |
| | | | Std | % | | Std | % | | Std | % |
| Entry | Rep# | Mean | Err | Ctrl | Mean | Err | Ctrl | Mean | Err | Ctrl |
| 5 | 8 | 54.85 | 2.7 | 86% | 61.64 | 3.5 | 86% | 65.49 | 4 | 86% |
| 4 | 8 | 56.47 | 2.2 | 88% | 62.56 | 2.5 | 87% | 66.66 | 3 | 87% |
| 6 | 8 | 59.34 | 4.1 | 93% | 65.08 | 4.7 | 91% | 68.54 | 5.2 | 90% |
| 25 | 8 | 60.18 | 4.1 | 94% | 66.3 | 5 | 92% | 69.94 | 5.5 | 92% |
| 30 | 8 | 62.37 | 1.8 | 97% | 67.41 | 2.3 | 94% | 70.76 | 2.7 | 93% |
| 27 | 8 | 62.37 | 1.4 | 97% | 68.76 | 1.8 | 96% | 72.3 | 2.1 | 95% |
| Control | 8 | 64.08 | 3.1 | — | 71.74 | 3.5 | — | 76.25 | 3.8 | — |

(Bold - indicates significant difference to control).

Drought yield-Soybean seeds of homozygous transgenic lines of 35S-GmExo090 and the parent controls were direct seeded into pre-weighed 4″ shallow pots with 3 treatments: optimal yield, drought yield, and drought screen (10 reps/entry/treatment except 4-2, 4-3; 5 reps/entry/treatment) and placed directly into a grow chamber under optimal conditions (22° C.; 14 hrs light, ˜500 μE). At 4 days into flowering, plants in the drought treatments were watered up to and covered with foil. Drought screen plants were weighed daily for 5 consecutive days and then harvested for shoot biomass and root biomass. Drought yield plants were weighed daily until they reached <30-50% SWC (below 190 g) and then maintained at that level for 5 days before being re-watered and returned to optimal conditions. Optimal and drought yield plants were measured for non-destructive parameters 18 days from flowering. All statistical analysis was completed using a 2-way ANOVA and significant comparisons were made with a Student's T at 10% level of significance. Most of the lines had a trend of more pods under optimal condition and all lines had significant more pods at the point of assessment comparing to their parent control.

More pods are formed under both optimal and drought conditions with statistical significance under stress condition, indicating that overexpression of GmExo090 ultimately improves seed yield of the transgenic soybean plants (Table 29) and the same strategy can be applied to all plant species.

TABLE 29
Number of pods for selected lines and control under
optimal conditions and after drought stress.

| | | # visible pods | | | | | | |
|---|---|---|---|---|---|---|---|---|
| | | Optimal | | | Drought | | | |
| Entry | # Reps | Mean | Std Err | % Ctrl | Mean | Std Err | % Ctrl | % opt |
| 4 | 5 | 29.4 | 1.3 | 101% | 32.4 | 2.4 | 145% | 110% |
| 30 | 10 | 34.6 | 2.4 | 119% | 30.2 | 2.6 | 135% | 87% |
| 6 | 10 | 35.1 | 2.3 | 121% | 28.9 | 2.7 | 130% | 82% |
| 5 | 10 | 33.1 | 2.6 | 114% | 25.7 | 2.1 | 115% | 78% |
| Control | 10 | 29.1 | 2 | — | 22.3 | 2.3 | — | 77% |

(Bold - indicates significant difference to control).

Low N and P hydroponic-Soybean seeds of the homozygous transgenic lines of 35S-GmExo090 and parent control Jack were direct seeded into 36 cell soil trays and placed directly into a 3-teir grow chamber under optimal conditions (22° C.; 18 hr light, ˜200 μE). At early germination 18 replicates of each entry were transplanted into 250 ml glass bottles covered with aluminum foil and filled with modified Hoagland's solution to create optimal, low nitrogen (1/100^{th} optimal) and low phosphorus (1/10^{th} optimal) conditions. 6 replicates per entry were in each of three treatments. The nutrient solution was replaced twice weekly and topped up as necessary. All plants were maintained in the nutrient solutions for 3 weeks from transplants, at which point they were harvested for both shoot and root biomass. All statistical analysis was completed using a 2-way ANOVA and significant comparisons were made with a Student's T at 10% level of significance. Both lines had increased shoot and more significantly root biomass comparing to the parent control under optimal, low N and low P conditions.

Under optimal condition, the lines had slightly increased shoot and root biomass compared to the control; and under the low nitrogen and phosphorus treatment, these lines had increased shoot and root biomass compared to the control with statistically significant difference (Table 30).

TABLE 30
Shoot biomass, root length and root biomass for selected lines and control grown
hydroponically in optimal, low nitrogen and low phosphorus conditions.

| | | Shoot Biomass (g) | | | | | | | | | | | | |
|---|---|---|---|---|---|---|---|---|---|---|---|---|---|---|
| | | Optimal | | | Low N | | | | | Low P | | | | |
| | | | Std | % | | Std | % | % | % | | Std | % | % | % |
| Entry | Rep# | Mean | Err | Ctrl | Mean | Err | Ctrl | opt | prot'n | Mean | Err | Ctrl | opt | prot'n |
| 6 | 6 | 1.35 | 0.06 | 103% | 0.54 | 0.05 | 124% | 40% | 7% | 0.73 | 0.06 | 107% | 54% | 2% |
| 30 | 6 | 1.37 | 0.04 | 105% | 0.54 | 0.06 | 125% | 39% | 6% | 0.98 | 0.03 | 143% | 72% | 19% |
| Control | 6 | 1.3 | 0.06 | — | 0.44 | 0.02 | — | 33% | — | 0.69 | 0.05 | — | 53% | — |
| | | Root Biomass (g) | | | | | | | | | | | | |
| | | Optimal | | | Low N | | | | | Low P | | | | |
| | | | Std | % | | Std | % | % | % | | Std | % | % | % |
| Entry | Rep# | Mean | Err | Ctrl | Mean | Err | Ctrl | opt | prot'n | Mean | Err | Ctrl | opt | prot'n |
| 6 | 6 | 0.67 | 0.03 | 104% | 0.39 | 0.05 | 126% | 58% | 10% | 0.47 | 0.04 | 115% | 70% | 7% |
| 30 | 6 | 0.7 | 0.03 | 109% | 0.37 | 0.04 | 120% | 53% | 5% | 0.57 | 0.02 | 141% | 82% | 18% |
| Control | 6 | 0.64 | 0.03 | — | 0.31 | 0.01 | — | 48% | — | 0.41 | 0.03 | — | 63% | — |

(Bold - indicates significant difference to control; NSD = no significant differences)
(% prot'n = the difference from optimal conditions compared to the control).

### 18. Loss-of-Function of AtExo970 in Arabidopsis Via CRISPR Editing

To construct a plasmid vector carrying both sgRNA and Cas9 cassettes, the sequence of Cas9 was assembled downstream of an Arabidopsis ubiquitin promoter together with two customized sgRNA driven by Arabidopsis U6 promoter, respectively. Four guide RNA sequences were carefully selected to target the AtExo1 of AtExo970 within the coding region of exon 2, 4 and 5, respectively (gRNA1(Exon2), SEQ ID NO:68; gRNA2(Exon4): SEQ ID NO:69; gRNA3(Exon4): SEQ ID NO:70; gRNA4(Exon5): SEQ ID NO:71). For each sgRNA, a pair of complementary oligonucleotides was synthesized and annealed to generate double-stranded DNA oligonucleotide, which were subsequently integrated upstream of the sgRNA scaffolds in the plasmid vector. The fragment containing the sgRNA and Cas9 cassettes was subcloned into a binary vector which contains a hygromycin resistance gene as a selection marker. Agrobacterium GV3101 containing the constructs were used to transform Col and d200 mutant via floral dipping. T1 seedlings were selected on MS plates containing hygromycin. Genomic DNA from leaves of transgenic plants was used for PCR amplification of relevant regions with specific primers flanking the target sites. PCR amplicons were assessed by T7 Endonuclease I (T7EI) or Cell mismatch assays and Sanger sequencing to identify the plants with InDel mutation at the target loci. The sequencing chromatograms were carefully examined for exact patterns that might indicate monoallelic or diallelic mutations.

### 19. Up-Regulation of GmExo090 by CRISPR Mediated Promoter Knock-In in Soybean

To generate transgene-free soybean overexpressing AtExo970 homologs, a constitutive promoter or an inducible promoter will be introduced to the 5′ end of GmExo090. As this gene has demonstrated similar effects as AtEx970 when overexpressed in Arabidopsis. The genomic DNA sequence of GmExo090 and flanking sequence was downloaded from SoyBase. Up to 2kb sequence upstream of the TSS was analyzed by various promoter analysis tools to identify the motifs and TF binding sites. This helps to determine the region to place the new promoter.

Then, we use a comprehensive guide RNA selection and evaluation process to choose highly effective guide RNA while minimizing off-target effect. Lists of putative guide RNAs were first generated using web-based tools such as CRISPR-P (crispr.hzau.edu.cn/CRISPR2/), CRISPR-PLANT (www.genome.arizona.edu/crispr2/). These lists were crosschecked and went through local single-guide RNA(sgRNA) designing tool such as CRISPR-Local to narrow down to ˜10 candidates based on several criteria such as location, off-target potential etc. These candidates were further reviewed manually to check their homology, PAM efficiency etc to choose the final guide RNAs for each target.

The final constructs used for transformation are binary vectors contain four major components within the two T-DNA borders: 1, A plant codon optimized Cas9 driven by a constitutive promoter, an inducible promoter or a tissue specific promoters to express the Cas9 as needed in planta; 2, A cassette to express guide RNA which is driven by a type Ill RNA polymerase Ill promoter such as U6; 3, The HDR template containing the new promoter flanked by 100-500 bp of DNA sequences from soybean genome flanking the Cas9 cutting site on each side, respectively; 4, A selection marker for plant transformation such as BASTA and hygromycin. Agrobacterium harboring the final constructs will be used to transform soybean. PCR will be used to confirm the transgene in the TO plants. T1 plants will be screened by PCR using a set of primers extend beyond the junction of the introduced promoter and the original HDR template. Ideally, transgene-free plants containing the new promoter can be identified in the T1 or T2 population. If not, the transgenic plants containing the new promoter will be back crossed with Jack to remove the transgene. Once the desired genotype is identified, seeds will be advanced and further physiology study will be carried out to evaluate the effect of the GmExo090 under the control of the new promoters.

Constructs for CRISPR mediated promoter knock-in were generated and transformed into the soybean Jack cultivar: construct pEGC11-GmExo29HDR to insert the AtRd29A while construct pEGC11-GmExo990HDR to insert GmUBC990 promoter at the 5′ end of the endogenous GmExo090. Guide RNA GmExo090g1 (SEQ ID NO:72) is used to direct SpCas9 to generate a DSB at the −67 of ATG. Complementary oligos oGmExo090g1F (SEQ ID NO:73) & oGmExo090g1R (SEQ ID NO:74) were chemically synthesized. After annealing, they were cloned into Bbsl digested pU626A vector. The gRNA expression cassette was digested with restriction enzymes HindIII and XmaI and cloned into pGC11 vector, resulted in pGC11-GmExo. To generate the HDR template for the inducible AtRd29A promoter, primer set GmExo090 HDR-F (SEQ ID NO:75) and Exo090Rd29A5Lnk-R (SEQ ID NO:78) were used to amplify the 5′ HDR arm using Jack genomic DNA as a template. Primer set GmExo090HDR-R (SEQ ID NO:76) and Exo090Rd29A3Lnk-F (SEQ ID NO:79) were used to amplify the 3′ HDR arm using Jack genomic DNA as a template. Primer set Exo090Rd29A5Lnk-F (SEQ ID NO:77) and Exo090Rd29A3Lnk-R (SEQ ID NO:80) were used to amplify the AtRd29A promoter using Arabidopsis Col genomic DNA as a template. The three PCR fragments were assembled by two rounds of overlapping PCR and the final HDR templates is disclosed as GmExo29HDR (SEQ ID NO:411). GmExo29HDR was digested with KpnI and EcoRI and cloned into pGC11-GmExo, resulted in pGC11-GmExo29HDR. pGC11-GmExo29HDR was digested with HindIII and EcoRI and cloned into binary expression vector pEGHE, resulted in pEGC11-GmExo29HDR. To generate the HDR template for the constitutive GmUBC990 promoter, primer set GmExo090HDR-F (SEQ ID NO:75) and Exo090UBC9905Lnk-R (SEQ ID NO:82) were used to amplify the 5′ HDR arm using Jack genomic DNA as a template. Primer set GmExo090HDR-R (SEQ ID NO:76) and Exo090UBC9903Lnk-F (SEQ ID NO:83) were used to amplify the 3′ HDR arm using Jack genomic DNA as a template. Primer set Exo090UBC9905Lnk-F (SEQ ID NO:81) and Exo090UBC9903Lnk-R (SEQ ID NO:84) were used to amplify the GmUBC990 promoter using Jack genomic DNA as a template. The three PCR fragments were assembled by two rounds of overlapping PCR and the final HDR templates is disclosed as GmExo990HDR (SEQ ID NO:412). GmExo990HDR was digested with KpnI and EcoRI and cloned into pGC11-GmExo, resulted in pGC11-GmExo990HDR. pGC11-GmExo990HDR was digested with HindIII and EcoRI and cloned into binary expression vector pEGHE, resulted in pEGC11-GmExo990HDR.

The pEGC11-GmExo29HDR and pEGC11-GmExo990HDR constructs were transformed into soybean Jack via Agrobacterium mediated transformation, respectively. The transgenic lines were selected on Basta, recovered and advanced to T2. Transgene free T3 lines with homozygous AtRd29A or GmUBC990 promoter integration were selected. These lines were advanced to T4 and tested for plant growth, development and productivity as well as tolerance to drought and N & P deficiency of the transgenic lines are being evaluated in comparison of the controls including wild type and segregated nulls.

The pEGC11-GmExo29HDR and pEGC11-GmExo990HDR constructs were transformed into soybean Jack via Agrobacterium mediated transformation, respectively. The transgenic lines were selected on Basta, recovered and advanced to T2. Transgene free T3 lines with homozygous AtRd29A or GmUBC990 promoter integration were selected. These lines were advanced to T4 and tested for plant growth, development and productivity as well as tolerance to drought and N & P deficiency of the transgenic lines are being evaluated in comparison of the controls including wild type and segregated nulls.

20. Overexpression of AtExo970 and its Orthologs Such as GmExo090 can be Combined with herbicide resistance in crop plants for additive effects

To facilitate the application of this invention in crop plants for field farming, herbicide resistances to glyphosate (GPR) and glufosinate (GFR) were stacked with current invention. Binary constructs such as p6A0-BoG-35S-GmExo090, p6A0-BoG-pGmUBC990-GmExo090, p6A0-BoG-RD29A-GmExo090 and p7CA-BoG-pGmUBC990-GmExo090 were made to have the 2 herbicide cassettes as well as GmExo090 cassette in a single T-DNA for soybean transformation.

### 20.1. GPR Expression Cassette for Glyphosate Resistance

5-enolpyruvylshikimate-3-phosphate synthase (EPSPS) from Agrobacterium tumefaciens (CP4) has been used for glyphosate resistance in soybean. GPR-CDS (SEQ ID NO:421) was synthesized encoding a fusion protein named GPR (SEQ ID NO:418). GPR contains a CP4 fused with a chloroplast transit peptide AtBCP-CTP (SEQ ID NO:416, SEQ ID NO:417) of Arabidopsis biotin carboxyl-carrier protein (At5g16390). The GPR-CDS is 18% different from in patent of U.S. Pat. No. 5,633,435A.

5′ UTR sequence of 108 bp from AtBCP (SEQ ID NO:419) and 3′ UTR sequence of 168 bp from AtEPSPS (SEQ ID NO:420) were added respectively to 5′ and 3′ ends of GPR-CDS for optimal gene expression in planta.

Other chloroplast-targeting signal from Arabidopsis rubisco activase protein (AtRCA-CTP, At2G39730, SEQ ID NO:424, SEQ ID NO:425) was also used to replace AtBCP-CTP in GPR.

Soybean pGmEF240 promoter (SEQ ID NO:426) was characterized as strong constitutive promoter from Glyma19g07240 locus, which encodes an elongation factor. Primer pair GmEF240-SaI1-F (SEQ ID NO:85) and GmEF240-EcoR1-R (SEQ ID NO:86) was used to amplify pGmEF240 promoter DNA from soybean genomic DNA.

GPR cassette was first modified by adding XhoI and AvrII to its 5′ or 3′ end respectively using PCR primers of BcpGR-XhoI-F (SEQ ID NO:95) and BcpGR-AvrII-R (SEQ ID NO:96). pGmEF240 was then inserted upstream GPR cassette by XhoI-StuI double digestion. The 3′-UTR of tubulin gene of Glyma10g40150 (Tub-T, SEQ ID NO: 428) was amplified from soybean Jack gDNA using primers of tGmTub-AvrII-F (SEQ ID NO: 89) and tGmTub-Spe-R (SEQ ID NO: 90). Tub-T was cloned downstream GPR as terminator by AvrII-SpeI double digestion. These cloning resulted in the expression cassette of pGmEF240-GPR-Tub-T in p6A0-G construct.

Strong promoters from other legume species, such as pVaEF670 (SEQ ID NO:452), pVrEF027 (SEQ ID NO:453), pPsEF774 (SEQ ID NO:454) and pPsEF893 (SEQ ID NO:455), were also used to drive GPR expression in various constructs after confirmation of their promoter activity in driving GUS expression in Arabidopsis.

Promoter pVaEF670 (SEQ ID NO:452) contains 1763 nucleotides covering −1 to −1763 bps upstream start codon of Vigna angularis elongation factor 1-alpha gene (LOC108345670). pVaEF670 was amplified by PCR using primer pair pVaEF670-F1 and pVaEF670-R1. It was introduced into pEG-A35S-GUS vector to drive GUS gene. Transgenic Arabidopsis seedlings carrying pVaEF670-GUS cassette showed stronger GUS staining in airy parts than the seedlings carrying 35S-GUS. In roots, similar Gus expression was observed in seedlings carrying either pVaEF670-GUS or 35S-GUS.

Promoter pVrEF027 (SEQ ID NO:453) contains 1712 nucleotides, covering −1 to −1712 bps upstream start codon of Vigna radiata (var. radiate) elongation factor 1-alpha (LOC106771027) gene. pVrEF027 was amplified by PCR using primer pair pVrEF027-F1 and pVrEF0270-R1. It was introduced into pEG-A35S-GUS vector to drive GUS gene. Transgenic Arabidopsis seedlings carrying pVrEF027-GUS cassette showed stronger GUS staining than those carrying 35S-GUS in both roots and airy parts.

Promoter pPsEF774 (SEQ ID NO:454) contains 1562 nucleotides, covering from −1 to −1562 bps upstream start codon of an elongation factor derived from Pisum sativum (cultivar Gradus No 2) whole genome shotgun sequence PUCA012449774. pPsEF774 was amplified by PCR using primer pair pPsEF774-F1 and pPsEF774-R1. It was introduced into pEG-A35S-GUS vector to drive GUS gene. Transgenic Arabidopsis seedlings containing pPsEF774-GUS cassette have a similar GUS staining to those containing 35S-GUS in both roots and airy parts.

Promoter pPsEF893 (SEQ ID NO:455) contains 1783 nucleotides, covering from −1 to −1783 bps upstream start codon of an elongation factor derived from Pisum sativum (cultivar Gradus No 2) whole genome shotgun sequence PUCA013332893. pPsEF893 was amplified by PCR using primer pair pPsEF893-F1 and pPsEF893-R1. It was introduced into pEG-A35S-GUS vector to drive GUS gene. Transgenic Arabidopsis seedlings carrying pPsEF893-GUS cassette have weaker GUS staining than those carrying 35S-GUS in both roots and airy parts.

### 20.2. GFR Expression Cassette for Glufosinate Resistance

Phosphinothricin acetyl transferase from Streptomyces hygroscopicus renders resistance to glufosinate (named GFR here). An optimized GFR-CDS (SEQ ID NO:429) was synthesized encoding GFR (SEQ ID NO:430). GFR-CDS is 29% different from to that in U.S. Pat. No. 5,561,236A. Four nucleotides (AACA) were added upstream ATG to facilitate translation initiation in plants.

GFR was first modified by adding Pacl, Sall and EcoRI to its 5′ end for cloning convenience. GFR was amplified using primers of BarOp-EcoR1-F (SEQ ID NO:91) and BarOp-BstBI-R (SEQ ID NO:92), and then incorporated into p6A0-G via EcoRI-BstBI double digestion. 35S promoter was put upstream GFR via SaII-EcoRI double digestion. 3′-UTR of soybean ribosome gene Glyma08g17870 (Rib-T, SEQ ID NO:431) was amplified from soybean Jack genomic DNA using primers of GmRibT-BstBI-F (SEQ ID NO:93) and GmRibT-StuI-R (SEQ ID NO:94), and then cloned into BstBI site downstream GFR as terminator. These cloning resulted in the expression cassette of 35S-GFR-RibT in p6A0-BoG construct.

Stronger promoter pPvEF2 (Phvul.004G075100, SEQ ID NO:427) from common bean was also used to drive GPR and pGmEF240 to drive GFR, which resulted in construct p7CA-BoG. Primer pair of PvEF2-StuI-F (SEQ ID NO:87) and PvEF2-XhoI-R (SEQ ID NO:88) was used to amplify pPvEF2 promoter from common bean genomic DNA. pGmEF240 (SEQ ID NO:426) in p6A0-BoG was then replaced by pPvEF2 (SEQ ID NO:427) via StuI-XhoI double digestion, and 35S by pGmEF240 via SaI1-EcoRI digestion. Promoter of soybean gene Glyma03g30110 (GmAct110, SEQ ID NO:414) and Glyma09g40960 (GmUBC960, SEQ ID NO:415) were also tested for GFR expression.

Promoters from other legume species, such as pPsEF817 (SEQ ID NO:456) and pVaEF895 (SEQ ID NO:457), were also analyzed for driving GFR expression in various constructs after confirmation of their promoter activity in driving GUS expression in Arabidopsis.

Promoter pPsEF817 (SEQ ID NO:456) contains 1801 nucleotides, covering from −1 to −1802 bps upstream start codon of an elongation factor derived from Pisum sativum (cultivar Gradus No 2) whole genome shotgun sequence PUCA012012817. pPsEF817 was amplified by PCR using primer pair pPsEF817-F1 and pPsEF817-R1. It was introduced into pEG-A35S-GUS vector to drive GUS gene. Transgenic Arabidopsis seedlings carrying pPsEF817-GUS cassette have a GUS expression stronger than those carrying 35S-GUS in both roots and airy parts.

Promoter pVaEF895 (SEQ ID NO:457) contains 1749 nucleotides, covering −1 to −1749 bps upstream start codon of Vigna angularis elongation factor 1-alpha-like (LOC108325895) gene. pVaEF895 was amplified by PCR using primer pair pVaEF895-F1 and pVaEF895-R1. It was introduced into pEG-A35S-GUS vector to drive GUS gene. Transgenic Arabidopsis seedlings carrying pVaEF895-GUS cassette showed faint Gus staining in both root and airy parts, much weaker than control seedlings carrying 35S-GUS.

Promoter pPcEF357 (SEQ ID NO:458) contains 1519 nucleotides, covering −277 to −1796 bps upstream start codon of Phaseolus coccineus (subsp. coccineus cultivar Hammond's Dwarf Red Flower) elongation factor whole genome shotgun sequence (QBDZ01192357). pPcEF357 was amplified by PCR using primer pair pPcEF357-F1 and pPcEF357-R1. It was introduced into pEG-A35S-GUS vector to drive GUS gene. Transgenic Arabidopsis seedlings carrying pPcEF357-GUS showed no visible GUS staining in both root and airy parts.

pPsEF817 (SEQ ID NO:456) was used to replace 35S in p7E0-BoG for strong GFR expression, which resulted in p7E7-BoG.’

20.3. Combining GmExo090 with GPR and GFR Cassettes in a Single T-DNA

Constitutive over-expression of GmExo090 (SEQ ID NO:174) in Arabidopsis (Section 9.0) significantly enhanced drought tolerance of transgenic plants. To combine herbicide resistance together with current invention, GFR-GPR (BoG) bi-cassette was cut from p6A0-BoG plasmid and then inserted into pEG-35S-GmExo090 plasmid (Section 9.0) in place of Bar via SacI-SpeI or SacI-EcoRI digestion respectively. These cloning produced construct p6A0-BoG-35S-GmExo090. Promoter pGmUBC990 from soybean ubiquitin-conjugating enzyme (Glyma02g35990, SEQ ID NO:432) was characterized as constitutive moderate promoter. It was amplified from Jack genomic DNA using primers of GmUBC990-Stu1-F (SEQ ID NO:97) and GmUBC990-Xho-R (SEQ ID NO:98). 3′-UTR of soybean Ubiquitin gene Glyma09g02760 (Ubi-T, SEQ ID NO:433) was amplified from Jack using GmUbiT-Xba-F (SEQ ID NO:99) and UbiT-EcoR1Pvu1-R (SEQ ID NO:100) primers. pGmUBC990, GmExo090 and Ubi-T were assembled together in an intermediate cloning vector. pGmUBC990-GmExo090-Ubi-T cassette was then moved into p6A0-BoG or p7CA-BoG vector to have final construct p6A0-BoG-pGmUBC990-GmExo090 or p7CA-BoG-pGmUBC990-GmExo090 respectively.

To mitigate the possible side-effect of constitutive over-expression of GmExo090 on soybean growth and development, promoter RD29A and root specific promoter were used to replace 35S in pEG-35S-GmExo090. RD29A-GmExo090 fragment was amplified by PCR with PvuI attached to N-terminal and BamHI attached to its C-terminal via primers RD29a-PvuI-F (SEQ ID NO:119) and GmExo090 BcIIR (SEQ ID NO:120). This fragment was put into BamHI/PvuI double-digested p6A0-BoG-tGmUbi vector to make construct p6A0-BoG-Rd29A-GmExo090. pVaEF670 (SEQ ID NO:452), pVrEF027 (SEQ ID NO:453), pPsEF774 (SEQ ID NO:454) and pPsEF893 (SEQ ID NO:455) were used to replace pGmEF240 (SEQ ID NO:426) in p6A0-BoG for strong GPR expression, which resulted in p7D0-BoG, p7E0-BoG, p7F0-BoG and p7GO-BoG respectively. pGmUBC990-GmExo090-Ubi-T cassette was put into p7D0-BoG and p7E0-BoG resulting in 2 final constructs for soybean transformation: p7D0-BoG-HP-GmFTB1-GmExo090-2R and p7E0-BoG-HP-GmFTB1-GmExo090-2R.

To analyze the tempo-spatial effects of GmExo090 's over-expression in soybean plants, three root-specific promoters were tested in Arabidopsis and then used to drive GmExo090 expression in soybean. Promoter pGmTIPs (SEQ ID NO:459) contains 1546 nucleotides, covering from −1 to −1546 bps upstream start codon of Glymallg03690. pGmTIPs was amplified by PCR using primer pair pGmTIPs-F1 (SEQ ID NO:476) and pGmTIPs-R1 (SEQ ID NO:477). It was then used to drive GmExo090 expression in pGmTIPs-GmExo090-tGmUbi cassette in GPR-GFR construct of p7E7-BoG-HP-GmFTB1-GmExo090-6R, which is derived from p7E7-BoG-HP-GmFTB1 via Pmel digestion.

Promoter pGmNTT490 (SEQ ID NO:460) contains 1914 nucleotides, covering from −12 to −1925 bps upstream start codon of soybean Glyma.17g124900, which encodes for a nitrate transmembrane transporter. GmNTT490 was amplified by PCR using primer pair pGmNTT490-F1 (SEQ ID NO:478) and pGmNTT490-R1 (SEQ ID NO:479). It was introduced into pEG-A35S-GUS vector to drive GUS gene. Transgenic Arabidopsis seedlings carrying pGmNTT490-GUS cassette have a strong GUS expression in roots and moderate expression in young leaves.

Promoter pGmDOG690 (SEQ ID NO:461) contains 2080 nucleotides, covering from −5 to −2084 bps upstream start codon of soybean Glyma.09g156900, which encodes for a LigB dioxygenase. pGmDOG690 was amplified by PCR using primer pair pGmDOG690-F1 (SEQ ID NO:480) and pGmDOG690-R1 (SEQ ID NO:481). It was introduced into pEG-Δ35S-GUS vector to drive GUS gene. Transgenic Arabidopsis seedlings carrying pGmDOG690-GUS cassette have visible GUS expression in hypocotyls, but not in other parts of the seedlings.

Root-specific pGmTIPs-driven GmExo090-Ubi-T cassette was put into p7E0 and p7E7-BoG respectively, which resulted in two final constructs for soybean transformation: p7E0-BoG-HP-GmFTB1-GmExo090-6R and p7E7-BoG-HP-GmFTB1-GmExo090-6R.

Soybean transformation was performed as described in Section 17.1.

### 21. Overexpression of AtExo970 and its Orthologs Such as GmExo090 can be Combined With Both Pest- and Herbicide Resistance in Crop Plants for Additive Effects

To further facilitate the application of this invention in crop plants for field farming, resistance to common Lepidopteran and Coleopteran insects was further stacked with this invention. Binary construct p7CA-3Bt4-GmExo090 was made to contain 3 Cry expression cassettes in addition to GFR-GPR-GmExo090 tri-cassettes (Section 20) in a single T-DNA fragment for soybean transformation.

### 21.1. Selection of Cry1Ac, Cry1Ca and Cry3Aa for Pest Resistance

Bacilus thuringensis (Bt) has more than 100 subspecies or strains isolated from around world. The crystal endotoxins (Cry) of 5 major strains were commonly used to control different types of insects. There are >770 Cry endotoxins reported so far, which are classified into 74 groups i.e. Cry1 to Cry74. We selected Cry1Ac and Cry1Ca for control of moth larva and Cry3Aa for control of beetles in soybean.

Cry1Ac from strain Kurstaki HD73 is quite conserved with Cry1Aa and Cry1Ab, which have been widely applied in cotton, corn and soybean against Lepidopteran insects. Cry1Ac, in particular, is reported to be effective against soybean looper, budborer, velvetbean caterpillar, cornstalk borer etc.

Cry1Ca from strain Entomocidus was often used in corn, rice and cotton in combination with Cry1A to widen resistance spectrum against Lepidopteran insects. Cry1Ca was reported to be effective against beet armyworm and cotton leaf worm etc.

Cry3Aa from strain San Diego has low toxicity against Lepidopteran insects, but has high insecticidal activity against Coleopteran insects (beetles) such as Colorado potato beetle and western corn root worm etc.

21.2. Stacking Cry1Ac-Cry1Ca-Cry3Aa Tri-Cassette with GmExo090 and Herbicide Resistance in Single T-DNA

Cry proteins from different groups have rather diversified sequences, but their N-terminal core sequence for toxicity has a much conserved 3D structure. Thus, only N-terminal core sequence of Cry1Ac, Cry1Ca and Cry3Aa was used for their expression in soybean.

The protein sequence of Cry1Ac (SEQ ID NO:434, GenBank M11068), Cry1Ca (SEQ ID NO:435, GenBank: X07518.1) and Cry3Aa (SEQ ID NO:436; GenBank: AAA22336) was back-translated based on codon usage in soybean and corn. The codon-optimized nucleotide sequences of Cry1Ac-NT (SEQ ID NO:437), Cry1Ca-NT (SEQ ID NO:438) and Cry3Aa-NT (SEQ ID NO:439) were joined together as a single open frame using 2Ak or 2Ao linkers, which resulted in Cry1AC3A-NT (SEQ ID NO:443). 5′ and 3′ UTRs (SEQ ID NO:440; SEQ ID NO:441) of soybean Rubisco small subunit gene were added to 5′ or 3′ ends of Cry1AC3A-NT for optimal expression of Cry1AC3A (SEQ ID NO:442). Cry1AC3A-NT was synthesized and then cloned into pEarleyGate under 35S promoter, which resulted in construct pEG-35S-Synth1.

Individual Cry1Ac, Cry1Ca or Cry3Aa expression cassettes were made under control of different soybean promoters. Three endogenous promoters of pGmEF630 (SEQ ID NO:444), pGmEF110 (SEQ ID NO:445) and PGmAct7 (SEQ ID NO: 446) were amplified by PCR from soybean Jack genomic DNA with corresponding primer pairs of GmEF630-SaI1-F/GmEF630-Xba1-R (SEQ ID NO:101/SEQ ID NO:102), GmEF110-SaI1-F/GmEF110-Spe1-R (SEQ ID NO:103/SEQ ID NO:104) and GmAct7-SaI1-F/GmAct7-Spe1-R (SEQ ID NO:105/SEQ ID NO:106) respectively. Soybean terminators of tGmHS69 (SEQ ID NO:447), tGmH53 (SEQ ID NO:448) and tGmHS70 (SEQ ID NO: 449) were amplified by PCR from Jack genomic DNA with corresponding primer pairs of tGmHS69-Xho-F/tGmHS69-SaI1-R (SEQ ID NO:107/SEQ ID NO:108), tGmH53-SaI1-F/tGmH53-Xho1-R (SEQ ID NO:109/SEQ ID NO:110), and tGmHS70-SaI1-F/tGmHS70-Xho1-R (SEQ ID NO:111/SEQ ID NO:112) respectively. Cry1Ac-NT (SEQ ID NO:437), Cry1Ca-NT (SEQ ID NO:438) and Cry3Aa-NT (SEQ ID NO: 439) were amplified by PCR from the synthesized template (SEQ ID NO:443) with corresponding primer pairs of Cry1Ac-Xba-F/Cry1Ac-Xho-R (SEQ ID NO:113/SEQ ID NO:114), Cry1Ca-Xba-F/Cry1Ca-Xho-R (SEQ ID NO:115/SEQ ID NO:116), and Cry3Aa-Xba-F/Cry3Aa-Xho-R (SEQ ID NO:117/SEQ ID NO:118) respectively.

Cry1Ac expression cassette (pGmEF630-Cry1Ac-tGmHS69) was made by assembling promoter pGmEF630, Cry1Ac and terminator tGmHS690 together via XbaI and XhoI digestions in a cloning vector. Cry1Ca expression cassette (pGmEF110-Cry1Ca-tGmH53) was made by assembling promoter pGmEF110, Cry1Ca and terminator tGmH53 together via XbaI (SpeI) and XhoI (SaII) digestions. Cry3Aa expression cassette (pGmAct7-Cry3Aa-tGmHS70) was made by assembling promoter pGmAct7, Cry3Aa and terminator tGmHS70 together via XbaI (SpeI) and XhoI (SaII) digestions.

These 3 cassettes were introduced sequentially into p7CA-BoG-pGmUBC990-GmExo090 (Section 20.3) at Sall site adjacent to left border of T-DNA via SaII-XhoI double digestion. pGmAct7-Cry3Aa-tGmHS70 cassette was first cloned in, followed by pGmEF110-cry1Ca-tGmH53, and pGmEF630-Cry1Ac-tGmHS69. The final construct is p7CA-3Bt4-GmExo090, of which the T-DNA has Cry1Ac-Cry1Ca-Cry3Aa tri-cassette in addition to GFR-GPR-GmExo090 tri-cassette in p7CA backbone.

Another tri-Cry bloc was made to contain individual cassette of pPsEF774 (SEQ ID NO:454) -driven Cry1Ac-tPs774 (SEQ ID NO:450), pGmEF110 (SEQ ID NO:445)-driven Cry1Ca-tVr027 (SEQ ID NO:451) and pVaEF670 (SEQ ID NO:452)-driven Cry3Aa-tGmHS70 (SEQ ID NO:449). This tri-Cry bloc was cloned into p7E7-BoG-HP-GmFTB1-GmExo090-6R to have the final construct of p7E7-3Bt-BoG-HP-GmFTB1-GmExo090-6R for soybean transformation.

Co-expression of Cry1Ac, Cry1Ca and Cry3Aa cassettes in a single T-DNA locus provided not only a wide spectrum of resistance against Lepidopteran insects including those tolerant to Cry1Ac but also a parallel resistance against Coleopteran beetle pests in soybean.

Soybean transformation was performed as described in Section 17.1.

22. Identification of AtExo970 Homologs from Other Crop and Vegetable Species

There are 128 Archaeplastida species including economically important vegetables, ornamental flowers, crops and trees, of which whole genome has been sequenced and available to public (https://phytozome.jgi.doe.gov). Potential coding sequences in those genomes were also assembled and annotated. Homologs of AtExo970 from 6 species such as canola (Section 8), soybean (Section 9), corn (Section 11), wheat (Section 12), Brachypodium (Section 13) and rice (Section 15) have been described in previous Examples respectively. Blast search of AtExo970 protein against annotated whole genome sequence of other 32 species (https://phytozome.jgi.doe.gov) allows us to identify the protein homologs of AtExo970 and its coding cDNA and genomic fragment (gDNA) (Seq ID NO:211 to Seq ID NO:410) from each of them. The selected species are Brassica oleracea, Brassica rapa, Cotton (Gossypium hirsutum), Cotton (Gossypium raimondii), Barley (Hordeum vulgare), Millet (Setaria italica), Sorghum (Sorghum bicolor), Miscanthus sinensis, Switchgrass (Panicum virgatum), Tomato (Solanum lycopersicum), Cucumber (Cucumis sativus), Lettuce (Lactuca sativa), Cowpea (Vigna nguiculata), Common bean (Phaseolus vulgaris), Chickpea (Cicer arietinum), Carrot (Daucus carota), Asparagus (Asparagus fficinalis), Potato (Solanum tuberosum), Papaya (Carica papaya), Quinoa (Chenopodium quinoa), Apple (Malus omestica), Orange (Citrus sinensis), Grape (Vitis vinifera), Cassava (Manihot esculenta), Cocoa (Theobroma cacao), Coffea (Coffea arabica), Tea (Camellia sinesis), Olive (Olea europaea), Poplar (Populus trichocarpa), Russian Dandelion (Taraxacum kok-saghyz), Sunflower (Helianthus annuus) and Petunia axillaris (Table 32)

TABLE 31
Oligo-nucleotide Sequence for Gene Amplification and Analysis.

| SEQID NO | Sequence Name | Nucleotides | Length |
|---|---|---|---|
| 1 | AD1 | INGTCGASWGANAWGAA | 16 |
| 2 | AD2 | TGWGNAGSANCASAGA | 16 |
| 3 | AD3 | AGWGNAGWANCAWAGG | 16 |
| 4 | AD4 | STTGNTASTNCTNTGC | 16 |
| 5 | AD5 | NTCGASTWTSGWGTT | 15 |
| 6 | AD6 | WGTGNAGWANCANAGA | 16 |
| 7 | pSK-35S-F1 | TCCAACCACGTCTTCAAAGCAAGTGGA | 27 |
| 8 | pSK-35S-F2 | TCAAGATGCCTCTACCGACAGTGGT | 25 |
| 9 | pSK-35S-F3 | TCTGTCACTTCATCGAAAGGACAGT | 25 |
| 10 | pSK-35S-F4 | ACATGGTGGAGCACGACACTCTCGT | 25 |
| 11 | pSK-OCT-R1 | TGACCATCATACTCATTGCTGATCCATG | 28 |
| 12 | pSK-OCT-R2 | TGGACGTGAATGTAGACACGTCGAA | 25 |
| 13 | pSK-OCT-R3 | AGGTCAAACCTTGACAGTGACGACA | 25 |
| 14 | pSK-OCT-R4 | TGGGTGAGATTCCTTGAAGTTGAG | 24 |
| 15 | D200A960-qF1 | TGAGGAGAGTCCTGAGCTTGGTGTGGT | 27 |
| 16 | D200A960-qR1 | ACGTTCAAGAACAGGCACAGCGTCA | 25 |
| 17 | D200A968-qF1 | TCCTCGTATCCAAGTGTGTTGCAGT | 25 |
| 18 | D200A968-qR1 | TGGCTAGATTATACGAGATGATAGAGGA | 28 |
| 19 | D200A970-qF1 | ACCGATCATCAATGGAGTCTTCGGA | 25 |
| 20 | D200A970-qF2 | TCGACTACACGTCATCACGGTCTCCA | 26 |
| 21 | D200A970-qR1 | AAGAGATCTTCATGTGCTCCACCA | 24 |
| 22 | D200A970-qR2 | TCGTCTCGTACCTATAGCTAGTCAC | 25 |
| 23 | D200A970-qF3 | TTCGGAAACTCTAAGGAACAAGTGTG | 26 |
| 24 | D200A970-qR3 | TTGGCAATGGCCCTATGAGATG | 22 |
| 25 | D200A980-qF1 | TCTCGGAGATTAGACGTCGTGACT | 24 |
| 26 | D200A980-qF2 | TGGTAATGGATTCATCGGAGTAGAC | 25 |
| 27 | D200A980-qR1 | TGTGTCCTCCTTCACACCAAGCA | 23 |
| 28 | D200A980-qR2 | ACTATGAGGATACAAGGCATCCTGA | 25 |
| 29 | D200A990-F1 | ATCATACTCGCACTCATTAGTCAT | 24 |
| 30 | D200A990-R1 | TGTTGTAGTAATTGTTGCGTAGGCT | 25 |
| 31 | AtP2A3qF3 | AGTGACTTGGTTGAGCATTTCACTCCTCTG | 30 |
| 32 | AtP2A3qR3 | AGCTGACCATATATTGATCTTAGCTCCGTCT | 31 |
| 33 | P970-EcoRF | TAGAATTCAACTACTTGATGTGGTTTACGCCAT | 33 |
| 34 | P970-XhoR | AACTCGAGTGTACGTATCAGACACTTGCAAGCA | 33 |
| 35 | AtExo970-XmaF | TACCCGGGATGGATTACCGATCATCAATGGAGTC | 34 |
| 36 | AtExo970-BamR | TTGGATCCTTAAGCTAGTGAGTCTAAGCACCA | 32 |
| 37 | AtExo970-SeqR1 | TGAGAGAGTTGCTCAGCTTGCTTG | 24 |
| 38 | D200A970-qR4 | AATGAACATCATACCCGAGATAGG | 24 |
| 39 | AtExo970-SalF2 | AAGTCGACTGCTTGCAAGTGTCTGATACGTAC | 32 |
| 40 | AtExo970-XbaR2 | AATCTAGACCAATGTTATTCGACATTACACTAC | 33 |
| 41 | AtExo310-BglF2 | TTAGATCTGATTAACGCAACATATTTGAGGT | 31 |
| 42 | AtExo310-XbaR1 | AATCTAGAATGCCAAATTACCACCACAATCAC | 32 |
| 43 | BnExo317-XhoF1 | AACTCGAGACTTCTCCCCACTCTTAAATTGTTGAG | 35 |
| 44 | BnExo317-XbaR1 | AATCTAGAAATCAGAAGTAATGCTTATTCGACT | 33 |
| 45 | AtRd29A-Eco1F | ATGAATTCTGCATGTGACATTTAGACCTTATCGGA | 35 |
| 46 | AtRd29A-XhoR1 | TTCTCGAGAAGTAATCAAACCCTTTATTCCTG | 32 |
| 47 | AtExo970-SalF1 | AAGTCGACAATACCTTCTCACACCTTCTCCA | 31 |
| 48 | AtExo970-XbaR1 | AATCTAGAGCATCTCACACAACACACACTCGAA | 33 |
| 49 | GmExo090-XhoF2 | AACTCGAGTGCAAGTTGGAGCAATAGTACCTC | 32 |
| 50 | GmExo090-XbaR2 | AATCTAGATGCATACTAGATATTTGTATCGGT | 32 |
| 51 | GmExo740-XhoF1 | ATCTCGAGAACAAGATATGCTGAATTGGAGCA | 32 |
| 52 | GmExo740-XbaR1 | TATCTAGATATGCACAGAGAATATCATTGCA | 31 |
| 53 | ZmExo846-XhoF1 | ATCGCCAGCCTCGAGATCGATCTCTCTCAA | 30 |
| 54 | ZmExo846-XbaF1 | AATCTAGACGATCTCTCTCAAGGGACCTTGCT | 32 |
| 55 | ZmExo846-XbaR1 | AATCTAGATATTGCTTCTTATACAGATCTAAGGCA | 35 |
| 56 | BdEx60-XhoF1 | AACTCGAGGTTTCAGAGTTCAGAGAGCTTGATCG | 34 |
| 57 | BdExo960-XbaF1 | AATCTAGAGGTTTCAGAGTTCAGAGAGCTTGATCG | 35 |
| 58 | BdEx60-XbaR1 | ACTCTAGATCACCGATATGATACTCAATCC | 30 |
| 59 | BdExo960-BglR1 | ATAGATCTCACCGATATGATACTCAATCCCACCA | 34 |
| 60 | TaExo220-BglF1 | TCTGAGAGATCTTGATCGACCTGC | 24 |
| 61 | TaExo220-XbaF1 | AATCTAGACTTGATCGACCTGCAGGAAGAATCTG | 34 |
| 62 | TaExo220-XbaR1 | AATCTAGATGCCTTGTTCTCTTTAGCTCATCATC | 34 |
| 63 | TaExo220-BglR1 | ATAGATCTGCCTTGTTCTCTTTAGCTCATCATC | 33 |
| 64 | AtExo970-SalF2 | AAGTCGACTGCTTGCAAGTGTCTGATACGTAC | 32 |
| 65 | AtExo970-XbaR2 | AATCTAGACCAATGTTATTCGACATTACACTAC | 33 |
| 66 | OsExo920-XbF1 | TATCTAGACCTTGGTCGACCGATCAGTAAGAGCA | 34 |
| 67 | OsExo920-XmR1 | ATCCCGGGTCAATTCCACTATAATCAAGTCCCAAG | 35 |
| 68 | gRNA1 | GCGTTTGCAAGAAACATTGC | 20 |
| 69 | gRNA2 | AGCTATAGGTACGAGACGAC | 20 |
| 70 | gRNA3 | ATTCAAGAGTTTCTTTGTAA | 20 |
| 71 | gRNA4 | TACTTAACCCAAGCCTATCT | 20 |
| 72 | GmExo090g1 | AGACTTGGACTTGAGGTCAA | 20 |
| 73 | oGmExo090g1-F | GATTAGACTTGGACTTGAGGTCAA | 24 |
| 74 | oGmExo090g1-R | AAACTTGACCTCAAGTCCAAGTCT | 24 |
| 75 | GmExo090HDR-F | AAAGGTACCCGTGACAGCCGAAGTCAATG | 29 |
| 76 | GmExo090HDR-R | AAAGAATTCTGTGCTCCACAAGATGCTCC | 29 |
| 77 | Exo090Rd29A5Lnk-F | TCACCCCACTCCTACCTTTGCGAATATTTTGTATGTTCAGTG | 42 |
| 78 | Exo090Rd29A5Lnk-R | CACTGAACATACAAAATATTCGCAAAGGTAGGAGTGGGGTGA | 42 |
| 79 | Exo090Rd29A3Lnk-F | GGGTTTGATTACTTCTATTGGAAAACCTCAAGTCCAAGTCTTTTC | 45 |
| 80 | Exo090Rd29A3Lnk-R | GAAAAGACTTGGACTTGAGGTTTTCCAATAGAAGTAATCAAACCC | 45 |
| 81 | Exo090UBC9905Lnk-F | tcaccccactcctacctttgGGCCTTTGTTTATGCTAAGTG | 41 |
| 82 | Exo090UBC9905Lnk-R | CACTTAGCATAAACAAAGGCCcaaaggtaggagtggggtga | 41 |
| 83 | Exo090UBC9903Lnk-F | ATCCTTCCTTCGCTCTCTCCacctcaagtccaagtcttttc | 41 |
| 84 | Exo090UBC9903Lnk-R | gaaaagacttggacttgaggtGGAGAGAGCGAAGGAAGGAT | 41 |
| 85 | GmEF240-Sal1-F | aaaGTCGACTTTCATGGTCCGTAGACTTTT | 30 |
| 86 | GmEF240-EcoR1-R | aaaGAATTCGATGACTTAAACTGGAATCAATTC | 33 |
| 87 | PvEF2-StuI-F | cctAGAATTTTACTTAAATCTCTACTAC | 28 |
| 88 | PvEF2-XhoI-R | aaaCTCGAGAACTGCGTTTGAGTATCTGC | 29 |
| 89 | tGmTub-AvrII-F | aaaCCTAGGaatctacttcagattcttgttc | 31 |
| 90 | tGmTub-SpeI-R | aatACTAGTaagaattttaaaaatctcgtttc | 32 |
| 91 | BarOp-EcoR1-F | aaaGAATTCaacaatgagcccagaaagaagacc | 33 |
| 92 | BarOp-BstBI-R | aaaTTCGAAtcaaatctcagtaactggaagaac | 33 |
| 93 | GmRibT-BstBI-F | aaaTTCGAAGGTTTGAGTTTGAGTTCACA | 29 |
| 94 | GmRibT-StuI-R | aaaAGGCCTTAATTATTATATAATCTTGTAT | 31 |
| 95 | BcpGR-XhoI-F | aaaCTCGAGtatcatcttctctctctctttctg | 33 |
| 96 | BcpGR-AvrII-R | aaaCCTAGGtacacattcatttctctcaatcg | 32 |
| 97 | GmUBC990-StuI-F | aaaAGGCCTGGCCTTTGTTTATGCTAAGT | 29 |
| 98 | GmUBC990-Xho-R | ataCTCGAGGATGGGTTGCGCAAGGTTT | 28 |
| 99 | GmUbiT-Xba-F | aaaTCTAGAGATTGTTTTCCATTTGTTTCTG | 31 |
| 100 | UbiT-EcoR1Pvu1-R | aaaGAATTCGATCGCTAACATTAAGTTATCGATAAAGT | 38 |
| 101 | GmEF630-Sal1-F | aaaGTCGACTACAAGCCGCACAATACAAGTCG | 32 |
| 102 | GmEF630-XbaI-R | aaaTCTAGATCCTTAAATCTGCAAAGAAACTAAAAAAGTTG | 41 |
| 103 | GmEF110-Sal1-F | aaaGTCGACTGAGTCATCAAATGCTTGGGCT | 31 |
| 104 | GmEF110-SpeI-R | ataACTAGTCCAACAAAAGTCAAAGCTGAA | 30 |
| 105 | GmAct7-Sal1-F | aaaGTCGACCTAGTTAATTACACAAATTTTAATTCAtCTAGT | 42 |
| 106 | GmAct7-SpeI-R | aaaACTAGTCTTTTACCAAACTACTGTATGC | 31 |
| 107 | tGmHS69-Xho-F | aaaCTCGAGATCTTTAATTGGTTGTTGCTGTCT | 33 |
| 108 | tGmHS69-Sal1-R | aaaGTCGACTGAGAGATTTAGAAAATCATATCG | 33 |
| 109 | tGmH53-Sal1-F | aaaGTCGACAGAATTTAACCCTTCTTTTCTG | 31 |
| 110 | tGmH53-XhoI-R | aaaCTCGAGTATTGTGACAATTCAAATTTATCTTTAAAT | 39 |
| 111 | tGmHS70-Sal1-F | aaaGTCGACACTTGTTGTCGCTGAAAATCCT | 31 |
| 112 | tGmHS70-XhoI-R | aaaCTCGAGAAACAATATAATCAAATTCAATTACTGG | 37 |
| 113 | Cry1Ac-Xba-F | aaaTCTAGAatggataacaacccaaacatc | 30 |
| 114 | Cry1Ac-Xho-R | aaaCTCGAGtcaagcggtcactggaatgaact | 32 |
| 115 | Cry1Ca-Xba- F | aaaTCTAGAatggaggagaacaaccagaa | 29 |
| 116 | Cry1Ca-Xho-R | aaaCTCGAGtcaaatgatctcaatcttatcaatgtag | 37 |
| 117 | Cry3Aa-Xba-F | aaaTCTAGAatgaacccaaacaacaggtc | 29 |
| 118 | Cry3Aa-Xho-R | aaaCTCGAGtcagttcactggaatgaactc | 30 |
| 119 | RD29a-PvuI-F | aaaCGATCGcgaatattttgtatgttcagtg | 32 |
| 120 | GmExo090BclIR | aaaTGATCAtcaatacaaggaatccaagc | 29 |

TABLE 32
Nucleotide or Protein Sequence of Genetic Elements

| Species/Class | SEQ ID NO | Reference | Type | Length |
|---|---|---|---|---|
| Arabidopsis thaliana | 121 | D200_Insert_Border | NT | 100 |
| | 122 | AtExo970_cDNA | NT | 1417 |
| | 123 | AtExo970_CDS | NT | 1074 |
| | 124 | AtExo970 | AA | 357 |
| | 125 | H-NOX | AA | 35 |
| | 126 | AtExo970_5UTR | NT | 127 |
| | 127 | AtExo970_3UTR | NT | 216 |
| | 128 | AtExo970_Promoter | NT | 708 |
| | 129 | AtExo970_gDNA | NT | 2065 |
| | 130 | AtExo310 | AA | 357 |
| | 131 | AtExo310_cDNA | NT | 1349 |
| | 132 | AtExo310_gDNA | NT | 3045 |
| Canola | 133 | BnExo317 | AA | 357 |
| (Brassica napus) | 134 | BnExo317_cDNA | NT | 1453 |
| | 135 | BnExo317_gDNA | NT | 2507 |
| | 136 | BnExo170 | AA | 357 |
| | 137 | BnExo170_cDNA | NT | 1376 |
| | 138 | BnExo170_gDNA | NT | 2204 |
| | 139 | BnExo140 | AA | 356 |
| | 140 | BnExo140_cDNA | NT | 1331 |
| | 141 | BnExo140_gDNA | NT | 3287 |
| | 142 | BnExo180 | AA | 357 |
| | 143 | BnExo180_cDNA | NT | 1074 |
| | 144 | BnExo180_gDNA | NT | 2491 |
| | 145 | BnExo840 | AA | 357 |
| | 146 | BnExo840_cDNA | NT | 1355 |
| | 147 | BnExo840_gDNA | NT | 3507 |
| | 148 | BnExo580 | AA | 357 |
| | 149 | BnExo580_cDNA | NT | 1151 |
| | 150 | BnExo580_gDNA | NT | 2248 |
| Brassica oleracea | 151 | BoExo202 | AA | 357 |
| | 152 | BoExo202_cDNA | NT | 1074 |
| | 153 | BoExo202_gDNA | NT | 2204 |
| | 154 | BoExo942 | AA | 351 |
| | 155 | BoExo942_cDNA | NT | 1056 |
| | 156 | BoExo942_gDNA | NT | 2203 |
| | 157 | BoExo756 | AA | 321 |
| | 158 | BoExo756_cDNA | NT | 966 |
| | 159 | BoExo756_gDNA | NT | 3108 |
| Brassica rapa | 160 | BrExo169 | AA | 357 |
| | 161 | BrExo169_cDNA | NT | 1392 |
| | 162 | BrExo169_gDNA | NT | 2542 |
| | 163 | BrExo302 | AA | 357 |
| | 164 | BrExo302_cDNA | NT | 1074 |
| | 165 | BrExo302_gDNA | NT | 2500 |
| | 166 | BrExo319 | AA | 356 |
| | 167 | BrExo319_cDNA | NT | 1071 |
| | 168 | BrExo319_gDNA | NT | 3288 |
| | 169 | BrExo233 | AA | 357 |
| | 170 | BrExo233_cDNA | NT | 1144 |
| | 171 | BrExo233_gDNA | NT | 4394 |
| Soybean | 172 | GmExo090 | AA | 354 |
| (Glycine max) | 173 | GmExo090_cDNA | NT | 1384 |
| | 174 | GmExo090_clonned | NT | 1298 |
| | 175 | GmExo090_gDNA | NT | 2704 |
| | 176 | GmExo690 | AA | 366 |
| | 177 | GmExo690_cDNA | NT | 1414 |
| | 178 | GmExo690_gDNA | NT | 3320 |
| | 179 | GmExo740 | AA | 366 |
| | 180 | GmExo740_cDNA | NT | 1693 |
| | 181 | GmExo740_gDNA | NT | 3759 |
| | 182 | GmExo740_clonned | NT | 1334 |
| Corn | 183 | ZmExo846 | AA | 336 |
| (Zea may) | 184 | ZmExo846_CDNA | NT | 1330 |
| | 185 | ZmExo846_gDNA | NT | 2410 |
| | 186 | ZmExo807 | AA | 350 |
| | 187 | ZmExo807_cDNA | NT | 1421 |
| | 188 | ZmExo807_gDNA | NT | 1890 |
| Brachypodium | 189 | BdExo960 | AA | 336 |
| distachyon | 190 | BdExo960_cDNA | NT | 1503 |
| | 191 | BdExo960_gDNA | NT | 2339 |
| | 192 | BdExo960_clonned | NT | 1207 |
| Wheat | 193 | TaExo220 | AA | 335 |
| (Triticum aestivum) | 194 | TaExo220_cDNA | NT | 1246 |
| | 195 | TaExo220_gDNA | NT | 1933 |
| | 196 | TaExo220_gClonned | NT | 1780 |
| | 197 | TaExo220_Clonned | AA | 329 |
| | 198 | TaExo220_cClonned | NT | 1002 |
| | 199 | TaExo960 | AA | 354 |
| | 200 | TaExo960_cDNA | NT | 1270 |
| | 201 | TaExo960_gDNA | NT | 2363 |
| | 202 | TaExo506 | AA | 334 |
| | 203 | TaExo506_cDNA | NT | 1234 |
| Rice | 204 | OsExo920 | AA | 336 |
| (Oryza sativa) | 205 | OsExo920_cDNA | NT | 1011 |
| | 206 | OsExo920_gDNA | NT | 2542 |
| | 207 | OsExo770 | AA | 334 |
| | 208 | OsExo770_cDNA | NT | 1005 |
| | 209 | OsExo770_gDNA | NT | 1619 |
| Arabidopsis thaliana | 210 | AtRd29A_Promoter | NT | 1172 |
| Cotton | 211 | GhExo400 | AA | 351 |
| (Gossypium hirsutum) | 212 | GhExo400_cDNA | NT | 1129 |
| | 213 | GhExo400_gDNA | NT | 2549 |
| | 214 | GhExo800 | AA | 340 |
| | 215 | GhExo800_cDNA | NT | 1023 |
| | 216 | GhExo800_gDNA | NT | 2324 |
| | 217 | GhExo100 | AA | 381 |
| | 218 | GhExo100_cDNA | NT | 1146 |
| | 219 | GhExo100_gDNA | NT | 3597 |
| Cotton | 220 | GrExo200 | AA | 351 |
| (Gossypium | 221 | GrExo200_cDNA | NT | 1056 |
| raimondii) | 222 | GrExo200_gDNA | NT | 2412 |
| | 223 | GrExo600 | AA | 351 |
| | 224 | GrExo600_cDNA | NT | 1056 |
| | 225 | GrExo600_gDNA | NT | 2384 |
| | 226 | GrExo100 | AA | 339 |
| | 227 | GrExo100_cDNA | NT | 1620 |
| | 228 | GrExo100_gDNA | NT | 2742 |
| Barley | 229 | HvuExo934 | AA | 335 |
| ( Hordeum vulgare) | 230 | HvuExo934_cDNA | NT | 1244 |
| | 231 | HvuExo934_gDNA | NT | 1979 |
| | 232 | HvuExo314 | AA | 342 |
| | 233 | HvuExo314_cDNA | NT | 1143 |
| | 234 | HvuExo314_gDNA | NT | 1368 |
| Millet | 235 | SiExo917 | AA | 336 |
| (Setaria italica) | 236 | SiExo917_cDNA | NT | 1861 |
| | 237 | SiExo917_gDNA | NT | 2853 |
| | 238 | SiExo722 | AA | 342 |
| | 239 | SiExo722_cDNA | NT | 1122 |
| | 240 | SiExo722_gDNA | NT | 1337 |
| Sorghum | 241 | SbExo652 | AA | 337 |
| (Sorghum bicolor) | 242 | SbExo652_cDNA | NT | 1299 |
| | 243 | SbExo652_gDNA | NT | 2399 |
| | 244 | SbExo280 | AA | 361 |
| | 245 | SbExo280_cDNA | NT | 1320 |
| | 246 | SbExo280_gDNA | NT | 2515 |
| Miscanthus sinensis | 247 | MsiExo626 | AA | 335 |
| | 248 | MsiExo626_cDNA | NT | 2192 |
| | 249 | MsiExo626_gDNA | NT | 3368 |
| Switchgrass | 250 | PvExo840 | AA | 335 |
| (Panicum virgatum) | 251 | PvExo840_cDNA | NT | 1340 |
| | 252 | PvExo840_gDNA | NT | 2254 |
| | 253 | PvExo794 | AA | 335 |
| | 254 | PvExo794_cDNA | NT | 1281 |
| | 255 | PvExo794_gDNA | NT | 2192 |
| Tomato | 256 | SlExo685 | AA | 354 |
| (Solanum | 257 | SlExo685_cDNA | NT | 1065 |
| lycopersicum) | 258 | SlExo685_gDNA | NT | 2266 |
| | 259 | SlExo773 | AA | 344 |
| | 260 | SlExo773_cDNA | NT | 1035 |
| | 261 | SlExo773_gDNA | NT | 1860 |
| Cucumber | 262 | CsaExo906 | AA | 335 |
| (Cucumis sativus) | 263 | CsaExo906_cDNA | NT | 1009 |
| | 264 | CsaExo906_gDNA | NT | 4093 |
| | 265 | CsaExo905 | AA | 349 |
| | 266 | CsaExo905_cDNA | NT | 1050 |
| | 267 | CsaExo905_gDNA | NT | 3425 |
| Lettuce | 268 | LsaExo341 | AA | 342 |
| (Lactuca sativa) | 269 | LsaExo341_CDNA | NT | 1139 |
| | 270 | LsaExo341_gDNA | NT | 1797 |
| | 271 | LsaExo501 | AA | 368 |
| | 272 | LsaExo501_cDNA | NT | 1429 |
| | 273 | LsaExo501_gDNA | NT | 3968 |
| Cowpea | 274 | VunExo522 | AA | 324 |
| (Vigna unguiculata) | 275 | VunExo522_cDNA | NT | 1714 |
| | 276 | VunExo522_gDNA | NT | 2469 |
| | 277 | VunExo767 | AA | 376 |
| | 278 | VunExo767_cDNA | NT | 1871 |
| | 279 | VunExo767_gDNA | NT | 4248 |
| Common bean | 280 | PvuExo667 | AA | 348 |
| (Phaseolus vulgaris) | 281 | PvuExo667_cDNA | NT | 1047 |
| | 282 | PvuExo667_gDNA | NT | 2065 |
| | 283 | PvuExo606 | AA | 364 |
| | 284 | PvuExo606_cDNA | NT | 1680 |
| | 285 | PvuExo606_gDNA | NT | 4357 |
| Chickpea | 286 | CariExo974 | AA | 371 |
| (Cicer arietinum) | 287 | CariExo974_cDNA | NT | 1116 |
| | 288 | CariExo974_gDNA | NT | 3022 |
| Carrot | 289 | DcExo204 | AA | 353 |
| (Daucus carota) | 290 | DcExo204_cDNA | NT | 1062 |
| | 291 | DcExo204_gDNA | NT | 1946 |
| | 292 | DcExo043 | AA | 330 |
| | 293 | DcExo043_cDNA | NT | 993 |
| | 294 | DcExo043_gDNA | NT | 1920 |
| Asparagus | 295 | AofExo619 | AA | 241 |
| (Asparagus | 296 | AofExo619_cDNA | NT | 726 |
| officinalis) | 297 | AofExo619_gDNA | NT | 1179 |
| | 298 | AofExo873 | AA | 392 |
| | 299 | AofExo873_cDNA | NT | 1179 |
| | 300 | AofExo873_gDNA | NT | 5965 |
| Potato | 301 | StExo908 | AA | 294 |
| (Solanum tuberosum) | 302 | StExo908_cDNA | NT | 885 |
| | 303 | StExo908_gDNA | NT | 1326 |
| | 304 | StExo299 | AA | 243 |
| | 305 | StExo299_cDNA | NT | 732 |
| | 306 | StExo299_gDNA | NT | 1578 |
| Papaya | 307 | CpExo983 | AA | 349 |
| (Carica papaya) | 308 | CpExo983_cDNA | NT | 1050 |
| | 309 | CpExo983_gDNA | NT | 1896 |
| Quinoa | 310 | CquExo885 | AA | 374 |
| (Chenopodium | 311 | CquExo885_cDNA | NT | 1125 |
| quinoa) | 312 | CquExo885_gDNA | NT | 8980 |
| | 313 | CquExo397 | AA | 298 |
| | 314 | CquExo397_cDNA | NT | 897 |
| | 315 | CquExo397_gDNA | NT | 2292 |
| Apple | 316 | MdoExo563 | AA | 343 |
| (Malus domestica) | 317 | MdoExo563_cDNA | NT | 1032 |
| | 318 | MdoExo563_gDNA | NT | 2332 |
| | 319 | MdoExo873 | AA | 329 |
| | 320 | MdoExo873_cDNA | NT | 990 |
| | 321 | MdoExo873_gDNA | NT | 2143 |
| Orange | 322 | CsExo558 | AA | 354 |
| ( Citrus sinensis) | 323 | CsExo558_cDNA | NT | 1138 |
| | 324 | CsExo558_gDNA | NT | 1790 |
| | 325 | CsExo586 | AA | 353 |
| | 326 | CsExo586_cDNA | NT | 1151 |
| | 327 | CsExo586_gDNA | NT | 2957 |
| | 328 | CsExo938 | AA | 342 |
| | 329 | CsExo938_cDNA | NT | 1118 |
| | 330 | CsExo938_gDNA | NT | 2957 |
| | 331 | PpeExo285 | AA | 354 |
| | 332 | PpeExo285_cDNA | NT | 1330 |
| | 333 | PpeExo285_gDNA | NT | 2177 |
| | 334 | PpeExo452 | AA | 358 |
| | 335 | PpeExo452_cDNA | NT | 2112 |
| | 336 | PpeExo452_gDNA | NT | 3482 |
| Grape | 337 | VvExo476 | AA | 324 |
| (Vitis vinifera) | 338 | VvExo476_cDNA | NT | 1065 |
| | 339 | VvExo476_gDNA | NT | 1714 |
| | 340 | VvExo930 | AA | 341 |
| | 341 | VvExo930_cDNA | NT | 1423 |
| | 342 | VvExo930_gDNA | NT | 8600 |
| Cassava | 343 | MeExo207 | AA | 353 |
| (Manihot esculenta) | 344 | MeExo207_cDNA | NT | 1373 |
| | 345 | MeExo207_gDNA | NT | 2618 |
| | 346 | MeExo168 | AA | 350 |
| | 347 | MeExo168_cDNA | NT | 1289 |
| | 348 | MeExo168_gDNA | NT | 2549 |
| | 349 | MeExo732 | AA | 364 |
| | 350 | MeExo732_cDNA | NT | 1534 |
| | 351 | MeExo732_gDNA | NT | 4700 |
| Cocoa | 352 | TcExo688 | AA | 351 |
| (Theobroma cacao) | 353 | TcExo688_CDNA | NT | 1672 |
| | 354 | TcExo688_gDNA | NT | 4581 |
| | 355 | TcExo453 | AA | 364 |
| | 356 | TcExo453_cDNA | NT | 1522 |
| | 357 | TcExo453_gDNA | NT | 3438 |
| Coffea | 358 | CarExo248 | AA | 350 |
| (Coffea arabica) | 359 | CarExo248_cDNA | NT | 1053 |
| | 360 | CarExo248_gDNA | NT | 1745 |
| | 361 | CarExo637 | AA | 326 |
| | 362 | CarExo637_cDNA | NT | 981 |
| | 363 | CarExo637_gDNA | NT | 1573 |
| | 364 | CarExo658 | AA | 326 |
| | 365 | CarExo658_cDNA | NT | 981 |
| | 366 | CarExo658_gDNA | NT | 1570 |
| Tea | 367 | CsiExo493 | AA | 350 |
| (Camellia sinesis) | 368 | CsiExo493_cDNA | NT | 1337 |
| | 369 | CsiExo617 | AA | 348 |
| | 370 | CsiExo617_cDNA | NT | 1047 |
| | 371 | CsiExo617_gDNA | NT | 1621 |
| Olive | 372 | OeuExo919 | AA | 322 |
| (Olea europaea) | 373 | OeuExo919_cDNA | NT | 969 |
| | 374 | OeuExo919_gDNA | NT | 1448 |
| | 375 | OeuExo061 | AA | 320 |
| | 376 | OeuExo061_cDNA | NT | 963 |
| | 377 | OeuExo061_gDNA | NT | 1553 |
| | 378 | OeuExo751 | AA | 325 |
| | 379 | OeuExo751_cDNA | NT | 978 |
| | 380 | OeuExo751_gDNA | NT | 2491 |
| | 381 | OeuExo749 | AA | 325 |
| | 382 | OeuExo749_cDNA | NT | 978 |
| | 383 | OeuExo749_gDNA | NT | 2492 |
| Poplar | 384 | PtExo733 | AA | 346 |
| (Populus trichocarpa) | 385 | PtExo733_cDNA | NT | 1382 |
| | 386 | PtExo733_gDNA | NT | 2103 |
| | 387 | PtExo157 | AA | 357 |
| | 388 | PtExo157_cDNA | NT | 1457 |
| | 389 | PtExo157_gDNA | NT | 3357 |
| Russian Dandelion | 390 | TksExo465 | AA | 350 |
| (Taraxacum | 391 | TksExo465_cDNA | NT | 1053 |
| kok-saghyz) | 392 | TksExo465_gDNA | NT | 4765 |
| | 393 | TksExo061 | AA | 350 |
| | 394 | TksExo061_cDNA | NT | 1053 |
| | 395 | TksExo061_gDNA | NT | 4040 |
| | 396 | TksExo236 | AA | 350 |
| | 397 | TksExo236_cDNA | NT | 1053 |
| | 398 | TksExo236_gDNA | NT | 4751 |
| | 399 | TksExo535 | AA | 576 |
| | 400 | TksExo535_cDNA | NT | 1731 |
| | 401 | TksExo535_gDNA | NT | 9263 |
| Sunflower | 402 | HanExo211 | AA | 345 |
| (Helianthus annuus) | 403 | HanExo211_cDNA | NT | 1038 |
| | 404 | HanExo211_gDNA | NT | 1582 |
| | 405 | HanExo291 | AA | 323 |
| | 406 | HanExo291_cDNA | NT | 972 |
| | 407 | HanExo291_gDNA | NT | 1507 |
| Petunia axillaris | 408 | PaxExo675 | AA | 356 |
| | 409 | PaxExo675_cDNA | NT | 1202 |
| | 410 | PaxExo675_gDNA | NT | 2480 |
| Arabidopsis thaliana | 411 | GmExo29HDR | NT | 1717 |
| Soybean | 412 | GmExo110HDR | NT | 2092 |
| (Glycine max) | 413 | PGmExo090 | NT | 1901 |
| | 414 | GmAct110 | NT | 1458 |
| | 415 | GmUBC960 | NT | 1490 |
| GPR | 416 | AtBCP-CTP_NT | NT | 186 |
| | 417 | AtBCP-CTP | AA | 63 |
| | 418 | GPR | AA | 518 |
| | 419 | AtBCP-5UTR | NT | 109 |
| | 420 | AtEPSPS-3UTR | NT | 168 |
| | 421 | GPR_NT | NT | 1830 |
| Arabidopsis thaliana | 422 | AtKin960_NT | NT | 2716 |
| | 423 | AtKin960 | AA | 663 |
| GPR | 424 | AtRCA-CTP_NT | NT | 174 |
| | 425 | AtRCA-CTP | AA | 59 |
| | 426 | pGmEF240 | NT | 1953 |
| | 427 | pPVEF2 | NT | 2246 |
| | 428 | Tub-T | NT | 351 |
| GFR | 429 | GFR | NT | 562 |
| | 430 | GFR_AA | AA | 183 |
| | 431 | Rib-T | NT | 325 |
| | 432 | PGmUBC990 | NT | 1384 |
| | 433 | Ubi-T | NT | 267 |
| Bt | 434 | Cry1Ac | AA | 610 |
| | 435 | Cry1Ca | AA | 613 |
| | 436 | Cry3Aa | AA | 644 |
| | 437 | Cry1Ac-NT | NT | 1833 |
| | 438 | Cry1Ca-NT | NT | 1842 |
| | 439 | Cry3Aa-NT | NT | 1935 |
| | 440 | GmUbsc-5UTR | NT | 62 |
| | 441 | GmUbsc-3UTR | NT | 50 |
| | 442 | Cry1AC3A | AA | 1917 |
| | 443 | Cry1AC3A_NT | NT | 5880 |
| | 444 | GmEF630 | NT | 1275 |
| | 445 | GmEF110 | NT | 2032 |
| | 446 | GmAct7 | NT | 2007 |
| | 447 | tGmHS69 | NT | 214 |
| | 448 | tGmH53 | NT | 300 |
| | 449 | tGmHS70 | NT | 343 |
| | 450 | tPs774 | NT | 345 |
| | 451 | tVr027 | NT | 314 |
| Legumes and oligos | 452 | pVaEF670 | NT | 1763 |
| | 453 | pVrEF027 | NT | 1712 |
| | 454 | pPsEF774 | NT | 1562 |
| | 455 | pPsEF893 | NT | 1783 |
| | 456 | pPsEF817 | NT | 1801 |
| | 457 | pVaEF895 | NT | 1749 |
| | 458 | pPcEF357 | NT | 1519 |
| | 459 | pGmTIPs | NT | 1546 |
| | 460 | PGmNTT490 | NT | 1914 |
| | 461 | pGmDOG690 | NT | 2080 |
| | 462 | pVaEF670-F1 | NT | 29 |
| | 463 | pVaEF670-R1 | NT | 32 |
| | 464 | pVrEF027-F1 | NT | 31 |
| | 465 | PVrEF027-R1 | NT | 29 |
| | 466 | pPsEF774-F1 | NT | 29 |
| | 467 | pPsEF774-R1 | NT | 29 |
| | 468 | pPsEF893-F1 | NT | 29 |
| | 469 | pPsEF893-R1 | NT | 31 |
| | 470 | pPsEF817-F1 | NT | 29 |
| | 471 | pPsEF817-R1 | NT | 30 |
| | 472 | pVaEF895-F1 | NT | 29 |
| | 473 | pVaEF895-R1 | NT | 30 |
| | 474 | pPcEF357-F1 | NT | 29 |
| | 475 | pPcEF357-R1 | NT | 28 |
| | 476 | pGmTIPs-F1 | NT | 23 |
| | 477 | pGmTIPs-R1 | NT | 27 |
| | 478 | pGmNTT490-F1 | NT | 36 |
| | 479 | pGmNTT490-R1 | NT | 31 |
| | 480 | pGmDOG690-F1 | NT | 25 |
| | 481 | pGmDOG690-R1 | NT | 31 |
| | 482 | ZmExo846-XbaF1 | NT | 32 |
| | 483 | ZmExo846-BglR1 | NT | 35 |

| Species/Class | SEQ ID NO | Reference | Type | Length |
|---|---|---|---|---|
| Bt | 450 | tPa774 | NT | 345 |
| | 451 | tVr027 | NT | 314 |
| Legumes | 452 | pVaEF670 | NT | 1763 |
| | 453 | PVrEF027 | NT | 1712 |
| | 454 | pPsEF774 | NT | 1562 |
| | 455 | pPsEF893 | NT | 1783 |
| | 456 | pPsEF817 | NT | 1801 |
| | 457 | pVaEF895 | NT | 1749 |
| | 458 | pPcEF357 | NT | 1519 |
| | 459 | pGmTIPS | NT | 1546 |
| | 460 | PGmNTT490 | NT | 1914 |
| | 461 | pGmDOG690 | NT | 2080 |
| | 462 | pVaEF670-F1 | NT | 29 |
| | 463 | pVaEF670-R1 | NT | 32 |
| | 464 | pVrEF027-F1 | NT | 31 |
| | 465 | PVrEF027-R1 | NT | 25 |
| | 466 | pPsEF774-F1 | NT | 29 |
| | 467 | pPsEF774-R1 | NT | 29 |
| | 468 | pPsEF893-F1 | NT | 29 |
| | 469 | pPsEF893-R1 | NT | 30 |
| | 470 | pPsEF817-F1 | NT | 29 |
| | 471 | pPsEF817-R1 | NT | 30 |
| | 472 | PVaEF895-F1 | NT | 29 |
| | 473 | pVaEF895-R1 | NT | 30 |
| | 474 | pPcEF357-F1 | NT | 29 |
| | 475 | pGmEF357-R1 | NT | 28 |
| | 476 | pGmTIPs-F1 | NT | 23 |
| | 477 | pGmTIPs-R1 | NT | 27 |
| | 478 | PGmNTT490-F1 | NT | 36 |
| | 479 | PGmNTT490-R1 | NT | 31 |
| | 480 | pGmDOG690-F1 | NT | 25 |
| | 481 | pGmDOG690-R1 | NT | 31 |

SEQIDNO: 121
CAAGGATTCCCTATATGACCAACGTGTAAAATTTAATATGAAACTATTATTTTTGGTAACTGCTGTTTTTGATAAATATTGTAATCA
ATTGAAGAAATTC
SEQIDNO: 122
ATCTATAAATACCTTCTCACACCTTCTCCATTCTTCACATACTCACACACTCAGCTATCAAGAAGAGAACAAACTCTCTCAACTCT
TTCGTTCTCAAGTATTTGCTTGCAAGTGTCTGATACGTACAATGGATTACCGATCATCAATGGAGTCTTCGGAAACTCTAAGGAA
CAAGTGTGCCGCCTGTTACAGGCAATTCAACAAACTGGAACATTTGGTGGAGCACATGAAGATCTCTTATCATTCGGGTCATGA
ACCTACTTGTGGCGTTTGCAAGAAACATTGCAGGTCTTTTGAGTCCCTCCGAGAACATCTCATAGGGCCATTGCCAAAACAAGA
ATGCAAGAACATTTTCAGCCTTCGTGGATGCAGATTTTGCATGACGATACTCGAAAGCCCGAATTCTCGTAGAATCCATCAAGA
GAGATGCCAATTCTCGAGCGTCAACTCTGGATTGACGACTCGAATGGCAGCTTTAGGCTTAAGAGATAAGGCCATGATCGACTA
CACGTCATCACGGTCTCCAAGAGTGGTTGCACTCTCTTGCAAGATGGTAGGAGGAGGAAGTGACGGGTCGTTGGATCTATGCG
CGAGGGTTTGCATAACGGATGAGAGTGACAACGTTATCTTTCACACGTATGTGAAACCTTCGATGGCCGTGACTAGCTATAGGT
ACGAGACGACAGGGATACGCCCGGAAAATCTAAGGGACGCAATGCCATTAAAACAAGTACAAAGAAAGATTCAAGAGTTTCTTT
GTAATGGAGAACCCATGTGGAAGATTCGTCCAAGAGGTGGAAAAGCGAGGATTCTCGTGGGGCATGGCCTCGATCACGATCTT
GACCGCCTTCAACTTGAATATCCTTCTTCCATGATAAGGGATACTGCGAAATACCCACCGTTGATGAAAACAAGCAAGCTGAGC
AACTCTCTCAAGTACTTAACCCAAGCCTATCTCGGGTATGATGTTCATTTTGGGATACAAGACCCTTATGAAGATTGTGTAGCGA
CGATGAGGCTTTACACGAGAATGAGATATCAGAAACACAAGATTGAAGCTTACCCTTTAGCCGCCGATGCGCAGAACCGTAGCA
ACCAGGTGGCTTGGAGGCAGAGTGAGGCCGAGAGGATGTCTCCTGATGAAATGCTCTCAATCTCTCGTTCCGACTATTACTGC
TGGTGCTTAGACTCACTAGCTTAATTTCTAAACTTATGGGGTTATTTAAGTGGTCTCTTAAAATTAGTAATTCTTCAAACTTTTCCA
GAGATTTTTAATTTAGTAGTGTAATGTCGAATAACATTGGTTATGATTTTGTAGTAGTATCTAAATTTTCGAGTGTGTGTTGTGTGA
GATGCCTAGATTGTTATTGAACTTTGTAATAATGCATTAATCTCAATTATTGATTAATTGACTGCT
SEQIDNO: 123
ATGGATTACCGATCATCAATGGAGTCTTCGGAAACTCTAAGGAACAAGTGTGCCGCCTGTTACAGGCAATTCAACAAACTGGAA
CATTTGGTGGAGCACATGAAGATCTCTTATCATTCGGGTCATGAACCTACTTGTGGCGTTTGCAAGAAACATTGCAGGTCTTTTG
AGTCCCTCCGAGAACATCTCATAGGGCCATTGCCAAAACAAGAATGCAAGAACATTTTCAGCCTTCGTGGATGCAGATTTTGCA
TGACGATACTCGAAAGCCCGAATTCTCGTAGAATCCATCAAGAGAGATGCCAATTCTCGAGCGTCAACTCTGGATTGACGACTC
GAATGGCAGCTTTAGGCTTAAGAGATAAGGCCATGATCGACTACACGTCATCACGGTCTCCAAGAGTGGTTGCACTCTCTTGCA
AGATGGTAGGAGGAGGAAGTGACGGGTCGTTGGATCTATGCGCGAGGGTTTGCATAACGGATGAGAGTGACAACGTTATCTTT
CACACGTATGTGAAACCTTCGATGGCCGTGACTAGCTATAGGTACGAGACGACAGGGATACGCCCGGAAAATCTAAGGGACGC
AATGCCATTAAAACAAGTACAAAGAAAGATTCAAGAGTTTCTTTGTAATGGAGAACCCATGTGGAAGATTCGTCCAAGAGGTGGA
AAAGCGAGGATTCTCGTGGGGCATGGCCTCGATCACGATCTTGACCGCCTTCAACTTGAATATCCTTCTTCCATGATAAGGGAT
ACTGCGAAATACCCACCGTTGATGAAAACAAGCAAGCTGAGCAACTCTCTCAAGTACTTAACCCAAGCCTATCTCGGGTATGAT
GTTCATTTTGGGATACAAGACCCTTATGAAGATTGTGTAGCGACGATGAGGCTTTACACGAGAATGAGATATCAGAAACACAAG
ATTGAAGCTTACCCTTTAGCCGCCGATGCGCAGAACCGTAGCAACCAGGTGGCTTGGAGGCAGAGTGAGGCCGAGAGGATGT
CTCCTGATGAAATGCTCTCAATCTCTCGTTCCGACTATTACTGCTGGTGCTTAGACTCACTAGCTTAA
SEQIDNO: 124
MDYRSSMESSETLRNKCAACYRQFNKLEHLVEHMKISYHSGHEPTCGVCKKHCRSFESLREHLIGPLPKQECKNIFSLRGCRFCMTI
LESPNSRRIHQERCQFSSVNSGLTTRMAALGLRDKAMIDYTSSRSPRVVALSCKMVGGGSDGSLDLCARVCITDESDNVIFHTYVKP
SMAVTSYRYETTGIRPENLRDAMPLKQVQRKIQEFLCNGEPMWKIRPRGGKARILVGHGLDHDLDRLQLEYPSSMIRDTAKYPPLMK
TSKLSNSLKYLTQAYLGYDVHFGIQDPYEDCVATMRLYTRMRYQKHKIEAYPLAADAQNRSNQVAWRQSEAERMSPDEMLSISRSD
YYCWCLDSLA
SEQIDNO: 125
HQERCQFSSVNSGLTTRMAALGLRDKAMIDYTSSR
SEQIDNO: 126
ATCTATAAATACCTTCTCACACCTTCTCCATTCTTCACATACTCACACACTCAGCTATCAAGAAGAGAACAAACTCTCTCAACTCT
TTCGTTCTCAAGTATTTGCTTGCAAGTGTCTGATACGTACA
SEQIDNO: 127
TTTCTAAACTTATGGGGTTATTTAAGTGGTCTCTTAAAATTAGTAATTCTTCAAACTTTTCCAGAGATTTTTAATTTAGTAGTGTAAT
GTCGAATAACATTGGTTATGATTTTGTAGTAGTATCTAAATTTTCGAGTGTGTGTTGTGTGAGATGCCTAGATTGTTATTGAACTT
TGTAATAATGCATTAATCTCAATTATTGATTAATTGACTGCT
SEQIDNO: 128
AACTACTTGATGTGGTTTACGCCATTGTTGGTATTCTTGTGTATATAAAATGATTTTTTTTTCTTAGTACATGTACATACAAACTTC
ATTTTTTCAACAAAAAAACAAATTTTAGTGTATTGAAGGCTTTTTAAAAATTCGAAAATGAAGTGAATGATGTAAGAACATTTTCTA
ATATTTTAGAGATTATTCATAAACCTTTTATTGGTATTTTTGAAAGTTATATAAACAATCGTAATTGCTTGATGGTTTGTATTTTTGA
TCCTTTGTATTTTTATGTGAGCCTTTGTATATTTGTTTTTCTTCCCTACACCTACTTAGTGTTGGCTAGCTAGTTATCTGTGTGAGA
TTTTCTTGTTTATAAACTTGATCGACATCACATTTTCACACACACACGTGCATACAATAATAGCATCTTCGTTATCGTCCAAAACAA
ATGACAACTAAAAATTATGATTCCTTTTACCATGAAAAGCGACCCCATCGCATTAAGGCATCTACTTCAATTTCTGTTCGTCTCTA
ACGGTCATATATGAGAATGGTCAGCTTTGCTTTTCTTCATCCTGCTTTTATGAATATTCATCTATAAATACCTTCTCACACCTTCTC
CATTCTTCACATACTCACACACTCAGCTATCAAGAAGAGAACAAACTCTCTCAACTCTTTCGTTCTCAAGTATTTGCTTGCAAGTG
TCTGATACGTACA
SEQIDNO: 129
TGCTTGCAAGTGTCTGATACGTACAATGGATTACCGATCATCAATGGAGTCTTCGGAAACTCTAAGGTAACTATTTTCATGGCCT
ACGCCTATAATACCAAACCATAATTCCATCAAAAACTCCAAGAGAAGACTAATGGAGCTAGTGGCATGCAATATCATATCCAAAT
TCTTGATTCAAGTGATGAAAAACCAAAACTAAGTTTTATTGATTTGAATGTATATACATGTCAATGTACAGGAACAAGTGTGCCGC
CTGTTACAGGCAATTCAACAAACTGGAACATTTGGTGGAGCACATGAAGATCTCTTATCATTCGGGTCATGAACCTACTTGTGGC
GTTTGCAAGAAACATTGCAGGTCTTTTGAGTCCCTCCGAGAACATCTCATAGGTAAATAATAGCCTCTATAAACTTTTCATATATA
TAGATATATAGCATTTCATATACACACTTTGTATATGAAAGCTTTTATAAATTTTGTTATCTCTATATAGGGCCATTGCCAAAACAA
GAATGCAAGAACATTTTCAGCCTTCGTGGATGCAGATTTTGCATGACGATACTCGAAAGCCCGAATTCTCGTAGAATCCATCAA
GAGAGATGCCAATTCTCGAGCGTCAACTCTGTAATCCTCTTTTTCATATTTATATAATTAAACTTTTAACCTATATGAATCGAACGC
AAGAATATATACTTATTCAAATCATATGATTTTCTCATTTTCAGGGATTGACGACTCGAATGGCAGCTTTAGGCTTAAGAGATAAG
GCCATGATCGACTACACGTCATCACGGTCTCCAAGAGTGGTTGCACTCTCTTGCAAGATGGTAGGAGGAGGAAGTGACGGGTC
GTTGGATCTATGCGCGAGGGTTTGCATAACGGATGAGAGTGACAACGTTATCTTTCACACGTATGTGAAACCTTCGATGGCCGT
GACTAGCTATAGGTACGAGACGACAGGGATACGCCCGGAAAATCTAAGGGACGCAATGCCATTAAAACAAGTACAAAGAAAGA
TTCAAGAGTTTCTTTGTAATGGAGAACCCATGTGGAAGATTCGTCCAAGAGGTGGAAAAGCGAGGATTCTCGTGGGGCATGGC
CTCGATCACGATCTTGACCGCCTTCAACTTGAATATCCTTCTTCCATGATAAGGTTGACATTTATATTTAAGAAAATAAATAAAGA
AAATACATATAACAAGATTCTTTAGTGTTACTTCTCTTTCGATATAACATATGATTTTATGTGAGTTTATATGTTAATGGTGAAGTTT
AATGTTTGTATGTAGGGATACTGCGAAATACCCACCGTTGATGAAAACAAGCAAGCTGAGCAACTCTCTCAAGTACTTAACCCAA
GCCTATCTCGGGTAATAATATTATTTTTTGGTATTATAATATATTGAATCAAATAAACTAATTTGGAAATTGTGTATATATGCATGC
ACTCGTATGTTGATACAATATATGTGTATAAATGATTACTTTTTGATGATGAGGGTATTGATGCTTGTATGGGTTTAAAGTTATAAG
GAAGAGTTTCAAAACGTGAAAAGATTCACTGTTTAATAATACATTAATCTCTATATATATCTACACACATATACATATATGGATTGT
ATATGACTAGCACCAAAATCGTTAAGACATTTTTATTTTACATGAGATGCTTATTATATTCAAGATTATATGTTAATAAATGATGTG
TTTATATAGGTATGATGTTCATTTTGGGATACAAGACCCTTATGAAGATTGTGTAGCGACGATGAGGCTTTACACGAGAATGAGA
TATCAGAAACACAAGATTGAAGCTTACCCTTTAGCCGCCGATGCGCAGAACCGTAGCAACCAGGTGGCTTGGAGGCAGAGTGA
GGCCGAGAGGATGTCTCCTGATGAAATGCTCTCAATCTCTCGTTCCGACTATTACTGCTGGTGCTTAGACTCACTAGCTTAATTT
CTAAACTTATGGGGTTATTTAAGTGGTCTCTTAAAATTAGTAATTCTTCAAACTTTTCCAGAGATTTTTAATTTAGTAGTGTAATGT
CGAATAACATTGGT
SEQIDNO: 130
MDYRLLMDSSETLRNKCGGCYRQFNKKEHLVEHMRISYHSVHEPTCGICNKHCRSFDSLREHLIGPLPKQECKNIFSIRGCRFCLTIL
ESPNARRIHQERCQLSNVTSGLMIRMAALGLRNNSTIDYTSSRSPRVVALSCKMVGGGSDGSLDLCARVCITDESENVVFHTYVKPTI
PVTNYRYEMTGIRPENLRDAMRLKHAQRKVQEFLCNGEPMWKIRPRNGKARILVGHGLDNHLDSLQLEYSSSMIRDTAEYPPLMKSS
KLSNSLKYLTQAYLGYDIHVGIQDPYEDCVATMRLYTRMRYQKHRAEAYPLASDTQNHNNFAAWRQNELERMSPEELLDLSRSDYY
CWCLDSVA
SEQIDNO: 131
CAAAAAAAAACAAAGAAGATTAAACCTGTTTAATCAAATTCTCCTGATTAACGCAACATATTTGAGGTTGCTGTTATTTACTATGG
ACTACAGATTGTTAATGGATTCCTCAGAAACCCTAAGGAACAAGTGTGGAGGGTGTTATAGGCAATTCAACAAGAAGGAGCACT
TGGTGGAACACATGAGGATCTCTTATCATTCGGTTCATGAACCTACTTGTGGTATTTGCAACAAACATTGCCGATCTTTTGACTC
CCTCCGTGAACATCTCATTGGGCCATTGCCGAAACAAGAATGTAAGAACATTTTCAGCATTCGCGGCTGCAGATTCTGTCTTAC
GATCCTCGAAAGCCCCAACGCTCGTAGAATCCATCAAGAGAGATGCCAGCTCTCAAACGTCACTTCTGGATTAATGATTCGTAT
GGCGGCCTTAGGCCTAAGAAACAACTCAACAATTGACTACACTTCTTCGAGGTCACCTCGAGTGGTGGCACTCTCATGCAAGAT
GGTTGGAGGAGGCAGTGACGGATCGCTTGACCTATGCGCAAGAGTTTGCATTACGGATGAGAGCGAAAATGTTGTGTTCCACA
CGTATGTGAAGCCAACGATACCCGTAACGAATTATAGGTATGAGATGACAGGGATTCGACCTGAAAATCTAAGGGACGCAATGC
GATTAAAGCACGCACAGAGAAAGGTTCAAGAGTTTCTTTGTAATGGAGAACCAATGTGGAAGATTCGTCCAAGAAATGGGAAAG
CAAGGATTCTCGTTGGACATGGACTTGATAACCATCTTGACTCTCTTCAACTTGAATATTCTTCCTCTATGATAAGAGATACTGCG
GAATACCCTCCATTGATGAAATCAAGCAAGCTAAGCAACTCTCTCAAGTACTTAACCCAAGCCTATCTCGGTTATGATATTCATG
TGGGAATACAAGATCCTTACGAGGACTGTGTCGCGACAATGAGGCTATACACGAGAATGCGATATCAGAAACACAGGGCCGAG
GCCTATCCGCTGGCCTCGGACACGCAGAACCACAATAACTTTGCGGCGTGGAGGCAGAATGAACTAGAGAGGATGTCTCCAGA
GGAGTTGCTCGACCTTTCACGTTCAGACTATTACTGCTGGTGCTTGGACTCGGTTGCTTGAAAAAGAAAGTTATACTGATGGTG
CTTGATCATCTCCGAAAAATAAGATGCATGCGAGGATATATTTAGTAAAGTATAATTGAAAATCGAATAAACATTATCTTTATTAGT
GATTGTGGTGGTAATTTGGCATTCTTGTATCATCTATGTTACATGTAATTGTAACTCATGCATGGTTATGTACTTCATACGCG
SEQIDNO: 132
ACATATTTGAGGTTGCTGTTATTTACTATGGACTACAGATTGTTAATGGATTCCTCAGAAACCCTAAGGTATTTATCTATATTAGA
GTAAAATCTTGTGTTTCTTTGATATAATTGTACCACCATTGGTGTACGCATTTTTTCTATTACCACCGACATCTAATGATATCGAAT
ATGTATGCATAAAACTCTTTCAAACATGAACAATACGTAATCTTAACTAGGTAAATTTGTAATTTACTAATCTAGTGCGAAACCGTA
TCTAAAATATATATAAAGTATAAATTATTATTAAAAACATATGGATACATGCAGGAACAAGTGTGGAGGGTGTTATAGGCAATTCA
ACAAGAAGGAGCACTTGGTGGAACACATGAGGATCTCTTATCATTCGGTTCATGAACCTACTTGTGGTATTTGCAACAAACATTG
CCGATCTTTTGACTCCCTCCGTGAACATCTCATTGGTATATATATCTCTAACCTAGTTCATAACTTGTCTTTAATATTTCACTTTTC
CTCATTTCATTTTGGGTCATATCGTACATAGGGCCATTGCCGAAACAAGAATGTAAGAACATTTTCAGCATTCGCGGCTGCAGAT
TCTGTCTTACGATCCTCGAAAGCCCCAACGCTCGTAGAATCCATCAAGAGAGATGCCAGCTCTCAAACGTCACTTCTGTATATAT
CATCTTCACATATATGAAATAACCATCGTATACATGTCATAATTATTTCTTCTTCAATGTATATTTAATCTTGCATTTAAACATTACT
AAAATATATATATTAAAATTTAAAATACTAAAAACGACCTTATTTAAAAAATCATTAGTATTAAACGTAAATGCATATAGATAATATA
TAATCTAATATTTTTACTGATATTTTAGGGATTAATGATTCGTATGGCGGCCTTAGGCCTAAGAAACAACTCAACAATTGACTACA
CTTCTTCGAGGTCACCTCGAGTGGTGGCACTCTCATGCAAGATGGTTGGAGGAGGCAGTGACGGATCGCTTGACCTATGCGCA
AGAGTTTGCATTACGGATGAGAGCGAAAATGTTGTGTTCCACACGTATGTGAAGCCAACGATACCCGTAACGAATTATAGGTAT
GAGATGACAGGGATTCGACCTGAAAATCTAAGGGACGCAATGCGATTAAAGCACGCACAGAGAAAGGTTCAAGAGTTTCTTTGT
AATGGAGAACCAATGTGGAAGATTCGTCCAAGAAATGGGAAAGCAAGGATTCTCGTTGGACATGGACTTGATAACCATCTTGAC
TCTCTTCAACTTGAATATTCTTCCTCTATGATAAGGTAAATAAATACTTTTACTAGAAACACTAATTTCTTAAGTTATATAATGTTTG
GTTTTTAGTCTATAATCTATGGTCATAAATAAAAGTGAAAAAAAAAATATGTGATAAGAAAGATATTGACCGTTTTAACTCTTTATC
AAAAGAAATAAAAGATACTGACCATTTTCAACTTTAGTATCATTAATCCTTTCGTGTAAGAACCCTATCGATATACTATTAAATACA
CTATGGATATTGACCGTTTAGTTTTTAAACTAAATACACTATTTTTTGCTGAACTGAATTTCATATAGATGATAAAAAGAAATTGTA
AACCAAGACGATTTTTTTTTTTGTTTTTGTTTTCCTAACCGCTGATTTATATACAATTCATATGTATATAGTGGGAATTTTACAATTA
TATCAATTATTTTTGTTGTTGTTAATAGAGATACTGCGGAATACCCTCCATTGATGAAATCAAGCAAGCTAAGCAACTCTCTCAAG
TACTTAACCCAAGCCTATCTCGGGTAATGTTTTTTTTTTTTTACATTTCCATAGTATACAAACTGACATTGTCATATTGGATATCAT
AAATTATTGAAAATCTATTGATCAGACTTTAAAATGTTCCATGATAATTTAAAAAATATTCAACTTGAAAACAAAATAGTTCATCTAC
TAAACCATGCATATACTTTTCAAAAAAAGAAAACAAATCTTGTGTGATATTCGGACGCAACAACTATAAGCTAGTAAAGAAGGTAT
CCGATTTTTGACATGATTAAAAGTTATTAATCACCGTATAAAGTACGACATGCGTATTAGCTTAAAAAGTTTCTTAAGAATTCGAAT
AAAATCAACCGATGATAATCCTAAGAATATCTCGTCCATATCAAAGTTCGATTTGTCTGTCTCTACTCTCTACCTCATATTTTATAT
TATTGTGATTCATAACCAAAAGTTCGTATGTCTTATTTTGTTCCCCTCCTACCTATAAGGTTTTCCAAAAATACTTTCATACACAAA
TTGCAATTTTAATTTTTCTAAGAAATACAAATCTTTAACTGGGATTAATATGAAAAATAGTCGAATCTTTCAAACTAAAGAAATATA
CTTTTTAGTTTACGTTAGTGTCAATGATTTCGTTTTTAGAGCTAGGTTGAGTTTAACAGGTTTGTTTTAGATTAGAGTTTAGAATTC
ATCAGTTATATTTTAAAATAGTACTTTAACTAAAACAATCTAGAATAATTGCGGTTCTTGTAGTAATTTACTAATTTTCAACTTCCTT
GGCTTTGTAAAGTTATGATATTCATGTGGGAATACAAGATCCTTACGAGGACTGTGTCGCGACAATGAGGCTATACACGAGAAT
GCGATATCAGAAACACAGGGCCGAGGCCTATCCGCTGGCCTCGGACACGCAGAACCACAATAACTTTGCGGCGTGGAGGCAG
AATGAACTAGAGAGGATGTCTCCAGAGGAGTTGCTCGACCTTTCACGTTCAGACTATTACTGCTGGTGCTTGGACTCGGTTGCT
TGAAAAAGAAAGTTATACTGATGGTGCTTGATCATCTCCGAAAAATAAGATGCATGCGAGGATATATTTAGTAAAGTATAATTGAA
AATCGAATAAACATTATCTTTATTAGTGATTGTGGTGGTAATTTGGCATTCTTGTATCATCTATGTTACATGTAATTGTAACTCATG
CATGGTTATGTACTTCATACGCGTCTGTGT
SEQIDNO: 133
MDYRSSMESSETLRNKCAACYRQFNKMEHLVEHMKISYHSGHEPTCGVCKKHCRSFESLREHLIGPLPKQECKNIFSLRGCRFCMM
ILESPNARRIHQERCQFSSVNAGLTTRMAALGLRDKAMIDYTSSRSPKMVALSCKMVGGGSDGSLDLCARVCITDENDNVVFHTYVK
PSMVVTNYRYETTGIRPENLRDAMPLKHAQRKIQEFLCNGEPMWKIRPRGGKGRILVGHGLDHDLDRLQLEYPSSMMRDTAKYPPL
MKTSKLSNSLKYLTQAYLGYDIHVGIQDPYEDCVATMRLYTRMRYQKHKIEAYPLPADVQNRSNQVAWRQSEVERMSPNEMLSISRS
DYYCWCLDSLA
SEQIDNO: 134
GCATAATACATTAATAAAAAAACTCATCAAAAGAATTGTTTAACTTCTCCCCACTCTTAAATTGTTGAGTTCTTTGTTTGCATTTTC
ATACGTACCATGGATTACAGATCATCTATGGAGTCATCGGAAACCCTAAGGAACAAGTGCGCAGCTTGTTATAGGCAATTCAAC
AAAATGGAACATTTGGTGGAACACATGAAGATCTCTTATCACTCCGGTCATGAGCCTACTTGTGGGGTTTGCAAGAAACATTGC
CGATCTTTTGAGTCACTCCGCGAACATCTCATAGGACCATTGCCAAAACAAGAATGCAAGAACATTTTTAGCCTTCGOGGATGC
AGATTCTGCATGATGATCCTCGAAAGCCCTAACGCTCGTAGGATTCATCAAGAGAGATGCCAATTTTCGAGCGTCAATGCTGGA
TTGACGACTCGTATGGCAGCGTTAGGCCTTAGAGATAAAGCCATGATCGACTACACGTCATCACGGTCTCCAAAAATGGTTGCA
CTCTCTTGCAAGATGGTAGGAGGAGGAAGCGACGGGTCGTTGGATCTATGCGCAAGGGTTTGCATAACGGATGAGAACGACAA
CGTTGTGTTCCACACGTACGTGAAACCGTCAATGGTCGTGACGAACTATAGGTACGAGACGACCGGTATACGTCCAGAGAATC
TGAGGGACGCAATGCCGTTGAAACATGCACAAAGAAAGATTCAAGAATTTCTTTGTAATGGAGAACCCATGTGGAAGATTCGTC
CAAGAGGTGGGAAAGGGAGGATTCTCGTGGGACATGGGCTGGATCACGATCTTGACCGCCTTCAACTTGAATATCCTTCTTCC
ATGATGAGGGATACTGCAAAATATCCTCCGTTGATGAAAACAAGCAAGCTGAGCAATTCTCTCAAGTACTTAACCCAAGCCTATC
TCGGGTATGATATTCATGTTGGGATACAAGACCCATACGAAGATTGTGTAGCGACGATGAGGCTTTACACGAGAATGAGATATC
AGAAACATAAGATTGAAGCATATCCTTTACCAGCCGACGTGCAGAACCGTAGCAATCAAGTGGCTTGGAGGCAGAGCGAGGTC
GAAAGGATGTCTCCTAATGAAATGCTCTCCATCTCTCGCTCCGACTACTATTGCTGGTGCTTGGACTCCCTCGCTTAATTCCAAA
AACTATGGGGTTAAAATTTGAGAATCTCTCATAATTACTTCATAAACTTTTTCGAGATTTTTAATTTAGTAGCGTAAAGTCGAATAA
GCATTACTTCTGATTTCAAATAGTATCTAAATTAATTTTATAGTATGTGTCGTGTGAGATGCGAATGTTGTTCCTGTACTTCTAATA
ACGCATATTGCTCTTAATTATTAATAATTATTGTTTTTTAAAAATTAATTAGTAATCACTTGTTAGCTTTTGTGTATAAATATATCTAT
ATTTGTCTTAGAA
SEQIDNO: 135
ATGGATTACAGATCATCTATGGAGTCATCGGAAACCCTAAGGTAACCATTTTCATCCTAAGCGTGTGTTATATCAAGCGTTTTAAT
TGAAAAAAAAATCCCAAAGAAAAACTCAATGGAGTGGCATGCAATATCATACCCAAATTCTGTGGTCAACTGATGAAAAATTAAG
ACTAAAATTATTTTTTTTTCTAAAATATATTTGTATATATATGTTAAAAATATATTGATGTATATATGTACACGTACAGGAACAAGTG
CGCAGCTTGTTATAGGCAATTCAACAAAATGGAACATTTGGTGGAACACATGAAGATCTCTTATCACTCCGGTCATGAGCCTACT
TGTGGGGTTTGCAAGAAACATTGCCGATCTTTTGAGTCACTCCGCGAACATCTCATAGGTAAATAAGAAGCTATATAAAAAGAGT
GACATATAGTTTTATATATACGCTATGCATTTGTATGTTAATTTTTAATTGTGTAATGTCAATAGGACCATTGCCAAAACAAGAATG
CAAGAACATTTTTAGCCTTCGCGGATGCAGATTCTGCATGATGATCCTCGAAAGCCCTAACGCTCGTAGGATTCATCAAGAGAG
ATGCCAATTTTCGAGCGTCAATGCTGTACGTAATCGTATTATTGCCCTTATCTCAATACTCCTCTTCTCATATTTGCGTAACTAAA
CTTGTAAATTGACCACATGAAAAAAATAATTCTTATATAAATAATGTAATATCATATGATTTGTCTTTTTCAGGGATTGACGACTCG
TATGGCAGCGTTAGGCCTTAGAGATAAAGCCATGATCGACTACACGTCATCACGGTCTCCAAAAATGGTTGCACTCTCTTGCAA
GATGGTAGGAGGAGGAAGCGACGGGTCGTTGGATCTATGCGCAAGGGTTTGCATAACGGATGAGAACGACAACGTTGTGTTC
CACACGTACGTGAAACCGTCAATGGTCGTGACGAACTATAGGTACGAGACGACCGGTATACGTCCAGAGAATCTGAGGGACGC
AATGCCGTTGAAACATGCACAAAGAAAGATTCAAGAATTTCTTTGTAATGGAGAACCCATGTGGAAGATTCGTCCAAGAGGTGG
GAAAGGGAGGATTCTCGTGGGACATGGGCTGGATCACGATCTTGACCGCCTTCAACTTGAATATCCTTCTTCCATGATGAGGTG
ACATTTCTATAAAGAAGAAAAGTAAAGAGGACACATATATATAACTAGATTCCTTATAGTGTTACTTGGTTTCTCGCAACACAACC
CAGACTTTACGTGAGTTGTTTTACGATTAACGTGAATTTCTAATATCATTTTTATATATTGGTGTGTGTAGGGATACTGCAAAATAT
CCTCCGTTGATGAAAACAAGCAAGCTGAGCAATTCTCTCAAGTACTTAACCCAAGCCTATCTCGGGTAATATATCTACTTTTGTG
TTTGTAATACAACTTTCGAATATAACATTAATTTGGAAAATGAATATAGATATTTGCATACTTTACGTGTATTGATACATAAGTGTA
TCATGTACTATCCATTAATTTTTTTGGTGATGTATGGGTATTGATGCTTTGTATGGGCTTAAAGCTAAAAGGAAGGGATTCAAAAC
GTGAAAAGATTCATATGTTATATAATATAACCATTTTCTAACAAAAACAAAAAGAATTTACATAGTTGGAGATTTTTAATTTACATG
AGATGCTTACTAGCGTAACTTTTTATCTCCAAGTTGACTAAATCAACCTATTTATTTCATTTTATTTCCTATCAATTACAAATAGTTC
AACTAGTATACCATTGTTTTGTATGTAAAATTATAGGGATGAGTGTGTATGCCTAATATCATATGTTCCATATAATCTGTGTTGGC
ACCGTAGAAGTAATCTAAAACACTTGAGATCAATTAATTATACCCATTATCTAATGTACAACAATATATATATATTAAGGATGGTAT
TGATGTGCTTATAGGTATGATATTCATGTTGGGATACAAGACCCATACGAAGATTGTGTAGCGACGATGAGGCTTTACACGAGA
ATGAGATATCAGAAACATAAGATTGAAGCATATCCTTTACCAGCCGACGTGCAGAACCGTAGCAATCAAGTGGCTTGGAGGCAG
AGCGAGGTCGAAAGGATGTCTCCTAATGAAATGCTCTCCATCTCTCGCTCCGACTACTATTGCTGGTGCTTGGACTCCCTCGCT
TAATTCCAAAAACTATGGGGTTAAAATTTGAGAATCTCTCATAATTACTTCATAAACTTTTTCGAGATTTTTAATTTAGTAGCGTAA
AGTCGAATAAGCATTACTTCTGATTTCAAATAGTATCTAAATTAATTTTATAGTATGTGTCGTGTGAGATGCGAATGTTGTTCCTGT
ACTTCTAATAACGCATATTGCTCTTAATTATTAATAATTATTGTTTTTTAAAAATTAATTAGTAATCACTTGTTAGCTTTTGTGTATAA
ATATATCTATATTTGTCTTAGAA
SEQIDNO: 136
MDCRSSMESSETLRNKCAACYKQFNKLEHLVEHMKISYHSGHEPMCGVCKKHCRSFESLREHLIGPLPKQECKNIFSFRGCRFCLMI
LETPNARRIHQERCQFSSVNAGLTTRMAALGVRDKAMIDYTSSRSPKVVALSCKMVGGGSDGSLDLCARVCITDESDNVVFHTYVKP
SMPVTNYRYEKTGIRPENLRDAMPLKHAQRKIQEFLCNGEPMWKIRPRGGKGRILVGHGLDHDLDRLQLEYPSSMIRDTAKYPPLMK
TSKLSNSLKYLTQAYLGYDVHVGIQDPYEDCVATMRLYTRMRYQKHKIEAYPLAADAHNRSNQVAWRQNEFERMSPDEMLSISRSD
YCCWCLDSLA
SEQIDNO: 137
ACACATCTATAATACAAAAAGGATCATCAAAACAGTTGTTTAAACTCTCTCATCTCTCAAGTTGCTACGTTTTTGTTTGTATTTTCC
GATACGTACCATGGATTGCAGATCATCTATGGAGTCATCGGAAACCCTAAGGAACAAGTGCGCAGCTTGTTATAAACAATTCAA
CAAATTAGAACATTTAGTGGAGCACATGAAGATCTCGTATCACTCCGGTCATGAGCCTATGTGTGGCGTTTGCAAGAAACATTG
CCGATCTTTTGAGTCCCTCCGGGAACATCTCATAGGGCCATTGCCAAAACAAGAATGCAAGAACATTTTCAGCTTTCGCGGATG
CAGATTCTGCTTGATGATCCTCGAAACCCCGAACGCTCGTAGGATCCATCAAGAGAGATGCCAATTTTCGAGCGTCAATGCTGG
ATTGACGACTCGTATGGCGGCCTTAGGCGTAAGAGATAAGGCCATGATCGACTACACGTCGTCTAGATCCCCAAAAGTGGTTG
CACTCTCTTGCAAGATGGTAGGAGGGGGAAGCGACGGGTCGCTGGATCTATGCGCAAGGGTTTGCATAACGGATGAGAGCGA
CAACGTTGTTTTCCATACGTACGTGAAACCGTCAATGCCCGTGACGAACTATAGGTATGAGAAGACCGGCATACGTCCGGAGAA
TCTAAGGGACGCAATGCCGTTGAAACATGCACAAAGAAAGATTCAAGAGTTTCTTTGTAATGGAGAACCCATGTGGAAGATTCG
TCCAAGAGGTGGGAAAGGGAGGATTCTCGTGGGACATGGCCTCGATCACGATCTTGACCGCCTTCAACTTGAATATCCTTCTTC
CATGATAAGGGATACTGCGAAATACCCTCCGTTGATGAAAACAAGCAAGCTGAGCAATTCCCTCAAGTACTTGACCCAAGCCTA
TCTCGGGTATGATGTTCATGTTGGGATACAAGACCCATACGAAGATTGTGTAGCGACGATGAGGCTTTACACGAGAATGAGATA
TCAGAAACACAAGATTGAAGCTTATCCTTTAGCTGCCGACGCGCACAACCGTAGCAATCAAGTGGCTTGGAGGCAGAACGAGT
TCGAGAGGATGTCTCCTGATGAAATGCTTTCCATCTCTCGATCCGACTACTGTTGCTGGTGCTTGGACTCCCTCGCCTGATTTCT
AAAACTATGGGGTTAAGTTGAGGAATTTCACATAATAACTTCGTAAACTTTTTCAAGATATTAAATTTAGTAGCGTAATGTCGAAT
AAGAATCAGTTGTTATTTTTAATATATACTAGTATCTAAATTTTATAGTATGTGTCGTGTGAGACGCATAAATTGTTACTGTACTTG
TAATAATGCATTGCTTTTCAATGATTA
SEQIDNO: 138
ATGGATTGCAGATCATCTATGGAGTCATCGGAAACCCTAAGGTAACCTATCTTCCATCGTACGTGTATTATATTATATCAAGCGT
TTATATGAAAATATCGAAAAGAACTCATGGAGGATGTCATCCAGTATCACACCCAAATTCTATAATCAATTGATGAAAAATAAGAA
ATAAAGTTTTTCTTAATTTATAGTTTTTATACATATGTACACGCACGTGCAGGAACAAGTGCGCAGCTTGTTATAAACAATTCAAC
AAATTGGAACATTTAGTGGAGCACATGAAGATCTCGTATCACTCCGGTCATGAGCCTATGTGTGGCGTTTGCAAGAAACATTGC
CGATCTTTTGAGTCCCTCCGGGAACATCTCATAGGTAAATGATAAGCTATATATATTAAGAGAAATATATCGTTTTATATATTCAC
GACGCATATGTGTATTTATTTTTTATTGTGCTATATTATTAGGGCCATTGCCAAAACAAGAATGCAAGAACATTTTCAGCTTTCGC
GGATGCAGATTCTGCTTGATGATCCTCGAAACCCCGAACGCTCGTAGGATCCATCAAGAGAGATGCCAATTTTCGAGCGTCAAT
GCTGTAATCTTTTCTTCTAATATTTGCGCAAATTAAAATTGTTAATAATCAACCGCATAACAATATATTCTTACATATTTTCTATGTA
TCAGGGATTGACGACTCGTATGGCGGCCTTAGGCGTAAGAGATAAGGCCATGATCGACTACACGTCGTCTAGATCCCCAAAAG
TGGTTGCACTCTCTTGCAAGATGGTAGGAGGGGGAAGCGACGGGTCGCTGGATCTATGCGCAAGGGTTTGCATAACGGATGA
GAGCGACAACGTTGTTTTCCATACGTACGTGAAACCGTCAATGCCCGTGACGAACTATAGGTATGAGAAGACCGGCATACGTC
CGGAGAATCTAAGGGACGCAATGCCCTTGAAACATGCACAAAGAAAGATTCAAGAGTTTCTTTGTAATGGAGAACCCATGTGGA
AGATTCGTCCAAGAGGTGGGAAAGGGAGGATTCTCGTGGGACATGGCCTCGATCACGATCTTGACCGCCTTCAACTTGAATAT
CCTTCTTCCATGATAAGGTGGCATTTCTATAAAGAAAAAAAAAATATAAAGAGGATATATACGTATATAACTAGATTCCTTTAGTG
TTACTTGAAATCTTTCAATATAGCCCATGATTTTATGTGTGTTTTTTATATGACGGGATTTTGTATCATTTTATTGCTGTATGTAGG
GATACTGCGAAATACCCTCCGTTGATGAAAACAAGCAAGCTGAGCAATTCCCTCAAGTACTTGACCCAAGCCTATCTCGGGTAA
TATATCTAATTTTGTTTTTGTAATAATATTTTCGAACCTAACACATTAATTTGGAAAATGATTATAGATGCATGCATACATTACCTAT
ATTGATACACATATATATCATAATAGGTGGGTATTGATGTTTGTATGGGCTTAAATGGGAGGGCTTCAAAACGTAAGATTTACAT
GTTTTCAAATATATTCATTTCAAAATTTACACATAAACATATATACTTTGTGTGATTAGAACTATCATTGAAGTAAATAAAATGAAAT
CATTATAACAAAAAGGAATTTACATGGTTAGAAATTTTACTTTACCCTAGATACTTAATACTTATTAGCGTAGATTTTCCTTACATC
AACCTGTTTATTTCATTTTCTTTTCTTTTTAACGCAAATATATAGTTCCAATAGTATTTACCCTTGTTTTCTGTATGTAAAATTTTAG
GGATGAGTAGTGTGTATGCCTCATACCATATGTTCCATATATAATCTATGTTCGTGTCTACCAACCGTATAAGTAATCTAAAACAC
TTGAGATCAATTATATACACATTAATTGTCTAATGTTCAAGAATCAAGACCATATATTAATGACGATTATTGATGTGCTTATAGGTA
TGATGTTCATGTTGGGATACAAGACCCATACGAAGATTGTGTAGCGACGATGAGGCTTTACACGAGAATGAGATATCAGAAACA
CAAGATTGAAGCTTATCCTTTAGCTGCCGACGCGCACAACCGTAGCAATCAAGTGGCTTGGAGGCAGAACGAGTTCGAGAGGA
TGTCTCCTGATGAAATGCTTTCCATCTCTCGATCCGACTACTGTTGCTGGTGCTTGGACTCCCTCGCCTGA
SEQIDNO: 139
MDCRSMESSETLRNKCAACYKQFNKMEHLVEHMKISYHSGHEPTCGVCKKHCRSFESLREHLIGPLPKQECKNIFSILGCRFCLMILE
TPNARRIHQERCQFSSVNAGLTTRMAALGIRDKDMIDYTSSRSPKVVALSCKMVGGGSDGSLDLCARVCITDEGDNVVFHTYVKPSM
AVTNYRYEKTGIRPENLRDAMPLKHAQRKIQEFLCNGEPMWKIRPRGGKGRILVGHGLDHDLDRLQLEYPSSMIRDTAKYPPLMKTS
KLSNSLKYLTQAYLGYDVHVGIQDPYEDCVATMRLYTRMRYQKHKIEAYPLAADAHNRSNQVVWRQNEFERMSPDEMLSISRSDYY
CWCLDSLA
SEQIDNO: 140
ATAATACAAAAAAAATCATCAAAACAGTTGCTTAAACTCTCTCATATCTTAAGTTGCTACGTTTTTGTTTGTATTTTCCGATACGTA
CCATGGATTGCAGATCTATGGAGTCATCGGAAACCCTAAGGAACAAATGCGCAGCTTGTTATAAGCAATTCAACAAAATGGAAC
ATTTAGTGGAGCACATGAAGATCTCGTATCACTCCGGTCATGAGCCTACCTGCGGCGTTTGCAAGAAACATTGCCGATCTTTTG
AGTCCCTCCGAGAACACCTCATAGGGCCATTGCCAAAACAAGAATGCAAGAACATTTTCAGCATTCTCGGATGCAGATTCTGCT
TGATGATCCTCGAAACCCCGAACGCTCGTAGGATCCATCAAGAGAGATGCCAATTTTCGAGCGTCAATGCGGGATTGACGACT
CGTATGGCGGCCTTAGGCATAAGAGATAAGGACATGATCGACTACACGTCGTCTAGGTCCCCAAAAGTGGTTGCACTTTCTTGC
AAGATGGTAGGAGGGGGAAGCGACGGGTCGCTGGATCTATGTGCAAGGGTTTGCATAACGGATGAGGGCGACAATGTTGTGT
TCCATACGTACGTGAAACCGTCAATGGCCGTGACGAACTATAGGTATGAAAAGACCGGCATACGTCCGGAGAATCTGAGGGAC
GCAATGCCGTTGAAACATGCACAAAGAAAGATTCAAGAGTTTCTTTGTAATGGAGAACCCATGTGGAAGATTCGTCCAAGAGGT
GGGAAAGGGAGGATTCTCGTGGGACATGGCCTCGATCACGATCTTGACCGCCTTCAACTTGAATATCCTTCTTCCATGATAAGG
GATACTGCGAAATACCCTCCCTTGATGAAAACAAGCAAGCTGAGCAATTCCCTCAAGTACTTGACCCAAGCCTATCTCGGGTAT
GATGTTCATGTTGGGATACAAGACCCATACGAAGATTGTGTAGCGACGATGAGGCTTTACACGAGAATGAGATATCAGAAACAC
AAGATTGAAGCTTATCCTTTAGCTGCCGACGCGCACAACCGTAGCAATCAAGTGGTTTGGAGGCAGAACGAGTTCGAGAGGAT
GTCTCCTGATGAAATGCTTTCCATCTCTCGCTCCGACTACTATTGCTGGTGCTTGGACTCCCTCGCTTGATTTCTGAAACTCTGG
GGTTAAGTTGAGGAATTTCACATAATAACTTCGTAAACTTTTTGAAGATAATAAATTTAGTAGCGTAATGTCGAATAAGAATCAGT
TGTTATTTTTAATATGTACTAGTATATAAATTTTATAGTATGTGTCGTGTGAGACGCATATAAATTGTTA
SEQIDNO: 141
ATGGATTGCAGATCTATGGAGTCATCGGAAACCCTAAGGTAACCTATCCTCCATCGTACGTGTATTAGATTATATCAAGCGTTTA
TATGAAAATATCGAAAAGAACTCAGGGAGGGTGTCATCCACTATCACATCCAAATTCTATAATCAATTGATGAAAAACTAGAAATA
ATTTGTTTTCCTTAAATTATAGTTTTATACATATGTACACGTACGTGCAGGAACAAATGCGCAGCTTGTTATAAGCAATTCAACAA
AATGGAACATTTAGTGGAGCACATGAAGATCTCGTATCACTCCGGTCATGAGCCTACCTGCGGCGTTTGCAAGAAACATTGCCG
ATCTTTTGAGTCCCTCCGAGAACACCTCATAGGTAAATGATAACCTATATATATTAAGAGAAATGTATCGTTTTATACATTCACGA
CGCATATGTGTATTTATTTTTAATTGTGTTATATACATAGGGCCATTGCCAAAACAAGAATGCAAGAACATTTTCAGCATTCTCGG
ATGCAGATTCTGCTTGATGATCCTCGAAACCCCGAACGCTCGTAGGATCCATCAAGAGAGATGCCAATTTTCGAGCGTCAATGC
GGTAATCTTTTCTTCTCATATTTGCATAATTAAAATTGTTAATAATCAACCGCACAAAATATATTTATTCTTACATATATGATATGAT
TTTCTCTGTTTCAGGGATTGACGACTCGTATGGCGGCCTTAGGCATAAGAGATAAGGACATGATCGACTACACGTCGTCTAGGT
CCCCAAAAGTGGTTGCACTTTCTTGCAAGATGGTAGGAGGGGGAAGCGACGGGTCGCTGGATCTATGTGCAAGGGTTTGCATA
ACGGATGAGGGCGACAATGTTGTGTTCCATACGTACGTGAAACCGTCAATGGCCGTGACGAACTATAGGTATGAAAAGACCGG
CATACGTCCGGAGAATCTAAGGGACGCAATGCCCTTGAAACATGCACAAAGAAAGATTCAAGAGTTTCTTTGTAATGGAGAACC
CATGTGGAAGATTCGTCCAAGAGGTGGGAAAGGGAGGATTCTCGTGGGACATGGCCTCGATCACGATCTTGACCGCCTTCAAC
TTGAATATCCTTCTTCCATGATAAGGTGGCATTTCTATAAAGAAAAAATATAAAGAGGATATATATATAATAGAAAAAGACTAGCA
TAGCACCAAACCAAGTTTTTCCTCAAACTAGCACTCAAGGATCAAAGTCACAAAAATAGGTTTCATTAAAAAGATAAATATACTCT
TAGGGTTTAGAGTTAGGGGTGGAGTTTTTGAATTAGAGTTTAAAATTTTATAAAATAAAAAATAAATACTAAAAAATTAAAAATAAA
AATTAAAAAAACAGTTTCAAAAAGTATTTTTGAATTATAAAAAGAAAATTTAAAAAAAAATAAAAAAAATTTCGAAAAAAAATTATAA
AAAATGTCGAATCTGAAAACATATAATCTGAAACTATAAAAAAAAATTTCATTTTTTTTTATTTTTATTTTATTTGTTTTTATTTATTTT
GTTTGTTAATTTAATTTTAAACCAAAAGTATTAGACATATTTTATCATTTAATGAATGTCATTTTTGTGACTTTTTCCTTCTAATGTTA
TTTTTGAGATAAAAACTCAAAAGGTGCTATTATTGACAATTGTCACTAGATTCCTTTAGTGTTACTTGAAATCTTTCAATATAGCCA
ATGATTTTATGTGTGTTTTTTATATGACGGGATTTTGTATGATCTTATTGCTGTATGTAGGGATACTGCGAAATACCCTCCCTTGA
TGAAAACAAGCAAGCTGAGCAATTCCCTCAAGTACTTGACCCAAGCCTATCTCGGGTAATATATCTAATTTTGTTTTTGTAATAAT
ATTTTCAAACCGGGTGTTGATGCTTTGTATGGGCTTAAAGTTATAAGGGAGGGCTTCAAAACGTAAGATTCACATGTTTTCTAAT
ATATTCATTTCAAAATTTACACATAAACATATATATATAGTTTGTGTGACTAGAAATATCATTGAAGTAAATAAAATGAATTCATTAT
AATAAAAACAAATTTACATGGTTAGAAATTTTACTTTACCCTAGATACTTAATACTTATTAGCGTTGATTTTCCTTACATCAACCTG
GTTATTTCATTTTCTTTTCTTTTTAACGCAAATATATAGTTCCAATAGTATATACCCTTCTTTTCTGTATGTAATATTTTAAGATTTAT
TCCTAGGTTCACCCTTTAGGTTTACCAACTAATAAGATTTTGTTATTTCATATTCGATATCTTTCAAAAAAATGAGACAAAATATTA
TCAAATTATATTATGTTTTTAAAATAAAAAGTAAAAAAAAAAAAATAATAGTAGTTACAACAAAATAATTTAAAAAAATATTTTTAAC
GTCGTCAGTAAAACACTAAACCCTAAATGCTAAACCATAAACCATTGGATAACCCCTAAACCATTAGATAAATCCTAAACTCTAAA
TCAAAAACACTAAACACTAAACCCTAAATCCTAAATCCTTGAGTGTTTTAATGTTTAGTGTTTTGATTTAGAAATTAATATTTATCC
AAGGGCTTAGAGTTTACCCAAATGTTTAGGATTTATATATGGATTAAGATTTAGAATTTAATGTTTTGCTGACGACGTTAAATATAT
ATATATATATATATTTTTTTTTTTTGTAACTACTACTATTTTAAATTTATTTATTTATTAATTTTTATTTTTAAAACATAATATAATTTGA
CAATATTTTGTTTCATTTAAAAAAAATATCAAATATAAAATAACACAATTTTATTGGTTGATAAAGCTAGAGGTTTACTCTAGGGGG
TGAACCCAAAAATAAGTCATATTTTAGGGATGAGTAGTGTGTATGCCTCATACCATATGTTCAATATATAATATATGTTCGTGTCC
ATCAACCGTATAAGTAATCTAAAACACTTGAGATCAATTATATACACATTAATTGTCTAATGTTCAAGAATCAAGACCATATATTAA
TGATGATTATTGATGTGCCTATAGGTATGATGTTCATGTTGGGATACAAGACCCATACGAAGATTGTGTAGCGACGATGAGGCTT
TACACGAGAATGAGATATCAGAAACACAAGATTGAAGCTTATCCTTTAGCTGCCGACGCGCACAACCGTAGCAATCAAGTGGTT
TGGAGGCAGAACGAGTTCGAGAGGATGTCTCCTGATGAAATGCTTTCCATCTCTCGCTCCGACTACTATTGCTGGTGCTTGGAC
TCCCTCGCTTGA
SEQIDNO: 142
MDYRSSIESSETLRNKCAACYRQFNKMEHLVVHMKISYHSGHEPTCGVCKKHCRSFESLREHLIGPLPKQECKNIFSLRGCRFCMMI
LESPNARRTHQERCQFSSANAGLTTRMAALGLRDKAMIDYTSSRSPKVVALSCKMVGGGSDGSLDLCARVCITDESDNVVFHTYVK
PSMIVTNYRYGTTGIRPENLRDAMPLKHAQRKIQEFLCNGEPMWKIRPRGGRGRILVGHGLDHDLDRLQLEYPSSMMRDTAKYPPL
MKTSKLSNSLKYLTQAYLGYDIHVGIQDPYEDCVATMRLYTRMRYQKHKTEAYPQAADAQNRSNQVAWRQNEVERMSPDEMLSISR
SDYYCWCLDSLA
SEQIDNO: 143
ATGGACTACAGATCATCTATAGAATCATCAGAAACCCTAAGGAACAAGTGCGCAGCTTGTTATAGGCAATTCAACAAAATGGAAC
ATTTAGTGGTGCACATGAAGATCTCTTATCACTCCGGTCATGAGCCTACTTGTGGCGTTTGCAAGAAACATTGCCGATCTTTTGA
GTCCCTCCGGGAACATCTCATAGGACCATTGCCAAAACAAGAATGCAAGAACATTTTTAGCCTTCGCGGATGCAGATTCTGCAT
GATGATCCTGGAAAGCCCGAACGCTCGTAGGACCCATCAAGAGAGATGTCAATTTTCGAGCGCCAATGCTGGATTGACGACTC
GTATGGCGGCCTTAGGCCTAAGAGATAAGGCCATGATCGACTACACGTCCTCGCGGTCCCCAAAAGTGGTTGCACTCTCTTGC
AAGATGGTAGGAGGAGGAAGCGACGGGTCGTTGGATCTATGCGCAAGGGTCTGCATAACGGATGAGAGTGACAACGTTGTGT
TCCACACGTACGTGAAACCGTCAATGATCGTGACGAACTATAGGTACGGGACGACCGGGATACGTCCGGAGAATCTAAGGGAC
GCCATGCCGTTGAAACATGCTCAAAGAAAGATCCAAGAATTTCTTTGTAATGGAGAACCTATGTGGAAGATTCGTCCAAGAGGT
GGGAGAGGGAGGATTCTCGTGGGACATGGGCTCGACCACGATCTTGACCGCCTTCAACTTGAATATCCTTCTTCCATGATGAG
GGATACTGCAAAATACCCTCCGTTGATGAAAACAAGCAAGCTGAGCAATTCTCTCAAGTACTTAACCCAAGCCTATCTCGGGTAT
GATATTCATGTTGGGATACAAGACCCATACGAAGATTGTGTAGCGACGATGAGGCTGTACACAAGAATGAGATATCAGAAACAC
AAGACTGAAGCTTATCCTCAAGCTGCCGACGCACAGAACCGTAGCAATCAAGTGGCTTGGCGGCAGAACGAGGTCGAGAGGA
TGTCTCCTGATGAAATGCTCTCCATCTCTCGCTCCGACTACTATTGCTGGTGCTTGGACTCCCTCGCTTGA
SEQIDNO: 144
ATGGACTACAGATCATCTATAGAATCATCAGAAACCCTAAGGTAGCCATTTTTTCATCCTACGCGTGTTGTACCATGGATCATCG
TTTAAAATATTAAAAAAGAAGACTCATGGAGCGGCATGCAATATCATACCCAAATTCTATATGTGATAAAATGATTAAAACTAAAA
AAAAAGGTAAATATATTTATATATGTACACGTGCAGGAACAAGTGCGCAGCTTGTTATAGGCAATTCAACAAAATGGAACATTTA
GTGGTGCACATGAAGATCTCTTATCACTCCGGTCATGAGCCTACTTGTGGCGTTTGCAAGAAACATTGCCGATCTTTTGAGTCC
CTCCGGGAACATCTCATAGGTAAACACGAATATCTGTATATATATATAAAGATAAATATATCGTTTTTATATACACTATGAATTAGT
ATTTTTATCTTTAATTCTGTAATTAATGTCAATAGGACCATTGCCAAAACAAGAATGCAAGAACATTTTTAGCCTTCGOGGATGCA
GATTCTGCATGATGATCCTGGAAAGCCCGAACGCTCGTAGGACCCATCAAGAGAGATGTCAATTTTCGAGCGCCAATGCTGTAA
TCTTCGTAGTTCTTATCTCAACATACTTCACTTCTCATATTTGCGTATTTTAACTTGTAAAGAATCAACCACATGAAAGATATGTTC
TTACATATATATCATATGATTTCTTCCTTTCAGGGATTGACGACTCGTATGGCGGCCTTAGGCCTAAGAGATAAGGCCATGATCG
ACTACACGTCCTCGCGGTCCCCAAAAGTGGTTGCACTCTCTTGCAAGATGGTAGGAGGAGGAAGCGACGGGTCGTTGGATCTA
TGCGCAAGGGTCTGCATAACGGATGAGAGTGACAACGTTGTGTTCCACACGTACGTGAAACCGTCAATGATCGTGACGAACTA
TAGGTACGGGACGACCGGGATACGTCCGGAGAATCTAAGGGACGCCATGCCGTTGAAACATGCTCAAAGAAAGATCCAAGAAT
TTCTTTGTAATGGAGAACCTATGTGGAAGATTCGTCCAAGAGGTGGGAGAGGGAGGATTCTCGTGGGACATGGGCTCGACCAC
GATCTTGACCGCCTTCAACTTGAATATCCTTCTTCCATGATGAGGTAACATTTCTATAAGAAAAAGATAAAGAGGATACATACTTA
TAACTATATTTCTTTCAATATAACCTATATTTTTATTTTTTTTTTTTTTTTTTTTTTTTTTTTTGGTCAACTAAATAAATGAATGCAGTT
AAGGGTAATTGAACCCAGCATTTCTAGCACTGGTAATTTCTCTTAGAACCACTAGGCTAAAGTCACTTTTTTCCTATATTTTTATG
TTAGCTTGTTTTTTTATCGTGGAATTTCTATATCATTTTGTATGTATATTGGTGTATGTAGGGATACTGCAAAATACCCTCCGTTGA
TGAAAACAAGCAAGCTGAGCAATTCTCTCAAGTACTTAACCCAAGCCTATCTCGGGTAATATATCTAGTTTTGTTTTTGTAATATT
ACTTTACTTTTGAGTCTAACATATTGGAAAGTGAATATAGATGCATGCATACGTTATGTATATTGATACATATGTGAAGCAAATATT
ATCAATTAATTATTTCTCTTTTTTTTGCTAACTAATTAATTCTTTCTCAAAAAAATATCATCAATTAATTTATTAGGTGTTAACTATTC
ATGCTTTGTATGGCTTAAAGTTATAAGGAAGAGAGGATTCAAAACGTGAAAGATTAACATGTTTTAAAATGTATTCATCGACATCT
ACATATATATTTTATATATATACCATATTATATATAGATTGTATGGCTAAACTATCCAACAAAAAATGAAACACTTTACATGGTTAAT
TAGACATTTTTATTTTACACGAGATGCTATTACCGTAAATTTTTCTCTCCAAATCGAATACATCAACCTGCTTATTTCGTTTTTTTTT
TTGCCAATCGCAAATTATTCAAATAGTACAACAGAGTTTTTTTTTTGTATTTTAAATACTATGGATGAGTGTGTATGCCTCCTCATA
TCATATGTTGCATATAATATATGTTCGTGTCTACGCACCGAATAAGTAATATAAAACATTTGAGATCAATTAGACCCATTATCTGAT
GTTCAAGATTATTAACTAATGATGATTTTTGATGGTAATTATTTAGGAAACTTAACATTTACACCCAAAAAAAACTAATGATGATTA
TTGATGTGCTTATAGGTATGATATTCATGTTGGGATACAAGACCCATACGAAGATTGTGTAGCGACGATGAGGCTGTACACAAG
AATGAGATATCAGAAACACAAGACTGAAGCTTATCCTCAAGCTGCCGACGCACAGAACCGTAGCAATCAAGTGGCTTGGCGGC
AGAACGAGGTCGAGAGGATGTCTCCTGATGAAATGCTCTCCATCTCTCGCTCCGACTACTATTGCTGGTGCTTGGACTCCCTCG
CTTGA
SEQIDNO: 145
MDYRSPMESSETLRNKCAACYRQFNKMEHLVEHMKISYHSGHEPTCGVCKKHCRSFESLREHLIGPLPKQECKNIFSLRGCRFCMM
ILESPNARRIHQERCQFSSVNAGLTTRMAALGLRDKVMIDYTSSRSPKMVALSCKMVGGGSDGSLDVCARVCITDENDNVVFHTYVK
PSMVVTNYRYETTGIRPENLRDAMPLKHAQRKIQEFLCNGEPMWKIRPRGGKGRILVGHGLDHDPDRLQLEYPSSMMRDTAKYPPL
MKTSKLSNSLKYLTQAYLGYDIHVGIQDPYEDCVATMRLYTRMRYQKHKIEAYPLPADVQNRSNQVAWRQSEVERMSPNEMLSISRS
DYYCWCLDSLA
SEQIDNO: 146
ATGGATTACAGATCACCTATGGAGTCATCGGAAACCCTAAGGAACAAGTGCGCAGCTTGTTATAGGCAATTCAACAAAATGGAA
CATTTGGTGGAACACATGAAGATCTCTTATCACTCCGGTCATGAGCCTACTTGTGGAGTTTGCAAGAAACATTGCCGATCTTTTG
AGTCCCTCCGCGAACATCTCATAGGACCATTGCCAAAACAAGAATGCAAGAATATTTTTAGCCTTCGCGGATGCAGATTCTGCA
TGATGATCCTCGAAAGCCCTAACGCTCGTAGGATTCATCAAGAGAGATGTCAATTTTCGAGCGTCAATGCTGGATTGACGACTC
GTATGGCAGCCTTAGGCCTAAGAGATAAAGTCATGATCGACTACACGTCATCACGGTCTCCAAAAATGGTTGCACTCTCTTGCA
AGATGGTAGGAGGAGGAAGCGACGGGTCGTTGGATGTATGCGCAAGGGTTTGCATAACGGATGAGAACGACAACGTTGTGTT
CCATACGTACGTGAAACCGTCAATGGTCGTGACGAACTATAGGTACGAGACGACTGGTATACGTCCAGAGAATCTGAGGGACG
CAATGCCGTTGAAACATGCACAAAGAAAGATTCAAGAATTTCTTTGTAATGGAGAACCCATGTGGAAGATTCGTCCAAGAGGTG
GGAAAGGGAGGATTCTCGTGGGACATGGGCTGGATCACGATCCTGACCGCCTTCAACTTGAATATCCTTCTTCCATGATGAGG
GATACTGCAAAATATCCTCCGTTGATGAAAACAAGCAAGCTGAGCAATTCTCTCAAGTACTTAACCCAAGCCTATCTCGGGTATG
ATATTCATGTTGGGATACAAGACCCATACGAAGATTGTGTAGCGACGATGAGGCTTTACACGAGAATGAGATATCAGAAACATA
AGATTGAAGCATATCCTTTACCAGCCGACGTGCAGAACCGTAGCAATCAAGTGGCTTGGAGGCAGAGCGAGGTCGAAAGGATG
TCTCCTAATGAAATGCTCTCCATCTCTCGCTCCGACTACTATTGCTGGTGCTTGGACTCCCTCGCTTGATTCCTAAAACTATGGG
GTTAATTTGGGAATCTCTCATAATTACTTCATAAACTTTTTCGAGATTTTAATTTAGTAGCGTAAAGTCGAATAAGCATTAATTCTG
ATTTTCTAATAGTATCTAAATTAATTTTATAGTATGTGTCGTGTGAGATGCGAATGTTGTTCCTGTACTTCTAATAACGCATATTGC
TCTTAATTATTAATAATTATAATTTTTGTTGAAATTAATTATCAAATCACATGTTAGCTTTTGTCTATAAATATATCTATATTTGTCTT
AG
SEQIDNO: 147
ATGGATTACAGATCACCTATGGAGTCATCGGAAACCCTAAGGTAACCATTTTCATCCTAAGCGTGTTATATCAAGCGTTTTAATT
GGAAAATAAATCCCAAAGAAAAACTCAATGGAGTGGCATGCAATATCATACCCAAATTCTGTAGTCAAGTGATGAAAAACTAAAA
TTTAAAGTTTTTGCTAAAATATATTTGTATATATATGTAAAAAAATATTTATGTATATATGTACACGTGCAGGAACAAGTGCGCAGC
TTGTTATAGGCAATTCAACAAAATGGAACATTTGGTGGAACACATGAAGATCTCTTATCACTCCGGTCATGAGCCTACTTGTGGA
GTTTGCAAGAAACATTGCCGATCTTTTGAGTCCCTCCGCGAACATCTCATAGGTAAACAAGAAGCTCTATATAAAGAGTGACATA
TAGTTTTATATATACGCTATGCATTTGTATGTTAATTTTTAATTGTGTAATGTCAATAGGACCATTGCCAAAACAAGAATGCAAGAA
TATTTTTAGCCTTCGCGGATGCAGATTCTGCATGATGATCCTCGAAAGCCCTAACGCTCGTAGGATTCATCAAGAGAGATGTCA
ATTTTCGAGCGTCAATGCTGTACGTAATCGTATTAATGTCCTTATCTCAATACTCCTCTTCTCATATTTGCGTAACTAAACTTGTAA
ATTGACCGCATGAAAAAAAAATTCTTATATAAATAATATAATATCATATGATTTGTCTTTTTTCAGGGATTGACGACTCGTATGGCA
GCCTTAGGCCTAAGAGATAAAGTCATGATCGACTACACGTCATCACGGTCTCCAAAAATGGTTGCACTCTCTTGCAAGATGGTA
GGAGGAGGAAGCGACGGGTCGTTGGATGTATGCGCAAGGGTTTGCATAACGGATGAGAACGACAACGTTGTGTTCCATACGTA
CGTGAAACCGTCAATGGTCGTGACGAACTATAGGTACGAGACGACTGGTATACGTCCAGAGAATCTGAGGGACGCAATGCCGT
TGAAACATGCACAAAGAAAGATTCAAGAATTTCTTTGTAATGGAGAACCCATGTGGAAGATTCGTCCAAGAGGTGGGAAAGGGA
GGATTCTCGTGGGACATGGGCTGGATCACGATCCTGACCGCCTTCAACTTGAATATCCTTCTTCCATGATGAGGTGACATTTCT
ATAAAGAAGAAAAGTAAAGAGGACACATATATATAACTAGATTCCTTATAGTGTTACTTGGTTTCTGGCAACATAACCCAGGTTTA
TGTGAGTTGTTTTATAATTAACGTGAATTTCTAATATCATTTTTATATATTGGTGTATGTAGGGATACTGCAAAATATCCTCCGTTG
ATGAAAACAAGCAAGCTGAGCAATTCTCTCAAGTACTTAACCCAAGCCTATCTCGGGTAATATATCTACTTTTGTTTCTGTAATAC
TACTTTCGAATATAACATTAATTTGGAAAATGAATATAGATATATGCATGCTTTACGTGTATTGATACATAAGTGTATCATGTACTA
TCCATTAATTTTTTGGTGATGTATGGGTATTGATGCTTTGTATGGGCTTAAAGCTAAAAGGAAGGGATTCAAAACGTGAAAAGAT
TCATATGTTATATAATATATACCATTTTCTAACAAAAACAAAAAGAACTTACATAGTTGGAGATTTTTAATTTACATGAGATGCTTA
CTAGCGTAATTTTTATCGCCAAGTTGACTACATCAACCTGTTTATTTCATTTGATTTCCTATCAATTACAAACAGTTCAACTAGTAT
ACCATTGTTTTGTATGTAAAACTTTAGGGATGAGTGTGTATGCCTAATAATATCATATGTTCCATATAATCTGTGTTCGTTTCTACA
CACCGTAGAAGTAATCTCAAACACTTGAGATCAATTAATTATACCCATTATCTAATGTACAACAATATATATTAAGGATGGTATTG
ATGTGCTTATAGGTATGATATTCATGTTGGGATACAAGACCCATACGAAGATTGTGTAGCGACGATGAGGCTTTACACGAGAAT
GAGATATCAGAAACATAAGATTGAAGCATATCCTTTACCAGCCGACGTGCAGAACCGTAGCAATCAAGTGGCTTGGAGGCAGA
GCGAGGTCGAAAGGATGTCTCCTAATGAAATGCTCTCCATCTCTCGCTCCGACTACTATTGCTGGTGCTTGGACTCCCTCGCTT
GATTCCTAAAACTATGGGGTTAATTTGGGAATCTCTCATAATTACTTCATAAACTTTTTCGAGATTTTAATTTAGTAGCGTAAAGTC
GAATAAGCATTAATTCTGATTTTCTAATAGTATCTAAATTAATTTTATAGTATGTGTCGTGTGAGATGCGAATGTTGTTCCTGTACT
TCTAATAACGCATATTGCTCTTAATTATTAATAATTATAGTTTTTGCTGAAATCTATACTATTATTTGGTAAGTAATTTTTCGCATTC
GAGCTATCACGTTAAAAGTTGGAGTAGTTAAAGTCATTGTTACCCTTAATGAATGATTAAATTTATTTTTAGATTATATTATTGATT
ATAAATTAATATTATAAAAAAATAGCCATATATAAAAACGAATTTTAAATTTATTAAATCAGATAATTTATTACAATTGATACAATAAA
AGTTAACTAAACATTAAATGTACTTTAAAAATAAAAATATAATTCTTATATATTTTGTGTTGTTATCCGAAGATAATATATTTTAATAT
ATTTTAAAAATAGATTTAAAAATTCAAAGAGAATTTTAATTTTATTAAATCAGATAATTCATTAAAATTGATATAATAAAAGTTATCTA
AACATTAAATTTATTTTAAAAATAAAAATATAAGTCTTTTATATTATTCTATTTTTTTTGTCATATCTTATATATTTTTTGTTATCCGAA
GATAATATATTTTAATATATTTTAAAAATAGATTGAAAAATTAAAATATTTAAGTTTTAAAATATATTATGTTATCCAAAAAATATTTC
ACATTATAATATTTTTAAAATAAATATAAATCTCTGTATATATATTTTTATGTATATATGAAGGTTTTCAAGTTTGTTTTAATAAAAGA
TATTTTATTAAAGTAAACAAAATATGGTATATAAAATTTTTATTATTTAATTAAATATTAAATATTTCAAAAAGCAAGAAAATAATTGA
TTTAATGGTTTTTAAATTGATAATATATTTAGTTGCAATTTTTTTGTAAAATTATTAAGCCCGCCAGTGCGGGCAACACACCTAGTT
AATTAGTAATCACTTGTTAGCTTTTGTCTATAAATATATCTATATTTTTCTTAGAGTTTTTAACACATATGAAAAAATAATGGAAATT
TTCTTTTTTTTTTATAACAAATTTACACAATAATGAGAGGTGTTTCAAAAAAAAAACACAATAAGGAGAGATTTTTGTTGAAATTAAT
TATCAAATCACATGTTAGCTTTTGTCTATAAATATATCTATATTTGTCTTAG
SEQIDNO: 148
MDYRSSIESSETLRNKCAACYRQFNKMEHLVEHMKISYHSGHEPTCGVCKKHCRSFESLREHLIGPLPKQECKNIFSLRGCRFCMMI
LESPNARRIHQERCHFSSLNAGLTTRMAALGLRDKALIDYTSSRFPKVVALSCKMVGGGSDGSLDLCARVCITDESDNVVFHTYVKPS
MVVTNYRYGTTGIRPENLREAMPLKHAQRKIQEFLCNGEPMWKIRPRGGRGRILVGHGLDHDLDRLQLEYPSSMMRDTAKYPPLMK
TSKLSNSLKYLTQAYLGYDIHVGIQDPYEDCVATMRLYTRMRYQKHKIEAYPQAADSQNRSNQVAWRQNEVEKMSPDEMLSISRSD
YYCWCLDSLA
SEQIDNO: 149
ATGGATTACAGATCATCTATCGAATCATCAGAAACCCTAAGGAACAAGTGCGCAGCTTGTTATAGGCAATTCAACAAAATGGAAC
ATTTAGTGGAGCACATGAAGATCTCTTATCACTCCGGTCATGAGCCTACTTGTGGCGTTTGCAAGAAACATTGCCGATCTTTTGA
GTCCCTCCGGGAACATCTCATAGGACCATTGCCAAAACAAGAATGCAAGAACATTTTTAGCCTTCGCGGATGCAGATTCTGCAT
GATGATCCTCGAAAGCCCAAACGCTCGTAGGATCCATCAAGAGAGATGTCATTTTTCGAGCCTCAATGCTGGATTGACGACTCG
TATGGCGGCCTTAGGCCTAAGAGATAAGGCCTTAATCGACTACACGTCCTCGCGGTTTCCAAAAGTGGTGGCACTCTCTTGCAA
GATGGTAGGAGGAGGAAGCGATGGGTCATTGGATCTATGCGCAAGGGTCTGCATAACTGATGAGAGTGACAACGTTGTGTTTC
ACACGTACGTGAAACCGTCAATGGTCGTGACGAACTATAGGTACGGGACGACTGGGATACGTCCGGAGAATTTAAGGGAAGCA
ATGCCGTTGAAACATGCTCAAAGAAAGATCCAAGAATTTCTTTGTAATGGAGAACCCATGTGGAAGATTCGTCCAAGAGGTGGG
AGAGGGAGGATTCTCGTGGGACATGGGCTCGACCACGATCTTGACCGCCTTCAACTTGAATATCCTTCTTCCATGATGAGGGAT
ACTGCAAAATACCCTCCGTTGATGAAAACAAGCAAGCTGAGCAATTCTCTCAAGTACTTAACCCAAGCCTATCTCGGGTATGATA
TTCATGTTGGGATACAAGACCCATACGAAGATTGTGTAGCGACGATGAGGCTGTACACAAGAATGAGATATCAGAAACACAAGA
TTGAAGCTTATCCTCAAGCTGCCGACTCACAGAACCGTAGCAATCAAGTGGCTTGGCGGCAGAACGAGGTCGAGAAGATGTCT
CCTGATGAAATGCTCTCTATCTCTCGCTCCGACTATTATTGCTGGTGCTTGGACTCCCTCGCTTGATTTCTAAAACTATGGGGCT
AATTTGAGTAATCTCTCACAACTACTACACAAACTCTTTTCGGGATTCTAATCTAGTA
SEQIDNO: 150
ATGGATTACAGATCATCTATCGAATCATCAGAAACCCTAAGGTAACCATTTTTTTCATCATACTCGTGTTATAACTTATATTAAGC
GTTCTAATAAAAATATTCTAAAGGAAGACTCATGGAGTGGCATGCAATATCATACCCAAACCCAAATTCTATATGTGATAAACTGA
CGAAAACTAAAAAAAATAGGTAGACATATTTATATATGTACACGTGCAGGAACAAGTGCGCAGCTTGTTATAGGCAATTCAACAA
AATGGAACATTTAGTGGAGCACATGAAGATCTCTTATCACTCCGGTCATGAGCCTACTTGTGGCGTTTGCAAGAAACATTGCCG
ATCTTTTGAGTCCCTCCGGGAACATCTCATAGGTAAATAAGAATATCTGTATATATATATATATATACATACATATATAAAGATAAA
TATATCGTTTTTATATACACTATGCAATAGCATTTTTTATCTTTAATTTTGTAATGTCAATAGGACCATTGCCAAAACAAGAATGCA
AGAACATTTTTAGCCTTCGCGGATGCAGATTCTGCATGATGATCCTCGAAAGCCCAAACGCTCGTAGGATCCATCAAGAGAGAT
GTCATTTTTCGAGCCTCAATGCTGTAATCTTCTTAGTTCATATCTCAATATACTTCACTTCTCATTTTTGCTTATTCTAACTTATAAA
GAATCAGCCGCATGAAAGATATGTTCTTACATTTATATCATATAATTTCTTCATTTCAGGGATTGACGACTCGTATGGCGGCCTTA
GGCCTAAGAGATAAGGCCTTAATCGACTACACGTCCTCGCGGTTTCCAAAAGTGGTGGCACTCTCTTGCAAGATGGTAGGAGG
AGGAAGCGATGGGTCATTGGATCTATGCGCAAGGGTCTGCATAACTGATGAGAGTGACAACGTTGTGTTTCACACGTACGTGA
AACCGTCAATGGTCGTGACGAACTATAGGTACGGGACGACTGGGATACGTCCGGAGAATTTAAGGGAAGCAATGCCGTTGAAA
CATGCTCAAAGAAAGATCCAAGAATTTCTTTGTAATGGAGAACCCATGTGGAAGATTCGTCCAAGAGGTGGGAGAGGGAGGATT
CTCGTGGGACATGGGCTCGACCACGATCTTGACCGCCTTCAACTTGAATATCCTTCTTCCATGATGAGGTAACATTTCTATAAGA
AACAGATAAAGAGAAAGAGGATACGTACTTATAACTATATTTCTTTCAATATAACCTATATTTTATGTTAGCTTGTTATATATCGTA
GAATTTCTATATCATTTTGTATGTATACTGGTGTACGTAGGGATACTGCAAAATACCCTCCGTTGATGAAAACAAGCAAGCTGAG
CAATTCTCTCAAGTACTTAACCCAAGCCTATCTCGGGTAATATATCTAGTTTTGTTTCTGTAATATTACTTTTGAGTCTAACATATT
GGAAAGTGAATATAGATGCATGCATACGTTATGTATATTGAAACATATATATGTGAAGCAAATATTATCAATTAATTCTTTCTCTCT
TTTTTTTTGGTAACTATTTAATTCTTTCTCAAAAAAAAATATTATCAATTAATTTATTTGGTGTTATGCTATTGATGCTTTGTATACGC
TTAAAGTTATAAAGAATAGAGGATTCAAAACGTGAAAGATTCACATGTTTTAAAATATAGTCATCGACATCTACATATATATATTAT
ATATATACCATATTATATATAGATTGTATGGCTAAACTATCCAACAAAAAAACGAAACACTTATTTCGTTTTCTTTTTTTTTTTGCCA
ATCGCAAATTATTCAAATAGTACAACAGATTTTTTTTTGTATTTTAAATACTATGTATGAGTGTGTATGCCTCATATCGTATGTTGC
ATATAATATATGTTCGTGTCTACGCATCGAATAAGTAATATAAAACATTTGAGATCAATTAGACACATTATCTGATGTTCAAGATTA
TTAACTAATGATGATTATTGATGTGCTTATAGGTATGATATTCATGTTGGGATACAAGACCCATACGAAGATTGTGTAGCGACGA
TGAGGCTGTACACAAGAATGAGATATCAGAAACACAAGATTGAAGCTTATCCTCAAGCTGCCGACTCACAGAACCGTAGCAATC
AAGTGGCTTGGCGGCAGAACGAGGTCGAGAAGATGTCTCCTGATGAAATGCTCTCTATCTCTCGCTCCGACTATTATTGCTGGT
GCTTGGACTCCCTCGCTTGA
SEQIDNO: 151
MDCRSSMESSETLRNKCAACYKQFNKFEHLVEHMKISYHSGHEPMCGVCKKHCRSFESLREHLIGPLPKQECKNIFSFRGCRFCLMI
LETPNARRIHQERCQFSSVNAGLTTRMAALGVRDKAMIDYTSSRSPKVVALSCKMVGGGSDGSLDLCARVCITDESDNVVFHTYVKP
PMPVTNYRYEKTGIRPENLRDAMPLKHAQRKIQEFLCNGEPMWKIRPRGGKGRILVGHGLDHDLDRLQLEYPSSMIRDTAKYPPLMK
TSKLSNSLKYLTQAYLGYDVHVGIQDPYEDCVATMRLYTRMRYQKHKIEAYPLAADAHNRSNQVAWRQNEFERMSPDEMLSISRSD
YYCWCLDSLA
SEQIDNO: 152
ATGGATTGCAGATCATCTATGGAGTCATCGGAAACCCTAAGGAACAAGTGCGCAGCTTGTTATAAACAATTCAACAAATTCGAAC
ATTTAGTGGAGCACATGAAGATCTCGTATCACTCCGGTCATGAGCCTATGTGTGGCGTTTGCAAGAAACATTGCCGTTCTTTTGA
GTCCCTCCGGGAACATCTCATAGGGCCATTGCCAAAACAAGAATGCAAGAACATTTTCAGCTTTCGCGGATGCAGATTCTGCTT
GATGATCCTCGAAACCCCGAACGCTCGTAGGATCCATCAAGAGAGATGCCAATTTTCGAGCGTCAATGCTGGATTGACGACTC
GTATGGCGGCCTTAGGCGTAAGAGATAAGGCCATGATCGACTACACGTCGTCTAGATCCCCAAAAGTGGTTGCACTCTCTTGC
AAGATGGTAGGAGGGGGAAGCGACGGGTCGCTGGATCTATGCGCAAGGGTTTGCATAACGGATGAGAGCGACAACGTTGTGT
TCCATACGTACGTGAAACCGCCAATGCCCGTGACGAACTATAGGTATGAGAAGACCGGCATACGTCCGGAGAATCTAAGGGAC
GCAATGCCCTTGAAACATGCACAAAGAAAGATTCAAGAGTTTCTTTGTAATGGAGAACCCATGTGGAAGATTCGTCCAAGAGGT
GGGAAAGGGAGGATTCTCGTGGGACATGGCCTCGATCACGATCTTGACCGCCTTCAACTTGAATATCCTTCTTCCATGATAAGG
GATACTGCGAAATACCCTCCGTTGATGAAAACAAGCAAGCTGAGCAATTCCCTCAAGTACTTGACCCAAGCCTATCTCGGGTAT
GATGTTCATGTTGGGATACAAGACCCATACGAAGATTGTGTAGCGACGATGAGGCTTTACACGAGAATGAGATATCAGAAACAC
AAGATTGAAGCTTATCCTTTAGCTGCCGACGCGCACAACCGTAGCAATCAAGTGGCTTGGAGGCAGAACGAGTTCGAGAGGAT
GTCTCCTGATGAAATGCTTTCCATCTCTCGATCCGACTACTATTGCTGGTGCTTGGACTCCCTCGCCTGA
SEQIDNO: 153
ATGGATTGCAGATCATCTATGGAGTCATCGGAAACCCTAAGGTAACCTATCTTCCATCGTACGTGTATTATATTATATCAAGCGT
TTATATGAAAATATCGAAAAGAACTCATGGAGGATGTCATCCAGTATCACACCCAAATTCTATAATCAATTGATGAAAAATAAGAA
ATAAAGTTTTTCTTAATTTATAGTTTTTATACATATGTACACGCACGTGCAGGAACAAGTGCGCAGCTTGTTATAAACAATTCAAC
AAATTCGAACATTTAGTGGAGCACATGAAGATCTCGTATCACTCCGGTCATGAGCCTATGTGTGGCGTTTGCAAGAAACATTGC
CGTTCTTTTGAGTCCCTCCGGGAACATCTCATAGGTAAATGATAAGCTATATATATTAAGAGAAATATATCGTTTTATATATTCAC
GACGCATATGTGTATTTATTTTTTATTGTGCTATATTATTAGGGCCATTGCCAAAACAAGAATGCAAGAACATTTTCAGCTTTCGC
GGATGCAGATTCTGCTTGATGATCCTCGAAACCCCGAACGCTCGTAGGATCCATCAAGAGAGATGCCAATTTTCGAGCGTCAAT
GCTGTAATCTTTTCTTCTAATATTTGCGCAAATTAAAATTGTTAATAATCAACCGCATAACAATATATTCTTACATATTTTCTATGTA
TCAGGGATTGACGACTCGTATGGCGGCCTTAGGCGTAAGAGATAAGGCCATGATCGACTACACGTCGTCTAGATCCCCAAAAG
TGGTTGCACTCTCTTGCAAGATGGTAGGAGGGGGAAGCGACGGGTCGCTGGATCTATGCGCAAGGGTTTGCATAACGGATGA
GAGCGACAACGTTGTGTTCCATACGTACGTGAAACCGCCAATGCCCGTGACGAACTATAGGTATGAGAAGACCGGCATACGTC
CGGAGAATCTAAGGGACGCAATGCCCTTGAAACATGCACAAAGAAAGATTCAAGAGTTTCTTTGTAATGGAGAACCCATGTGGA
AGATTCGTCCAAGAGGTGGGAAAGGGAGGATTCTCGTGGGACATGGCCTCGATCACGATCTTGACCGCCTTCAACTTGAATAT
CCTTCTTCCATGATAAGGTGGCATTTCTATAAAGAAAAACAAAATATAAAGAGGATATATACGTATATAACTAGATTCCTTTAGTG
TTACTTGAAATCTTTCAATATAGCCCATGATTTTATGTGTGTTTTTTATATGACGGGATTTTGTATCATTTTATTGCTGTATGTAGG
GATACTGCGAAATACCCTCCGTTGATGAAAACAAGCAAGCTGAGCAATTCCCTCAAGTACTTGACCCAAGCCTATCTCGGGTAA
TATATCTAATTTTGTTTTTGTAATAATATTTTCGAACCTAACACATTAATTTGGAAAATGATTATAGATGCATGCATACATTACCTAT
ATTGATACACATATATATCATAATAGGTGGGTATTGATGTTTGTATGGGCTTAAATGGGAGGGCTTCAAAACGTAAGATTTACAT
GTTTTCAAATATATTCATTTCAAAATTTACACATAAACATATATACTTTGTGTGATTAGAACTATCATTGAAGTAAATAAAATGAAAT
CATTATAACAAAAAGGAATTTACATGGTTAGAAATTTTACTTTACCCTAGATACTTAATACTTATTAGCGTAGATTTTCCTTACATC
AACCTGTTTATTTCATTTTCTTTTCTTTTTAACGCAAATATATAGTTCCAATAGTATTTACCCTTGTTTTCTGTATGTAAAATTTTAG
GGATGAGTAGTGTGTATGCCTCATACCATATGTTCCATATATAATCTATGTTCGTGTCTACCAACCGTATAAGTAATCTAAAACAC
TTGAGATCAATTATATACACATTAATTGTCTAATGTTCAAGAATCAAGACCATATATTAATGACGATTATTGATGTGCTTATAGGTA
TGATGTTCATGTTGGGATACAAGACCCATACGAAGATTGTGTAGCGACGATGAGGCTTTACACGAGAATGAGATATCAGAAACA
CAAGATTGAAGCTTATCCTTTAGCTGCCGACGCGCACAACCGTAGCAATCAAGTGGCTTGGAGGCAGAACGAGTTCGAGAGGA
TGTCTCCTGATGAAATGCTTTCCATCTCTCGATCCGACTACTATTGCTGGTGCTTGGACTCCCTCGCCTGA
SEQIDNO: 154
MESSETLRNKCAACYRQFNKMEHLVEHMKISYHSGHEPTCGVCKKHCRSFESLREHLIGPLPKQECKNIFSLRGCRFCMMILESPNA
RRIHQERCQFSSVNAGLTTRMAALGLRDKVMIDYTSSRSPKMVALSCKMVGGGSDGSLDVCARVCITDENDNVVFHTYVKPSMVVT
NYRYETTGIRPENLRDAMPLKHAQRKIQEFLCNGEPMWKIRPRGGKGRILVGHGLDHDPDRLQLEYPSSMMRDTAKYPPLMKTSKL
SNSLKYLTQAYLGYDIHVGIQDPYEDCVATMRLYTRMRYQKHKIEAYPLPADVQNRSNQVAWRQSEVERMSPNEMLSISRSDYYCW
CLDSLA
SEQIDNO: 155
ATGGAGTCATCGGAAACCCTAAGGAACAAGTGCGCAGCTTGTTATAGGCAATTCAACAAAATGGAACATTTGGTGGAACACATG
AAGATCTCTTATCACTCCGGTCATGAGCCTACTTGTGGAGTTTGCAAGAAACATTGCCGATCTTTTGAGTCCCTCCGCGAACATC
TCATAGGACCATTGCCAAAACAAGAATGCAAGAATATTTTTAGCCTTCGCGGATGCAGATTCTGCATGATGATCCTCGAAAGCC
CTAACGCTCGTAGGATTCATCAAGAGAGATGTCAATTTTCGAGCGTCAATGCTGGATTGACGACTCGTATGGCAGCCTTAGGCC
TAAGAGATAAAGTCATGATCGACTACACGTCATCACGGTCTCCAAAAATGGTTGCACTCTCTTGCAAGATGGTAGGAGGAGGAA
GCGACGGGTCGTTGGATGTATGCGCAAGGGTTTGCATAACGGATGAGAACGACAACGTTGTGTTCCATACGTACGTGAAACCG
TCAATGGTCGTGACGAACTATAGGTACGAGACGACTGGTATACGTCCAGAGAATCTGAGGGACGCAATGCCGTTGAAACATGC
ACAAAGAAAGATTCAAGAATTTCTTTGTAATGGAGAACCCATGTGGAAGATTCGTCCAAGAGGTGGGAAAGGGAGGATTCTCGT
GGGACATGGGCTGGATCACGATCCTGACCGCCTTCAACTTGAATATCCTTCTTCCATGATGAGGGATACTGCAAAATATCCTCC
GTTGATGAAAACAAGCAAGCTGAGCAATTCTCTCAAGTACTTAACCCAAGCCTATCTCGGGTATGATATTCATGTTGGGATACAA
GACCCATACGAAGATTGTGTAGCGACGATGAGGCTTTACACGAGAATGAGATATCAGAAACATAAGATTGAAGCATATCCTTTA
CCAGCCGACGTGCAGAACCGTAGCAATCAAGTGGCTTGGAGGCAGAGCGAGGTCGAAAGGATGTCTCCTAATGAAATGCTCTC
CATCTCTCGCTCCGACTACTATTGCTGGTGCTTGGACTCCCTCGCTTGA
SEQIDNO: 156
ATGGAGTCATCGGAAACCCTAAGGTAACCATTTTCATCCTAAGCGTGTTATATCAAGCGTTTTAATTGGAAAATAAATCCCAAAG
AAAAACTCAATGGAGTGGCATGCAATATCATACCCAAATTCTGTAGTCAAGTGATGAAAAACTAAAATTTAAAGTTTTTGCTAAAA
TATATTTGTATATATATGTAAAAAAATATTTATGTATATATGTACACGTGCAGGAACAAGTGCGCAGCTTGTTATAGGCAATTCAA
CAAAATGGAACATTTGGTGGAACACATGAAGATCTCTTATCACTCCGGTCATGAGCCTACTTGTGGAGTTTGCAAGAAACATTGC
CGATCTTTTGAGTCCCTCCGCGAACATCTCATAGGTAAACAAGAAGCTCTATATAAAGAGTGACATATAGTTTTATATATACGCTA
TGCATTTGTATGTTAATTTTTAATTGTGTAATGTCAATAGGACCATTGCCAAAACAAGAATGCAAGAATATTTTTAGCCTTCGCGG
ATGCAGATTCTGCATGATGATCCTCGAAAGCCCTAACGCTCGTAGGATTCATCAAGAGAGATGTCAATTTTCGAGCGTCAATGC
TGTACGTAATCGTATTAATGTCCTTATCTCAATACTCCTCTTCTCATATTTGCGTAACTAAACTTGTAAATTGACCGCATGAAAAAA
AAATTCTTATATAAATAATATAATATCATATGATTTGTCTTTTTTCAGGGATTGACGACTCGTATGGCAGCCTTAGGCCTAAGAGA
TAAAGTCATGATCGACTACACGTCATCACGGTCTCCAAAAATGGTTGCACTCTCTTGCAAGATGGTAGGAGGAGGAAGCGACG
GGTCGTTGGATGTATGCGCAAGGGTTTGCATAACGGATGAGAACGACAACGTTGTGTTCCATACGTACGTGAAACCGTCAATG
GTCGTGACGAACTATAGGTACGAGACGACTGGTATACGTCCAGAGAATCTGAGGGACGCAATGCCGTTGAAACATGCACAAAG
AAAGATTCAAGAATTTCTTTGTAATGGAGAACCCATGTGGAAGATTCGTCCAAGAGGTGGGAAAGGGAGGATTCTCGTGGGACA
TGGGCTGGATCACGATCCTGACCGCCTTCAACTTGAATATCCTTCTTCCATGATGAGGTGACATTTCTATAAAGAAGAAAAGTAA
AGAGGACACATATATATAACTAGATTCCTTATAGTGTTACTTGGTTTCTGGCAACATAACCCAGGTTTATGTGAGTTGTTTTATAA
TTAACGTGAATTTCTAATATCATTTTTATATATTGGTGTATGTAGGGATACTGCAAAATATCCTCCGTTGATGAAAACAAGCAAGC
TGAGCAATTCTCTCAAGTACTTAACCCAAGCCTATCTCGGGTAATATATCTACTTTTGTTTCTGTAATACTACTTTCGAATATAACA
TTAATTTGGAAAATGAATATAGATATATGCATGCTTTACGTGTATTGATACATAAGTGTATCATGTACTATCCATTAATTTTTTGGT
GATGTATGGGTATTGATGCTTTGTATGGGCTTAAAGCTAAAAGGAAGGGATTCAAAACGTGAAAAGATTCATATGTTATATAATAT
ATACCATTTTCTAACAAAAACAAAAAGAACTTACATAGTTGGAGATTTTTAATTTACATGAGATGCTTACTAGCGTAATTTTTATCG
CCAAGTTGACTACATCAACCTGTTTATTTCATTTGATTTCCTATCAATTACAAACAGTTCAACTAGTATACCATTGTTTTGTATGTA
AAACTTTAGGGATGAGTGTGTATGCCTAATAATATCATATGTTCCATATAATCTGTGTTCGTTTCTACACACCGTAGAAGTAATCT
CAAACACTTGAGATCAATTAATTATACCCATTATCTAATGTACAACAATATATATTAAGGATGGTATTGATGTGCTTATAGGTATGA
TATTCATGTTGGGATACAAGACCCATACGAAGATTGTGTAGCGACGATGAGGCTTTACACGAGAATGAGATATCAGAAACATAA
GATTGAAGCATATCCTTTACCAGCCGACGTGCAGAACCGTAGCAATCAAGTGGCTTGGAGGCAGAGCGAGGTCGAAAGGATGT
CTCCTAATGAAATGCTCTCCATCTCTCGCTCCGACTACTATTGCTGGTGCTTGGACTCCCTCGCTTGA
SEQIDNO: 157
MRTSYHSVHEPTCGICNKHCRSFDSLREHLIGPLPKQECKNIFSICGCRFCLTNLESPNARRIHQERCQFSNGLTTRMAVLGLRDNPTI
DYTSSRSPRVVALSCKMVGGGSDESLDLCARVCITDESENVIFHTYVKPTLPITNYRYETTGIRPENIRDAMPLKQAQRKIKEFLCYGE
PMWKIRPRSGKARILVGHGLDSHLDCLQIEYSSSMIRDTAEYPPLMKTSKLSNSLKYLTQAYLGYDIHVGMQDPYEDCVATMRLYKRI
RYQKHKTDAYPLASDTHNTNNYASWRQSELENMSEDELLNLSRSDYYCWCLDSVP
SEQIDNO: 158
ATGAGGACGTCTTACCATTCGGTTCATGAACCTACGTGTGGCATTTGCAACAAACACTGCCGATCTTTTGATTCCCTCCGTGAAC
ACCTCATTGGGCCATTGCCGAAACAGGAATGTAAGAACATTTTCAGCATCTGCGGCTGCAGATTCTGCCTTACGAACCTTGAAA
GCCCCAACGCTCGTAGGATCCATCAGGAGAGATGCCAATTCTCAAACGGACTAACTACTCGTATGGCGGTCTTAGGCCTAAGA
GACAATCCTACAATCGATTACACTTCTTCCAGGTCACCACGAGTGGTCGCACTCTCATGCAAGATGGTTGGAGGAGGGAGTGAT
GAATCGCTTGACCTATGCGCGAGAGTTTGCATAACAGATGAGAGCGAAAACGTGATTTTTCACACGTACGTGAAGCCAACATTG
CCCATAACGAATTACAGGTACGAGACTACAGGAATACGACCTGAGAATATAAGGGACGCGATGCCATTGAAACAAGCACAGAG
AAAGATTAAGGAGTTTCTCTGTTATGGAGAACCAATGTGGAAGATTCGTCCAAGAAGTGGAAAAGCGAGGATTCTCGTGGGACA
TGGACTTGATAGCCATCTTGACTGTCTTCAAATTGAATATTCTTCTTCCATGATAAGAGATACTGCGGAATACCCTCCATTGATGA
AAACAAGCAAGCTAAGCAACTCGCTCAAGTACTTAACCCAAGCCTATCTGGGGTATGACATTCATGTGGGGATGCAAGATCCTT
ATGAGGACTGTGTCGCGACGATGAGGCTGTACAAAAGAATACGATATCAAAAACACAAGACGGATGCTTATCCGTTGGCCTCAG
ACACGCATAACACAAATAACTATGCGTCCTGGAGACAGAGCGAGCTTGAGAATATGTCAGAGGATGAGTTGCTCAACCTTTCTC
GGTCAGACTATTACTGCTGGTGCTTGGACTCAGTTCCTTGA
SEQIDNO: 159
ATGAGGACGTCTTACCATTCGGTTCATGAACCTACGTGTGGCATTTGCAACAAACACTGCCGATCTTTTGATTCCCTCCGTGAAC
ACCTCATTGGTATATCTCCAACCTCTATATATCTACTCTCTGACTCTCTAATTTGGCTTTCTAATTTCATTTAAAATCATATTGTATG
TAGGGCCATTGCCGAAACAGGAATGTAAGAACATTTTCAGCATCTGCGGCTGCAGATTCTGCCTTACGAACCTTGAAAGCCCCA
ACGCTCGTAGGATCCATCAGGAGAGATGCCAATTCTCAAACGTCAGTTATGTAATTATACACATAAGTTAATATTTTCATAAATGA
ATTAACTACAGCATATTGCTATAGGTCTCGATTTCAGACACAAAACAAGCATTTGTGCATCTTTTTGTGTTAACAAGGGTGATTGG
AATGAACTCTAGCTTTATATTTTTGGCTGTAGAATTTAAGCTATAGATTTATTTGATGTAGATTATTTTGCTGTATCTTTGTAAAGC
AATATTTTTTCTTTGGAAATAAAATTCTATACACCCATATTTTAATTTTGTAGAAATATTTTTGTTGTGAATTTTTTAAGGAAATGAAA
ACTCGATTGGTTGACATATATGGTTCTAGAGTAAATTTTGGCTGTCTAGAGCATCTACAGCCGCATCCAACGTTTTCATTTTCTGA
TGAAATATTTTATTTTGTAAATAATTTTGCAAATGATAACTTAAATAAAAACACACTACTGTATTAGAAATAAATGAAATAAAACAGT
ATATTCTTTCTATTTGTTGCTTGATACTAATATCTAAGTAACATTTCAGGGACTAACTACTCGTATGGCGGTCTTAGGCCTAAGAG
ACAATCCTACAATCGATTACACTTCTTCCAGGTCACCACGAGTGGTCGCACTCTCATGCAAGATGGTTGGAGGAGGGAGTGATG
AATCGCTTGACCTATGCGCGAGAGTTTGCATAACAGATGAGAGCGAAAACGTGATTTTTCACACGTACGTGAAGCCAACATTGC
CCATAACGAATTACAGGTACGAGACTACAGGAATACGACCTGAGAATATAAGGGACGCGATGCCATTGAAACAAGCACAGAGA
AAGATTAAGGAGTTTCTCTGTTATGGAGAACCAATGTGGAAGATTCGTCCAAGAAGTGGAAAAGCGAGGATTCTCGTGGGACAT
GGACTTGATAGCCATCTTGACTGTCTTCAAATTGAATATTCTTCTTCCATGATAAGGTAAATATACCTTTTACTACACAATTTTTTT
GGATGGTAACGACAATTTTCACTCATACCACTGGTGCAGATTAGTCCGGATTTGATTCATAATATATGATCATTAAGAAATAGAAT
ATGTATGATAAGACATATTATGTTTATTTGTAAAGAGATACTGCGGAATACCCTCCATTGATGAAAACAAGCAAGCTAAGCAACTC
GCTCAAGTACTTAACCCAAGCCTATCTGGGGTAAATATTCTTCTTCTTTTTTTGCATCTCTTTTTTTCAATTTTACAAAATTGAAAG
ACCCATAAACATGAATGGGTACATTTTTGAAATTTAATTTCAGCTAATCTGTATATAATTTTAAAAATATTCGTCACAAGTTGGTAA
ATATAACTTTACTTAAGCAAAAAAAAAAAAATTCTGTAGTTCATCTGATCAAACCTACTATCAAAGATTCCATAACTCATAAAACAA
TTATTCAACTCGGAAAAGGTTTGTCTACTAAACCATGCATGTATACAGATTACACGTTTCTATAATTTTATTTTAATTTGCCGTGTG
TAATATTCGGACGCAACAACTATAAAGTAGGTATCCGATGTACTTATGACGTGATTAAAAGTTTTTAATCCGTGTAAAGTACAATA
TGCTGATTAGCTAAAAAGTTTCTTAAAAATTCGAATAAATCAACCGATGATAATCCTAGGAATATTTCGAATAACTGACGTCTCTA
CGATTTCTATGTCTCTACCTCATACTTCGTATCTTTTATTCATAACCAAAAGTTTCGAATTTCTAATTTGCTCCTCTCGTTTATAAAA
GGTTATCCTAAAGACACAAAACTACAAAATTAGATTATTATTTTTCCTAAAATATACAATGCAGTAAAAAAGTTCTGTATATTTTAA
ACTAAAGAAATATATTTCCTCTTTGTTCAGGATTACGGATTAGTGGCAGTGGCAGAAAATAACTTATAACATCACAACTAGACTAC
TTTAATAAAATTTTGATAGGAGTCAATATTCGTTTTTGGTTGGAGTCAACATAATTTATTTGGTAAAAATAAATTGTTTTCATAAACT
TTATGGGTGTCACTTGACCCCTTCATAACACACTCTCCGCCATTGATAGTGGTAGGCTCTAGAACTTATGGTTTCTAATATAGGC
TTCATTTCAGTGCATATGGTATTTTTATGGTTTAGGGTTTATAAATAATAAACTTTGAGGTCATAAAATAATACTCATTATATTCCTG
AAATTAAGATTTTCTAGAGTTAGAGTATGCACGCTTAATAAATATTTATAATTTAATTTATTTTTTACTTTATTATACACTTTCCAATA
ACTTTCTACCAATGAAATTTAATCAATTCAAATATTCTCAATTATTGTCCTCAAAAGTATAAAAAAGTACCTTAAGAATATAGAAAAT
CTATCTTTGTGTAACAAGAAAAAAATCTAAAAAATCTAAAAAATCTTACTTTCGGGAACAGAGGGAGTATTATGAATTGGATTTAC
AGTACATTATTAATTTATAAAAGTTTATGCTATTTCCTTATTTTGATCTAAAATAAGACTGTTTAAATTAAATCTCATTTCTATCTTTT
CCGACCTTCTAAAGGTATGACATTCATGTGGGGATGCAAGATCCTTATGAGGACTGTGTCGCGACGATGAGGCTGTACAAAAGA
ATACGATATCAAAAACACAAGACGGATGCTTATCCGTTGGCCTCAGACACGCATAACACAAATAACTATGCGTCCTGGAGACAG
AGCGAGCTTGAGAATATGTCAGAGGATGAGTTGCTCAACCTTTCTCGGTCAGACTATTACTGCTGGTGCTTGGACTCAGTTCCT
TGA
SEQIDNO: 160
MDYRSSMESSETLRNKCAACYRQFNKMEHLVEHMKISYHSGHEPTCGVCKKHCRSFESLREHLIGPLPKQECKNIFSLRGCRFCMM
ILESPNARRIHQERCQFSSVNAGLTTRMAALGLRDKAMIDYTSSRSPKMVALSCKMVGGGSDGSLDLCARVCITDENDNVVFHTYVK
PSMVVTNYRYETTGIRPENLRDAMPLKHAQRKIQEFLCNGEPMWKIRPRGGKGRILVGHGLDHDLDRLQLEYPSSMMRDTAKYPPL
MKTSKLSNSLKYLTQAYLGYDIHVGIQDPYEDCVATMRLYTRMRYQKHKIEAYPLPADVQNRSNQVAWRQSEVERMSPNEMLSISRS
DYYCWCLDSLA
SEQIDNO: 161
ACACACCTAGCATAATACATTAATAAAAAAACTCATCAAAAGAATTGTTTAACTTCTCCCCACTCTTAAATTGTTGAGTTCTTTGTT
TGCATTTTCATACGTACCATGGATTACAGATCATCTATGGAGTCATCGGAAACCCTAAGGAACAAGTGCGCAGCTTGTTATAGG
CAATTCAACAAAATGGAACATTTGGTGGAACACATGAAGATCTCTTATCACTCCGGTCATGAGCCTACTTGTGGGGTTTGCAAGA
AACATTGCCGATCTTTTGAGTCACTCCGCGAACATCTCATAGGACCATTGCCAAAACAAGAATGCAAGAACATTTTTAGCCTTCG
CGGATGCAGATTCTGCATGATGATCCTCGAAAGCCCTAACGCTCGTAGGATTCATCAAGAGAGATGCCAATTTTCGAGCGTCAA
TGCTGGATTGACGACTCGTATGGCAGCGTTAGGCCTTAGAGATAAAGCCATGATCGACTACACGTCATCACGGTCTCCAAAAAT
GGTTGCACTCTCTTGCAAGATGGTAGGAGGAGGAAGCGACGGGTCGTTGGATCTATGCGCAAGGGTTTGCATAACGGATGAGA
ACGACAACGTTGTGTTCCACACGTACGTGAAACCGTCAATGGTCGTGACGAACTATAGGTACGAGACGACCGGTATACGTCCA
GAGAATCTGAGGGACGCAATGCCGTTGAAACATGCACAAAGAAAGATTCAAGAATTTCTTTGTAATGGAGAACCCATGTGGAAG
ATTCGTCCAAGAGGTGGGAAAGGGAGGATTCTCGTGGGACATGGGCTGGATCACGATCTTGACCGCCTTCAACTTGAATATCC
TTCTTCCATGATGAGGGATACTGCAAAATATCCTCCGTTGATGAAAACAAGCAAGCTGAGCAATTCTCTCAAGTACTTAACCCAA
GCCTATCTCGGGTATGATATTCATGTTGGGATACAAGACCCATACGAAGATTGTGTAGCGACGATGAGGCTTTACACGAGAATG
AGATATCAGAAACATAAGATTGAAGCATATCCTTTACCAGCCGACGTGCAGAACCGTAGCAATCAAGTGGCTTGGAGGCAGAGC
GAGGTCGAAAGGATGTCTCCTAATGAAATGCTCTCCATCTCTCGCTCCGACTACTATTGCTGGTGCTTGGACTCCCTCGCTTAA
TTCCAAAAACTATGGGGTTAAAATTTGAGAATCTCTCATAATTACTTCATAAACTTTTTCGAGATTTTTAATTTAGTAGCGTAAAGT
CGAATAAGCATTACTTCTGATTTCAAATAGTATCTAAATTAATTTTATAGTATGTGTCGTGTGAGATGCGAATGTTGTTCCTGTACT
TCTAATAACGCATATTGCTCTTAATTATTAATAATTATT
SEQIDNO: 162
ACACACCTAGCATAATACATTAATAAAAAAACTCATCAAAAGAATTGTTTAACTTCTCCCCACTCTTAAATTGTTGAGTTCTTTGTT
TGCATTTTCATACGTACCATGGATTACAGATCATCTATGGAGTCATCGGAAACCCTAAGGTAACCATTTTCATCCTAAGCGTGTG
TTATATCAAGCGTTTTAATTGAAAAAAAAAATCCCAAAGAAAAACTCAATGGAGTGGCATGCAATATCATACCCAAATTCTGTGGT
CAACTGATGAAAAATTAAGACTAAAATTATTTTTTTTTCTAAAATATATTTGTATATATATGTTAAAAATATATTGATGTATATATGTA
CACGTACAGGAACAAGTGCGCAGCTTGTTATAGGCAATTCAACAAAATGGAACATTTGGTGGAACACATGAAGATCTCTTATCA
CTCCGGTCATGAGCCTACTTGTGGGGTTTGCAAGAAACATTGCCGATCTTTTGAGTCACTCCGCGAACATCTCATAGGTAAATA
AGAAGCTATATATAAAGAGTGACATATAGTTTTATATATACGCTATGCATTTGTATGTTAATTTTTAATTGTGTAATGTCAATAGGA
CCATTGCCAAAACAAGAATGCAAGAACATTTTTAGCCTTCGCGGATGCAGATTCTGCATGATGATCCTCGAAAGCCCTAACGCT
CGTAGGATTCATCAAGAGAGATGCCAATTTTCGAGCGTCAATGCTGTACGTAATCGTATTATTGCCCTTATCTCAATACTCCTCTT
CTCATATTTGCGTAACTAAACTTGTAAATTGACCACATGAAAAAAACAATTCTTATATAAATAATGTAATATCATATGATTTGTCTTT
TTCAGGGATTGACGACTCGTATGGCAGCGTTAGGCCTTAGAGATAAAGCCATGATCGACTACACGTCATCACGGTCTCCAAAAA
TGGTTGCACTCTCTTGCAAGATGGTAGGAGGAGGAAGCGACGGGTCGTTGGATCTATGCGCAAGGGTTTGCATAACGGATGAG
AACGACAACGTTGTGTTCCACACGTACGTGAAACCGTCAATGGTCGTGACGAACTATAGGTACGAGACGACCGGTATACGTCC
AGAGAATCTGAGGGACGCAATGCCGTTGAAACATGCACAAAGAAAGATTCAAGAATTTCTTTGTAATGGAGAACCCATGTGGAA
GATTCGTCCAAGAGGTGGGAAAGGGAGGATTCTCGTGGGACATGGGCTGGATCACGATCTTGACCGCCTTCAACTTGAATATC
CTTCTTCCATGATGAGGTGACATTTCTATAAAGAAGAAAAGTAAAGAGGACACATATATATAACTAGATTCCTTATAGTGTTACTT
GGTTTCTCGCAACACAACCCAGACTTTACGTGAGTTGTTTTACGATTAACGTGAATTTCTAATATCATTTTTATATATTGGTGTGT
GTAGGGATACTGCAAAATATCCTCCGTTGATGAAAACAAGCAAGCTGAGCAATTCTCTCAAGTACTTAACCCAAGCCTATCTCG
GGTAATATATCTACTTTTGTGTTTGTAATACAACTTTCGAATATAACATTAATTTGGAAAATGAATATAGATATATGCATACTTTAC
GTGTATTGATACATAAGTGTATCATGTACTATCCATTAATTTTTTTGGTGATGTATGGGTATTGATGCTTTGTATGGGCTTAAAGC
TAAAAGGAAGGGATTCAAAACGTGAAAAGATTCATATGTTATATAATATAACCATTTTCTAACAAAAACAAAAAGAATTTACATAGT
TGGAGATTTTTAATTTACATGAGATGCTTACTAGCGTAACTTTTATCTCCAAGTTGACTGAATCAACCTATTTATTTCATTTTATTTC
CTATCAATTACAAATAGTTCAACTAGTATACCATTGTTTTGTATGTAAAATTATAGGGATGAGTGTGTATGCCTAATATCATATGTT
CCATATAATCTGTGTTCGCACCGTAGAAGTAATCTAAAACACTTGAGATCAATTAATTATACCCATTATCTAATGTACAACAATATA
TATATATTAAGGATGGTATTGATGTGCTTATAGGTATGATATTCATGTTGGGATACAAGACCCATACGAAGATTGTGTAGCGACG
ATGAGGCTTTACACGAGAATGAGATATCAGAAACATAAGATTGAAGCATATCCTTTACCAGCCGACGTGCAGAACCGTAGCAAT
CAAGTGGCTTGGAGGCAGAGCGAGGTCGAAAGGATGTCTCCTAATGAAATGCTCTCCATCTCTCGCTCCGACTACTATTGCTG
GTGCTTGGACTCCCTCGCTTAATTCCAAAAACTATGGGGTTAAAATTTGAGAATCTCTCATAATTACTTCATAAACTTTTTCGAGA
TTTTTAATTTAGTAGCGTAAAGTCGAATAAGCATTACTTCTGATTTCAAATAGTATCTAAATTAATTTTATAGTATGTGTCGTGTGA
GATGCGAATGTTGTTCCTGTACTTCTAATAACGCATATTGCTCTTAATTATTAATAATTATT
SEQIDNO: 163
MDYRSSIESSETLRNKCAACYRQFNKMEHLVVHMKISYHSGHEPTCGVCKKHCRSFESLREHLIGPLPKQECKNIFSLRGCRFCMMI
LESPNARRTHQERCQFSSANAGLTTRMAALGLRDKAMIDYTSSRSPKVVALSCKMVGGGSDGSLDLCARVCITDESDNVVFHTYVK
PSMIVTNYRYGTTGIRPENLRDAMPLKHAQRKIQEFLCNGEPMWKIRPRGGRGRILVGHGLDHDLDRLQLEYPSSMMRDTAKYPPL
MKTSKLSNSLKYLTQAYLGYDIHVGIQDPYEDCVATMRLYTRMRYQKHKTEAYPQAADAQNRSNQVAWRQNEVERMSPDEMLSISR
SDYYCWCLDSLA
SEQIDNO: 164
ATGGACTACAGATCATCTATAGAATCATCAGAAACCCTAAGGAACAAGTGCGCAGCTTGTTATAGGCAATTCAACAAAATGGAAC
ATTTAGTGGTGCACATGAAGATCTCTTATCACTCCGGTCATGAGCCTACTTGTGGCGTTTGCAAGAAACATTGCCGATCTTTTGA
GTCCCTCCGGGAACATCTCATAGGACCATTGCCAAAACAAGAATGCAAGAACATTTTTAGCCTTCGCGGATGCAGATTCTGCAT
GATGATCCTGGAAAGCCCGAACGCTCGTAGGACCCATCAAGAGAGATGTCAATTTTCGAGCGCCAATGCTGGATTGACGACTC
GTATGGCGGCCTTAGGCCTAAGAGATAAGGCCATGATCGACTACACGTCCTCGCGGTCCCCAAAAGTGGTTGCACTCTCTTGC
AAGATGGTAGGAGGAGGAAGCGACGGGTCGTTGGATCTATGCGCAAGGGTCTGCATAACGGATGAGAGTGACAACGTTGTGT
TCCACACGTACGTGAAACCGTCAATGATCGTGACGAACTATAGGTACGGGACGACCGGGATACGTCCGGAGAATCTAAGGGAC
GCCATGCCGTTGAAACATGCTCAAAGAAAGATCCAAGAATTTCTTTGTAATGGAGAACCTATGTGGAAGATTCGTCCAAGAGGT
GGGAGAGGGAGGATTCTCGTGGGACATGGGCTCGACCACGATCTTGACCGCCTTCAACTTGAATATCCTTCTTCCATGATGAG
GGATACTGCAAAATACCCTCCGTTGATGAAAACAAGCAAGCTGAGCAATTCGCTCAAGTACTTAACCCAAGCCTATCTCGGGTA
TGATATTCATGTTGGGATACAAGACCCATACGAAGATTGTGTAGCGACGATGAGGCTGTACACAAGAATGAGATATCAGAAACA
CAAGACTGAAGCTTATCCTCAAGCTGCCGACGCACAGAACCGTAGCAATCAAGTGGCTTGGCGGCAGAACGAGGTCGAGAGG
ATGTCTCCTGATGAAATGCTCTCCATCTCTCGCTCCGACTACTATTGCTGGTGCTTGGACTCCCTCGCTTGA
SEQIDNO: 165
ATGGACTACAGATCATCTATAGAATCATCAGAAACCCTAAGGTAGCCATTTTTTCATCCTACGCGTGTTGTACCATGGATCATCG
TTTAAAATATTAAAAAAGAAGACTCATGGAGCGGCATGCAATATCATACCCAAATTCTATATGTGATAAAATGATTAAAACTAAAA
AAAAAGGTAAATATATTTATATATGTACACGTGCAGGAACAAGTGCGCAGCTTGTTATAGGCAATTCAACAAAATGGAACATTTA
GTGGTGCACATGAAGATCTCTTATCACTCCGGTCATGAGCCTACTTGTGGCGTTTGCAAGAAACATTGCCGATCTTTTGAGTCC
CTCCGGGAACATCTCATAGGTAAACACGAATATCTGTATATATATATAAAGATAAATATATCGTTTTTATATACACTATGAATTAGT
ATTTTTATOTTTAATTCTGTAATTAATGTCAATAGGACCATTGCCAAAACAAGAATGCAAGAACATTTTTAGCCTTCGOGGATGCA
GATTCTGCATGATGATCCTGGAAAGCCCGAACGCTCGTAGGACCCATCAAGAGAGATGTCAATTTTCGAGCGCCAATGCTGTAA
TCTTCGTAGTTCTTATCTCAACATACTTCACTTCTCATATTTGCGTATTTTAACTTGTAAAGAATCAACCACATGAAAGATATGTTC
TTACATATATATCATATGATTTCTTCCTTTCAGGGATTGACGACTCGTATGGCGGCCTTAGGCCTAAGAGATAAGGCCATGATCG
ACTACACGTCCTCGCGGTCCCCAAAAGTGGTTGCACTCTCTTGCAAGATGGTAGGAGGAGGAAGCGACGGGTCGTTGGATCTA
TGCGCAAGGGTCTGCATAACGGATGAGAGTGACAACGTTGTGTTCCACACGTACGTGAAACCGTCAATGATCGTGACGAACTA
TAGGTACGGGACGACCGGGATACGTCCGGAGAATCTAAGGGACGCCATGCCGTTGAAACATGCTCAAAGAAAGATCCAAGAAT
TTCTTTGTAATGGAGAACCTATGTGGAAGATTCGTCCAAGAGGTGGGAGAGGGAGGATTCTCGTGGGACATGGGCTCGACCAC
GATCTTGACCGCCTTCAACTTGAATATCCTTCTTCCATGATGAGGTAACATTTCTATAAGAAAAAGATAAAGAGGATACATACTTA
TAACTATATTTCTTTCAATATAACCTATATTTTTATGTTTTTTTTTTTTTTTTTTTTTTTTTTTTTTTTTTTTTTTGGCAACTAAATAAATGA
ATGCAGTTAAGGGTAATTGAACCCAGCATTTCTAGCACTGGTAATTTCTCTTAGAACCACTAGGCTAAAGTCACTTTTTTCCTATA
TTTTTATGTTAGCTTGTTTTTTTATCGTGGAATTTCTATATCATTTTGTATGTATATTGGTGTATGTAGGGATACTGCAAAATACCC
TCCGTTGATGAAAACAAGCAAGCTGAGCAATTCGCTCAAGTACTTAACCCAAGCCTATCTCGGGTAATATATCTAGTTTTGTTTTT
GTAATATTACTTTACTTTTGAGTCTAACATATTGGAAAGTGAATATAGATGCATGCATACGTTATGTATATTGATACATATGTGAAG
CAAATATTATCAATTAATTATTTCTCTTTTTTTTGCTAACTAATTAATTCTTTCTCAAAAAAATATCATCAATTAATTTATTAGGTGTT
AACTATTCATGCTTTGTATGGCTTAAAGTTATAAGGAAGAGAGGATTCAAAACGTGAAAGATTAACATGTTTTAAAATGTATTCAT
CGACATCTACATATATATTTTATATATATACCATATTATATATAGATTGTATGGCTAAACTATCCAACAAAAAATGAAACACTTTACA
TGGTTAATTAGACATTTTTATTTTACACGAGATGCTATTACCGTAAATTTTTCTCTCCAAATCGAATACATCAACCTGCTTATTTCG
TTTTTTTTTTTGCCAATCGCAAATTATTCAAATAGTACAACAGAGTTTTTTTTTTGTATTTTAAATACTATGGATGAGTGTGTATGCC
TCCTCATATCATATGTTGCATATAATATATGTTCGTGTCTACGCACCGAATAAGTAATATAAAACATTTGAGATCAATTAGACCCA
TTATCTGATGTTCAAGATTATTAACTAATGATGATTTTTGATGGTAATTATTTAGGAAACTTAACATTTACACCCAAAAAAAACTAA
TGATGATTATTGATGTGCTTATAGGTATGATATTCATGTTGGGATACAAGACCCATACGAAGATTGTGTAGCGACGATGAGGCTG
TACACAAGAATGAGATATCAGAAACACAAGACTGAAGCTTATCCTCAAGCTGCCGACGCACAGAACCGTAGCAATCAAGTGGCT
TGGCGGCAGAACGAGGTCGAGAGGATGTCTCCTGATGAAATGCTCTCCATCTCTCGCTCCGACTACTATTGCTGGTGCTTGGA
CTCCCTCGCTTGA
SEQIDNO: 166
MDCRSMESSETLRNKCAACYKQFNKMEHLVEHMKISYHSGHEPTCGVCKKHCRSFESLREHLIGPLPKQECKNIFSILGCRFCLMILE
TPNARRIHQERCQFSSVNAGLTTRMAALGIRDKDMIDYTSSRSPKVVALSCKMVGGGSDGSLDLCARVCITDEGDNVVFHTYVKPSM
AVTNYRYEKTGIRPENLRDAMPLKHAQRKIQEFLCNGEPMWKIRPRGGKGRILVGHGLDHDLDRLQLEYPSSMIRDTAKYPPLMKTS
KLSNSLKYLTQAYLGYDVHVGIQDPYEDCVATMRLYTRMRYQKHKIEAYPLAADAHNRSNQVVWRQNEFERMSPDEMLSISRSDYY
CWCLDSLA
SEQIDNO: 167
ATGGATTGCAGATCTATGGAGTCATCGGAAACCCTAAGGAACAAATGCGCAGCTTGTTATAAGCAATTCAACAAAATGGAACATT
TAGTGGAGCACATGAAGATCTCGTATCACTCCGGTCATGAGCCTACCTGCGGCGTTTGCAAGAAACATTGCCGATCTTTTGAGT
CCCTCCGAGAACACCTCATAGGGCCATTGCCAAAACAAGAATGCAAGAACATTTTCAGCATTCTCGGATGCAGATTCTGCTTGA
TGATCCTCGAAACCCCGAACGCTCGTAGGATCCATCAAGAGAGATGCCAATTTTCGAGCGTCAATGCGGGATTGACGACTCGT
ATGGCGGCCTTAGGCATAAGAGATAAGGACATGATCGACTACACGTCGTCTAGGTCCCCAAAAGTGGTTGCACTTTCTTGCAAG
ATGGTAGGAGGGGGAAGCGACGGGTCGCTGGATCTATGTGCAAGGGTTTGCATAACGGATGAGGGCGACAATGTTGTGTTCC
ATACGTACGTGAAACCGTCAATGGCCGTGACGAACTATAGGTATGAAAAGACCGGCATACGTCCGGAGAATCTAAGGGACGCA
ATGCCCTTGAAACATGCACAAAGAAAGATTCAAGAGTTTCTTTGTAATGGAGAACCCATGTGGAAGATTCGTCCAAGAGGTGGG
AAAGGGAGGATTCTCGTGGGACATGGCCTCGATCACGATCTTGACCGCCTTCAACTTGAATATCCTTCTTCCATGATAAGGGAT
ACTGCGAAATACCCTCCCTTGATGAAAACAAGCAAGCTGAGCAATTCCCTCAAGTACTTGACCCAAGCCTATCTCGGGTATGAT
GTTCATGTTGGGATACAAGACCCATACGAAGATTGTGTAGCGACGATGAGGCTTTACACGAGAATGAGATATCAGAAACACAAG
ATTGAAGCTTATCCTTTAGCTGCCGACGCGCACAACCGTAGCAATCAAGTGGTTTGGAGGCAGAACGAGTTCGAGAGGATGTC
TCCTGATGAAATGCTTTCCATCTCTCGCTCCGACTACTATTGCTGGTGCTTGGACTCCCTCGCTTGA
SEQIDNO: 168
ATGGATTGCAGATCTATGGAGTCATCGGAAACCCTAAGGTAACCTATCCTCCATCGTACGTGTATTAGATTATATCAAGCGTTTA
TATGAAAATATCGAAAAGAACTCAGGGAGGGTGTCATCCACTATCACATCCAAATTCTATAATCAATTGATGAAAAACTAGAAATA
ATTTGTTTTCCTTAAATTATAGTTTTATACATATGTACACGTACGTGCAGGAACAAATGCGCAGCTTGTTATAAGCAATTCAACAA
AATGGAACATTTAGTGGAGCACATGAAGATCTCGTATCACTCCGGTCATGAGCCTACCTGCGGCGTTTGCAAGAAACATTGCCG
ATCTTTTGAGTCCCTCCGAGAACACCTCATAGGTAAATGATAACCTATATATATTAAGAGAAATGTATCGTTTTATACATTCACGA
CGCATATGTGTATTTATTTTTAATTGTGTTATATACATAGGGCCATTGCCAAAACAAGAATGCAAGAACATTTTCAGCATTCTCGG
ATGCAGATTCTGCTTGATGATCCTCGAAACCCCGAACGCTCGTAGGATCCATCAAGAGAGATGCCAATTTTCGAGCGTCAATGC
GGTAATCTTTTCTTCTCATATTTGCATAATTAAAATTGTTAATAATCAACCGCACAAAATATATTTATTCTTACATATATGATATGAT
TTTCTCTGTTTCAGGGATTGACGACTCGTATGGCGGCCTTAGGCATAAGAGATAAGGACATGATCGACTACACGTCGTCTAGGT
CCCCAAAAGTGGTTGCACTTTCTTGCAAGATGGTAGGAGGGGGAAGCGACGGGTCGCTGGATCTATGTGCAAGGGTTTGCATA
ACGGATGAGGGCGACAATGTTGTGTTCCATACGTACGTGAAACCGTCAATGGCCGTGACGAACTATAGGTATGAAAAGACCGG
CATACGTCCGGAGAATCTAAGGGACGCAATGCCCTTGAAACATGCACAAAGAAAGATTCAAGAGTTTCTTTGTAATGGAGAACC
CATGTGGAAGATTCGTCCAAGAGGTGGGAAAGGGAGGATTCTCGTGGGACATGGCCTCGATCACGATCTTGACCGCCTTCAAC
TTGAATATCCTTCTTCCATGATAAGGTGGCATTTCTATAAAGAAAAAATATAAAGAGGATATATATATAATAGAAAAAGACTAGCA
TAGCACCAAACCAAGTTTTTCCTCAAACTAGCACTCAAGGATCAAAGTCACAAAAATAGGTTTCATTAAAAAGATAAATATACTCT
TAGGGTTTAGAGTTAGGGGTGGAGTTTTTGAATTAGAGTTTAAAATTTTATAAAATAAAAAATAAATACTAAAAAATTAAAAATAAA
AATTAAAAAAACAGTTTCAAAAAGTATTTTTGAATTCTAAAAAGAAAATTTAAAAAAAAAATAAAAAAAAATTCGAAAAAAAATTATA
AAAAATGTCGAATCTGAAAACATATAATCTGAAACTATAAAAAAAAATTTCATTTTTTTTATTTTTATTTTATTTGTTTTTATTTATTTT
GTTTGTTAATTTAATTTTAAACCAAAAGTATTAGACATATTTTATCATTTAATGAATGTCATTTTTGTGACTTTTTCCTTCTAATGTTA
TTTTTGAGATAAAAACTCAAAAGGTGCTATTATTGACAATTGTCACTAGATTCCTTTAGTGTTACTTGAAATCTTTCAATATAGCCA
ATGATTTTATGTGTGTTTTTTATATGACGGGATTTTGTATGATCTTATTGCTGTATGTAGGGATACTGCGAAATACCCTCCCTTGA
TGAAAACAAGCAAGCTGAGCAATTCCCTCAAGTACTTGACCCAAGCCTATCTCGGGTAATATATCTAATTTTGTTTTTGTAATAAT
ATTTTCAAACCGGGTGTTGATGCTTTGTATGGGCTTAAAGTTATAAGGGAGGGCTTCAAAACGTAAGATTCACATGTTTTCTAAT
ATATTCATTTCAAAATTTACACATAAACATATATATATAGTTTGTGTGACTAGAAATATCATTGAAGTAAATAAAATGAATTCATTAT
AATAAAAACAAATTTACATGGTTAGAAATTTTACTTTACCCTAGATACTTAATACTTATTAGCGTTGATTTTCCTTACATCAACCTG
GTTATTTCATTTTCTTTTCTTTTTAACGCAAATATATAGTTCCAATAGTATATACCCTTCTTTTCTGTATGTAATATTTTAAGATTTAT
TCCTAGGTTCACCCTTTAGGTTTACCAACTAATAAGATTTTGTTATTTCATATTCGATATOTTTCAAAAAAATGAGACAAAATATTA
TCAAATTATATTATGTTTTTAAAATAAAAAAGTAAAAAAAAAAAATAATAGTAGTTACAACAAAATAATTTAAAAAAATATTTTTAAC
GTCGTCAGTAAAACACTAAACCCTAAATGCTAAACCATAAACCATTGGATAACCCCTAAACCATTAGATAAATCCTAAACTCTAAA
TCAAAAACACTAAACACTAAACCCTAAATCCTAAATCCTTGAGTGTTTTAGTGTTTAGTGTTTTGATTTAGAAATTAATATTTATCC
AAGGGCTTAGAGTTTACCCAAATGTTTAGGATTTATATATGGATTAAGATTTAGAATTTAATGTTTTGCTGACGACGTTAAATATAT
ATATATATATTTTTTTTTTTTTTTTGTAACTACTACTATTTTAAATTTATTTATTTATTAATTTTTTATTTTTAAAACATAATATAATTTG
ACAATATTTTGTTTCATTTAAAAAAAATATCAAATATAAAATAACACAATTTTATTGGTTGATAAAGCTAGAGGTTTACTCTAGGGG
GTGAACCCAAAAATAAGTCATATTTTAGGGATGAGTAGTGTGTATGCCTCATACCATATGTTCAATATATAATATATGTTCGTGTC
CATCAACCGTATAAGTAATCTAAAACACTTGAGATCAATTATATACACATTAATTGTCTAATGTTCAAGAATCAAGACCATATATTA
ATGATGATTATTGATGTGCCTATAGGTATGATGTTCATGTTGGGATACAAGACCCATACGAAGATTGTGTAGCGACGATGAGGC
TTTACACGAGAATGAGATATCAGAAACACAAGATTGAAGCTTATCCTTTAGCTGCCGACGCGCACAACCGTAGCAATCAAGTGG
TTTGGAGGCAGAACGAGTTCGAGAGGATGTCTCCTGATGAAATGCTTTCCATCTCTCGCTCCGACTACTATTGCTGGTGCTTGG
ACTCCCTCGCTTGA
SEQIDNO: 169
MDYRLSGELSETQRNKCGGCYRQFNKKEHLVEHMRTSYHSVHEPTCGICNKHCRSFDSLREHLIGPLPKQECKNIFSICGCRFCLTIL
ESPNARRIHQERCQFSNVNYGLTARMAVLGLRDNPTIDYTSSRSPRVVALSCKMVGGGSDGSLDLCARVCITDESENVIFHTYMKPT
LPITNYRYETTGIRPENIRDAMPLKQAQRKIKEFLCYGEPMWKIRPRSGKARILVGHGLDSHLDCLQLEYSSFMIRDTAEYPPLMKTSK
LSNSLKYLTQAYLGYDIHVGMQDPYEDCVTTMRLYKRMRNQKHKTDAYPLASDTHNTNNYASWRQSELESMSEDELLNLSGSDYYC
WCLDSVP
SEQIDNO: 170
GTTGGCATAAACAAAAATAAACCCATCAAAGCAAACTCTATTGACACAAAAACTGCTTGCTCAATTTACAATGGACTACAGACTG
TCAGGGGAGCTCTCAGAAACCCAAAGGAACAAGTGTGGAGGGTGTTATAGGCAATTCAACAAGAAAGAACATTTGGTGGAACA
CATGAGGACGTCTTACCATTCGGTTCATGAACCTACATGTGGCATTTGCAACAAACACTGCCGATCTTTTGATTCCCTCCGTGAA
CATCTCATTGGGCCATTGCCGAAACAGGAATGTAAGAACATTTTCAGCATCTGCGGCTGCAGATTCTGCCTTACGATCCTTGAA
AGCCCCAACGCTCGTAGGATCCATCAGGAGAGATGCCAATTCTCAAACGTCAATTATGGACTAACTGCTCGTATGGCGGTCTTA
GGCCTAAGAGATAATCCTACAATTGATTACACTTCTTCCAGGTCACCACGAGTGGTCGCACTCTCATGCAAGATGGTTGGAGGA
GGGAGTGATGGATCGCTTGACCTATGCGCGAGAGTTTGCATAACAGATGAGAGCGAAAACGTGATTTTTCACACGTACATGAAG
CCAACATTGCCCATAACGAATTACAGGTACGAGACTACAGGAATACGACCTGAGAATATAAGGGACGCGATGCCATTGAAACAA
GCACAGAGAAAGATTAAGGAGTTTCTTTGTTATGGAGAACCAATGTGGAAGATTCGTCCAAGAAGTGGAAAAGCGAGGATTCTC
GTGGGACATGGACTTGATAGCCATCTTGACTGTCTTCAACTTGAATATTCTTCTTTCATGATAAGAGATACTGCGGAATACCCTC
CATTGATGAAAACAAGCAAGCTAAGCAACTCGCTCAAGTACTTAACCCAAGCCTATCTGGGGTATGACATTCATGTGGGGATGC
AAGATCCTTATGAGGACTGTGTCACGACGATGAGGCTGTACAAAAGAATGCGAAATCAAAAACACAAGACGGATGCTTATCCGT
TGGCCTCAGACACGCATAACACAAATAACTATGCGTCCTGGAGACAGAGCGAGCTTGAGAGTATGTCAGAGGATGAGTTGCTC
AACCTTTCCGGGTCAGACTATTACTGCTGGTGCTTGGACTCAGTTCCTTGA
SEQIDNO: 171
GTTGGCATAAACAAAAATAAACCCATCAAAGCAAACTCTATTGACACAAAAACTGCTTGCTCAATTTACAATGGACTACAGACTG
TCAGGGGAGCTCTCAGAAACCCAAAGGTACTTTTTTGTGCTATTGTGTTAAATTTTGGTTTTCTTTGGATATAAGTTATACCATCT
TCGATCAGCTGATTGAATAAGGTTATTTACACCCAAAAAAAAGATTGAATAAGCTTTCCTTTCTCACATGTATGTTTTACTACTAC
CATCAACATCCAATAAATTTGGAACATAAACTATAAGTAACTTAAGTTAGGTAAAAGAAATAGGACAATCAATACACAAATCTGTA
ACACCTAAACTTATATTTACATTCTTAGAAAAACTAAATGTACCGTAAGAATACTATAAAATTGTGTATTTAGTGTGAAATAAAATA
ATTGTGAAACAAATATCACTAATCATATTCGCCCATATAGAGGCTTTAAGTAGTAGGAAAACTACTTTCGACACCATACCTCTCCA
ACATCTCTGAAGCACACAGCCATGTATCATCAGCTTTAATGGCAGAAGCCATTGCTGTCCATCGAGCGGTTTCTCTAGCCGTTTA
TTCAAACGTCCGATCCCTGGCGGTTCTATCCGATTCCTTATCTCTGATCAAGCTCTTGAAGAAGGGATGGTATCAACCTGAACTG
TTCGGTATCATGTTTGATATCTATCACTTTATGTCTTTCTTTGATGTTATCACCTTTGATTTTATTTCTTGAAACTTCAACTCTGAGG
CTGATTCTGTGGCAAAATCAGTGCTTGCTATGTCTGTAACCCACTCCACTGTTGGAGTGTAGAACCCCCTTTAAGTAATGCAATG
CTTTGTTTGATCAAAAAAAAAAAAAGTAGTAGGAAAACTAGACTGTTAATGTACCCCATCAAATCATCTGGATTTTGATTTGGAAA
TCCTCTAGTTATCAAAAAGAAAAATTGCGAGATAAACAATGATAAAAAAGTATGCATGGATGCAGGAACAAGTGTGGAGGGTGTT
ATAGGCAATTCAACAAGAAAGAACATTTGGTGGAACACATGAGGACGTCTTACCATTCGGTTCATGAACCTACATGTGGCATTTG
CAACAAACACTGCCGATCTTTTGATTCCCTCCGTGAACATCTCATTGGTATATCTCTAACCTCTATATATCTACTCTCTGACTCTC
TAATTTGGCTTTCTAATTTCATTTAAAATCATATTGTATGTAGGGCCATTGCCGAAACAGGAATGTAAGAACATTTTCAGCATCTG
CGGCTGCAGATTCTGCCTTACGATCCTTGAAAGCCCCAACGCTCGTAGGATCCATCAGGAGAGATGCCAATTCTCAAACGTCAA
TTATGTAATTATACACATAAGTTAATATTTGCATAAATGAATTAACTACAGCATATTGCTATAGGTCTCGATTTCAGACACAAAACA
AGCATTTTGTGCATCTTTTTGTGTTTACTCAGGTGATTGGGATGAACTCTAGCTTTATATTTTTGGCTATAAAATTTAAGTTGTAGA
TTTATGTGATGTAGATTATTTTTCTGTATATTTGTAAAGCATTTTTTCTCTGGAAATAAAGCTTTATATACCCACATTTTAATTTTGC
AGAGTTTTTTTTGTTGTGAGTTTTTGAAAGAAATGAAAGCTCGATTGGTTGACATATATGACTCTAGACTAAATTTTGGCTGTCTA
GAACATTTACAGCATCAACCAACGATTTCATTTTGTGATGAAATATTTTATTTTGTAAATAATTTTGCAAATGATAACTTAAATAAAA
ACACAATACTGTATTAGACATAAATGAAATAAAACAGTATATTCTTTCTATTTGTTGCTTGATACTAATATCTAAGTAACATTTCAG
GGACTAACTGCTCGTATGGCGGTCTTAGGCCTAAGAGATAATCCTACAATTGATTACACTTCTTCCAGGTCACCACGAGTGGTC
GCACTCTCATGCAAGATGGTTGGAGGAGGGAGTGATGGATCGCTTGACCTATGCGCGAGAGTTTGCATAACAGATGAGAGCGA
AAACGTGATTTTTCACACGTACATGAAGCCAACATTGCCCATAACGAATTACAGGTACGAGACTACAGGAATACGACCTGAGAA
TATAAGGGACGCGATGCCATTGAAACAAGCACAGAGAAAGATTAAGGAGTTTCTTTGTTATGGAGAACCAATGTGGAAGATTCG
TCCAAGAAGTGGAAAAGCGAGGATTCTCGTGGGACATGGACTTGATAGCCATCTTGACTGTCTTCAACTTGAATATTCTTCTTTC
ATGATAAGGTAAATATACCTTTTACTACACAATTTTTTTGGATGGTAACGACAATTTTCACTCGTACCACTGGTGCAGATTAGTCC
GAATTTGATTCATAATATATGATCATTAATAAATAGAATATGTATGATAAGACATATTATGTTTATTTGTAAAGAGATACTGCGGAA
TACCCTCCATTGATGAAAACAAGCAAGCTAAGCAACTCGCTCAAGTACTTAACCCAAGCCTATCTGGGGTAAATATTCTTCTTCT
TTTTTGCATCTCTTTTTTCTTTCAATTTTACAAAATTGAAAGACCCATAAACATGAATGGGTACATTTTTGAAATTTAATTTCAGCTC
ATCTGTATATAATTTTAAAAATATTCGCCACAAGTTGGTAAATATAACTTTACTTAAACAAAAATTCTGTAGTTCATCTGATCAAAC
CTACTATCAAAGATTCCATAATTCATAAAACAATTATTCAACTCGGAAAAGGTTTGTCTACTAAACCATGCATGTATACAGATTAC
AAGTTTCTATAATTTTATTTTAATTTGCTGTGTGTAATATTCGGACGTAACAACTATAAAGTAGGTATCCGATGTACTTATGACGTG
ATTAAAAGTTTTTAATCCGTGTAAAGTACAATATGCTGATTAGCTAAAAAGTTTCTTAAAAATTCGAATAAATCAACCGATGATAAT
CCTAGGAATATTTCGAATAACTGATATCTCTACGATTTCTATGTCTCTACCTCATACTTCGTATCTTTTATTCATAACCAAAAGTTT
CGAATTTCTAATTTGCTCCCCTCGTTTTATAAAGGTTATCCTAAAGACACAAAACAAAAAAAAATAGATTATTATTTTTCCTAAAAT
ATACAATGCAGTAAAAAATTTCTGTATATTTTAAACTAAAGAAATATATTTCATCTTTGTTCAGGATTTCGGATTAGTGGCAGTGAC
AGAAAATAACTTATAACATCACAACTAGACTACTTTAATAAAATTTTGATAGGGGTCAATATTCTTTTTTGGTTGGAGTCAACATAA
TTTATTTGGTAAAAATAAATTGTTTTCATAAACTTTATGGGTGTCACTTGACCCCTTCATAACACACTCTCCGCCATTGATTAGTG
GTAGGCTCTGTAAGAAAGAGGAAAGCTTGAAGCTTTACTTAAGTGATGAACAAAGCTTTACTCAAATAAACATACTTNNNNNNNN
NNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNTCTGCAACTTCGGTTTATGTTGATCATGGGCCTCAGGCCCATCTCTGTTATTAGGCT
CTAGAAGTTATGGTTTCTAATATAGGCTTCATTTTCAGTGCATATGGTATTTTTATGGTTTAGGGTTTATAAATAATAAACTTTGAG
GTCATAAAATAATATTAGGAATTGGATTTACAGTACATTATTAATTTATAAAAGTTTATGCTATTTCCTTATTTTGATCTAAAATAAG
ACTGTTTAAATTGATCTCATTTCTATCTTTTCCGACCTTCTAAAGGTATGACATTCATGTGGGGATGCAAGATCCTTATGAGGACT
GTGTCACGACGATGAGGCTGTACAAAAGAATGCGAAATCAAAAACACAAGACGGATGCTTATCCGTTGGCCTCAGACACGCATA
ACACAAATAACTATGCGTCCTGGAGACAGAGCGAGCTTGAGAGTATGTCAGAGGATGAGTTGCTCAACCTTTCCGGGTCAGAC
TATTACTGCTGGTGCTTGGACTCAGTTCCTTGA
SEQIDNO: 172
MDCRIESAETHRNKCAACFRQFNKLEHLVEHMRISYHSVHEPTCGICRKHCRSFESLREHLIGPLPKQECRDIFSYRGCKFCLKVFES
PNSRRIHQEKCQLSGTNAGIIGRFSNLGLRDNLAIGGGARGPQVVALACKMVGGGSDGSLDLCARVCLIDEHENIIFHSYVKPPIPVAN
YRYETTGITPEYLRDAMPMRHVQRRIHDFLCNGEPMWTIRARGGRARILVGHGLDHDLESLQIEYRAEKIRDTAKYPPLMKTSKLSNS
LKYLTQAYLGYDIQTGIQDPYEDCIATMRLYMRMRSQAHRVQEYPLASDPQNRNNFASWRQSEIERMSPEQMLEISRSDYYCWCLD
SLY
SEQIDNO: 173
TGAATATTGCCTTTAGGAATCGTCCATTTATGCAAGTTGGAGCAATAGTACCTCTACCCTTTCCCTATAAATATCACCCCACTCCT
ACCTTTGACCTCAAGTCCAAGTCTTTTCTATTCTATTTCTTACGTAATAATTATATAGATTAGTATAAAGAACTATGGATTGCAGAA
TAGAGAGTGCCGAAACTCACAGGAATAAGTGTGCAGCATGCTTCCGACAGTTCAACAAACTGGAGCATCTTGTGGAGCACATG
AGGATCTCATACCATTCGGTTCATGAACCAACCTGTGGCATTTGCAGGAAACACTGCAGGTCTTTTGAGTCTCTCAGGGAACAT
CTTATAGGTCCATTGCCAAAACAGGAATGCAGAGATATATTTTCCTATAGAGGGTGCAAGTTTTGTTTGAAAGTCTTTGAAAGCC
CTAACTCTCGCAGGATCCACCAAGAAAAATGCCAACTCTCTGGAACAAATGCTGGAATAATTGGTCGCTTTTCAAACTTGGGACT
TCGTGATAATTTGGCTATTGGTGGTGGAGCAAGAGGACCACAAGTAGTTGCTCTAGCATGTAAAATGGTTGGAGGCGGCAGTG
ATGGCTCACTTGATCTCTGTGCAAGAGTTTGCTTAATCGATGAACATGAGAACATAATATTCCATTCTTATGTGAAGCCACCAATT
CCTGTCGCAAACTACAGGTATGAGACAACAGGCATCACACCAGAATATCTGAGGGATGCAATGCCAATGAGACATGTTCAGAG
GAGGATTCATGACTTCCTTTGCAATGGTGAACCTATGTGGACAATTCGAGCAAGAGGTGGAAGAGCCAGGATTCTTGTGGGTCA
TGGTTTGGATCATGACCTTGAAAGTTTGCAAATAGAATATCGAGCTGAAAAAATAAGGGACACTGCAAAATACCCTCCACTGATG
AAAACAAGCAAGCTGAGCAACTCACTCAAGTACTTAACACAGGCATATCTTGGGTATGACATTCAAACTGGGATTCAGGATCCTT
ATGAGGATTGTATTGCAACGATGAGGCTCTACATGAGAATGAGATCTCAAGCACATAGAGTACAGGAATACCCTTTGGCGTCTG
ACCCTCAGAACAGGAATAATTTTGCTTCATGGAGGCAAAGTGAGATTGAAAGAATGAGTCCTGAACAAATGCTAGAAATTTCAAG
GTCTGACTACTACTGCTGGTGCTTGGATTCCTTGTATTGATCCCTGAATGGTGAAAGCCAAGTACACCAATCAGAAAGCCCATG
AACATCAGGGAATTGATCAGTACACTACTAATTTAACCGATACAAATATCTAGTATGCAAAAATAAAGTACACCAAACAGTTGTCT
CTTTTTTACTAGTCAATAATTTCTTATTT
SEQIDNO: 174
TGCAAGTTGGAGCAATAGTACCTCTACCCTTTCCCTATAAATATCACCCCACTCCTACCTTTGACCTCAAGTCCAAGTCTTTTCTA
TTCTATTTCTTACGTAATAATTATATAGATTAGTATAAAGAACTATGGATTGCAGAATAGAGAGTGCCGAAACTCACAGGAATAAG
TGTGCAGCATGCTTCCGACAGTTCAACAAACTGGAGCATCTTGTGGAGCACATGAGGATCTCATACCATTCGGTTCATGAACCA
ACCTGTGGCATTTGCAGGAAACACTGCAGGTCTTTTGAGTCTCTCAGGGAACATCTTATAGGTCCATTGCCAAAACAGGAATGC
AGAGATATATTTTCCTATAGAGGGTGCAAGTTTTGTTTGAAAGTCTTTGAAAGCCCTAACTCTCGCAGGATCCACCAAGAAAAAT
GCCAACTCTCTGGAACAAATGCTGGAATAATTGGTCGCTTTTCAAACTTGGGACTTCGTGATAATTTGGCTATTGGTGGTGGAG
CAAGAGGACCACAAGTAGTTGCTCTAGCATGTAAAATGGTTGGAGGCGGCAGTGATGGCTCACTTGATCTCTGTGCAAGAGTTT
GCTTAATCGATGAACATGAGAACATAATATTCCATTCTTATGTGAAGCCACCAATTCCTGTCGCAAACTACAGGTATGAGACAAC
AGGCATCACACCAGAATATCTGAGGGATGCAATGCCAATGAGACATGTTCAGAGGAGGATTCATGACTTCCTTTGCAATGGTGA
ACCTATGTGGACAATTCGAGCAAGAGGTGGAAGAGCCAGGATTCTTGTGGGTCATGGTTTGGATCATGACCTTGAAAGTTTGCA
AATAGAATATCGAGCTGAAAAAATAAGGGACACTGCAAAATACCCTCCACTGATGAAAACAAGCAAGCTGAGCAACTCACTCAA
GTACTTAACACAGGCATATCTTGGGTATGACATTCAAACTGGGATTCAGGATCCTTATGAGGATTGTATTGCAACGATGAGGCTC
TACATGAGAATGAGATCTCAAGCACATAGAGTACAGGAATACCCTTTGGCGTCTGACCCTCAGAACAGGAATAATTTTGCTTCAT
GGAGGCAAAGTGAGATTGAAAGAATGAGTCCTGAACAAATGCTAGAAATTTCAAGGTCTGACTACTACTGCTGGTGCTTGGATT
CCTTGTATTGATCCCTGAATGGTGAAAGCCAAGTACACCAATCAGAAAGCCCATGAACATCAGGGAATTGATCAGTACACTACT
AATTTAACCGATACAAATATCTAGTATGCA
SEQIDNO: 175
GACGAGAGTTTATGGTTGGTTTGTTTGCAACAATATTATGAAATTCATATTAAAACAACGTGACAGCCGAAGTCAATGAAAGAAA
AGAGAAATAATGGGCCCAAGAATCTGGAACATACATAGATCATTACGAATGCAATAATGCTATACGCTACCAACAAACTGCACGA
CAAGATAGAGGAGAGGAAGCATGGCCAAAGAAAAAGAAAAAAGTAGAGGAGGAATCGGAAATTAATATACTGAATATTGCCTTT
AGGAATCGTCCATTTATGCAAGTTGGAGCAATAGTACCTCTACCCTTTCCCTATAAATATCACCCCACTCCTACCTTTGACCTCA
AGTCCAAGTCTTTTCTATTCTATTTCTTACGTAATAATTATATAGATTAGTATAAAGAACTATGGATTGCAGAATAGAGAGTGCCG
AAACTCACAGGTTTTCTTAATTTCTTACATCCATGCACAAGCGCACACATGCATACATCACATATGAACGAGTGCATGTGCTTCT
ATACTATTAAACATTGAATTGCATAAAATTAAAAATGAATTATAATCAACAGTGCATGGATATGTATAAATAACAATCATGTTTTTTT
TTTTTTTTTTTGCAATAGGAATAAGTGTGCAGCATGCTTCCGACAGTTCAACAAACTGGAGCATCTTGTGGAGCACATGAGGATC
TCATACCATTCGGTTCATGAACCAACCTGTGGCATTTGCAGGAAACACTGCAGGTCTTTTGAGTCTCTCAGGGAACATCTTATAG
GTAATACTCATTCATACGATGATTAATTGTGAAGCAAATTAAGTGCATGCCCTTAGCATATATCTTCCATGAAGAATATTTATCAT
CTGATTGTGAATTTGTGATGAAACTTAATCTGCTTTTCCAGGTCCATTGCCAAAACAGGAATGCAGAGATATATTTTCCTATAGAG
GGTGCAAGTTTTGTTTGAAAGTCTTTGAAAGCCCTAACTCTCGCAGGATCCACCAAGAAAAATGCCAACTCTCTGGAACAAATG
CTGTATAACTTCTAACTATGAATCTAAAGAATTTACAAATGATTATCCTAGTAAAAATTTATTCTAATAATTTAAAAACATAAAGTAA
TATTTTTTTATTAAAAATTTGAAAAGATATTAGAATTATTAATTGCGAATTTTTAACTTTTTGTGCACATTATTACAGGGAATAATTG
GTCGCTTTTCAAACTTGGGACTTCGTGATAATTTGGCTATTGGTGGTGGAGCAAGAGGACCACAAGTAGTTGCTCTAGCATGTA
AAATGGTTGGAGGCGGCAGTGATGGCTCACTTGATCTCTGTGCAAGAGTTTGCTTAATCGATGAACATGAGAACATAATATTCC
ATTCTTATGTGAAGCCACCAATTCCTGTCGCAAACTACAGGTTAATTGGACAATTAAACTTCCTTTATATTAGGCCTTAAAGATTT
AATTTAACCATACATTTTTTTTAATTGATAAGCAGTTTAACCATAATATATACCTAACGAAGCTGCTGATTCATTAACAGGTATGAG
ACAACAGGCATCACACCAGAATATCTGAGGGATGCAATGCCAATGAGACATGTTCAGAGGAGGATTCATGACTTCCTTTGCAAT
GGTGAACCTATGTGGACAATTCGAGCAAGAGGTGGAAGAGCCAGGATTCTTGTGGGTCATGGTTTGGATCATGACCTTGAAAG
TTTGCAAATAGAATATCGAGCTGAAAAAATAAGGTAAATAGTTCAATTTAACATTTAGGCAGAACACATGCAGCATAGGTATACAC
TCTCATACATATCTCAAATTAAATAATTAATCTGTTCAGAAATTTTTGTAACTTTGTATAAGGCAAGTAAAAGAACACTTTTTATTAG
ATCCTAACTACGGTTATTTATTTGTTTATTTATATTAGCTCCTCGATCTCTAGAATCAACAACAAAGATGGATGCCATACATCAAAT
CATAGGTTTATTTCAACACATTCAAATATTGATTTTGAATTCATATTGAATAATCAGGGACACTGCAAAATACCCTCCACTGATGA
AAACAAGCAAGCTGAGCAACTCACTCAAGTACTTAACACAGGCATATCTTGGGCAAGTATCCTTCACATTTTTCTTTACTGTAAAA
TAGAAGATGCATGTATGTTGAATTCATGATATATGTTTTAATATTAATTAATTAACGAGGCTTCTGTTTTGGGGCACATATACATAT
AAGCAGGTATGACATTCAAACTGGGATTCAGGATCCTTATGAGGATTGTATTGCAACGATGAGGCTCTACATGAGAATGAGATC
TCAAGCACATAGAGTACAGGAATACCCTTTGGCGTCTGACCCTCAGAACAGGAATAATTTTGCTTCATGGAGGCAAAGTGAGAT
TGAAAGAATGAGTCCTGAACAAATGCTAGAAATTTCAAGGTCTGACTACTACTGCTGGTGCTTGGATTCCTTGTATTGATCCCTG
AATGGTGAAAGCCAAGTACACCAATCAGAAAGCCCATGAACATCAGGGAATTGATCAGTACACTACTAATTTAACCGATACAAAT
ATCTAGTATGCAAAAATAAAGTACACCAAACAGTTGTCTCTTTTTTACTAGTCAATAATTTCTTATTTTATAAATAATACTCAACTTA
AGCAGTAGCTTACGCTACTACCATAGCTAGCACCCAGTGATTAAGGTACG
SEQIDNO: 176
MDAEADPPQNPITRHKCLACYKQYKKKEHLIEHMKTSYHSVHQPRCGVCQKHCKSFESLREHLTGPLPRGICSKIFSQQGCQLCLAL
FDSPGSLIDHRKICRISAPTCPGTSALPYIDSQFDCQDFSDENHAGEGPGGAVAMDCEMVGGGSDGSLELCARVCLVDEDERLIFHT
YVQPEIPVTNYRYDITGLTEEHLRNAMPLKEVREKLLQILHNGESIGKVRLDGGKARLLVGHDLAHDLDCLKMNYPDHMLRDTAKYRP
LMKTNLVSHSLKYLTRTYLGYDIQSGTHDPYEDCISVMRLYKRIRSQLHPEEDHGTMTLSNNIVGMPDSWISRELDNLTPDELYAMSR
SDYKCWCLDLIPRLSA
SEQIDNO: 177
ACTTGAAACAGTCGCTGCTGCGTCTTCTTCATCCGTGGTGTTAGGGTTTTGGTCGGAGTAGCCTTTTGCTTCGTCCAATATTTTG
GACATGGACGCCGAAGCGGACCCTCCTCAAAACCCTATCACAAGACACAAATGCTTGGCATGCTATAAGCAATATAAGAAGAAA
GAGCATCTTATTGAGCACATGAAAACCTCGTATCATTCTGTTCATCAGCCAAGATGTGGGGTATGTCAAAAGCACTGCAAATCTT
TTGAGTCTCTGAGGGAACATCTTACTGGTCCTTTGCCAAGAGGAATTTGTTCAAAGATTTTCTCTCAACAGGGCTGTCAACTTTG
CCTGGCACTATTTGATAGTCCCGGGTCTCTCATTGATCATAGAAAAATATGTCGCATATCTGCCCCTACTTGTCCAGGAACAAGC
GCATTGCCCTATATTGATTCCCAGTTTGATTGTCAAGATTTTTCTGATGAAAACCATGCTGGCGAGGGCCCTGGAGGAGCAGTT
GCAATGGACTGTGAAATGGTTGGTGGTGGAAGTGATGGTTCTCTGGAACTTTGTGCTAGAGTGTGTTTGGTTGATGAAGATGAG
AGATTAATCTTCCATACTTATGTACAGCCTGAAATACCTGTTACTAATTACAGATATGATATAACTGGATTGACAGAAGAGCATCT
TAGAAATGCCATGCCACTTAAGGAAGTTCGAGAAAAGCTACTGCAAATTCTACACAATGGAGAATCCATTGGCAAAGTTAGACT
GGATGGTGGAAAAGCAAGGCTTCTTGTGGGGCATGACTTAGCACACGATTTGGATTGTTTAAAAATGAATTATCCTGATCATATG
CTGAGAGACACTGCAAAGTACCGTCCGTTGATGAAAACCAACTTGGTCAGCCATTCACTCAAGTATCTCACCCGAACATATCTT
GGTTATGATATCCAATCCGGCACTCATGACCCTTATGAAGATTGTATTTCTGTCATGAGACTATACAAGAGAATACGATCTCAAC
TTCATCCGGAGGAAGACCATGGAACAATGACCCTGAGTAACAACATTGTTGGCATGCCTGATAGCTGGATATCTAGGGAACTTG
ACAACCTCACACCGGATGAACTTTATGCCATGTCTAGATCAGATTATAAGTGTTGGTGCTTGGATTTGATACCAAGATTGTCAGC
CTGAATTATTGATCTTTGCATTTGTCCATAACATATCATAAGCATATTTCTTCAAGAACTATGAGAATGGAGCATTTGCAATGATAT
TTTCTGTGCATATCTTCCTATGCATCGAGTTATCTTCCAGACAAGAAAATTACGAGAAAAGATAGGTGCTTTATGATGGAGCTTTT
GTAAATATTTAGAACGCTTTGTTCAAATCACATGCCCTTTTTTAGAATGAAGACTT
SEQIDNO: 178
ACTTGAAACAGTCGCTGCTGCGTCTTCTTCATCCGTGGTGTTAGGGTTTTGGTCGGAGTAGCCTTTTGCTTCGTCCAATATTTTG
GACATGGACGCCGAAGCGGACCCTCCTCAAAACCCTATCACAAGGTCAATTTTTGCTCCCTCACTTTAATTTTTTTTTTTTCCATG
ATGATCACCGTGTTGCTTCTGGAGGTAGCCGTTGATCGGAAATTCAAACATTGTCATAAAATAAATTAGGAAAAATGTTAAAATC
AAACTCATTTAATTTTTAAATTGGTTATTACTGCACTTTAGAGGACTCCGTCTCTCATGTGAAAGAATATTCTAGTTACTTTTCTCA
GCCCGTTTGTGGTCACTTTTTTTTTTTTTTTATTCCCTTGGGTATAATTGTTTATTTGCTTAGTTTCTTTGAAGAATACTAACTTATT
TCTCCCTACAGACACAAATGCTTGGCATGCTATAAGCAATATAAGAAGAAAGAGCATCTTATTGAGCACATGAAAACCTCGTATC
ATTCTGTTCATCAGCCAAGATGTGGGGTATGTCAAAAGCACTGCAAATCTTTTGAGTCTCTGAGGGAACATCTTACTGGTGAGTC
ATTTGTGCATTCAATCTCTGATGTCTACAGTATTGGTTTCATGTTTTAGCTCATTATTTATCCTTTTTCGTGCCTCTTTTCTTCCAT
GTTTTAGGTCCTTTGCCAAGAGGAATTTGTTCAAAGATTTTCTCTCAACAGGGCTGTCAACTTTGCCTGGCACTATTTGATAGTC
CCGGGTCTCTCATTGATCATAGAAAAATATGTCGCATATCTGCCCCTACTTGTCCAGTAAGTTGATTATTGGCAACGTAAAGCAT
TTATATTTATATTTAAAATAAAATGTCTTATGTGTGTTATTCACCCTAAGTAATCAAAGAAACCTGCTTACAATCATTGTTTCTTGTC
TACTCTTTTTATGTTCTTATTAGGAAGTTTATGTTGTGTGTGGAACTGTTGCAGGGAACAAGCGCATTGCCCTATATTGATTCCCA
GTTTGATTGTCAAGATTTTTCTGATGAAAACCATGCTGGCGAGGGCCCTGGAGGAGCAGTTGCAATGGACTGTGAAATGGTTGG
TGGTGGAAGTGATGGTTCTCTGGAACTTTGTGCTAGAGTGTGTTTGGTTGATGAAGATGAGAGATTAATCTTCCATACTTATGTA
CAGCCTGAAATACCTGTTACTAATTACAGGTACTTTTTTGCTCTATTTCTCTTTTATGTCTATCCTTCCTATTTTTTACTCTTTCTTT
CACTTCTAATCCACACAAAGCATTATTGTCAAAGAGAAAAGTGGACAAGGAAAAAAGAGTGCTTGGGTTGATTTTGTTTGTTATTT
TCAGATATGATATAACTGGATTGACAGAAGAGCATCTTAGAAATGCCATGCCACTTAAGGAAGTTCGAGAAAAGCTACTGCAAAT
TCTACACAATGGAGAATCCATTGGCAAAGTTAGACTGGATGGTGGAAAAGCAAGGCTTCTTGTGGGGCATGACTTAGCACACGA
TTTGGATTGTTTAAAAATGAATTATCCTGATCATATGCTGAGGTAGATGCTTCTTGAGTAACTTAGTTATGCTTAAATCATGCTTGA
CAGAATCATTCTTGACAACCATGTCTTGTACTTATCTTCTTTATCAGAACTTTTAGGATTTGTCATGATGATGAACAAAACAATTAT
TCTGTTTTTTCCCTTTTTTTCATATACAAGGTTACAATGTGTTTTTATGAATTCACTTTTTGAACTATAAAGTTGTTATACTGCTCAG
TTTTTCTTGTTATCCAAAATAAGTTTCCAGACTTTAAAGATCCAGACCATTCGATCGCTTAGATTTATATTATATTGTGAATTATGC
CAGGTTGATTGAATTTTCTATTTGAATTGACAGTTTCTTATATGAGAGCAGATCTGTGTAATTATTAGAACTCATCTTTTGCCTATG
CTTTTGTGTTACATTGCAGAGACACTGCAAAGTACCGTCCGTTGATGAAAACCAACTTGGTCAGCCATTCACTCAAGTATCTCAC
CCGAACATATCTTGGGTAAGTTAATTTTTTCCTCAATCCTTTTTCAGTCGTAAATTGCTTCTTCCATTTTCAAGTTTTCCTTCTGTTT
TTAAAATCAAATTGGCATGATCTAATCTGTTCCTCCTGCATGTACCAATTGTACATTGACCGTCATTTCAATTTGCTGAGGATCAT
AATAGATTCTATGAATCTTCTTTCTATAATAAAATAAAATGAAATTGGTGAGCAAAACTTTCTGATCTTGAAAGAATTTCAAAACCA
GGTTAAAAAGCAATTTGGCTACATTTCAGTCAAATGCATTTTGTTTTAAATTTCTGTTTGAAAATTAAAAATTAAAAACTCATAATC
ATCTTAAACAAGCCCAAAGTTATTGCATGTGTGAGTGTGTCCTGTAAGTTGGGACAGTTCTCCGGACGGTTAAGAGTATTAGTG
CAATATAAAGAAATCTGCTATATTTTTTCTTTAGTCTTTACCTTGATCTCCTACTTCTTTGTAAACATCATCTGGTAAAAAACACATC
AATTCTATTTTTCCACATTCTGGCTAAAGCAGCAAATCTATCACAAAAGGCTGCATCCATACTTTTTTGTTTTACTTTTAAATTGCC
ATAGTCACTGCTTTCATAATATAACCGCTAGCTACTATATTAATTGCAGATCCCGTAATGTTTAATCTCATCCAGTATTATTGTTCT
TTGGTTTACAGTTATGATATCCAATCCGGCACTCATGACCCTTATGAAGATTGTATTTCTGTCATGAGACTATACAAGAGAATACG
ATCTCAACTTCATCCGGAGGAAGACCATGGAACAATGACCCTGAGTAACAACATTGTTGGCATGCCTGATAGCTGGATATCTAG
GGAACTTGACAACCTCACACCGGATGAACTTTATGCCATGTCTAGATCAGATTATAAGTGTTGGTGCTTGGATTTGATACCAAGA
TTGTCAGCCTGAATTATTGATCTTTGCATTTGTCCATAACATATCATAAGCATATTTCTTCAAGAACTATGAGAATGGAGCATTTG
CAATGATATTTTCTGTGCATATCTTCCTATGCATCGAGTTATCTTCCAGACAAGAAAATTACGAGAAAAGATAGGTGCTTTATGAT
GGAGCTTTTGTAAATATTTAGAACGCTTTGTTCAAATCACATGCCCTTTTTTAGAATGAAGACTTACCTACCTTCTCACTATAATAT
AGGCTCTAACTACCGACACAAAACATAAATGGATGCAAGGCTTGTGT
SEQIDNO: 179
MDAEADPPQNPITRHKCLACYKQYKKKEHLIEHMKTSYHSVHQPRCGVCQKHCKSFESLREHLTGPLPRGICSKIFSQQGCQLCLAL
FDSPGSLIGHRETCRLSAPTCPGTSALPYIDSQFDCQDSSDENHAGEGPGGAVAIDCEMVGGGSDGSLELCARVCLVDEDERLIFHT
YVQPEIPVTNYRYDITGLTEEHLKNAIPLKKVREKLLQILQNGESIGKVRLDGGKARLLVGHDLAHDLDCLKMNYPDHMLRDTAKYRPL
MKTNLVSHSLKYLTRTYLGYDIQSGTHDPYEDCISVMRLYKRIRSQLHPEEDHGTMTLSNNIVGMPDSWISRELDNLTPDELYAMSRS
DYKCWCLDLIPRLSA
SEQIDNO: 180
AAAAAGTAATAGTTATAATTTTAGAAAAATTAATTTTAAGAGAACAAGATATGCTGAATTGGAGCATTTACCTTAAAAAATTTCTCG
GTGGCGTTTTGACTTGAAACAGTCGCTGCTGCGTCTTCTTCTTCTTCATCCGTGGCGTTAGGGTTTTGGTCGGAGTAGCCGTTC
CCCATGGACGCCGAAGCTGACCCTCCTCAAAACCCTATCACAAGACACAAATGCTTGGCATGCTATAAGCAATATAAGAAGAAA
GAGCATCTTATTGAGCACATGAAAACTTCGTATCATTCTGTTCATCAGCCAAGATGCGGGGTCTGTCAAAAGCACTGCAAATCTT
TTGAGTCTCTGAGGGAACATCTTACTGGTCCTTTGCCAAGAGGAATTTGTTCAAAGATTTTCTCTCAACAGGGCTGTCAACTTTG
TCTGGCACTATTTGATAGTCCCGGGTCTCTCATTGGTCATAGAGAAACATGTCGCTTATCTGCCCCTACTTGTCCAGGAACAAGT
GCATTGCCCTATATAGATTCCCAGTTTGATTGTCAAGATTCTTCTGATGAAAACCATGCTGGGGAGGGCCCTGGAGGAGCAGTT
GCAATAGACTGTGAAATGGTTGGTGGTGGAAGTGATGGTTCTCTGGAACTTTGTGCTAGAGTGTGTTTGGTTGATGAAGATGAG
AGATTAATCTTTCATACATATGTACAGCCTGAAATACCTGTTACTAATTACAGATATGATATAACTGGATTGACAGAAGAGCATCT
CAAAAACGCCATTCCACTTAAGAAAGTTCGAGAAAAGCTACTGCAAATTCTACAGAATGGGGAATCCATTGGCAAAGTTAGACT
GGATGGTGGAAAGGCCAGGCTTCTTGTGGGGCATGACTTAGCACACGATTTAGATTGCTTAAAAATGAATTATCCTGATCATAT
GCTGAGAGACACTGCAAAGTATCGTCCTTTGATGAAAACAAATTTGGTCAGCCATTCGCTCAAGTATCTCACCCGAACATATCTT
GGTTATGATATCCAATCCGGCACTCATGACCCTTATGAAGATTGTATTTCTGTCATGAGACTATACAAGAGAATACGATCTCAAC
TTCATCCGGAGGAAGACCATGGAACAATGACTCTGAGTAACAACATTGTTGGCATGCCTGATAGCTGGATATCTAGGGAACTTG
ACAACCTCACACCAGATGAACTTTATGCCATGTCAAGATCAGATTATAAGTGTTGGTGCTTGGATTTGATACCAAGATTGTCAGC
CTGAATTATTGATCTTTGCATTTGTCCATAACATATCATAAGCATATTTCTTCAAGAACCATGGGAATGGAGCATTTGCAATGATA
TTCTCTGTGCATATCTTTCTATGCATCGAGTTATCTTCCGGACAAGAAAATTATGAGAAAAGATAGGGGTGCTTAATGATGGAGC
TTTTGTAAATATTTAGAACGCTTTGTTCAAATCACATGCCCTTGTTTAGAATGAAGACTTACCTACCTTCTCATTATAATATAGGCT
CGAACTACCGACAAAAAAACATAAATGGATGCAAGGCTTGTTCTTGATCCAATTTAAACGGAGCCACTTAGATCTAAATTGCTGA
TTGGTGGTACAAGGAGCTTTCGTTGTCAAAAAAATTGATTTCAAATACAAAATGTGTTAAGTAATTATTTAATTTTCTT
SEQIDNO: 181
CTAACCTGCAAACACCCCTGGCAGTACTTATCATAGGCGGTTTAGGTAGTTGGTTATGTAGAGTGGGTGACTATTATAAATGCTT
GGTGCCGTGGATTAAAAAAAAAAAAAAGTAATAGTTATAATTTTAGAAAAATTAATTTTAAGAGAACAAGATATGCTGAATTGGAG
CATTTACCTTAAAAAATTTCTCGGTGGCGTTTTGACTTGAAACAGTCGCTGCTGCGTCTTCTTCTTCTTCATCCGTGGCGTTAGG
GTTTTGGTCGGAGTAGCCGTTCCCCATGGACGCCGAAGCTGACCCTCCTCAAAACCCTATCACAAGGTCAATTTTCTGCTCCTC
AATTCTCCCTCACTTTAATTTTTTATATTTTATTTCATTTTCCATTATGATCTCCGTGTTGCTTCTGGAGGTAGCCGTTGATCGGAA
ATTCAAACATTGTCATAAGTAAATTAGAAAAATGTTAGAATCAAACTCACTGAATTTTTAAATTGGTTATTACTGCATTTTGTTCTCT
ACTGCACTCTAGAAGAGTCACTTATGTGAAAGAATATTCTAGTTACTTTTCAGTCCGTTTGTGGGACTAATGAGTATCTGCTATAT
ATAATTGCCAGAATTGGCTATATATAATGAATGATGGAATGAAATGTTCCGTGGTTCTGAGTAGATTTAAATATATTGAAATCAGT
GCTCTCTTTTGGGTATAATTGTTTATTTGCTTAGTTTCTTTGAAGAATAACTTATTTCTCCCTACAGACACAAATGCTTGGCATGCT
ATAAGCAATATAAGAAGAAAGAGCATCTTATTGAGCACATGAAAACTTCGTATCATTCTGTTCATCAGCCAAGATGCGGGGTCTG
TCAAAAGCACTGCAAATCTTTTGAGTCTCTGAGGGAACATCTTACTGGTTAGTCATTTCTGTACTCAATCTCTCATGTCAACAGTA
TTTATTGGTTTCATGTTTTAGCTCATTATTTATCCTTTTTTTTTCCTCTATGTTTTAGGTCCTTTGCCAAGAGGAATTTGTTCAAAGA
TTTTCTCTCAACAGGGCTGTCAACTTTGTCTGGCACTATTTGATAGTCCCGGGTCTCTCATTGGTCATAGAGAAACATGTCGCTT
ATCTGCCCCTACTTGTCCAGTAAGTTGATTATTGGCAACCTAAAGCATTTATATTTATATTTAAAATAAAATGTCTTATGTGTACTA
TTCACCCTAAGTAATCAAAGAAACCTGCTCACAATCAGTGTTTCTTGTCTACTCTTTATGTTCCTATTAGGAAGTTTATGTTGTGTA
TGGAACTGTTGCAGGGAACAAGTGCATTGCCCTATATAGATTCCCAGTTTGATTGTCAAGATTCTTCTGATGAAAACCATGCTGG
GGAGGGCCCTGGAGGAGCAGTTGCAATAGACTGTGAAATGGTTGGTGGTGGAAGTGATGGTTCTCTGGAACTTTGTGCTAGAG
TGTGTTTGGTTGATGAAGATGAGAGATTAATCTTTCATACATATGTACAGCCTGAAATACCTGTTACTAATTACAGGTACTTTTTTT
TTTCTATTTCTCTTTTATGTCTATCCTTCCTATTTCTATACTCTCTOTTTCACTTCTAATCCACACAAAGCATTATTGTCAAAGAGAA
AAGTGGACAGGGAAAAGAGAGTGTTTGGGTTGATTTTGTTTGATATTTTCAGATATGATATAACTGGATTGACAGAAGAGCATCT
CAAAAACGCCATTCCACTTAAGAAAGTTCGAGAAAAGCTACTGCAAATTCTACAGAATGGGGAATCCATTGGCAAAGTTAGACT
GGATGGTGGAAAGGCCAGGCTTCTTGTGGGGCATGACTTAGCACACGATTTAGATTGCTTAAAAATGAATTATCCTGATCATAT
GCTGAGGTAGATGCTTCTTGAATAACTTAGTTATGCTAAATCAAGCTTGACAGAATCATTCTTGACAACCATGTCTTGCACTTATT
TTTTTTATCAGAACTTTTAGGATTTGTCATGATGATGAACAAAACAATTATTATGTTTTTTTTCCCTTTTTTTTTCATATACAAGGTT
ACAATGTGTTTTTATGAATTCACTTTTTAAACTATATAGTTGTTATACTGCTCAGTTTTTTTTGTTATCCAAATTAAGTTTCCGGACT
TTAAAGATCCATACTATTCAATTGCTTAGATTTTTATTATATTGTGAATTATGCCAGGTTGATTGAATTTTCTATTTGAATGGACAG
TTTCTTATTTGAGAGCAGATCTGTGTAAATTATTAGAACTCATCTTTTGCCTATGCTTTTGTGTTGCATTGCAGAGACACTGCAAA
GTATCGTCCTTTGATGAAAACAAATTTGGTCAGCCATTCGCTCAAGTATCTCACCCGAACATATCTTGGGCAAGTTAATTTTTTCC
TCAATCCTTTTTCAGTCATAAATTGCTTCTTCCAATTTCAAGTTTTTCTTCTGTTTTTATAATCAGATTGGCATGATCTAATCGGTTC
CTCCTGTATGTACAATTGTACATTGACCATCATTTCAATTTTCTCAGGATCATAATAGACTCTATGAAACATCTGTCCATGATAAAA
TAAAATGAAATTGGTGAGCAAAACTTTCTGATTTTGAAAGAATTTCAAAACCAAGTTAAAAATCAATTTGGCTCCATTTCAATTAGA
TGCATTTTGTTTCGAAATTTATGTTTGAAAATAAAAAATTAAAAACTCATAATCATCTTAAACAAGCCCAAAGTTATTGCACGTGTG
AATGTGTCATGTAAGTTGGGAAAGATCTCCGGATGGTTAGGAGTATTAGTGTAATATAAAGATATCTGCTTTATTTTTTCTTTAGT
CTTTACCTTGATCTCCTACTTTTTAGTAAACATCATCTGGTATAAACACATTAATTCAGTAGTTTTCCACATTTCTGGCTAGAGCAG
CAAACCTATCACAAATGCTGCATCAATACTTTTTTGTTTAACTTTTAAATTGCGATAGCTACTGCTTTCATAATATAACCTGTAGCT
ACTATATTAATTGCAGATCCCATAATTTTTAATCTCATTTCCAGTATTATTGTTCTTTGGTTTACAGTTATGATATCCAATCCGGCA
CTCATGACCCTTATGAAGATTGTATTTCTGTCATGAGACTATACAAGAGAATACGATCTCAACTTCATCCGGAGGAAGACCATGG
AACAATGACTCTGAGTAACAACATTGTTGGCATGCCTGATAGCTGGATATCTAGGGAACTTGACAACCTCACACCAGATGAACTT
TATGCCATGTCAAGATCAGATTATAAGTGTTGGTGCTTGGATTTGATACCAAGATTGTCAGCCTGAATTATTGATCTTTGCATTTG
TCCATAACATATCATAAGCATATTTCTTCAAGAACCATGGGAATGGAGCATTTGCAATGATATTCTCTGTGCATATCTTTCTATGC
ATCGAGTTATCTTCCGGACAAGAAAATTATGAGAAAAGATAGGGGTGCTTAATGATGGAGCTTTTGTAAATATTTAGAACGCTTT
GTTCAAATCACATGCCCTTGTTTAGAATGAAGACTTACCTACCTTCTCATTATAATATAGGCTCGAACTACCGACAAAAAAACATA
AATGGATGCAAGGCTTGTTCTTGATCCAATTTAAACGGAGCCACTTAGATCTAAATTGCTGATTGGTGGTACAAGGAGCTTTCGT
TGTCAAAAAAATTGATTTCAAATACAAAATGTGTTAAGTAATTATTTAATTTTCTT
SEQIDNO: 182
GAGAACAAGATATGCTGAATTGGAGCATTTACCTTAAAAAATTTCTCGGTGGCGTTTTGACTTGAAACAGTCGCTGCTGCGTCTT
CTTCTTCTTCATCCGTGGCGTTAGGGTTTTGGTCGGAGTAGCCGTTCCCCATGGACGCCGAAGCTGACCCTCCTCAAAACCCTA
TCACAAGACACAAATGCTTGGCATGCTATAAGCAATATAAGAAGAAAGAGCATCTTATTGAGCACATGAAAACTTCGTATCATTC
TGTTCATCAGCCAAGATGCGGGGTCTGTCAAAAGCACTGCAAATCTTTTGAGTCTCTGAGGGAACATCTTACTGGTCCTTTGCC
AAGAGGAATTTGTTCAAAGATTTTCTCTCAACAGGGCTGTCAACTTTGTCTGGCACTATTTGATAGTCCCGGGTCTCTCATTGGT
CATAGAGAAACATGTCGCTTATCTGCCCCTACTTGTCCAGGAACAAGTGCATTGCCCTATATAGATTCCCAGTTTGATTGTCAAG
ATTCTTCTGATGAAAACCATGCTGGGGAGGGCCCTGGAGGAGCAGTTGCAATAGACTGTGAAATGGTTGGTGGTGGAAGTGAT
GGTTCTCTGGAACTTTGTGCTAGAGTGTGTTTGGTTGATGAAGATGAGAGATTAATCTTTCATACATATGTACAGCCTGAAATAC
CTGTTACTAATTACAGATATGATATAACTGGATTGACAGAAGAGCATCTCAAAAACGCCATTCCACTTAAGAAAGTTCGAGAAAA
GCTACTGCAAATTCTACAGAATGGGGAATCCATTGGCAAAGTTAGACTGGATGGTGGAAAGGCCAGGCTTCTTGTGGGGCATG
ACTTAGCACACGATTTAGATTGCTTAAAAATGAATTATCCTGATCATATGCTGAGAGACACTGCAAAGTATCGTCCTTTGATGAAA
ACAAATTTGGTCAGCCATTCGCTCAAGTATCTCACCCGAACATATCTTGGTTATGATATCCAATCCGGCACTCATGACCCTTATG
AAGATTGTATTTCTGTCATGAGACTATACAAGAGAATACGATCTCAACTTCATCCGGAGGAAGACCATGGAACAATGACTCTGAG
TAACAACATTGTTGGCATGCCTGATAGCTGGATATCTAGGGAACTTGACAACCTCACACCAGATGAACTTTATGCCATGTCAAGA
TCAGATTATAAGTGTTGGTGCTTGGATTTGATACCAAGATTGTCAGCCTGAATTATTGATCTTTGCATTTGTCCATAACATATCAT
AAGCATATTTCTTCAAGAACCATGGGAATGGAGCATTTGCAATGATATTCTCTGTGCATATCT
SEQIDNO: 183
MDSRRESSETLRNKCAACFRQYNRMEHLVEHMKVSYHSVHEPRCGVCGKHCRSLESLREHLIGPLPKVECARVFGVRGCSICLNVL
DSSAAVRYHRAACQYSRAAPMPRGGSMTGRAVALACKMVGGGSDGSMDLCARVCLVGEDEHVIFQTYVKPTLPVTNYRYEVTGIR
PEYLRDAMPLKVAQRRIQEILCNGESLWKLRPRSYGRAKVLVGHGLDHDLERLGLEYPAFMIRDTAKYPPLMKTSKLSNSLKYLTQAY
LGYDIHTGIQDPYEDCVATMRLYIRMRSQAHQRDYNSGSGEAQNNYPAWRQRELDRMSPEELLALSASDYYCWCLDY
SEQIDNO: 184
ATCGCCAGCCTCGAGATCGATCTCTCTCAAGGGACCTTGCTTGCCGCCACCCCCACCGCAGTTACAGTTATAGCTAGGGATCT
GAGGACTAGCTGATGGACAGCAGGAGGGAGTCCTCGGAGACCTTGAGGAACAAATGCGCAGCCTGCTTCAGGCAGTACAACA
GGATGGAGCACCTGGTGGAGCACATGAAGGTCTCGTACCACTCGGTGCACGAGCCCAGGTGCGGCGTCTGCGGGAAGCACT
GCCGCTCCTTGGAGTCGCTCAGGGAGCATCTCATCGGGCCGTTGCCCAAGGTGGAGTGCGCGCGGGTCTTCGGCGTCCGCG
GCTGCAGCATCTGCCTCAACGTTCTCGACAGCAGCGCCGCCGTCAGATACCACCGTGCGGCCTGCCAGTACTCTCGTGCTGCT
CCGATGCCCAGGGGCGGTAGCATGACTGGGCGCGCGGTCGCCCTGGCTTGCAAGATGGTAGGGGGAGGAAGCGACGGCTC
CATGGACCTTTGTGCGAGGGTGTGCCTCGTTGGAGAAGATGAGCACGTCATCTTCCAGACCTATGTCAAACCTACACTCCCTGT
CACGAACTACAGGTATGAAGTGACTGGGATAAGGCCAGAGTACCTGAGGGACGCAATGCCGCTCAAGGTTGCGCAGAGAAGA
ATCCAGGAAATCCTGTGCAACGGGGAGTCACTGTGGAAGTTACGCCCAAGAAGCTATGGTAGGGCAAAGGTACTCGTTGGTCA
TGGCCTCGACCATGACCTTGAGCGCCTAGGGTTAGAGTACCCGGCATTCATGATCAGGGATACTGCAAAATACCCACCACTAAT
GAAGACTAGCAAGCTGAGTAACTCCCTCAAGTACCTTACACAAGCATACCTCGGGTATGACATCCATACTGGCATTCAGGACCC
CTACGAGGACTGCGTCGCAACAATGAGGCTGTACATCAGGATGAGATCACAGGCTCACCAGAGAGATTACAACTCCGGCTCTG
GCGAGGCCCAGAACAACTATCCAGCCTGGAGGCAGAGGGAGCTCGACAGGATGAGCCCAGAAGAACTCCTGGCACTTTCAGC
ATCAGACTACTACTGCTGGTGCCTGGATTACTAAACCGATCGGCTTATAAGGAAAATAAGGCAGGCGAACGTTGTCGGTTGACA
TGTTCCGTCTATGGCGATCATCTTTAGATGTACAAGTAGCTTGCGGCTTTGCTATATTGGGGGTTAATTAAAGCGTATAAACAAA
GGATTCTATATAAATGTATTGATGCCTTAGATCTGTATAAGAAGCAATATCATGATTTATGATGCATGTGGAAAGAGGATTTTCCT
GCT
SEQIDNO: 185
ATCGCCAGCCTCGAGATCGATCTCTCTCAAGGGACCTTGCTTGCCGCCACCCCCACCGCAGTTACAGTTATAGCTAGGGATCT
GAGGACTAGCTGATGGACAGCAGGAGGGAGTCCTCGGAGACCTTGAGGTAAGAGAGATGCTATTCTTTCAGCTGTCATTGATA
TGCGTACTGCAAATGGCCGTGTGCAGGACTGATTAAGCGCATGCCATATACTGCACCTGTCCCTTGTGGGGGATATATATACAT
ATTAAACACTACTCCTATTATTATTCTAACAAGACATCAAGTCAGTTCACTGATCTATGTAATTAATAAGGCTTGACACATGCATG
GCCATTTCTTGTAACCATGATCCTGTAAAGAGACTGTGGTTGCGAGGAGGCCGGGCTAGCTGTTCATGCATGAAACTGCTCGTG
CAAGGATCAGCTTGGCGCCCTCTCGCATTGGATAGATCCTACATATGTTCCTGAAGGCCAGAATCCATACATACATGATGAATG
ATCTCATACATGTCATGTCCATGACCATCAGGAACAAATGCGCAGCCTGCTTCAGGCAGTACAACAGGATGGAGCACCTGGTG
GAGCACATGAAGGTCTCGTACCACTCGGTGCACGAGCCCAGGTGCGGCGTCTGCGGGAAGCACTGCCGCTCCTTGGAGTCGC
TCAGGGAGCATCTCATCGGTACGTACGTACTGCAGAAACAAAGCTCTCTGCCCCGAAGAAACCAAAAGACGTACGTACAGGCG
TGGTTTCGATCAGTTCTGACGCTTCTTCGATTCTTCCTGTTCTCCACCGTACCGTGTCTCCAGGGCCGTTGCCCAAGGTGGAGT
GCGCGCGGGTCTTCGGCGTCCGCGGCTGCAGCATCTGCCTCAACGTTCTCGACAGCAGCGCCGCCGTCAGATACCACCGTGC
GGCCTGCCAGTACTCTCGTGCTGCTCCGGTACGATGCGTGGTCACCTGATATCACTTGAGAGCTCGATCGGATGCATTGCCTT
CCATCGCCTTTTGCCTGTGGCTTTTGGTCAGTAGTACTAGCTAGATTCTGATATTGTTTTCCACTCCACCATCGTGGTCCCAGAT
GCCCAGGGGCGGTAGCATGACTGGGCGCGCGGTCGCCCTGGCTTGCAAGATGGTAGGGGGAGGAAGCGACGGCTCCATGG
ACCTTTGTGCGAGGGTGTGCCTCGTTGGAGAAGATGAGCACGTCATCTTCCAGACCTATGTCAAACCTACACTCCCTGTCACGA
ACTACAGGTAATCCTCCTACTTCTCGTGCAGTTGCCATCATGTCCGTTACTAAGTTGGCCATCTTTCAGCGAGTACTATACCATA
TGGAGTATATGATAGACGTTGCCGGATCCTATTAAAATAAAGATAAAGAGTAGTTACAGCTGGGAGTAGAAAGAAATTTCGTTCC
ATGTCCTTGAAAGTTGAAGCTGACCGGAAGATGACTATGTGTGTAACATGTTTTCAGGTATGAAGTGACTGGGATAAGGCCAGA
GTACCTGAGGGACGCAATGCCGCTCAAGGTTGCGCAGAGAAGAATCCAGGAAATCCTGTGCAACGGGGAGTCACTGTGGAAG
TTACGCCCAAGAAGCTATGGTAGGGCAAAGGTACTCGTTGGTCATGGCCTCGACCATGACCTTGAGCGCCTAGGGTTAGAGTA
CCCGGCATTCATGATCAGGTGAGGAGCAGGAAAAAAAAACGCAGTTAATTACCAGTTACCACAGTGGTTTTGCCTTTTTGACAG
AAATTGTTGTGCATATGCAGGGATACTGCAAAATACCCACCACTAATGAAGACTAGCAAGCTGAGTAACTCCCTCAAGTACCTTA
CACAAGCATACCTCGGGTATGTCAACTTCTTGGGTGGTCTATATATACAATCAGAACCATCATCGTACTGTTATCTAATATATGTG
AAATAATGCTGACGAGATTCTGTCTTCTTGTAGGTATGACATCCATACTGGCATTCAGGACCCCTACGAGGACTGCGTCGCAAC
AATGAGGCTGTACATCAGGATGAGATCACAGGCTCACCAGAGAGATTACAACTCCGGCTCTGGCGAGGCCCAGAACAACTATC
CAGCCTGGAGGCAGAGGGAGCTCGACAGGATGAGCCCAGAAGAACTCCTGGCACTTTCAGCATCAGACTACTACTGCTGGTG
CCTGGATTACTAAACCGATCGGCTTATAAGGAAAATAAGGCAGGCGAACGTTGTCGGTTGACATGTTCCGTCTATGGCGATCAT
CTTTAGATGTACAAGTAGCTTGCGGCTTTGCTATATTGGGGGTTAATTAAAGCGTATAAACAAAGGATTCTATATAAATGTATTGA
TGCCTTAGATCTGTATAAGAAGCAATATCATGATTTATGATGCATGTGGAAAGAGGATTTTCCTGCT
SEQIDNO: 186
MDSSSDAHGRHRCAACFRQFNKMEHLVEHMRAARHSGHEPRCDICRKHCRSFEALRDHLGVGGSTLPKAASCADAFAARGCAICL
RVLAGAGAASLGAHRAACRLSRTPPPRALQQHHRTQPQGGALALGCKMVGAGSDGSLDVCARVCVIDEQENVLFEAFVRPLLPVTH
YRYETTGIRPEHLRDGASVTVKSAQRRVEELLLDGEQPWRARTSRGRARLLVGHGLDHDLHALHMDYPAYLKRDTATYPPLMKTSK
LSNSLRFLTLNYLGYEIQTGHQHPFEDCVAAMRLYRRMRGQQHHPRADAHAPAPAADDQQPFPSWRQRELERMTPEDLLRLSTPD
YHCWCLDA
SEQIDNO: 187
ATCGGTCGACCAGATCAACCGAGCAGTTGGGGTGAACGAACCGTGGATCGACGTCGTACTCGTACGTACGTACGTACTGTACG
TGACGATGGATAGCTCCTCGGACGCTCACGGGCGTCACAGGTGCGCGGCGTGCTTCCGGCAGTTCAACAAGATGGAGCACCT
GGTGGAGCACATGCGGGCGGCGCGGCACTCGGGGCACGAGCCCCGCTGCGACATCTGCCGCAAGCACTGCCGCTCCTTCGA
GGCGCTCAGGGACCACCTCGGCGTCGGCGGCTCCACGCTGCCCAAGGCCGCCAGCTGCGCCGACGCCTTCGCCGCGCGGG
GCTGCGCCATCTGCCTCCGCGTCCTCGCCGGCGCCGGCGCCGCGTCGCTCGGAGCCCACCGCGCGGCGTGCCGGCTCTCG
CGCACCCCGCCGCCGAGGGCGCTGCAGCAGCATCACCGGACGCAGCCGCAAGGAGGCGCGCTCGCGCTGGGCTGCAAGAT
GGTCGGCGCCGGCAGCGACGGATCCCTGGACGTGTGCGCGCGGGTGTGCGTCATCGACGAGCAGGAGAACGTCCTGTTCGA
GGCCTTCGTGAGGCCGCTCCTTCCCGTGACGCACTACCGGTACGAGACGACGGGGATCCGGCCGGAGCACCTCCGCGACGG
CGCGAGCGTGACGGTGAAGAGCGCGCAGCGCCGGGTGGAGGAGCTGCTGCTCGACGGCGAGCAGCCGTGGAGGGCGCGCA
CCAGCCGGGGCAGGGCGCGCCTGCTGGTCGGCCACGGCCTCGACCACGACCTCCACGCGCTGCACATGGACTACCCGGCCT
ACCTCAAGCGCGACACGGCCACGTACCCGCCGCTCATGAAGACCAGCAAGCTCAGCAACTCGCTCCGCTTCCTCACGCTCAA
CTACCTCGGCTACGAGATCCAGACGGGGCACCAGCACCCCTTCGAGGACTGCGTCGCCGCCATGCGCCTCTACCGCAGGATG
CGCGGGCAGCAGCACCACCCCAGGGCCGACGCACACGCACCGGCGCCGGCCGCGGACGACCAGCAGCCGTTCCCGTCGTG
GAGGCAGCGGGAGCTGGAGCGCATGACGCCCGAGGACCTCCTCCGGCTCTCCACGCCGGACTACCACTGCTGGTGCCTOGA
TGCGTAGCGTGTGGCTTGAGCATCTATCTCCGTCAGAGGCTCAGAGCCAGCCCGAGCGCATCCTGATCGCGTTCCCGCCCCAT
GACCAATGACGTGGTCGTGTATAAGATTCTGCACCGTTCAGCCGTTCGTCAGCTCCTCCGGCTCACACGAACTTCTGTACCAAG
TATCTAATCTATCCATAGCTACTCCAAAAGTACAATCCGATCCACCCGTATGATTGAGGGAGAGGGACAGAGAATGTACCTGTG
GATCCGTTCGATATAAAACGCCTGTTAGAGTATATA
SEQIDNO: 188
ATCGGTCGACCAGATCAACCGAGCAGTTGGGGTGAACGAACCGTGGATCGACGTCGTACTCGTACGTACGTACGTACTGTACG
TGACGATGGATAGCTCCTCGGACGCTCACGGGTGAGCTTTCTTTCACAAACTATATGCATGCTTTGAGGTAAACTCTACCGTGC
TTCGGACACCTGGGTTTTGTTTTGTGGACCATTTCGATCACTAAAATCTAGCTCCATGCATCTGTGTCTGTTCGAAGTCGTTTTTT
CTTGTTTCAATGGCAATGCCTACTATGATTTTACTAGAACCATTGTGGACATTATTATOTTCACTCCTAACAGCGCCCTGCCAAG
GACATATGCTACTGCTACTCTACCAGTCAAAGGCACTTTTTTCTATGGCCGGCCTGGCTGTGAAATCTATGGCCGGTCATTTTTT
CTTCTTCGTGTTAGAACAAAATCCAAATAGACTTGTATTTAAAGACCAGCACTTTGAACTTGTGTTAGGAATCTTGTATAAAAAAA
AGAAAAGTAAAAATACGAGCGGCTCCCCGTCTACTCGTAACGTACAGTGTAACGAGGAATGACTTGTTTGGCAGGCGTCACAG
GTGCGCGGCGTGCTTCCGGCAGTTCAACAAGATGGAGCACCTGGTGGAGCACATGCGGGCGGCGCGGCACTCGGGGCACGA
GCCCCGCTGCGACATCTGCCGCAAGCACTGCCGCTCCTTCGAGGCGCTCAGGGACCACCTCGGCGTCGGCGGCTCCACGCT
GCCCAAGGCCGCCAGCTGCGCCGACGCCTTCGCCGCGCGGGGCTGCGCCATCTGCCTCCGCGTCCTCGCCGGCGCCGGCG
CCGCGTCGCTCGGAGCCCACCGCGCGGCGTGCCGGCTCTCGCGCACCCCGCCGCCGAGGGCGCTGCAGCAGCATCACCGG
ACGCAGCCGCAAGGAGGCGCGCTCGCGCTGGGCTGCAAGATGGTCGGCGCCGGCAGCGACGGATCCCTGGACGTGTGCGC
GCGGGTGTGCGTCATCGACGAGCAGGAGAACGTCCTGTTCGAGGCCTTCGTGAGGCCGCTCCTTCCCGTGACGCACTACCGG
TACGAGACGACGGGGATCCGGCCGGAGCACCTCCGCGACGGCGCGAGCGTGACGGTGAAGAGCGCGCAGCGCCGGGTGGA
GGAGCTGCTGCTCGACGGCGAGCAGCCGTGGAGGGCGCGCACCAGCCGGGGCAGGGCGCGCCTGCTGGTCGGCCACGGC
CTCGACCACGACCTCCACGCGCTGCACATGGACTACCCGGCCTACCTCAAGCGCGACACGGCCACGTACCCGCCGCTCATGA
AGACCAGCAAGCTCAGCAACTCGCTCCGCTTCCTCACGCTCAACTACCTCGGCTACGAGATCCAGACGGGGCACCAGCACCC
CTTCGAGGACTGCGTCGCCGCCATGCGCCTCTACCGCAGGATGCGCGGGCAGCAGCACCACCCCAGGGCCGACGCACACGC
ACCGGCGCCGGCCGCGGACGACCAGCAGCCGTTCCCGTCGTGGAGGCAGCGGGAGCTGGAGCGCATGACGCCCGAGGACC
TCCTCCGGCTCTCCACGCCGGACTACCACTGCTGGTGCCTCGATGCGTAGCGTGTGGCTTGAGCATCTATCTCCGTCAGAGGC
TCAGAGCCAGCCCGAGCGCATCCTGATCGCGTTCCCGCCCCATGACCAATGACGTGGTCGTGTATAAGATTCTGCACCGTTCA
GCCGTTCGTCAGCTCCTCCGGCTCACACGAACTTCTGTACCAAGTATCTAATCTATCCATAGCTACTCCAAAAGTACAATCCGAT
CCACCCGTATGATTGAGGGAGAGGGACAGAGAATGTACCTGTGGATCCGTTCGATATAAAACGCCTGTTAGAGTATATA
SEQIDNO: 189
MDSRRETSETLRNKCAACFRQYNKMEHLVEHMKVSYHSVHEPRCGACGKHCRSFESLREHLIGPLPKVECARVFAARGCGICLNIF
DSPATVRYHRPACQYSRAAPMPKAGSARGRAVAMACKMVGGGSDGSLDLCARLCIIGEDETVIFQTYVKPTAPVTNYRYEVTGIRPE
YLRDAMPLKLAQRRVQDILCNGEPLWKIRPRSYGRARVLVGHGVDQDLERLGLEYPAFMIRDTAKYPPLMKTSKLSNPLKYLTQAYL
GYDVHTGVQDPYEDCVAAMRLYIRMRSQAHPRDYASGSGEVQNNYPAWRQRELERMSPEELLALSGSDYYCWCLDP
SEQIDNO: 190
CGCGGTTTCAGAGTTCAGAGAGCTTGATCGATCGATCTATCCATAGGAATTTCAGGAGCGATGGACAGCAGGAGGGAGACCTC
GGAGACCTTGAGGAACAAGTGTGCGGCGTGCTTCAGGCAGTACAACAAGATGGAACATCTGGTGGAGCACATGAAGGTGTCG
TATCACTCCGTCCACGAGCCCAGGTGCGGCGCCTGCGGGAAGCACTGCCGCTCCTTCGAGTCCCTCAGGGAGCACCTCATCG
GGCCTTTGCCCAAGGTGGAGTGCGCGCGCGTCTTCGCCGCCCGGGGCTGCGGCATCTGCCTCAACATCTTCGACAGCCCGG
CCACCGTCAGATATCACCGCCCCGCCTGCCAGTACTCCCGTGCGGCTCCGATGCCTAAGGCTGGCTCAGCACGAGGACGCGC
GGTCGCCATGGCCTGCAAGATGGTCGGAGGAGGAAGCGACGGCTCGCTGGACCTCTGCGCTAGACTGTGCATCATTGGGGAA
GACGAGACCGTCATCTTCCAGACCTACGTGAAACCCACGGCGCCTGTCACCAACTACAGGTATGAGGTGACTGGAATAAGGCC
GGAGTACCTGAGGGACGCAATGCCACTGAAGCTTGCCCAGAGGAGGGTCCAGGACATCCTGTGCAACGGGGAGCCGCTGTG
GAAGATCCGGCCGAGGAGCTATGGAAGGGCAAGGGTCCTCGTTGGACATGGCGTGGACCAGGACCTTGAGCGCCTAGGGTT
GGAGTACCCAGCATTCATGATCAGGGACACTGCAAAGTATCCACCACTGATGAAAACCAGCAAGCTGAGCAATCCCCTAAAGTA
CCTTACACAAGCATATCTTGGGTATGATGTGCACACTGGCGTTCAGGATCCGTACGAGGACTGCGTGGCAGCGATGAGACTAT
ATATCAGGATGAGATCGCAAGCTCACCCGAGAGACTATGCCTCCGGTTCAGGGGAGGTGCAGAATAACTACCCGGCCTGGAG
GCAGAGGGAGCTGGAGAGGATGAGCCCAGAAGAACTGCTGGCACTTTCAGGATCAGATTACTACTGCTGGTGCCTGGACCCC
TAAACTGATGAGCTGAAGAGAACAAGGCAGGACCAATGGTGCTGGTTTTCTCATATTCCATCCATAATAATAATGATTACGTGTG
TCTAATTATCTTTGGACTTATGTATGGTGGGATTGAGTATCATATCGGTGATCTAGAGTTGTATGAGTCTTTATTTAATAGCAACC
ATATATAGCAAGAGGATTTCTACTTTCCTTTATGCATATTGAAAACCACAAAATCAGTAGTTAGCCAAAAAAATGACAAAATCAGT
GCAACAATTATACAACACACGGGCAAATAATCAGGCCTATAACATCATATTTAATGAGTTTACACTCTGTAGTCTGTACATGTTGT
GAGACTATGTCAGTACTAAGGATACATTATGCAGCAGAATATCGTACCTGTGCAAATCAGCGATAATCGAGTGTGTGCACAACT
GTCGAAAT
SEQIDNO: 191
CGCGGTTTCAGAGTTCAGAGAGCTTGATCGATCGATCTATCCATAGGAATTTCAGGAGCGATGGACAGCAGGAGGGAGACCTC
GGAGACCTTGAGGTAAGCATGCGCAAAGCTGATAAGAAATGACTGTCATTCTTGTATTGTGCTAATTTTGTGTGTCCCATGATAA
AGAGAGATCTACTCATTAATCTAGCTTGTAAACCTTGAAACTCAAACCTAATACTCCGTATGAGATTTCTTTATTTTTTCCCTTCCA
AACTAGCTGTCTCCGAGCTAGCTACACAAGGCTACATTTTTTTTTAATCTCTGCGAAGTGCTAAATAAACGGCCAGAACTGGCAC
CCATTTCTACTGCATTTGACCGATCGATGCTTGCCTTGTCCTGTAATTACATGTTAAATATCCTAATACGTGTGTGTGTGCATGTG
TCCCCATGGATGGATATATCAGGAACAAGTGTGCGGCGTGCTTCAGGCAGTACAACAAGATGGAACATCTGGTGGAGCACATG
AAGGTGTCGTATCACTCCGTCCACGAGCCCAGGTGCGGCGCCTGCGGGAAGCACTGCCGCTCCTTCGAGTCCCTCAGGGAGC
ACCTCATCGGTAAGCAAACAATTATCCACAAATGGATTAAGATTTTGTTAGACACATACTGCAAATTCTGATCTGTGCGTGTCTTG
ATGAATGAAGGGCCTTTGCCCAAGGTGGAGTGCGCGCGCGTCTTCGCCGCCCGGGGCTGCGGCATCTGCCTCAACATCTTCG
ACAGCCCGGCCACCGTCAGATATCACCGCCCCGCCTGCCAGTACTCCCGTGCGGCTCCGGTAATGCCTCCGTCGTTGTCGTC
CATGAACTCTATATATTCAGTTTGCACGGAGGCTGATGCTTCTTTTCCTCCCCCTCTATTCTGTCAGATGCCTAAGGCTGGCTCA
GCACGAGGACGCGCGGTCGCCATGGCCTGCAAGATGGTCGGAGGAGGAAGCGACGGCTCGCTGGACCTCTGCGCTAGACTG
TGCATCATTGGGGAAGACGAGACCGTCATCTTCCAGACCTACGTGAAACCCACGGCGCCTGTCACCAACTACAGGTATAGCCT
GATCGACATTGCCCTGGAATCCATCACTGGTTCTGCTTAATGGGCTGTTTGCTGAAATGATTTTCCTCGGTTCAGGTATGAGGT
GACTGGAATAAGGCCGGAGTACCTGAGGGACGCAATGCCACTGAAGCTTGCCCAGAGGAGGGTCCAGGACATCCTGTGCAAC
GGGGAGCCGCTGTGGAAGATCCGGCCGAGGAGCTATGGAAGGGCAAGGGTCCTCGTTGGACATGGCGTGGACCAGGACCTT
GAGCGCCTAGGGTTGGAGTACCCAGCATTCATGATCAGGTGAGCACCACAGCACAATTTTTTTTTGCTATGGCCCTAATTAAGA
TGTACTGGTAGATCTTTGCCGCTTTGCTTAGTGAGTTATGGTTCAGGGCCTACAGTTTTTTTTTTCAATCTCTGGATATGCAGGG
ACACTGCAAAGTATCCACCACTGATGAAAACCAGCAAGCTGAGCAATCCCCTAAAGTACCTTACACAAGCATATCTTGGGTATG
TCACTCCCTACCCTGGGCTACACAATCTCTCTGCACCTTTGGTCATTTTCTTAATGCTGAAGAGATTGTCCGTCTATCCCATTTG
CAGGTATGATGTGCACACTGGCGTTCAGGATCCGTACGAGGACTGCGTGGCAGCGATGAGACTATATATCAGGATGAGATCGC
AAGCTCACCCGAGAGACTATGCCTCCGGTTCAGGGGAGGTGCAGAATAACTACCCGGCCTGGAGGCAGAGGGAGCTGGAGA
GGATGAGCCCAGAAGAACTGCTGGCACTTTCAGGATCAGATTACTACTGCTGGTGCCTGGACCCCTAAACTGATGAGCTGAAG
AGAACAAGGCAGGACCAATGGTGCTGGTTTTCTCATATTCCATCCATAATAATAATGATTACGTGTGTCTAATTATCTTTGGACTT
ATGTATGGTGGGATTGAGTATCATATCGGTGATCTAGAGTTGTATGAGTCTTTATTTAATAGCAACCATATATAGCAAGAGGATTT
CTACTTTCCTTTATGCATATTGAAAACCACAAAATCAGTAGTTAGCCAAAAAAATGACAAAATCAGTGCAACAATTATACAACACA
CGGGCAAATAATCAGGCCTATAACATCATATTTAATGAGTTTACACTCTGTAGTCTGTACATGTTGTGAGACTATGTCAGTACTAA
GGATACATTATGCAGCAGAATATCGTACCTGTGCAAATCAGCGATAATCGAGTGTGTGCACAACTGTCGAAAT
SEQIDNO: 192
GGTTTCAGAGTTCAGAGAGCTTGATCGATCGATCTATCCATAGGAATTTCAGGAGCGATGGACAGCAGGAGGGAGACCTCGGA
GACCTTGAGGAACAAGTGTGCGGCGTGCTTCAGGCAGTACAACAAGATGGAACATCTGGTGGAGCACATGAAGGTGTCGTATC
ACTCCGTCCACGAGCCCAGGTGCGGCGCCTGCGGGAAGCACTGCCGCTCCTTCGAGTCCCTCAGGGAGCACCTCATCGGGC
CTTTGCCCAAGGTGGAGTGCGCGCGCGTCTTCGCCGCCCGGGGCTGCGGCATCTGCCTCAACATCTTCGACAGCCCGGCCAC
CGTCAGATATCACCGCCCCGCCTGCCAGTACTCCCGTGCGGCTCCGATGCCTAAGGCTGGCTCAGCACGAGGACGCGCGGTC
GCCATGGCCTGCAAGATGGTCGGAGGAGGAAGCGACGGCTCGCTGGACCTCTGCGCTAGACTGTGCATCATTGGGGAAGACG
AGACCGTCATCTTCCAGACCTACGTGAAACCCACGGCGCCTGTCACCAACTACAGGTATGAGGTGACTGGAATAAGGCCGGAG
TACCTGAGGGACGCAATGCCACTGAAGCTTGCCCAGAGGAGGGTCCAGGACATCCTGTGCAACGGGGAGCCGCTGTGGAAGA
TCCGGCCGAGGAGCTATGGAAGGGCAAGGGTCCTCGTTGGACATGGCGTGGACCAGGACCTTGAGCGCCTAGGGTTGGAGT
ACCCAGCATTCATGATCAGGGACACTGCAAAGTATCCACCACTGATGAAAACCAGCAAGCTGAGCAATCCCCTAAAGTACCTTA
CACAAGCATATCTTGGGTATGATGTGCACACTGGCGTTCAGGATCCGTACGAGGACTGCGTGGCAGCGATGAGACTATATATC
AGGATGAGATCGCAAGCTCACCCGAGAGACTATGCCTCCGGTTCAGGGGAGGTGCAGAATAACTACCCGGCCTGGAGGCAGA
GGGAGCTGGAGAGGATGAGCCCAGAAGAACTGCTGGCACTTTCAGGATCAGATTACTACTGCTGGTGCCTGGACCCCTAAACT
GATGAGCTGAAGAGAACAAGGCAGGACCAATGGTGCTGGTTTTCTCATATTCCATCCATAATAATAATGATTACGTGTGTCTAAT
TATCTTTGGACTTATGTATGGTGGGATTGAGTATCATATCGGTGATCTAGA
SEQIDNO: 193
MDSRRESAETLRNKCSACFRQYNKMEHLVEHMKVSYHSVHEPKCGACRKHCRSFESLREHLIGPLPKAECARVFSARGCGICLNIF
DSPAAARYHRQACQYSRAAPMPKGGAGGRAVAMACKMVGGGSDGSVDLCARVCLIGEDENVIFQTYVKPTAPVTNYRYEVTGIRP
EYLRDAMPLKLVQRRIQDILCNGEPLWKIRPRSYGRARILVGHIVDHDLERLGLEYPAFMIRDTAKYPPLMKTSKLSNTLKYLTQAYLG
YDVHTGIQDPYEDCVAAMRLYIRMRSQAHPRDYASGSGEVQNNYPAWRQREMERMSPEELLALSGSDYYCWCLDP
SEQIDNO: 194
TTTTTCTCGTGAAGGTTCCATCTCTGATCTCTCAACGAGGCTGGCCTATAAATAGGCGTTCCCATACTGCTCATCCTCGCCAACC
TCGAGATCTGAGAGATCTTGATCGACCTGCAGGAAGAATCTGTGGAGCTCGATGGACAGCAGGAGGGAGTCCGCGGAGACCC
TGAGGAACAAGTGCTCGGCGTGCTTCCGGCAGTACAACAAGATGGAGCACCTTGTGGAGCACATGAAGGTGTCGTATCACTCG
GTCCACGAGCCCAAGTGCGGCGCCTGCAGGAAGCACTGCCGCTCCTTCGAGTCCCTCAGGGAGCACCTCATCGGTCCGCTGC
CCAAGGCGGAATGCGCGCGCGTCTTCAGCGCCCGGGGCTGCGGCATCTGCCTCAACATCTTCGATAGCCCTGCCGCCGCCAG
ATATCACCGTCAAGCCTGCCAGTACTCCCGCGCTGCTCCGATGCCAAAGGGTGGCGCAGGTGGGCGTGCGGTTGCCATGGCC
TGCAAGATGGTCGGAGGAGGGAGCGACGGCTCTGTGGACCTCTGCGCAAGAGTGTGCCTTATTGGAGAAGATGAGAACGTCA
TCTTCCAGACCTATGTAAAACCTACAGCTCCGGTCACAAACTACAGGTATGAGGTTACTGGGATAAGGCCCGAGTACCTGAGG
GATGCAATGCCACTGAAACTTGTGCAGAGGAGGATCCAGGACATCCTGTGCAACGGGGAGCCGCTGTGGAAGATACGGCCGA
GGAGCTATGGAAGGGCAAGGATCCTTGTTGGGCATATCGTGGACCATGACCTCGAGCGCCTAGGTTTGGAGTACCCAGCATTC
ATGATCAGGGACACCGCAAAGTACCCACCGCTGATGAAAACCAGCAAGCTGAGCAATACCCTGAAGTACCTCACACAAGCATA
TCTTGGGTATGATGTCCATACTGGCATTCAGGATCCATACGAGGACTGCGTCGCGGCGATGAGGCTATATATCAGGATGAGAT
CACAAGCTCACCCGAGAGACTACGCCTCCGGTTCAGGGGAGGTGCAGAATAACTACCCGGCCTGGAGGCAGAGGGAGATGGA
GAGGATGAGCCCAGAAGAACTCCTGGCACTTTCAGGATCAGACTACTACTGCTGGTGCCTGGACCCCTAGACTGATGATGAGC
TAAAGAGAACAAGGCAGGGCTGGCCGACTGATGTTGGTTTGGTCATATTCCATCCGTAGTAATACCGACTACGTATGTCTAATT
ATCTT
SEQIDNO: 195
TTTTTCTCGTGAAGGTTCCATCTCTGATCTCTCAACGAGGCTGGCCTATAAATAGGCGTTCCCATACTGCTCATCCTCGCCAACC
TCGAGATCTGAGAGATCTTGATCGACCTGCAGGAAGAATCTGTGGAGCTCGATGGACAGCAGGAGGGAGTCCGCGGAGACCC
TGAGGTAACAAAACGTTGAGAAACACCATTTTTTCTTGGCTAACTTCGTTGATAACCCCAGTTTAAGTCCACTTTCGATGCTTGC
CTAACTGAAGCATGTCTGTGTGTGTACCAGGAACAAGTGCTCGGCGTGCTTCCGGCAGTACAACAAGATGGAGCACCTTGTGG
AGCACATGAAGGTGTCGTATCACTCGGTCCACGAGCCCAAGTGCGGCGCCTGCAGGAAGCACTGCCGCTCCTTCGAGTCCCT
CAGGGAGCACCTCATCGGTAAGCTACCAAACACATATCGATCTCTAGCTGCAAACATCTCTAGCTATGGCATCAACAGCAGCTG
AACTGAACGCCGCTCCTTGTGATCTTGATCTTGGAAATGAAGGTCCGCTGCCCAAGGCGGAATGCGCGCGCGTCTTCAGCGCC
CGGGGCTGCGGCATCTGCCTCAACATCTTCGATAGCCCTGCCGCCGCCAGATATCACCGTCAAGCCTGCCAGTACTCCCGCG
CTGCTCCGGTAATTCATGTTCTCCATTCCGTTCAATCGCCACCATCGACCTCTTCTTAACATGCATGGATGCTAGAGCTCCCGG
CCAACTTTTCACAGAAATGTTGGCGAGAGAGCAGAAATCAGAATCTGATGTCTCTCTTCTCCCTCACTTCTTTTTTCTCAGATGC
CAAAGGGTGGCGCAGGTGGGCGTGCGGTTGCCATGGCCTGCAAGATGGTCGGAGGAGGGAGCGACGGCTCTGTGGACCTCT
GCGCAAGAGTGTGCCTTATTGGAGAAGATGAGAACGTCATCTTCCAGACCTATGTAAAACCTACAGCTCCGGTCACAAACTACA
GGTAGCTCACCTTGGAATCCATTACCAGTACTGCTTACTAGCTATACAAGGATCAACTAGATGGATTGTTTTCTGAAGTGCAAAA
GATGAAGCTGACCATTTGGCTCTTCAGGTATGAGGTTACTGGGATAAGGCCCGAGTACCTGAGGGATGCAATGCCACTGAAAC
TTGTGCAGAGGAGGATCCAGGACATCCTGTGCAACGGGGAGCCGCTGTGGAAGATACGGCCGAGGAGCTATGGAAGGGCAA
GGATCCTTGTTGGGCATATCGTGGACCATGACCTCGAGCGCCTAGGTTTGGAGTACCCAGCATTCATGATCAGGTACAGATCAT
TGCTCAGTGAGCTATAGTTTGTTCCTGCTATCTCTTCCTTAATTGCCTCGAAATTTTACTATTTTCTTAACATAATTTATCCATGGA
CGTGCAGGGACACCGCAAAGTACCCACCGCTGATGAAAACCAGCAAGCTGAGCAATACCCTGAAGTACCTCACACAAGCATAT
CTTGGGTATGTCACTTGATAACTGAGCTATAAAACAAACACTCTGAACCCTTTGGTCATTGCCTAATCCTCAAGGGATTGCCCTT
CTATCCGGTTGTAGGTATGATGTCCATACTGGCATTCAGGATCCATACGAGGACTGCGTCGCGGCGATGAGGCTATATATCAG
GATGAGATCACAAGCTCACCCGAGAGACTACGCCTCCGGTTCAGGGGAGGTGCAGAATAACTACCCGGCCTGGAGGCAGAGG
GAGATGGAGAGGATGAGCCCAGAAGAACTCCTGGCACTTTCAGGATCAGACTACTACTGCTGGTGCCTGGACCCCTAGACTGA
TGATGAGCTAAAGAGAACAAGGCAGGGCTGGCCGACTGATGTTGGTTTGGTCATATTCCATCCGTAGTAATACCGACTACGTAT
GTCTAATTATCTT
SEQIDNO: 196
TGTGGAGCGCGATGGACAGCAGAAGGGAGTCCGCGGAGACCCTGAGGTAACAAGACGTTGAGAAACGCCATTTTTCTTAACAA
ACTGCAATAGCTCGAAGGAAGGGTGAGATAGACGCTTGGCCTAACTGAAGCATGTCTGTGTGTGTACCAGGAACAAGTGCTCG
GCGTGCTTCCGGCAGTACAACAAGATGGAGCACCTTGTGGAGCACATGAAGGTGTCGTACCACTCGGTCCACGAGCCCAAGT
GCGGCGCCTGCAGGAAGCACTGCCGCTCCTTCGAGTCCCTCAGGGAGCACCTCATAGGTAAGCTACCAGCAAACTCCACGAC
CACCAAATCTCTAGCTGCATGCAAACATCTCTATAGCTATGGCATCAACAGCAGCTGAACTGTGCACGCTGGGCTCATGCATCT
TTTATATTCTCCCGTCCGATCTTTGAAGTATGTCCTGACAGTATATATGTCTTGTTGCCTCTTCATCTTGGCAATGAAGGTCCATT
GCCCAAGGCCGAATGCGCGCGCGTCTTCAGCGCCCGGGGCTGTGGCATCTGCCTCAACATCTTTGACAGCCCCGCCGCCGCC
AGATATCACCGTCACGCCTGCCAATACTCCCGCGCTGCTCCGGTAATTAGTGTTCTCCACTCCGTTCAGTCTCCGCCATGGAGC
TCTTCATAGCTTGGATGCTAGAGCTCCCGGCCAACTTTTTACAAAAATGTTGCTGGGAGAGCAGAAATCAGAACCTGATGTTTCT
CTTCTCCCTCTCTTTTGCGTTCTTTTTCTCAGATGCCAAAGGGTGGCGCAGGTGGACGCGCGGTTGCCATGGCCTGTAAGATG
GTCGGAGGAGGGAGCGACGGCTCCGTGGACCTCTGCGCAAGAGTGTGCCTTATTGGAGAAGATGAGAACGTCATCTTCCAGA
CCTATGTAAAACCTACAGCTCCGGTCACAAACTACAGGTAGCTCACCTTGGAATCCATTACCAGTACTGCTTACTAGCTATACAA
GGATCAACTAGATGGATTGTTTTCTGAAGTGCAAAAGATGAAGCTGACCATTTGGCTCTTCAGGTATGAGGTTACTGGGATAAG
GCCCGAGTACCTGAGGGATGCAATGCCACTGAAACTTGTGCAGAGGAGGATCCAGGACATCCTGTGCAACGGGGAGCCGCTG
TGGAAGATACGGCCGAGGAGCTATGGAAGGGCAAGGATCCTTGTTGGGCATATCGTGGACCATGACCTCGAGCGCCTAGGTT
TGGACTACCCAGCATTCATGATCAGGTACAGATCATTGCTCAGTGAGCTATTGTTCCTACCATATCTTCCTTAATTGCCTCGAAA
TTTTACTATTTTCTTAACACTATTTATCCATGGACATGCAGGGACACCGCAAAGTACCCACCGCTGATGAAAACCAGCAAGCTGA
GCAATACCCTGAAGTACCTCACACAAGCATATCTTGGGTATGTCACTTGATAACTGATCTATAAAACAAACTCTCTGAACCCTTT
GGTCATTGCCTAATGCTCAAGGGATTGCCCCTCTATCCCGTTATAGGTATGATGTCCATACTGGCATTCAGGATCCATACGAGG
ACTGCGTCGCGGCGATGAGACTATATATCAGGATGAGATCACAAGCTCACCCGAGAGACTACGCCTCCGGTTCAGGGGAGGT
GCAGAATAACTACCCGGCCTGGAGGCAGAGGGAGATGGAGAGGATGAGCCCAGAAGAGCTCCTGGCACTTTCAGGATCAGAC
TACTACTGCTGGTGCCTGGACCCCTAGA
SEQIDNO: 197
MDSRRENKCSACFRQYNKMEHLVEHMKVSYHSVHEPKCGACRKHCRSFESLREHLIGPLPKAECARVFSARGCGICLNIFDSPAAA
RYHRHACQYSRAAPMPKGGAGGRAVAMACKMVGGGSDGSVDLCARVCLIGEDENVIFQTYVKPTAPVTNYRYEVTGIRPEYLRDA
MPLKLVQRRIQDILCNGEPLWKIRPRSYGRARILVGHIVDHDLERLGLDYPAFMIRDTAKYPPLMKTSKLSNTLKYLTQAYLGYDVHTGI
QDPYEDCVAAMRLYIRMRSQAHPRDYASGSGEVQNNYPAWRQREMERMSPEELLALSGSDYYCWCLDP
SEQIDNO: 198
TGTGGAGCGCGATGGACAGCAGAAGGGAGAACAAGTGCTCGGCGTGCTTCCGGCAGTACAACAAGATGGAGCACCTTGTGGA
GCACATGAAGGTGTCGTACCACTCGGTCCACGAGCCCAAGTGCGGCGCCTGCAGGAAGCACTGCCGCTCCTTCGAGTCCCTC
AGGGAGCACCTCATAGGTCCATTGCCCAAGGCCGAATGCGCGCGCGTCTTCAGCGCCCGGGGCTGTGGCATCTGCCTCAACA
TCTTTGACAGCCCCGCCGCCGCCAGATATCACCGTCACGCCTGCCAATACTCCCGCGCTGCTCCGATGCCAAAGGGTGGCGC
AGGTGGACGCGCGGTTGCCATGGCCTGTAAGATGGTCGGAGGAGGGAGCGACGGCTCCGTGGACCTCTGCGCAAGAGTGTG
CCTTATTGGAGAAGATGAGAACGTCATCTTCCAGACCTATGTAAAACCTACAGCTCCGGTCACAAACTACAGGTATGAGGTTAC
TGGGATAAGGCCCGAGTACCTGAGGGATGCAATGCCACTGAAACTTGTGCAGAGGAGGATCCAGGACATCCTGTGCAACGGG
GAGCCGCTGTGGAAGATACGGCCGAGGAGCTATGGAAGGGCAAGGATCCTTGTTGGGCATATCGTGGACCATGACCTCGAGC
GCCTAGGTTTGGACTACCCAGCATTCATGATCAGGGACACCGCAAAGTACCCACCGCTGATGAAAACCAGCAAGCTGAGCAAT
ACCCTGAAGTACCTCACACAAGCATATCTTGGGTATGATGTCCATACTGGCATTCAGGATCCATACGAGGACTGCGTCGCGGC
GATGAGACTATATATCAGGATGAGATCACAAGCTCACCCGAGAGACTACGCCTCCGGTTCAGGGGAGGTGCAGAATAACTACC
CGGCCTGGAGGCAGAGGGAGATGGAGAGGATGAGCCCAGAAGAGCTCCTGGCACTTTCAGGATCAGACTACTACTGCTGGTG
CCTGGACCCCTAGA
SEQIDNO: 199
MLTSAPLWTVRSFVELSSTVRNKCAACYRQFNRMEHLVEHMRASHHSPHEPRCGVCGKHCRSLDALRDHLGFGASLPPKPACATA
FAAKGCPLCLAVFPSSSSLRAHGPTCHHSRAPVPSRGAMPRMPVGGVVALGCKMVGGGSDGTLDLCGRVCVIDENETIVFENFVRP
LLPVTHYRYETTGIRPEYLRDAPTVKMVQRQVEGILLNGEQPWKVRSSRGAARILVGHGLEHDLDALGMDYPAYLKRDTAEYPPLMK
TSARLMSNSLRFLTQSCLGYDIQTGHQHPYEDCVAAMRLYKRMRAMTHGPRKNGGEGDACAAVAFPARRQRELERMSPEELLSMS
KLDYHCWCLDD
SEQIDNO: 200
AGCCGGCTCAACTTATCCATTCATTAATCAAAGCGTTTGCTTCTGATCCCTTGATTTGGAAATCATAATTCCTTTGCATTTGGGGT
TTGAATTAGTCAACTGTTTCTATAATCCAATGCCTTTGCATTGTTTCTTCCTTTGGTTATGAACCGATGCTCACATCAGCTCCGTT
ATGGACCGTTAGATCCTTTGTTGAATTATCATCGACGGTCCGGAACAAGTGCGCGGCGTGCTACCGCCAGTTCAACAGGATGG
AGCACCTGGTGGAGCACATGCGCGCGTCGCACCACTCGCCGCACGAGCCGCGCTGCGGCGTCTGCGGGAAGCACTGCCGCT
CCCTCGACGCCCTCCGTGACCACCTCGGCTTCGGCGCCTCCCTGCCCCCGAAGCCCGCCTGCGCCACGGCCTTTGCTGCCAA
GGGCTGCCCGCTCTGCCTCGCCGTCTTCCCTAGCTCCAGCTCCCTCCGCGCCCACGGCCCAACCTGCCACCACTCCCGCGCT
CCGGTTCCCTCGAGGGGGGCTATGCCGAGAATGCCCGTCGGCGGCGTGGTGGCGCTGGGGTGCAAGATGGTGGGGGGCGG
GAGCGACGGGACGCTGGACCTGTGCGGGCGCGTCTGCGTCATCGACGAGAATGAGACCATCGTCTTCGAGAACTTTGTGAGG
CCGCTCCTCCCGGTGACGCACTACCGGTACGAGACCACGGGGATCCGCCCCGAGTACCTGCGGGACGCGCCGACGGTGAAG
ATGGTGCAGCGGCAGGTGGAGGGCATCCTCCTCAACGGCGAGCAGCCGTGGAAGGTCCGGTCCTCGCGCGGCGCGGCCAG
GATCCTCGTCGGCCACGGCCTGGAGCACGATCTCGACGCGCTGGGCATGGACTACCCGGCGTACCTGAAGCGGGACACGGC
GGAATATCCGCCGCTGATGAAGACGAGCGCCAGGCTGATGAGCAACTCGCTTCGGTTCCTCACACAAAGCTGCCTCGGCTAC
GACATCCAGACGGGCCACCAGCACCCCTACGAGGACTGCGTGGCGGCCATGCGGCTGTACAAGAGAATGCGCGCGATGACG
CACGGCCCGCGGAAGAACGGGGGCGAAGGCGATGCGTGCGCGGCGGTGGCATTCCCGGCGCGGAGGCAGCGGGAGCTGG
AGCGCATGTCGCCGGAGGAGCTCCTGAGCATGTCCAAGCTCGACTATCACTGCTGGTGCCTCGACGACTAGCCTCGACGACT
GCAGCTGCATCTGCATGTCCAACAGTCCAAGCATGAGCCATG
SEQIDNO: 201
ATGCTCACATCAGCTCCGTTATGGACCGTTAGATCCTTTGTTGAATTATCATCGACGGTGTCCTTCGTATACAAAAACCTCGAGA
AGTTCTTTCCCTAATACATAATGCCATTGGTAAATCATATTCTCTGATTTGGCCAACCATGCTCTGCTTTCGATAGTTTGGCCATG
TTTACAAAAAATATATCAACATCAACAACACCAAATTTTCATTAGACCCGCCGTAAAATATAAGAACAGAAGACACATTTTTTTTTC
AGTGAAGAAAACATATGATACTTTTAGCCTCATGACATGCCAAACTGAGCTTGAACAAGATCCGTCCAGTATGCCACGGTTGCG
CTCACCATGAACCTTCAGTTTCGTGAATCTATTCGACGTCGATTTGTAGTCACTGAAGATAGCAAACCAGATGCGCAGGGCCAG
GACCGTCCTTTGATCCCCGCCGAGAACGAGATGACAACGTCTCCAAAAGCAAAGCTAGCTCCGGAGGGTATGTTCCTAGGATA
TCCATGGTTGGATTCAGGGACCTGTGCAGCTCTCCGCCTCTCCTCCATCGCTAAAGCAAAGCCAATGATAGGATAATCTTTCCA
TGTGTTTGTTCTTGCCTTTTTGTTCAAACCGAATCTGTTGACCAGGGTATCTGATCTCTCCCCCCCTCTCTTTTCCTTTCTACATA
CAGCACCTGGTGTATCATGAGGGCGTTCTGGTGTCTATGTTCTTTTCTTTGCATGGGCTGGTGTCTATGTTCTTGATGCTCCAAG
AAAGATGAGGACATCTTGTGGCCTCTGTACTGTGCGCTGGTGAAGAAGGCAAGTCGCCATCAAGAATCGAATGCCATCGGTTTT
GCAGGCAGATAAATAGCCGTCCGTCCGTGTGCACAGATTCTTACAATCCATCCAACCAGTCTCAACCTCTGTTTGCTGCATACT
GCACAGCTTATTTGCTTAGGAAAGGTGACCGAGTGATCATGGATAGCTCTTCAGATGCCCACAGGTGCACTCACTGGCCACTAC
TCTGTTTTAGTTGTCTTGTTATCACACATTTTGCAGTCGAAGAACTCTGCTATACCTAAGCTGCAGTATGCTCAAAAGTTTTGTGA
GATCATGCCACCATAAATTCAGCACATTTTCTTTGTTTCTACATTACGTGACAAGTTGAGATTGATTCTTAAGAATTTTCTAATTAA
GCTGCTGCGTAGTGGACTGCATGATATAGAAAATGTAATAGATGATGTCGCTAAGATTGCACTGTGCGTTTTGTTGCCACAATG
CATGATCACTCAGGCAGAGACATACACCTATGAGTAGAGTAGTACCAGTGACGATGGATGATTCAATTCGTCGCGTGCATGCAG
CCGGAACAAGTGCGCGGCGTGCTACCGCCAGTTCAACAGGATGGAGCACCTGGTGGAGCACATGCGCGCGTCGCACCACTC
GCCGCACGAGCCGCGCTGCGGCGTCTGCGGGAAGCACTGCCGCTCCCTCGACGCCCTCCGTGACCACCTCGGCTTCGGCGC
CTCCCTGCCCCCGAAGCCCGCCTGCGCCACGGCCTTTGCTGCCAAGGGCTGCCCGCTCTGCCTCGCCGTCTTCCCTAGCTCC
AGCTCCCTCCGCGCCCACGGCCCAACCTGCCACCACTCCCGCGCTCCGGTTCCCTCGAGGGGGGCTATGCCGAGAATGCCC
GTCGGCGGCGTGGTGGCGCTGGGGTGCAAGATGGTGGGCGGCGGGAGCGACGGGACGCTGGACCTGTGCGGGCGCGTCTG
CGTCATCGACGAGAATGAGACCATCGTCTTCGAGAACTTTGTGAGGCCGCTCCTCCCGGTGACGCACTACCGGTACGAGACCA
CGGGGATCCGCCCCGAGTACCTGCGGGACGCGCCGACGGTGAAGATGGTGCAGCGGCAGGTGGAGGGCATCCTCCTCAACG
GCGAGCAGCCGTGGAAGGTCCGGTCCTCGCGCGGCGCGGCCAGGATCCTCGTCGGCCACGGCCTGGAGCACGATCTCGAC
GCGCTGGGCATGGACTACCCGGCGTACCTGAAGCGGGACACGGCGGAATATCCGCCGCTGATGAAGACGAGCGCCAGGCTG
ATGAGCAACTCGCTTCGGTTCCTCACACAAAGCTGCCTCGGCTACGACATCCAGACGGGCCACCAGCACCCCTACGAGGACTG
CGTGGCGGCCATGCGGCTGTACAAGAGAATGCGCGCGATGACGCACGGCCCGCGGAAGAACGGGGGCGAAGGCGATGCGT
GCGCGGCGGTGGCATTCCCGGCGCGGAGGCAGCGGGAGCTGGAGCGCATGTCGCCGGAGGAGCTCCTGAGCATGTCCAAG
CTCGACTATCACTGCTGGTGCCTCGACGACTAG
SEQIDNO: 202
MEHLVEHMRSSHHSHHEPRCGVCGKHCRSLDALRDHLGFGASLPSKPACAATFQAHGCPLCLAVFPTSAALRAHRPACKLSGAPH
PSSVQSLTRTMSRVGARGGRGAVALGCKMVGGGSDGTLDVCARVCVVDEHEAILYESFVKPLIPVTHYRYETTGIRPEHLRDAPTVK
QAMRRVQDILLNGEQSYYSSRGAARLLVGHGLEHDLDALGMDYPAHLRRDTAAYPPLMKTSARLMSNSLRYLTRSCLGYDIQTGGH
HHPYDDCVAAMRLYKRMRAMSHLHLHGRPKDDDDESAVKAFPAWRQRELERMSPEELLAMSKPDYRCWCLDDDRRC
SEQIDNO: 203
GGTAGCCGTCACAGTTGCGCGGCGTGCTACCGTCAGTTCAACCGGATGGAGCACCTGGTGGAGCACATGCGGTCGTCGCACC
ACTCCCACCACGAGCCCCGCTGCGGCGTCTGCGGCAAGCACTGCCGCTCCCTCGACGCCCTCCGCGACCACCTCGGCTTCG
GCGCCTCCCTGCCCTCCAAGCCCGCCTGCGCCGCCACCTTCCAAGCCCACGGCTGCCCGCTCTGCCTCGCCGTCTTCCCCAC
CTCCGCCGCCCTTCGCGCCCACCGCCCAGCATGCAAGCTCTCCGGCGCCCCCCATCCTTCCTCGGTGCAGAGCCTCACGAGG
ACTATGTCGAGGGTGGGCGCGCGAGGCGGCCGCGGCGCGGTGGCGCTGGGGTGCAAGATGGGGGGGGGGGGAGCGACG
GCACGCTGGACGTGTGCGCGCGCGTCTGCGTCGTCGACGAGCACGAGGCCATCCTCTACGAGAGCTTCGTGAAGCCCCTCAT
CCCGGTCACGCACTACCGGTACGAGACCACGGGCATCCGGCCCGAGCACCTGCGCGACGCGCCGACGGTGAAGCAGGCGAT
GAGGCGGGTCCAGGACATCCTCCTCAACGGCGAGCAATCTTATTACTCCTCCCGCGGCGCGGCCCGGCTCCTCGTCGGCCAC
GGGCTGGAGCACGACCTCGACGCGCTCGGCATGGACTACCCGGCGCACCTCAGGGGGGACACGGCCGCGTACCCGCCGCT
GATGAAGACGAGCGCCAGGCTCATGAGCAACTCGCTCCGGTATCTCACGCGGAGCTGCCTGGGCTATGACATACAGACCGGC
GGACACCATCACCCCTACGACGACTGCGTGGCCGCCATGCGCCTCTACAAGAGGATGCGCGCCATGAGTCACCTGCACCTGC
ACGGCCGGCCCAAGGACGACGATGATGAGTCCGCGGTCAAGGCCTTCCCGGCGTGGAGGCAGCGAGAGCTGGAGCGCATGT
CGCCGGAGGAGCTCCTGGCGATGTCCAAGCCCGACTACCGATGTTGGTGCCTCGACGACGACCGCCGATGCTGACGGCAGC
CTGCTCCCTACGGGATTCGAATCTTGGCTGGCGCTTTTACTTGGTGGAATGGTGGTGATGATGAAAAGATAAAGAGAAAATAAA
ATACCGCGTGCGTCGGATGGATAGTTGCCGTAGACGTGTGGTGGTCGAATAAATCACAGTTTTGCTTTGTGTGAGAAAAAAAAA
AAAAAACGA
SEQIDNO: 204
MDSRRESSETLRNKCAACYRQYNRMEHLVEHMKVSFHSAHEPRCGVCAKHCRSLESLREHLIGPLPKVECARVFAARGCSICLNLF
DSAAAVRYHRASTCQFTRAAPMPRGSYGGRAVAMACKMVGGGSDGSLDICARVCLIGEDENVIFQTYVKPTTTVTNYRYEMTGIRP
EYLRDAMPLKLVQRRIQDILCNGEPLWKIRPRSSGRARILVGHGLEHELERLGLEYPAFMIRDTAKYPPLMKTSKLSNSLKYLTQAYLG
YDIHTGIQDPYEDCVAAMRLYIRMRSQAHPRDYASGSGETQNNYPAWRQRELERMSPEELLALSGSDYYCWCLDF
SEQIDNO: 205
ATGGACAGCAGGAGGGAGTCCTCGGAGACCCTGAGGAACAAATGCGCGGCGTGCTACAGGCAGTACAACCGGATGGAGCAC
CTGGTGGAGCACATGAAGGTGTCGTTCCACTCCGCCCACGAGCCCCGCTGCGGCGTCTGCGCCAAGCACTGCCGCTCCCTCG
AGTCCCTCCGCGAGCACCTCATCGGGCCGTTGCCCAAGGTGGAGTGCGCGCGCGTGTTCGCGGCCCGCGGCTGCAGCATCT
GCCTCAACCTCTTCGACAGCGCCGCCGCCGTCAGATACCACCGCGCCTCCACCTGCCAGTTCACCCGCGCCGCCCCGATGCC
CAGGGGTAGCTACGGAGGCCGTGCGGTGGCCATGGCGTGTAAGATGGTCGGAGGAGGAAGCGATGGCTCGCTCGACATCTG
CGCGAGGGTGTGCCTGATCGGAGAGGACGAGAACGTCATCTTCCAGACCTATGTGAAACCCACCACGACCGTCACAAACTACA
GGTATGAAATGACTGGGATAAGGCCGGAGTACTTGAGGGACGCAATGCCACTGAAGCTTGTGCAGAGGAGGATCCAGGACAT
CCTGTGCAACGGTGAGCCACTATGGAAGATACGGCCAAGGAGCTCTGGGAGGGCAAGGATCCTCGTTGGCCATGGCCTGGAG
CATGAACTTGAGCGCCTGGGACTGGAGTACCCGGCATTCATGATCAGGGATACTGCAAAGTACCCACCACTGATGAAAACTAG
CAAGCTGAGCAACTCCCTAAAGTACCTTACACAGGCATATCTTGGGTATGATATCCACACTGGCATTCAGGATCCCTATGAAGA
TTGCGTGGCAGCGATGAGGCTGTACATTAGGATGAGATCGCAAGCCCACCCGAGAGACTACGCCTCCGGTTCAGGTGAGACG
CAGAACAACTACCCAGCCTGGAGGCAGAGGGAGCTAGAGAGGATGAGCCCAGAAGAACTCCTGGCACTCTCGGGTTCAGATT
ACTACTGTTGGTGCCTGGATTTCTGA
SEQIDNO: 206
ATTACGATAAACTGCAGGCTTATTCCTCAGAATGCATGCAGCAGTGACGACAAGACGGTGATTCCCAGGGGTCATTCCTTCTTC
CTCGTGAAGGTTCTTCCATCCTTTCAACGTTTGTTGCCTCTATAAATAGCCTCTCCATGTTGCCTACCGTCCTCACCAACCTAGC
TAACTAGCGCCCCTCGAGGTTTCAGGGAGACAACTGATCGATCAGAAACCTTGGTCGACCGATCAGTAAGAGCAACTAGAGAC
AGAAACCTTAATTCTCGATCAGCAGGAGTTGTAGTAGTGCTATATATGATGGACAGCAGGAGGGAGTCCTCGGAGACCCTGAG
GTAAGATGGTCACAAACTGAATGCCATTACTACAGTAGTATTGCAAGATGAGCTGAGCATGCAAGACTGGTGTGTGATGAACTA
GCTAATTAATCAAGCTTTAATCATGCATGGTAGTACGTTCTTCTCGTGCTCTAAACATCGAGCTAGATATATAGGACCCTTTTTTT
TCTTGTCCTAGCTAGTTCCATTCAATACCTTGAATATGCATGTATATATGATACTACATATTCTGCGCACGAATGAATGAGTACCT
TTTCAACTTTCTTATCCCCTATTGCCGGTGCAAAGAATCAGAATTTCATAAATGCGTGCATAGTGCCTCGCTCGCGATATTCTAAA
GACGCGATCGAACTCAATCGATGAATGAAAATGATGATCTCGCAGGAACAAATGCGCGGCGTGCTACAGGCAGTACAACCGGA
TGGAGCACCTGGTGGAGCACATGAAGGTGTCGTTCCACTCCGCCCACGAGCCCCGCTGCGGCGTCTGCGCCAAGCACTGCC
GCTCCCTCGAGTCCCTCCGCGAGCACCTCATCGGTGAGTACACAAGTAGCCGTTTTTAATTTGCTCGGTTGATGCAACGAGCTA
GTTTTTGTGGAGTTTTCGCGTGGTATCTGACGGTTTCTCGCGTACGCCGTGCGCATGCAGGGCCGTTGCCCAAGGTGGAGTGC
GCGCGCGTGTTCGCGGCCCGCGGCTGCAGCATCTGCCTCAACCTCTTCGACAGCGCCGCCGCCGTCAGATACCACCGCGCCT
CCACCTGCCAGTTCACCCGCGCCGCCCCGGTATGTAATCCCCTCTCACCTCAGTTACACTGCGCATGCTAGTTTGCAAGGCTT
GTTGCTGAATTTGCCCGTGGTTTTCTTGCGTTTGCATGCAGATGCCCAGGGGTAGCTACGGAGGCCGTGCGGTGGCCATGGC
GTGTAAGATGGTCGGAGGAGGAAGCGATGGCTCGCTCGACATCTGCGCGAGGGTGTGCCTGATCGGAGAGGACGAGAACGT
CATCTTCCAGACCTATGTGAAACCCACCACGACCGTCACAAACTACAGGTAAAATATATAGGAGATGATCCATTATTTTATTACT
GATGATGTTTCTGAGTGTTAGCTAGTAGTACAATTTTTAGCTGTCATTGTTCGCATGTGTAGATCTATCGGACCAGAAAAGCAGA
AAAGACATAATGGAAAACCGAGCAATAAACATACTGCTATAGAAACTAGCAGTAGAAATTAAAGGCATAGATCGATGGATAGTTT
TATGAGATGATGAATGAGGGGATGATTTATGATGTTTCTGTTCAGGTATGAAATGACTGGGATAAGGCCGGAGTACTTGAGGGA
CGCAATGCCACTGAAGCTTGTGCAGAGGAGGATCCAGGACATCCTGTGCAACGGTGAGCCACTATGGAAGATACGGCCAAGG
AGCTCTGGGAGGGCAAGGATCCTCGTTGGCCATGGCCTGGAGCATGAACTTGAGCGCCTGGGACTGGAGTACCCGGCATTCA
TGATCAGGTCAGCAGCACAAGGCAAATTAATTTGCTGCAAATTAGACCTTGCCAGTTTCTCCCTTGCTGACACAATTTATCTCTG
CATTTTTTTCAGGGATACTGCAAAGTACCCACCACTGATGAAAACTAGCAAGCTGAGCAACTCCCTAAAGTACCTTACACAGGCA
TATCTTGGGTATGCAAACTTGCTAAGTCAGCTGTACAATCTCTATAGACTCTTAGACTTTGCATTATTAATGTTGAAGATACTATC
CTTGAATCCCTATTGCAGGTATGATATCCACACTGGCATTCAGGATCCCTATGAAGATTGCGTGGCAGCGATGAGGCTGTACAT
TAGGATGAGATCGCAAGCCCACCCGAGAGACTACGCCTCCGGTTCAGGTGAGACGCAGAACAACTACCCAGCCTGGAGGCAG
AGGGAGCTAGAGAGGATGAGCCCAGAAGAACTCCTGGCACTCTCGGGTTCAGATTACTACTGTTGGTGCCTGGATTTCTGAAG
GCTGAACTGATCGGTTGAAGAGAATAAGGAAGAGCCAACATTGATGGTTTATTCATGTTTCGTCCAAATCTCTATATATTTATAAG
TATATCTTGGGACTTGATTATAGTGGAATTGACTATAATGTATAAAGTTATCCTATGTAATGCTATCATATAATATTGGTGAGTTAG
AGTTGTATGGAGTGATGTTAAACAG
SEQIDNO: 207
MDNSSDSQRRKRCAACYREFNKKEHLVEHMRTSLHSAHDPRCGVCGKHCRSLDALRDHLTGALPKPECAAAFASRGCPLCLHVVL
PPTAAAHSCPAAAPPLGGVLALGCKMVGAGSDGSLDVCARVCVVDEQERVVLDTFVKPHIPVTHYRYDTTGIRPEHLRDAMTPKQA
ARRVQELLLNGEPAWKARSSRGRARILVGHGLDHDLESLGMDYPEYLKRDTARYPALMKTSNSRLSNSLKYLTLAYLGYHIQIAGRH
HHPYDDCVAALRLYRRMRGARPHTCRDAGVGPHAPPPTPAEAFPAWRQRELERMSPEELLQLSTSDYYCWCLDATD
SEQIDNO: 208
ATGGATAATTCTTCAGATTCTCAGAGGAGGAAGAGGTGCGCGGCGTGCTATAGGGAGTTCAACAAGAAGGAGCACCTGGTGGA
GCACATGCGGACGTCGCTGCATTCGGCGCACGACCCTCGCTGCGGCGTCTGCGGCAAGCACTGCCGCTCCCTCGACGCCCT
CCGCGACCACCTCACCGGCGCCCTCCCCAAGCCGGAGTGCGCCGCCGCCTTCGCCTCCCGCGGCTGCCCCCTCTGCCTCCA
CGTAGTCCTCCCGCCCACCGCCGCCGCCCACTCCTGCCCCGCGGCCGCGCCACCGCTCGGCGGCGTCCTCGCCCTGGGGTG
CAAGATGGTGGGCGCCGGCAGCGACGGGTCCCTGGACGTGTGCGCCCGCGTGTGCGTGGTGGACGAGCAGGAGCGCGTGG
TGTTGGACACCTTCGTCAAGCCGCACATCCCCGTCACGCACTACCGCTACGACACCACCGGCATCCGCCCCGAGCACCTGCG
CGACGCCATGACGCCCAAGCAGGCGGCGCGCCGGGTGCAGGAGCTGCTGCTCAACGGCGAGCCGGCGTGGAAGGCGCGGA
GCAGCCGCGGGAGGGCCCGGATCCTGGTCGGCCACGGCCTGGACCACGACCTCGAGTCGCTGGGCATGGACTACCCGGAGT
ACCTGAAGCGGGACACGGCGAGGTACCCGGCGCTGATGAAGACGAGCAACAGCCGCCTCAGCAACTCGCTCAAGTACCTCAC
CCTCGCCTACCTCGGCTACCACATCCAGATCGCCGGCCGCCACCACCACCCCTACGACGACTGCGTCGCCGCGCTGCGCCTC
TACCGCCGGATGCGTGGCGCGCGGCCGCACACCTGCAGGGACGCCGGCGTGGGGCCGCACGCGCCGCCGCCAACGCCGG
CGGAGGCGTTCCCGGCGTGGAGGCAGCGGGAGCTGGAGCGCATGTCGCCGGAGGAGCTCCTCCAGCTGTCCACCTCGGACT
ACTACTGCTGGTGCCTCGACGCCACCGACTAA
SEQIDNO: 209
TTCTACTCCATCGGTTCGAAAATATAAACCATTTTGAAGAAATGTGACACATTATATACTATGAATCTGGATACGGAGCCTGTCCA
GATTCGTAGTAGAGGATATGTCAAAAACGGAGGGAGTATCTAGCAGGGACATCACATTCGATCGACAACACTGTCAAGAATCAA
ATTCTGTCCGTTGGATTTGCGTATAAATAGGCATCGGAAACAAGTGCAGATAGCAATCACCAATCAGCACAACAACTAACTACAC
CAGTGGCCACTCACCTGCACTCCTGCAGTAGTCTTCAGATCGAGAATGGATAATTCTTCAGATTCTCAGAGGTATTAGCCAATG
TTCTAACTACATTCTGATTATATATATATATATATATATGAAGAAAAGAAAGAAAGAAAGAAAGAAAGAAATTGATTAAGTTAAATA
AAAAATATGAATAGGAGGAAGAGGTGCGCGGCGTGCTATAGGGAGTTCAACAAGAAGGAGCACCTGGTGGAGCACATGCGGA
CGTCGCTGCATTCGGCGCACGACCCTCGCTGCGGCGTCTGCGGCAAGCACTGCCGCTCCCTCGACGCCCTCCGCGACCACCT
CACCGGCGCCCTCCCCAAGCCGGAGTGCGCCGCCGCCTTCGCCTCCCGCGGCTGCCCCCTCTGCCTCCACGTAGTCCTCCC
GCCCACCGCCGCCGCCCACTCCTGCCCCGCGGCCGCGCCACCGCTCGGCGGCGTCCTCGCCCTGGGGTGCAAGATGGTGG
GCGCCGGCAGCGACGGGTCCCTGGACGTGTGCGCCCGCGTGTGCGTGGTGGACGAGCAGGAGCGCGTGGTGTTGGACACC
TTCGTCAAGCCGCACATCCCCGTCACGCACTACCGCTACGACACCACCGGCATCCGCCCCGAGCACCTGCGCGACGCCATGA
CGCCCAAGCAGGCGGCGCGCCGGGTGCAGGAGCTGCTGCTCAACGGCGAGCCGGCGTGGAAGGCGCGGAGCAGCCGCGG
GAGGGCCCGGATCCTGGTCGGCCACGGCCTGGACCACGACCTCGAGTCGCTGGGCATGGACTACCCGGAGTACCTGAAGCG
GGACACGGCGAGGTACCCGGCGCTGATGAAGACGAGCAACAGCCGCCTCAGCAACTCGCTCAAGTACCTCACCCTCGCCTAC
CTCGGCTACCACATCCAGATCGCCGGCCGCCACCACCACCCCTACGACGACTGCGTCGCCGCGCTGCGCCTCTACCGCCGGA
TGCGTGGCGCGCGGCCGCACACCTGCAGGGACGCCGGCGTGGGGCCGCACGCGCCGCCGCCAACGCCGGCGGAGGCGTT
CCCGGCGTGGAGGCAGCGGGAGCTGGAGCGCATGTCGCCGGAGGAGCTCCTCCAGCTGTCCACCTCGGACTACTACTGCTG
GTGCCTCGACGCCACCGACTAATAACGAATTATTACTCCCACGTACTACCTAAACTACTTACTCAGTATAATTATGACTAATTATG
CTGCTGCGTGTCCGTCTTTATTAGTACTCCACTCCGTATGTACGAATCAGGATGTAAGACCATCGATCTGATAGAGACAGTACC
GTCACAGTACGTGTGACAACCACAGGTTGCAAAATTTTTTAAAAAAGGTTTT
SEQIDNO: 210
TGCATGTGACATTTAGACCTTATCGGAATTAATTTGTAGAATTATTAATTAAGATGTTGATTAGTTCAAACAAAAATTTTATATTAAA
AAATGTAAACGAATATTTTGTATGTTCAGTGAAAGTAAAACAAATTAAATTAACAAGAAACTTATAGAAGAAAATTTTTACTATTTA
AGAGAAAGAAAAAAATCTATCATTTAATCTGAGTCCTAAAAACTGTTATACTTAACAGTTAACGCATGATTTGATGGAGGAGCCAT
AGATGCAATTCAATCAAACTGAAATTTCTGCAAGAATCTCAAACACGGAGATCTCAAAGTTTGAAAGAAAATTTATTTCTTCGACT
CAAAACAAACTTACGAAATTTAGGTAGAACTTATATACATTATATTGTAATTTTTTGTAACAAAATGTTTTTATTATTATTATAGAAT
TTTACTGGTTAAATTAAAAATGAATAGAAAAGGTGAATTAAGAGGAGAGAGGAGGTAAACATTTTCTTCTATTTTTTCATATTTTCA
GGATAAATTATTGTAAAAGTTTACAAGATTTCCATTTGACTAGTGTAAATGAGGAATATTCTCTAGTAAGATCATTATTTCATCTAC
TTCTTTTATCTTCTACCAGTAGAGGAATAAACAATATTTAGCTCCTTTGTAAATACAAATTAATTTTCCTTCTTGACATCATTCAATT
TTAATTTTACGTATAAAATAAAAGATCATACCTATTAGAACGATTAAGGAGAAATACAATTCGAATGAGAAGGATGTGCCGTTTGT
TATAATAAACAGCCACACGACGTAAACGTAAAATGACCACATGATGGGCCAATAGACATGGACCGACTACTAATAATAGTAAGTT
ACATTTTAGGATGGAATAAATATCATACCGACATCAGTTTTGAAAGAAAAGGGAAAAAAAGAAAAAATAAATAAAAGATATACTAC
CGACATGAGTTCCAAAAAGCAAAAAAAAAGATCAAGCCGACACAGACACGCGTAGAGAGCAAAATGACTTTGACGTCACACCAC
GAAAACAGACGCTTCATACGTGTCCCTTTATCTCTCTCAGTCTCTCTATAAACTTAGTGAGACCCTCCTCTGTTTTACTCACAAAT
ATGCAAACTAGAAAACAATCATCAGGAATAAAGGGTTTGATTACTTCT
SEQIDNO: 211
MECADNPRNKCAACYRQFNRMEHLADHMRTSFHSVHEPTCGVCKKHCRSFESLREHLIGPLPKQECRNVFNIRGCKFCLAILDSPY
ALRVHQDRCQLSGVSHGISAYMANLGLRDSLTIDNGYSRGPQVVALACKTVGGGSDRSLDLCARVCIIDENENIIFHTYVKPSIPVTNY
RYETTGIRPEHLRDAMPLRQVQRKVQDFLCNGEPTWKIRSPKGGKARILVGHGLDHDLDKMQVEYPPIMIRDTAKYPPLMKTSKLSN
SLKYLTQAYLGYDIQNGIQDPYEDCVATMRLYVRMRRQVHRRQDYPLASDPQNRNNFASWRQNELERMSPEEMLAISRSDYYCWC
LDSA
SEQIDNO: 212
TAATAATATATACTTCACCTATATTTCATCACTAAAACCAGCTATGGAGTGTGCAGATAACCCAAGGAACAAATGTGCAGCTTGCT
ATAGACAGTTCAATAGGATGGAACATTTGGTTGATCATATGAGAACTTCGTTTCATTCAGTTCATGAACCTACTTGTGGTGTTTGT
AAAAAGCACTGCCGATCCTTTGAATCTCTAAGGGAACATCTAATAGGTCCATTGCCCAAACAAGAATGCAGGAACGTGTTCAAC
ATCCGAGGCTGCAAGTTTTGTTTAGCCATTCTCGATAGCCCTTATGCTCTTAGGGTTCATCAAGACAGATGCCAGCTCTCTGGA
GTGAGCCATGGGATATCAGCTTACATGGCTAACTTGGGTCTTAGAGATAGCTTAACAATCGACAATGGTTATTCAAGAGGCCCA
CAAGTTGTTGCACTTGCATGCAAAACTGTTGGTGGTGGAAGCGATAGGTCATTGGATCTTTGTGCAAGGGTTTGCATCATTGAT
GAAAATGAGAATATAATCTTCCATACTTATGTTAAACCTTCTATTCCAGTTACAAACTATAGGTACGAAACAACAGGCATTCGACC
AGAACATTTGAGGGATGCAATGCCATTGAGACAAGTTCAAAGAAAGGTTCAAGATTTCCTTTGCAATGGAGAACCAACGTGGAA
AATTCGATCACCTAAAGGTGGAAAAGCTAGGATTCTTGTAGGGCATGGTCTTGATCATGACCTAGATAAAATGCAAGTCGAATAT
CCACCAATTATGATAAGGGATACTGCAAAATATCCTCCCTTGATGAAAACAAGCAAACTTAGCAACTCACTCAAGTACTTAACTC
AAGCATATTTGGGGTATGACATTCAAAATGGCATTCAAGATCCTTATGAGGATTGTGTTGCAACAATGAGGCTTTACGTTAGGAT
GAGGAGACAAGTTCATAGGAGACAAGACTATCCGTTGGCTTCCGACCCTCAAAACCGGAACAACTTCGCATCGTGGAGGCAAA
ACGAGCTCGAGAGGATGTCCCCTGAAGAAATGTTGGCAATCTCAAGGTCTGATTACTACTGTTGGTGCTTGGATTCTGCGTAAA
AACACTGAGAAATGCCACTATTTCATGCT
SEQIDNO: 213
TAATAATATATACTTCACCTATATTTCATCACTAAAACCAGCTATGGAGTGTGCAGATAACCCAAGGTATGTCTCAATCTCAACCC
TAGACACATGCATATGAAACCCAACAATGTAGTAATTTTGTTGATGCAATGATCGAACAAGGCCGAGATTATGATGGTATTGGTC
GGTTCACCCCATTTGAGATGGGTCGGAGAGCCAGTTGTTCCGGTAATAGATAATGGAGGAAGTAAGCTCTCTCATGGAGAGCT
ATGAAGAAAGATGGAGAAGAGAAGAGAGGAAAAAGTCCCTTCTGCTGTTTCCTTGTCCCCTCTTTTTTCGTTTTCCGTAGGCTTT
GGTAATAATGGTCTAATACTTAGGTTGTCATAGTATTACGAGGATGTGACCGGAGATTTGATATGTATAATAATTTTATATATGTA
TAACAAGTTTCAATGATTAATGTTGTAATGCAGGAACAAATGTGCAGCTTGCTATAGACAGTTCAATAGGATGGAACATTTGGCT
GATCATATGAGAACTTCGTTTCATTCAGTTCATGAACCTACTTGTGGTGTTTGTAAAAAGCACTGCCGATCCTTTGAATCTCTAAG
GGAACATCTAATAGGTAACTAATTCAATTCATTTGTAATATATATATATATATATATGTCACAGATTCTTGAAAACTATATAAATTTG
TTAATAAATACAGGTCCATTGCCCAAACAAGAATGCAGGAACGTGTTCAACATCCGAGGCTGCAAGTTTTGTTTAGCCATTCTCG
ATAGCCCTTATGCTCTTAGGGTTCATCAAGACAGATGCCAGCTCTCTGGAGTGAGCCATGTATTAATTCAAGTCAAATTATCTTT
ATATACGAACATTTATACGTAGGTAAAAGTATCATAAAGGTCTCTGTACTAAAAGTTGGATTGCGTTTGTTCCCTCTACTCAAAAA
ATAAGCAAATTGGTACTTATACATAAGATCAAATAGTAAATTAGTCATTCTGTTGAAAATTTCATCTATTTCTACTGTTAAAAAATG
ATCTCTGTTGAAAATTTCATCTATTTCTACTGTTAAAAAATGATCTCTATACGTCAGAATGAAGTACATGTGGCACGCCATGTAGA
ATCGTCTAGTTATTCTATCAGTCTTGTCAATTTTTAACAGTAGAATTAGACAAAATTTTTAATAGAAAAAACTAGTTTGCTTTTTGAT
CTAATGTATAGGGATTAATTTACCCCCTTTTCTATAGTAAAGGAGGGCAAAATGCAATCTAACTCTTAATGCAGGAGACTCCATG
ATAATTTTATTATATGTATATTTATAATGGCACTTTTTTTCCCATATTGAAATTGATTTAAAATGTGGGTTTTACTTGTAATAGGGGA
TATCAGCTTACATGGCTAACTTGGGTCTTAGAGATAGCTTAACAATCGACAATGGTTATTCAAGAGGCCCACAAGTTGTTGCACT
TGCATGCAAAACTGTTGGTGGTGGAAGCGATAGGTCATTGGATCTTTGTGCAAGGGTTTGCATCATTGATGAAAATGAGAATAT
AATCTTCCATACTTATGTTAAACCTTCTATTCCAGTTACAAACTATAGGTATGCCCATAGGATAAATCCGAGTAATCCCTAATCTTT
TCATGTTCAACATATATTCAAACTTGGTCCGACACTTGCCTTAGTCTGAGTAACATAGGTTCCTTGTTTGAATTACTTTCAGGTAC
GAAACAACAGGCATTCGACCAGAACATTTGAGGGATGCAATGCCATTGAGACAAGTTCAAAGAAAGGTTCAAGATTTCCTTTGC
AATGGAGAACCAACGTGGAAAATTCGATCACCTAAAGGTGGAAAAGCTAGGATTCTTGTAGGGCATGGTCTTGATCATGACCTA
GATAAAATGCAAGTCGAATATCCACCAATTATGATAAGGTAAAAGATTAAGGATGCCATATATATATATATAACATACATTCAAAA
GGAATATATATGAGTCAAACTACAATCTTTTTTCAATACAGCTTGCAGGTTTTGTATTATTCGAACTCTTCAGTTTTCTTAAAGTAT
TCATGTTCGATATTTGTTTACAGGGATACTGCAAAATATCCTCCCTTGATGAAAACAAGCAAACTTAGCAACTCACTCAAGTACTT
AACTCAAGCATATTTGGGGTAAGTATCTATAGATCATCCATTTCTCCCCTATACGATCGGTATCCGATCGATGATCGTTAACGAG
AGAGTGATGCATCATGTTGGGTGTGAATTAGGTATGACATTCAAAATGGCATTCAAGATCCTTATGAGGATTGTGTTGCAACAAT
GAGGCTTTACGTTAGGATGAGGAGACAAGTTCATAGGAGACAAGACTATCCGTTGGCTTCCGACCCTCAAAACCGGAACAACTT
CGCATCGTGGAGGCAAAACGAGCTCGAGAGGATGTCCCCTGAAGAAATGTTGGCAATCTCAAGGTCTGATTACTACTGTTGGT
GCTTGGATTCTGCGTAAAAACACTGAGAAATGCCACTATTTCATTCTCCTCCCCCCCCCCAATTACTTG
SEQIDNO: 214
MECAGNPRNKCAACYRQFNRMEHLVEHMRTSFHSAHETTCGVCKKHSRSFESLRENLIGPLPKQECRNVFNIRGCKFCLAILDSPYA
LRVHQDRCQLSGVNHVLIQGISAYMANLGLRDSLTIDNGYSRGLQVVALACKTVGGGSDRSLDLCARVCIIDENENIIFHTYVKPPIPVT
NYRYETTGIRPEHLRDAMPLRQVQRKVQDFLCNGEPTWKIRSPKGGKARILVGHGLDHDLDKMQVEYPPIMIRDTAKYPPLMKTSKL
SNSLKYLTQAYLGYDIQNGIQDLYEDCVATMRLYVRMRRQIHRRQDYPLASDPQNRNNFASWRQNELERMSPVLFQ
SEQIDNO: 215
ATGGAGTGTGCAGGTAACCCAAGGAACAAATGTGCAGCTTGCTATAGACAGTTCAATAGGATGGAACATTTAGTTGAACATATG
AGAACTTCGTTTCATTCAGCTCATGAAACTACTTGTGGTGTTTGTAAAAAGCACAGCCGATCCTTTGAATCTCTAAGGGAAAATC
TAATAGGTCCATTGCCCAAACAAGAATGCAGGAACGTGTTCAACATCCGAGGCTGCAAGTTTTGTTTAGCCATTCTCGATAGCC
CTTATGCTCTTAGGGTTCATCAAGACAGATGCCAGCTCTCTGGAGTGAACCATGTATTAATTCAAGGGATATCAGCTTACATGGC
TAACTTGGGTCTTAGAGATAGCTTAACAATCGACAATGGTTATTCAAGAGGCCTACAAGTTGTTGCACTTGCATGCAAAACTGTT
GGTGGTGGAAGCGATAGGTCATTGGATCTTTGTGCAAGGGTTTGCATCATTGATGAAAATGAGAATATAATCTTCCATACTTATG
TTAAACCTCCTATTCCAGTCACAAACTATAGGTACGAAACAACAGGCATTCGACCAGAACATTTGAGGGACGCAATGCCATTGA
GACAAGTTCAAAGAAAGGTTCAAGATTTCCTTTGCAATGGAGAACCAACGTGGAAAATTCGATCACCTAAAGGTGGAAAAGCTA
GGATTCTTGTAGGGCATGGTCTTGATCATGACCTAGATAAAATGCAAGTCGAATATCCACCAATTATGATAAGGGATACTGCAAA
ATATCCTCCCTTGATGAAAACAAGCAAACTTAGCAACTCACTCAAATACTTAACTCAAGCATATTTGGGGTATGACATTCAAAATG
GCATTCAAGATCTTTATGAGGATTGTGTTGCAACAATGAGGCTTTACGTTAGGATGAGGAGACAAATTCATAGGAGACAAGACTA
TCCGTTGGCTTCCGACCCTCAAAACCGGAACAACTTCGCGTCGTGGAGGCAAAACGAGCTCGAGAGGATGTCCCCTGTGTTGT
TCCAATAG
SEQIDNO: 216
ATGGAGTGTGCAGGTAACCCAAGGTATGTATATGAAACCCAACAATTTTGTTGATGCAATGATCGAACAAGGCGGAGATTATGA
TGGTATTGGTCGGTTCACCCCATTTGAGATGGGTCGGAGAGCCAGTTGTTCTGGTAATAGATAATGGAGGAAGTAAGCTCTCTC
ATGGAGAGTTATGGAGAAAGATGGAGAAGAGAAGAGAGGAAAAAGTCCCTTCTGCTGTTTCCTTGTCCCCTCTTTTTTTTCCGTA
GGCTTTGGAAATAATGGTCTAATATACTTAGGTTGTCATAGAGTTACGAGGATGTGACCGGAGATTTGATATGTATAATAATTTTA
TATATGTATAACAAGTTTCAATAATTAATGTTGTAATGCAGGAACAAATGTGCAGCTTGCTATAGACAGTTCAATAGGATGGAACA
TTTAGTTGAACATATGAGAACTTCGTTTCATTCAGCTCATGAAACTACTTGTGGTGTTTGTAAAAAGCACAGCCGATCCTTTGAAT
CTCTAAGGGAAAATCTAATAGGTAACTAATTCAATTCATTTGTAATATATATATATATGTCACGGATTCTTGAAAACTATATAAATT
TGTTAATAAATACAGGTCCATTGCCCAAACAAGAATGCAGGAACGTGTTCAACATCCGAGGCTGCAAGTTTTGTTTAGCCATTCT
CGATAGCCCTTATGCTCTTAGGGTTCATCAAGACAGATGCCAGCTCTCTGGAGTGAACCATGTATTAATTCAAGTCAATTTGTCT
TTATAAACATACATGTATCCGTAGGTAAAAGTATCATAAATGTCTCTGTACTAAAAGTTGGATTGCATTTATTCCCTCTACTCAAAA
AACGAGCAAATTAGCACTTATACATAAGATCAAAGAGAAAACTAGTCCTTCTGTTAAAAATTTCATCCATTTCTACTGTTAAAAAAT
GATCTCTATACGTCAGAATGAAGTACACGTGGCACACCATGTAGAATTGTCTAGTTATTTTGTCAGTCACGTCAATTTTTGATAGT
AGAATTGGATAAAATTTTTAATAGAAAAGATTAGTTTGCTTTTTGATCTAATGTATATGGACCAATTTACCCCCTTTTCTTTAGTAA
AGAAGGACAAAATGTAATTTAACTCTTAGTGCAGGAGACTCCATGATACTTTTATTATATGTATATTTATAATGGCACATTTTTTCC
CATATTGAAATTGATTTAAAATGTGGGTTTTACTTGTAATAGGGGATATCAGCTTACATGGCTAACTTGGGTCTTAGAGATAGCTT
AACAATCGACAATGGTTATTCAAGAGGCCTACAAGTTGTTGCACTTGCATGCAAAACTGTTGGTGGTGGAAGCGATAGGTCATT
GGATCTTTGTGCAAGGGTTTGCATCATTGATGAAAATGAGAATATAATCTTCCATACTTATGTTAAACCTCCTATTCCAGTCACAA
ACTATAGGTATGCCCATAGGATAAATCCGAGTAATCCCTAATCTTTTCATGTTCAACATATATTCAAACTTGGTCCGACACTTGCC
TTAGTCTGAGTAACATGGGTTCCTTGTTTGAATTACTTTCAGGTACGAAACAACAGGCATTCGACCAGAACATTTGAGGGACGCA
ATGCCATTGAGACAAGTTCAAAGAAAGGTTCAAGATTTCCTTTGCAATGGAGAACCAACGTGGAAAATTCGATCACCTAAAGGT
GGAAAAGCTAGGATTCTTGTAGGGCATGGTCTTGATCATGACCTAGATAAAATGCAAGTCGAATATCCACCAATTATGATAAGGT
AAAAGTTTAAGGATGCTATATATATATATAACATACATTCAAAAGGAATATATATAAGTCAAACTACAATCTTTTTTCAATACAGCT
TGCAGGTTTTGTATTATTCGAACTCTTCAGTTTTCTTAAAGTATTCATGTTCGATATTTGTTTACAGGGATACTGCAAAATATCCTC
CCTTGATGAAAACAAGCAAACTTAGCAACTCACTCAAATACTTAACTCAAGCATATTTGGGGTAAGTATCTATAGATCAACCATTT
CTCCCCTATACGATCGGTATCCGATCGATGATCGTTAACGAGAGAGTGATGCATCATGTTGGGTGTGAATTAGGTATGACATTC
AAAATGGCATTCAAGATCTTTATGAGGATTGTGTTGCAACAATGAGGCTTTACGTTAGGATGAGGAGACAAATTCATAGGAGACA
AGACTATCCGTTGGCTTCCGACCCTCAAAACCGGAACAACTTCGCGTCGTGGAGGCAAAACGAGCTCGAGAGGATGTCCCCTG
TGTTGTTCCAATAG
SEQIDNO: 217
MECADNPRNKCAACYRQFNRMEHLVDHMRTLFHSVHEPTCGVCKKHCRSFESLREHLIGPLPKQECRNAFNIRGCKFCLAILDSPYA
LRGISAYMANLGLRDSLTIDNGYSRGPQVVALACKTVGGGSDRSLDLCARVCIIDEKENIIFHTYVKPPIPVTNYRYETTGIRPEHLRDA
MPLRQVQRKVQDFLCNGEPTWKIRSPKGGKARILVGHGLDHDLDKMQVEYPPIMIRDTAKYPPLMKTSKLSNSLKYLTQAYLGYDIQ
NGIQDPYEDCVATMRLYETRLPVGFRPSNRNNFASWRQNELERMSPEEMLAISSYLSNRHFNKCFGFNQVQFEMKTEFEYERKQN
EIEKICGSVEATCAPQYTVLPHMVSFSRCQIE
SEQIDNO: 218
ATGGAGTGTGCAGATAACCCAAGGAACAAATGTGCAGCTTGCTATAGACAGTTCAATAGGATGGAACATTTGGTTGATCATATG
AGAACTTTGTTTCATTCAGTTCATGAACCTACTTGTGGTGTTTGTAAAAAGCACTGCCGATCCTTTGAATCTCTAAGGGAACATCT
AATAGGTCCATTGCCCAAACAAGAATGCAGGAACGCGTTCAACATCCGAGGCTGCAAGTTTTGTTTAGCCATTCTCGATAGCCC
TTATGCTCTTAGGGGGATATCAGCTTACATGGCTAACTTGGGGCTTAGAGATAGCTTAACAATCGACAATGGTTATTCAAGAGG
CCCACAAGTTGTTGCACTTGCATGCAAAACTGTTGGTGGTGGAAGCGATAGGTCATTGGATCTTTGTGCAAGGGTTTGCATCAT
TGATGAAAAGGAGAATATAATCTTCCATACTTATGTTAAACCTCCTATTCCAGTCACAAACTATAGGTACGAAACAACAGGCATTC
GACCAGAACATTTAAGGGATGCAATGCCATTGAGACAAGTTCAAAGAAAGGTTCAAGATTTCCTTTGCAATGGAGAACCAACGT
GGAAAATTCGATCACCTAAAGGTGGAAAAGCTAGGATTCTTGTAGGGCATGGTCTTGATCATGACCTAGATAAAATGCAAGTCG
AATATCCACCAATAATGATAAGGGATACTGCAAAATATCCTCCCTTGATGAAAACAAGCAAACTTAGCAACTCACTCAAGTACTTA
ACTCAAGCATATTTGGGGTATGACATTCAAAATGGCATTCAAGATCCTTATGAGGATTGTGTTGCAACAATGAGGCTTTACGAGA
CAAGACTACCCGTTGGCTTCCGACCCTCAAACCGGAACAACTTCGCGTCGTGGAGGCAAAACGAGCTCGAGAGGATGTCCCCT
GAAGAAATGTTGGCAATCTCAAGTTATTTAAGTAATAGGCATTTTAATAAGTGCTTTGGCTTTAATCAGGTCCAATTTGAAATGAA
GACCGAGTTTGAATACGAGCGAAAACAAAATGAGATAGAAAAAATATGCGGGAGTGTCGAAGCAACATGTGCGCCACAGTACA
CTGTTCTGCCGCATATGGTTTCGTTTAGTCGCTGCCAAATTGAATGA
SEQIDNO: 219
ATGGAGTGTGCAGATAACCCAAGGTATGTCTCAATCTCAACCCTAGACATATGTATATGAAACCCAACAATGTAGTAATTTTGTT
GATGCAATGATCGAACAAGGCGGAGATTATGATGGTATTGGTCGGTTCACCCCATTTGAGATGGGTCGGAGAGCCGGTTGTTC
TGGTAATAGATAATGGAGGAAGTAAGCTCTCTCATGGAGAGTTATGGAGAAAGATGGAGAAGAGAAGAGAGGAAAAAGTCCCTT
CTGCTGTTTCCTTGTCCCCTCTTTTTTTTCCCGTAGGCTTTGGTAATAATGGTCTAATATACTTAGGTTGTCATAGAGTTACGAGG
ATGTGACCGGAGATTTGATATGTATAATAATTTTATATATGTATGACAAGTTTCAATGATTAATGTTGTAATGCAGGAACAAATGT
GCAGCTTGCTATAGACAGTTCAATAGGATGGAACATTTGGTTGATCATATGAGAACTTTGTTTCATTCAGTTCATGAACCTACTTG
TGGTGTTTGTAAAAAGCACTGCCGATCCTTTGAATCTCTAAGGGAACATCTAATAGGTAACTAATTCAATTCATTTGTAATATATA
TATGTCACGGATTCTTGAAAACTATATAAATTTGTTAATAAATACAGGTCCATTGCCCAAACAAGAATGCAGGAACGCGTTCAAC
ATCCGAGGCTGCAAGTTTTGTTTAGCCATTCTCGATAGCCCTTATGCTCTTAGGGTTCATCAAGACAGATGCCAGCTCTCTGGA
GTGAACCATGTATTAATTCAAGTCAAATTGTCTTTATATACATACATATATACGTAGATAAAAGTATCATAAAGGTCCTATACTAAA
AGTTGGATTGTGTTTATTCTCTCTACTCAAAAATGAGTAAATTAGTACTTATATATAAGATCAAAGAGTAAACTAGTCCTTCTGTTA
AAAATTTCATACATTTCTACTGTTAAAAAATGATCTCTATACGTCAGAATAAAGTACATGTGGCACGTCATGTAGAATTGTCTAGT
TATTCTGTCAGCCACGTCAATTTTTAACAGTAAAATTGGATAAATTTTTTAATAGAAAAGACTAGTTTTCTTTTTGATCTAATGTATA
GGAACCAATTTACCCCTTTTTCTTTAGTAAAGGAGGGCAAAATGTAATCTAACTTTTAGTGCAGGAGACTCCATGATACTTCTATT
GTATGTATATTTATAATGGCACTTTTTTTCCCATATTGAAATTGATTTAAAATGTGGGTTTTACTTGTAATAGGGGATATCAGCTTA
CATGGCTAACTTGGGGCTTAGAGATAGCTTAACAATCGACAATGGTTATTCAAGAGGCCCACAAGTTGTTGCACTTGCATGCAA
AACTGTTGGTGGTGGAAGCGATAGGTCATTGGATCTTTGTGCAAGGGTTTGCATCATTGATGAAAAGGAGAATATAATCTTCCAT
ACTTATGTTAAACCTCCTATTCCAGTCACAAACTATAGGTATGCCCATAGGATAAATCCGAGTAATCCCTAATCTTTTCATGTTCA
ACATATATTCAAACTTGGTCCGATACTTGCCTTAGTCTGAGTAACATAGGTTCCTTGTTTGAATTACTTTCAGGTACGAAACAACA
GGCATTCGACCAGAACATTTAAGGGATGCAATGCCATTGAGACAAGTTCAAAGAAAGGTTCAAGATTTCCTTTGCAATGGAGAA
CCAACGTGGAAAATTCGATCACCTAAAGGTGGAAAAGCTAGGATTCTTGTAGGGCATGGTCTTGATCATGACCTAGATAAAATG
CAAGTCGAATATCCACCAATAATGATAAGGTAAAAGTTTAAGGATGCCATATATATATATAACATACATTCAAAAGGAATATATAT
AAGTCAAACTACAATCTTTTTTCAATACAGCTTGCAGGTTTTGTATTATTCGAACTCTTCGGTTTTCTTAAAGTATTCATGTTCGAT
ATTTGTTTACAGGGATACTGCAAAATATCCTCCCTTGATGAAAACAAGCAAACTTAGCAACTCACTCAAGTACTTAACTCAAGCAT
ATTTGGGGTAAGTGTCTATAGATCATCCATTTCTCCCCTATACGATCGGTATCCGATCGACGATCGTTAACGAGAGAGTGATGC
ATCATGTTGGGTGTGAATTAGGTATGACATTCAAAATGGCATTCAAGATCCTTATGAGGATTGTGTTGCAACAATGAGGCTTTAC
GTTAGGATGAGGAGACAAGTTCATAGGAGACAAGACTACCCGTTGGCTTCCGACCCTCAAACCGGAACAACTTCGCGTCGTGG
AGGCAAAACGAGCTCGAGAGGATGTCCCCTGAAGAAATGTTGGCAATCTCAAGGTCTGATTACTACTGTTGGTGCTTGGATTCT
GCGTAAAGACACTGAGAAATGCCACTATTTCATTCTCCTCCCCCCCCCCAATTACTTGTTATAGCAATAAGAAGGAAGTAGTTCA
TTGTTATTTCCCTAGTGTTTATTTGTACTAGTAAACAATTTATTGGATTATATATACATGTATGTATGCTAGTGAAATCATCTAAATA
AATGGAATTATAAGAGTATTCATAGAGTAAGTTTAGCTCGGTTGGTTTATAGACTACAGTCCTCTTTCAAAAAAAAAAAAACTGAA
ATTCCAAAATAAACATGATAAATACATTAATAGACACATTAAAAGTTTAAAATAACTAATAAGAAAGCATACAAACTTAACAAAATT
AAACTGAATGAATAATCAAAATATTATGAATTTAACAATTGAAATAACATTCAAATATTAAATTTCGGTTAATTTGAGTGAACTGATT
ATTTTTTTATAATTATCTGAACTGAATGAAGTTTATTCTGTTAAATAATCAACCTATAGATACCGTAAAAAATTCCAACAAGAACAC
AAATTCAACATATATAAAGAGATTATGAAATATGAATCGTCATAACAATATTATTTCAATTCTCTAATAAATTTGCAATTTTCAGCTG
CCTGTTTATCTTGGGTTTTTCTCCCAGGATGATGATCAATGTTGGGAAATGTGGACAACTTTTTCATATGTTTTAGTTGAGAGTTT
GATATTTACCATGCATTTGGCACCATCTTCACCTCATGGTTAATATTGATTCCCTTTTTGTGAGGACCACTCGACACCTACAACTT
CATCTTTGAATCTCATTGGTTTCAAATGGAATCAGATTTCCATAAAAGTTCATCCTCTGATTTTTCTTGGCTTTCTTGATCAAACAC
CACCATCAAAGTCTTCATCATATAAATATATTTGTACTTTTTTCACTTATGAGTTTTTAAGTAAATATTATATTTAATTGCATAATTAG
GATAAATTTCATAGGTTGCATCATATTTATATTTTATGTTGTTTTACTTATTTTTAGTTATTTAAGTAATAGGCATTTTAATAAGTGCT
TTGGCTTTAATCAGGTCCAATTTGAAATGAAGACCGAGTTTGAATACGAGCGAAAACAAAATGAGATAGAAAAAATATGCGGGA
GTGTCGAAGCAACATGTGCGCCACAGTACACTGTTCTGCCGCATATGGTTTCGTTTAGTCGCTGCCAAATTGAATGA
SEQIDNO: 220
MECADNPRNKCAACYRQFNRMEHLVDHMRTSFHSVHEPTCGVCKKHCRSFESLREHLIGPLPKQECRNAFNIRGCKFCLAILDSPY
ALRVHQDRCQLSGVNHGISAYMANLGLRDSLTIDNGYSRGPEVVALACKTVGGGSDRSLDLCARVCIIDEKENIIFHTYVKPPIPVTNY
RYETTGIRPEHLRDAMPLRQVQRKVQDFLCNGEPTWKIRSPKGGKARILVGHGLDHDLDKMQVEYPPKMIRDTAKYPPLMKTSKLSN
SLKYLTQAYLGYDIQNGIQDPYEDCVATMRLYVRMRRQVHRRQDYPLASDPQNRNNFASWRQNELERMSPEEMLAISRSDYYCWC
LDSA
SEQIDNO: 221
ATGGAGTGTGCAGATAACCCAAGGAACAAATGTGCAGCTTGCTATAGACAGTTCAATAGGATGGAACATTTGGTTGATCATATG
AGAACTTCGTTTCATTCAGTTCATGAACCTACTTGTGGTGTTTGTAAAAAGCACTGCCGATCCTTTGAATCTCTAAGGGAACATCT
AATAGGTCCATTGCCCAAACAAGAATGCAGGAACGCGTTCAACATCCGAGGCTGCAAGTTTTGTTTAGCCATTCTCGATAGCCC
TTATGCTCTTAGGGTTCATCAAGACAGATGCCAGCTCTCTGGAGTGAACCATGGGATATCAGCTTACATGGCTAACTTGGGGCT
TAGAGATAGCTTAACAATCGACAATGGTTATTCAAGAGGCCCAGAAGTTGTTGCACTTGCATGCAAAACTGTTGGTGGTGGAAG
CGATAGGTCATTGGATCTTTGTGCAAGGGTTTGCATCATTGATGAAAAGGAGAATATAATCTTCCATACTTATGTTAAACCTCCTA
TTCCAGTCACAAACTATAGGTACGAAACAACAGGCATTCGACCAGAACATTTAAGGGATGCAATGCCATTGAGACAAGTTCAAA
GAAAGGTTCAAGATTTCCTTTGCAATGGAGAACCAACGTGGAAAATTCGATCACCTAAAGGTGGAAAAGCTAGGATTCTTGTAG
GGCATGGTCTTGATCATGACCTAGATAAAATGCAAGTCGAATATCCACCAAAAATGATAAGGGATACTGCAAAATATCCTCCCTT
GATGAAAACAAGCAAACTTAGCAACTCACTCAAGTACTTAACTCAAGCATATTTGGGGTATGACATTCAAAATGGCATTCAAGAT
CCTTATGAGGATTGTGTTGCAACAATGAGGCTTTACGTTAGGATGAGGAGACAAGTTCATAGGAGACAAGACTACCCGTTGGCT
TCCGACCCTCAAAACCGGAACAACTTCGCGTCGTGGAGGCAAAACGAGCTCGAGAGGATGTCCCCTGAAGAAATGTTGGCAAT
CTCAAGGTCTGATTACTACTGTTGGTGCTTGGATTCTGCATAA
SEQIDNO: 222
ATGGAGTGTGCAGATAACCCAAGGTATGTCTCAATCTCAACCCTAGACATATGTATATGAAACCCAACAATGTAGTAATTTTGTT
GATGCAATGATCGAACAAGGCGGAGATTATGATGGTATTGGTCGGTTCACCCCATTTGAGATGGGTCGGAGAGCCGGTTGTTC
TGGTAATAGATAATGGAGAAAGTAAGCTCTCTCATGGAGAGTTATGGAGAAAGATGGAGAAGAGAAGAGAGGAAAAAGTCCCTT
CTGCTGTTTCCTTGTCCCCTCTTTTTTTTCCCGTAGGCTTTGGTAATAATGGTCTAATATACTTAGGTTGTCATAGAGTTACGAGG
ATGTGACCGGAGATTTGATACGTATAATAATTTTATATATGTATGACAAGTTTCAATGATTAATGTTGTAATGCAGGAACAAATGT
GCAGCTTGCTATAGACAGTTCAATAGGATGGAACATTTGGTTGATCATATGAGAACTTCGTTTCATTCAGTTCATGAACCTACTT
GTGGTGTTTGTAAAAAGCACTGCCGATCCTTTGAATCTCTAAGGGAACATCTAATAGGTAACTAATTCAATTCATTTGTAATATAT
ATACATATATATATATATATGTCACGGATTCTTGAAAACTATATAAATTTGTTAATAAATACAGGTCCATTGCCCAAACAAGAATGC
AGGAACGCGTTCAACATCCGAGGCTGCAAGTTTTGTTTAGCCATTCTCGATAGCCCTTATGCTCTTAGGGTTCATCAAGACAGAT
GCCAGCTCTCTGGAGTGAACCATGTATTAATTCAAGTCAAATTGTCTTTATATACATACATATATACGTAGGTAAAAGTATCATAA
AGGTCCTGTACTAAAAGTTGGATTGTGTTTATTCCCTCTACTCAAAAAATGAGCAAATTAGTACTTATACATAAGACCAAAGAGTA
AACTAGTCCTTCTGTTGAAAATTTCATACATTTCTACTGTTAAAAAATGATCTCTATACGTCAGAATGAAGTACATGTGGCACGTC
ATGTAGAATTGTCTAGTTATTCTGTCAGCCACGTCAATTTTTAACAGTAGAATTGGATAAATTTTTAATAGAAAAGACTAGTTTTCT
TTTTGATCTAATGCATAGGGACCAATTTACCCCCTTTTCTTTAGTAAAGGAGGGCAAAATGTAATCTAACTTTTAGTGCAGGGGA
CTCCATGATACTTCTATGTATATTTATAATGGAACTTTTTTTCCCATATTGAAATTGATTTAAAATGTGGGTTTTACTTGTAATAGG
GGATATCAGCTTACATGGCTAACTTGGGGCTTAGAGATAGCTTAACAATCGACAATGGTTATTCAAGAGGCCCAGAAGTTGTTG
CACTTGCATGCAAAACTGTTGGTGGTGGAAGCGATAGGTCATTGGATCTTTGTGCAAGGGTTTGCATCATTGATGAAAAGGAGA
ATATAATCTTCCATACTTATGTTAAACCTCCTATTCCAGTCACAAACTATAGGTATGCCCATAGGATAAATCCGAGTAATCCCTAA
TCTTTTCATGTTCAACATATATTCAAACTTGGTCCGATACTTGCCTTAGTCTGAGTAACATAGGTTCCTTGTTTGAATTACTTTCAG
GTACGAAACAACAGGCATTCGACCAGAACATTTAAGGGATGCAATGCCATTGAGACAAGTTCAAAGAAAGGTTCAAGATTTCCT
TTGCAATGGAGAACCAACGTGGAAAATTCGATCACCTAAAGGTGGAAAAGCTAGGATTCTTGTAGGGCATGGTCTTGATCATGA
CCTAGATAAAATGCAAGTCGAATATCCACCAAAAATGATAAGGTAAAAGTTTAAGGATGCCATATATATATATATATAACATACAT
TCAAAAGGAATATATATAAGTCAAACTACAATCTTTTTTCAATACAGCTTGCAGGTTTTGTATTATTCGAACTCTTCGGTTTTCTTA
AAGTATTCATGTTCGATATTTGTTTACAGGGATACTGCAAAATATCCTCCCTTGATGAAAACAAGCAAACTTAGCAACTCACTCAA
GTACTTAACTCAAGCATATTTGGGGTAAGTATCTATAGATCATCCATTTCTCCCCTATACGATCGGTATCCGATCGATGATCGTTA
ACGAGAGAGTGATGCATCATGTTGGGTGTGAATTAGGTATGACATTCAAAATGGCATTCAAGATCCTTATGAGGATTGTGTTGCA
ACAATGAGGCTTTACGTTAGGATGAGGAGACAAGTTCATAGGAGACAAGACTACCCGTTGGCTTCCGACCCTCAAAACCGGAA
CAACTTCGCGTCGTGGAGGCAAAACGAGCTCGAGAGGATGTCCCCTGAAGAAATGTTGGCAATCTCAAGGTCTGATTACTACT
GTTGGTGCTTGGATTCTGCATAA
SEQIDNO: 223
MECAGNPRNKCAACYRQFNRMEHLVEHMRTSFHSVHEPTCGVCKKHCRSFESLREHLIGPLPKQECRNVFNIRGCKFCLAILDSPY
ALRVHQDRCQLSGVNHGISAYMANLGLRDSLTIDNGYSRGPQVVALACKTVGGGSDRSLDLCARVCIIDENENIIFHTFVKPPIPVTNY
RYETTGIRPEHLRDAMPLRQVQRKVQDFLCNGEPTWKIRSPKGGKARILVGHGLDHDLDKMQVEYPPIMIRDTAKYPPLMKTSKLSN
SLKYLTQAYLGYDIQNGIQDPYEDCVATMRLYVRMRRQVHRRQDYPLASDPQNRNNFASWRQNELERMSPEEMLAISRSDYYCWC
LDSA
SEQIDNO: 224
ATGGAGTGTGCAGGTAACCCAAGGAACAAATGTGCAGCTTGCTATAGACAGTTCAATAGGATGGAACATTTAGTTGAACATATG
AGAACTTCGTTTCATTCAGTTCATGAACCTACTTGTGGTGTTTGTAAAAAGCACTGCCGATCCTTTGAATCTCTAAGGGAACATCT
AATAGGTCCATTGCCCAAACAAGAATGCAGGAACGTGTTCAACATCCGAGGCTGCAAGTTTTGTTTAGCCATTCTCGATAGCCC
TTATGCTCTTAGGGTTCATCAAGACAGATGCCAGCTCTCTGGAGTGAACCATGGGATATCAGCTTACATGGCTAACTTGGGTCT
TAGAGATAGCTTAACAATCGACAATGGTTATTCAAGAGGCCCACAAGTTGTTGCACTTGCATGCAAAACTGTTGGTGGTGGAAG
CGATAGGTCATTGGATCTTTGTGCAAGGGTTTGCATCATTGATGAAAATGAGAATATAATCTTCCATACTTTTGTTAAACCTCCTA
TTCCAGTCACAAACTATAGGTACGAAACAACAGGCATTCGACCAGAACATTTGAGGGACGCAATGCCATTGAGACAAGTTCAAA
GAAAGGTTCAAGATTTCCTTTGCAATGGAGAACCAACGTGGAAAATTCGATCACCTAAAGGTGGAAAAGCTAGGATTCTTGTAG
GGCATGGTCTTGATCATGACCTAGATAAAATGCAAGTCGAATATCCACCAATTATGATAAGGGATACTGCAAAATATCCTCCCTT
GATGAAAACAAGCAAACTTAGCAACTCACTCAAATACTTAACTCAAGCATATTTGGGGTATGACATTCAAAATGGCATTCAAGAT
CCTTATGAGGATTGTGTTGCAACAATGAGGCTTTACGTTAGGATGAGGAGACAAGTTCATAGGAGACAAGACTATCCGTTGGCT
TCCGACCCTCAAAACCGGAACAACTTCGCGTCGTGGAGGCAAAACGAGCTCGAGAGGATGTCCCCTGAAGAAATGTTGGCAAT
CTCAAGGTCTGATTACTACTGTTGGTGCTTGGATTCTGCGTAA
SEQIDNO: 225
ATGGAGTGTGCAGGTAACCCAAGGTATGTATATGAAACCCAACAATGTAGTAATTTTGTTGATGCAATGATCGAACAAGGCGGA
GATTATGATGGTATTGGTCGGTTCACCCCATTTGAGATGGGTCGGAGAGCCAGTTGTTCTGGTAATAGATAATGGAGGAAGTAA
GCTCTCTCATGGAGAGTTATGGAGAAAGATGGAGAAGAGAAGAGAGGAAAAAGTCCCTTCTGCTGTTTCCTTGTCCCCTCTTTT
TTTTTCCGTAGGCTTTGGAAATAATGGTCTAATATACTTAGGTTGTCATAGAGTTACGAGGATGTGACCGGAGATTTGATATGTAT
AATAATTTTATATATGTATAACAAGTTTCAATGATTAATGTTGTAATGCAGGAACAAATGTGCAGCTTGCTATAGACAGTTCAATA
GGATGGAACATTTAGTTGAACATATGAGAACTTCGTTTCATTCAGTTCATGAACCTACTTGTGGTGTTTGTAAAAAGCACTGCCG
ATCCTTTGAATCTCTAAGGGAACATCTAATAGGTAACTAATTCAATTCATTTGTAATATATATATATATATATATGTCACGGATTCT
TGAAAACTATATAAATTTGTTAATAAATACAGGTCCATTGCCCAAACAAGAATGCAGGAACGTGTTCAACATCCGAGGCTGCAAG
TTTTGTTTAGCCATTCTCGATAGCCCTTATGCTCTTAGGGTTCATCAAGACAGATGCCAGCTCTCTGGAGTGAACCATGTATTAA
TTCAAGTCAATTTGTCTTTATAAACATACATGTATCCGTAGGTAAAAGTATCATAAATGTCTCTATACTAAAAGTTGGATTGCATTT
ATTCCCTCTACTCAAAAAACGAGCAAATTAGCACTTATACATAAGATCAAAGAGAAAACTAATCCTTCTGTTAAAAATTTCATCCA
TTTCTACGGTTAAAAAATGATCTCTTTACGTCAGAATGAAGTACACGTGGCACACCATGTAGAATTGTCTAGTTATTTTGTCAGTC
ACGTCAATTTTTGATAGTAGAGTTGGATAAAATTTTTAATAGAAAAGACTAGTTTGCTTTTTGATCTAATGTATATGGACCAATTTA
CCCCCTTTTCTTTAGTAAAGAAGGACAAAATGTAATTTAACTCTTAGTGCAGGAGACTCCATGATACTTTTATTATATGTATATTTA
TAATGGCACATTTTTTCCCATGTTGAAATTGATTTAAAATGTGGGTTTTACTTGTAATAGGGGATATCAGCTTACATGGCTAACTT
GGGTCTTAGAGATAGCTTAACAATCGACAATGGTTATTCAAGAGGCCCACAAGTTGTTGCACTTGCATGCAAAACTGTTGGTGG
TGGAAGCGATAGGTCATTGGATCTTTGTGCAAGGGTTTGCATCATTGATGAAAATGAGAATATAATCTTCCATACTTTTGTTAAAC
CTCCTATTCCAGTCACAAACTATAGGTATGCCCATAGGATAAATCCGAGTAATCCCTAATCTTTTCATGTTCAACATATATTCAAA
CTTGGTCCGACACTTGCCTTAGTCTGAGTAACATGGGTTCCTTGTTTGAATTACTTTCAGGTACGAAACAACAGGCATTCGACCA
GAACATTTGAGGGACGCAATGCCATTGAGACAAGTTCAAAGAAAGGTTCAAGATTTCCTTTGCAATGGAGAACCAACGTGGAAA
ATTCGATCACCTAAAGGTGGAAAAGCTAGGATTCTTGTAGGGCATGGTCTTGATCATGACCTAGATAAAATGCAAGTCGAATATC
CACCAATTATGATAAGGTAAAAGTTTAAGGATGCTATATATATATATAACATACATTCAAAAGGAATATATATAAGTCAAACTACAA
TCTTTTTTCAATACAGCTTGCAGGTTTTGTATTATTCGAACTCTTCAGTTTTCTTAAAGTATTCATGTTCGATATTTGTTTACAGGG
ATACTGCAAAATATCCTCCCTTGATGAAAACAAGCAAACTTAGCAACTCACTCAAATACTTAACTCAAGCATATTTGGGGTAAGTA
TCTATAGATCAACCATTTCTCCCCTATACGATCGGTATCCGATCGATGATCGTTAACGAGAGAGTGATGCATCATGTTGGGTGTG
AATTAGGTATGACATTCAAAATGGCATTCAAGATCCTTATGAGGATTGTGTTGCAACAATGAGGCTTTACGTTAGGATGAGGAGA
CAAGTTCATAGGAGACAAGACTATCCGTTGGCTTCCGACCCTCAAAACCGGAACAACTTCGCGTCGTGGAGGCAAAACGAGCT
CGAGAGGATGTCCCCTGAAGAAATGTTGGCAATCTCAAGGTCTGATTACTACTGTTGGTGCTTGGATTCTGCGTAA
SEQIDNO: 226
KKCAACFREFNKMEHLVDHMRTSYHSLHEPTCVLCNKHCRSFDSLREHLIGPLPKQECNKLFKILGCKFCLSILESPHALKLHQHRCR
FSGVNYGTMSRPANKSTTVVDNGFSSHVVALACQMVDGGGNNESMDGCARVCMVDEYENIIFHVYVKPPISVPNYRYENSGIGGEH
LRDGMPLKQVQRRIEEFLCNGEAMWKIRSPKAGKARILVGHHLHPLLQSLHLQYPSFMIRDTAAYPPLMKTNKLSNSLKYLTQTYLGY
DIQAGVQDPYEDCVATMRLYLRMRNQVHQREDYPQASDPRNRNNFAPSRQSELERMSPEAMLAISRSDYYCWCLDSM
SEQIDNO: 227
GCATTCGGTATTCTTTCCATCTTACTCCTTCACACACACACCATCCTATGCACATTTATGTCACTTCGACTCTTTATTTTTCTAATG
GGATAAACTAGCATTTAGTTCTTGAATTTGATAAATTTTACCAATTTGATCCTTGAATGTTTTTTGTCCACATTAGTCTTTGAATTTG
ACAACTTTTCTTAATTTTAGTCTATGCGATTACGCGGCACTCTGAGAGCGTTGCATTATCACATGGAGCAAAATTGAGAAAATTT
GTCAAGTTCTAGAACTAATGTGACCACAACAAAATTCAAGGACTTTTTTTTTTTTTAAATGTCAACTTTAGAGACTAAATGTTGTTT
TGTCCTATATTAAATACATTCATGTTTAACAATTAGATTCGATACATATTGTTTAATTTTCTTTTAAAAAAAAGAAGAAAGACAACAA
ATTAGGTTAAAACTATTGTAAATTAAAGCCCTCGAGGTAGTTTACTTTTGTTAAACAAACCCATATACTTTTACTTTACTCAAACAA
GTCCCTATCCTTTGATTTGTAGTTTAGCCCTTATTTTAATAAAAAAATATTAACATATGGAGTGTTTGGAAGTGAAGGAAGAAATG
CGCAGCATGTTTCAGAGAGTTCAACAAAATGGAGCATTTGGTTGATCACATGAGAACTTCATATCATTCACTTCATGAACCTACC
TGTGTACTTTGTAATAAACACTGCAGGTCCTTTGATTCTCTAAGGGAACATCTTATAGGTCCTTTACCGAAACAAGAATGCAATAA
ATTATTCAAAATCCTAGGATGCAAATTCTGCTTATCGATTCTCGAAAGCCCTCACGCTCTTAAGCTTCACCAACACCGATGCCGC
TTCTCCGGAGTAAATTATGGAACAATGTCTCGACCGGCGAATAAGTCTACTACTGTTGTGGACAATGGGTTTTCGTCGCATGTG
GTTGCACTGGCTTGCCAAATGGTGGATGGTGGTGGGAACAATGAGTCAATGGATGGTTGTGCTAGGGTTTGCATGGTTGACGA
ATACGAGAATATCATCTTCCATGTTTATGTGAAACCACCTATTTCAGTTCCAAATTATAGGTATGAAAACAGTGGCATTGGAGGC
GAACATTTGAGAGATGGAATGCCATTGAAACAAGTGCAAAGAAGGATCGAAGAGTTTCTTTGCAATGGAGAAGCAATGTGGAAA
ATTCGATCACCTAAAGCTGGAAAAGCTAGGATTCTTGTGGGTCATCATCTTCATCCTCTTCTTCAATCCCTGCATTTACAATATCC
ATCATTTATGATAAGGGATACTGCAGCATATCCACCTTTAATGAAAACAAACAAACTTAGCAACTCACTCAAGTACTTGACTCAAA
CATATTTGGGATATGATATACAAGCTGGCGTTCAAGACCCTTATGAAGACTGTGTTGCAACAATGAGGCTTTACCTGAGGATGA
GGAACCAAGTTCACCAGAGAGAAGACTATCCACAGGCTTCTGACCCTCGAAATCGGAACAACTTTGCACCATCAAGGCAAAGC
GAGCTTGAGAGAATGTCTCCGGAAGCAATGCTGGCTATCTCGAGGTCTGATTACTACTGTTGGTGCTTAGATTCCATGTAA
SEQIDNO: 228
AAGAAATGCGCAGCATGTTTCAGAGAGTTCAACAAAATGGAGCATTTGGTTGATCACATGAGAACTTCATATCATTCACTTCATG
AACCTACCTGTGTACTTTGTAATAAACACTGCAGGTCCTTTGATTCTCTAAGGGAACATCTTATAGGTATGTCTTCAACACTGGTT
AGTCTATTTTGTTTTAATTTTTTTGGGGTCCTTGAGCCGTGAAATCTGTTCACCTCAATATCTGAAAAAGTTTAGATAAAAATATTA
GACTTGAATTAAAATATGAGCTATACTTAAGCTCCATTATCTAACACTCAAGCTTAACCTAGTTCAATCTGTTCTCGATTTTAAAAA
TCAGTTAAACTCTGCTATTAGTCTTTGTACTTTTTGCGAAAGTTGTGAATTTTGTCCTTGTACTTTAAATTGATCAATTTTAGTCCC
TTTACTTCTAAAATTTTAGGCTTGACCCAAACAATAACAGTTAAATTTGTTTGGTTAGATTCAACTACTAATCAAGTACTATGCATA
CAGTAGTAGCGTTAATCAATATTCTCTAATTAGATCATTCTATGTCTCTATACTTTTTCAAAACTTGGAATTTTAGTCTCCAGACAT
GATAGTCACTAATTCATTAACCGCATTTTTAGTGAGTAATGTATGGAAATAATAAGCTGACATAGCATTATATACTCAACTTAATG
AACTTAACAATTATTGTTTAGTGAAAACTAAAATTTTAAAATTCGAAAAGTATAAGAACTAAAAATACTAAATTAAAATACAAGGAC
TAATAGTAAAATTTAACCTTATAAATTTAAAAAAATGAAAGTATATAAAAATACTAAATATTAAATATAATTTTTTTTAAAATTGATAG
ATTTAAATTAATTTGGGTTAATCATTAACAAATATGAATGAGTTTAGATAAAATTGATATTTTTATTTCAAATTAGACCTATTCGAAA
TCCAAACTACCCGCATATAGTCTCACCTAAAATTTGATAGGGTTAAGACAAAAATAGTAGGCATCAAAAAATGAACTTGGCATTG
GTATGAATCTTGATTTCAATGCAAGAGGATCTGAGTTCGAGCATATATGATTTAAAACCTATGATTTGATAAATTGTAGGTCCTTT
ACCGAAACAAGAATGCAATAAATTATTCAAAATCCTAGGATGCAAATTCTGCTTATCGATTCTCGAAAGCCCTCACGCTCTTAAG
CTTCACCAACACCGATGCCGCTTCTCCGGAGTAAATTATGTATTCATCTAATTTAGCTTTGAAAAAAAATTATTTCAAACTACAAT
GCTGAAATTTAGCTTAATTAAAATGCAGTTTTGGTGGACGTCTTATTACAGGGAACAATGTCTCGACCGGCGAATAAGTCTACTA
CTGTTGTGGACAATGGGTTTTCGTCGCATGTGGTTGCACTGGCTTGCCAAATGGTGGATGGTGGTGGGAACAATGAGTCAATG
GATGGTTGTGCTAGGGTTTGCATGGTTGACGAATACGAGAATATCATCTTCCATGTTTATGTGAAACCACCTATTTCAGTTCCAA
ATTATAGGTACCAACCACACAATTCGTGATCTTATAAAATTATGTCAATTACTATTTACGTGTGATTTTTTTATATAATATATTTTAG
TTTTTTTATTAATTTAAAATTAAAATATTTATTTTTATTATAATCAATTAAAGATTAAATAAAAAAAATTTCAATCAGCCAACAAACTC
TTGACCTAGTGGCTAGAACATTATGTTGTAGGCATGAGAACTTAAATTTTATCTTCAGAGTTTTAGTTGCATATCTCAATATCGCT
TTGACCCAAACGGTACTACACTGAAACTGTAAAATAAATAATGGGTACAGGACTTACCTTTTTAAAAATAGTAAAGTTGGAAGTAT
TTTTTTTAACTCAATAATAAAATTGAATGGAATTCAATAGAAATCAAATTCAACATTATAATTTAATTGACTTTTTATTTTCTCGAATT
TGTTAGCTCTAATGTCTAATGAAAGGAAATATAAAACAATAATTCACATAATTGAACTACGTGTTTTTTTTAGGTATGAAAACAGTG
GCATTGGAGGCGAACATTTGAGAGATGGAATGCCATTGAAACAAGTGCAAAGAAGGATCGAAGAGTTTCTTTGCAATGGAGAAG
CAATGTGGAAAATTCGATCACCTAAAGCTGGAAAAGCTAGGATTCTTGTGGGTCATCATCTTCATCCTCTTCTTCAATCCCTGCA
TTTACAATATCCATCATTTATGATAAGGTAAAGTTTACCATCACATTCATTACAATATACAAAATTTATATGATGTGATTTTATTATT
GCCTATAGGGATACTGCAGCATATCCACCTTTAATGAAAACAAACAAACTTAGCAACTCACTCAAGTACTTGACTCAAACATATTT
GGGGTAAGTGTATATATATTGTCCCTCAATCCCTATGATCATATCATTGATTGTTGACCCTTGTGTATATATGTATATATATCAGAT
ATGATATACAAGCTGGCGTTCAAGACCCTTATGAAGACTGTGTTGCAACAATGAGGCTTTACCTGAGGATGAGGAACCAAGTTC
ACCAGAGAGAAGACTATCCACAGGCTTCTGACCCTCGAAATCGGAACAACTTTGCACCATCAAGGCAAAGCGAGCTTGAGAGA
ATGTCTCCGGAAGCAATGCTGGCTATCTCGAGGTCTGATTACTACTGTTGGTGCTTAGATTCCATGTAA
SEQIDNO: 229
MDSRRESAETLRNKCSACFRQYNKMEHLVEHMKVSYHSVHEPKCGACRKHCRSFESLREHLIGPLPKAECARVFSARGCSICLNIFD
SPATARYHRHTCQYSRAAPMPKGGAGGRAVAMACKMVGGGSDGSVDLCARVCLVGEDENIIFQTYVKPTAPVTNYRYEVTGIRPEY
LRDAMPLKLVQRRIQDILCNGEPLWKIRPRSYGRARILVGHIVDHDLERLGLEYPAFMIRDTAKYPPLMKTTKLSNTLKYLAQAYLGYD
VHTGIQDPYEDCVAAMRLYIRMRSQAHPRDYATGSGEVQNNYPAWRQRELERMSPEELLALSGSDYYCWCLDP
SEQIDNO: 230
ATTTGAGAGATCTTGATCGACCTGCAGGAAGAATCTCCAGGAGCGCGATGGACAGCAGAAGGGAGTCCGCGGAGACCTTGAG
GAACAAGTGCTCGGCCTGCTTCCGGCAGTACAACAAGATGGAGCACCTTGTGGAGCACATGAAGGTGTCGTACCACTCGGTCC
ACGAGCCCAAGTGCGGCGCCTGCAGGAAGCACTGCCGCTCCTTCGAGTCCCTCAGGGAGCACCTCATCGGGCCGTTGCCCAA
GGCGGAATGTGCGCGTGTCTTCAGCGCCCGGGGCTGCAGCATCTGCCTCAACATCTTCGACAGCCCCGCCACCGCCAGATAT
CACCGTCACACCTGCCAGTACTCCCGCGCTGCTCCGATGCCAAAGGGTGGCGCAGGTGGACGCGCGGTCGCCATGGCCTGC
AAGATGGTCGGGGGAGGGAGCGACGGATCGGTGGACCTCTGCGCGAGAGTGTGCCTTGTTGGAGAAGATGAGAACATCATCT
TCCAGACCTATGTAAAACCCACGGCGCCCGTCACAAACTACAGGTATGAGGTGACTGGGATAAGGCCCGAGTACCTGCGGGA
CGCAATGCCACTGAAACTTGTGCAGAGGAGGATCCAGGACATCCTGTGCAACGGGGAGCCGCTGTGGAAGATACGGCCGAGG
AGCTATGGAAGGGCAAGGATCCTCGTCGGACATATCGTGGACCATGACCTTGAGCGCCTAGGTTTGGAGTACCCGGCATTCAT
GATCAGGGACACCGCAAAGTACCCGCCCCTGATGAAAACCACCAAGCTGAGCAACACCCTGAAGTACCTCGCACAAGCATATC
TTGGATATGATGTCCATACTGGCATTCAGGATCCATATGAGGATTGCGTCGCGGCGATGCGACTATATATCAGGATGAGATCAC
AAGCTCACCCGCGAGACTACGCCACCGGTTCAGGGGAGGTGCAGAATAACTATCCGGCCTGGAGGCAGAGGGAGCTGGAGA
GGATGAGCCCGGAAGAACTCCTGGCACTTTCAGGATCAGACTACTACTGCTGGTGCCTGGACCCCTAGACGGATGAGCTGAAG
AGAACAAGGCCCGGCCGGCCGACCGATGCTGGTTCGGTCATACTCCATCCGTAGTAATAAGGACTACGTATGTCTAATTATCTT
TGGACTTATATATGTATGGTGGGATTGAGTATAATATTGGTGATCAAGAGTTGTATGAGTCGTATTTAAGAGCAATGATATATAGT
AAGA
SEQIDNO: 231
ATTTGAGAGATCTTGATCGACCTGCAGGAAGAATCTCCAGGAGCGCGATGGACAGCAGAAGGGAGTCCGCGGAGACCTTGAG
GTATGAAGACGTTGAGAAACGCCATTTTTCCTTACCATAGCTAGTGACCTGCTAACATACAGCAGTAGCCGGTAGGGTTGCCTA
GCTCGAAGGAAGGGTGCCAGGAGAGATGATGGACGCTTGCCTGACTGAAGCATGTGTGTGTGTGTACCAGGAACAAGTGCTC
GGCCTGCTTCCGGCAGTACAACAAGATGGAGCACCTTGTGGAGCACATGAAGGTGTCGTACCACTCGGTCCACGAGCCCAAG
TGCGGCGCCTGCAGGAAGCACTGCCGCTCCTTCGAGTCCCTCAGGGAGCACCTCATCGGTAAGCTACCAGCAAACTCAACCA
CTACCATATCTCTAGCTATGGCATCCTCCGTCTATGTCTTGGGCTGAACAGCTGAACTGAACACGCCGCTCCTTGTGATCCTGG
CAATGAAGGGCCGTTGCCCAAGGCGGAATGTGCGCGTGTCTTCAGCGCCCGGGGCTGCAGCATCTGCCTCAACATCTTCGAC
AGCCCCGCCACCGCCAGATATCACCGTCACACCTGCCAGTACTCCCGCGCTGCTCCGGTAATTAGTGTTCTCCTTCTCCACTC
CCTTCAGTCGCCGCCATCGAGCTCTACGTGGCTTGGATGCTAGAGCTCCCGGCCAACTTTTTACAGAAATGTTGTTGGGAGAAC
AGAAATCAGAATCTGATGTTTCTCTTCTTTTTTCTCAGATGCCAAAGGGTGGCGCAGGTGGACGCGCGGTCGCCATGGCCTGCA
AGATGGTCGGGGGAGGGAGCGACGGATCGGTGGACCTCTGCGCGAGAGTGTGCCTTGTTGGAGAAGATGAGAACATCATCTT
CCAGACCTATGTAAAACCCACGGCGCCCGTCACAAACTACAGGTAGCCTACGTACCTTGGGACTCACTACTGCTCTTGCTTACT
AGCTTGACAAGATCAACTAGATGGATTGTTTTCTGAAGCGCAAAAGATCAAGCTGACCATTTGGCTCTTCAGGTATGAGGTGAC
TGGGATAAGGCCCGAGTACCTGCGGGACGCAATGCCACTGAAACTTGTGCAGAGGAGGATCCAGGACATCCTGTGCAACGGG
GAGCCGCTGTGGAAGATACGGCCGAGGAGCTATGGAAGGGCAAGGATCCTCGTCGGACATATCGTGGACCATGACCTTGAGC
GCCTAGGTTTGGAGTACCCGGCATTCATGATCAGGTACAGATCTTTGCTCCGCGAGCTACAGCTTGTTCCTACTATCTCTTCGT
GAATTCCCTCGAAATTTACTACTATTTTCTTAACACAATTTATCTATGGACGTGCAGGGACACCGCAAAGTACCCGCCCCTGATG
AAAACCACCAAGCTGAGCAACACCCTGAAGTACCTCGCACAAGCATATCTTGGGTATGTCACTTGATAACTGAGGATTACAACA
AACTCTCAGAATTTTTCTGGTATTTTGCATAATCCTCAAGAGGTTGTCCTTCTATCGCGTTGTAGATATGATGTCCATACTGGCAT
TCAGGATCCATATGAGGATTGCGTCGCGGCGATGCGACTATATATCAGGATGAGATCACAAGCTCACCCGCGAGACTACGCCA
CCGGTTCAGGGGAGGTGCAGAATAACTATCCGGCCTGGAGGCAGAGGGAGCTGGAGAGGATGAGCCCGGAAGAACTCCTGG
CACTTTCAGGATCAGACTACTACTGCTGGTGCCTGGACCCCTAGACGGATGAGCTGAAGAGAACAAGGCCCGGCCGGCCGAC
CGATGCTGGTTCGGTCATACTCCATCCGTAGTAATAAGGACTACGTATGTCTAATTATCTTTGGACTTATATATGTATGGTGGGA
TTGAGTATAATATTGGTGATCAAGAGTTGTATGAGTCGTATTTAAGAGCAATGATATATAGTAAGA
SEQIDNO: 232
MDSSSDAHSRNKCAACYRQFNRMEHLVEHMRASHHSPHEPRCGVCGKHCRSLDALRDHLGFGASLPPKPACARAFAAQGCPLCL
AVFPSAGSLRAHSPTCHLSRAPVPSMPMPRTPVGGAVALGCKMVGAGNDGTLDVCARVCVIDEHEAIVYEKFVRPLLPVTHYRYETT
GIRPEHLRDALAVKMVQRQVEDILLNGEQPWKVRSSRGAARLLVGHGLEHDLDALGMDYPAYLKRDTAVYPPLMKTSARLMSNSLR
FLTQSCLGYDIQTGHQHPYEDCVAAMRLYKKMRAMRHGRPNNVGDGDGCAAKPFPTWRQRELERMSPEELLSMSKPDYHCWCLD
N
SEQIDNO: 233
ATCATGGATAGCTCTTCAGATGCCCACAGCCGGAACAAGTGTGCGGCGTGCTACCGTCAGTTCAACAGGATGGAGCACCTGGT
GGAGCACATGCGCGCGTCGCACCACTCGCCACACGAGCCGCGCTGCGGCGTCTGCGGGAAGCACTGCCGCTCCCTCGACGC
CCTCCGTGACCACCTCGGCTTCGGCGCCTCCCTCCCCCCAAAGCCCGCCTGCGCCAGGGCCTTCGCCGCCCAGGGCTGCCC
GCTCTGCCTCGCCGTCTTCCCTAGCGCCGGCTCCCTCCGCGCCCACAGCCCCACCTGTCACCTCTCTCGTGCTCCGGTTCCCT
CGATGCCTATGCCAAGAACGCCCGTCGGCGGTGCGGTGGCGCTGGGGTGCAAAATGGTGGGCGCCGGCAACGACGGGACG
CTGGACGTGTGCGCGCGCGTCTGCGTCATCGACGAGCACGAGGCCATCGTCTACGAGAAGTTCGTGAGGCCGCTCCTCCCGG
TGACGCACTACCGGTACGAGACCACGGGGATCCGCCCCGAGCACCTTCGGGACGCGCTGGCGGTGAAGATGGTCCAGCGGC
AGGTGGAGGACATCCTCCTCAACGGCGAGCAGCCATGGAAGGTCCGGTCCTCCCGCGGCGCGGCCAGGCTCCTCGTCGGGC
ACGGCCTGGAGCACGATCTCGACGCGCTGGGCATGGACTATCCGGCGTACCTGAAGCGGGACACGGCGGTATACCCGCCGC
TGATGAAGACGAGCGCCAGGCTGATGAGCAACTCGCTCCGGTTCCTCACTCAGAGCTGCCTCGGCTATGACATCCAGACGGG
CCACCAGCATCCCTACGAGGACTGCGTGGCGGCCATGCGGCTGTACAAGAAGATGCGCGCGATGAGGCACGGCCGGCCCAA
CAACGTCGGAGACGGCGATGGGTGCGCCGCGAAGCCATTCCCGACGTGGAGGCAGCGGGAGCTGGAGCGCATGTCGCCGG
AGGAGCTCCTCAGCATGTCCAAGCCCGACTACCACTGCTGGTGCCTCGACAACTAGCTAGCCACGGCTTGCCTACAGAATTCG
ATTCTTCGCTCTTTACATACTTATATACTAGCACCGTACGTTGCAACGAAAGAAAAAATACGCATAATCCAAACTAAATAACTA
SEQIDNO: 234
ATCATGGATAGCTCTTCAGATGCCCACAGGTGCACACACTGTCCACTGCTCCGTTTGAGTTGTTACACATTTTGCAGTGGAAGA
ACTCTACTGTATACTCTGCAGTATTTCTCGAAAGTTTTGTGAGATCATGCCACGAAATTAAGTTATCTGCACACTTTGTTTGTTTC
TACATTACGTGATAAGTTGAGGTTGATTCTTAAGAATCTTATAATTGTTTTCTTAAGAATCTAGTATTCGTCGTCTGCATGCAGCC
GGAACAAGTGTGCGGCGTGCTACCGTCAGTTCAACAGGATGGAGCACCTGGTGGAGCACATGCGCGCGTCGCACCACTCGCC
ACACGAGCCGCGCTGCGGCGTCTGCGGGAAGCACTGCCGCTCCCTCGACGCCCTCCGTGACCACCTCGGCTTCGGCGCCTC
CCTCCCCCCAAAGCCCGCCTGCGCCAGGGCCTTCGCCGCCCAGGGCTGCCCGCTCTGCCTCGCCGTCTTCCCTAGCGCCGG
CTCCCTCCGCGCCCACAGCCCCACCTGTCACCTCTCTCGTGCTCCGGTTCCCTCGATGCCTATGCCAAGAACGCCCGTCGGC
GGTGCGGTGGCGCTGGGGTGCAAAATGGTGGGCGCCGGCAACGACGGGACGCTGGACGTGTGCGCGCGCGTCTGCGTCAT
CGACGAGCACGAGGCCATCGTCTACGAGAAGTTCGTGAGGCCGCTCCTCCCGGTGACGCACTACCGGTACGAGACCACGGG
GATCCGCCCCGAGCACCTTCGGGACGCGCTGGCGGTGAAGATGGTCCAGCGGCAGGTGGAGGACATCCTCCTCAACGGCGA
GCAGCCATGGAAGGTCCGGTCCTCCCGCGGCGCGGCCAGGCTCCTCGTCGGGCACGGCCTGGAGCACGATCTCGACGCGCT
GGGCATGGACTATCCGGCGTACCTGAAGCGGGACACGGCGGTATACCCGCCGCTGATGAAGACGAGCGCCAGGCTGATGAG
CAACTCGCTCCGGTTCCTCACTCAGAGCTGCCTCGGCTATGACATCCAGACGGGCCACCAGCATCCCTACGAGGACTGCGTG
GCGGCCATGCGGCTGTACAAGAAGATGCGCGCGATGAGGCACGGCCGGCCCAACAACGTCGGAGACGGCGATGGGTGCGCC
GCGAAGCCATTCCCGACGTGGAGGCAGCGGGAGCTGGAGCGCATGTCGCCGGAGGAGCTCCTCAGCATGTCCAAGCCCGAC
TACCACTGCTGGTGCCTCGACAACTAGCTAGCCACGGCTTGCCTACAGAATTCGATTCTTCGCTCTTTACATACTTATATACTAG
CACCGTACGTTGCAACGAAAGAAAAAATACGCATAATCCAAACTAAATAACTA
SEQIDNO: 235
MMMGRRESSETLRNKCAACYRQYNRMEHLVEHMKVNYHSVHEPRCGVCGKHCRSFESLREHLIGPLPKVECARVFSVRGCSICLNI
FDSNAAVRYHRAACQYTRAAPMPRGGITGRAVALACKMVGGGSDGSVDLCARVCLIGEDENIIFQTYVKPTAPVTNYRYEVTGVRPE
YLRDAMPLKVAQRRIQEILCNGEPLWKLRPRSYGRAKILVGHGLEHDLERLGLEYPAFMIRDTAKYPPLLKTSKLSNSLKYLTQAYLGY
DIQTGIQDPYEDCVAAMRLYIRMRSQAHPRDYNSGSGEAQNNYPAWRQRELERMSPEELLALSASDYYCWCLDY
SEQIDNO: 236
CTCGAGCTCGATCTCTTCTCAGGGACCTTTGACCCAAGTGTTGGAATCTGAGAATGATGATGGGCAGGAGGGAGTCCTCGGAG
ACCTTGAGGAACAAATGCGCGGCCTGCTACAGGCAGTACAACAGGATGGAGCACCTGGTGGAGCACATGAAGGTGAACTACC
ACTCGGTGCACGAGCCCAGGTGCGGCGTCTGCGGCAAGCACTGCCGCTCCTTCGAGTCGCTCAGGGAGCATCTGATCGGGC
CATTGCCCAAGGTGGAGTGCGCGCGGGTTTTCAGCGTCCGCGGCTGCAGCATCTGCCTCAACATCTTCGACAGCAACGCCGC
CGTCAGATACCACCGTGCAGCCTGCCAATACACTCGTGCTGCTCCGATGCCCAGGGGTGGCATAACTGGCCGTGCGGTTGCT
CTGGCTTGTAAAATGGTTGGAGGAGGGAGTGACGGCTCAGTGGACCTTTGTGCAAGGGTGTGCCTCATTGGAGAAGACGAGA
ATATCATCTTCCAGACCTATGTCAAACCTACAGCTCCAGTCACCAACTACAGGTATGAAGTAACTGGGGTAAGGCCAGAGTACT
TGAGGGACGCAATGCCACTGAAGGTTGCACAGAGGAGGATCCAGGAGATCTTGTGCAACGGGGAGCCGCTGTGGAAGTTACG
CCCAAGGAGTTATGGAAGGGCAAAGATCCTGGTTGGCCATGGCCTGGAACATGATCTTGAGCGCCTAGGGTTGGAGTACCCC
GCATTCATGATCAGGGACACTGCAAAATACCCGCCTCTGTTGAAGACTAGCAAACTGAGTAACTCCCTGAAGTATCTTACACAA
GCATATCTTGGGTATGATATTCAAACAGGCATTCAGGATCCCTATGAGGACTGTGTGGCAGCAATGAGGCTGTACATCAGGATG
AGATCACAGGCTCACCCGAGAGATTATAACTCCGGTTCAGGTGAGGCCCAGAACAACTACCCAGCCTGGAGGCAGAGAGAACT
GGAGAGGATGAGCCCAGAAGAACTGCTGGCACTTTCAGCATCAGACTATTATTGCTGGTGCCTGGATTACTAAATTGATCGGCT
ATAAAGCGAATAAGGCAGGCCAACATTGTTGGTCCATTCATATTCCGTCTACGGCAATAATTTTAGATGTACAAATAGCTTGCGG
CTTGACTATACTGGGGTTAATTATAACATATATACATGTAACGCTTTCATATAAATATTGGTGATTTAGAGCTGTAAGAAGTGATA
TTATCTTAATGATATTATATGGCAAGAGGGTTTTTTTACCTTGCTTTATGAATCCAGAAAATGAGAAAACCAGAAGTACATCAACT
ATACGCCAAAGGCAGATAAAAAGGACATTTATCTACATGTAACGAATGGACGATTGTACATTCTGTGCAGCGTGTCAGACTATCT
CAGTGCTATACAAGTATATCTACATTAGCCTGCAGTAGACCATACCTGTGCGAAATGAAGATTCAAGTTTCTGAGCTGTTGAAGA
AGAGCCTTACACTGGAGTCATTGAATTTCCGGCATTGCTTAGCTTCTGCGGGTGGATTCTTAGGTTTGGGCTCGTCCGGTTTTG
CCTGCTTGTCACCCTTGTCCTTGGATTGGCTCCTATTTAGCTTGATGAACTCAAGACACTGTCGAATGATTGTCAGCACCGTGAA
ATGTGTCTCAAAGTCATTCTTGATGACAGGTGGATGGTGGAGGTTGGGCCCATTGTTGCTCCAGGTGTAGTTCTCGATGAAGTA
AACACAATCAGTTCGGGCCCTGAACGAAACCTCCCGTAGTAGCTCTTCAGCCTGCCTAATTGATCTCATGGCCTTCTTAAAAGT
CAATACAACGATCAAATTAAGC
SEQIDNO: 237
CTCGAGCTCGATCTCTTCTCAGGGACCTTTGACCCAAGTGTTGGAATCTGAGAATGATGATGGGCAGGAGGGAGTCCTCGGAG
ACCTTGAGGTAAGATGATAAGAGATGCTATTCTTTCTTTCAGCTTTCAGCATTGATATGCGCGCTTCGATGAACATGCACTCCAG
GCTCAAGTTCATCCCACACTGCACCTAACCCTTATAGAGATGTAGGCATATATGCAGAAACCATCAAGGCACCTCAACAATCAAT
CTAAGTCAATCTAAGGCTTGAAACATATCCATTATATTTACTGTAACCACGTGAAACCAGCCAGATTGCGGTTGCAGAACTATAC
AAGCCGCTGCTCCGAGTAATCTGGAAGTGTAGCTAGATTAATCTATGCATCAAAGTAACTGAAGCATGTGCATGTATGTCTCCC
ATGATCAGGAACAAATGCGCGGCCTGCTACAGGCAGTACAACAGGATGGAGCACCTGGTGGAGCACATGAAGGTGAACTACC
ACTCGGTGCACGAGCCCAGGTGCGGCGTCTGCGGCAAGCACTGCCGCTCCTTCGAGTCGCTCAGGGAGCATCTGATCGGTAC
GCAGAAAACTGAGAACCAAGGACAGCAGCTCACAGCTGAACGATCGTGGTTTCTGACGCTGCCTCTTCTCAATCTTTAGGGCC
ATTGCCCAAGGTGGAGTGCGCGCGGGTTTTCAGCGTCCGCGGCTGCAGCATCTGCCTCAACATCTTCGACAGCAACGCCGCC
GTCAGATACCACCGTGCAGCCTGCCAATACACTCGTGCTGCTCCGGTAATGATTTACTGATTTCTACTCTATTCATTCATATTAC
CATATATTCAGTTCATTTCAGAACTCAGATGCATAGACATCCCTTTGTTGTGACTTTGGTTGATGCTAGATTCTAACATTTTTCTTC
CTTTCCATCTGGTCCCAGATGCCCAGGGGTGGCATAACTGGCCGTGCGGTTGCTCTGGCTTGTAAAATGGTTGGAGGAGGGAG
TGACGGCTCAGTGGACCTTTGTGCAAGGGTGTGCCTCATTGGAGAAGACGAGAATATCATCTTCCAGACCTATGTCAAACCTAC
AGCTCCAGTCACCAACTACAGGTAACATTTCATTCCATTACTTTTCCTGCATACCAGTAGTCTCCCATTTGTAATAGATGGTACAA
GAGAAATTATCAAACCAGCGAAAAAAACCAGAAAGAAAGCATTAGCGATCATTGAGTGGACACTATGCTGGAAACTGGACAGGA
GATTTGCATGGACCACTTTCTAAAATGAGACGAGCTGATTATTTGTGTGTTCAGGTATGAAGTAACTGGGGTAAGGCCAGAGTA
CTTGAGGGACGCAATGCCACTGAAGGTTGCACAGAGGAGGATCCAGGAGATCTTGTGCAACGGGGAGCCGCTGTGGAAGTTA
CGCCCAAGGAGTTATGGAAGGGCAAAGATCCTGGTTGGCCATGGCCTGGAACATGATCTTGAGCGCCTAGGGTTGGAGTACC
CCGCATTCATGATCAGGTGAGGAGAACTAGATAACTAAGTGGGTTGTAGTCTATCTGCTTTCTAACATATATAGATCTTGCTTAC
CTAGCTATGGTTTAGGAATCCAGAGTCAAAGTTGCTACGGTTTCTAACATTGACGCATGTGCAGGGACACTGCAAAATACCCGC
CTCTGTTGAAGACTAGCAAACTGAGTAACTCCCTGAAGTATCTTACACAAGCATATCTTGGGTATGTCACTAGCAGGCTATTCTA
AACAATCAGCCTACTACTTAGTATCTCCGTAATGCTAAATAGATTGTTTTTGCATCTTATTGTAGGTATGATATTCAAACAGGCATT
CAGGATCCCTATGAGGACTGTGTGGCAGCAATGAGGCTGTACATCAGGATGAGATCACAGGCTCACCCGAGAGATTATAACTC
CGGTTCAGGTGAGGCCCAGAACAACTACCCAGCCTGGAGGCAGAGAGAACTGGAGAGGATGAGCCCAGAAGAACTGCTGGCA
CTTTCAGCATCAGACTATTATTGCTGGTGCCTGGATTACTAAATTGATCGGCTATAAAGCGAATAAGGCAGGCCAACATTGTTGG
TCCATTCATATTCCGTCTACGGCAATAATTTTAGATGTACAAATAGCTTGCGGCTTGACTATACTGGGGTTAATTATAACATATAT
ACATGTAACGCTTTCATATAAATATTGGTGATTTAGAGCTGTAAGAAGTGATATTATCTTAATGATATTATATGGCAAGAGGGTTT
TTTTACCTTGCTTTATGAATCCAGAAAATGAGAAAACCAGAAGTACATCAACTATACGCCAAAGGCAGATAAAAAGGACATTTAT
CTACATGTAACGAATGGACGATTGTACATTCTGTGCAGCGTGTCAGACTATCTCAGTGCTATACAAGTATATCTACATTAGCCTG
CAGTAGACCATACCTGTGCGAAATGAAGATTCAAGTTTCTGAGCTGTTGAAGAAGAGCCTTACACTGGAGTCATTGAATTTCCG
GCATTGCTTAGCTTCTGCGGGTGGATTCTTAGGTTTGGGCTCGTCCGGTTTTGCCTGCTTGTCACCCTTGTCCTTGGATTGGCT
CCTATTTAGCTTGATGAACTCAAGACACTGTCGAATGATTGTCAGCACCGTGAAATGTGTCTCAAAGTCATTCTTGATGACAGGT
GGATGGTGGAGGTTGGGCCCATTGTTGCTCCAGGTGTAGTTCTCGATGAAGTAAACACAATCAGTTCGGGCCCTGAACGAAAC
CTCCCGTAGTAGCTCTTCAGCCTGCCTAATTGATCTCATGGCCTTCTTAAAAGTCAATACAACGATCAAATTAAGC
SEQIDNO: 238
MDSSSDAHGRHRCAACFRQFNKMEHLVEHMRAARHSAHEPRCGLCGKHCRSFEALRDHLGVGGSTLPKATACADAFAARGCAICL
RVTPHHRASCTLARTPRTPQAAAPPQGGGSGRALALGCKMVGAGSDGSLDVVARVCVVDEQENIVYEAFVKPLIPVTHYRYETTGIR
PENLRDGGGAVTVKAAQRRVQDLLLAGEQPWKVRTSRGRARLLVGHGLDHDLDALGMDYPAYLKRDTAAYPPLMKTSKLSNSLRFL
TRTYLGYEIQTGHQHPYEDCVAAMRLYRRMKEQGHTRRGGDADEPAASADQAFPAWRQRELERMTPEELLRLSTPDYRCWCLDD
SEQIDNO: 239
CCCGTGCGCAGCCTTCGCCGCGACAGCTCGACCGACACCAATCGACAGGTTCAGTGAGCTGTGCTAGTGACGAACTGACTGA
ACTGACTGACGATGGATAGCTCCTCTGATGCTCATGGGCGTCACAGGTGCGCGGCGTGCTTCCGGCAGTTCAACAAGATGGA
GCACCTGGTGGAGCACATGCGGGCGGCGCGGCACTCGGCGCACGAGCCCCGCTGCGGCCTCTGCGGCAAGCACTGCCGCT
CCTTCGAGGCGCTCCGCGATCACCTCGGCGTCGGCGGCTCCACGCTGCCCAAGGCCACCGCCTGCGCCGACGCCTTCGCCG
CGCGCGGCTGCGCCATCTGCCTCCGCGTCACGCCCCACCACCGCGCCTCCTGCACGCTCGCGCGCACCCCCAGGACGCCGC
AGGCGGCGGCGCCGCCCCAGGGAGGAGGAAGTGGGCGGGCGCTGGCGCTGGGCTGCAAGATGGTCGGTGCCGGCAGCGA
CGGCTCCCTGGACGTGGTGGCGCGGGTGTGCGTCGTCGACGAGCAGGAGAACATCGTGTACGAGGCCTTCGTGAAGCCGCT
CATCCCGGTGACGCACTACCGGTACGAGACGACGGGGATCCGGCCGGAGAACCTCCGCGACGGCGGCGGCGCCGTGACGG
TGAAGGCCGCGCAGCGGCGGGTGCAGGACCTGCTGCTCGCCGGCGAGCAGCCGTGGAAGGTGCGCACCAGCCGGGGCCGG
GCCAGGCTGCTGGTGGGCCATGGCCTGGACCACGACCTCGACGCGCTGGGGATGGACTACCCGGCCTACCTCAAGCGCGAC
ACGGCCGCGTACCCGCCGCTGATGAAGACGAGCAAGCTCAGCAACTCGCTCAGGTTCCTCACGCGCACCTACCTCGGGTACG
AGATCCAGACGGGGCACCAGCACCCCTACGAGGACTGCGTCGCCGCCATGCGCCTCTACCGCAGGATGAAGGAGCAGGGCC
ACACCAGGCGTGGCGGCGACGCCGACGAGCCGGCGGCCAGCGCGGACCAGGCGTTCCCGGCGTGGAGGCAGCGGGAGCT
GGAGCGCATGACGCCCGAGGAACTGCTCCGGCTCTCCACGCCGGACTACCGCTGCTGGTGCCTCGATGACTAG
SEQIDNO: 240
CCCGTGCGCAGCCTTCGCCGCGACAGCTCGACCGACACCAATCGACAGGTTCAGTGAGCTGTGCTAGTGACGAACTGACTGA
ACTGACTGACGATGGATAGCTCCTCTGATGCTCATGGGTGAGTTGCAACTTCTCTACTCTCTTCAGACCTCGCTGAAGGGTTCA
GCACTTCAGCATCACTTCAGTCTCTTTAGACGAAGGCAAGGCCCAAGGGGACATAACACTTGCATGCATGCTTTTGATGGCACA
ATTAGACAGCTTAAACATGCTTTAATTTGTAGTATAATATCTTATTCTAACCAACCCTTGAAAAATGTGATTGCGTTATGGCAGGC
GTCACAGGTGCGCGGCGTGCTTCCGGCAGTTCAACAAGATGGAGCACCTGGTGGAGCACATGCGGGGGGCGCGGCACTCGG
CGCACGAGCCCCGCTGCGGCCTCTGCGGCAAGCACTGCCGCTCCTTCGAGGCGCTCCGCGATCACCTCGGCGTCGGCGGCT
CCACGCTGCCCAAGGCCACCGCCTGCGCCGACGCCTTCGCCGCGCGCGGCTGCGCCATCTGCCTCCGCGTCACGCCCCACC
ACCGCGCCTCCTGCACGCTCGCGCGCACCCCCAGGACGCCGCAGGCGGCGGCGCCGCCCCAGGGAGGAGGAAGTGGGCG
GGCGCTGGCGCTGGGCTGCAAGATGGTCGGTGCCGGCAGCGACGGCTCCCTGGACGTGGTGGCGCGGGTGTGCGTCGTCG
ACGAGCAGGAGAACATCGTGTACGAGGCCTTCGTGAAGCCGCTCATCCCGGTGACGCACTACCGGTACGAGACGACGGGGAT
CCGGCCGGAGAACCTCCGCGACGGCGGCGGCGCCGTGACGGTGAAGGCCGCGCAGCGGGGGGTGCAGGACCTGCTGCTCG
CCGGCGAGCAGCCGTGGAAGGTGCGCACCAGCCGGGGCCGGGCCAGGCTGCTGGTGGGCCATGGCCTGGACCACGACCTC
GACGCGCTGGGGATGGACTACCCGGCCTACCTCAAGCGCGACACGGCCGCGTACCCGCCGCTGATGAAGACGAGCAAGCTC
AGCAACTCGCTCAGGTTCCTCACGCGCACCTACCTCGGGTACGAGATCCAGACGGGGCACCAGCACCCCTACGAGGACTGCG
TCGCCGCCATGCGCCTCTACCGCAGGATGAAGGAGCAGGGCCACACCAGGCGTGGCGGCGACGCCGACGAGCCGGCGGCC
AGCGCGGACCAGGCGTTCCCGGCGTGGAGGCAGCGGGAGCTGGAGCGCATGACGCCCGAGGAACTGCTCCGGCTCTCCAC
GCCGGACTACCGCTGCTGGTGCCTCGATGACTAG
SEQIDNO: 241
MVDSRRESSETLRNKCAACFRQYNRMEHLVEHMKVSYHSVHEPKCGVCRKHCRSFESLREHLIGPLPKVECARVFSVRGCSICLNIF
DSNATVRYHRAACQYSRAAPQMPRGGITGRAVALACKMVGGGSDGSMDLCAKVCLIGEDEHIIFQTYVKPILPVTNYRYEVTGIRPEY
LRDAMPLKVAQRRIQEILCNGEPLWKLRPRSYGRAKILVGHGLDHDLERLGLEYPAFMIRDTAKYPPLVKTSKLSNSLKYLTQAYLGY
DIHTGIQDPYEDCVAAMRLYIRMRSQAHPRDYNSGSGEAQNNYPAWRQRELERMSPEELLALSASDYYCWCLDY
SEQIDNO: 242
CGATCTTGGCCTCTCAGGGACCTTGACCGCCCAAGTCCCGAATTGTAGGAATCTGAGCGAGGGCTCATGGTGGACAGCAGGA
GGGAGTCCTCGGAGACCTTGAGGAACAAATGCGCAGCCTGCTTCAGGCAGTACAACAGGATGGAGCACCTGGTGGAGCACAT
GAAGGTCTCGTACCACTCGGTGCACGAGCCCAAGTGCGGCGTCTGCCGGAAGCACTGCCGCTCCTTCGAGTCGCTCAGGGAG
CATCTCATCGGGCCGTTGCCCAAGGTGGAGTGCGCGCGGGTCTTCAGCGTCCGCGGCTGCAGCATCTGCCTCAACATTTTCG
ACAGCAACGCCACCGTCAGATACCACCGTGCTGCCTGCCAATACTCTCGTGCTGCTCCGCAGATGCCCAGGGGTGGCATAACT
GGCCGTGCGGTTGCACTGGCTTGCAAAATGGTAGGGGGAGGGAGTGACGGCTCAATGGACCTTTGTGCAAAGGTGTGCCTCA
TTGGAGAAGATGAGCACATCATCTTCCAGACCTATGTCAAGCCTATACTTCCTGTAACCAACTACAGGTATGAAGTAACTGGGAT
AAGGCCAGAGTACTTGAGGGATGCAATGCCGCTTAAGGTTGCGCAGAGGAGAATCCAGGAAATCCTGTGCAACGGGGAGCCG
CTGTGGAAGTTACGCCCAAGGAGCTATGGGAGGGCAAAGATCCTTGTTGGTCATGGCCTGGACCATGACCTTGAGCGCCTAG
GGTTGGAGTACCCAGCATTCATGATTAGGGATACTGCGAAATACCCACCACTAGTGAAGACTAGCAAGCTGAGTAACTCCCTGA
AGTACCTTACACAAGCATACCTTGGGTATGATATACATACTGGCATTCAGGACCCCTACGAGGACTGCGTTGCAGCAATGAGGC
TGTACATCAGGATGAGATCACAAGCTCACCCGAGAGATTACAACTCCGGCTCTGGCGAGGCCCAGAACAACTACCCAGCCTGG
AGGCAGAGAGAGCTCGAGAGGATGAGCCCAGAAGAACTCCTGGCACTTTCAGCATCAGATTACTATTGCTGGTGCCTGGATTA
CTAAACTGATCGGCTATAAAGAAAATAAGGCAGGCCAACATTGTTGGTTCATCCATATTCCGTCTATGATAATAAATCTTTAGATG
TAGCTTGCGGCTTGACTATATATTTGGGTTAACTATGACGTATAAACAAAGGATTATGTATACATGTAATGATGCTTTCATATAAG
CTAGTATTGTTGTTTTAGATCTGTATAAGAAGTGATATTATGATTTATGAT
SEQIDNO: 243
CGATCTTGGCCTCTCAGGGACCTTGACCGCCCAAGTCCCGAATTGTAGGAATCTGAGCGAGGGCTCATGGTGGACAGCAGGA
GGGAGTCCTCGGAGACCTTGAGGTAAGATGATAAGAGACGATGCTATTCTTTCAGCTTTCATTGATATGCGTACTGCAAATGGC
CGTGCACTCATCAGACTGATTAAGTGCATGCACGCATGTCCATTTCTTGTTACTATGAGCCTGTCAGACTGCGGTTGCGAGAGC
TAGGCAGCTAGCGAGCTATGCTGTTCATGCATGAAACTGCTCGTGCAAGGATCATCAGCTAGTTTCAGTTCGCCTCCTGCATTC
GATCCTATATATGTTCCTGAAAGCTAGAATCCATGCATCATGATCACAAACACATATATACGAATGAATTCCCTCATGCATGTTAT
TTATGCCCCCCATGAATCATGATCATCAGGAACAAATGCGCAGCCTGCTTCAGGCAGTACAACAGGATGGAGCACCTGGTGGA
GCACATGAAGGTCTCGTACCACTCGGTGCACGAGCCCAAGTGCGGCGTCTGCCGGAAGCACTGCCGCTCCTTCGAGTCGCTC
AGGGAGCATCTCATCGGTATGCACAAATAAAGCTTTTGCTGACAAGAAAAAGACGGCAGCTTAATTAACTACATACAGCTGAACT
CAATTCTGACGACGCTTCTTGTTCTTCTTCTCCATCTTCCTTCAGGGCCGTTGCCCAAGGTGGAGTGCGCGCGGGTCTTCAGCG
TCCGCGGCTGCAGCATCTGCCTCAACATTTTCGACAGCAACGCCACCGTCAGATACCACCGTGCTGCCTGCCAATACTCTCGT
GCTGCTCCGGTAATCATATCGATCCATCTGTTGCTCTCTACCCTATTCATTATTCATCATATGCCGTATATATATTCAGTTGATCTT
CCCTCGTGAAGGGCTGAAGGCTTTGGTTGATATATACTAGATTCTAATGTTTTTTTTTCCACCCTCCTATATATGTGGTCCCAGCA
GATGCCCAGGGGTGGCATAACTGGCCGTGCGGTTGCACTGGCTTGCAAAATGGTAGGGGGAGGGAGTGACGGCTCAATGGA
CCTTTGTGCAAAGGTGTGCCTCATTGGAGAAGATGAGCACATCATCTTCCAGACCTATGTCAAGCCTATACTTCCTGTAACCAAC
TACAGGTAATCCTACTTCTCATGCAGTTGTCATGCATCATGTTACTAGTCTACTAGCAACTCTAGCTAGTCGATAGTTGAGGAGA
AATTGTGAGACCAGCAAAATCAGAAAGAAAGAATCGGCAATCTTTCAGGGTGTACCACTATATATATGCTTGAAACTTGAAACTC
TCGAGAGGACAGGGAAGATATCTGGATGGACTAATTTTGTGACGACACGAACTGATGACTATATGTAACATGTTTTTCAGGTATG
AAGTAACTGGGATAAGGCCAGAGTACTTGAGGGATGCAATGCCGCTTAAGGTTGCGCAGAGGAGAATCCAGGAAATCCTGTGC
AACGGGGAGCCGCTGTGGAAGTTACGCCCAAGGAGCTATGGGAGGGCAAAGATCCTTGTTGGTCATGGCCTGGACCATGACC
TTGAGCGCCTAGGGTTGGAGTACCCAGCATTCATGATTAGGTGAGAAGCAGAAAAAAAAATGTCACTGAATTTCTCTTTTCTTCA
TTCCTAACATTTATAGATCTTTGCTTGCTGAGCTATGGTTCAGGAATCAGCTACCACAGTTGTTTTGCCCTTTTTGACAGAAAAAA
TTGTTATGCATATGCAGGGATACTGCGAAATACCCACCACTAGTGAAGACTAGCAAGCTGAGTAACTCCCTGAAGTACCTTACA
CAAGCATACCTTGGGTATGTCACTTCTTGAATGATCTATGCAGTCAGCCTACTATTTCATATCTGCGTAATGATGCTAAGGAGAT
TGTATCCTCTTGTAGGTATGATATACATACTGGCATTCAGGACCCCTACGAGGACTGCGTTGCAGCAATGAGGCTGTACATCAG
GATGAGATCACAAGCTCACCCGAGAGATTACAACTCCGGCTCTGGCGAGGCCCAGAACAACTACCCAGCCTGGAGGCAGAGA
GAGCTCGAGAGGATGAGCCCAGAAGAACTCCTGGCACTTTCAGCATCAGATTACTATTGCTGGTGCCTGGATTACTAAACTGAT
CGGCTATAAAGAAAATAAGGCAGGCCAACATTGTTGGTTCATCCATATTCCGTCTATGATAATAAATCTTTAGATGTAGCTTGCG
GCTTGACTATATATTTGGGTTAACTATGACGTATAAACAAAGGATTATGTATACATGTAATGATGCTTTCATATAAGCTAGTATTGT
TGTTTTAGATCTGTATAAGAAGTGATATTATGATTTATGAT
SEQIDNO: 244
MDSASDAHGRHKCAACFRQFNKMEHLVEHMRAARHSSHEPRCDICRKHCRSFEALRDHLGVGGSTLPKAAYCADAFAARGCTICL
RVLASAAALGAHSAACRLSRIPPPMMSRALQHHQLPAATQPHGGGGEGGALALGCKMVGAGSDGSLDVCARVCVIDEQENILFEAF
VKPLLPVTHYRYETTGIRPEHLRDGASTTVKNAQRRVEELLLNGEQPWKVRTSRGRARLLVGHGLDHDLDALGMDYPGYLKRDTAT
YPPLMKTSKLSNSLRFLTHKYLGYDIQTGHQHPFEDCVAAMRLYRRMRGQQQHPRGGGGDADAPAAATADQAFPAWRQRELERM
TPEELLRLSTPDYRCWCLDA
SEQIDNO: 245
CACACACGGCAGAAAGCTTTGGTAAAGTGAGTGAGCGAGCGAGCAGCAGTGGCTGTCGCTCGCGATGATCATGAGTGTTCGAT
CATCACATAGTGACACAACAATGGCGACAGAGGATCTAGTTCCATCCTCCGGCCTAGTATAAATAGGCACCCGTGTTAAGGCTC
CTTGCTGACAGATCATCACATCGGTCGACCAACCGAGCAGTTCAGTGAACCGTAGATCGACGTGACGATGGATAGCGCCTCAG
ATGCTCATGGGCGTCACAAGTGCGCGGCATGCTTCCGGCAGTTCAACAAGATGGAGCACCTGGTGGAGCACATGCGGGCGGC
GCGGCACTCGTCGCACGAGCCCCGCTGCGACATCTGCCGCAAGCACTGCCGCTCCTTCGAGGCGCTCAGGGACCACCTCGG
CGTCGGCGGCTCCACGCTGCCCAAGGCCGCCTACTGCGCCGACGCCTTCGCCGCGCGCGGCTGCACCATCTGCCTCCGCGT
CCTCGCCAGCGCCGCCGCGCTCGGAGCCCACAGCGCGGCGTGCCGGCTCTCGCGCATCCCACCGCCGATGATGTCGAGGGC
GCTGCAGCATCACCAGCTTCCAGCAGCAACGCAGCCGCATGGAGGAGGAGGAGAAGGAGGCGCGCTGGCGCTGGGCTGCAA
GATGGTCGGCGCCGGGAGCGACGGGTCCCTGGACGTGTGCGCGCGGGTGTGCGTCATCGACGAGCAGGAGAACATCCTGTT
CGAGGCCTTCGTGAAGCCGCTCCTCCCGGTGACGCACTACCGGTACGAGACGACGGGGATCCGGCCGGAGCACCTCCGCGA
CGGCGCGAGCACGACGGTGAAGAACGCGCAGCGGCGGGTGGAGGAGCTGCTGCTCAACGGCGAGCAGCCGTGGAAGGTGC
GCACGAGCCGGGGCAGGGCGAGGCTGCTGGTCGGGCACGGCCTGGACCACGACCTCGACGCGCTGGGCATGGACTACCCG
GGCTACCTCAAGCGCGACACGGCCACGTACCCGCCGCTGATGAAGACGAGCAAGCTCAGCAACTCGCTCAGGTTCCTGACGC
ACAAGTACCTCGGGTACGACATCCAGACGGGGCACCAGCACCCCTTCGAGGACTGCGTCGCCGCCATGCGCCTCTACCGCAG
GATGCGCGGGCAGCAGCAGCACCCCAGGGGGGGGGGGGCGACGCCGACGCACCGGCGGCGGCGACCGCGGACCAGGCG
TTCCCGGCGTGGAGGCAGCGGGAGCTCGAGCGCATGACGCCTGAGGAGCTCCTTCGGCTCTCCACGCCGGACTACCGCTGC
TGGTGCCTCGATGCGTAG
SEQIDNO: 246
CACACACGGCAGAAAGCTTTGGTAAAGTGAGTGAGCGAGCGAGCAGCAGTGGCTGTCGCTCGCGATGATCATGAGTGTTCGAT
CATCACATAGTGACACAACAATGGCGACAGAGGATCTAGTTCCATCCTCCGGCCTAGTATAAATAGGCACCCGTGTTAAGGCTC
CTTGCTGACAGATCATCACATCGGTCGACCAACCGAGCAGTTCAGTGAACCGTAGATCGACGTGACGATGGATAGCGCCTCAG
ATGCTCATGGGTGAGTTGATGCTGCTCTGAGTCTTGAGGTCACAATGTATGCTTTAAGCCTCAAACATATATATATTCCTTTGAG
TTTGAGGTAAATGCATGCTATACCTACCATGCTTGGGACACCTGGATTTTGTGAACCACTTCTATCACAAAAATATAAGGCCATC
AGTGTTTGTTCGAAATCCTTTTTTGGTTTCACTACCAATGTGTGGCTCTAATTTTATATGAGTTGACAGCATAAAATTTGCATTACT
AGAACCATTGTGGACCATCTATTGCTAGTTAAAGTTTCTTTTTGAAACGACCGGCTAGGAGTTTTGTCTGGCATGTATTAATAGA
GAAGAAGATTGGGTTTAAATTTGAAATAGAAGTTCAAAAAGAAGGTTGGGTTGGGGGTGGGGGGCGTAATGAAGTTATTATATT
ATAGAGGTCCAGTAGAGGGCCACTCTCTCAGAGATGATCTCCTTGAGTCTCTCGGCTCCTGCAAGGCTGCAAGGCATCATCTG
ATGCTCACTTCCTCCTTGATTTTCACAATTAGGCTTCCCAAAGGGGAATCGTGACTTTGAAAAAACTAGCATTGTGTCACTTCCA
GAGCTCCCAAGCTGCTACTACGATTGTTCAACGACCGAATGGCTTTAGTAGACATGTCAGCAGCCTTTCCCACTGCATGCCACG
AAATGGACACAGATTGAAAGGAAGCCCAATCATTTCCTTTTTTCCCTTGAATGAACGGGGCTATGTCTATTTCATCATTGATATAA
GGAGAGAACAAACAGACAGTCCGTTTTAAAGTAAAAGAAAACAAACTATGATGGTTACTCTCTCGACACAAGAATCACAAGACTT
TTTGCTCCTACCGCAATCCATGACCCCGGTTCCCGCTTTGATTTGTGCAAAGAGAGATATAGTGTTTCACTCTTCTCAAAGATTA
TTGTGTTTGGTTCCTTACAAATCTCTCAAATGATAAGAAGGGCAATACTCCTAACCGCCCTGCAAGGGACATCTCCAATGGTCCA
AATGGCCAGGCATTTTTAAGTTAGAACAAAGATCATATATTTATGATGAGCACTTTGAACTTATGATGGGAATCTTGTATAAAAAG
AGAAGTAAAACTATGCTGATTCAATTCAAGTGCAAGCAACTTGGCAAACACAAGTCCATCGGGAAGTAAACACATTCCTGTGATT
TGTTTTTGGCGGCAGGCGTCACAAGTGCGCGGCATGCTTCCGGCAGTTCAACAAGATGGAGCACCTGGTGGAGCACATGCGG
GCGGCGCGGCACTCGTCGCACGAGCCCCGCTGCGACATCTGCCGCAAGCACTGCCGCTCCTTCGAGGCGCTCAGGGACCAC
CTCGGCGTCGGCGGCTCCACGCTGCCCAAGGCCGCCTACTGCGCCGACGCCTTCGCCGCGCGCGGCTGCACCATCTGCCTC
CGCGTCCTCGCCAGCGCCGCCGCGCTCGGAGCCCACAGCGCGGCGTGCCGGCTCTCGCGCATCCCACCGCCGATGATGTCG
AGGGCGCTGCAGCATCACCAGCTTCCAGCAGCAACGCAGCCGCATGGAGGAGGAGGAGAAGGAGGCGCGCTGGCGCTGGG
CTGCAAGATGGTCGGCGCCGGGAGCGACGGGTCCCTGGACGTGTGCGCGCGGGTGTGCGTCATCGACGAGCAGGAGAACAT
CCTGTTCGAGGCCTTCGTGAAGCCGCTCCTCCCGGTGACGCACTACCGGTACGAGACGACGGGGATCCGGCCGGAGCACCT
CCGCGACGGCGCGAGCACGACGGTGAAGAACGCGCAGCGGGGGGTGGAGGAGCTGCTGCTCAACGGCGAGCAGCCGTGGA
AGGTGCGCACGAGCCGGGGCAGGGCGAGGCTGCTGGTCGGGCACGGCCTGGACCACGACCTCGACGCGCTGGGCATGGAC
TACCCGGGCTACCTCAAGCGCGACACGGCCACGTACCCGCCGCTGATGAAGACGAGCAAGCTCAGCAACTCGCTCAGGTTCC
TGACGCACAAGTACCTCGGGTACGACATCCAGACGGGGCACCAGCACCCCTTCGAGGACTGCGTCGCCGCCATGCGCCTCTA
CCGCAGGATGCGCGGGCAGCAGCAGCACCCCAGGGGCGGCGGCGGCGACGCCGACGCACCGGCGGCGGCGACCGCGGAC
CAGGCGTTCCCGGCGTGGAGGCAGCGGGAGCTCGAGCGCATGACGCCTGAGGAGCTCCTTCGGCTCTCCACGCCGGACTAC
CGCTGCTGGTGCCTCGATGCGTAG
SEQIDNO: 247
MDSRRESSETLRNKCAACFRQYNRMEHLVEHMKVSYHSVHEPKCGVCGKHCRSFESLREHLIGLLPKVECARVFSVRGCSICLNIFD
SNATVRYHRAACQYSRAAPMPRGGITGRAVALACKMVGGGSDGSMDLCARVCLIGEDEHIIFQTYVKPTLPVTNYRYEVTGIRPEYL
RDAMPLKVAQRRIQEILCNGEPLWKLRPRSYGRAKILVGHGLDHDLERLGLEYPAFMIRDTAKYPPLMKTSKLSNSLKYLTQAYLGYDI
HTGIQDPYEDCVAAMRLYIRMRSQAHPRDYNSGSGEAQNNYPAWRQREFERMSPEELLALSASDYYCWCLDY
SEQIDNO: 248
ACATGTTGGGAAGAGCAATCTCGTTTGATAACATCCTAGAACTTTTGATAGTGCAAATTGCTGCTGCTTAATAGGCAGTAGTTAG
GCATAAGACAGGTGTATTTCTCTCTCGAACCATCGTCGCCCTATGAATGAAAGACTGAAAGTGACAACCTTAGGAGTACTAGGT
ACTTAGAATGACCAGAAAAGGGAGTTGCCGGTACTTAGCTTACGATAACTGCAACTTTAGAATGGCAAGGCTGATCCCTCGTGA
AGGCTTTATCTCTCGGCATTGCCTATAAATAGCCTCCTGGCGTCGCCCATCCTCTCGCCAGCCTCGAGATCGATCTAGGCCTCT
CAGGGACCTTGATCCCCCAAGTCCCGAATTGTAGGAATCTGAGGGCTGATGGACAGCAGGAGGGAGTCCTCGGAGACCTTGA
GGAACAAATGCGCAGCCTGCTTCAGGCAGTACAACAGGATGGAGCACCTGGTGGAGCACATGAAGGTCTCGTACCACTCGGT
GCACGAGCCCAAGTGCGGCGTCTGCGGGAAGCACTGCCGCTCCTTCGAGTCGCTCAGGGAGCATCTCATCGGGCTGTTGCCG
AAGGTGGAGTGCGCGCGGGTCTTCAGCGTCCGCGGCTGCAGCATCTGCCTCAACATTTTTGACAGCAACGCCACCGTCAGATA
CCACCGTGCGGCCTGCCAATACTCTCGTGCTGCTCCGATGCCCAGGGGTGGCATAACCGGTCGTGCGGTTGCCCTGGCTTGC
AAAATGGTAGGGGGAGGGAGTGACGGTTCAATGGACCTTTGTGCAAGGGTGTGCCTCATTGGAGAAGATGAGCACATCATCTT
CCAGACCTATGTCAAACCTACACTTCCTGTCACCAACTACAGGTATGAAGTAACTGGGATAAGGCCAGAGTACCTGAGGGATGC
AATGCCGCTTAAGGTTGCGCAGAGGAGAATCCAGGAAATCCTGTGCAACGGGGAGCCACTGTGGAAGTTACGCCCAAGGAGC
TATGGGAGGGCAAAGATCCTTGTTGGTCATGGCCTGGACCATGACCTTGAGCGCCTAGGGTTGGAGTACCCGGCATTCATGAT
TAGGGATACTGCGAAATACCCACCACTAATGAAGACTAGCAAGCTGAGTAACTCCCTGAAGTACCTTACACAAGCATACCTTGG
GTATGATATCCATACTGGCATTCAGGACCCCTACGAGGACTGTGTTGCAGCAATGAGGCTGTACATCAGGATGAGATCACAAGC
TCACCCAAGAGATTACAACTCCGGCTCAGGCGAGGCCCAGAACAACTATCCAGCCTGGAGGCAGAGAGAGTTCGAGAGGATG
AGCCCAGAAGAACTCCTGGCACTTTCAGCATCAGACTACTATTGCTGGTGCCTGGATTACTAAACTGATAGTCTATAAAGAAAAT
AAGGCAGGCCAACATTGTTGGTTCATTCATATTCCGTCTATGATAATAAATCTTTAGATGTACAAGTAGCTTGCGGCTTGACTATA
TATTGGGGTTAATTATGATGTATAAACAAATGATTTTATATATATGTAATGATGCTTTCATATAAAGATTGCTGTTGTAGATCTGGA
TAACAAGTATTATTATGATTTATGATGCATATGGAAAGAGGGTTTCCCGCTTGCTTTTTATAAATTCTAAAATGAGAAAAGCAGCA
GCACATCTTGATTTATGTACATAAAACGAATGGATGATTGTACATTTTGTGCAGCTTGTCAGACTATCTCAGTGCTATACAAGTAT
ATCTGCATTAGCCTCAGTGCATCACACGTATGTAAACCGGGCGATTCAAGTTTCTGAACTGTTGAAGAAAAGTCTTACACTGGA
GTCGTTGGATTTCCGGTACTGCTTAGTTTCTGTGGGTGGATTCTTAGGTTTAGGCTCGGGTGTTTTTTCCTGCTTGCCATTCTTT
TTCTTGTCCTTGGACTGGCTCCTATTTAGCTTGATGAACTCAAGACACTGTCGAATGATTGTCAGCACCGTGAAATGAGTCTCAA
AGTCGTTCTTGATGACAGGGGGGTGGTGGAGGTTGGGCCCATTGTTGCTCCAGGTGTAGTTCTCAATGAAGTAGACACAATCA
GTTCTAGCCCTGAACGAAACCTCCCGTAGAAGCTCTTCGGCCTGCCTAATGGATCTCATGGCCTTCTTAAAAGTCAATACGATG
ATCAAATTAAG
SEQIDNO: 249
ACATGTTGGGAAGAGCAATCTCGTTTGATAACATCCTAGAACTTTTGATAGTGCAAATTGCTGCTGCTTAATAGGCAGTAGTTAG
GCATAAGACAGGTGTATTTCTCTCTCGAACCATCGTCGCCCTATGAATGAAAGACTGAAAGTGACAACCTTAGGAGTACTAGGT
ACTTAGAATGACCAGAAAAGGGAGTTGCCGGTACTTAGCTTACGATAACTGCAACTTTAGAATGGCAAGGCTGATCCCTCGTGA
AGGCTTTATCTCTCGGCATTGCCTATAAATAGCCTCCTGGCGTCGCCCATCCTCTCGCCAGCCTCGAGATCGATCTAGGCCTCT
CAGGGACCTTGATCCCCCAAGTCCCGAATTGTAGGAATCTGAGGGCTGATGGACAGCAGGAGGGAGTCCTCGGAGACCTTGA
GGTAAGATGATAAGAGATGCTATTCTTTCAGCTTTCATTGATATGCGTACTACTGCAAATGGCCGTGCACTCAGACTGATTAAGT
AGTGCATGCATGGCATACTGCACCTGTCCCTTGTGGGCATATATATGCAATATATTTAAACAGTGCCCAAGATATCATCAAGTCA
GTTCACTAATCTATGCAAGGCTTGAAACATGCATGTCCATTTCTTGTAACCATATGATCCTGTCAGACTGCCGTTGCGAGAGCTG
CTAGCTGTTCATGCATGAAACTGCTCGTGCAAGGATCAGCTTTGCCTCCTGCATTCGATCCTGTATATGTTCCTGAAGGCTAGAA
TCCATACATCATGATCACAAACATATATATGAATGCCCGCCTCATGCAAAGTCGTGTCCCCCATGATCATCAGGAACAAATGCGC
AGCCTGCTTCAGGCAGTACAACAGGATGGAGCACCTGGTGGAGCACATGAAGGTCTCGTACCACTCGGTGCACGAGCCCAAG
TGCGGCGTCTGCGGGAAGCACTGCCGCTCCTTCGAGTCGCTCAGGGAGCATCTCATCGGTATGCAGAAATTAAGCTTTTGCCG
AGAAGAAAAGACGGCAGCTTACATGCAGCCCAACAAGCACGATTTCAATTCTGACGACGCTTCTTCTTCTTCTTCTCCATCTTCC
TTCAGGGCTGTTGCCGAAGGTGGAGTGCGCGCGGGTCTTCAGCGTCCGCGGCTGCAGCATCTGCCTCAACATTTTTGACAGCA
ACGCCACCGTCAGATACCACCGTGCGGCCTGCCAATACTCTCGTGCTGCTCCGGTAATGATATCGATCGATCTGTTGGTCTCTA
CCCTATTCATTATTCATATGCCGTATACTCAGTTCCACTTCAGAGCTCATCAGATGCATAGGCTTCGCTTTGTCGGCTTTGGGCT
TTGGTTGATACTATAGATTCTAATTTTTGTTCCACCGTCCTATGTGGTCCCAGCAGATGCCCAGGGGTGGCATAACCGGTCGTG
CGGTTGCCCTGGCTTGCAAAATGGTAGGGGGAGGGAGTGACGGTTCAATGGACCTTTGTGCAAGGGTGTGCCTCATTGGAGA
AGATGAGCACATCATCTTCCAGACCTATGTCAAACCTACACTTCCTGTCACCAACTACAGGTAATCCTACTTCTCATGCAGTTGC
CATGCATCATGTTACTAGTCTACTAGCAACTCTAGTCGATGGTTGAGGAGAAATTGTGAGACCAGCAAAACCAGAATGAAAGAG
TCGGCAATCTTTCAGGGAGTACCACTATATATGTGCTTGAAAGTTGAAGTTGGACAGCGAAGATATTTGTATGGACTACTTTCGT
AACGACACGAACTGATGGCTATATGCAACATGTTTTTCAGGTATGAAGTAACTGGGATAAGGCCAGAGTACCTGAGGGATGCAA
TGCCGCTTAAGGTTGCGCAGAGGAGAATCCAGGAAATCCTGTGCAACGGGGAGCCACTGTGGAAGTTACGCCCAAGGAGCTA
TGGGAGGGCAAAGATCCTTGTTGGTCATGGCCTGGACCATGACCTTGAGCGCCTAGGGTTGGAGTACCCGGCATTCATGATTA
GGTGAGGAGCAGGGAGGAAAAAAATCACTGTATTTTTCTTTTCTTCCTAACATCTATAGGTCTTGCTTGTTGAGCTATGTATGGT
TCAGGAATCAGTTACCACAGTTATTTTGCCCTTTTTGACAGAAATTGTTATGCATATGCAGGGATACTGCGAAATACCCACCACT
AATGAAGACTAGCAAGCTGAGTAACTCCCTGAAGTACCTTACACAAGCATACCTTGGGTATGTCACTTCTTGGATGATCTATACA
ATCAGAATCAGCCTACTATTATTTAATATCTGGGTAATAATGATTCTAAGGAGATTGTATCCTCTTGTAGGTATGATATCCATACT
GGCATTCAGGACCCCTACGAGGACTGTGTTGCAGCAATGAGGCTGTACATCAGGATGAGATCACAAGCTCACCCAAGAGATTA
CAACTCCGGCTCAGGCGAGGCCCAGAACAACTATCCAGCCTGGAGGCAGAGAGAGTTCGAGAGGATGAGCCCAGAAGAACTC
CTGGCACTTTCAGCATCAGACTACTATTGCTGGTGCCTGGATTACTAAACTGATAGTCTATAAAGAAAATAAGGCAGGCCAACAT
TGTTGGTTCATTCATATTCCGTCTATGATAATAAATCTTTAGATGTACAAGTAGCTTGCGGCTTGACTATATATTGGGGTTAATTAT
GATGTATAAACAAATGATTTTATATATATGTAATGATGCTTTCATATAAAGATTGCTGTTGTAGATCTGGATAACAAGTATTATTAT
GATTTATGATGCATATGGAAAGAGGGTTTCCCGCTTGCTTTTTATAAATTCTAAAATGAGAAAAGCAGCAGCACATCTTGATTTAT
GTACATAAAACGAATGGATGATTGTACATTTTGTGCAGCTTGTCAGACTATCTCAGTGCTATACAAGTATATCTGCATTAGCCTCA
GTGCATCACACGTATGTAAACCGGGCGATTCAAGTTTCTGAACTGTTGAAGAAAAGTCTTACACTGGAGTCGTTGGATTTCCGG
TACTGCTTAGTTTCTGTGGGTGGATTCTTAGGTTTAGGCTCGGGTGTTTTTTCCTGCTTGCCATTCTTTTTCTTGTCCTTGGACTG
GCTCCTATTTAGCTTGATGAACTCAAGACACTGTCGAATGATTGTCAGCACCGTGAAATGAGTCTCAAAGTCGTTCTTGATGACA
GGGGGGTGGTGGAGGTTGGGCCCATTGTTGCTCCAGGTGTAGTTCTCAATGAAGTAGACACAATCAGTTCTAGCCCTGAACGA
AACCTCCCGTAGAAGCTCTTCGGCCTGCCTAATGGATCTCATGGCCTTCTTAAAAGTCAATACGATGATCAAATTAAG
SEQIDNO: 250
MDTRKESSETLRNKCAACYRQYNRMEHLVEHMKVNYHSVHEPRCGVCGKHCRSFESLREHLIGPLPKVECARVFSVRGCSICLNIF
DSNAAVRYHRAACQYTRAAPMPMGSITGRAVSLACKMVGGGSDGSVDLCARVCLTGEDENVIFQTYVKPTAPVTNYRYEVTGIRPE
YLRDAMPLKIAQRRIQEILCNGEPLWKLRPRSYGRAKILVGHGLDHDLERLGLEYPAFMIRDTAKYPPLMKTSKLSNSLKYLTQAYLGY
DIQTGIQDPYEDCVAAMRLYIRMRSQAHPRDYNSGSGEAQNNYPAWRQRELERMSPEELLALSASDYYCWCLDY
SEQIDNO: 251
CGAGCTCGATCTCTTCTCAGGAACCCTTGACCAAAGTGTAGGAGTCTGAGTCATGGACACCAGGAAGGAGTCCTCGGAGACCT
TGAGGAACAAATGCGCAGCCTGCTACAGGCAGTACAACAGGATGGAGCACCTTGTGGAGCACATGAAGGTGAACTACCACTCG
GTGCACGAGCCTAGGTGTGGCGTCTGCGGCAAGCACTGCCGCTCCTTCGAGTCGCTCAGGGAGCATCTCATCGGGCCTTTGC
CAAAGGTGGAGTGCGCGCGGGTCTTCAGTGTTCGCGGCTGCAGCATCTGCCTCAACATCTTTGACAGCAACGCCGCTGTCAGA
TATCACCGTGCTGCCTGCCAATACACCCGTGCTGCTCCGATGCCCATGGGCAGCATAACCGGCCGTGCGGTTTCTCTGGCCTG
CAAAATGGTTGGAGGAGGGAGTGACGGCTCAGTGGACCTTTGTGCAAGGGTGTGCCTCACTGGAGAAGACGAGAATGTCATCT
TCCAGACCTATGTCAAACCTACAGCTCCTGTTACCAACTACAGGTATGAAGTAACTGGGATAAGGCCAGAGTACTTGAGGGACG
CAATGCCACTGAAGATTGCACAGAGGAGGATTCAGGAGATCCTGTGCAATGGGGAGCCTCTGTGGAAGTTACGCCCAAGGAGT
TATGGAAGGGCAAAGATTCTGGTTGGCCATGGCCTGGACCATGATCTGGAGCGCCTAGGGTTGGAGTACCCAGCATTCATGAT
CAGGGACACTGCAAAATACCCACCTCTGATGAAGACTAGCAAGCTGAGTAACTCCCTGAAGTACCTTACACAAGCATATCTGGG
ATATGACATTCAAACAGGCATTCAGGACCCCTACGAGGATTGCGTGGCAGCAATGAGGCTGTACATCAGGATGCGATCACAAG
CTCACCCGAGAGATTACAACTCTGGTTCAGGTGAGGCCCAGAACAACTACCCAGCCTGGAGGCAGAGAGAGCTGGAGAGGAT
GAGCCCAGAAGAACTCCTTGCACTTTCAGCATCAGACTACTATTGTTGGTGCCTGGATTACTAATTGATCAGATACAATGTGAAT
AAGGCAGGCCAATATTGTTGGTCCATTCATATTCTGTCTATGGTAATAAAACCTTTAGATGTACAAGTAGCTTGCGGCTTGACTA
TACTGGGGTCAATTATAACGTATACACAAGGGATTCTATATACATGTAATGCTTTCTTATAAATATTGGTGATTTAGAGATGTAAG
AAGTGATATTATGTTAATGATATTATATGGTAAGAGGGGTTTTCCCCCATGCTTTATGAATCCAAAAAAAATGAGAAAACCAAGAG
TA
SEQIDNO: 252
CGAGCTCGATCTCTTCTCAGGAACCCTTGACCAAAGTGTAGGAGTCTGAGTCATGGACACCAGGAAGGAGTCCTCGGAGACCT
TGAGGTAAGATGATAAGATATGCTATTCTTCCATTTTTTTTCCAGCATTGATATCAAGCTATACTGCACCAATCCCTTATAGGCAT
ATGCAGAAAACTCATGCTAAGGCACCTCAATACCAAACTAATTAAGGCTGGAAACATGTCCATTTATTGTAACCATGAAACCAGC
CATATTGCAGTTGATCCTGCATTTGGTCCTACGTCATCCTGTAATCAGGAAGGCTAGGTTAATCTACATCAAACTGAAGCATATG
CATGTATGTCCCCCATGATCAGGAACAAATGCGCAGCCTGCTACAGGCAGTACAACAGGATGGAGCACCTTGTGGAGCACATG
AAGGTGAACTACCACTCGGTGCACGAGCCTAGGTGTGGCGTCTGCGGCAAGCACTGCCGCTCCTTCGAGTCGCTCAGGGAGC
ATCTCATCGGTAAGCCCAAAAGAACTTGCTGAGAGGAAAAGATGGCAGCTTACTTCTCAACGAGCGTGGTTTCTGATGCTTCTT
GTTCTCCATCTTCAGGGCCTTTGCCAAAGGTGGAGTGCGCGCGGGTCTTCAGTGTTCGCGGCTGCAGCATCTGCCTCAACATC
TTTGACAGCAACGCCGCTGTCAGATATCACCGTGCTGCCTGCCAATACACCCGTGCTGCTCCGGTAATGGCTTACTGATTTCCA
CTCTATTCATTCATATTATCATACATACCGTATATTCAGTCCATTTCAGAACTCAGATGCATAGACTTCCCTTTGTTGCGAATTTGG
TTGATACTAGATTCTAACATGTTTTTCCCTTTCTATGCGGTCCCAGATGCCCATGGGCAGCATAACCGGCCGTGCGGTTTCTCTG
GCCTGCAAAATGGTTGGAGGAGGGAGTGACGGCTCAGTGGACCTTTGTGCAAGGGTGTGCCTCACTGGAGAAGACGAGAATG
TCATCTTCCAGACCTATGTCAAACCTACAGCTCCTGTTACCAACTACAGGTAACATTTCATTCCATCAGTTCTCCAGACCAATTGT
CTACCATGCCTAGATGATGGTAGAAGAGAAAGTACCAGTACAGCTATGCCTGAAACTAGACAATAGAGATTTGGATGGACAGCT
TTCTGAAATGAGATGACTGATTACTTGTGTGTTCAGGTATGAAGTAACTGGGATAAGGCCAGAGTACTTGAGGGACGCAATGCC
ACTGAAGATTGCACAGAGGAGGATTCAGGAGATCCTGTGCAATGGGGAGCCTCTGTGGAAGTTACGCCCAAGGAGTTATGGAA
GGGCAAAGATTCTGGTTGGCCATGGCCTGGACCATGATCTGGAGCGCCTAGGGTTGGAGTACCCAGCATTCATGATCAGGTGA
GGAGCACAAGATAGCTAAGTCAGTGGATTATGGTATTTCTTCTTTCTAACATATACATATATTGTTTACTGAGCTATGGTTCAGGA
ATCAGGAGTCAAAGTTGCCACGTCAGTTTTGTCTTTTGACACACTATTGTGCATGTGCAGGGACACTGCAAAATACCCACCTCT
GATGAAGACTAGCAAGCTGAGTAACTCCCTGAAGTACCTTACACAAGCATATCTGGGGTATGTCACTAGCTAGGCTAATCTACA
CAATCAACCTACTCTTAATATCTTCGTAATGCTAAATAGATTATTTTTGCATCCTATTGTAGATATGACATTCAAACAGGCATTCAG
GACCCCTACGAGGATTGCGTGGCAGCAATGAGGCTGTACATCAGGATGCGATCACAAGCTCACCCGAGAGATTACAACTCTGG
TTCAGGTGAGGCCCAGAACAACTACCCAGCCTGGAGGCAGAGAGAGCTGGAGAGGATGAGCCCAGAAGAACTCCTTGCACTT
TCAGCATCAGACTACTATTGTTGGTGCCTGGATTACTAATTGATCAGATACAATGTGAATAAGGCAGGCCAATATTGTTGGTCCA
TTCATATTCTGTCTATGGTAATAAAACCTTTAGATGTACAAGTAGCTTGCGGCTTGACTATACTGGGGTCAATTATAACGTATACA
CAAGGGATTCTATATACATGTAATGCTTTCTTATAAATATTGGTGATTTAGAGATGTAAGAAGTGATATTATGTTAATGATATTATA
TGGTAAGAGGGGTTTTCCCCCATGCTTTATGAATCCAAAAAAAATGAGAAAACCAAGAGTA
SEQIDNO: 253
MDSRRESSETLRNKCAACYRQYNRMEHLVEHMKVNYHSVHEPRCGVCGKHCRSFESLREHLIGPLPKVECARVFSVRGCSICLNIF
DSNAAVRYHRAACQYTRAAPMPRGSITGRAVALACKMVGGGSDGSVDLCAMVCLIGEDENIIFQTYVKPTAPVTNYRYEVTGIRPEY
LRDAMPLKVAQRRIQEILCNGKPLWKLRPRNYGRAKILVGHGLDHDLERLGLEYPAFMIRDTAKYPPLMKTSKLSNSLKYLTQAYLGY
DIQTGIQDPYEDCVAAMRLYIRMRSQAHPRDYNSGSGEVQNNYPAWRQRELERMSPEELLALSASDYYCWCLDY
SEQIDNO: 254
GCTCGATCTCTACTCAGGAACCCTTGACCCAAGTGTAGGAATCTGAGTCATGGACAGCAGGAGGGAGTCCTCGGAGACCTTGA
GGAACAAATGCGCGGCCTGCTACAGGCAGTACAACAGGATGGAGCACCTGGTGGAGCACATGAAGGTGAACTACCACTCGGT
GCACGAGCCCAGGTGCGGCGTCTGCGGCAAGCACTGCCGCTCCTTCGAGTCACTCAGGGAGCATCTCATCGGGCCTTTGCCG
AAGGTGGAGTGCGCGCGGGTCTTCAGCGTTCGCGGCTGCAGCATCTGCCTCAACATCTTTGACAGCAACGCCGCCGTCAGAT
ATCACCGTGCTGCCTGCCAATACACCCGTGCTGCTCCGATGCCCAGGGGCAGCATAACTGGCCGTGCGGTTGCTCTGGCTTGT
AAAATGGTTGGAGGAGGGAGTGACGGCTCAGTGGACCTTTGTGCAATGGTGTGCCTCATTGGAGAAGACGAGAATATCATCTT
CCAGACCTATGTCAAGCCTACAGCTCCTGTTACCAACTACAGGTATGAAGTAACTGGGATAAGGCCAGAGTACTTGAGGGATGC
AATGCCACTGAAGGTTGCACAAAGGAGGATCCAAGAGATCCTGTGCAATGGGAAGCCGCTGTGGAAGTTACGCCCAAGGAATT
ATGGGAGGGCAAAGATTCTGGTTGGCCACGGCCTGGACCATGATCTTGAGCGCCTAGGGTTGGAGTACCCAGCATTCATGATC
AGGGACACTGCAAAATACCCGCCTCTGATGAAGACTAGCAAGCTGAGTAACTCCCTGAAATACCTTACACAAGCATATCTGGGG
TATGACATTCAAACAGGCATTCAGGACCCCTACGAGGATTGTGTGGCAGCAATGAGGCTGTACATCAGGATGCGATCACAAGC
TCACCCGAGAGATTATAACTCTGGTTCAGGTGAGGTCCAGAACAACTACCCAGCCTGGAGGCAGAGAGAGCTGGAGAGGATGA
GCCCAGAAGAACTCCTTGCACTTTCAGCATCAGACTACTATTGCTGGTGCCTGGATTACTAAATTGATTGGCTATAAAGCGAATA
AGGCAGGCCAACATTGTTGGTCCATTCATATTCCGTCTATGACAATAATCTTTAGATGTACAAGTTGCTTGCGGCTTGACTATAC
TGGGGTTAATTATAACATATAAACAAGGGATTCTATATACATGTAATGCTTTCTTATAAATATTGGTGATTTAGAGATGTAAGAAGT
GATATTATTATCTTAATGATATTATATGG
SEQIDNO: 255
GCTCGATCTCTACTCAGGAACCCTTGACCCAAGTGTAGGAATCTGAGTCATGGACAGCAGGAGGGAGTCCTCGGAGACCTTGA
GGTAAGATGATAAGATATGCTATTCTTTCAGTTTTCAGCATTGATATCAAGCTATACCACACCTCTCAAGCTAAGGCACCTCAATT
ACCAATCCTTGTCCATTTCTTGCAACCATGAAACAAGTCAGATTACAGTCGATCCTGCATTATATTTGATCCTACGTCGTCCTGTA
ACCAGGAAGGCTATAGGTTAATCTATGCATCAAAATGAAGCATATGCATGTATGTCCTCCATGGTCAGGAACAAATGCGCGGCC
TGCTACAGGCAGTACAACAGGATGGAGCACCTGGTGGAGCACATGAAGGTGAACTACCACTCGGTGCACGAGCCCAGGTGCG
GCGTCTGCGGCAAGCACTGCCGCTCCTTCGAGTCACTCAGGGAGCATCTCATCGGTATGCAAAAATGAATTTGCCAAGAGGAA
AATATAGCAGCTTACTGCTGAACGAGTGTGGTTTCTGACTCTTCTTGTTCTCGATCTTCAGGGCCTTTGCCGAAGGTGGAGTGC
GCGCGGGTCTTCAGCGTTCGCGGCTGCAGCATCTGCCTCAACATCTTTGACAGCAACGCCGCCGTCAGATATCACCGTGCTGC
CTGCCAATACACCCGTGCTGCTCCGGTAATGGTTTATTGATTTCTACTCTATTCATTCATGTATACAGTATATTCAGTCCATTTCA
GAACTCGGATGCATAGACTTCCCTTTGTTCTGACTTTGGTTGATACTAGATTCTAACATTTGTTTTCTTTTCCATGCGGTCCCAGA
TGCCCAGGGGCAGCATAACTGGCCGTGCGGTTGCTCTGGCTTGTAAAATGGTTGGAGGAGGGAGTGACGGCTCAGTGGACCT
TTGTGCAATGGTGTGCCTCATTGGAGAAGACGAGAATATCATCTTCCAGACCTATGTCAAGCCTACAGCTCCTGTTACCAACTA
CAGGTAACATTTCATTCCATCACTTCTCCAGACCAATTGTCTACCATGTCTAGATGACGGTAGAAGATAAATTAGCAGGCCAGCG
AAACCAGAAAGAAAGCATTACCGATCATTCAATGAACACTATGCTTGAAACTAGATAGTAGAGATTTGGATGGACTGCTTTCTGA
AAATGAGATGACCAATTACTTGTGTGTTCAGGTATGAAGTAACTGGGATAAGGCCAGAGTACTTGAGGGATGCAATGCCACTGA
AGGTTGCACAAAGGAGGATCCAAGAGATCCTGTGCAATGGGAAGCCGCTGTGGAAGTTACGCCCAAGGAATTATGGGAGGGC
AAAGATTCTGGTTGGCCACGGCCTGGACCATGATCTTGAGCGCCTAGGGTTGGAGTACCCAGCATTCATGATCAGGTGAGGAG
CACAAGATAGCTAAGTCATTGTTTTATAGTATTTCTTCTTTCTAACATATATAGATCTTGTTTACTGAGCTATGGTTCAGGAATCAG
GAGTCAAAGTTTCCACGTCAGTTGTCTTTTGACACATTATTATACATGTGCAGGGACACTGCAAAATACCCGCCTCTGATGAAGA
CTAGCAAGCTGAGTAACTCCCTGAAATACCTTACACAAGCATATCTGGGGTATGTCACTAGCTAGGCTAATGTACACAATCAACC
TACTCTTTAATATTTGTGTAATGCTAAATAGATTGTTTTTGCATCCTATTGTAGGTATGACATTCAAACAGGCATTCAGGACCCCT
ACGAGGATTGTGTGGCAGCAATGAGGCTGTACATCAGGATGCGATCACAAGCTCACCCGAGAGATTATAACTCTGGTTCAGGT
GAGGTCCAGAACAACTACCCAGCCTGGAGGCAGAGAGAGCTGGAGAGGATGAGCCCAGAAGAACTCCTTGCACTTTCAGCAT
CAGACTACTATTGCTGGTGCCTGGATTACTAAATTGATTGGCTATAAAGCGAATAAGGCAGGCCAACATTGTTGGTCCATTCATA
TTCCGTCTATGACAATAATCTTTAGATGTACAAGTTGCTTGCGGCTTGACTATACTGGGGTTAATTATAACATATAAACAAGGGAT
TCTATATACATGTAATGCTTTCTTATAAATATTGGTGATTTAGAGATGTAAGAAGTGATATTATTATCTTAATGATATTATATGG
SEQIDNO: 256
MDYRNESSDTLRNKCAACYRQFNKKEHLVEHMRTSYHSVHEPMCGVCKKHCRSFESLREHLIGPLPKAECERIFKERGCDICLSILG
NRTSLRAHRESCLSRPNNNGLLYRMANLGIQDELRIDNSRGRVVALACKMVGGGSDGSLDLCARVCLIDEHERILFHSYIKPNIPVTN
YRYEMTGIRPEYLRDAMPLKNVSRKIQEFLCNGEPIWQIRSKGGRSRILVGHGLDHDLKCLEMDYPAIKMRDTAKYPPLMKTSKLSNS
LKYLTRAYLGYDIQSGVQDPYEDCVATMKLYMRMKLSQFHKKENYPLATDPQNKNNFASWRQNELERMTPDQLLDISRSDYYCWCL
DSQDY
SEQIDNO: 257
ATGGATTATAGAAATGAGTCTTCCGATACTCTCAGGAACAAGTGTGCAGCGTGCTATAGGCAGTTCAACAAAAAGGAACATCTT
GTGGAACACATGAGGACATCTTATCATTCAGTTCATGAGCCCATGTGTGGTGTTTGCAAAAAACACTGCCGCTCTTTTGAATCTT
TAAGGGAGCATCTAATTGGGCCACTTCCAAAGGCAGAATGTGAAAGAATTTTCAAGGAACGAGGATGTGATATTTGTTTGTCAAT
CCTTGGCAATCGAACTTCTCTACGGGCTCATAGAGAATCATGCCTCTCACGTCCTAATAATAACGGTTTATTGTATCGCATGGCT
AATTTGGGGATTCAAGATGAACTAAGAATTGATAACAGCAGAGGAAGAGTGGTTGCTCTTGCTTGTAAAATGGTTGGTGGTGGA
AGTGATGGATCTTTAGATCTTTGTGCTAGGGTTTGTCTCATTGATGAACATGAAAGAATCCTCTTTCATTCATATATCAAACCAAA
CATTCCTGTCACTAACTACAGGTATGAAATGACGGGTATAAGACCAGAATATTTGAGGGATGCAATGCCTTTAAAAAATGTGTCA
AGAAAAATTCAAGAATTTCTTTGCAATGGGGAACCTATTTGGCAAATCCGTTCTAAAGGTGGAAGGTCTAGGATTCTTGTTGGCC
ATGGTTTGGATCATGATCTTAAATGTTTGGAGATGGATTATCCAGCAATAAAGATGAGGGATACTGCAAAATACCCACCACTTAT
GAAAACAAGCAAGCTCAGTAACTCCCTCAAGTACTTGACCAGAGCTTACCTCGGGTATGATATTCAAAGTGGAGTACAAGATCC
GTATGAAGACTGTGTGGCCACAATGAAACTCTACATGAGAATGAAATTATCACAATTTCACAAAAAAGAGAATTATCCTCTTGCTA
CTGATCCACAAAATAAAAATAACTTTGCATCTTGGAGGCAAAATGAGCTTGAGAGGATGACTCCTGACCAACTATTGGATATTTC
AAGGTCTGATTATTATTGTTGGTGTTTGGACTCACAAGATTATTAA
SEQIDNO: 258
ATGGATTATAGAAATGAGTCTTCCGATACTCTCAGGTAATCTATTCATTTCATTTACGCAAATATTTCATTTTTTTACGTCAACATT
TAGATTCGGGTCTAATGTTTGATCAAACACAGAAATAGTCATCAAACATTAACTTTTGAACCATATATATTATCTAACAAAAAATTG
AAAATTGTAAGAATTTTTTTTAAGAGAATTGCGAGAGAAATTTAAAAAAAAAATGAATTATTTACTAGCTAAACAATTTATTGATGG
GAGTACGTACGTAGTACAGGAACAAGTGTGCAGCGTGCTATAGGCAGTTCAACAAAAAGGAACATCTTGTGGAACACATGAGG
ACATCTTATCATTCAGTTCATGAGCCCATGTGTGGTGTTTGCAAAAAACACTGCCGCTCTTTTGAATCTTTAAGGGAGCATCTAAT
TGGTATGCTAGCTACACTTACCCAAAAAAAGCATCCTTTTTTTTCGAGTTTAAGTTATATATATCGTCACGTTCAATTTTTTTTATTT
GGTCACTAATGTACCGCAGGGCCACTTCCAAAGGCAGAATGTGAAAGAATTTTCAAGGAACGAGGATGTGATATTTGTTTGTCA
ATCCTTGGCAATCGAACTTCTCTACGGGCTCATAGAGAATCATGCCTCTCACGTCCTAATAATAACGTAAGCAATCAATTCATCC
AAAAAAAAAATTAAAATTATTAGAGTAGAATAGGAAAAATCTATGCAAGTACTGATACCATGTTAGATTGTGTGACTGTCTTATTG
CAGGGTTTATTGTATCGCATGGCTAATTTGGGGATTCAAGATGAACTAAGAATTGATAACAGCAGAGGAAGAGTGGTTGCTCTT
GCTTGTAAAATGGTTGGTGGTGGAAGTGATGGATCTTTAGATCTTTGTGCTAGGGTTTGTCTCATTGATGAACATGAAAGAATCC
TCTTTCATTCATATATCAAACCAAACATTCCTGTCACTAACTACAGGTGATCCATTTCAATTAATAATGTAAAAAAAAATCTTAACA
CAATTAATAAATTTCAACTAATTTGTTATAACATTTTTATTTAGTTACTACGTTATTAAACGGAGTAACAATATACTTATTGTTCTAA
AGTTAATTTACTACATAAAATTTAAACTCAAATATTAAAGCACGTTCTCTTATTTTTGGTCTTTTTGCGGCCCATTCCATGACAAGA
ACAACAAGGATGTGTCATGATAGTGTATAATTTTTCTTATATTGACGATGTATATTAACTTAAACTCTGACGAGATAAATATATATG
TTTATAGGTATGAAATGACGGGTATAAGACCAGAATATTTGAGGGATGCAATGCCTTTAAAAAATGTGTCAAGAAAAATTCAAGA
ATTTCTTTGCAATGGGGAACCTATTTGGCAAATCCGTTCTAAAGGTGGAAGGTCTAGGATTCTTGTTGGCCATGGTTTGGATCAT
GATCTTAAATGTTTGGAGATGGATTATCCAGCAATAAAGATGAGGTAAATTAATAATTATTTCATCAATTATATAATTTAAAAATAC
TTTACTGCTATATAGAAAAAACATGAAATTAACTCCTACTCATTACTATTTTATATATTACAATCAAATGTCTTTGGTAATCTAGTTT
ACAAAGTACATACACAGTAGCGTATATTTTATACTAAAATATACATATACTTATTCATTTGATATACATACCGTATACATTAACATAT
ATAGCCAAAGTATATAAATAGTGTATATTTCGACCATATTTACGTGAGTTAATTCCCTTCTAACATTTTATTTTATTTTTTGTAAAGG
GATACTGCAAAATACCCACCACTTATGAAAACAAGCAAGCTCAGTAACTCCCTCAAGTACTTGACCAGAGCTTACCTCGGGTAAT
TTATACATTAAATAGTGCATATATAAAACTTTATCCTTAATAATATTATTAATGTAATTTGTTGTTTTTATAAAAAAATAAAATAAATA
TGAATGGACAATAATAAGGGAAATTGTGTGTAGGTATGATATTCAAAGTGGAGTACAAGATCCGTATGAAGACTGTGTGGCCAC
AATGAAACTCTACATGAGAATGAAATTATCACAATTTCACAAAAAAGAGAATTATCCTCTTGCTACTGATCCACAAAATAAAAATA
ACTTTGCATCTTGGAGGCAAAATGAGCTTGAGAGGATGACTCCTGACCAACTATTGGATATTTCAAGGTCTGATTATTATTGTTG
GTGTTTGGACTCACAAGATTATTAA
SEQIDNO: 259
MEYRNKCAACYRQFNKMEHLVEHMRISYHSVHEPMCLICRKHCRSFESLREHLIGPLPKAECEKIFKERGCDICLTLLASRNALRTHK
ESCQLSRSNNGLLYRMTRLGLGFQDDLKISNSQGKVVALSCKMVGGGNDGSLHLCARVCLIDEHERILFESYVAPNIPITNYRYEISGI
RPEYMRNAMPLKQVSRKIQDYLONGEPIWQIRTRSGRARILVGHNLDHDLKCLEMDYPTIMLRDTAKYPPLMKTSKLSNSLKYLSKAY
LGYEIQTGVQDPYDDCVATMRLYKRMKSQAHIRENYPLAIDPQNRNNFASWKENELVKMMPSQLLEISRSDYYCWCLDS
SEQIDNO: 260
ATGGAGTACAGGAACAAATGTGCAGCATGCTATAGGCAGTTCAACAAAATGGAGCATCTAGTTGAACACATGAGGATATCTTAT
CATTCAGTTCATGAACCTATGTGTTTGATTTGTAGAAAACATTGTCGATCTTTTGAATCTTTGAGAGAACATCTCATTGGGCCATT
GCCAAAGGCTGAGTGTGAGAAGATTTTCAAGGAACGAGGATGTGATATTTGTTTGACACTCCTTGCTAGCCGGAATGCACTAAG
GACTCACAAGGAATCATGTCAACTCTCACGCTCAAATAATGGTTTGCTCTATCGTATGACTAGGTTGGGATTAGGTTTTCAAGAT
GACCTAAAAATTAGCAATAGTCAAGGAAAAGTTGTTGCTCTTTCTTGCAAAATGGTAGGTGGTGGCAACGATGGCTCTCTTCATC
TTTGTGCAAGAGTTTGCCTAATTGATGAACATGAAAGAATCCTCTTTGAATCCTATGTTGCACCAAATATTCCCATCACGAACTAT
AGGTATGAAATATCGGGGATTAGGCCAGAATACATGAGAAATGCAATGCCATTGAAGCAAGTATCGAGAAAAATTCAAGATTATC
TTTGTAATGGTGAACCTATTTGGCAAATTCGTACTAGAAGTGGAAGGGCTAGGATCCTTGTTGGTCACAATTTGGATCATGATTT
AAAATGCTTAGAAATGGACTACCCAACAATAATGTTAAGGGATACAGCAAAATACCCTCCACTAATGAAAACAAGCAAGCTGAGC
AACTCACTCAAGTACTTGAGCAAAGCTTATCTTGGATATGAAATTCAAACAGGAGTTCAAGATCCATATGATGATTGTGTTGCAA
CAATGAGGTTGTACAAGAGAATGAAATCACAAGCTCATATAAGGGAGAATTATCCACTTGCAATTGACCCACAAAATCGTAATAA
TTTTGCATCATGGAAGGAAAATGAACTTGTGAAGATGATGCCTTCACAATTACTAGAAATCTCACGATCTGATTATTATTGTTGGT
GTTTGGATTCCTAA
SEQIDNO: 261
ATGGAGTACAGGAACAAATGTGCAGCATGCTATAGGCAGTTCAACAAAATGGAGCATCTAGTTGAACACATGAGGATATCTTAT
CATTCAGTTCATGAACCTATGTGTTTGATTTGTAGAAAACATTGTCGATCTTTTGAATCTTTGAGAGAACATCTCATTGGTAATCTT
CTTTCTTTTTTCCTTCCTAGCAACAAGTTGCAATAGTATTAGTTTGCAGAAGGGGGTTCGTGGGTTTGGACAAAAAAAATAATATA
TATATATATATATATATGTATATGTATATATATTAACCTGTGAACTCTCTAATAAATTTGATTGACAGCTTAGTAGTAAGAAAGGGG
TGGTGAAAATGATTTTCACGCTAATGGTGTGGCTTCAAACCCCACCATCAAGCATGTTTGCAATATTTTTGTCCTGAAATTTACAA
CCCCAAAATTCTAATCCTGGCTCGGCCTCCGAGTCTAGTTAACTTTTTCTTCTAAATTAGTCATATAATTGTATTTTAGGGCCATT
GCCAAAGGCTGAGTGTGAGAAGATTTTCAAGGAACGAGGATGTGATATTTGTTTGACACTCCTTGCTAGCCGGAATGCACTAAG
GACTCACAAGGAATCATGTCAACTCTCACGCTCAAATAATGTATGTATACTTATTCCAACAACACAACGAACATCAGTTTATTTTA
AAATAAAAAAGATATAATAGATTTTTTTTTTCAATTTCTGGTTATTTAGCATTACCCTTTCTCAAATACTCGTAGTTTTATACATTTG
TTAATTTTCAGGGTTTGCTCTATCGTATGACTAGGTTGGGATTAGGTTTTCAAGATGACCTAAAAATTAGCAATAGTCAAGGAAAA
GTTGTTGCTCTTTCTTGCAAAATGGTAGGTGGTGGCAACGATGGCTCTCTTCATCTTTGTGCAAGAGTTTGCCTAATTGATGAAC
ATGAAAGAATCCTCTTTGAATCCTATGTTGCACCAAATATTCCCATCACGAACTATAGGTAATTGTCGTACACCAGTAATTAGTGT
ATTAAAGTTTGATTAATTAACATAATATAGACGGGTTGATCAAATGTTTAGGTATGAAATATCGGGGATTAGGCCAGAATACATGA
GAAATGCAATGCCATTGAAGCAAGTATCGAGAAAAATTCAAGATTATCTTTGTAATGGTGAACCTATTTGGCAAATTCGTACTAG
AAGTGGAAGGGCTAGGATCCTTGTTGGTCACAATTTGGATCATGATTTAAAATGCTTAGAAATGGACTACCCAACAATAATGTTA
AGGTAAGTCCTTTAATTGTCGAGTTTACACAACATACATGTATAAATAATAATTTGATAAATAAGACAACTAATTAAAATATATTTTA
CGTAGGGATACAGCAAAATACCCTCCACTAATGAAAACAAGCAAGCTGAGCAACTCACTCAAGTACTTGAGCAAAGCTTATCTT
GGGTACGTACGTACGTACGTAAATACTTTAAATTGTTAATTATTGTGACCTTTTATATACTCCAGTTTCTAAATATAAAAAATTATA
TCTAAATTCACACGAGACATTTGTTAGTTTAACCTTGGTACTAATAAGAATCAATCTTAATTGTCTTGTAATTGAATAGATATGAAA
TTCAAACAGGAGTTCAAGATCCATATGATGATTGTGTTGCAACAATGAGGTTGTACAAGAGAATGAAATCACAAGCTCATATAAG
GGAGAATTATCCACTTGCAATTGACCCACAAAATCGTAATAATTTTGCATCATGGAAGGAAAATGAACTTGTGAAGATGATGCCT
TCACAATTACTAGAAATCTCACGATCTGATTATTATTGTTGGTGTTTGGATTCCTAA
SEQIDNO: 262
HKCAACFRQFNKIEHLVDHMRTSFHSHHEPTCAICKKHCRSLDSLREHLIGPLPKQECKNIFATRGCKFCLAIFDSSYSQRLHQERCQ
FSPVNSGLLARFANLGIRDGSTVIDSGRTRGSGAIALACKYVGGGSDGSMDICAKVCLIDEGENVIFYSYVKPVTAVTNYRYETTGIRP
EHIRDAMPVKQVQKKIQEILYNGGEKARILVGDCVEDDLKRLQIGYPSFMIRDTANYPPLMKSSKLCNSLKYLAQVYLGFDLQNGIQDP
YEECIVAMRLYKRMRSQVHRKENYPMASDPQNKNNFAIWRENELQRMSPEQMLEISRSDYYCWCMDLRI
SEQIDNO: 263
GCACAAGTGTGCAGCTTGTTTTAGGCAATTCAATAAGATAGAGCATTTGGTAGATCATATGAGGACTTCTTTTCACTCACATCAT
GAACCAACTTGTGCCATTTGCAAGAAACATTGTCGATCTCTTGATTCTCTTAGAGAACATCTCATTGGGCCACTGCCAAAACAAG
AATGCAAAAACATCTTTGCTACAAGAGGATGCAAGTTTTGCTTGGCCATCTTTGACAGTTCTTACTCACAAAGACTCCACCAAGA
AAGATGCCAATTCTCTCCTGTAAATTCTGGACTTCTTGCTCGTTTCGCCAACTTGGGAATTCGTGACGGTTCAACCGTCATCGAC
AGTGGAAGAACACGAGGGTCAGGGGCCATCGCGCTAGCTTGCAAATATGTAGGTGGAGGAAGTGATGGGTCGATGGACATAT
GTGCTAAGGTTTGCCTTATTGATGAGGGTGAGAACGTCATCTTCTATAGCTATGTCAAGCCCGTAACTGCTGTTACAAATTATAG
GTATGAAACAACTGGAATTCGTCCAGAACACATTCGAGATGCAATGCCAGTGAAACAAGTTCAAAAGAAAATTCAAGAAATCCTC
TACAATGGAGGTGAGAAGGCTAGGATTCTTGTAGGTGATTGCGTCGAAGACGATCTCAAACGCTTACAAATCGGTTACCCATCC
TTCATGATAAGGGACACAGCAAATTATCCACCATTGATGAAGTCAAGCAAGCTTTGCAATTCACTCAAGTATCTAGCTCAAGTGT
ATCTAGGATTTGATCTTCAAAATGGTATACAAGATCCTTATGAAGAATGTATCGTTGCTATGAGATTATACAAGAGAATGAGATCA
CAAGTTCATAGGAAAGAGAACTATCCAATGGCTTCTGACCCACAAAACAAAAATAATTTTGCTATTTGGAGGGAAAATGAGCTTC
AAAGGATGAGTCCAGAACAAATGTTGGAGATTTCTAGGTCTGATTATTATTGTTGGTGCATGGATTTGAGGATTTGA
SEQIDNO: 264
GCACAAGTGTGCAGCTTGTTTTAGGCAATTCAATAAGATAGAGCATTTGGTAGATCATATGAGGACTTCTTTTCACTCACATCAT
GAACCAACTTGTGCCATTTGCAAGAAACATTGTCGATCTCTTGATTCTCTTAGAGAACATCTCATTGGTAATGTCACTCTTCTAAT
TCATAATTGGACTTCATTTTTTAAAAAAATTTGGAACATACCTATGTCAACCAAAATATTTACAAAATTTAATTAAATATCATAGTAT
TTCTACGATGGACCGTGATAGACGCACATAGTGGTCTATCATGTCTATTATCGCAAATAGATTGTGATATATTTTTATTATATTTGT
GAATATTTTTAGAAATTTTGTCATTTAAAATAATTTTTCTAGAAATTAACCCAAAAGTCTAATAAAAGTTACAATATATTTTGTATCT
AGTACTATGTTAAAAATTAATTTCTAAGCAGATGAGTTATGATAAATTTATTGTTAGAATATTATATTTCCGATCGATGAACTTTGA
GTTTCATCAATAAATTTTAAAGATAGTCCCTCTCTATATATATTTAGTTTAGTAAAAAACTAGTCTCTCTTAACTAGCTTTTTCGTTA
TTAATTTTGAGTATATTTTTTTCTCTACTAGTATAAGCTGTCTTCCTATTCAAAATATAAGTACTAAAATAAAATGAAACCTAAAGAA
TATAAATAATCAAATGATATTTTAACGTAGTGTAAAAATTTATAGATCAAGACTCTAACACATGTGAAATAAACATCAATATTTGAG
AAAAAAATGATGTTATTAGGAGTTCGAACACCAAACTTCATTATCTAATATATTATTATGTTAAGTATCTAATATATTATTATGTTAA
GAGCTTAAGCTGATGAGTTTACTATTCTTACGTTTTCGTTATTGTATTGTTATTAGGGCCACTGCCAAAACAAGAATGCAAAAACA
TCTTTGCTACAAGAGGATGCAAGTTTTGCTTGGCCATCTTTGACAGTTCTTACTCACAAAGACTCCACCAAGAAAGATGCCAATT
CTCTCCTGTAAATTCTGTATGAATCCTAAAAAACCATTCTATTTAATCAAACAGAATGCATAAATATATATATATATAATTCTTGTTG
ATTTTAAAATCTTGATGTTATTTTGCTTTTTGGGACATCAGGGACTTCTTGCTCGTTTCGCCAACTTGGGAATTCGTGACGGTTCA
ACCGTCATCGACAGTGGAAGAACACGAGGGTCAGGGGCCATCGCGCTAGCTTGCAAATATGTAGGTGGAGGAAGTGATGGGT
CGATGGACATATGTGCTAAGGTTTGCCTTATTGATGAGGGTGAGAACGTCATCTTCTATAGCTATGTCAAGCCCGTAACTGCTGT
TACAAATTATAGGTAAGTAAAATTAAATTCATGTATATAAATTTGAAGTTTAATATTATACTCCTTTTATTTTTATTTTTTGGAATTTG
AGATTCATTTTTCCGTCTAAATTGTCGATTTTTTTTAGAAAAGTTTAGGAACCAAATAAAAACTCATTTGACTATTTTCGAACTTAA
AACATCAACAACTTTATAATTAATTTACTTCTTTTAAAAGGAAATTGAACCAATAAAGTAATTAATTTACTTTATAATTTTTAGAACT
ATGTGATGGAACTTAAGGTCAACATGTATTTATTATTAGAATTTAATCCCTAGTTAGAATTTAGTCATTATAGTTTAGAACGGCATA
GATCAAGTTTAATCTAGTCTACTCCCGATTTATTGCATCTTATTCAAATTTTTTTGTCAGTTGATATATACTTTGAGTGTTGTTGGG
GCTTCATAGATATAAAACAAAAAATTGGTGGAACTTAGAGTGAACTCAAGTTTTGTTTGGTTTAGTTTAACAGTACTCTTGATAAA
ACCCTAGACAATACCTAATTTTAATAAAAATAATTTACCAAACCATATTGACTTAAATTCAAACTTTGGTCAAACCAAATTTGCAAT
TTAACCTAATAATAATAACCCATAAAATTTTATTAAATTTTCCATTTCTTTAGAAATGGAATTTTATCAAGCACATTCGAGATGTTAA
AATTATATATTGTTTTAACCTTTGATATGTTAAAATTAACATTAGGTATGAAACAACTGGAATTCGTCCAGAACACATTCGAGATGC
AATGCCAGTGAAACAAGTTCAAAAGAAAATTCAAGAAATCCTCTACAATGGAGGTGAGAAGGCTAGGATTCTTGTAGGTGATTG
CGTCGAAGACGATCTCAAACGCTTACAAATCGGTTACCCATCCTTCATGATAAGGTAAATTGCTCGTAATAAAATTTAAATTTGTC
GTAACCTATCAACATAATTCATTTTAAAATTTCATATAAAAAATAACAAACATGATCTTCTAAGGATCAACTATATTTTGCATATATA
TATATATATATATATATATATATATATATATATATATATTATTTNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNTACTATTATTATATATATATATATATATATATATA
TATATATATATATATATATATATATATTACTTTTTTTGTTGGTTGAATGCCAGATAAAATAGTAACTTATTTTTAAAAATAGGTTGAAT
TAGGAAATTCAAACTTCTAACATCTAAACAGATGTTAAAGTTTTTTTTTTCATTCCTAATATAAGCTATTAGTGTTGTTGGTGAAAC
CCTTTTAACATCTTATTGCATGTCGAAGTGAGCTCAACAGTGGCAGATCCAAAAATTTTATATAAGGGTATTGAACATTATATAAT
AAATACACTAAAACATATTTAAAAAATATATCGACGTTTAATCGATGCTTCAATAACATTCGCTACATTTGTAGCAACAAAGAAACA
TCTATAATTTCCACGTGATTTTTTGTAATGTAAATATGTAGATTTTTATGGAATGTTGTTGAGAGTAGAAGTTTTGACCAATGACTT
TGTTGCATTTTTTTCTCTAAATATTTGTTAGATAACAAATGAGGGTTAGGAAATAAAGTAGGGTTTATAGAATGATTTTTTTTTCTC
TTTTTAAAAATTATATTCCTTTTACGAAATCATTTGAAATGAAAATTATTGATATTTGAAATAAAAACTATTTCAAAAAAATAATTGTC
ATAAGTAAGAATCAATTTTTTTTTAGGGCACAACCACTACCTCCCAACTAAAATTTAATTAAAATAAAGTATTATTTAATATATATTG
TAATCTGACACTTCAAAATTAATATTAAAATACAATAATTGAGAATGTGAGAGGCACGTGTCCCTTTCGCCCCTTGAGCTCAAAAC
ATTTAAGTTTGGTTTGATTTAGTTTTCTAGATTTTAATAATAAGTAGCGTTAATCTATAAGCTTAACTATTTAAAAAAAAAAGTAAAA
AAGTTATCAAACAACACCCTTAATTTGTTGATTTCATGCACTGACCCACAGGGACACAGCAAATTATCCACCATTGATGAAGTCA
AGCAAGCTTTGCAATTCACTCAAGTATCTAGCTCAAGTGTATCTAGGGTAAGTTAACCTTTTAAGCACTCGTTTGTTTTTTTAAAG
TCTTCCATAAAACTTATATGCTATTCATTTTTTTCATTTAAGGATTGAGATCCATGAAATAATATATAAATATATCATTTGATGGGTC
AAATTGGTGAAAGTAAGAAGTAAGGCAGCTGATGTATTACAGCAGTACTGGAAAATTTTCCATATGTCGTTTTTCTCTATAGAAAA
TGATTAACTAAGAAGTGTGTATTGTTAATTGTAGATTTGATCTTCAAAATGGTATACAAGATCCTTATGAAGAATGTATCGTTGCT
ATGAGATTATACAAGAGAATGAGATCACAAGTTCATAGGAAAGAGAACTATCCAATGGCTTCTGACCCACAAAACAAAAATAATT
TTGCTATTTGGAGGGAAAATGAGCTTCAAAGGATGAGTCCAGAACAAATGTTGGAGATTTCTAGGTCTGATTATTATTGTTGGTG
CATGGATTTGAGGATTTGA
SEQIDNO: 265
MDHPCETLRHKCEGCFKQFNKIEHLVEHMRISFHSIHEPICSFCHKHFRYFESLREHLIGPLPKQECKNIFNARGCKICLAMFESPLQL
NLHQESCQLRPINFSHENRARLGKTESGGTKGNNEAVALSCTMVGAGIDGSLNICVRVCVVDQNESLIFSTYVNPTLPITNYRYEFTG
VRPEHLRDAMPLKQVQKKIQEFLCNGEQMWKIRPGSTGRARILVGHGLQEYLTSLQIDYPPIMIRDTAKYPPLMKTSKLSNSLHCLTQ
TYLGYSMENGKRLEGYEECVATMRLYMRMKSQVHKREEYPLASDPQNKFNFANWKQLELEKMTPQQLLQISRSDYYCWCLDSTL
SEQIDNO: 266
ATGGATCATCCATGTGAAACTTTAAGGCACAAATGTGAAGGTTGCTTCAAGCAATTCAACAAAATAGAGCACTTAGTAGAGCATA
TGAGAATCTCATTTCACTCAATTCATGAACCAATTTGCTCCTTTTGCCACAAACACTTCAGATACTTTGAATCTCTTAGAGAACAT
CTTATTGGGCCATTGCCAAAGCAAGAATGCAAAAACATATTCAATGCAAGAGGATGCAAAATCTGCTTAGCCATGTTTGAGAGTC
CACTCCAGCTCAACCTTCACCAAGAAAGTTGCCAACTTAGGCCTATAAATTTTAGTCATGAAAATAGGGCACGTTTGGGGAAGA
CAGAAAGTGGTGGAACAAAAGGCAATAACGAAGCAGTGGCATTGAGTTGCACCATGGTGGGCGCTGGCATCGACGGCTCCCT
AAATATTTGCGTTAGGGTTTGTGTTGTTGACCAAAACGAGTCCCTCATCTTTTCTACTTATGTCAACCCAACCCTTCCCATCACAA
ACTATAGGTATGAATTCACAGGTGTTCGTCCAGAACACTTGAGAGATGCAATGCCATTGAAGCAAGTCCAGAAGAAAATTCAAG
AGTTTCTATGCAATGGAGAACAAATGTGGAAGATTCGTCCTGGATCTACTGGAAGAGCAAGAATTCTTGTGGGTCATGGCCTTC
AAGAATACCTTACATCTTTGCAAATTGACTACCCTCCCATTATGATCAGGGATACTGCTAAATATCCACCATTGATGAAGACAAG
CAAGCTAAGCAATTCTCTCCACTGTTTGACACAAACATATCTTGGATACAGTATGGAAAATGGGAAGAGATTAGAAGGTTATGAG
GAGTGTGTTGCAACAATGAGATTGTACATGAGAATGAAATCTCAAGTTCACAAAAGAGAGGAATATCCATTGGCTTCTGATCCAC
AAAATAAGTTTAATTTTGCAAATTGGAAGCAACTTGAGCTTGAGAAGATGACACCCCAACAGTTGTTGCAAATTTCAAGATCTGAT
TACTATTGTTGGTGTTTGGATTCCACCCTTTAA
SEQIDNO: 267
ATGGATCATCCATGTGAAACTTTAAGGTGTGGTCTCTCTCTATTTGTGAGATATGTTTTTTGAAATATTAGTAATGAGTAACAAAG
TTTTGTTGACATTATGAACTCATTGGAATTGGGCAGGCACAAATGTGAAGGTTGCTTCAAGCAATTCAACAAAATAGAGCACTTA
GTAGAGCATATGAGAATCTCATTTCACTCAATTCATGAACCAATTTGCTCCTTTTGCCACAAACACTTCAGATACTTTGAATCTCT
TAGAGAACATCTTATTGGTAATACCTCAAACTTTCATGTTTCAAATTCATGAATTTTCAAGTTTTTCTTATTAATTTTATTTTTGTTTT
AAATGAAAAAATAGAAAGGTGTGTGGTAAGCAATTGATCTTTGTTTCTTATTCTCTATAATTTTTTTAAATCAATGTGAAAAAAAAA
TGTATAGATTATATATATATATATATATATATATATATATTGTATATCNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNATATATATATATATATATATATATATATATATTTGATATCCAT
TGATTTATATTATATTTGTTACATCCTTTTTAACAGGGAGAATGTTTCCATTTTGGTTATGGTTTTATGTAATATACAAACTTAATTA
TATTATTTAATATGACAACATTTAATTTATATAGTCTTTTTTGCTAAATTGAGCACATAAGTCGATTAACATATAAATGTGTTTATAA
CTATGTGTTATTGAAAAAAAATATTCTATTTCCTAATGTTTTCGGTTCTGCGTATAAATTTCATATTTTCAGGGCCATTGCCAAAGC
AAGAATGCAAAAACATATTCAATGCAAGAGGATGCAAAATCTGCTTAGCCATGTTTGAGAGTCCACTCCAGCTCAACCTTCACCA
AGAAAGTTGCCAACTTAGGCCTATAAATTTTGTAAGGGTTCGTCGTGTTTTACGCATTTTTTATATCTCCATGTCTTTTACTCGATT
TATGTATTTGAAAGTATATGAACTAAATGTGTTTCCAATTAAAGTTTAATGATCAAATTATAACTTTTATGAAAGTTTAAGAATTGTC
GGTATTTTTTTAACATGAAAAACTAACTTTTTAGAAAAATCTAAAAATAGGTTGCAACAAGTAACAAATTATTATCAAATAATTGATT
GAGATGCATGAAAAATTTATATTAATCAAAGTAAGCATTATATCTGATACTTGTTATATCAATTTTAAATTTAGACGTTCACAATTTT
CTAAACTATATGTATGAAAAGGAGAAATAAAACTTCACATTAATGTTAAAAGAATTTTACCCCTATTAAAAGAAAAAGAGTTTATTA
TACTCTCATGGTGCTAGAAATCTTATTTCGACTGATTGCAGAGTCATGAAAATAGGGCACGTTTGGGGAAGACAGAAAGTGGTG
GAACAAAAGGCAATAACGAAGCAGTGGCATTGAGTTGCACCATGGTGGGCGCTGGCATCGACGGCTCCCTAAATATTTGCGTT
AGGGTTTGTGTTGTTGACCAAAACGAGTCCCTCATCTTTTCTACTTATGTCAACCCAACCCTTCCCATCACAAACTATAGGTATAT
ATTCAATTTCTCCTTCTTCTTTTTTACAGTAATAACTATTGTTGTAATTTCTTCTGCTTTTTTGTTTTTGTTTTTTTTCCATTTTAATAA
AGAAACACAGTAATAAGATTATATTTAGACCCCGTCTGATAAATTTTTAGTTTTTGAAAATCCAAAGTCCTTCAAGAATTGTTTTTG
CATCTTGAAGACTACTATCTATTTATCACGAATAGTTAGAGAGTGTGATATTTTATTATATTTGTAAATAATTTGAAAAGATTTGTG
ATTTAAAACAAAATTAGGTTTTGATATTAATTAGATTGTGTGGGTGTAGGTATGAATTCACAGGTGTTCGTCCAGAACACTTGAGA
GATGCAATGCCATTGAAGCAAGTCCAGAAGAAAATTCAAGAGTTTCTATGCAATGGAGAACAAATGTGGAAGATTCGTCCTGGA
TCTACTGGAAGAGCAAGAATTCTTGTGGGTCATGGCCTTCAAGAATACCTTACATCTTTGCAAATTGACTACCCTCCCATTATGA
TCAGGTAAAATGTTTTATCCTTATTAAATATTTGACTGCATCTCAAGCTACATGCATATCCATTTTTAGGCCGTTTCTTAACAAGGA
AAATATTTTTTAACATATTTTCTCTACATTCTTAACAAGGAAAATAAAAGTCGAATTTCTAATGAATAGTAATTTATTTAAACTGCAG
GGATACTGCTAAATATCCACCATTGATGAAGACAAGCAAGCTAAGCAATTCTCTCCACTGTTTGACACAAACATATCTTGGGTAA
AGTTTTCCACATGCATAACCCCTGCCTCCCCCAATAATATTGTTATTTTTTTCTTCCTTCACTAGCTAGATACCTCAAAAGTAGTA
GTAAAAATATATATATTTGGTAACAATTTTGTTTTAGATATGAAAAATAGATATCAATCCATAAAAAAAATATATGTTAAGATGAAAG
TAGGGTTTACAAGAGTAAGTTTTTAAAATGAAAAAATAAATAGGACAGCTTAGATGATTTATTGTTAAATCAATTTAATTAAAGCTT
AAGCTGGTGGTCAACTGGGATAAGCACTTTAACAGTCCTCTCACTTTTTCTTTTTTTTTTATTTCATGGTCATGATACAGAGCAGA
AAATCAAATTGCTAATCTTCCACGATGGTAGATTTAGATAGAAAATTATATTAAATAAAAATTTAATTTTTAATATATCATGTCAAAT
CATCAATCAACTCGAATAATTAAATTAGTTAATGGATTTTGAGTAATTTAATTATATATACAATACTCTAACATTTATAAACAAATTA
AAGGTAACTAAACAAAATATTTTAGAATGTTAATCAATAAATGTATTAGAATAGATTTGTTGGAATGAATTAAAAAATTTAATAAGTT
TATAAACTTTAAACATTTTACGTCTAATTATATGGTATTGTATATTTTTTGGAATTTTGTTATTCAGATACAGTATGGAAAATGGGAA
GAGATTAGAAGGTTATGAGGAGTGTGTTGCAACAATGAGATTGTACATGAGAATGAAATCTCAAGTTCACAAAAGAGAGGAATAT
CCATTGGCTTCTGATCCACAAAATAAGTTTAATTTTGCAAATTGGAAGCAACTTGAGCTTGAGAAGATGACACCCCAACAGTTGT
TGCAAATTTCAAGATCTGATTACTATTGTTGGTGTTTGGATTCCACCCTTTAA
SEQIDNO: 268
MDSETQRNKCAACYRQFNKKEHLVEHMRISYHSVHEPMCGICGKRCRSFESLRDHLIGPLPKVECERVFRDHGCTICLTVLNSPNSL
RVHQDRCQLSRGNNGVLHRFANMGIHDDLRIDSGKTRAVALACKMVGGGSDGSLDLCARVCIIDEYENILFHSYVKPQLPVTNYRYE
TTGVRPEYLRDAMPLRHVQRKIQDFLCNGEPIWKIRSRGGKARILVGHGLEHDMKCLELEDTAKYPPLMKTSKLSNSLKYLTKSYLGY
DIQNGIQDPYDDCVATMRLYRRMRSQAHRMEDYPLATDSQNSNNFASWRQGELERMSPNELLAISRSDYYCWCLDSKDVA
SEQIDNO: 269
ATGGATTCAGAGACCCAAAGGAACAAATGTGCAGCATGCTATAGGCAGTTTAACAAAAAGGAGCACTTGGTGGAACACATGAG
GATTTCATATCACTCGGTTCATGAACCCATGTGTGGAATTTGTGGGAAACGTTGTCGCTCATTTGAATCTCTCCGTGATCATCTT
ATAGGACCATTGCCAAAGGTTGAATGTGAAAGGGTGTTTAGAGACCATGGATGTACCATTTGCTTAACCGTCCTTAATAGCCCA
AACTCTCTAAGGGTTCACCAAGATAGATGTCAACTATCACGTGGAAATAACGGTGTACTACATAGATTTGCTAACATGGGAATTC
ATGATGACTTGAGAATCGATAGTGGGAAAACAAGGGCAGTTGCACTTGCTTGCAAAATGGTTGGTGGTGGAAGTGATGGTTCTT
TAGATCTTTGTGCAAGAGTTTGTATCATCGATGAATATGAGAACATACTCTTTCATTCATATGTTAAGCCACAACTTCCCGTTACA
AACTATAGGTATGAGACAACAGGCGTTCGACCAGAGTACCTTAGGGATGCAATGCCACTAAGACATGTACAAAGAAAAATTCAA
GATTTTTTGTGTAATGGGGAACCAATATGGAAGATTCGCTCGAGAGGTGGAAAGGCGAGGATTCTTGTAGGACATGGTTTGGAA
CATGACATGAAATGTTTGGAACTTGAGGATACAGCAAAATATCCACCACTAATGAAGACAAGCAAGCTCAGCAACTCACTCAAGT
ACCTAACAAAATCTTACCTAGGGTATGATATTCAAAATGGGATACAAGATCCTTATGATGATTGTGTTGCAACGATGAGACTTTAT
AGAAGAATGAGATCACAAGCTCATAGAATGGAGGATTATCCTCTGGCTACAGATTCCCAAAACAGTAATAACTTTGCTTCATGGA
GGCAGGGTGAGCTTGAGAGGATGAGTCCTAATGAATTGTTGGCAATCTCAAGGTCTGATTACTATTGTTGGTGCTTAGATAGCA
AAGATGTGGCTTAAAATGAAGGGAAGAACAAGGAAAATATTTTAGTGGGTGAAAAAAAATAGCTAAACTTATTTGAAACTAATAAA
ATATAAATGGAATAAACATGGATACACATATATGTTTG
SEQIDNO: 270
ATGGATTCAGAGACCCAAAGGTAGTAACCATATATAAAATACAAAATATAAAATATACTCACATTGTCATAGAAAAACTAACACAC
AAGTTCTTGAAAATACTACATTTTGATCTTATTATTAACATGTTTTTCTTGTTTACTAGGAACAAATGTGCAGCATGCTATAGGCAG
TTTAACAAAAAGGAGCACTTGGTGGAACACATGAGGATTTCATATCACTCGGTTCATGAACCCATGTGTGGAATTTGTGGGAAA
CGTTGTCGCTCATTTGAATCTCTCCGTGATCATCTTATAGGTATTACATTACTATTATTATATATTTTAACTACTATACTCTATACAT
TTATGGATATTTAGTTATTTACTTATATTAATTACTTCTTTAATTCTCAGGACCATTGCCAAAGGTTGAATGTGAAAGGGTGTTTAG
AGACCATGGATGTACCATTTGCTTAACCGTCCTTAATAGCCCAAACTCTCTAAGGGTTCACCAAGATAGATGTCAACTATCACGT
GGAAATAACGTAACAAATCATATCCTTAAACTTACAAAAAATCATTTTTTTGATGTATATATATATGTATAATTATAATTAATCATAT
ATGTTCCAGGGTGTACTACATAGATTTGCTAACATGGGAATTCATGATGACTTGAGAATCGATAGTGGGAAAACAAGGGCAGTT
GCACTTGCTTGCAAAATGGTTGGTGGTGGAAGTGATGGTTCTTTAGATCTTTGTGCAAGAGTTTGTATCATCGATGAATATGAGA
ACATACTCTTTCATTCATATGTTAAGCCACAACTTCCCGTTACAAACTATAGGTGCTATATATTTTGCCATAGTTTTAATTTTTTTAA
GGGTCTATAGACATATAATCATATAAATATATAAAGTTACTCTTTGCCATCTTTTCCTACGTATGTATATATAATCTTATTTAATTAA
AAAAATTCATAGGTATGAGACAACAGGCGTTCGACCAGAGTACCTTAGGGATGCAATGCCACTAAGACATGTACAAAGAAAAAT
TCAAGATTTTTTGTGTAATGGGGAACCAATATGGAAGATTCGCTCGAGAGGTGGAAAGGCGAGGATTCTTGTAGGACATGGTTT
GGAACATGACATGAAATGTTTGGAACTTGAGTATCCTTTAGTAAAGATAAGGCATGTATCTTTAAGGAATTTATTATTACTATCAT
CTACTTTAATAACTTATGAACTAAGAGTAATATGCACTTGTTATTTGATAGGGATACAGCAAAATATCCACCACTAATGAAGACAA
GCAAGCTCAGCAACTCACTCAAGTACCTAACAAAATCTTACCTAGGGTAATAGATATTCATATATTGCCTTTATTTTTAAAAGTTT
GACATGTACATGTGAATTTGTGATATGTTACTTATTGTTAGTGATGGAATTTCTTATTTTATAGGTATGATATTCAAAATGGGATAC
AAGATCCTTATGATGATTGTGTTGCAACGATGAGACTTTATAGAAGAATGAGATCACAAGCTCATAGAATGGAGGATTATCCTCT
GGCTACAGATTCCCAAAACAGTAATAACTTTGCTTCATGGAGGCAGGGTGAGCTTGAGAGGATGAGTCCTAATGAATTGTTGGC
AATCTCAAGGTCTGATTACTATTGTTGGTGCTTAGATAGCAAAGATGTGGCTTAAAATGAAGGGAAGAACAAGGAAAATATTTTA
GTGGGTGAAAAAAAATAGCTAAACTTATTTGAAACTAATAAAATATAAATGGAATAAACATGGATACACATATATGTTTG
SEQIDNO: 271
MEEFEPNHLKTPMVSTSTRHKCAACFQQFKKKENLVEHMKKSYHSVHQPKCGVCHKHCKSFESLREHISGPLAKAHCSSIFAQNGC
HLCLKVFESPVSVDEHKELCLLTAPPPLGTMNIPYTEYEALEDENYMSKYPEAVAINCQMVGCGIDGSVDLLARVCLVDEEDNMIFHT
YVEPQLSVTDYRYEVTGITEEQLKRDGMSVKEVREKISQILYNGESIGKARLDGGKAKLLVGHNLDHALDCLLMNYPENLLRDTATYR
PLLKTNFMSHSLKYLTKTYLGYDIRSGSHDTFQDCVSVMRLYKRMRSQEHQDEETAKQNHLNFDARKSKDHDLQNMSPDELFRISTS
NYQCWCLDSQPRTAEFYGV
SEQIDNO: 272
GGCGATTGTGTTTTGTCCACCGTTACCAACCAGAGCCCTCTCTTGATCAAACCCAAACCCTAATTCCACAAATAAAAACTCCGCT
CTGCATTGTTTACGGCGATTCCGGCGCTTAATTTCTTCTGTTCCCTCCATTTTGAGCCGTTGTGCAATGGAGGAGTTCGAGCCA
AACCATCTAAAAACCCCGATGGTCTCTACAAGCACAAGGCACAAATGTGCAGCATGTTTTCAGCAGTTCAAGAAAAAAGAGAAC
CTTGTTGAACACATGAAGAAATCCTATCACTCAGTGCATCAACCTAAATGCGGTGTTTGTCATAAACACTGCAAATCATTTGAATC
ATTAAGGGAGCATATATCAGGTCCATTAGCCAAAGCTCACTGCTCAAGTATATTTGCACAAAATGGTTGCCACCTTTGTCTGAAA
GTCTTTGAAAGCCCTGTTTCTGTTGATGAGCACAAAGAGTTATGCCTCCTGACTGCCCCTCCCCCCCTCGGGACAATGAATATT
CCCTACACAGAATATGAAGCCTTAGAAGATGAAAACTACATGAGTAAATATCCTGAAGCAGTTGCTATCAACTGCCAAATGGTTG
GGTGTGGTATCGATGGATCAGTTGATCTATTAGCTCGAGTTTGCCTTGTTGATGAAGAAGATAATATGATATTCCACACGTATGT
GGAACCCCAACTATCTGTAACTGATTACAGATATGAAGTAACGGGCATAACAGAAGAGCAGTTAAAAAGAGATGGTATGTCAGT
TAAGGAAGTGAGAGAAAAGATTTCTCAAATTTTGTACAACGGAGAATCAATAGGAAAAGCACGCTTGGATGGTGGAAAAGCTAA
ACTTCTTGTGGGCCATAACCTGGACCATGCTTTGGATTGCTTGTTAATGAACTATCCCGAGAATTTGTTAAGGGATACTGCGACA
TATCGTCCACTACTTAAAACTAATTTCATGAGCCACTCTCTCAAGTACCTCACCAAGACATATCTAGGGTACGATATCAGGTCAG
GGAGTCACGACACGTTTCAGGATTGTGTGTCTGTAATGAGACTGTACAAGCGTATGCGCTCTCAAGAACATCAAGACGAAGAGA
CTGCAAAACAAAACCACCTTAATTTTGATGCTCGAAAATCAAAGGATCATGATCTTCAGAACATGTCACCGGATGAACTCTTTCG
GATTTCAACTTCAAATTATCAATGTTGGTGTTTGGACTCTCAACCACGTACCGCTGAGTTCTATGGTGTTTGAAGAAACCCTAAA
CATATTTATATATCATAAAAGGAAAATGGTTTTGTTAAATATCAAAAGGAATTCACTGAAATAAAATTATACGACTGAATATTTGTA
TTGGTTCTGAAAACTTCAAAAGACGAAAAGAAAATCAGACACGAAGTGCAAGTATCTCTGCCCAAGCTCAAA
SEQIDNO: 273
GCGATTGTGTTTTGTCCACCGTTACCAACCAGAGCCCTCTCTTGATCAAACCCAAACCCTAATTCCACAAATAAAAACTCCGCTC
TGCATTGTTTACGGCGATTCCGGCGCTTAATTTCTTCTGTTCCCTCCATTTTGAGCCGTTGTGCAATGGAGGAGTTCGAGCCAAA
CCATCTAAAAACCCCGATGGTCTCTACAAGCACAAGGTTCTCTGATTTTTATCTCTTCCTTTGTATTCCTTCAATCTCCATCGTTG
CTTATATGCTTTATCAATTTACATTGCTTTCACACTGCATTTGTTTGGTTCCCGGTAATCATGTACGCATGCTAAAATTTATTACTT
TTTGTATCTGATATCATTTTCTTTAATTGGTTGACAACTTGACATATCAAAATGTTCTAAAGCTTTGCCCTTCGCCTACTTGAACTT
TTATCAAGTGTTAAAGCAGTTCACACCATGTTAGTTTATAAGATGGCTATATTAATACCTGTTTTCTCCAGGCACAAATGTGCAGC
ATGTTTTCAGCAGTTCAAGAAAAAAGAGAACCTTGTTGAACACATGAAGAAATCCTATCACTCAGTGCATCAACCTAAATGCGGT
GTTTGTCATAAACACTGCAAATCATTTGAATCATTAAGGGAGCATATATCAGGTGTGATACTTTGAGGGTGTTTGGATGTGCGGT
TTTAAAGTGATCATTGTGATTCAAGAATCAGAATCCGAATTGAAATCACAGTCATTTGATTAAAGTGAGTGATAGTGGCCTTTGGT
TCACAGAATTTGGAATTCAACTCCATTGCACTCCTGGTTTTTGCAATGAGTATGAAACTGAATTCCATTTGAATTTACAAAATCCT
TTGATCATCTGATTTTAATGTCCCATTCCATGGAATTCAATTCCTCGCATTTACTCCTTTTTCAAAATACTCAAATATCCTTTTAAC
CGTAAGTTCAATAGCAAATGTTTCTCACTCATGATTTTTCTTCCATTTTGGGCCAACCGAACTCGTGACACAATTCAATTCCTTCA
TATTCCATTTCCTTTGACAAATTCAAGTTTCTCCCAAAACATTTCTCGAACCAAACTTCCCCTTTTTAATCAGTTTTTTTATGTGCTT
CTGTTTTGTCCTACATAATTAAAATTATAATCATTTGCAAGACACTATGAAACTGTCAAATGGCAATAATCACTTTATCATCGCATC
TAAACACTCCTTTAACATAATCTATCCATTAAATTTTTCATTCTTATTTACATTTTTGTATGGATTATGTAGGTCCATTAGCCAAAGC
TCACTGCTCAAGTATATTTGCACAAAATGGTTGCCACCTTTGTCTGAAAGTCTTTGAAAGCCCTGTTTCTGTTGATGAGCACAAA
GAGTTATGCCTCCTGACTGCCCCTCCCCCCCTCGTAAGTTGATATGGAATAATCTTTTCGATGATGAGGGCATTAACGTCTTTTG
CACAAGCGAGAGATAGAGAGCAAGTCCATGTCCCATCTTTTTTGGTGGACAAGAAATGCAGTGTCATGACTCATGAGTCTCAGT
TTCAATCTAATACACTTGACTTACATTTATTTTATTAATGTCTTGCAGGGGACAATGAATATTCCCTACACAGAATATGAAGCCTTA
GAAGATGAAAACTACATGAGTAAATATCCTGAAGCAGTTGCTATCAACTGCCAAATGGTTGGGTGTGGTATCGATGGATCAGTT
GATCTATTAGCTCGAGTTTGCCTTGTTGATGAAGAAGATAATATGATATTCCACACGTATGTGGAACCCCAACTATCTGTAACTG
ATTACAGGTACTTTTGTCTTCTGTGTTGCATCTTGTTCATTTTTCTTCTAGAATTCGATGACTGCATGTTTATGTGAAAATGGGTCA
ATTCAGGTTATATTTTCTCGATAACAGGTCAAATGGGTTAAACTAAAACATTTTGCTAAAAATGGTAACATGTCAAATGGTTTAAA
AGTTTTACACTTTACAGCCTTCTAAGTTATTTCATTAAAAAAATCGGATATTGCATTTTAATATCGTCCTTGTAATTATATTTCACTT
ACTAATTCTTTACAAATCAGTTTCGGTTTTAGTGAAACTGGTTCATAAAGTTGATGTTTACATGAATTGTGAAATAGATATGAAGTA
ACGGGCATAACAGAAGAGCAGTTAAAAAGAGATGGTATGTCAGTTAAGGAAGTGAGAGAAAAGATTTCTCAAATTTTGTACAAC
GGAGAATCAATAGGAAAAGCACGCTTGGATGGTGGAAAAGCTAAACTTCTTGTGGGCCATAACCTGGACCATGCTTTGGATTGC
TTGTTAATGAACTATCCCGAGAATTTGTTAAGGTTTTCTATATTCCATTTTTTTCTCTTATCAAGTTTTTCTAATATGAAACATGTAT
TCAGTTATAGTATTGTACTTCAGTTTCTGTCTTTTTTTTTTTTTTTAAATCTACCGAATCATAGCAATGAAGATTGCTTTGAATATGA
ATGTCATTCCTATAATTGGGTAGATTTGTCCAAAGTACAAAATGTTGTAATAAGAAGAAATGAAAGTGAATTAATGGAAGTACGTT
GCTATTTCTTGCATCTAACACTTTACATTTACCTTAAAAGATCAGTATTGTTGTAGCAACTTGCCAATTCCTTGCTTTTTATAAAAT
CTAATTATTTCGGCTTTTAATGGTCAAAAAATCTGAATCAGATTTGCCATCGCTACAAAAGTTGGGGGCATCTGCTGCCTAAGCA
TAAATAAATAAATAAATTTAGATTGAGCATGTTTTTGTTCAAATGCTGTTTTTCTGTGTGTAGGGATACTGCGACATATCGTCCACT
ACTTAAAACTAATTTCATGAGCCACTCTCTCAAGTACCTCACCAAGACATATCTAGGGTAAGTTGCTTGATTAGGGTATTTATTTA
TTTTTTATTTTTATATATGAATAATCTTAACACGAATCATGAGTTTTAGCATACTTAATTTTGTTTTATCTGATCCACTTCTACTCAT
GTCACCCAAATATGGGAAATTTTATATTGGTCATGAGCTCTTTTATAGAAAATGGTTTCTAGATTACAAAATGTTCCATCAAATTA
GAAAACCATAAAAATGCCATTCAAAAAAAAAAAAAAAAAACTATAAAAATAGGAAAGTTGGTAGCAAATAATGAAATCTGAGCAAG
TGACCAACAAATAGTTGTGATTTAGTTTTCTAAAATGGGAAGGAAATAGTTACCCCTAAATTCGCCCAAACTAAGCCGTCATTATA
TGTATTATGTAATGTGAGTGGTTTCTAGATTACAAAATATTCCATCAACAAAACCTTACCCATTTGACTTGTCGGAGATAAAAGAA
TAAGATATATCCTATTGAAGCTCATTTTTCATTCTTTCTTTCTGTGTGGGGGTACCACACGTTGTCTGTACATATATTTTTTTTTTTT
AAAAAAAAAAAATTGACCAAAGTTACGACTGTCCTGTTTACTTTGAACTTATGTGATTTTTTTATAACACAACACAAATTTATGTTT
TCAGGTACGATATCAGGTCAGGGAGTCACGACACGTTTCAGGATTGTGTGTCTGTAATGAGACTGTACAAGCGTATGCGCTCTC
AAGAACATCAAGACGAAGAGACTGCAAAACAAAACCACCTTAATTTTGATGCTCGAAAATCAAAGGATCATGATCTTCAGAACAT
GTCACCGGATGAACTCTTTCGGATTTCAACTTCAAATTATCAATGTTGGTGTTTGGACTCTCAACCACGTACCGCTGAGTTCTAT
GGTGTTTGAAGAAACCCTAAACATATTTATATATCATAAAAGGAAAATGGTTTTGTTAAATATCAAAAGGAATTCACTGAAATAAAA
TTATACGACTGAATATTTGTATTGGTTCTGAAAACTTCAAAAGACGAAAAGAAAATCAGACACGAAGTGCAAGTATCTCTGCCCA
AGCTCAAA
SEQIDNO: 274
MRISYHSVHEPTCGICRKHCRSFESLREHLIGPLPKQECRDMFAYRGCKFCLKVFDSPNSRRIHQEKCQHSGTNAGIIGRFSNLGLRD
NLAISGGVRGPQVVALACKMVGGGSDGSLDLCGRVCLIDEHENIIFHSYVKPPIPVTNYRYETTGIRPEYLRDAMPMKQVQRRIQDFL
CNGEPMWTIRARGGRARILVGHGLDHDLECLQIEYRTEKIRDTAKYPPLMKTSKLSNSLKYLTQTYLGYDIQVGIQDPYDDCVATMRL
YMRMRSQIHRTQDYPLVSDPQNRNNFASWRQSELERMTPEQMLEISRSDYYCWCLDSLYP
SEQIDNO: 275
GAATGCTATACCCTACCAACAAACTCATCTGCAAGGTAGAGAGCAGAGGAAGCAAGCATGGCCAAAGATAAAGAAAAAGTAAAA
GGGGAATCCCAAATTAATATACTGAATAATTCTTAGGGAATTGTCCGTTTATGCAAGTTGGGGCAATTCTTACCTCTACCCTTTCT
CTATAAATACCATTCCACTCCACCTTTGACCTCAACTTCAACTCTTTCTTGAAAGTTATACATACATCACTATAAACAAATCATGGA
TTGCAGAATGGAGAGTGCAGAAACTCACAGGTTTTGTTTCTGACATCCATGCACACACACATCACATATACAAGCCTGCATGTG
CATCTTAACTCTCTTAACTCATAGTTAAAACACACATGGATATGTGTAATATATAGCTTTTGTTACTGTAGGAATAAGTGTGCAGC
ATGCTTCAGACAGTTCAACAAACTGGAGCACCTTGTGGAGCACATGAGGATCTCATACCATTCGGTTCATGAACCAACCTGTGG
CATTTGCAGGAAACACTGCAGGTCATTTGAGTCTCTAAGGGAACATCTTATAGGTCCATTGCCAAAGCAGGAATGCAGAGATAT
GTTTGCCTATAGAGGGTGCAAGTTTTGCTTGAAAGTCTTTGACAGCCCTAATTCTCGCAGGATTCACCAAGAAAAATGCCAACAC
TCTGGGACAAATGCTGGAATAATTGGTCGCTTTTCAAACTTGGGACTTCGTGATAATTTGGCCATTAGTGGTGGAGTAAGAGGA
CCACAAGTAGTTGCATTAGCATGTAAAATGGTTGGTGGTGGCAGTGATGGCTCACTAGATCTCTGTGGAAGAGTGTGCTTAATC
GATGAGCATGAGAACATAATCTTCCATTCTTATGTGAAGCCACCAATTCCTGTCACAAACTACAGGTATGAGACCACAGGCATTA
GACCAGAATACCTGAGGGATGCAATGCCAATGAAACAAGTTCAAAGGAGGATTCAAGACTTTCTTTGCAACGGTGAACCTATGT
GGACAATTCGAGCAAGAGGAGGAAGAGCAAGGATTCTTGTGGGCCATGGTTTGGATCATGACCTTGAATGTCTGCAAATAGAAT
ATCGAACAGAAAAGATAAGGGACACTGCAAAATACCCTCCTCTGATGAAAACGAGCAAACTCAGCAACTCGCTCAAGTACTTAA
CACAAACATATCTTGGGTATGACATTCAAGTTGGAATTCAGGATCCTTATGATGACTGTGTGGCAACTATGAGGCTCTACATGAG
AATGAGATCCCAAATACACAGAACACAGGATTACCCTTTGGTGTCTGACCCACAGAACAGGAACAATTTTGCTTCGTGGAGGCA
AAGTGAGCTTGAAAGAATGACTCCTGAACAAATGCTGGAAATATCAAGATCTGACTACTATTGCTGGTGCTTGGATTCCTTGTAT
CCCTGAATATGAGGAAGCAAGTACACAAAATCAGAACCTCATATGAACCACAGGGAATTGGTGAACACAATACCCATTCAACCG
ATATACATCAATAACTACGTACTAAAAGTAACAATAAAGTGCACCAAAGTTGTATCTTTCTACTAGTTAATAATACGCCACTTAAG
CAGAAGCTAGCCTTACACTGCTGTCATAGTGCTAGCACCTTAGAGGCATTGATGTTATGCATGAATATACTTTATTAATACAAAG
AATAATAGAGCTTTCATTAC
SEQIDNO: 276
GAATGCTATACCCTACCAACAAACTCATCTGCAAGGTAGAGAGCAGAGGAAGCAAGCATGGCCAAAGATAAAGAAAAAGTAAAA
GGGGAATCCCAAATTAATATACTGAATAATTCTTAGGGAATTGTCCGTTTATGCAAGTTGGGGCAATTCTTACCTCTACCCTTTCT
CTATAAATACCATTCCACTCCACCTTTGACCTCAACTTCAACTCTTTCTTGAAAGTTATACATACATCACTATAAACAAATCATGGA
TTGCAGAATGGAGAGTGCAGAAACTCACAGGTTTTGTTTCTGACATCCATGCACACACACATCACATATACAAGCCTGCATGTG
CATCTTAACTCTCTTAACTCATAGTTAAAACACACATGGATATGTGTAATATATAGCTTTTGTTACTGTAGGAATAAGTGTGCAGC
ATGCTTCAGACAGTTCAACAAACTGGAGCACCTTGTGGAGCACATGAGGATCTCATACCATTCGGTTCATGAACCAACCTGTGG
CATTTGCAGGAAACACTGCAGGTCATTTGAGTCTCTAAGGGAACATCTTATAGGTAATAATAATCTTTCTTATTAATTGTTGAGCA
AATTGAAGTTGATGGAGTTTCATATTTTTTGAATAGATAATGATCTATTGATTGATGATCATTGTGAATTTTTTATAAATCTGCTTTT
CCAGGTCCATTGCCAAAGCAGGAATGCAGAGATATGTTTGCCTATAGAGGGTGCAAGTTTTGCTTGAAAGTCTTTGACAGCCCT
AATTCTCGCAGGATTCACCAAGAAAAATGCCAACACTCTGGGACAAATGCTGTAAAACTTTTGACTTTGAATTTCCTGTTACATG
ATAAAAAAATATGTGCAGTTAATATACATGGAAACAAGAATTGTATATTTTTTATTATTAATGCTAAAAATTGAAAATGTTTTGGAAT
TATTCATTGCGAATTCCTAACTTTTGGTGCACATTATTACAGGGAATAATTGGTCGCTTTTCAAACTTGGGACTTCGTGATAATTT
GGCCATTAGTGGTGGAGTAAGAGGACCACAAGTAGTTGCATTAGCATGTAAAATGGTTGGTGGTGGCAGTGATGGCTCACTAG
ATCTCTGTGGAAGAGTGTGCTTAATCGATGAGCATGAGAACATAATCTTCCATTCTTATGTGAAGCCACCAATTCCTGTCACAAA
CTACAGGTTGGACAATTAAACTTAGTTTTTGGAGAGTTTAACCTTATATATATATATAGCTGGTGTAGTGAATGGAGCTACTGATT
CATTGACAGGTATGAGACCACAGGCATTAGACCAGAATACCTGAGGGATGCAATGCCAATGAAACAAGTTCAAAGGAGGATTCA
AGACTTTCTTTGCAACGGTGAACCTATGTGGACAATTCGAGCAAGAGGAGGAAGAGCAAGGATTCTTGTGGGCCATGGTTTGG
ATCATGACCTTGAATGTCTGCAAATAGAATATCGAACAGAAAAGATAAGGTAAATCATTCAATTTCAAATTTAGGTTGTCTATCAT
ACATATCTCAGTCAAACATTCTCTTCAGAAATACTTGTAACTTCATTTATGGTAAGTTAAATAACACTTTTCATTAGACCCTACGGG
TTACAGTGACAAAGAAGACATCTTGTTCTTGTTGTATAACTTAATTTGTTGATTTGAAGTGCTTTAATCATCCCTGAAACTAATATA
TGGTGTAGAACAAGCACATATAGGTTTACTACTTTAACACTCATATTCAAATATTGACATAAAAGTCCTTATTGAATAATCAGGGA
CACTGCAAAATACCCTCCTCTGATGAAAACGAGCAAACTCAGCAACTCGCTCAAGTACTTAACACAAACATATCTTGGGCAAGTA
TCTTTTACAATTTTCTTACACAAGATGCAATTTTTATGGAGAATGTTTGAAGATTTTGAGGAACATATGCATGCAGGTATGACATT
CAAGTTGGAATTCAGGATCCTTATGATGACTGTGTGGCAACTATGAGGCTCTACATGAGAATGAGATCCCAAATACACAGAACA
CAGGATTACCCTTTGGTGTCTGACCCACAGAACAGGAACAATTTTGCTTCGTGGAGGCAAAGTGAGCTTGAAAGAATGACTCCT
GAACAAATGCTGGAAATATCAAGATCTGACTACTATTGCTGGTGCTTGGATTCCTTGTATCCCTGAATATGAGGAAGCAAGTACA
CAAAATCAGAACCTCATATGAACCACAGGGAATTGGTGAACACAATACCCATTCAACCGATATACATCAATAACTACGTACTAAA
AGTAACAATAAAGTGCACCAAAGTTGTATCTTTCTACTAGTTAATAATACGCCACTTAAGCAGAAGCTAGCCTTACACTGCTGTC
ATAGTGCTAGCACCTTAGAGGCATTGATGTTATGCATGAATATACTTTATTAATACAAAGAATAATAGAGCTTTCATTAC
SEQIDNO: 277
MRFDLPFDMDAEADSSQNPTPRHKCSACYKQFKKKEHLIEHMKTSNHSVHQPACGVCQKHCKSFESLREHLTGPLPKGVCSKTFS
QRGCQLCLVLFDSPGSLIAHRQTCRLSAPTRLGTKDLTYIDSQFDCQDSSDENHAGRGTGGTVAMDCEMVGGGSDGSLELCARVCL
VDEDENLIFHTYVRPLMTVTNYRYDITGLTEEHLRNAMPLYEVREKVLKILYNGESIGKVRLNGGRAKLLVGHDLAHDLDCLNMNYPD
HMLRDTAKYRPLMKTNLVSHSLKYLTRTYLGYDIQSGTHDPYEDCISVMRLYKRIRAQIHPEEEEDHGTSTPSNRIVGMPDCWRSKEL
DNLTPDQLYAMSRSDYKCWCLDLRSKLPT
SEQIDNO: 278
AAATATTAGTTTAATTGAATGTTAAATTAAAAGTATACCAAATCAGGATTTGATTTGTGATATTTGGTTTGGGGTGACTGCTCCACT
TGCTGCCTACACAGTAGCACGGTGCGTCTTGACTCAAAGCAGTGGTTGCAGCTTCTTCTTTGGTGGTGTTAGGGTTTTGGAGTA
GCCGTTTACACCATCGAAATTTTCGTGTACCTATGTACGACAAAATGTTGTGATTCTTCTATCGACTCCTCTCGCTAACAGGCAA
AATACAAGTCGTTTCATCGTATCTGCGTCACACCAAACCTAGCTGTGCATGCGTTTTGATTTACCATTTGATATGGACGCCGAAG
CGGACTCAAGTCAAAACCCTACTCCAAGACACAAATGTTCGGCATGCTATAAACAATTCAAGAAGAAAGAGCATCTAATTGAGCA
CATGAAAACCTCGAACCATTCCGTCCATCAGCCTGCGTGTGGAGTCTGTCAAAAACACTGCAAATCTTTTGAGTCTCTGAGGGA
ACATCTTACTGGTCCTTTGCCAAAAGGAGTTTGTTCAAAGACTTTCTCTCAACGGGGCTGTCAACTTTGCTTGGTACTATTTGATA
GTCCCGGGTCTCTCATTGCTCACAGACAAACATGTCGCTTGTCTGCTCCTACTCGTCTTGGAACTAAAGATCTGACCTACATAGA
TTCCCAATTTGACTGTCAAGATTCTTCTGATGAAAACCATGCTGGCAGGGGCACTGGAGGAACAGTTGCAATGGACTGTGAAAT
GGTTGGTGGTGGAAGTGATGGTTCTCTGGAACTTTGTGCAAGAGTATGTTTGGTTGATGAAGATGAGAACTTAATCTTTCATACA
TATGTAAGACCTTTAATGACTGTTACTAATTACAGATATGATATAACTGGGTTGACAGAAGAACATCTCAGAAATGCGATGCCCC
TTTATGAAGTTCGAGAAAAGGTATTGAAAATACTATACAATGGAGAATCCATTGGCAAAGTTCGACTAAATGGTGGAAGGGCCAA
GCTTCTTGTGGGGCATGACTTAGCACACGATTTAGATTGCTTGAACATGAATTATCCTGATCATATGCTAAGAGACACTGCAAAG
TACCGTCCGTTGATGAAGACAAACTTGGTCAGCCATTCGCTCAAGTATCTCACCCGAACATATCTTGGTTATGATATCCAATCCG
GCACTCATGACCCGTATGAAGATTGTATTTCTGTGATGAGACTATACAAAAGAATTCGAGCTCAAATTCATCCAGAGGAAGAAGA
AGACCATGGAACATCGACTCCGAGTAACAGGATTGTTGGCATGCCTGATTGCTGGAGATCCAAGGAACTTGACAACCTCACTCC
AGATCAACTCTATGCCATGTCAAGATCGGATTACAAGTGTTGGTGCTTGGATTTGAGGTCAAAATTGCCAACCTGAATTTATTTC
ATCATTAAATTTGTCCGCAGCATCATATCATAATGCTTATTTCTTCAGAAACTATGGGAATGGGCTGCTAAAATATTTTGCAGTGA
TATTTTCAGTGCATATCTTTCTAACGCATCGTGTGATGCTCGGGACAAGAAAACTGTTGAGAAAAAGATAGGTGGTTATGATGCT
TTTGTGAATATCTAACTTTTGGTTCAAACCAAATAAAGGCGACCTTTTTTAGAATCAAGACTTTCCTACCTTTCTCATCATAATAAA
GGCTACCGACACAAACGTAAACTGATTTAAGGCTTGTTCTAGATCCAATTTCAACGGAGCCACTTAGATCTAAGTTGCTGATTGG
TACAAAGAGTTTTCATTTTCAAATATAAACATGTTAAGTTACTATTTAGTTTCCTTTCAAGAAATATATTTTCTTTCTTTACGA
SEQIDNO: 279
AAATATTAGTTTAATTGAATGTTAAATTAAAAGTATACCAAATCAGGATTTGATTTGTGATATTTGGTTTGGGGTGACTGCTCCACT
TGCTGCCTACACAGTAGCACGGTGCGTCTTGACTCAAAGCAGTGGTTGCAGCTTCTTCTTTGGTGGTGTTAGGGTTTTGGAGTA
GCCGTTTACACCATCGAAATTTTCGTGTACCTATGTACGACAAAATGTTGTGATTCTTCTATCGACTCCTCTCGCTAACAGGCAA
AATACAAGTCGTTTCATCGTATCTGCGTCACACCAAACCTAGCTGTGCATGCGTTTTGATTTACCATTTGATATGGACGCCGAAG
CGGACTCAAGTCAAAACCCTACTCCAAGGTCAAACTGCTGCGCTTCACTTTTAATCTTTCTTTTGTTTTTGTTTGTTCCATCATGG
TTGATTACCGTATTGCGTTGATCAGAAAGTCGAACCTTGTAATGAAAGAATTTAGAAGGATTTTAAAATCAAATCCAAATTTATTG
TGTTTTTAAATTAGTTATTACTGCATTTTGTTCTCTGTTGTTTTTTAGAAGATTCTCTTGTGTGAACGAATATTCTGGTTAGTTTTTA
ATCGGTTTGTGGGGACTCTAATGTGTATCTGATATATATAATTGCCTGAGTTTTCTATTTACAGTGACTAATTAGTATCTGCAATAT
AGTGATTGAGGAAATGTTCCGTGCTTCTGAGTAGAATAATATATATTGAACCAGTGCACTAAGTTTTTTTTATTCCTCTGGTATAA
CTGTTCTTTTGCTAGTTTGTTTGAAGAATAGCTGGTTTCTTCCTACAGACACAAATGTTCGGCATGCTATAAACAATTCAAGAAGA
AAGAGCATCTAATTGAGCACATGAAAACCTCGAACCATTCCGTCCATCAGCCTGCGTGTGGAGTCTGTCAAAAACACTGCAAAT
CTTTTGAGTCTCTGAGGGAACATCTTACTGGTAAGTCATTTGTGCATTCAATCTGTGCTGTCGACACTGTTTATTGCTTCCATGTT
TTATGCTCATTGTTTATATTTTCTTGTTCCTTTTTTAACTATGATTTAGGTCCTTTGCCAAAAGGAGTTTGTTCAAAGACTTTCTCTC
AACGGGGCTGTCAACTTTGCTTGGTACTATTTGATAGTCCCGGGTCTCTCATTGCTCACAGACAAACATGTCGCTTGTCTGCTC
CTACTCGTCTTGTAAGTTGATAATTGACAACCTTGAGCATTTATAAAATGAATAGGTTTCTTGTATAATTTGTTATCTATTGGGATT
GAGATTAGGAAGTTTATGTTATGTCTGGAACTGTTGCAGGGAACTAAAGATCTGACCTACATAGATTCCCAATTTGACTGTCAAG
ATTCTTCTGATGAAAACCATGCTGGCAGGGGCACTGGAGGAACAGTTGCAATGGACTGTGAAATGGTTGGTGGTGGAAGTGAT
GGTTCTCTGGAACTTTGTGCAAGAGTATGTTTGGTTGATGAAGATGAGAACTTAATCTTTCATACATATGTAAGACCTTTAATGAC
TGTTACTAATTACAGGTACTTTTAATTTGCTTCTTTGTTTTCTTTTCTTTTACAAATTACGTTTTTTAGGTTTGATAGGGAGAAGGAA
ATGTAAAGGAGGGAGATAGGATAGAAATATCAATGTTTCACTATTTTTTTGGGAGCAGAAATAGGGACGAGGAAGTCTTTCGTAT
GGGACCCTTCTCTCAAATTGGCATGAAATAGGGGGGAGAGATCATGTCTATTTTTCATTTTTTTTTTCGGACTTTCTTGAAAAGTT
AACTAACGTTTTTATGACAAAAAAATATAAATAATAAATAATAAATAATTTAATGTTGAGAATAATAGAGATAAATATATATTTTTTG
GATTAAATTAGTACAAGGATATTTCATATAATTTATTTACTTTTCATTCTCTGTTTCAATCATCTAAACAAATGAGGCAAATTTCTCC
ACTTCTCTTATTTTTATCAACCCAAACAAACAACATATTTTTCAACTACATTTATCTTTATTTCTATCCTTCATATTTCTTTATTCATT
TCTATTCCCAAAATCGCATTATTCTCAATGAAAATTGAGAAAGGGAAAAGAGTGTAGGGGTTGATTTTATTTCCATATTTTCAGAT
ATGATATAACTGGGTTGACAGAAGAACATCTCAGAAATGCGATGCCCCTTTATGAAGTTCGAGAAAAGGTATTGAAAATACTATA
CAATGGAGAATCCATTGGCAAAGTTCGACTAAATGGTGGAAGGGCCAAGCTTCTTGTGGGGCATGACTTAGCACACGATTTAGA
TTGCTTGAACATGAATTATCCTGATCATATGCTAAGGTAGATTCTTCTTGAGTAACTTAAATTGTGCTAAATCTTGCTCGGCAGAA
TTATTCTTGACCACACTCTTGCACTAATTTTTCCACTTTAAGGATTTATCATGATGATGAACTAAACAATTGTTAGAAATCCCTGAA
TTTCTTCTGTTTTTCTCCTTTTTATTATCATCTTATATAAGGTATTATGCTAAACATGTTTCCAGACTTAAAAGATCTATCTATTCAG
AACCTTTTTTCATTATTATATTGGATCATGCCCAGATTGATGGAAATATCTCCTTGAATGTACTGTTTGTTGTTCGAGAGCAGATC
TTTGTAATTATTGCACTCATCTTTTGCCTATGCTTTTATGTTGCATTGAAGAGACACTGCAAAGTACCGTCCGTTGATGAAGACAA
ACTTGGTCAGCCATTCGCTCAAGTATCTCACCCGAACATATCTTGGGCAAGTTAATTCTTAACTCAAATCTTTTTCTGTTTGAAAC
TTCTTCTTCCAGCTTATAGTTTTCCTTGTATTTTAAAAGTCAAATTACAATTGTTCAATGGCTATCATTTCAATTTGCCCAGGATCA
TTGTAGATTCTATGAATGATACTTCTTTCTATACTAAAACAAAATGAAATTGGTGATCAACATTTTTTTATGTTGAGTACGACTAAA
CTAATTTTGAAGGACGTATTCATGTTGATTGTCAAATATAAAATGTTATAAACTAAAACAAGTTTAGTTATAAGGTTTTATTTTTTTG
TGGTTTTTACTCTTCACATTTCGGTACCAAATTTTAAATCCAAACATCCTATCCTGAATCAATGAGACAACAGTGGTGGTGGTGGT
GGTCTGACGGTGCTAACGAGGGAAATTGTGACGACAGTGGTAAACTGGTGGCAATTAGGAGGTGGTGGGTGATAGTTCTAGTG
ATGACAGTTTACATGGATGGTTGAGGAGGGATGCGAGATACTGAATAGCTTGTTCTTCATTTCTTTATACTAAAAGCCATCATAA
AGCAAAACCTTATTTTTAAAGGCAGATAAAAGTATTTCAAAACCAACTAAAAATCAATTCAGCTCCATTTTAGTCAAATGAATTTTG
TTTTGAATTTTTATTTGAAAGTCCAAATTTAAATACTTACCTGTTTAATCTTAAAATTACGATAGTTAACTGATGACAATAATTTTCT
ATTGTTTTTGCTTTACAGTTATGATATCCAATCCGGCACTCATGACCCGTATGAAGATTGTATTTCTGTGATGAGACTATACAAAA
GAATTCGAGCTCAAATTCATCCAGAGGAAGAAGAAGACCATGGAACATCGACTCCGAGTAACAGGATTGTTGGCATGCCTGATT
GCTGGAGATCCAAGGAACTTGACAACCTCACTCCAGATCAACTCTATGCCATGTCAAGATCGGATTACAAGTGTTGGTGCTTGG
ATTTGAGGTCAAAATTGCCAACCTGAATTTATTTCATCATTAAATTTGTCCGCAGCATCATATCATAATGCTTATTTOTTCAGAAAC
TATGGGAATGGGCTGCTAAAATATTTTGCAGTGATATTTTCAGTGCATATCTTTCTAACGCATCGTGTGATGCTCGGGACAAGAA
AACTGTTGAGAAAAAGATAGGTGGTTATGATGCTTTTGTGAATATCTAACTTTTGGTTCAAACCAAATAAAGGCGACCTTTTTTAG
AATCAAGACTTTCCTACCTTTCTCATCATAATAAAGGCTACCGACACAAACGTAAACTGATTTAAGGCTTGTTCTAGATCCAATTT
CAACGGAGCCACTTAGATCTAAGTTGCTGATTGGTACAAAGAGTTTTCATTTTCAAATATAAACATGTTAAGTTACTATTTAGTTT
CCTTTCAAGAAATATATTTTCTTTCTTTACGA
SEQIDNO: 280
MDCRIESAETHRNKCAACFRQFNKLEHLVEHMRISYHSVHEPTCGICRKHCRSFESLREHLIGPLPKQECRDIFSYRGCKFCLKVFDS
PNTRRIHQEKCQHSATNAGIIGRFSNLGLRDNFAISGGARGPQVVALACKMVGGGSDGSLDLCGRVCLTDEHENIIFHSYVKPPIPVT
NYRYETTGIRPEYLRDAMPMRQVQRRIQDFLCNGEPMWTIRARGGRARILVGHGLDHDLESLQIEYRTEKIRDTAKYPPLMKTSKLSN
SLKYDIQAGIQDPYDDCVATMRLYMRMRSQVHRTQDYPLVSDPQNRNNFASWRQSELERMTPEQMLEVSRSDYYCWCMDSLYNP
SEQIDNO: 281
ATGGATTGCAGAATAGAAAGTGCAGAAACTCACAGGAATAAGTGTGCAGCATGCTTCAGACAGTTCAACAAACTGGAGCACCTT
GTGGAGCACATGAGGATCTCATACCATTCGGTTCATGAGCCAACCTGTGGCATTTGCAGGAAACACTGCAGGTCATTTGAGTCT
CTAAGGGAACATCTTATTGGTCCATTGCCAAAGCAGGAATGCAGAGATATATTTTCCTATAGAGGGTGCAAGTTTTGCTTGAAAG
TCTTTGACAGCCCTAATACTCGCAGGATTCACCAAGAAAAATGCCAACACTCTGCAACAAACGCTGGAATAATTGGTCGCTTTTC
AAACTTGGGACTTCGTGATAATTTTGCCATTAGTGGTGGAGCAAGAGGACCACAAGTAGTCGCATTAGCATGTAAAATGGTTGG
AGGTGGTAGTGATGGCTCACTGGATCTCTGTGGAAGAGTGTGCTTAACCGATGAGCATGAGAACATAATATTCCATTCTTATGT
GAAGCCACCAATTCCTGTTACAAACTACAGGTATGAGACAACAGGCATTAGACCAGAATACCTTAGGGATGCAATGCCAATGAG
ACAAGTTCAAAGGAGGATTCAAGACTTTCTTTGCAACGGTGAACCTATGTGGACAATTCGTGCAAGAGGAGGAAGAGCCAGGAT
TCTTGTGGGTCATGGTTTGGATCATGACCTTGAATCTTTGCAAATAGAATATCGAACAGAAAAAATAAGGGACACTGCAAAATAC
CCTCCTCTGATGAAAACGAGCAAGCTCAGCAACTCCCTTAAGTATGACATTCAAGCTGGAATTCAGGATCCTTATGATGACTGT
GTTGCAACTATGAGACTCTACATGAGAATGAGATCCCAAGTTCACAGAACACAGGATTACCCTTTGGTTTCTGATCCACAGAACA
GAAACAATTTTGCTTCGTGGAGGCAAAGTGAGCTCGAAAGAATGACTCCTGAACAAATGCTGGAAGTATCAAGGTCTGACTACT
ATTGCTGGTGCATGGATTCCTTGTATAATCCCTGA
SEQIDNO: 282
ATGGATTGCAGAATAGAAAGTGCAGAAACTCACAGGTTTTGTTTCTGACATCCATGCACAACCACACACATCACATATGCAAGG
GTGTGCATAACTCTTATAACTCATACTTAACACTCACATGGATATGTGTAAGTGACATACACATATTTTGTTACTATAGGAATAAG
TGTGCAGCATGCTTCAGACAGTTCAACAAACTGGAGCACCTTGTGGAGCACATGAGGATCTCATACCATTCGGTTCATGAGCCA
ACCTGTGGCATTTGCAGGAAACACTGCAGGTCATTTGAGTCTCTAAGGGAACATCTTATTGGTAATAATCTTTCTTAATAACTGT
GTTTACATATTCATGGTGAAAATAATTATCACTTCATCATTGTGAATTTTTTATGAGACTCAATCTGCTTTTCCAGGTCCATTGCCA
AAGCAGGAATGCAGAGATATATTTTCCTATAGAGGGTGCAAGTTTTGCTTGAAAGTCTTTGACAGCCCTAATACTCGCAGGATTC
ACCAAGAAAAATGCCAACACTCTGCAACAAACGCTGTACAATTTTTGCCTTTGAATAACTATATATTTTGCAAATGAGATATCCTC
GAAAAACATTATTCTATCTAACTAATGTTTTTCCTGTTAAATGATAAAAATATGTACTGGAGTTAATACATAAAACATGAAATGTAT
ATTCTTTTATTATTAATACTAAAAATTGAAAATATTGTAGAATTATTCATTGGGAATTTCTAACTTTTGGTGAACATTATTATAGGGA
ATAATTGGTCGCTTTTCAAACTTGGGACTTCGTGATAATTTTGCCATTAGTGGTGGAGCAAGAGGACCACAAGTAGTCGCATTAG
CATGTAAAATGGTTGGAGGTGGTAGTGATGGCTCACTGGATCTCTGTGGAAGAGTGTGCTTAACCGATGAGCATGAGAACATAA
TATTCCATTCTTATGTGAAGCCACCAATTCCTGTTACAAACTACAGGTGGGACAATTAAACTTCCTTAGGTTTTGGAGAGTTTAAC
CACATATATATATAGCTGGTGTGGTGAATGGAGCTACTGATTCATTAACAGGTATGAGACAACAGGCATTAGACCAGAATACCTT
AGGGATGCAATGCCAATGAGACAAGTTCAAAGGAGGATTCAAGACTTTCTTTGCAACGGTGAACCTATGTGGACAATTCGTGCA
AGAGGAGGAAGAGCCAGGATTCTTGTGGGTCATGGTTTGGATCATGACCTTGAATCTTTGCAAATAGAATATCGAACAGAAAAA
ATAAGGTAAATCATTCAATTTAACATTTAGGCAGAACATGCAGCATATACGTTGTCTATCATACATATCTCAGTCAAATATTCTCTT
CAGAAATACTTGTAACTTTATTTAAGGAAAGTTAAATAACACTTTTCATTAGACCCAGTGTGTAAAAACTTCACTGTTGACAAAGA
TGACATCTTGTATAATTTATTTTACTGTCACTAAGAGTGTATAACTTAATTTACTTATTTTAAGTTCTTTACTCCTCTCTGAAACAAA
TTATTGGTATAGAACAAGCACATATAGGTTTACTACTTTAATACTCTCTCATATTCAAATATTGACCTAAAAGTCCTTATTGAATAA
TCAGGGACACTGCAAAATACCCTCCTCTGATGAAAACGAGCAAGCTCAGCAACTCCCTTAAGTACTTAACGCAAACATATCTTG
GGCAAGTTTCTTTCACAATTTTCTTCACTGTAAAACACAAGATGCAATTTGATGGATGCCCATCAGCCATAAATGTTTTAAGATTT
TGGGGCACATATATATGCAGGTATGACATTCAAGCTGGAATTCAGGATCCTTATGATGACTGTGTTGCAACTATGAGACTCTACA
TGAGAATGAGATCCCAAGTTCACAGAACACAGGATTACCCTTTGGTTTCTGATCCACAGAACAGAAACAATTTTGCTTCGTGGAG
GCAAAGTGAGCTCGAAAGAATGACTCCTGAACAAATGCTGGAAGTATCAAGGTCTGACTACTATTGCTGGTGCATGGATTCCTT
GTATAATCCCTGA
SEQIDNO: 283
MDAEADSTQIPNIRHKCPACYKQFKKKEHLTEHMKTSNHSVHQPACGVCQKHCKSFESLREHLTGPLPKGVCSKIFSQRGCQLCLVL
FDSPGSLVSHRETCRLSAPTRLGTRELPCIDSQFDCLDSSDENHAGWGPGGAVGMDCEMVGGGSDGSLELCARVCLVDEDENLIF
HSYVQPQIPVTNYRYDITGLTEEHLRNAMPLYEVREKILQMLYNGESIGKVRLNGGKARLLVGHDLAHDLDCLKMNYPDHMLRDTAK
YRPLMKTNFVSHSLKYLTRTYLGYDIQSGTHDPYEDCISVMRLYKRIRAQIHPEEAHETPSHNIVCVPDSWRSRELENLTPDQLYAMS
KSDYKCWCLDLRPRLAS
SEQIDNO: 284
GATTTGATTTGTGATATTTGGTTGGGAGCACTGCTCCACTTGCTGCTTGCACAGTAGCACGGTGCGTTTTGACTCAAAGCAGTG
GTTGCATCTTCTTCCTTCGTGGGGTTAGGGTTTTGGGGTAGCTGAATTTTTCGTGTGCCCGACGATACGAGAAAATGTTGGGAT
TCTTCTATTGACGTTCCATCGTATATGCGCCACACCAAACCACGCTTTGCATGCGTTTGATTTACCATTTGATATGGACGCCGAA
GCGGACTCAACTCAAATCCCTAATATAAGACACAAATGCCCGGCATGCTATAAACAATTCAAGAAGAAAGAGCATCTAACTGAG
CACATGAAAACCTCAAACCATTCCGTCCATCAGCCTGCATGTGGGGTCTGTCAAAAGCACTGCAAATCTTTTGAGTCTCTGAGG
GAACATCTTACTGGTCCTTTGCCAAAAGGAGTTTGTTCAAAGATTTTCTCTCAGCGGGGCTGTCAACTTTGCTTGGTACTATTTG
ATAGTCCCGGGTCTCTCGTTTCTCACAGAGAAACATGTCGCTTATCTGCCCCTACTCGTCTAGGAACACGAGAACTGCCCTGTA
TAGATTCCCAATTTGACTGTCTAGATTCTTCTGATGAAAACCATGCTGGCTGGGGCCCCGGAGGAGCAGTTGGAATGGACTGTG
AAATGGTTGGTGGTGGAAGTGATGGTTCTCTGGAACTTTGTGCAAGAGTATGCTTGGTTGATGAGGATGAGAACTTAATCTTTCA
TTCATATGTACAACCTCAAATACCTGTTACCAATTACAGATATGATATAACTGGATTGACAGAAGAACATCTCAGAAATGCCATGC
CCCTTTATGAAGTTCGAGAAAAGATTTTGCAAATGCTATACAATGGAGAATCCATTGGCAAAGTTAGACTAAATGGTGGAAAGGC
CAGGCTTCTTGTGGGGCATGACTTAGCACACGATTTAGATTGCTTGAAAATGAATTATCCTGATCATATGCTAAGAGACACTGCA
AAGTACCGTCCTTTGATGAAGACAAACTTCGTCAGCCATTCGCTCAAGTATCTCACCCGAACATATCTTGGTTATGATATCCAAT
CCGGCACTCATGATCCGTATGAAGATTGCATTTCTGTCATGAGACTATACAAGAGAATACGAGCTCAAATTCATCCAGAGGAAG
CCCATGAAACACCGAGTCACAACATTGTTTGCGTGCCTGATAGCTGGAGATCCAGGGAACTTGAGAACCTCACTCCAGATCAG
CTCTATGCCATGTCAAAATCGGATTACAAGTGTTGGTGCTTGGATTTGAGGCCTAGATTGGCATCCTGAATTGTTTTATCATTGC
ATTTCTCCACAGCATCATATCATAATATGCATATTTCTTCAGACACTTTGAAAATGGGGTGGTAAAATGTTTTGCAGTGATATTTTC
AGTGCTTATCGCATAGTCTGGTCCTCGGATCAAGAAAAATATTGAGCAAAGACAGGTGTTTGATTATGATGCTTTTGTAAATATAT
CTAACTGTTTGTTCAAACCAAATAAAGGCTACCCTTTTTAGAATGAAGACTTGCCTACCATAATAAAGGCTACCGACACAAACATA
AATGGATGTAAGGTTTGTTCTAGATCCAATTTCAACGGAGCCACTTAGATCTAAGTTGCTGATTGGAAC
SEQIDNO: 285
GATTTGATTTGTGATATTTGGTTGGGAGCACTGCTCCACTTGCTGCTTGCACAGTAGCACGGTGCGTTTTGACTCAAAGCAGTG
GTTGCATCTTCTTCCTTCGTGGGGTTAGGGTTTTGGGGTAGCTGAATTTTTCGTGTGCCCGACGATACGAGAAAATGTTGGGAT
TCTTCTATTGACGTTCCATCGTATATGCGCCACACCAAACCACGCTTTGCATGCGTTTGATTTACCATTTGATATGGACGCCGAA
GCGGACTCAACTCAAATCCCTAATATAAGGTCAATCTGCTGCGCCTCGCTTTCAATTTTTCGTTTGTTTTATTTCTATCATGATTG
ATCACCGTTTTGCCATTGATCGGAAAATGAAATTAGGAGGATGATAGAATCAAATTCAAAGTTATTGAGTTTTTAAATTGGTTATT
TCTGCATTTTATTCTCTGCTTCACTTTAGAGGATTCACTTGTGTGAGAGAATATTCTATCTAGTTTTCAATCGGTTTGTGGGAGCT
AATGAGTATCTCTGAAATATATGATTCCCTGAATTTGCTATTTACAGTGACTAATGCGTATCTGCGATGTGTAGTGAATGATGGAA
TGTACTATGGTTCTGAGTAGAATAATATATATTGAATCAGTGCACTCGGTGTTGTTCTTTTTTTATTCCTTTGCGTAGTTCGTTTGA
AAAATAGTTGATTTTTCTTCTTACAGACACAAATGCCCGGCATGCTATAAACAATTCAAGAAGAAAGAGCATCTAACTGAGCACA
TGAAAACCTCAAACCATTCCGTCCATCAGCCTGCATGTGGGGTCTGTCAAAAGCACTGCAAATCTTTTGAGTCTCTGAGGGAAC
ATCTTACTGGTTAGTCATTTCTGTATTCAATCTGTGCTGTCGACATTATTTATTGATTCCATGTTTTATGCTCGTTGTTTATTTTTTC
TTGTTCCTTTTTTAATTATGTTTTAGGTCCTTTGCCAAAAGGAGTTTGTTCAAAGATTTTCTCTCAGCGGGGCTGTCAACTTTGCT
TGGTACTATTTGATAGTCCCGGGTCTCTCGTTTCTCACAGAGAAACATGTCGCTTATCTGCCCCTACTCGTCTAGTAAGTTGATA
ATTGACAACCTTATGAAAAAATAGCTTTCTTATGTTACCGTTCACCTTAAGTAATCAAAGGAACCTGCTTACATTCAGTGTTTCTT
GTATATTCTTTTATTATCTGTTAGGATTAGGAAGTTTATGTTGTTTGTTGAACTGTTGCAGGGAACACGAGAACTGCCCTGTATAG
ATTCCCAATTTGACTGTCTAGATTCTTCTGATGAAAACCATGCTGGCTGGGGCCCCGGAGGAGCAGTTGGAATGGACTGTGAAA
TGGTTGGTGGTGGAAGTGATGGTTCTCTGGAACTTTGTGCAAGAGTATGCTTGGTTGATGAGGATGAGAACTTAATCTTTCATTC
ATATGTACAACCTCAAATACCTGTTACCAATTACAGGTACTTTTGAATTTGTTTCATTGTTTTCTTTTCTTTTACAGATTACTTGTTT
TAGGTTTGACAGGGAGAAGGAAATGAAAAGGAGGTATATAGGATAGAAATATCAATGTTTCACTATTTTTCTGACAGCAGAAATA
GGGACGAGGGAAGTCTTTCCTATGGGATCCTCCTCTTAAATTGGCACGAAATAGAGGGGAGAGATGATGTCTATTTTTCATTGT
CTTTTTAGTTTCTTAAAAAATTAACTAATATTTGTATAACAAAATAATATAAATAATAAATAATTTAATGTTGAAAATAATAGAGAAAA
ATAATCTTTTTTTGGATTAAATTAGTACAAGGATATTTCATACAATTTATTTACTTTTTCATTCTCTCTTTCACTCATCCAAACAAAT
GAGGCAAATTTCTCCACTTTTTCCTTCTTATTTTTATCAATCCAAGACAACTTATTTTTTTAAATACATTTATCCTTTATTTCTATCCT
TCATATTTCTTTACTTTTATTCCCAATAAAGCATTATTCTCATAGAAAAGTGAGTGTTGGGGTTGATTTTGTTTTGATATTTCAGAT
ATGATATAACTGGATTGACAGAAGAACATCTCAGAAATGCCATGCCCCTTTATGAAGTTCGAGAAAAGATTTTGCAAATGCTATA
CAATGGAGAATCCATTGGCAAAGTTAGACTAAATGGTGGAAAGGCCAGGCTTCTTGTGGGGCATGACTTAGCACACGATTTAGA
TTGCTTGAAAATGAATTATCCTGATCATATGCTAAGGTAGATTCTTCTTAATAACTGAAATTTTGTTAAATCCTGCTTGACAGAATT
ATTCTTGACAACACTGTCTTGCACTAATTTTTTTATCACCGCTTTAAGGATTTATCATGATGATGAACTTAGCAGTTGTTAGAAATC
CCCTGAATTTCTTCTGTTTTTCTCCTTTTTATTATCATCATATATACGGTATTATCCTAAACAAGTTTCCAGACTTTAAAGATCTATA
CTATTCTAATTATTATTAGCACTCATCTTTTGCCTATGCTTTATGTTGTGCATTGAAGAGACACTGCAAAGTACCGTCCTTTGATG
AAGACAAACTTCGTCAGCCATTCGCTCAAGTATCTCACCCGAACATATCTTGGGCAAGTTAATTTTTAACTCAATCTTTTTTCCGT
TTAAAATTTCTTCTTCCAATTTAAAGTTTTCCTTTTATTTTAAAAATCAAATTGGCATGATCTAATCGGCCCCATTTGTATGTACAAT
TGTTCAATGACTACCATTTCAATTTTCCCAGGATCCTTATAGATTCTATGAATGATACTTCTCTCTATACTAAAATAAAATAAAATT
GGAGATTAATACTTTCTTATATTCAGTACGAATAAACTAATTTTGAAGGACATGTTCAAGGTGATTGTAAATATAAAATTTTATAAA
CTAAAACAAGTTTTTAGTTATTTTTTGGTTTCAATTTTTTGTGGTTTCGGTACCGAATTTTAAATCCAAACATCCTATGCTAAATGAT
TGACCACTATCTGGGTCTGTTTGCTGGCAAATGAGGTTTGACTGGCAACAGTTGAAAGTGCAGAGGGGTGATAGTGGAGGTGG
TGGCGGAGGAGGTGTGGTGGTTATAAGTAGTGATGGTAACTGGTGATGAAGTCCGTTGTTGTAGTTGCTGTGGTTATAGTAATG
ACGGTGGTAGCAGTGATAATAAAAACATTGATAGCTGACTGGGGAGCAGCAACAATGAGGACCATGGTGGATTGATTAACGTG
AACAACAGTGGTGGTCTGACAGTGATAACGAGTGTAAAATGGTGACCATAGTGGGAAACTGGTGGCAATGAGGGAGGTGGTGG
GTTCTAGTGATGACAGTTTACACGGATGGTTGAGGAGAGATGGGGAAACTAAATTGCTTGTTCTTCCTACCAAAATCCATCATAA
AGCAAAACCTTTTTTTTTTAAATTCAGATAAAAGTATTTTAAAACCAACTAAAAATCAATTCGGCTCCATTCGAAAATCCAAATTTA
AGAACTTACGAGCATTTTTCTTTTGTTTTAAAACAAAATGTATTTGACTAAAATGAAGCTGCGGAATTGGTTTTTGGCTTTTACTTG
GAAGAACAAGTAATCCAGTTTCTCCCATCTTCCCTTACAAGCATCCAAAGTTACTGCAGTGTGTATGCATCCTCAATCAATACTTA
CCTGTTTAATCTTTAAATTACGGTAGTTAACTGCTCTCAATAATTTTTTGTTGTGTTTTTGCTTTACAGTTATGATATCCAATCCGG
CACTCATGATCCGTATGAAGATTGCATTTCTGTCATGAGACTATACAAGAGAATACGAGCTCAAATTCATCCAGAGGAAGCCCAT
GAAACACCGAGTCACAACATTGTTTGCGTGCCTGATAGCTGGAGATCCAGGGAACTTGAGAACCTCACTCCAGATCAGCTCTAT
GCCATGTCAAAATCGGATTACAAGTGTTGGTGCTTGGATTTGAGGCCTAGATTGGCATCCTGAATTGTTTTATCATTGCATTTCT
CCACAGCATCATATCATAATATGCATATTTCTTCAGACACTTTGAAAATGGGGTGGTAAAATGTTTTGCAGTGATATTTTCAGTGC
TTATCGCATAGTCTGGTCCTCGGATCAAGAAAAATATTGAGCAAAGACAGGTGTTTGATTATGATGCTTTTGTAAATATATCTAAC
TGTTTGTTCAAACCAAATAAAGGCTACCCTTTTTAGAATGAAGACTTGCCTACCATAATAAAGGCTACCGACACAAACATAAATG
GATGTAAGGTTTGTTCTAGATCCAATTTCAACGGAGCCACTTAGATCTAAGTTGCTGATTGGAAC
SEQIDNO: 286
MESSIDASWIHCDNLYKLGLFVYKYHLTPTIYLKNKCAACYRQFNKVEHLVEHMKISYHSVHEPTCGICRKHCRSFESLREHLIGPLPK
QECREIFANRGCKFCLKVLDSPNSRRIHQQKCQLSGLNGISGRFSNMGIRDNLTTGGGATTRGPQVVALACKMVGGGSDGSLDLCG
RVCLIDEHENIIFQCYVNPLIPITNYRYETTGMRQEYLRDAMPLKLVQRKIQDFLCNGEPIWTIRSRGGKAKILVGHGLDHDLASLQIEYP
TTKIRDTAKYPPLMKTSKLSNSLKYDIQNGIQDPYDDCVAAMRLYMRMKTQIHKIEDYPLASDPQNRNNFATWRQSELERMSPEQML
EISRSDYYCWCMDSSYSL
SEQIDNO: 287
ATGGAATCTTCCATTGATGCAAGTTGGATCCATTGTGACAATCTCTATAAACTTGGACTCTTTGTCTATAAATACCATCTCACTCC
TACTATTTACCTCAAGAACAAGTGTGCAGCATGCTACAGGCAATTCAACAAAGTGGAGCACCTAGTGGAGCACATGAAGATCTC
TTACCATTCTGTTCATGAACCAACCTGTGGCATTTGCAGGAAACACTGCAGATCCTTTGAGTCTCTAAGGGAACATCTTATAGGT
CCATTGCCAAAACAAGAATGCAGAGAGATCTTTGCCAACAGAGGGTGCAAGTTTTGCTTGAAAGTCCTTGACAGCCCTAATTCT
CGCAGGATCCACCAACAAAAGTGCCAACTCTCTGGATTAAATGGAATAAGTGGTCGTTTTTCAAACATGGGAATTCGTGATAATT
TGACAACTGGTGGTGGAGCAACAACAAGAGGACCACAAGTAGTTGCACTAGCTTGTAAAATGGTGGGAGGTGGAAGTGATGGG
TCACTAGATCTTTGTGGAAGAGTTTGCTTAATCGATGAGCATGAGAACATCATCTTCCAATGTTATGTCAATCCACTAATTCCAAT
CACAAACTACAGGTATGAAACAACAGGTATGAGACAAGAATACTTAAGGGATGCAATGCCATTGAAACTTGTGCAAAGGAAGAT
TCAAGATTTCCTTTGCAATGGTGAACCCATTTGGACAATTCGTTCAAGAGGTGGAAAAGCTAAGATTCTTGTTGGTCATGGTTTA
GATCATGACCTTGCCTCTTTGCAAATTGAGTATCCAACAACAAAAATAAGGGACACTGCAAAATATCCTCCACTCATGAAAACTA
GCAAGCTCAGCAATTCACTCAAGTATGACATTCAAAATGGGATACAAGATCCTTATGATGATTGTGTTGCAGCAATGAGACTCTA
CATGAGAATGAAAACACAAATACACAAAATAGAGGATTACCCTTTGGCTTCTGATCCACAGAACAGGAACAATTTTGCAACATGG
AGGCAAAGTGAGCTTGAAAGAATGAGTCCTGAACAAATGCTTGAGATCTCAAGATCTGACTACTACTGCTGGTGCATGGATTCC
TCATATAGTCTATGA
SEQIDNO: 288
ATGGAATCTTCCATTGATGCAAGTTGGATCCATTGTGACAATCTCTATAAACTTGGACTCTTTGTCTATAAATACCATCTCACTCC
TACTATTTACCTCAAGTTCAGTTCTCTTCTAGTATTTCTTTCATATCTAAGAGAATTATAAAATTCACTTTAAGGAATTATGGATTGC
AGATTTGAGACTGCAGAAACTCGAAGGTTATCTTTCTTTCTTTTAACCATCTTCGTAGTTTTAAATTGTGATCTGCAATTACGGTT
GCAACAGTCATATTTGTCTGCATTTAGCTAGAGCATAATAGTTTGCAGTGTTCCGCAACTACGATCATAATGGCATATATATGCAT
AATTTATTGAGAATTATATTGTTGTGCATGTGCATCTTAACTAGAATAAAATAACTAATTATATATTTAAAATGGATGTATACGTAAT
TAACATAATACAAAATATAGGAACAAGTGTGCAGCATGCTACAGGCAATTCAACAAAGTGGAGCACCTAGTGGAGCACATGAAG
ATCTCTTACCATTCTGTTCATGAACCAACCTGTGGCATTTGCAGGAAACACTGCAGATCCTTTGAGTCTCTAAGGGAACATCTTA
TAGGTAATTAATTAATTAATTAATTATTCATTGTGAATTTGTCACCATTGAAATTTTTTAAAATGATCTATATATATGCTATTGCAGG
TCCATTGCCAAAACAAGAATGCAGAGAGATCTTTGCCAACAGAGGGTGCAAGTTTTGCTTGAAAGTCCTTGACAGCCCTAATTC
TCGCAGGATCCACCAACAAAAGTGCCAACTCTCTGGATTAAATGTATATATCAATAATCTATCAACTTCATTCTCACTGAACTTCA
ATTAGCATATCTAGTTTCTTATTGGAGGATAATATAGTACTTCCTCCTGTCCTTTTTTATAAGAGAAAGTTGGATTAAAATTAAGTC
AACAAAAAATGATATATGTCCTCCACATTATAAATTAGATATAACTACTTTTGTTGACCATATTTTAACCTAATATGTCTCTTATAAA
AAGGATCAAATGGAGTAATATATATATATATATACATATGAATTCAAAGTTCTTAGAAACAATATTTAGTTAAAAATTTTACTTACCT
CTCGATTAAACTGGAAATTTTTAGACTTCTTATCACTGAAAATTGGAAGGATAATACAAAAAAAAAAAAAAAGCACCTTAAAACCA
ACAATTTAATATTCAATATTTCAAATACATGTTTTGAGTTTCAAACAAAAGTGATATATTTGTAGGGCCGGTAATGGGCATGTGCC
AGCAGACCAACTGCAAGTCCAATTTTTTTTTTTTTTGAAGAGTGTTATTGGATTCAAAATATACTACTAAGAGATAAAAAATCACTT
TGTACTATAGGGATAAAAAATTAAATAAAAAATGCCCATAATTCAAAACTAAAAAAGGTCAAGATTTTTTGACAAAAAATTAATAAG
AAAAATAGAATTAGAGTTTCAATGTTATTGATGATGTTTACAAGTTGTTTTTATGTTATTGATGTTATTTATAATATAAATATAGAAC
GATTATTTTTAAATTTTTTATATTTTTTATTAAGAGCCTAATTTTTAAAATTAAAACAGAACCTCTAAAATGTACGATACATTCTTGTA
TATTTAGTTTAATATTTAAAACCATACATCGATTTTATTTGATACACTTTGTCTATATTTAAGGTTTGAGGGAGTAGAGTAGTGATA
TCATATTAATTAATACTATGAGTTGGTTTTGGAGTAATGAAGCTGATTCTCACTCTTATATATAGGGAATAAGTGGTCGTTTTTCAA
ACATGGGAATTCGTGATAATTTGACAACTGGTGGTGGAGCAACAACAAGAGGACCACAAGTAGTTGCACTAGCTTGTAAAATGG
TGGGAGGTGGAAGTGATGGGTCACTAGATCTTTGTGGAAGAGTTTGCTTAATCGATGAGCATGAGAACATCATCTTCCAATGTT
ATGTCAATCCACTAATTCCAATCACAAACTACAGGTATGAAACAACAGGTATGAGACAAGAATACTTAAGGGATGCAATGCCATT
GAAACTTGTGCAAAGGAAGATTCAAGATTTCCTTTGCAATGGTGAACCCATTTGGACAATTCGTTCAAGAGGTGGAAAAGCTAA
GATTCTTGTTGGTCATGGTTTAGATCATGACCTTGCCTCTTTGCAAATTGAGTATCCAACAACAAAAATAAGGTAAATTATATTCA
CTCTAAAATACTTGAGCGTGCACATTCGCTACTGAATTTGGATCATCTAGGACTGTAACATTAGTTAAAAAAAAAAAATATTAAAT
CTAAGTTTAGTAAGAGTTAAAAATTTATACACTTAAATTCAACGACTCTAAAATAATTATATAACTTAGATCTTCAATACACATTGAT
ATGTCTCCATAATAATTACATAAAAGTGGAAGAATATGTAATTCTAAGGATAAAATCTTTTTAAAAAATTGAATAGACCAAAATCAT
ATTTATATTAGAAGTACCAAAAGTGTGATTAACTTATTAATTATTATGTCAACAGGGACACTGCAAAATATCCTCCACTCATGAAA
ACTAGCAAGCTCAGCAATTCACTCAAGTACTTAACACAAGTATATCTTGGGCAAGTATCATTTGAATTTCATTTTATTTTATTGAAA
AAAAATGTTAAATGTCAATTTGTCAAACTTACATACATATAATATCTATTTCAAATAATTAGACATATAATTTGGTGCACATATATTA
TACATACAGGTATGACATTCAAAATGGGATACAAGATCCTTATGATGATTGTGTTGCAGCAATGAGACTCTACATGAGAATGAAA
ACACAAATACACAAAATAGAGGATTACCCTTTGGCTTCTGATCCACAGAACAGGAACAATTTTGCAACATGGAGGCAAAGTGAG
CTTGAAAGAATGAGTCCTGAACAAATGCTTGAGATCTCAAGATCTGACTACTACTGCTGGTGCATGGATTCCTCATATAGTCTAT
GA
SEQIDNO: 289
MESAESLRNKCRACYRQFNKMEHLVEHMRTSYHSGHEPMCGICKKHCRSHESLREHLIGPLPKVECERIFKERGCHICLDIFSSRNA
LKAHLNSCQLSRTNVNGLTYRMANLGIFDDMRNHENGGGRGGRVVALACKMVGGGSDGSLDLCARVCLIDEYENTIFHTYVRPHLP
VTHYRFETTGIRPEMLKDAMGVRQVQKKIQDFLSNGEPLWKIRPKGGKARILVGHGLDHDLKCLEVDFPGMMIRDTAKYPPLMKTSK
LSNSLKYLAKAYLGYDIQTGIQDPYEDCVATMRLYKRMKYQRHKIEDFPQVTDPHRNNFASWRQNELERMSPDDLLALSRSDYYCW
CLDSRDI
SEQIDNO: 290
ATGGAGTCTGCCGAATCTCTAAGGAACAAGTGCCGGGCATGCTATAGACAGTTCAACAAAATGGAGCACCTAGTGGAACACAT
GAGGACCTCATACCATTCAGGCCATGAACCCATGTGTGGCATTTGTAAGAAACATTGCCGTTCACACGAATCTCTCAGGGAACA
CCTAATAGGGCCATTGCCAAAAGTAGAATGTGAAAGGATATTTAAAGAGCGTGGATGTCACATTTGTCTTGACATATTTAGCAGC
CGTAATGCTCTTAAAGCACACCTTAATTCATGTCAGCTCTCACGCACAAATGTCAATGGCTTAACATATCGCATGGCAAACTTAG
GAATTTTTGACGACATGAGAAATCATGAAAATGGAGGTGGAAGAGGAGGAAGAGTGGTTGCACTAGCTTGCAAGATGGTTGGT
GGTGGGAGTGACGGCTCCTTGGATCTTTGTGCAAGGGTTTGTCTTATTGATGAATATGAAAACACTATCTTCCATACCTATGTCA
GGCCTCACCTTCCTGTCACTCACTACAGGTTTGAAACAACGGGGATACGACCAGAGATGTTGAAAGATGCAATGGGAGTGAGG
CAAGTCCAAAAAAAAATTCAAGACTTCCTGAGCAATGGAGAACCATTATGGAAGATCCGTCCCAAGGGTGGAAAAGCGCGAATT
CTTGTTGGTCACGGTTTAGATCATGATCTCAAATGTTTAGAGGTGGATTTTCCAGGAATGATGATTAGAGATACAGCAAAATATC
CTCCATTAATGAAAACCAGCAAGCTCAGCAACTCACTCAAGTACCTAGCAAAAGCATATCTTGGTTACGACATTCAAACAGGTAT
ACAAGACCCTTATGAAGACTGTGTGGCAACAATGAGGCTGTACAAGCGAATGAAGTACCAACGTCACAAAATTGAGGACTTCCC
TCAAGTTACTGACCCTCATCGAAATAATTTTGCTTCATGGAGGCAAAATGAGCTTGAAAGGATGAGTCCAGATGACCTGTTAGCA
CTTTCAAGGTCCGATTACTATTGCTGGTGCTTGGATTCTCGGGATATATGA
SEQIDNO: 291
ATGGAGTCTGCCGAATCTCTAAGGTTCTTATCATTCTCCTCCTCTAATGCTGTATATAATGGTTAAATTACGGCAAAGGTGATCCT
AATTAAGTCATGTGATTTCAGGAACAAGTGCCGGGCATGCTATAGACAGTTCAACAAAATGGAGCACCTAGTGGAACACATGAG
GACCTCATACCATTCAGGCCATGAACCCATGTGTGGCATTTGTAAGAAACATTGCCGTTCACACGAATCTCTCAGGGAACACCT
AATAGGTATCTATGTAAAACACATACAGATATTTATTCTCTTTCATTTTTATCTTGCCTTGTTAAGTTCCTATGCAGTGATACATCTT
TTTAAATTCTTGATTAACCTGTGATCTCAGGGCCATTGCCAAAAGTAGAATGTGAAAGGATATTTAAAGAGCGTGGATGTCACAT
TTGTCTTGACATATTTAGCAGCCGTAATGCTCTTAAAGCACACCTTAATTCATGTCAGCTCTCACGCACAAATGTCAATGTAAGTT
GTATATGCTATGAACTAATCATTCTTGTAGCTTTGCTATGCACAAAAATAGTATCAAATTAACCATATGTAACTAATTTACTTGCTA
AATTCTTTCAGGGCTTAACATATCGCATGGCAAACTTAGGAATTTTTGACGACATGAGAAATCATGAAAATGGAGGTGGAAGAG
GAGGAAGAGTGGTTGCACTAGCTTGCAAGATGGTTGGTGGTGGGAGTGACGGCTCCTTGGATCTTTGTGCAAGGGTTTGTCTT
ATTGATGAATATGAAAACACTATCTTCCATACCTATGTCAGGCCTCACCTTCCTGTCACTCACTACAGGTAATTAAGCATAAATAT
GAAGTTTTTGTGACTATCTGATCAACAAGAAAATAAAAACTATATTCGTACAATTTGTGTGTGTTTCTGTTAATGAAAAATGATGG
GATATTATTAGGTTTGAAACAACGGGGATACGACCAGAGATGTTGAAAGATGCAATGGGAGTGAGGCAAGTCCAAAAAAAAATT
CAAGACTTCCTGAGCAATGGAGAACCATTATGGAAGATCCGTCCCAAGGGTGGAAAAGCGCGAATTCTTGTTGGTCACGGTTTA
GATCATGATCTCAAATGTTTAGAGGTGGATTTTCCAGGAATGATGATTAGGTAAAGTACTCCATATATTTCACAAACACAGAAACA
CAAGTATTGTAACTTTGCACAATTCAATATCTAGACACAAATAAATAACATAAGTTACCTTTGATTTTTGGCGCAGAGATACAGCA
AAATATCCTCCATTAATGAAAACCAGCAAGCTCAGCAACTCACTCAAGTACCTAGCAAAAGCATATCTTGGGTAACTTCCATAAT
TACTCAATCAGTATATGCAACTCTTGTTGTTTCTACAAAAAAAAAACACAAAAGTATAAAGCATAGGCGGTCATTATAGAAATTTT
AACTTGCAAACATTTCCTCAGGTAGTTGATGGTTAATCATCAACCTTTGCTAATTTAAATAGTATTATTTTTCTCTTCAGAAAATGT
TATTTAGATACTGAAGCATCTAGCTCCACCGAAACATCATTCTCATGTTAGGTAGTACTTCAGAATATATGTGCAACTACAAACCA
GGGGATATGATGTTCTTCTTAAGTTTATTATGGAGTACAGTAGAAAATTAAATTTAATTATTTTCTCTTTCTCCTTTTTCAGTTACG
ACATTCAAACAGGTATACAAGACCCTTATGAAGACTGTGTGGCAACAATGAGGCTGTACAAGCGAATGAAGTACCAACGTCACA
AAATTGAGGACTTCCCTCAAGTTACTGACCCTCATCGAAATAATTTTGCTTCATGGAGGCAAAATGAGCTTGAAAGGATGAGTCC
AGATGACCTGTTAGCACTTTCAAGGTCCGATTACTATTGCTGGTGCTTGGATTCTCGGGATATATGA
SEQIDNO: 292
MESAEHLRNKCAACYRQFNRMEHLVEHMRTSYHSDHEPMCGICRKHCRSFESLREHLIGPLPKAECERIFKERGCHICLNILGSRNA
LRSHLNACQLPRTNVNGLAYRMANLGIYDNTRNYENAGGRVVALACKMVGGGSDGSLDLCARVCLIDEYENIIFHTYVKPQLPVTHF
RYETTGIRPEMIMDAPGVRQVQRKIQEFLCNGEAMWKIRPKGGKARILVGHGDTAKYPPLMKTSKLSNSLKYLAKAYLGYEIQTGIQD
PYEDCVATMRLYKKMKYQSHNNKVEDIPLATENRNNFASWRSNELERMSPDELLRISASDYYCWCLDS
SEQIDNO: 293
ATGGAATCTGCAGAGCACCTGAGGAACAAGTGTGCAGCATGCTACAGACAATTCAACAGGATGGAGCACTTGGTGGAACACAT
GAGGACCTCGTACCATTCTGACCATGAACCCATGTGTGGTATCTGCAGGAAACATTGTCGATCATTTGAATCCCTCAGGGAGCA
CCTTATAGGACCATTGCCAAAAGCCGAATGTGAAAGGATATTCAAAGAGCGTGGATGTCACATCTGTCTGAACATTCTTGGCAG
CCGCAATGCTCTCAGGTCTCACCTTAACGCATGCCAGCTCCCACGCACCAACGTAAATGGGTTGGCATATCGCATGGCAAACTT
GGGGATTTACGACAACACAAGAAACTATGAGAATGCAGGTGGGAGAGTGGTGGCACTTGCTTGCAAGATGGTTGGTGGGGGG
AGTGATGGCTCCTTGGATCTCTGTGCTAGGGTTTGTCTTATTGATGAATATGAGAACATTATOTTCCATACCTATGTCAAACCACA
GCTTCCTGTCACTCACTTTAGGTATGAAACAACAGGCATACGACCAGAGATGATTATGGATGCACCAGGAGTGCGGCAAGTTCA
AAGGAAGATTCAAGAATTCCTCTGCAATGGAGAAGCGATGTGGAAGATCCGGCCTAAGGGAGGAAAAGCAAGAATCCTTGTTG
GTCACGGAGATACAGCAAAATATCCTCCACTAATGAAAACCAGCAAGCTCAGCAACTCACTCAAGTACCTAGCAAAAGCATACC
TCGGTTACGAGATTCAAACAGGTATACAAGATCCTTACGAGGATTGTGTGGCAACAATGCGGCTATACAAGAAAATGAAGTACC
AGTCTCATAACAATAAAGTAGAGGACATCCCTCTTGCCACTGAAAACCGCAACAATTTTGCTTCATGGAGGTCGAATGAACTCGA
GAGGATGAGTCCGGATGAACTCCTGCGGATCTCCGCATCTGATTACTACTGCTGGTGCTTGGATTCTTAA
SEQIDNO: 294
ATGGAATCTGCAGAGCACCTGAGGTTCTTAATTCTTCTTTTTGTCCATCGCTTGACTTCTATGCATAACTAATAGACATGATGATC
AGTATCTAGTTACAATTTAATTGCAGGAACAAGTGTGCAGCATGCTACAGACAATTCAACAGGATGGAGCACTTGGTGGAACAC
ATGAGGACCTCGTACCATTCTGACCATGAACCCATGTGTGGTATCTGCAGGAAACATTGTCGATCATTTGAATCCCTCAGGGAG
CACCTTATAGGTATCCATTTTGATCACTCAAAGCATACATGCCTGCTCTATACTCCAATACACTCATAGGTATCCTTTTGTAGTTG
CAGATTTGTTAATTTTTCTTCATTTACTCAATTTCTCATATGCAGGACCATTGCCAAAAGCCGAATGTGAAAGGATATTCAAAGAG
CGTGGATGTCACATCTGTCTGAACATTCTTGGCAGCCGCAATGCTCTCAGGTCTCACCTTAACGCATGCCAGCTCCCACGCACC
AACGTAAATGTAAGCCTGTAAATGAAAATGAAACAATTTGTTTTTCCATCAATTGACTGCTGATCACATGGATTATTTACACTCATT
AACCGCAGGGGTTGGCATATCGCATGGCAAACTTGGGGATTTACGACAACACAAGAAACTATGAGAATGCAGGTGGGAGAGTG
GTGGCACTTGCTTGCAAGATGGTTGGTGGGGGGAGTGATGGCTCCTTGGATCTCTGTGCTAGGGTTTGTCTTATTGATGAATAT
GAGAACATTATCTTCCATACCTATGTCAAACCACAGCTTCCTGTCACTCACTTTAGGTACCTATTTTCAAGCTTCATCTGCTTCTA
TACGACTCCCATCTATCCCAAAAAATGGCTTGGTTTTTCCAATCAAATAACCAGTTTTGTAATGCAAAATCTTCATTTATATGATTT
TTTTTATTAAAAATTATATGGAATAACAATATGGTGTCAATATGTTTACAATTTTATGTGACAGTTAGAAAAAACGAAAATTTTCAG
GTATGAAACAACAGGCATACGACCAGAGATGATTATGGATGCACCAGGAGTGCGGCAAGTTCAAAGGAAGATTCAAGAATTCCT
CTGCAATGGAGAAGCGATGTGGAAGATCCGGCCTAAGGGAGGAAAAGCAAGAATCCTTGTTGGTCACGGGTTGGATCACGATC
TCAGATGCTTAGAGGTGGACTACCCTGGAACAATGATCAGGCAAGTTTTATCTCACATTCACATATATAAACCATCCACAGTACA
TAACTAAACATTAGAACGATTAAAACCTGAACTTGATGAGCACGATTGGATAGCCCAAGTGGTGGGTTTATCCTTTGTTGTCCCC
AAACACTCGGGTTCAACTCTGGCTCATCCTAAGAAGTATTAAAACAGATATTCATTTGCAAGGCATATAAGCCTAAATTGTCAAC
AAAAAAAAACCTGAACTTGATTAACAAGTCATTCTTCTGTTTAATTTGGTTGCAGAGATACAGCAAAATATCCTCCACTAATGAAA
ACCAGCAAGCTCAGCAACTCACTCAAGTACCTAGCAAAAGCATACCTCGGGTAAACTACTTAATAAATTCACACTATATCGTTTC
GCTCTTCTGCCTCTGCATACTGAACACATTTAATCTCGGCCAATCATTCACTTTATTTGTGTTATCAGTTACGAGATTCAAACAGG
TATACAAGATCCTTACGAGGATTGTGTGGCAACAATGCGGCTATACAAGAAAATGAAGTACCAGTCTCATAACAATAAAGTAGAG
GACATCCCTCTTGCCACTGAAAACCGCAACAATTTTGCTTCATGGAGGTCGAATGAACTCGAGAGGATGAGTCCGGATGAACTC
CTGCGGATCTCCGCATCTGATTACTACTGCTGGTGCTTGGATTCTTAA
SEQIDNO: 295
MSRLGLQSSSDFDARAHGARVVALGCKMVGGGSDGTLDLCARVCLIDENENIIFHTYIRPRLPVTNYRYETTGIRPENLRDAIPLKLAQ
RKIQDFLCNGEALWKIRSQGGRARILVGHGVDHDLGCLGIEYPSLLIRDTASYPPLMKTSMLSNSLKYLTHAYLGYEIQNGIQDPYEDC
VAAMRLYVRMRSQAHPRDYSSGSGEAQSRNKFPSWRQRELERMTPEALLEISASDYYCWCLDS
SEQIDNO: 296
ATGTCGAGATTGGGTTTACAAAGCTCATCAGACTTTGATGCCAGAGCTCATGGAGCCAGAGTGGTGGCATTGGGTTGCAAAATG
GTCGGAGGAGGAAGTGATGGAACATTAGATCTCTGTGCTAGGGTTTGCCTCATTGATGAAAATGAAAATATCATCTTCCACACTT
ACATCAGACCTCGGCTACCAGTCACAAACTACAGGTACGAAACAACGGGAATAAGGCCCGAAAACTTGAGGGATGCAATCCCA
TTGAAGCTAGCTCAAAGGAAGATTCAAGATTTCTTATGCAATGGGGAAGCACTATGGAAAATCCGATCACAGGGTGGAAGGGCA
AGGATTCTGGTGGGACATGGAGTAGATCACGATTTAGGATGTCTAGGAATTGAATACCCTTCACTTCTTATCAGGGACACTGCC
AGTTATCCCCCACTGATGAAAACAAGCATGCTTAGCAACTCACTCAAGTATCTCACACACGCCTACCTTGGTTATGAAATTCAAA
ATGGCATACAAGATCCATATGAGGACTGTGTAGCAGCCATGAGGCTATATGTCAGAATGCGCTCACAGGCCCACCCACGTGAC
TATTCCTCAGGATCAGGTGAAGCTCAAAGCCGTAATAAATTTCCATCTTGGAGACAGAGAGAGCTTGAGAGGATGACTCCAGAG
GCCCTGCTGGAGATTTCTGCATCTGACTACTACTGTTGGTGCTTGGACTCTTAA
SEQIDNO: 297
ATGTCGAGATTGGGTTTACAAAGCTCATCAGACTTTGATGCCAGAGCTCATGGAGCCAGAGTGGTGGCATTGGGTTGCAAAATG
GTCGGAGGAGGAAGTGATGGAACATTAGATCTCTGTGCTAGGGTTTGCCTCATTGATGAAAATGAAAATATCATCTTCCACACTT
ACATCAGACCTCGGCTACCAGTCACAAACTACAGGTAACAAAAGAAAATTTCTGAACTGGATAGTACTGGTAATCTTCTTGAAGT
GGTTTGTATTCCTGATACATCTGAATTTTGGACAACTTCATAGGTACGAAACAACGGGAATAAGGCCCGAAAACTTGAGGGATG
CAATCCCATTGAAGCTAGCTCAAAGGAAGATTCAAGATTTCTTATGCAATGGGGAAGCACTATGGAAAATCCGATCACAGGGTG
GAAGGGCAAGGATTCTGGTGGGACATGGAGTAGATCACGATTTAGGATGTCTAGGAATTGAATACCCTTCACTTCTTATCAGGT
AATTGAGGTTCTTATACATCAAATATTCTTGCAAACCTAATGATAGTTTTTTGTTTTGCATTTACATAGTTGCCTTCTCACAGGGAC
ACTGCCAGTTATCCCCCACTGATGAAAACAAGCATGCTTAGCAACTCACTCAAGTATCTCACACACGCCTACCTTGGGTAAGTA
CTAAGTAGTAAGTACATTATGATTTTCTTCATCATTTAAGTTTACACCCTAGAATAACTCAAAACAACTAAATTCAATTACATCTTG
ATTGGTCCACCAACCCTGATGCATATATATAAAATTTGTTTCTTTCCGTTTCAAATGTGTGCACCATGATAAGCAGTGTGGAATAT
ATAAAAATACAACCATTTTTACATCGGTTATCATATCCATATTATACTCGAGCAAATACTCACTTGAATTTACCCCTCCCTTTCGAA
TGACTAGTTATGAAATTCAAAATGGCATACAAGATCCATATGAGGACTGTGTAGCAGCCATGAGGCTATATGTCAGAATGCGCT
CACAGGCCCACCCACGTGACTATTCCTCAGGATCAGGTGAAGCTCAAAGCCGTAATAAATTTCCATCTTGGAGACAGAGAGAG
CTTGAGAGGATGACTCCAGAGGCCCTGCTGGAGATTTCTGCATCTGACTACTACTGTTGGTGCTTGGACTCTTAA
SEQIDNO: 298
MEPQAEIPKSSRHKCSACFKQYKIKDHLVEHMKKSYHSVHQPKCGVCKKHCKTLESVREHLTGPLAKSDCAVVFAALGCKLCLSIFD
NVDARGLHEVSCSFDPPSVPELMSLPIADMCDEVEAAVLNSAVPDDNGTCPKLVALDCEMVGGGDYGSFNLCGRVCLVNEKEEVIF
HAYVKPIIPITDYRYELTGITEDHIVDATPICEVSKRVKEILYNGEESTWRMRTDGGEACLLIGHDLDHDLEVLRMDYPENLTRDTAKYR
PLLKTNLKSHSLKYLTKTYLGYEIQSGTHNPYEDAVSAMRLYKRIRSIVHQTNEISIQDNDDSIYYTNDSKKNSPFDSNKNNKKKSPFDL
YKKCELLAKSPDELLEISKSSYKCWCWDAKRTLNRMIEE
SEQIDNO: 299
ATGGAACCACAGGCAGAAATTCCCAAATCCTCGAGGCACAAATGCTCCGCATGTTTCAAGCAATACAAGATAAAAGATCACCTT
GTCGAACATATGAAGAAATCATATCATTCAGTTCATCAGCCCAAGTGTGGGGTCTGTAAAAAGCATTGCAAGACACTGGAATCA
GTTAGAGAGCATCTTACAGGTCCACTGGCCAAATCTGACTGTGCAGTGGTCTTTGCTGCCTTGGGTTGTAAGCTCTGTCTATCA
ATATTTGATAATGTTGATGCTCGTGGCCTACATGAAGTCTCTTGTAGCTTTGATCCACCTTCTGTCCCTGAGCTAATGTCCTTGC
CTATTGCTGACATGTGCGATGAAGTTGAAGCGGCAGTTTTGAACTCAGCTGTGCCAGATGATAATGGTACTTGCCCCAAATTAG
TCGCTTTGGACTGTGAAATGGTTGGAGGTGGAGATTATGGTTCATTTAACCTTTGTGGTAGAGTTTGCCTTGTTAATGAAAAGGA
GGAGGTGATATTTCATGCATATGTAAAGCCTATAATTCCAATCACTGATTACAGATATGAATTAACTGGCATAACAGAGGATCATA
TCGTAGATGCAACACCAATTTGTGAAGTTTCAAAGAGAGTCAAAGAAATATTGTATAATGGGGAAGAATCGACATGGAGAATGC
GAACTGATGGTGGAGAAGCTTGTCTTCTTATAGGTCATGACTTGGACCATGATCTTGAAGTTTTACGAATGGACTACCCTGAGAA
CTTGACACGGGATACAGCCAAATACCGTCCGCTTCTGAAAACAAATTTGAAAAGCCATTCTCTGAAGTATCTGACCAAGACCTAC
TTGGGGTATGAGATACAGTCTGGTACACATAATCCATATGAGGATGCAGTTTCTGCTATGAGACTTTATAAAAGGATTCGCTCTA
TTGTGCACCAAACCAATGAAATTTCCATTCAAGATAATGATGATAGCATCTATTATACCAACGACAGCAAGAAGAACAGCCCATT
TGATTCCAACAAGAACAATAAGAAGAAAAGCCCATTTGATCTATACAAGAAATGTGAACTCTTGGCTAAATCCCCGGATGAGCTA
CTTGAGATATCCAAGTCCTCCTACAAATGTTGGTGCTGGGATGCAAAAAGAACGCTAAACAGGATGATAGAAGAGTGA
SEQIDNO: 300
ATGGAACCACAGGCAGAAATTCCCAAATCCTCGAGGTGAATATTTCCTTTTATCTTCTAATTCTGTAGCTATCCATTAATTTCTTTT
GTCATTCAATCCTGTATTTGGTAATTAGAGTGAAAATTAAATGACTTCTTGGGTTGCTTACCTACTTACCGCATTTCCTTTTAAGG
TGATGCATGGGTTTGTTGCTTATCGGTTTTTAAATTCAAATTTATGTGGAAAACTCAAATGGGTTTTGATTTTTTCAGTGATCTTAG
GAATCATACTTCGACCTGCTAGTTAAGGGGGGGTTAGTCATCTAGAACTTAAATTTGGAACTGGGATAATCTGTGTGATAAATTT
TTAAGGTGTTAGTCACATAGAACTAAAACTAGAATAAAATTTTACGGTGTTAGTCAAGTAGAACTCTAACAAAACGAGAATTGGG
ATAATGGTACAAAATTATCCCATTTTTTTATTTTTTTATACAAAAATAAGAGTTCTAGTCTCTCAAATTATTCTAAGTTTTAGCTTGG
AACTAGGTGGTAAAAAACTAAAACTCATATTTATTTTGTTTTTTTTTTATTTTTATATTTTTTCCTTTAGGGGGGGTTAGTCATCTAG
AACTTAAATTTGGAACTGGGATAATCTGTGTGATAAATTTTTAAGGTGTTAGTCACATAGAACTAAAACTAGAATAAAATTTTACG
GTGTTAGTCAAGTAGAACTCTAACAAAACGAGAATTGGGATAATGGTACAAAATTATCTCACTTTTTTTATTTTTTTATACAAAAAT
GAGAGTTCTAGTCTCTCAAATTATTCTAAGTTTTAGCTTGGAACTAGGTGGTATAAAACTAAAACTCATATTTATTTTGTTTTTTTTT
ATTTTTATATTTTTTCTTTACTTCTTTCCTATTATGGGTATCACTTAAATGTCCCAATTAACTAACACCCCCTAAGGGAGTIGTTTC
CAATTTAGAGAAAAACCTGGACGCTTTTAGGTATTTCAGTGCATTTATTCTTGTTATAGTTTTAGGGACTTTTTTTAAATTAGTCTT
TGAAGGGCATTGAATATTAGCGATTAACCCATCCTTGTTTAGTTTTAAATTATAAAATTTTAAGAGAAGTAAACAACGCGGTTAAC
CCCCCCCCCCTAAGCTCCGATTCCCGCTACTCCACTCTAGCCACATGGTGCTTACAAGTGCAGACAATTCCTGTACCTTTATTG
TCAGGGGGAACCCCTTTTGATGCCTCAACAGCTCACGTAGGCACCTTAGGCACACCAAAGGCTTGTGGGACATGACATGCAAG
GTGCCCTTACCTCTATTTTGAAATTTTGAGAGTGCGACGGGTGTATAACAAAGTAAACTGATTTTGGAAATAAAATATATATCTTT
TCTTTTCCTTGGCAGATTGTTTGATATTTTTTCTTGTAATGTGCCTAATTACCTATACCTATATTCGAGGACATGACAGTTTGAAAC
TTGAAGTCCAAACTTTATTGCTTTATGACCTATGTAACTAATTAGTAGTTAGTAGAACTATTTAATATTTGTTACTACTCTGTTTAGA
TGGTTCGTGTGTTGTTAAATATCATAGTTTGTGACTTTTTGTGCAATTTTTGAAACTTTTTGGATTTTTTAATAATACTTCATGAATT
ATAATATGTTAGCTATGTATGATTTCTTAATCTTATTTTTTTAAATTGTTGAGTTGTAGGTAGTTCACGCAGGATTAATATAGGATA
TAATTAGTATTAGGATAGTGTAAGATTTAAATGAAATATTCAAAATAATGTTTCATGTATTTTATTAATCTTTTTTTATAAATTGTTGA
GCTATAAGTAGTTCACATTGGATTAGTATCAGATATAATTAGGATTAGGATAAATTAAGATTTATATAAAATATTTAAAATAATGAA
GAGTAATATTGACCATGGAAAGCAACATCATGACCTCATCTCCTTAAACTTGCGTTTCGCTTTTAAAACACTGCTTATACTTTAGT
GAAAAAGAAGTCACAATCTTCTTGGTCTGCGCCTAAATAGTTATCTCATTTCCAATTCTCAAAGTCCTATCAACGATAGTATCACA
AACTTATTCTGCAACAAGTATCTCTTGTTCTTCAGCTTTACATAAGTAATTCGAGTGTTCTGAAGATTATAGATATATTTGGCCCCA
TACATGGAGAGGTACAAAGGTAGGTCTCATAACAGACTGCCTCAGACACTAGAGTAGACACTAGGGTCTCTTGACAATAGAGCC
TCACACATGGAGACATAAAGTAGAAGTTCATATAAGGAGACCGATAGTAACGACTCGGACATTGATACAAACAATAGGGTCTTG
GATTTGGCTTTTTTGTTTTGAATCTTTACGGTCGTTGTCAGAGAGTGTTATATGAGGAGAAGATGAAGCTACCAGGGGGAAGCTT
AGAGATGGTACTTGAGCCGACAACCCTTGTGGCTGCTTTGAGTTTTAGGCATTATAAAAGAGGTATTATCATGGTTTTGAATATC
CCTAATATATCATTTTATAAGGCTTGCTGCTAAATGATATGATCTCAATTCATTTTCGGTTTTGTCAATTTTTAGGCGATATTATAT
CATGATTTTCCAATATATCAAGTTACATTTCAGCCTCAAAAATCACCAGAAATGTTGCAGTATCAATTTGGATTTTTATTTGTTATA
AAAGATTGGTCTGGATTTATGGTTCAATTGAGGCCTAAACCCGAACAAATATCTGTTAAATAACCAGAAACAAGTGGAGCTCGAT
TGAGACCGAATTCTCTTGCAACATGTTCCCAATATCTGATAGTTAGAACATTGGGTATTATAGAATTATCATTTTAGCTTCGGTAC
ATTGTTATGTTGCAGAAAGTAATTTCACCATAGTGAGGGTCTGAGGGATATTCAGAGTTTATTTATTGTAAATCAAAAACAACACA
CAAGTACATTTTTTCAATGAGAAACAACCGAAGTTCTGGAACTTTCCTAAATATATAACTGCTCTAATAATGGGTATTTGTTAAAG
TTGAGAAACGGTGAGAGGATGAGATGGAGACAGGAGCTATGCTGGATGAAACTGCTAGAAGCCTAGAACAAACTAGGTCTGAA
GCTATAATGGAGTAGAATGTGAATGGAGCCACATACAAATACAATAAAAAATATGGACTTAATAGGCAATAGAGAAAGCTTTCCA
TTTAGTAGATTCTAATCATGCAGGGTAAAGGCTTTGTTGTTTCAATTTCTGGTTTGTTGTTTTCTTGTTTCCCTCTTGGTGTCTGTG
TAGTTATAATGGAAATTCTGATAACAAAAACACCATTTCGTTTGAAAATGGATGTCAAAATCCCAAGAGCGGAAAAATTATTAGTC
CTAAAGCTATTAGCCTTCATAACAAGTAATGGAGGCTAAGCATGCATGCATGCCACCTTGTATAAATCATGTGAATATGCTTAATT
CCGTGTTCTAGAGGATTGCTTCTGCTGTTTTTTATATATGACAATTTGTTGTTTGCTATTCAAGGCATTTTCATTAGTTAGCAATTG
GGCATCAGAGTGGACATTCATATGCTTCATGCATCTTAATCTAACCTTTTTACAGGCACAAATGCTCCGCATGTTTCAAGCAATA
CAAGATAAAAGATCACCTTGTCGAACATATGAAGAAATCATATCATTCAGTTCATCAGCCCAAGTGTGGGGTCTGTAAAAAGCAT
TGCAAGACACTGGAATCAGTTAGAGAGCATCTTACAGGTAGCTACTTAAGCTAAACTTTAAGCATATGCAGTTATGTTTCAAATG
TTAGTTTTAATCTCTTACTATTTCTTATTTTTCACTCTATTAAATTTCAATTGCATGAAGGTCCACTGGCCAAATCTGACTGTGCAG
TGGTCTTTGCTGCCTTGGGTTGTAAGCTCTGTCTATCAATATTTGATAATGTTGATGCTCGTGGCCTACATGAAGTCTCTTGTAG
CTTTGATCCACCTTCTGTCCCTGTAAGTTATGCAACAAAAGCTTGATACTCATATATTCTTCTACTTAGTCTCTCCCATCTAAAGTT
GAATGGGTATTTTTATAGGAGCTAATGTCCTTGCCTATTGCTGACATGTGCGATGAAGTTGAAGCGGCAGTTTTGAACTCAGCTG
TGCCAGATGATAATGGTACTTGCCCCAAATTAGTCGCTTTGGACTGTGAAATGGTTGGAGGTGGAGATTATGGTTCATTTAACCT
TTGTGGTAGAGTTTGCCTTGTTAATGAAAAGGAGGAGGTGATATTTCATGCATATGTAAAGCCTATAATTCCAATCACTGATTACA
GGTTCTCTCTTTCAGTTCATTCCTGATGATTGTTATAGGAATGGGTTATTTAGATATTAGTCCATAAAGTCATCAAGAATTCCATAT
TAGCCTTCAAATATGTGAAATTGCTCGTGAGTGGGTCTTGCACTTGGTCGCTCAATGACCAAACTACCCTCCAGTCTATGTTCCC
TCTAAAAACCCTTCTCGTCCAGAAAAACATTGGCTAACGGGTATTTTGGTCATTGAGTGACCAAATTGCAAGACATCTTAGGATT
TGAGAGACATGCATAATTTCAAATATGTGAAGGATTAATATAGAACTATTGAAGATTTGAGAGACTGTATATAATAACCCTTTAGG
AATTTCTTGTGTTTGTGTATTCAGATTAACCTTAATTTGCAACTGGCTCCAGATATGAATTAACTGGCATAACAGAGGATCATATC
GTAGATGCAACACCAATTTGTGAAGTTTCAAAGAGAGTCAAAGAAATATTGTATAATGGGGAAGAATCGACATGGAGAATGCGA
ACTGATGGTGGAGAAGCTTGTCTTCTTATAGGTCATGACTTGGACCATGATCTTGAAGTTTTACGAATGGACTACCCTGAGAACT
TGACACGGTGAGCTCTTGATGATCCTCTTGTCCTCTTCTTAGTTGAATGCAAAATTAAATTGCATTTACAACCTTATGAAAAATTA
AAGTTTTTTTTTCTTTTTCTTTTGGGCTAACTCACTGAATTGTTCCTTTGATCTTTATACAGGGATACAGCCAAATACCGTCCGCTT
CTGAAAACAAATTTGAAAAGCCATTCTCTGAAGTATCTGACCAAGACCTACTTGGGGTGTGTAACTCGTTCGTATGTTACACTCC
CTATCAACACTGTAATATTTATAGTAAAATACAACTTCGTGTTCTTTTAGGATGTTTTATTGCTAAATTTAGTGGTTTAGTATCAAG
TTTCCTGATATAATTGTAAAGTTCATATTTGTTTAAGTGACATTGATTAAAGAATAATTCAACCATGTACTCAAACACTAAGTGTCA
AAGCGCATGACTTATACTCTTTAAGCTTGTAGAATTATAACATACGGACAAGTAGAAATTTTTTTTATCTGGAAATATGAAGTATCT
CGACTCTAATATCATCCTCTGTGGTTGATAATATGATATCTCTTGTTTACCCCGTATTACTAGGAAATAGTAAACAATTGAATTGAT
TGAGAATGTAACCACTGTTTTTGAATCCTAATGCATGCATTCATGTCGATACACTTCTTCTGTTAACAACTAGATAATCCTGTTTG
AAAATCCCAAAATGAATTTATTAACCAAGTTTTGGCTTTATAGGTATGAGATACAGTCTGGTACACATAATCCATATGAGGATGCA
GTTTCTGCTATGAGACTTTATAAAAGGATTCGCTCTATTGTGCACCAAACCAATGAAATTTCCATTCAAGATAATGATGATAGCAT
CTATTATACCAACGACAGCAAGAAGAACAGCCCATTTGATTCCAACAAGAACAATAAGAAGAAAAGCCCATTTGATCTATACAAG
AAATGTGAACTCTTGGCTAAATCCCCGGATGAGCTACTTGAGATATCCAAGTCCTCCTACAAATGTTGGTGCTGGGATGCAAAA
AGAACGCTAAACAGGATGATAGAAGAGTGA
SEQIDNO: 301
MGFLGPLPKAECEKIFKERGCDICLTFLASRNALRAHKESCQFSRSNNGLLYRMTRLGLGFQDDLKIGNSQGRVVALSCKMVGGGTD
GSLDLCARVCLIDEHERILFESYVAPNIPITNYRYEISGIRPEYMRNAMPLKQVSRKIQDYLCNGEPIWQIRTRSGRARILVGHNLDHDL
KCLEIEYPTIMIRDTAKYPPLMKTSKLSNSLKYLSKAYLGYEIQIGVQDPYDDCVATMRLYMRMKSQVHKRENYPLATDPQNRNNFAS
WRENELEKMMPPQLLEISRSDYYCWCLDS
SEQIDNO: 302
ATGGGATTTTTAGGACCATTGCCAAAGGCTGAGTGTGAGAAGATTTTCAAGGAACGAGGATGTGATATTTGTTTGACATTCCTTG
CTAGCCGGAATGCACTAAGGGCTCACAAGGAATCATGTCAATTCTCACGCTCAAATAATGGTTTGCTCTATCGCATGACTAGGT
TGGGATTAGGATTTCAAGATGACCTAAAAATTGGCAATAGTCAAGGAAGAGTTGTTGCTCTTTCCTGCAAAATGGTGGGAGGTG
GCACCGATGGCTCTCTTGATCTTTGTGCAAGAGTTTGCCTCATTGATGAACATGAAAGAATCCTCTTTGAATCCTATGTTGCACC
AAATATTCCCATCACTAACTATAGGTATGAAATATCGGGGATAAGGCCAGAATACATGAGAAATGCAATGCCACTAAAGCAAGTA
TCAAGAAAAATTCAAGATTATCTTTGTAACGGGGAACCTATTTGGCAAATTCGTACTAGAAGTGGAAGGGCAAGGATCCTTGTTG
GTCACAATTTGGATCATGATTTAAAATGCTTAGAAATTGAGTACCCAACAATAATGATAAGGGATACGGCAAAATACCCTCCACT
AATGAAAACAAGCAAGCTGAGCAACTCACTCAAGTACTTGAGCAAAGCTTATCTTGGATATGAAATTCAGATAGGAGTGCAAGAT
CCATATGATGATTGTGTTGCAACAATGAGGTTGTACATGAGAATGAAATCACAAGTTCATAAAAGGGAGAATTATCCACTTGCAA
CTGACCCACAAAATCGTAATAATTTTGCATCATGGAGGGAAAATGAGCTTGAGAAGATGATGCCTCCACAATTACTAGAAATCTC
ACGATCTGATTATTATTGTTGGTGTTTGGATTCCTAG
SEQIDNO: 303
ATGGGATTTTTAGGACCATTGCCAAAGGCTGAGTGTGAGAAGATTTTCAAGGAACGAGGATGTGATATTTGTTTGACATTCCTTG
CTAGCCGGAATGCACTAAGGGCTCACAAGGAATCATGTCAATTCTCACGCTCAAATAATGTATGCTTATTCCAACAACACAACTA
TGGTTAAACGTAACTCAACTGACACCTTATTTTTTCTCATAGTTTTAAATATATTTGTTAATTTTCAGGGTTTGCTCTATCGCATGA
CTAGGTTGGGATTAGGATTTCAAGATGACCTAAAAATTGGCAATAGTCAAGGAAGAGTTGTTGCTCTTTCCTGCAAAATGGTGG
GAGGTGGCACCGATGGCTCTCTTGATCTTTGTGCAAGAGTTTGCCTCATTGATGAACATGAAAGAATCCTCTTTGAATCCTATGT
TGCACCAAATATTCCCATCACTAACTATAGGTAATTAATTAATTAATTATCGTACTAAAGTTTAATTAATTAACATAATATAGACGAT
TTGATCAATAATATTGTTAATGTTTAGGTATGAAATATCGGGGATAAGGCCAGAATACATGAGAAATGCAATGCCACTAAAGCAA
GTATCAAGAAAAATTCAAGATTATCTTTGTAACGGGGAACCTATTTGGCAAATTCGTACTAGAAGTGGAAGGGCAAGGATCCTTG
TTGGTCACAATTTGGATCATGATTTAAAATGCTTAGAAATTGAGTACCCAACAATAATGATAAGGTAAACACAACATGCATGTACA
ATTAGTAATATGATAAGAGAATAAAATTTACTAAATTACATTAATTACTCCTATTAATTACAATCAAAGTAAAAATTATATGTAAATT
TCTCCTATTGTTTAATGTTTTTTGTTTTTAATATTTTTAATTTGTATGTAGGGATACGGCAAAATACCCTCCACTAATGAAAACAAG
CAAGCTGAGCAACTCACTCAAGTACTTGAGCAAAGCTTATCTTGGGTACGTAAATACTTTAAATTGTTAATTATTGTGACTTTTTA
TGTTCAAATTCACACATCGTCCTAATCAGAATCAATCTTAATTGTCTTGTAATCGAATAGATATGAAATTCAGATAGGAGTGCAAG
ATCCATATGATGATTGTGTTGCAACAATGAGGTTGTACATGAGAATGAAATCACAAGTTCATAAAAGGGAGAATTATCCACTTGC
AACTGACCCACAAAATCGTAATAATTTTGCATCATGGAGGGAAAATGAGCTTGAGAAGATGATGCCTCCACAATTACTAGAAATC
TCACGATCTGATTATTATTGTTGGTGTTTGGATTCCTAG
SEQIDNO: 304
MANLGIQDELRIDNNRGRVVALACKMVGGGSDGSLDLCARVCLIDEHERILFHSYIKPNIPVTNYRYEMTGIRPEYLRDAMPLRNVSR
KIQEFLCNGEPIWQIRSKGGRSRILVGHGLDHDLKCLEMDYPAIKMRDTAKYPPLMKTSKLSNSLKYLTKAYLGYDIQIGVQDPYEDCV
ATMKLYMRMKLSQFHKKENYPLATDPQNKNNFASWRQNELERMTPDQLLDFSRSDYYCWCLDSQDY
SEQIDNO: 305
ATGGCTAATTTGGGGATTCAAGATGAACTAAGAATTGATAACAACAGAGGAAGAGTGGTTGCCCTTGCTTGTAAAATGGTTGGT
GGTGGAAGTGATGGATCTTTAGATCTTTGTGCAAGGGTTTGTCTCATTGATGAACATGAAAGGATCCTCTTTCATTCATATATCAA
ACCAAATATTCCTGTCACTAACTACAGGTATGAAATGACGGGTATAAGACCAGAATATTTGAGGGATGCGATGCCTTTAAGGAAT
GTGTCAAGAAAAATTCAAGAATTTCTTTGCAATGGGGAACCTATTTGGCAAATCCGTTCTAAAGGTGGAAGGTCTAGGATTCTTG
TTGGCCATGGTTTGGATCATGATCTTAAATGTTTGGAGATGGATTACCCAGCAATAAAGATGAGGGATACTGCAAAATACCCACC
ACTTATGAAAACAAGCAAGCTCAGCAACTCCCTCAAGTACTTGACCAAAGCTTACCTCGGGTATGATATTCAAATTGGAGTACAA
GATCCCTATGAAGACTGTGTGGCCACAATGAAACTCTACATGAGAATGAAATTATCACAATTTCACAAAAAAGAGAATTATCCTC
TTGCTACTGATCCACAAAATAAAAATAACTTTGCATCTTGGAGGCAAAATGAGCTTGAGAGGATGACTCCTGACCAACTGTTGGA
TTTTTCAAGGTCTGATTATTATTGTTGGTGTCTGGACTCACAAGATTATTAA
SEQIDNO: 306
ATGGCTAATTTGGGGATTCAAGATGAACTAAGAATTGATAACAACAGAGGAAGAGTGGTTGCCCTTGCTTGTAAAATGGTTGGT
GGTGGAAGTGATGGATCTTTAGATCTTTGTGCAAGGGTTTGTCTCATTGATGAACATGAAAGGATCCTCTTTCATTCATATATCAA
ACCAAATATTCCTGTCACTAACTACAGGTGATCAATTTAAATTAATAACGTAAAAAAATCTTAACACAATAAATAAATTTCCATCTT
TATTATAACATTTTATTTAGTTACTATGCTATCGAACGGAGTAATAATACTTAGCTTGTTGTGTAAATATTTTCACATTCCTAGTACA
TAAAATTTAAACTCAAATATTAAAGCATGTTCACTTGTTTTTGGTCTTTTTGGGGCCCATTGTAAATTCCTACGACACCATGACAA
GAAGGTATAAGAATTTTCTTATATTGACTATGTATATTAACTTAAACTCTGACGAGAATTATATATGTTTATAGGTATGAAATGACG
GGTATAAGACCAGAATATTTGAGGGATGCGATGCCTTTAAGGAATGTGTCAAGAAAAATTCAAGAATTTCTTTGCAATGGGGAAC
CTATTTGGCAAATCCGTTCTAAAGGTGGAAGGTCTAGGATTCTTGTTGGCCATGGTTTGGATCATGATCTTAAATGTTTGGAGAT
GGATTACCCAGCAATAAAGATGAGGTAAATTAATTATTTGATCATTTATATAATCTAAAAATAATTTCATAACTTTATTGCTATAAAA
AACATGAAATTGACTCGTACTCATAGTTGTTGTTATTATATAGTAGTATATATTACTCCGATCCCTATAGATGAACGTTCTAACAAA
GACAAACAACACGGGAAAATTACAGTCAAATGTCTTTAGGTCATCTAGTTTACAAAGTATATACACAGTAGTAGTGTATATTTTAT
ACTAAATATATATACATATATTTATTCATTTGATATACCTACCTTATACATATAAGCAATATATATAGCTTGAAGTATATAAATAGTG
TATATTTCGACCATATTTACGTGACTTCCCTTCTAACATTTGTTTAATTTTTTCTTTTTTCTGAAGGGATACTGCAAAATACCCACC
ACTTATGAAAACAAGCAAGCTCAGCAACTCCCTCAAGTACTTGACCAAAGCTTACCTCGGGTAATATATATTAATAGTGCATCAA
ACTTTATCCTAATAAATTTTCTTGAGTACCACAACTATATATTATTAATTTAACTTGTTGCAAGTTACGTTTTATAAAAAATCTAAAA
ATTGATGGACAATAATATAATGAAAAATGTGTAGGTATGATATTCAAATTGGAGTACAAGATCCCTATGAAGACTGTGTGGCCAC
AATGAAACTCTACATGAGAATGAAATTATCACAATTTCACAAAAAAGAGAATTATCCTCTTGCTACTGATCCACAAAATAAAAATA
ACTTTGCATCTTGGAGGCAAAATGAGCTTGAGAGGATGACTCCTGACCAACTGTTGGATTTTTCAAGGTCTGATTATTATTGTTG
GTGTCTGGACTCACAAGATTATTAA
SEQIDNO: 307
MDYQASETLRNKCAACFRQFNKMEHLVEHMRISYHSVHEPMCGICKKHCRSFESLREHLIGPLPKQECKNIFSVRGCKICLAIFDTPN
ARWVHQERCQLSNANTGLVGRFANLGLRDNLTIDCASRGPQVVALACKMVGGGSDGSLDLCARVCITDEYNNIIFHSYVKPPIPVTN
YRYEITGIRAEYLRDAMSLRQVKKKIQDFLCNGEPMWKIRPKAGRARILVGHGLDHDLDRLQIEYPSLMIRDTAKYPPLMKTSKLSNSL
KYLTQAYLGYEIQNGIQEPYEECVATMRLYMRMRNQSHKKEDYPLASDPQNRNNFASWRQNELERMIPEEMLAISRSDYYCWCLD
SEQIDNO: 308
ATGGATTACCAGGCCTCTGAAACCCTAAGGAACAAATGTGCAGCCTGTTTCAGGCAATTCAACAAGATGGAGCACCTGGTGGA
GCACATGAGGATTTCTTATCACTCGGTGCATGAGCCCATGTGTGGGATCTGTAAGAAACATTGCAGATCTTTTGAGTCTCTAAG
GGAACATCTTATAGGGCCCTTGCCTAAGCAAGAATGCAAGAACATATTCAGCGTCCGAGGATGCAAAATCTGTTTAGCGATCTT
TGACACTCCTAATGCTCGTTGGGTTCATCAAGAAAGATGCCAGCTTTCTAATGCAAACACTGGGTTAGTGGGTCGATTTGCAAA
CTTAGGGCTGCGTGACAACTTGACTATTGATTGTGCGAGCCGAGGTCCTCAAGTAGTTGCATTGGCTTGCAAGATGGTTGGGG
GAGGGAGTGATGGGTCCCTAGACCTCTGTGCCAGGGTCTGCATCACCGACGAATATAATAATATCATCTTTCATTCTTATGTCAA
GCCACCGATTCCAGTCACAAATTACAGGTATGAGATAACGGGAATTCGAGCTGAATACCTGAGAGATGCAATGTCACTAAGGCA
AGTGAAGAAGAAGATTCAAGATTTTCTGTGCAACGGAGAGCCAATGTGGAAAATTAGACCCAAAGCAGGAAGAGCAAGGATTTT
AGTGGGTCATGGTTTGGATCATGATCTTGATCGTCTACAAATTGAGTATCCTTCTCTCATGATCAGGGATACTGCAAAATACCCG
CCATTGATGAAGACAAGCAAGCTCAGCAACTCACTCAAGTACTTAACTCAAGCATACCTGGGGTATGAGATACAAAATGGGATA
CAAGAGCCATATGAAGAATGTGTTGCAACAATGAGGCTTTATATGAGAATGAGAAATCAATCTCACAAGAAAGAAGATTACCCTC
TTGCTTCTGATCCTCAAAACCGCAATAACTTTGCTTCGTGGAGGCAGAATGAGTTGGAGAGGATGATACCAGAAGAGATGCTGG
CTATTTCAAGGTCCGATTACTACTGTTGGTGCCTCGATTGA
SEQIDNO: 309
ATGGATTACCAGGCCTCTGAAACCCTAAGGTATATACGTATATGCATACACGTACTACTCTTACTAGCTAATTTATACACCACATC
TGGAAGTAATCCTTTATTAACTAACTAATATATATATATATATGTATATGTATATGCATCATGTAGGAACAAATGTGCAGCCTGTTT
CAGGCAATTCAACAAGATGGAGCACCTGGTGGAGCACATGAGGATTTCTTATCACTCGGTGCATGAGCCCATGTGTGGGATCT
GTAAGAAACATTGCAGATCTTTTGAGTCTCTAAGGGAACATCTTATAGGTATGTAAAGCATTTATATCTATATATATCTGTATACA
CATTTTCTAAAATTCATGGTGATATGATATAATTTGTTTTCATTTAAATGTATATATATATTAGGGCCCTTGCCTAAGCAAGAATGC
AAGAACATATTCAGCGTCCGAGGATGCAAAATCTGTTTAGCGATCTTTGACACTCCTAATGCTCGTTGGGTTCATCAAGAAAGAT
GCCAGCTTTCTAATGCAAACACTGTAATTATTATATTTATATATACATATTAATATTCATATATATTCTGCATATTTTGTTGTAATATT
TTTTATTGAATTTCTACTTTGAACATACATATATATATATATATAGGGGTTAGTGGGTCGATTTGCAAACTTAGGGCTGCGTGACA
ACTTGACTATTGATTGTGCGAGCCGAGGTCCTCAAGTAGTTGCATTGGCTTGCAAGATGGTTGGGGGAGGGAGTGATGGGTCC
CTAGACCTCTGTGCCAGGGTCTGCATCACCGACGAATATAATAATATCATCTTTCATTCTTATGTCAAGCCACCGATTCCAGTCA
CAAATTACAGGTATACACACACGTACACACACACACACTATATATCATAATGTATTTGGAGACATCCCTTAATATCATCTTTGTGA
TGCTACCAAGTGAAATTAATTAATCGGAAGAGTTATCCAACATATATAAATAGTGTAAATTTTTATATTTAATTAGCATGAAATTGT
ATATTTATGCATTTTTATATTAATCTTATGGTAATGACACCTAATGATATAATAATTATTAATTAAATTTTAATTATATATGATTAAAT
TTAAGGTATGAGATAACGGGAATTCGAGCTGAATACCTGAGAGATGCAATGTCACTAAGGCAAGTGAAGAAGAAGATTCAAGAT
TTTCTGTGCAACGGAGAGCCAATGTGGAAAATTAGACCCAAAGCAGGAAGAGCAAGGATTTTAGTGGGTCATGGTTTGGATCAT
GATCTTGATCGTCTACAAATTGAGTATCCTTCTCTCATGATCAGGTATATGTATATATATGTTTACACACACAGAAAAGAGAATTA
ATTATATTATATTTTCTCTATTTGTCTCGACAAAGTCTTTGAGATCTATTAAGCAACACTTTATGTATATACATATAAATATGGATGG
AAATTTATTTGTGAATTAATATAGGGATACTGCAAAATACCCGCCATTGATGAAGACAAGCAAGCTCAGCAACTCACTCAAGTAC
TTAACTCAAGCATACCTGGGGTGAGAATTTATAAGATGTTTAATTAATTAAATTAATATTTATTTATTTATTCATGAAGTAAAGAATC
TGTAATATATGTACGTGTGTATATATATAGGTATGAGATACAAAATGGGATACAAGAGCCATATGAAGAATGTGTTGCAACAATG
AGGCTTTATATGAGAATGAGAAATCAATCTCACAAGAAAGAAGATTACCCTCTTGCTTCTGATCCTCAAAACCGCAATAACTTTG
CTTCGTGGAGGCAGAATGAGTTGGAGAGGATGATACCAGAAGAGATGCTGGCTATTTCAAGGTCCGATTACTACTGTTGGTGC
CTCGATTGA
SEQIDNO: 310
MTHARVNHHNPGHNHRVNHHHLHQTATTTVKVDEKWKVIEVARRKMNRERHRRLFSKNKCAACYRQFNRKEHLVDHMRTSFHSV
HEPMCGVCKKRCRSFESLREHLIGPLPKVECAKIFSSRGCNICLTILDSPYTLRMHRGACQLSRPNALISSMANLKMCDNMKIDYGRE
RSPNVVALACKMVGGGNDGTLDICARVCLTDEYDNILFQTYVKPHIPVTNYRFETTGIRPDYLRDAMPLKQVQRKIQDFLCNGEPVWK
IRSRGGKARVLVGHGLDHDLECLGMEYPALMLSYEIQIGVQDPYEDCVATMKLYMRMRSQNHKAEYYPLASDPQNRNNFAQWREA
ELEKMTPDELLAISRSDYYCWCLDLKGYM
SEQIDNO: 311
ATGACCCATGCCCGCGTCAACCACCACAACCCAGGCCACAACCATCGTGTAAACCACCACCATCTACACCAGACTGCCACCAC
TACCGTGAAAGTCGACGAGAAATGGAAGGTGATCGAGGTGGCTCGTCGAAAAATGAACCGGGAACGCCACCGTCGGCTTTTCA
GCAAAAACAAATGTGCTGCATGCTATAGACAATTTAACAGAAAGGAACATTTGGTGGATCACATGAGAACCTCCTTTCACTCTGT
TCATGAACCTATGTGTGGTGTTTGTAAAAAGCGTTGCCGATCCTTCGAATCATTAAGAGAACATCTTATTGGACCATTACCAAAG
GTGGAATGCGCAAAGATATTTAGTAGTCGAGGATGCAACATATGCTTGACCATTTTAGATAGCCCTTACACCCTCAGAATGCATC
GAGGAGCATGTCAACTCTCTCGACCAAATGCACTAATTTCTAGCATGGCAAACTTGAAAATGTGTGACAATATGAAGATCGATTA
TGGTCGAGAAAGAAGTCCTAACGTAGTTGCGCTTGCATGCAAAATGGTAGGAGGTGGTAATGACGGAACACTTGATATTTGTGC
AAGGGTTTGCCTAACTGATGAATATGATAACATTTTGTTTCAAACATATGTCAAGCCTCATATTCCAGTGACTAACTATAGATTTG
AAACCACGGGTATCAGACCGGATTACTTGAGGGATGCAATGCCATTGAAGCAGGTTCAAAGAAAAATTCAAGATTTCTTATGCA
ATGGAGAGCCGGTGTGGAAAATTCGATCAAGAGGAGGAAAGGCTAGGGTTCTTGTTGGCCATGGACTAGATCATGACCTTGAG
TGCCTAGGAATGGAATATCCCGCATTGATGCTTAGTTACGAAATCCAGATTGGTGTCCAAGATCCTTATGAAGATTGTGTTGCTA
CAATGAAACTCTACATGAGAATGAGGTCTCAAAATCATAAGGCAGAATATTATCCATTAGCCAGTGACCCACAAAATCGCAACAA
CTTTGCTCAATGGAGGGAAGCTGAACTTGAGAAAATGACTCCAGATGAACTATTAGCAATATCAAGGTCCGATTACTATTGCTGG
TGCTTAGACTTGAAGGGATATATGTGA
SEQIDNO: 312
ATGACCCATGCCCGCGTCAACCACCACAACCCAGGCCACAACCATCGTGTAAACCACCACCATCTACACCAGACTGCCACCAC
TACCGTGAAAGTCGACGAGAAATGGAAGGTGATCGAGGTGGCTCGTCGAAAAATGAACCGGGAACGCCACCGTCGGCTTTTCA
GCAAGTATGACTTTTTTTTGGGGGGTTTAAAGTGGTTAGATTAGCCGGAGAAACTGCCGTTTGACGACAGTAAACCACAGCCAC
AAACTGGTTAACCGTCACGGTAACCGGATTTGGGCTTTGATTACCGTGGTCAACCTGAGCTGAAAATCGGTCACCGTCCACGAT
AACTGGTTTAGGGCTCCGGTTACCGTGGTCAACCGGAGCTAAAAAATCGTCACGGTAACCGGTTTTGACTTACGGTATTAGCCA
TCGATTTTATTAAAAAAAACGAAAAACAGAAGAAAAAAAAATCCACGAGAATACTTACCTCCTTTTGAAATTTACATGGTACAGCA
GGGTGTACCATGCTCATGGTACACTTGGAAGAAAATTGTCATTAAAAAAAAAAAGTTAGCTTCCTAAATTTATGCTCCTTAATTGA
AGGATTTTTGTTATTACCTTTCCATAATTTTAGCTTTTCATATCCATAATTGTTCATTCAGTTTTTGAAAAAATAGTAAATTTTAGAG
TAGAAATAAAATTCCAAGAGAAAAAAATAAATTTTTTAGAGAGAAAATGATGGCTCATGATGAATTTTCACGATATATGATTGTTG
ATTTGCACTTGATGATCACCAAGTTGATGTTTTTAACAAATGATATTTAATTTTAAATTTTGTCACCAAATGAGAAGGTACTTTTTA
ATTAATTTATTTTTTATTTATATTTCATAATTTTGTTGTTGATGATGATGTTTTTAGATTATTATATTTGTTCATGGATCATTGTTAAC
GAATTCATCACGGCAAGAAAATGTAATTTTTTTTTTTTTTGGTTATTTAGTTTTTTTTTTTTTAATTTGTGTTATTTTCGAATGTTGAA
AATGAAGTTCATTTGAATTGGGATAAATGTTCTTTTATCAGATTTTTTGATCCATATTCATTTTTTTTAATTGGAATGAATACGTTCA
TTGAGTTTAATTAATATATTTTTTGGGTCTAAACTATACGTTTATTGAGTTTAATTAATATGTTCTTTAATGATCACCAGATTGATGA
TAATTAATTTTAGATTTTATCACCAAATGAGAAGGTAGTTTTTAATTATTTTTTATTCAATTTATATTTCATAATTTTGTTGTTGATGA
TGATGTTTTTAGATTATTATATTTGTTCATGGATCATTGTTAACGAATTCATCACCGAAAGAAAATGTAATTTTTTTTTTTAATTATTT
TAGTGTTTTTTTTTTAATTTGTGTTATTTTCGGATGTTGAAGATGAAGTTCTTTTGGATTGGGATAAATGTTCTTTTATCAGTTTTTT
TGATCCATATTCATTTTTTTTTAATTAGAATGAATACGTTCATTGAGTTTAATTAATATGTTATTTGGGTCTAAACTATATGTTCTTC
ATAGTCACAAACTATGTTCTTTGGATTTGAACTATATGTTCTTTACGTTACAAACTATGTTCATCTATTTGTCTATTTTTTTCAAAAA
AATATTATCTAATTGTGTATATGTATATTTTTAATCATCTAATTGTGTATATGTATAATTTTGTTTTATAATAGATCAGGATCATAAAG
GTTGATGAAGACGATATACTTTTGGGACCTCTTCTATTGATGATTGATTTTCCGATAGATTTAAAGAACATTCGCTATATTGATAA
AGAACATGATAGTATTATTTAAATAACATTATTTATATTTAACCAACATGTTAAAATAAAATTCAGGGAACATTTGACAATAAGTAT
GAACATTGTGTGACAATAAAGGTACACTAAAATTGCAATCAAAGAACATGGACATTTAAAATCATATAATGAACATCATAATAAAT
ACACATGACCAAAATGAATATTATAACAAAATATAAAAATATATAATTGGTTATTTACAGAATTTTTTTTAAAAAAAGAAAGAGAAC
AGGATTAAAAAAAAATATATTAGAATTTGAAATTTTGAAAATAAAATCTTCATAAAAAATAATTTTAGAGACAAAAATTATAGAAAAC
TGAATATTTTGAAATGATTTGACATTAAATACGTGGTAAAAATCAATTGGTGATTATCTTTTACCTTTTTTTTAAATTGATTCACCAA
AATTACCACGTTCTTTATATTTCAATGTTGTGTTCTTTTATAAAGTCGTGATGTTTATTACAAATATAAGTTATGTTCTTTCCGCTTT
TTATTTTAACTAGTTTTGATCCGGATTTACCATGCGCACGGTACACCCGAGTATACCAACCAACGATTACCTCCTTTCCAGCCAT
TCCGCCACCTGAACCGCCGCCTCTTGACATCTGCCTGACCAGCCGCCTGCTTTCGCGTCGCCGCCAGCAACTTCTGCCGGTAA
CCTCTACTATGTGGTTGTGGTGGTTGTGTGGAACAAGGTTGGTGGTTGGGGTGTTAGTTTGATGAGAGAGAAAATAAAAATAAA
AAAAAATAAAAATAAAAAAGGGGGAGAAAAAATGGGTTAGGGTTTAGTAAGGGTAATGATGACCCTTTGCTTGAAATGATGGCG
GTTTTGATGAATTTGGATGGCGGTATTTAACACCCCTTTATAACTGCATGAAAATCTCATTTTCAGAAATTTACCATGAGATCCAC
TCTAGTGGATTATTACAGTTAATTTCCTTACATATAAATCATAAAATAACTATCAAAGCTAGTCGAAGGGGAGTATATGAATAGTG
TGAATGAAGGTTATAGTGTTGCAAGTAAAAAAATGAGGGAGCTGAGGTATTATTTTTCTTTCTTACTAATATTTAAATAAATTTCTA
AGCTACTAACTATTTTTTATAATAATATAATATTCTCACTGTGCACTGCCTATTATTGCAATTAATCAAAAACTAGGAATTGTCAGT
ATATAAAATTTTGTTAAATAACGAAAATTTTGATTTTTAACACCACAAAAAAATTTTAAAAATTCGATTTTACCACCACTTGACGGAT
TCTTGGATGAACCCGTTAGTGGGGCCTACAATTCCCACATGTCATCATATAATTGGTTAAAAAAATAAAAAATCAAAATCCTAAAA
CTAACCCAGCCACCCCACCCCCACTGCTGATCGCCCCTGCCCCACCTCCAACACCCCTTTTCACAACCCTAGCCACTAGAAAC
TCCGCCACCTCCCTTTCTCCTACCCCAAAATCGGCAGCTTCCCCCAAACGGCCACCAAAAACGCCGAAAGTGGTCGAAATTGA
CCGGTTTAGGGTCGATATCGACCACTCAGGCCCTCAACCGGCTGATTTCGACCGTTAGTGACTGATTTCGTGGCCATTTTTGGG
AAAGGGAGGGGAGGGTTCCGGTGGGGCGAGTTCAAGCTAGGGGAGGGGGTGCGTCGGAGGTAGGGCAAGGAACGTCGGCT
GCGTTGGGTAGGGGTGGAGAAAAGGGTGGCTGGATTAGTTTTAGGCTTTTGTGAGAAGGGTCACTAAAGAAATTAAGGATAAA
AAAAATGGATTATATAGTTTGACAATTGAAAGAGTCAAAGAGTACATTAGTACCAAACTTTCTTTAGATCTTTGTCTACCGACTTTA
ACTCCTCAAATGATAGAGAATTATATAGCTCAAAAAATGAATGAGGACATAAAAGCCGCTATATAATAAATACTTTTTTTGTATGT
CTTTCAAACGTAATCTTGCATATTCAAAGGGAATCTAGTCAAAATTTGACCATCCCTTTTAAAAAAAAAAAAAAAAAAAAATATATG
GGCTTTCGAAGGAATCAATAAGATCATAAAGTTAAGCAAATACATAGATTCCCTTCAATGCACTCCACATTCTTATAACCGTGTTT
AAAACCTTTAGTGCTATCCACATCAAAAAGGCTAACTTGTTAAGAGGTGAAGATAATATTACAATGAAAAAAACATGTAATTTCGC
AAATATTAATCATCGTACCCTCTAGTTAATCGTATATTAATTAGGAGATGATTATAAAATTTCAAGAAAAATTCAAAAATTAATAAT
GTTCTCTCGTGACATATATGAATAAATGGTAATTATATAATGATTAAAACCACAAGTTTATTTTGTGGTTCCAACAGCAAAGAAAT
CGCTTAGCTTATTGAGAACATTATATATATGCCAAATCCACTAGAGTGTTGATACGTATGCACGGGGCAGCTTTTGACCATCTAT
GGGAACCAAGAAAGCATCCTCTTTTTATTCATCATTGCATGTAAGCAATATTAACCTGTGTGGAATACTAGTACATGATAAATCTA
TAACGACAAAGCCTGTTCCAACATACAAGTCATATTCGAGACCAAAAGTTGTACAATGGGATCACCAACCAGCTCAGGTAGCAT
ATTGCAACAATAAAGATAACTCACGGTCAAAATAAGTTCTAGAGTAATCTAGCCATTGACACCATACTCTTAAGCACAATGAAATT
AAACCATACCTTATATTGGCGTGTAATGCTCCCTTCTCTTAGCTTATTGATTGTATAAGCCAAAAGGGACCATTATTAGTGGCCTA
TAATCTCTAAAGAAGCCTTGATTAGAGCTTGTAAAAGTGGGGACAACTTAAAAGCTAAGGTATCATCAGAAATTATAGAGAATATT
GTCAATAGAATGGAAGTCACAAAGCCATATATACGTGCATGTTAAAGCTATGGAACCATAGCATGGACCATATATTCATTTCTCT
TTGACTTTGGGTATCTTTCAAAGTTCAAAAAATTATCTCTTGAGGCCCACCTCAGTCAAATGGCATACTCTAGTCTAGGCTGACAT
CCTCCGAACATTTCACTGCCTCTAATAGCTTGAGTCATAAATGCCTCTAATAGCTTGAGTCATAAGGTTGGTATTAAAATGTCTTT
ATAAAATGACAAAGAATTTTGATGATATTTTGCAATTGTTGGTATTAATGAAGAAGTATTATATTTTGGTATCATGGCAATACCATC
TAACCACAATTTAATACCAACAAATATTATTTGAGTCATTCATGACTCAAGCTATTAGAAGCAGAGAATTTCTCGACATTCCCCAT
GCTCATTTTCAAAAGTACACAAATCCCTCCCTTCCTAGCTCTCTTTCCACGACGTACAAATGGTAGCACATATTGTAGCTGATAC
GCGGCAAATTAAAACCAATGAAAAAGAGATACGCTGACATAATATGGTAGTAGATTATAGATTCTTTTGTTTCTTAATTGTAGAAA
CTTGAAATATAAAATAATAACCATTACATGCAAATTATTTATTTGTCGAGTTGTCATTTTAGCCACGTATAACTGCTAGTATTACTT
TGATTAAGCTGCAAGATTGACAAAAAGGATAGTAAGGAATATAACCCTATTCGAATGCACTGATCATTTTATTGCATGCGAACAA
AAATCGGTCCATGATGACATTATCCTTGAGGAATCTCAGATTTTTATCTTCCTTTTTGCTGCCTTTCAACTGATATAAATTTTGAGC
TTTGTAACCCTCAAACCTCAATCTTTTCTTACATTGTTATTGTCTACGCTCACACATCAAGAGCGCGAGCATTATTGAACTCCCTT
CAACCGACAACAAACCATCATGGACTACAGATCAATTGAGTCTTCTGAATCCTCAAGGCATTTTTCTACTCTATACATTCTCTATC
ATAAAATGTTTGCAGAAGTTTTTTTTTTTTTTTTTTTTTTTGTTAAACTTGTTGCATATTGCCATATATCATGATTTTATGTTTCATGT
TTATGTAAATATTGATGCATAGTTGTATTATCAATGTCAAGTAGACGGGTATAAACGTTACCAAAATATATCATCCAATCAATATGA
TTTGATCAAATTAAACAAGTTTGCTCGACCTACTATTAGTAATCTAATTATGTATGTTOTTAGTACATGTAATTGTGAGTTCAATGA
AGTCCAAAGTAGGAATATCTAACGTACTTTTAGCTTTGGAACTTAATTAAAAAAACAATTTCAGGAAAAAAAGAATGAAATGTTTA
TAAAATTCCTTATTTTCTTTCTTAAGAAACCCTAGATATGATATGTTTCTCCTCTCTTGTTCGTTGTCCCTATGATTTTGAGTTCATC
TCAACTCTGCTCGCTATATAACTTGGAAGAAAATTAATCCAATGTCTTCTTTAGTCTTCACTATAATAAACTCATGGAAAGTAATCA
AGTATATTTCTTTTTCCCTTTCTTCTACTTTCTATTTTGGTCACTTGCAAATTTAAGTCTTCTATTGCCTATCCTTTTCTTGTGTTCT
AGTTTTACTAATAATTATAGACAGATTCATTATTTTAAAAGATAATGAACATCGATCATAATACTTTTACATATTATTATCTCTCAAG
CTAATTTCGAGAGTTCTTTAAAGTCAAGTTTTGTTTCAGTATTATGTTGACTGAATTGAAATTATCTCATCACATTTATGTTCTTGTT
TAAAAATATAATTTCATTTAGTAGCTTATCATCTCATCTTGAAATGTTATCTAACATTAAAGCTTTATGTAATGCAGAAACAAATGT
GCTGCATGCTATAGACAATTTAACAGAAAGGAACATTTGGTGGATCACATGAGAACCTCCTTTCACTCTGTTCATGAACCTATGT
GTGGTGTTTGTAAAAAGCGTTGCCGATCCTTCGAATCATTAAGAGAACATCTTATTGGTAGGTTTATATCCTATAATATTACTATC
TCCGTTTCATATTATGTGCAACATTTATTAAAAACAGTTGTATGCTATGCAACAAATGTTTTCTTTCTTTTTTAAGCTGTCACTTCTC
GCTATCTATCTCCTCATTATCACATGCATCATTATCTTTCTCTCTCCATTTAACACTCACGGACAAATTTTGCCTATCTATATTGTT
TTCACAGGACCATTACCAAAGGTGGAATGCGCAAAGATATTTAGTAGTCGAGGATGCAACATATGCTTGACCATTTTAGATAGCC
CTTACACCCTCAGAATGCATCGAGGAGCATGTCAACTCTCTCGACCAAATGTAATGACTTTAGCTTGGAAAAACAAATGTAACAA
ATTAATAGTATTTCCATCAAAACTTTGTAAATTGAACCTTTTTTTTTTTTTTTTTATGCAGGCACTAATTTCTAGCATGGCAAACTTG
AAAATGTGTGACAATATGAAGATCGATTATGGTCGAGAAAGAAGTCCTAACGTAGTTGCGCTTGCATGCAAAATGGTAGGAGGT
GGTAATGACGGAACACTTGATATTTGTGCAAGGGTTTGCCTAACTGATGAATATGATAACATTTTGTTTCAAACATATGTCAAGC
CTCATATTCCAGTGACTAACTATAGGTAACTTTTATTTTCGACATTAGTTTCTCTACTAGCTTGATTTAAATTATOTTATACACGAC
TCATTCACTTATTGTATTTCGTGTGTAGATTTGAAACCACGGGTATCAGACCGGATTACTTGAGGGATGCAATGCCATTGAAGCA
GGTTCAAAGAAAAATTCAAGATTTCTTATGCAATGGAGAGCCGGTGTGGAAAATTCGATCAAGAGGAGGAAAGGCTAGGGTTCT
TGTTGGCCATGGACTAGATCATGACCTTGAGTGCCTAGGAATGGAATATCCCGCATTGATGCTTAGGTATGAACTATTTCCTTCG
TTTCTTAATATTTGCATCAATTAAATATACACGTTTTTCAATGCATCAATTATATTTAGATAATAATACATATTGAGATTAACTCACC
ATTAAAATAATAAAATTTATTTATGCTTCGTGTACATTATTAGTTGTAAAATATGGTCAAAGTTAGTATAATAAGAGTGTATAAGTTA
ATTGATACAAATATTAAGAAACAAAGGAAGTATATTGACAATTATTAATCACGTGCATATAAATGAGTTCATAACTAATATAAGTCA
TTTGATGGTTTGATTTAATTTGATTATATATAAGTGCTCAATATCTCTTTCTAATCAAACTTTGATGATATAAATTTGTTTGTTGGGA
ATTGGTAGAATTATATTGAGAAAAGTCAAGGTTGTTACACAAGGGAAATGATAAAAAAAGAATATATAGAGCACAAACAATTTGTA
GTTGTGAGGGTCCTTAGGGTTTTAAATATCTTGCAAAGAAACTTAATTGTAGATTCGTTCTATCTAGCAAGCTAAGTAAGTGATAT
ATATATATATATACTTATTAAAAAAAAAAAAAAAACCTTCTCGAATATTTTCTTTCAGTAAAATACATATGCTTCCTCCGTTTTTTTTT
TTTACTTGCAACACTTTGACTTCTACACTATTCATATACTCTAATTCGACTAGCTCTTATAATTAAAAGTAAAGGAAGTAATATTTA
AATTTTGGAGTATGCATCCCTAAACACTGTTGAATGATGACAATGAAAATAAGTACCATAAGCTTCAAAATGCATATACTAATGTG
GTATTTTGGTCTGCAGGGATACCTCAAAATATCCTCCTTTGATGAAAACAAGCAAGCTCAGCAACTCACTTAGATATCTAACACA
AGCATATTTAGGGCAAGTACCTCTTAAGTCTTAACTAAGTATATGGTTCTTTATATATGTTGTTTGTAAACTCGCTCTTTCATATTT
ACTGATAATATTTACGTTACAGTTACGAAATCCAGATTGGTGTCCAAGATCCTTATGAAGATTGTGTTGCTACAATGAAACTCTAC
ATGAGAATGAGGTCTCAAAATCATAAGGCAGAATATTATCCATTAGCCAGTGACCCACAAAATCGCAACAACTTTGCTCAATGGA
GGGAAGCTGAACTTGAGAAAATGACTCCAGATGAACTATTAGCAATATCAAGGTCCGATTACTATTGCTGGTGCTTAGACTTGAA
GGGATATATGTGA
SEQIDNO: 313
MRTSFHSVHEPMCGVCKKRCRSFESLREHLIGPLPKVECAKIFSSRGCNICLTILDSPYTLRMHRGACQLSRPNGLISSMANMKICDN
MKIDYGRERSPNVVALACKMVGGGNDGTLDICARVCLTDEYDNILFQTYVKPHIPVTNYRFETTGIRPDHLRDAMPLKQVQRKIQDFL
CNGEPVWKIRSRGGKARVLVGHGLDHDLECLGMEYPALMISYEIQMGVQDPYEDCVATMKLYMRMRSQNHKAEYYPLASDPQNRN
NFAQWREAELEKMTPDELLAISRSDYYCWCLDLKGYM
SEQIDNO: 314
ATGAGGACCTCCTTTCACTCTGTTCATGAACCTATGTGTGGTGTTTGCAAAAAGCGTTGTCGATCCTTCGAATCATTAAGAGAAC
ATCTTATTGGACCATTACCAAAGGTGGAATGCGCAAAGATCTTTAGTAGCCGAGGATGCAACATATGCTTGACCATTTTAGATAG
CCCTTACACCCTCAGAATGCATCGAGGAGCATGTCAACTCTCTCGCCCAAATGGACTAATTTCTAGCATGGCAAACATGAAAAT
ATGTGACAATATGAAGATTGATTATGGTCGAGAAAGAAGTCCTAACGTAGTTGCGCTTGCATGCAAAATGGTAGGAGGTGGTAA
TGACGGAACACTTGATATTTGTGCTAGGGTTTGCCTAACTGATGAATATGATAACATTTTGTTTCAAACATATGTTAAGCCTCATA
TTCCAGTGACTAACTATAGATTTGAAACCACGGGTATCAGACCGGATCACTTGAGGGATGCAATGCCATTGAAGCAGGTTCAAA
GAAAAATTCAAGATTTCTTATGCAATGGAGAGCCGGTGTGGAAAATTCGATCAAGAGGAGGAAAGGCTAGGGTTCTTGTTGGCC
ATGGACTAGATCATGACCTTGAGTGCCTAGGAATGGAATATCCCGCATTGATGATTAGTTACGAAATCCAGATGGGTGTCCAAG
ATCCTTATGAAGATTGTGTTGCTACAATGAAACTCTACATGAGAATGAGGTCTCAAAATCACAAGGCGGAATATTATCCATTAGC
TAGTGACCCACAAAATCGTAACAACTTTGCACAATGGAGGGAAGCTGAACTTGAGAAAATGACTCCAGATGAACTATTAGCAATA
TCAAGGTCTGATTACTATTGCTGGTGCTTAGACTTGAAGGGATATATGTGA
SEQIDNO: 315
ATGAGGACCTCCTTTCACTCTGTTCATGAACCTATGTGTGGTGTTTGCAAAAAGCGTTGTCGATCCTTCGAATCATTAAGAGAAC
ATCTTATTGGTAGGTTAATTTATATCTAATAATATAGTTACTATCTGTGTTTCATATTATCTGCAACATCTACTAAAAACAGTTATAT
CATTATCTTTCTATCTCCATTTAACACTCACGAAACAAATTTTGTCTATATAGGACCATTACCAAAGGTGGAATGCGCAAAGATCT
TTAGTAGCCGAGGATGCAACATATGCTTGACCATTTTAGATAGCCCTTACACCCTCAGAATGCATCGAGGAGCATGTCAACTCT
CTCGCCCAAATGTAACGACTTTTTATTAGCTTGGAAAAACAAATGTAACATATTAATAGTATTTCCATTGATAAAAACTTTGTAAAT
TAAAACCTATGCCCTTTTTTATACGCAGGGACTAATTTCTAGCATGGCAAACATGAAAATATGTGACAATATGAAGATTGATTATG
GTCGAGAAAGAAGTCCTAACGTAGTTGCGCTTGCATGCAAAATGGTAGGAGGTGGTAATGACGGAACACTTGATATTTGTGCTA
GGGTTTGCCTAACTGATGAATATGATAACATTTTGTTTCAAACATATGTTAAGCCTCATATTCCAGTGACTAACTATAGGTAACTT
TAATTTTCGACATAAATTTCTCTACTGCTCCCTCCGTTTCATAATTAGAAGTCTATTTCTCTTTATGATGTGAGTAATAGATGTTTG
AAATAGGGTTCTAATTATGAAACGGAGGGAGTATCTTGATTTAAACTATTTTATATGCCGACTCATTCACTTATTGTATTTCGTGT
GTAGATTTGAAACCACGGGTATCAGACCGGATCACTTGAGGGATGCAATGCCATTGAAGCAGGTTCAAAGAAAAATTCAAGATT
TCTTATGCAATGGAGAGCCGGTGTGGAAAATTCGATCAAGAGGAGGAAAGGCTAGGGTTCTTGTTGGCCATGGACTAGATCAT
GACCTTGAGTGCCTAGGAATGGAATATCCCGCATTGATGATTAGGTATGATGAACTACTTTCTCCAATCTCCATTTCTAAGTATTT
GCATGATCAATTGACTATATTTGTCAATGCACTACTTTGACTTTTTTTTTTTTTTTGAGACAATTAATATCTTTACTTATGCTTTATTA
AAATTATATTTTGAAAATATATATTGAGATTAGTTAATTCAATGACAATTAAAATCATGAAATTTATTATACTTTTTATACATTATCAT
TAGTGCTAAAATATGATCAAAGTTAGTATAAGAAGAGTATAAAAAGTCAATAAACGGAGGAAGTATATTGATAATGCATTATCACA
TACAAATAAATGAGTTCATAACTAATTACAGTCATTTGATGGTTTGATTTGATTGTATATAAGTACTCAATATCTCTCTTAAATCAA
ATTTAGATAATATTTGTTGGTTGGGAATGGTAGAATTATATTGAGGAAAGTACAAGGTTGTTACACAAGGGAGATTGTGAGGGTC
CTTGGGGTTTTAAATTTCTTGCAAGGAAACTTAATTGTAGATTTGTTCTATCTAGCTAGCAAGCTAAGTAAGTGATGTATATACTA
TAAAATGATTTATTTTTTTTTTAAAAAAAAAAAAAAAAAAAAAAACCTTTCTCCTTCAGTAAAATAGATATACTTTCTACGTTTTTTTT
TTACTTGCAATGCTTTGACTTTTACAATATTCATACTCTACTTTGACTAGCTCTAATAATTTATTGAATGATGACAATAAAAATAATA
AGTACCATAAGCTTCAAAATGCATGCATATACTAATGTGGTATATTATATTTTTGGTCAACAGGGATACCTCAAAATATCCTCCTT
TGATGAAAACAAGCAAGCTCAGCAATTCACTTAGATATCTAACACAAGCATATTTAGGGCAAGTACCTCTTAACTAAGTATACGG
TTCTTTATATATTTTGTTAGTAAACTCGCTCTTTCATATTTACTGATAATATTTATGTTACAGTTACGAAATCCAGATGGGTGTCCA
AGATCCTTATGAAGATTGTGTTGCTACAATGAAACTCTACATGAGAATGAGGTCTCAAAATCACAAGGCGGAATATTATCCATTA
GCTAGTGACCCACAAAATCGTAACAACTTTGCACAATGGAGGGAAGCTGAACTTGAGAAAATGACTCCAGATGAACTATTAGCA
ATATCAAGGTCTGATTACTATTGCTGGTGCTTAGACTTGAAGGGATATATGTGA
SEQIDNO: 316
MESRVESSETLRNKCAACYRQFNRIEHLVEHMRTSYHSSHEPMCGICKKHCRSFESLREHLIGPLPKQECRNIFITRGCKICLAILESP
YALRVHQDRCQLSGGNAGLLGRFANLGIRDNTEXGSTRGTQVVALACKMVGGGSDGSLDLCAKVCLTDEYDNIIFQSYVKPLLPVTN
YRYETTGIRPEYLRDAMPLRQVQKKIQDFLCNGEPMWKIRPRGGKARILVGHGLDHDLDSLQFEYPQGYCKIPSIDENKQAQQLTQV
SHTSISWASIQDPYEDCVATMKLYMRMRSQVHKVEAYPLASDPQNRNNFAAWRQNELERMSPDQMLEISRSDYYCWCLDS
SEQIDNO: 317
ATGGAGTCTAGAGTCGAATCATCAGAAACTCTGAGGAACAAGTGTGCAGCATGCTATCGACAGTTCAATCGGATCGAGCACTTA
GTGGAGCACATGAGAACCTCATACCACTCATCTCATGAACCCATGTGTGGAATTTGTAAGAAGCATTGCAGATCTTTTGAATCTC
TCAGAGAACATCTTATAGGGCCACTGCCAAAACAAGAGTGCAGAAACATATTCATCACTAGAGGATGCAAAATCTGCTTAGCCA
TCCTCGAAAGCCCGTATGCTCTTAGGGTTCACCAGGACAGATGCCAACTCTCTGGTGGAAATGCTGGATTACTTGGTCGCTTTG
CTAATCTGGGCATTCGTGACAATACTGAARGTGGTAGCACAAGAGGCACTCAAGTTGTTGCACTGGCTTGCAAAATGGTTGGTG
GAGGCAGCGATGGCTCCCTGGATCTCTGCGCAAAGGTCTGCCTCACTGATGAATATGACAACATCATCTTCCAGTCTTATGTCA
AGCCACTGCTTCCAGTCACAAACTATAGGTATGAAACAACTGGTATTCGTCCTGAATACTTGAGGGATGCAATGCCTTTGAGGC
AAGTGCAAAAGAAGATTCAAGATTTCCTCTGCAATGGGGAACCGATGTGGAAGATTCGACCCCGAGGTGGAAAAGCTAGGATT
CTTGTGGGCCACGGTTTAGATCATGACCTTGACTCCTTGCAGTTTGAATACCCACAAGGATACTGCAAAATACCCTCCATTGATG
AAAACAAGCAAGCTCAGCAACTCACTCAAGTATCTCACACAAGCATATCTTGGGCAAGCATTCAAGATCCTTATGAGGATTGTGT
TGCAACAATGAAGCTCTACATGAGAATGAGATCCCAAGTTCACAAAGTAGAGGCATACCCACTCGCTTCTGACCCGCAAAACCG
GAATAACTTCGCGGCGTGGCGCCAAAATGAGCTTGAGAGGATGAGCCCAGATCAAATGCTGGAAATCTCAAGGTCTGATTACT
ACTGCTGGTGCCTGGACTCTTAG
SEQIDNO: 318
ATGGAGTCTAGAGTCGAATCATCAGAAACTCTGAGGTTATCTCTCTCTCTCTCAACACACACGTAAATATCASTGCATGCACATT
TCTTTCAGCTTAATTAAGTTGAAATTAATTTTTCGATGAATCATGGATGATTAGTATGTAAACTAATGATGACAATATTATATATGT
GACGATTAATCAGGAACAAGTGTGCAGCATGCTATCGACAGTTCAATCGGATCGAGCACTTAGTGGAGCACATGAGAACCTCAT
ACCACTCATCTCATGAACCCATGTGTGGAATTTGTAAGAAGCATTGCAGATCTTTTGAATCTCTCAGAGAACATCTTATAGGTAA
CTAAAACAATTTATGCAGAACTGAGCTTATAGCCAAGTWAACTATTACWGTGKTTTCCTCTTTCTGCATCCAAGTTCAAATCCTT
CTCTCCGCTTAGATTAATTTAGAGTAAACTATATCACTTATATAAAAAACAGTTATCAATTACATAAACTAATATCCCAAATTTCTTT
TGCAAATCATAGTTCTATWTTCTTTTTCCAGGGCCACTGCCAAAACAAGAGTGCAGAAACATATTCATCACTAGAGGATGCAAAA
TCTGCTTAGCCATCCTCGAAAGCCCGTATGCTCTTAGGGTTCACCAGGACAGATGCCAACTCTCTGGTGGAAATGCTGTATTAA
TCCTCACACACTAGCTTATTAATCATGAACTTTTTGCCGAAACCCTATTTGTTATAAATTAAAATTCATTCCATATTGTATATTTCAG
GGATTACTTGGTCGCTTTGCTAATCTGGGCATTCGTGACAATACTGAARGTGGTAGCACAAGAGGCACTCAAGTTGTTGCACTG
GCTTGCAAAATGGTTGGTGGAGGCAGCGATGGCTCCCTGGATCTCTGCGCAAAGGTCTGCCTCACTGATGAATATGACAACAT
CATCTTCCAGTCTTATGTCAAGCCACTGCTTCCAGTCACAAACTATAGGTATATATGCATTTATATAGAGATTTATACTAACTAGT
ATGTACGTAAGTAGCGAATATATATAAGAAAGTGGTTTCCGTGGTCTACTCTTTATTCCTGACCGTCAGATGGAACAAAACAAAA
GGCAAAGATAGGGTGGAAATYGGACAAAGATTGTCTGCCCTCYCACTTCCGATGCCCTCYTGTTTTGTATGRTCACGGTTAAGY
CACGTTAACATTTTATATTGTTTTTTTATAGAGATAATAAGACAAAAATGAATAGTAATATAAAATGTTGATGTGGCTTAACCGTGA
CCACATAAACAGGAGGGCAYGRGAGGGCACCGGAAGTGGGAGGGCAGACAATCCTTGTCCGTCGAAATCATACTTTCTCAGTA
AAGATGTCATTTTTTTATGATTTAGCGATTTGACCGACCATTTTGTTGTATACTTGTATAATACAGGTATGAAACAACTGGTATTCG
TCCTGAATACTTGAGGGATGCAATGCCTTTGAGGCAAGTGCAAAAGAAGATTCAAGATTTCCTCTGCAATGGGGAACCGATGTG
GAAGATTCGACCCCGAGGTGGAAAAGCTAGGATTCTTGTGGGCCACGGTTTAGATCATGACCTTGACTCCTTGCAGTTTGAATA
CCCACAAGTAATGATTAGGTAATTCAACTTATTTTTCTTTCATCCAAACAAAAAACCTATTTATCATTTTATGTACAGAGAAAATTG
TTATTGACAATTAAAAAGTCTTAAAATAAATAAGATCACATATTGAGTTGCACACCTATTAATTGGCAGCAAATCATTTGTGTATGT
ACACACACGCGCACATCGTTTGTGTATGTACACACACACGCGCACACACACACGCGCGCACACACACAACCTTCCATTCCGAAT
TTCAATTATAATTTTAGTTCTAAATGTGACTGACTGTGTATAGGGATACTGCAAAATACCCTCCATTGATGAAAACAAGCAAGCTC
AGCAACTCACTCAAGTATCTCACACAAGCATATCTTGGGCAAGTACCCAACCCTACTATTAATTCCCTATATATAGATCAATTCTA
GCTACAAATAAGTTATTACTAATGCAATTTTCACATATAATCTATATAGGTATGAAATTCAAACAGGCATTCAAGATCCTTATGAG
GATTGTGTTGCAACAATGAAGCTCTACATGAGAATGAGATCCCAAGTTCACAAAGTAGAGGCATACCCACTCGCTTCTGACCCG
CAAAACCGGAATAACTTCGCGGCGTGGCGCCAAAATGAGCTTGAGAGGATGAGCCCAGATCAAATGCTGGAAATCTCAAGGTC
TGATTACTACTGCTGGTGCCTGGACTCTTAG
SEQIDNO: 319
MEYKVESTETLRNKCAACYRQFNRIEHLVEHMRTSYHSAHEPMCGICKKHCKYFESLREHLIGPLPKQECRNIFSTRGCKICLAILESP
DYLVALLIWGIRDNTESGSTGGTQVVALACKMVGGGSDGSLDLCAKVCLTDEYDNIIFQSYVMPLIPVTNYRYETTGIRPEYLRDAIPL
RQVQKKIQDFLCNGEPKWKIRLRSGRARILVGHGLDHDLDSLQVEYPQGYCKISSIVEIKQAQQLPQVSHTSISWASIQDPYEDCVAT
MKIYKRMRSQVHKVEAHPLASDTQNRNNFAAWRQNELERMSPDQMMEISRSDYNCWCLDSYSG
SEQIDNO: 320
ATGGAGTATAAAGTTGAATCAACAGAAACTCTGAGGAACAAGTGTGCAGCATGCTATCGACAATTCAATAGAATCGAGCACTTA
GTGGAGCACATGAGAACCTCATACCACTCAGCTCATGAACCCATGTGTGGAATTTGTAAGAAGCATTGCAAATATTTTGAATCTC
TCAGGGAACATCTTATAGGGCCATTGCCAAAACAAGAATGCAGAAATATATTCAGCACCAGAGGATGCAAAATCTGCTTAGCCA
TCCTCGAAAGCCCGGATTACTTGGTCGCTTTGCTAATCTGGGGCATTCGTGACAATACTGAAAGTGGTAGCACAGGAGGCACT
CAAGTTGTTGCACTTGCTTGCAAAATGGTTGGTGGTGGCAGCGATGGCTCACTGGATCTCTGCGCAAAGGTCTGCCTCACTGAT
GAATATGACAACATCATCTTTCAGTCTTATGTCATGCCACTGATTCCAGTCACAAACTATAGATATGAAACAACTGGTATTCGCCC
TGAATACTTGAGGGATGCAATCCCTTTGAGGCAAGTGCAAAAGAAGATCCAAGACTTCCTCTGCAATGGGGAACCAAAGTGGAA
GATTCGACTCCGAAGTGGAAGAGCTAGGATTCTTGTGGGTCACGGTTTGGATCATGACCTTGACTCTCTGCAGGTTGAATATCC
ACAAGGATACTGCAAAATATCCTCCATTGTTGAAATCAAGCAAGCTCAGCAACTCCCTCAAGTATCTCACACAAGCATATCTTGG
GCAAGCATTCAAGATCCTTATGAGGATTGTGTTGCAACAATGAAGATCTACAAGAGAATGAGATCCCAAGTTCACAAAGTAGAG
GCACACCCGCTCGCTTCTGACACTCAAAACCGGAATAACTTTGCGGCGTGGCGCCAAAATGAGCTTGAGAGGATGAGCCCAGA
TCAAATGATGGAAATCTCAAGGTCTGATTACAACTGCTGGTGCCTGGACTCTTATAGTGGCTGA
SEQIDNO: 321
ATGGAGTATAAAGTTGAATCAACAGAAACTCTGAGGTTATCTCTCTCTCTCTCTCTCAACACATACAGAAATATCACTGCATTCAC
ATTTCTTTCAGCTTAATTAAGTTGTAACTTGTAAGTAATTTTTTTGATTAATCATGGATGATTAGTATATAAACTAATGAGAATTATA
TATATGCGGTGATTAATCAGGAACAAGTGTGCAGCATGCTATCGACAATTCAATAGAATCGAGCACTTAGTGGAGCACATGAGA
ACCTCATACCACTCAGCTCATGAACCCATGTGTGGAATTTGTAAGAAGCATTGCAAATATTTTGAATCTCTCAGGGAACATCTTA
TAGGTAACTAAAACAAACTATGCAGATCAACTGAGCGTAGCCAAGTTGACTATAAACTGTGTTTTCTCTTTCTGCATCAAAGTTCA
AATCCTTCTCTCCATAGCTTAAATTAATTTAGAGTCAACTATATCTCTTATACAAAAAAACAGTTATTAATTACATAATCCCAAATTT
CTTTTGCAAATAATAATCCTATTATTTCTATTTTCAGGGCCATTGCCAAAACAAGAATGCAGAAATATATTCAGCACCAGAGGATG
CAAAATCTGCTTAGCCATCCTCGAAAGCCCGTATGCTCTTAGGGTTCACCAGGACAGATGCCAACTCTCTGGCGGCAATGCTGT
ATTAATCAGCACACACTACCTTATTAATTATGACCTTTTGCCCAAACCCTATTTGCTATAAATTTAAATTGATTCCATATAATGCAT
TTCAGGGATTACTTGGTCGCTTTGCTAATCTGGGGCATTCGTGACAATACTGAAAGTGGTAGCACAGGAGGCACTCAAGTTGTT
GCACTTGCTTGCAAAATGGTTGGTGGTGGCAGCGATGGCTCACTGGATCTCTGCGCAAAGGTCTGCCTCACTGATGAATATGA
CAACATCATCTTTCAGTCTTATGTCATGCCACTGATTCCAGTCACAAACTATAGGTATATATGCATTTATATGAAGAATTATACTAA
CTAATATGTAACGTATGTAGCGTATATATGTAGGGGAATGATTTCAGGGTCTCTTCTTTATTTCTAACTGTCGGATGGAACAAAAC
AAAAGACGAAGATATTGTGAAAATTGTACTTCCTACAATAAAGATGTTGTTGCTTTAAGAGAATATGGTCATCATTTCGGAGTGTG
ATTTAGCGATTGAACGGGTTCATTTTTTATATACATGACCGATCATTTTGCTTTATACTTGTATATATACAGATATGAAACAACTGG
TATTCGCCCTGAATACTTGAGGGATGCAATCCCTTTGAGGCAAGTGCAAAAGAAGATCCAAGACTTCCTCTGCAATGGGGAACC
AAAGTGGAAGATTCGACTCCGAAGTGGAAGAGCTAGGATTCTTGTGGGTCACGGTTTGGATCATGACCTTGACTCTCTGCAGGT
TGAATATCCACAAGTAATGATTAGGTAATTCAACTTATTTTTCTTTCATCCAAACAAAAAATATCCATTTTATAAAATCATCAGACT
CTCTTCGATCAATGAAAAGAAAATAAAAGACAACGAAGTGCACAATGACTATTTTAAAGTACTAATTAACAATTATCTATATATATT
TATAAAAGTATAGGATCACATATATACACATACAACTTTCCATTGCGAATTTCAATTGTAATTCTAGTTCTAAACGTGACTGACTGT
GTATAGGGATACTGCAAAATATCCTCCATTGTTGAAATCAAGCAAGCTCAGCAACTCCCTCAAGTATCTCACACAAGCATATCTT
GGGCAAGTACCCAACCCTACTATTAGTTCCCTATATATATAAATATAAATTCTAGCTACATATTAGTACTAATGCAATTTTACATAT
AATCTTATTAGGTATGACATTCAAACAGGCATTCAAGATCCTTATGAGGATTGTGTTGCAACAATGAAGATCTACAAGAGAATGA
GATCCCAAGTTCACAAAGTAGAGGCACACCCGCTCGCTTCTGACACTCAAAACCGGAATAACTTTGCGGCGTGGCGCCAAAAT
GAGCTTGAGAGGATGAGCCCAGATCAAATGATGGAAATCTCAAGGTCTGATTACAACTGCTGGTGCCTGGACTCTTATAGTGGC
TGA
SEQIDNO: 322
MDYRTESSETLRNKCAACFRQFNRLEHLVEHMRTSYHSVHEPVCGICKKHCRSFESLREHLIGPLPKQECKNVFNNRGCRFCLAILD
GPNARRLHQERCQMVSSATTGLNARLANLGVRDSLTIDNTHTRDPQVVAIACKMVGGGSDGSLDLCGRVCIIDEYENIIFHAYVKPPIP
VTSYRYETTGIRPEHLRDAIPLKQVQRKIQDFLCNGEPMWKIRPRGSRARILVGHGLDHDLDRLQVEYPAIMTRDTAKYPPLMKTSKL
SNSLKYLTQAYLGYDIQIGIQDPYDDCVATMRLYMRMKSQAHKREDYPLASDPQNRNNYASWRQTELERMSPEEMLAISRSDYYCW
CLDR
SEQIDNO: 323
ATGGACTACAGAACGGAGTCTTCAGAAACCCTAAGGAACAAGTGTGCAGCATGCTTCAGACAGTTCAACAGATTGGAGCACCT
GGTTGAGCACATGAGAACTTCATATCATTCGGTTCATGAACCCGTGTGTGGAATTTGTAAGAAACATTGCAGATCTTTTGAATCT
CTGAGGGAACATCTTATAGGGCCACTGCCAAAGCAAGAGTGCAAAAACGTATTCAACAACAGAGGATGCAGATTCTGTTTAGCC
ATCCTCGATGGCCCTAACGCTCGTAGGCTTCACCAAGAAAGATGCCAGATGGTCTCAAGTGCAACCACTGGACTGAATGCTCG
CTTAGCCAACTTAGGCGTTCGAGACAGCCTAACCATTGACAATACTCATACAAGAGATCCGCAAGTCGTAGCAATAGCTTGCAA
AATGGTTGGTGGAGGCAGTGATGGCTCTCTTGACCTTTGCGGAAGGGTCTGCATCATTGATGAATATGAAAACATTATCTTCCAT
GCTTATGTTAAGCCGCCGATTCCGGTCACAAGTTATAGGTATGAAACAACAGGCATTAGGCCAGAACATCTCCGCGATGCAATT
CCACTGAAGCAAGTTCAAAGGAAGATTCAAGATTTCCTTTGCAATGGGGAACCAATGTGGAAAATTAGACCTAGAGGTAGCAGA
GCTAGGATTCTAGTGGGTCATGGCTTGGATCACGATCTTGATCGTTTGCAAGTAGAATATCCAGCAATAATGACAAGGGATACT
GCAAAATATCCTCCACTGATGAAGACAAGCAAGCTTAGCAATTCTCTCAAATATTTAACACAAGCATACCTAGGATATGACATTC
AAATTGGTATACAAGACCCGTACGATGATTGTGTTGCAACAATGAGGTTGTACATGAGAATGAAATCTCAAGCTCATAAGAGAGA
GGACTATCCACTTGCTTCTGACCCGCAAAACAGAAATAACTATGCATCATGGAGGCAAACTGAGCTTGAGAGGATGAGTCCTGA
AGAAATGCTAGCAATCTCAAGGTCTGATTACTACTGTTGGTGCTTGGACCGATGATCTGCAAATGGTAGGGCGTGGTAGCCAGA
GGAACAAAATTGATGCGGGATATTGAAGAGGCTTCTAGTTAAAC
SEQIDNO: 324
ATGGACTACAGAACGGAGTCTTCAGAAACCCTAAGGTATAGACTACCCTCGTACATCCCTTTTTACTCCTCAACACCCACAATCA
GCATATGATCTTTCTACATGAAATCGATTGAATAAAATTGTTCCGTAATAATTAGTAACTTGATTGATCTTAATTTATTACCACAAT
CAGGAACAAGTGTGCAGCATGCTTCAGACAGTTCAACAGATTGGAGCACCTGGTTGAGCACATGAGAACTTCATATCATTCGGT
TCATGAACCCGTGTGTGGAATTTGTAAGAAACATTGCAGATCTTTTGAATCTCTGAGGGAACATCTTATAGGTAAATTTACTTTCA
ATTTGTCCATGGATTATTTTGTAACTTCCTATATAAGTTTAGTTTATCAATCAGCAATCCTGATATTCGCAATACAATTAATTACAG
GGCCACTGCCAAAGCAAGAGTGCAAAAACGTATTCAACAACAGAGGATGCAGATTCTGTTTAGCCATCCTCGATGGCCCTAACG
CTCGTAGGCTTCACCAAGAAAGATGCCAGATGGTCTCAAGTGCAACCACTGTATTACTCTTTTAATTTACCTTGCAATACTTATTG
TTAAATATTCATACTTAATTAATCAGATCAAATTGATCTTATTGTCAACTTCACGTGTTCTAGGGACTGAATGCTCGCTTAGCCAA
CTTAGGCGTTCGAGACAGCCTAACCATTGACAATACTCATACAAGAGATCCGCAAGTCGTAGCAATAGCTTGCAAAATGGTTGG
TGGAGGCAGTGATGGCTCTCTTGACCTTTGCGGAAGGGTCTGCATCATTGATGAATATGAAAACATTATCTTCCATGCTTATGTT
AAGCCGCCGATTCCGGTCACAAGTTATAGGTACCAATATGCATATACTTATGTATATATATACACATTTTGATGATTTCAGGAGCA
CAAATTAACTGTACTTTTAGTAAATCTTACATTCTTGAAACTTGTGTCCTAGGTATGAAACAACAGGCATTAGGCCAGAACATCTC
CGCGATGCAATTCCACTGAAGCAAGTTCAAAGGAAGATTCAAGATTTCCTTTGCAATGGGGAACCAATGTGGAAAATTAGACCT
AGAGGTAGCAGAGCTAGGATTCTAGTGGGTCATGGCTTGGATCACGATCTTGATCGTTTGCAAGTAGAATATCCAGCAATAATG
ACAAGGTAAATTTAAGTTGAAATTAAACATGCCAGCCACAAAACTTTATTACCATGAAACTCTTATGATAATTAATCTGCTAAGAA
AATGTATACATAATGCTCTTATCATTATCAGGGATACTGCAAAATATCCTCCACTGATGAAGACAAGCAAGCTTAGCAATTCTCTC
AAATATTTAACACAAGCATACCTAGGGTAAGTGCCATATATGTTTGTACATTTTTTTCCCCCAAACTTTCTAGATAAATGATTTTTA
TATATTACTCTCCATAATTCTCTATGCAGATATGACATTCAAATTGGTATACAAGACCCGTACGATGATTGTGTTGCAACAATGAG
GTTGTACATGAGAATGAAATCTCAAGCTCATAAGAGAGAGGACTATCCACTTGCTTCTGACCCGCAAAACAGAAATAACTATGCA
TCATGGAGGCAAACTGAGCTTGAGAGGATGAGTCCTGAAGAAATGCTAGCAATCTCAAGGTCTGATTACTACTGTTGGTGCTTG
GACCGATGATCTGCAAATGGTAGGGCGTGGTAGCCAGAGGAACAAAATTGATGCGGGATATTGAAGAGGCTTCTAGTTAAAC
SEQIDNO: 325
MDTEAELPKRSTARHKCVACYKQFKRKDHLIEHMKISYHSVHQPKCAVCQKLSKSFESLREHLTGPLSKAHCSGIFSDRGCNLCMNIF
DSPSSLIKHKEACSLSAPVPFEKTLSNAESQKKISGAIDEKRTCRGPKAVAMDCEMVGGGSNGTLDLCARVCLVDEDENVIFHTYVQP
QLPVTNYRYEVTGLTEEDIKNAMPLKEVKDKILEILNNGESTGRLMLDDGKARLLVGHGLEHDLDSLRMNYPDHMLRDTAKYRPLMKT
NLVSHSLKYLTRTYLGYDIQSGVHDPYEDCVSVMRLYKRFRRQDHQVEEIGNQNTTGSFDSYKYKELEKMSPNELYQISKSDYRCW
CLD
SEQIDNO: 326
TGAAATTTCCGAGTCATCAGGTGTTGAAATGGACACTGAAGCGGAGCTTCCGAAAAGGTCAACGGCAAGGCACAAATGTGTTG
CGTGCTACAAACAATTTAAGCGAAAGGATCATCTTATTGAGCACATGAAAATCTCATACCATTCAGTTCATCAGCCAAAATGTGC
TGTCTGTCAAAAACTCTCTAAATCTTTTGAATCTCTGCGGGAACATCTTACTGGTCCATTATCAAAAGCACATTGTTCGGGGATTT
TCTCTGACAGAGGGTGCAATCTTTGTATGAATATTTTTGATAGCCCCAGTTCTCTTATCAAGCATAAAGAAGCATGTTCCCTTTCT
GCACCTGTTCCCTTTGAAAAAACACTGTCTAATGCAGAATCTCAAAAGAAAATTTCAGGGGCAATTGATGAAAAGCGCACATGCA
GAGGTCCCAAAGCAGTTGCTATGGACTGTGAAATGGTTGGTGGTGGAAGCAATGGAACACTTGATCTTTGTGCTAGAGTTTGCC
TTGTTGATGAAGATGAGAATGTAATTTTTCACACATATGTTCAACCCCAACTTCCTGTCACTAACTACAGATATGAAGTAACTGGG
TTGACTGAAGAGGATATAAAAAATGCCATGCCTCTTAAAGAAGTGAAGGATAAAATTTTGGAAATTTTGAACAATGGAGAGTCTA
CCGGGAGGCTTATGTTGGATGATGGAAAGGCCAGACTTCTTGTGGGTCATGGTCTAGAGCATGATTTGGATAGCTTGAGGATG
AATTACCCTGATCACATGCTGAGGGATACTGCAAAATATCGTCCATTGATGAAGACAAATCTGGTTAGCCACTCGCTCAAGTACC
TCACCAGAACGTATCTAGGGTATGATATACAGTCTGGGGTGCATGATCCATACGAAGATTGTGTATCTGTCATGAGACTGTACA
AGAGATTCCGCAGACAAGATCATCAGGTAGAAGAAATTGGAAACCAAAATACTACCGGCAGTTTTGATTCTTACAAATACAAGGA
GCTTGAGAAGATGAGCCCAAATGAACTGTACCAGATATCAAAATCAGACTATAGATGCTGGTGCTTGGATTAGTATGGAGCCTT
AGCTTGTGTACCTCTGCTTTCTTCATCTCTATGTATATAGCAATTTTTAT
SEQIDNO: 327
TGAAATTTCCGAGTCATCAGGTGTTGAAATGGACACTGAAGCGGAGCTTCCGAAAAGGTCAACGGCAAGGTAACACTCTCTCAA
TTACATTTTGCTAAAAGAGGCAACGGTCGATACAGCAAAAATGAGGGTTTTGACTTTTTGAGAATAATTAGTTAATGCAATTTTGA
GCTATTTACCTTTTTGGGTCATCAGAATTTACCCTTCTTATAGTTATGTACTCGTCTTCTTAGCTTTGTAAAGTCTAATGGGTGATT
TTGTCAGCTCTGGTGATTGCATTTGCTGTGTCGGTGCTTCGTTGTTTTTGTGTGCTACACTTGTTGAGACAATGGTCATGGGTAA
TGTGACTCCTGTGCTTACATTACTTTCAGGCACAAATGTGTTGCGTGCTACAAACAATTTAAGCGAAAGGATCATCTTATTGAGC
ACATGAAAATCTCATACCATTCAGTTCATCAGCCAAAATGTGCTGTCTGTCAAAAACTCTCTAAATCTTTTGAATCTCTGCGGGAA
CATCTTACTGGTAAAATATATTTTCATGTTCATTCTGATTGGTCCATTCGTCAGTTGCTTATTTTGTTTTCCTTCTTATAGGTTCTTA
TTTTATTTTCCTTCTTATAGGTCCATTATCAAAAGCACATTGTTCGGGGATTTTCTCTGACAGAGGGTGCAATCTTTGTATGAATA
TTTTTGATAGCCCCAGTTCTCTTATCAAGCATAAAGAAGCATGTTCCCTTTCTGCACCTGTTCCCTTTGTAAGTTGATTATTGAAT
TTTTAGGGTTGCCGCTAGGAAACTTCTAGGATTGTTATCTCATGTAGTTTTTAAACCTTTTGACAATCAATTGCAGAACATCTGAT
CATTTCCTAGACATTCACACTTTGAATATTTTGTTGATTGCCGCAGGAAAAAACACTGTCTAATGCAGAATCTCAAAAGAAAATTT
CAGGGGCAATTGATGAAAAGCGCACATGCAGAGGTCCCAAAGCAGTTGCTATGGACTGTGAAATGGTTGGTGGTGGAAGCAAT
GGAACACTTGATCTTTGTGCTAGAGTTTGCCTTGTTGATGAAGATGAGAATGTAATTTTTCACACATATGTTCAACCCCAACTTCC
TGTCACTAACTACAGGCATTTCCCTGCCCTACATTGTTTTTGATTCTGCAAATTAATCATTAGGAAGAACCTGCTGATCATTGATA
TATCTTCGTAAATAATTTCAGATATGAAGTAACTGGGTTGACTGAAGAGGATATAAAAAATGCCATGCCTCTTAAAGAAGTGAAG
GATAAAATTTTGGAAATTTTGAACAATGGAGAGTCTACCGGGAGGCTTATGTTGGATGATGGAAAGGCCAGACTTCTTGTGGGT
CATGGTCTAGAGCATGATTTGGATAGCTTGAGGATGAATTACCCTGATCACATGCTGAGGTAATATAAATATTTTAAAGATTTTTG
ACCTGTCTAATTGATTTAAGTTCACTTTCATTGAAAGATACATGATAAAAAGGATGGATTGTTATTTGAATCGGACAAATGTCATA
ATATGGTTCAAATCACCCACTCAGCTGGTTATCTATGTTCAATTCAAAGTAAAATGTGTTTATAGATCTTCTCAATCTGCAATACA
ATCAAACTTGATTGTGATTTGGGATCTGGAAATGAGAATCACACTTCTTTTAAAACAATCTTGTGATGTATCTATGATTACCTCCTT
TACCTTATTTCCTGGGCTTAAACTTCAAGTCTTCTCCAGCAAAGGCCACCAAACTCGAGCAGCCACAGTTATGGTAAAAGTTTTG
ATGAAATTTCTACTAGTAGCATTTAAACTTAGCTTCAAAAGTTTTACTTGGAGAAAAATAACTCTGAACTCTGTTTGTATCTAGTTG
CATTGGTTTCATAAGTTTCACTTGGTGAAAAAATAAACCTGAACTCTGTTTGTATCTGGTTTCATGTGTTTTGGCTCTCACAGTTTT
GATTTCAATATTTTAAGACATTTTCGATTATCATATATTTTGTCTATGCAGCTGTATTGTTGCATTGAAGCATGGCCTTTTTTTTTTG
CGCCTTAAAATAACCTTTTCTTTGTGTAGGGATACTGCAAAATATCGTCCATTGATGAAGACAAATCTGGTTAGCCACTCGCTCA
AGTACCTCACCAGAACGTATCTAGGGCAAGTTATTTTTGCTTTCAGCACCCATCCTCATCACTAACACCCCCACCCCCTCTCCCT
TTAATTTTTTAAATTTTCTTTGTTATCCTGAAGTTTCTTGTTCATGTGGAGCTGCTATTGATATTTCTTTTCTTTTACAAGGGAAAAA
GGAAAGAGGAAACTTTTAACTGATTCTAACATTTCACTATTCCACTGTTAAACTATAAAGGTTCTTGGATGCTGTTTATGTTAGAA
AGTCTCGTAACTTTTTTAAAATTTATAGTTGATGTGATAGGGGAAGCAAGAATTCCTAAAGAAGGTCAAAGAAGAAATTTAGTAAA
ATGAAAGGAAAACAGTCTTTTGTTCCCATGCAATATCTATATGCATTTTATGATTTCAAATAACACGTCGGCAGAGTATGGTATGG
TCGGCCACTTGGTGTCTATTCAATGTTTGGAGTTCTGCGATCACTAATGTAATCTGTAAGAAGGATAGAAAATCACCGTATTTATT
TGCTTCACAGGTATGATATACAGTCTGGGGTGCATGATCCATACGAAGATTGTGTATCTGTCATGAGACTGTACAAGAGATTCC
GCAGACAAGATCATCAGGTAGAAGAAATTGGAAACCAAAATACTACCGGCAGTTTTGATTCTTACAAATACAAGGAGCTTGAGAA
GATGAGCCCAAATGAACTGTACCAGATATCAAAATCAGACTATAGATGCTGGTGCTTGGATTAGTATGGAGCCTTAGCTTGTGTA
CCTCTGCTTTCTTCATCTCTATGTATATAGCAATTTTTAT
SEQIDNO: 328
MDTEAELPKRSTARHKCVACYKQFKRKDHLIEHMKISYHSVHQPKCAVCQKLSKSFESLREHLTGPLSKAHCSGIFSDRGCNLCMNIF
DSPSSLIKHKEACSLSAPVPFKISGAIDEKRTCRGPKAVAMDCEMVGGGSNGTLDLCARVCLVDEDENVIFHTYVQPQLPVTNYRYEV
TGLTEEDIKNAMPLKEVKDKILEILNNGESTGRLMLDDGKARLLVGHGLEHDLDSLRMNYPDHMLRDTAKYRPLMKTNLVSHSLKYLT
RTYLGYDIQSGVHDPYEDCVSVMRLYKRFRRQDHQVEEIGNQNTTGSFDSYKYKELEKMSPNELYQISKSDYRCWCLD
SEQIDNO: 329
TGAAATTTCCGAGTCATCAGGTGTTGAAATGGACACTGAAGCGGAGCTTCCGAAAAGGTCAACGGCAAGGCACAAATGTGTTG
CGTGCTACAAACAATTTAAGCGAAAGGATCATCTTATTGAGCACATGAAAATCTCATACCATTCAGTTCATCAGCCAAAATGTGC
TGTCTGTCAAAAACTCTCTAAATCTTTTGAATCTCTGCGGGAACATCTTACTGGTCCATTATCAAAAGCACATTGTTCGGGGATTT
TCTCTGACAGAGGGTGCAATCTTTGTATGAATATTTTTGATAGCCCCAGTTCTCTTATCAAGCATAAAGAAGCATGTTCCCTTTCT
GCACCTGTTCCCTTTAAAATTTCAGGGGCAATTGATGAAAAGCGCACATGCAGAGGTCCCAAAGCAGTTGCTATGGACTGTGAA
ATGGTTGGTGGTGGAAGCAATGGAACACTTGATCTTTGTGCTAGAGTTTGCCTTGTTGATGAAGATGAGAATGTAATTTTTCACA
CATATGTTCAACCCCAACTTCCTGTCACTAACTACAGATATGAAGTAACTGGGTTGACTGAAGAGGATATAAAAAATGCCATGCC
TCTTAAAGAAGTGAAGGATAAAATTTTGGAAATTTTGAACAATGGAGAGTCTACCGGGAGGCTTATGTTGGATGATGGAAAGGC
CAGACTTCTTGTGGGTCATGGTCTAGAGCATGATTTGGATAGCTTGAGGATGAATTACCCTGATCACATGCTGAGGGATACTGC
AAAATATCGTCCATTGATGAAGACAAATCTGGTTAGCCACTCGCTCAAGTACCTCACCAGAACGTATCTAGGGTATGATATACAG
TCTGGGGTGCATGATCCATACGAAGATTGTGTATCTGTCATGAGACTGTACAAGAGATTCCGCAGACAAGATCATCAGGTAGAA
GAAATTGGAAACCAAAATACTACCGGCAGTTTTGATTCTTACAAATACAAGGAGCTTGAGAAGATGAGCCCAAATGAACTGTACC
AGATATCAAAATCAGACTATAGATGCTGGTGCTTGGATTAGTATGGAGCCTTAGCTTGTGTACCTCTGCTTTCTTCATCTCTATGT
ATATAGCAATTTTTAT
SEQIDNO: 330
TGAAATTTCCGAGTCATCAGGTGTTGAAATGGACACTGAAGCGGAGCTTCCGAAAAGGTCAACGGCAAGGTAACACTCTCTCAA
TTACATTTTGCTAAAAGAGGCAACGGTCGATACAGCAAAAATGAGGGTTTTGACTTTTTGAGAATAATTAGTTAATGCAATTTTGA
GCTATTTACCTTTTTGGGTCATCAGAATTTACCCTTCTTATAGTTATGTACTCGTCTTCTTAGCTTTGTAAAGTCTAATGGGTGATT
TTGTCAGCTCTGGTGATTGCATTTGCTGTGTCGGTGCTTCGTTGTTTTTGTGTGCTACACTTGTTGAGACAATGGTCATGGGTAA
TGTGACTCCTGTGCTTACATTACTTTCAGGCACAAATGTGTTGCGTGCTACAAACAATTTAAGCGAAAGGATCATCTTATTGAGC
ACATGAAAATCTCATACCATTCAGTTCATCAGCCAAAATGTGCTGTCTGTCAAAAACTCTCTAAATCTTTTGAATCTCTGCGGGAA
CATCTTACTGGTAAAATATATTTTCATGTTCATTCTGATTGGTCCATTCGTCAGTTGCTTATTTTGTTTTCCTTCTTATAGGTTCTTA
TTTTATTTTCCTTCTTATAGGTCCATTATCAAAAGCACATTGTTCGGGGATTTTCTCTGACAGAGGGTGCAATCTTTGTATGAATA
TTTTTGATAGCCCCAGTTCTCTTATCAAGCATAAAGAAGCATGTTCCCTTTCTGCACCTGTTCCCTTTGTAAGTTGATTATTGAAT
TTTTAGGGTTGCCGCTAGGAAACTTCTAGGATTGTTATCTCATGTAGTTTTTAAACCTTTTGACAATCAATTGCAGAACATCTGAT
CATTTCCTAGACATTCACACTTTGAATATTTTGTTGATTGCCGCAGGAAAAAACACTGTCTAATGCAGAATCTCAAAAGAAAATTT
CAGGGGCAATTGATGAAAAGCGCACATGCAGAGGTCCCAAAGCAGTTGCTATGGACTGTGAAATGGTTGGTGGTGGAAGCAAT
GGAACACTTGATCTTTGTGCTAGAGTTTGCCTTGTTGATGAAGATGAGAATGTAATTTTTCACACATATGTTCAACCCCAACTTCC
TGTCACTAACTACAGGCATTTCCCTGCCCTACATTGTTTTTGATTCTGCAAATTAATCATTAGGAAGAACCTGCTGATCATTGATA
TATCTTCGTAAATAATTTCAGATATGAAGTAACTGGGTTGACTGAAGAGGATATAAAAAATGCCATGCCTCTTAAAGAAGTGAAG
GATAAAATTTTGGAAATTTTGAACAATGGAGAGTCTACCGGGAGGCTTATGTTGGATGATGGAAAGGCCAGACTTCTTGTGGGT
CATGGTCTAGAGCATGATTTGGATAGCTTGAGGATGAATTACCCTGATCACATGCTGAGGTAATATAAATATTTTAAAGATTTTTG
ACCTGTCTAATTGATTTAAGTTCACTTTCATTGAAAGATACATGATAAAAAGGATGGATTGTTATTTGAATCGGACAAATGTCATA
ATATGGTTCAAATCACCCACTCAGCTGGTTATCTATGTTCAATTCAAAGTAAAATGTGTTTATAGATCTTCTCAATCTGCAATACA
ATCAAACTTGATTGTGATTTGGGATCTGGAAATGAGAATCACACTTCTTTTAAAACAATCTTGTGATGTATCTATGATTACCTCCTT
TACCTTATTTCCTGGGCTTAAACTTCAAGTCTTCTCCAGCAAAGGCCACCAAACTCGAGCAGCCACAGTTATGGTAAAAGTTTTG
ATGAAATTTCTACTAGTAGCATTTAAACTTAGCTTCAAAAGTTTTACTTGGAGAAAAATAACTCTGAACTCTGTTTGTATCTAGTTG
CATTGGTTTCATAAGTTTCACTTGGTGAAAAAATAAACCTGAACTCTGTTTGTATCTGGTTTCATGTGTTTTGGCTCTCACAGTTTT
GATTTCAATATTTTAAGACATTTTCGATTATCATATATTTTGTCTATGCAGCTGTATTGTTGCATTGAAGCATGGCCTTTTTTTTTTG
CGCCTTAAAATAACCTTTTCTTTGTGTAGGGATACTGCAAAATATCGTCCATTGATGAAGACAAATCTGGTTAGCCACTCGCTCA
AGTACCTCACCAGAACGTATCTAGGGCAAGTTATTTTTGCTTTCAGCACCCATCCTCATCACTAACACCCCCACCCCCTCTCCCT
TTAATTTTTTAAATTTTCTTTGTTATCCTGAAGTTTCTTGTTCATGTGGAGCTGCTATTGATATTTCTTTTCTTTTACAAGGGAAAAA
GGAAAGAGGAAACTTTTAACTGATTCTAACATTTCACTATTCCACTGTTAAACTATAAAGGTTCTTGGATGCTGTTTATGTTAGAA
AGTCTCGTAACTTTTTTAAAATTTATAGTTGATGTGATAGGGGAAGCAAGAATTCCTAAAGAAGGTCAAAGAAGAAATTTAGTAAA
ATGAAAGGAAAACAGTCTTTTGTTCCCATGCAATATCTATATGCATTTTATGATTTCAAATAACACGTCGGCAGAGTATGGTATGG
TCGGCCACTTGGTGTCTATTCAATGTTTGGAGTTCTGCGATCACTAATGTAATCTGTAAGAAGGATAGAAAATCACCGTATTTATT
TGCTTCACAGGTATGATATACAGTCTGGGGTGCATGATCCATACGAAGATTGTGTATCTGTCATGAGACTGTACAAGAGATTCC
GCAGACAAGATCATCAGGTAGAAGAAATTGGAAACCAAAATACTACCGGCAGTTTTGATTCTTACAAATACAAGGAGCTTGAGAA
GATGAGCCCAAATGAACTGTACCAGATATCAAAATCAGACTATAGATGCTGGTGCTTGGATTAGTATGGAGCCTTAGCTTGTGTA
CCTCTGCTTTCTTCATCTCTATGTATATAGCAATTTTTAT
SEQIDNO: 331
MDYRVESSETLRNKCAACYRQFNKIEHLVEHMRTSYHSAHEPTCGICKKHCRSFESLREHLIGPLPKQECRNIFSTRGCKFCLAILDS
PYALRVHQDRCQLSGVNAGLLGRFANLGIRDNTENGSTRGTQVVALACKMVGGGSDGSLDLCAKVCLIDEYENIIFQSYVKPLLPVTN
YRYETTGIRPEYLRDAMPLRQVQKKIQDFLCNGEPMWKIRPRGGKARILVGHGLDHDLDSLQVEYPQVMIRDTAKYPPLMKTSKLSN
SLKYLTQAYLGYDIQTGIQDPYEDCVATMKLYMRMRSQVHKTEAYPLASDPQNRNNFASWRQNELERMNPEQMLEASRSDYYCWC
LDSRDM
SEQIDNO: 332
AAAATCCAAAACAAAGGGATAACAATATTAATTGCTTTATTTTTCTTCAAGGAATCACGTATCATCAATCTCTCTCTAGATGCTTAC
ACTCCCACTCAGAAGCTATTCTCTTCCTCCTATCGTGGACTGCTATAAATACCTTCCATTCCCTCCACCTTTCCCTTCAAGTCTTG
TTCAGCTTCCTTAATTAATTACCTTTTTGTTTGAGTATACTAGTCTTTCAACCATCATGGACTACAGAGTTGAGTCTTCAGAAACTC
TGAGGAACAAGTGTGCGGCATGCTACAGACAATTCAACAAAATCGAGCACTTAGTGGAGCACATGAGAACCTCATATCACTCAG
CTCATGAACCCACATGTGGAATCTGTAAGAAACATTGCAGATCCTTTGAATCTCTCAGGGAACATCTTATAGGGCCATTGCCAAA
ACAAGAATGCAGGAATATATTCAGCACCAGAGGATGCAAATTCTGCTTAGCTATCCTTGACAGCCCTTATGCTCTCAGGGTTCA
CCAGGACAGATGCCAACTCTCTGGTGTAAATGCTGGCCTACTTGGTCGCTTTGCTAATTTGGGCATTCGTGACAATACTGAAAA
TGGTAGCACAAGAGGCACCCAAGTTGTTGCACTTGCTTGCAAAATGGTTGGTGGGGGCAGTGACGGCTCCCTGGATCTCTGTG
CAAAGGTCTGCCTCATCGACGAATACGAAAACATCATCTTCCAGTCTTATGTCAAGCCACTACTTCCAGTCACAAACTATAGGTA
TGAAACAACTGGCATTCGTCCTGAATACTTAAGGGATGCAATGCCTCTGAGGCAAGTGCAAAAGAAGATTCAAGACTTCCTCTG
CAATGGGGAACCAATGTGGAAGATTCGACCTAGAGGCGGAAAAGCTAGGATTCTTGTGGGCCATGGTTTGGATCATGATCTTG
ACTCTTTGCAGGTGGAATATCCACAAGTAATGATCAGGGATACTGCAAAATACCCTCCATTGATGAAAACAAGCAAGCTCAGCA
ACTCACTCAAGTATCTAACACAAGCATATCTTGGGTATGACATTCAAACTGGCATTCAAGACCCTTATGAGGATTGTGTTGCAAC
AATGAAGCTCTACATGAGAATGCGATCCCAAGTTCACAAGACAGAGGCTTACCCACTAGCTTCTGACCCACAGAACAGGAACAA
CTTTGCATCGTGGCGCCAAAATGAGCTCGAGAGGATGAACCCAGAACAAATGCTGGAAGCCTCAAGGTCTGATTACTATTGTTG
GTGTCTGGACTCCAGGGATATGTAGTGCCTGAAAGTGAAAGGAGGCTATATGAAAATAATA
SEQIDNO: 333
AAAATCCAAAACAAAGGGATAACAATATTAATTGCTTTATTTTTCTTCAAGGAATCACGTATCATCAATCTCTCTCTAGATGCTTAC
ACTCCCACTCAGAAGCTATTCTCTTCCTCCTATCGTGGACTGCTATAAATACCTTCCATTCCCTCCACCTTTCCCTTCAAGTCTTG
TTCAGCTTCCTTAATTAATTACCTTTTTGTTTGAGTATACTAGTCTTTCAACCATCATGGACTACAGAGTTGAGTCTTCAGAAACTC
TGAGGTACTTGACTCTCTCTCTCTCTCTCTCTCTCTCTCTCTCTCTCTCTCTCTCTCTCTCTCTCTCTCTCTCTCTCTCTCTCTCTC
TCCCCCCATCAGAATATCAATCTTCTATGCATCATGCATGATCCTAGTTGTTTAACTAATCACAATATATACATGTTTATGTGACTT
TTAATGATCAGGAACAAGTGTGCGGCATGCTACAGACAATTCAACAAAATCGAGCACTTAGTGGAGCACATGAGAACCTCATAT
CACTCAGCTCATGAACCCACATGTGGAATCTGTAAGAAACATTGCAGATCCTTTGAATCTCTCAGGGAACATCTTATAGGTAATC
AAACCAATTAATCAATTAATTCTACTCTTGCACGTACACACACACATATAAAATATATATTATATTCTTTAATACTTCCAGAATTCTT
TTAATTTCCAGTTAATAGTATGTATGTATTGGTTTTTGTTCAGGGCCATTGCCAAAACAAGAATGCAGGAATATATTCAGCACCAG
AGGATGCAAATTCTGCTTAGCTATCCTTGACAGCCCTTATGCTCTCAGGGTTCACCAGGACAGATGCCAACTCTCTGGTGTAAA
TGCTGTAATAATCCTAAATTAACACACCACCTTAATTATGAATTTTCTTTCTTTCTTTCTTTCTTTCAATGCAAATTAGCTCAATTCT
TAATATTATTCATACATGCAGGGCCTACTTGGTCGCTTTGCTAATTTGGGCATTCGTGACAATACTGAAAATGGTAGCACAAGAG
GCACCCAAGTTGTTGCACTTGCTTGCAAAATGGTTGGTGGGGGCAGTGACGGCTCCCTGGATCTCTGTGCAAAGGTCTGCCTC
ATCGACGAATACGAAAACATCATCTTCCAGTCTTATGTCAAGCCACTACTTCCAGTCACAAACTATAGGTAGCTATATTATATGGA
AAAGATGATATGTGTAGGTAGAGAGCTTCTTCTAGTATATACTAACTATAGTACAACATATATACTTGTGTATATGCATATAGGTA
TGAAACAACTGGCATTCGTCCTGAATACTTAAGGGATGCAATGCCTCTGAGGCAAGTGCAAAAGAAGATTCAAGACTTCCTCTG
CAATGGGGAACCAATGTGGAAGATTCGACCTAGAGGCGGAAAAGCTAGGATTCTTGTGGGCCATGGTTTGGATCATGATCTTG
ACTCTTTGCAGGTGGAATATCCACAAGTAATGATCAGGTAATTTTCATTTTCTTCTACCCTACCAAACTTAATTGCAAGATAAGCA
CAAAATTACTTGTGTTACTAATCATCCCATATGTTGTCTTAGTTCAATTCCCCATTATTTCCCCATTTTCCTTTGTTCAATCCAGCA
TATATAATTTTAGATCAAGCTCAAAACTTGAGAAACATCATATATGTAGAACTTTCCATTAGTAATTTCAAATGTATGTGACTATAT
GTAGGGATACTGCAAAATACCCTCCATTGATGAAAACAAGCAAGCTCAGCAACTCACTCAAGTATCTAACACAAGCATATCTTGG
GCAAGTACCTTATTATATCAATTTCGTCTATCTCAAATTCCAAATATAAACCTAGCTAGCTATGTGAGTACTAATGGAACTCCTAC
ATATTTACAGGTATGACATTCAAACTGGCATTCAAGACCCTTATGAGGATTGTGTTGCAACAATGAAGCTCTACATGAGAATGCG
ATCCCAAGTTCACAAGACAGAGGCTTACCCACTAGCTTCTGACCCACAGAACAGGAACAACTTTGCATCGTGGCGCCAAAATGA
GCTCGAGAGGATGAACCCAGAACAAATGCTGGAAGCCTCAAGGTCTGATTACTATTGTTGGTGTCTGGACTCCAGGGATATGTA
GTGCCTGAAAGTGAAAGGAGGCTATATGAAAATAATA
SEQIDNO: 334
MDFEPELPKTTNTRYKCAACYKQYKKKEHLVDHINVSFHSVHQPRCGVCKKHFKSFESQREHVTGPLAKENCKTVFSEQGCDLCLKI
FDSPNSLSAHKYMCRLSAPVPLVTMMERCEEFKHDINSSNEYHVGDGTEAIALDCEMVAGGSDGSLDLCARVCLIDEDETILFHTYVQ
PQYPVVNYRYEVTGLTEEHLRDGMPLQKVQDKVLQILYNGKTKLLVGHSVENDLDCLRMNYPDYLLRDTANYHPLMKTNLVSHPLKY
LTRTYLGYDIQSGFHDPYEDCVSAMRLYKRFCALDHQKEGNVASLAMLCAKDIPGSFDSWETDKLEKMTLDELYEMSRPNYKCWCL
DSVQAMQPQH
SEQIDNO: 335
ATTTATATTACTTTATTAGTGATTTTGATTCACTATCTAATATCTATGAAGAGAAATTACTCATAACATAATATTGATATAAAGATTC
GTATATATAAGAGTGATTTCTTGCCAAATATAATCAATTATGATAAGAAAAAAAATGACGGTCGCATAGAGAAATTAATGGTACAA
TTACTTGAAAAACCCTAATTCTCTGACTCTCTGTGAGAGAAGTCAGAAAAAATATCCCCCATTTGGGCTCACCTCCATTTTGTGC
ACATTTCTCGTACTGCTGTTTTCCCGCCAATTTTGAAGCCTCGTCGACGGCGTTGCTGAGAGCAGCTCTGGGCTCCATGGACTT
CGAACCAGAACTTCCAAAAACCACAAACACAAGGTACAAATGTGCTGCATGCTACAAGCAGTATAAGAAAAAGGAACATCTAGT
CGATCACATTAATGTCTCGTTCCATTCAGTTCATCAGCCTAGATGTGGAGTGTGTAAAAAGCACTTCAAATCTTTTGAATCACAG
AGGGAGCATGTCACCGGACCACTGGCAAAAGAAAATTGCAAGACGGTTTTCTCTGAACAAGGTTGTGATCTATGTTTGAAGATT
TTTGATAGCCCTAATTCTCTGAGCGCTCATAAATATATGTGTCGCCTATCTGCACCTGTTCCTCTGGTAACAATGATGGAGCGAT
GTGAAGAATTTAAACATGATATAAACTCCAGCAACGAATATCACGTTGGAGATGGTACGGAAGCAATTGCTCTGGATTGTGAAAT
GGTTGCTGGTGGAAGTGATGGATCACTGGACCTGTGTGCAAGGGTGTGCCTGATTGATGAAGACGAGACTATCCTCTTCCACA
CTTATGTGCAGCCCCAATATCCTGTCGTTAATTACAGATATGAAGTTACTGGGTTAACAGAAGAGCATCTCAGAGATGGCATGC
CACTCCAAAAAGTGCAAGATAAGGTTTTGCAAATTTTGTATAATGGAAAGACCAAGCTTCTTGTGGGGCATAGTGTAGAGAATGA
TTTGGATTGCTTGAGAATGAACTATCCTGATTACCTGCTGAGGGATACTGCAAACTACCATCCTCTGATGAAGACAAATCTGGTT
AGCCACCCGCTCAAGTACCTCACCAGAACATATCTAGGGTATGATATCCAGTCAGGTTTTCATGATCCATATGAAGATTGTGTGT
CTGCTATGAGACTCTACAAAAGGTTCTGTGCGCTAGATCATCAGAAAGAAGGCAATGTGGCTTCACTTGCTATGCTGTGTGCAA
AAGACATCCCTGGAAGTTTTGATTCTTGGGAAACCGACAAACTTGAGAAGATGACACTCGATGAGCTTTATGAAATGTCGAGAC
CAAACTACAAATGTTGGTGCCTGGATTCAGTGCAAGCAATGCAACCTCAGCATTAGATTCTCACTTGAAGTTAAGTCATTGCAAT
TTTCGTTCACATACCGCAGGATTGACAGAAACATCACATGTAGGAATATATGTAGGAATTACAAGTCTGAAGCTGTTGGAGATTC
TGAGCAAGGGTACCATCGCACATGTAGTAGGAAAATCTTTTTAGGGGGTCATGCCTTAATGTTATATAAGTTTGTTCATGCCTCC
TTTTTCGTCATCGGTCTCGGATGGTGAAATGATGCAAATTCTCTAAGGCCCTGTTTGGTTTGGGAGAGAGGATTTGCATTTGCAT
TTAGATTTCAAATCTATGGATATAATTTCATGACATGATTTTGCTGCTTGGGTTTTGGGGTAGATTTTATTTGTGTGATTCCCTAAA
GAGTACAGAGCTTCTGCTCTGGTGTGGCGTGGTGCACGTGATGAGATGCTTAAAGGTTCAGTGGGTGAGTTAGTAATTTATAAG
GTGGGTATACGTATATATTTCTCTAGAAGCAGAACTACTTTTTGTTTTATACGTATAAACCGAAAAAGGAGGGTTGGGCCTCTGT
CACTATTTACAGCCCAACTAGCCCATCTTCAGCTGTCAGATTAGCAACGACTTGTGGGCTTGTAGCATCCTAAGGTAATAGTAAT
ACCAAAGCAAATGTAAATTCTATTCCGAAAAAAGTTAACCCAAAAAGAAAAAAAATCAGAGGACTACTGTTCAAC
SEQIDNO: 336
TTTATATTACTTTATTAGTGATTTTGATTCACTATCTAATATCTATGAAGAGAAATTACTCATAACATAATATTGATATAAAGATTCG
TATATATAAGAGTGATTTCTTGCCAAATATAATCAATTATGATAAGAAAAAAAATGACGGTCGCATAGAGAAATTAATGGTACAAT
TACTTGAAAAACCCTAATTCTCTGACTCTCTGTGAGAGAAGTCAGAAAAAATATCCCCCATTTGGGCTCACCTCCATTTTGTGCA
CATTTCTCGTACTGCTGTTTTCCCGCCAATTTTGAAGCCTCGTCGACGGCGTTGCTGAGAGCAGCTCTGGGCTCCATGGACTTC
GAACCAGAACTTCCAAAAACCACAAACACAAGGTTCCATCTCTCTCTCTCTCTCTCTCTCTCTCTCACATCTTCTGCAATTTGTGA
GGTTTATGGCTTTTATTTCCACATTTCCACAAGGACTTGCATGCTTGCTGAGAAATTGAATACAGATTTTGTTTGGGTGGCTTATT
TATGAACTTTTCTGATAAGCTAATTGAGGTGCATTGCAGAATTTGACATGGTTTATCACGCATTGTTTGATTCTAGTTTTTTCGAA
GTGTATTTCTTGATTTGGAAATAAATTTCCTTTGATTTTGGAAACAAATCCTTCCTTGGGGATCATTTCACTTAATTAATCCCAATT
GTCCTGCTATAGTAATGTATCTAAACTTGAATATTTAAGAACAATTGCAAAACTAGTTGACAAACTGTGCTATTAGGTATATACAC
AATGTGATATGTGATTTTTTTGTTGTTGAATATTTTGAAAAGATTCTTGACCTGTGGGCTTTGCAGGTACAAATGTGCTGCATGCT
ACAAGCAGTATAAGAAAAAGGAACATCTAGTCGATCACATTAATGTCTCGTTCCATTCAGTTCATCAGCCTAGATGTGGAGTGTG
TAAAAAGCACTTCAAATCTTTTGAATCACAGAGGGAGCATGTCACCGGTGAGATGTAATTCCGAGTTGCATTGTAATTGATCAGA
TCATTGCATAGTCGTTTTGATTCATCTATAAGATTGCTTGCTGTTTGATATCCTTTCTCTGGTTGCTATGATTTAGGACCACTGGC
AAAAGAAAATTGCAAGACGGTTTTCTCTGAACAAGGTTGTGATCTATGTTTGAAGATTTTTGATAGCCCTAATTCTCTGAGCGCT
CATAAATATATGTGTCGCCTATCTGCACCTGTTCCTCTGGTAAGTTGATTAGTTGAACATCAAGCGTCCTGTATGGAATTGGGAA
ACTTGTCAGCTTCCAGTATTCTGAATCTTTTACCCTTTTTACCCATTAAAGATACTCTTAGCAGCAACCGCATATAAGATATGCAC
ATACATTACTTATATTTTCAGTCAAAGTGTTGCAGGTAACAATGATGGAGCGATGTGAAGAATTTAAACATGATATAAACTCCAGC
AACGAATATCACGTTGGAGATGGTACGGAAGCAATTGCTCTGGATTGTGAAATGGTTGCTGGTGGAAGTGATGGATCACTGGA
CCTGTGTGCAAGGGTGTGCCTGATTGATGAAGACGAGACTATCCTCTTCCACACTTATGTGCAGCCCCAATATCCTGTCGTTAA
TTACAGGTACTAGTGTGTTATGTTTCTCATCTTTGATTTCTTACAAGAATTTTGCGAATAGGCCTCTGATTATTCCCACGTGTTGA
CAATGTTTCCATTCCAGATATGAAGTTACTGGGTTAACAGAAGAGCATCTCAGAGATGGCATGCCACTCCAAAAAGTGCAAGAT
AAGGTTTTGCAAATTTTGTATAATGGAAAGACCAAGCTTCTTGTGGGGCATAGTGTAGAGAATGATTTGGATTGCTTGAGAATGA
ACTATCCTGATTACCTGCTGAGGTATAAGAGTTTCTGTTTTCTTCTGTAATCCTCTTCTTGAGATATGTGAATCACGAGCACAGTG
TTTCTATAGTTTTTCTAGATCTGATTGAACTATCATGTCATGCTATGTTCTGGTTCTCATATGCACTTGTATTCTTCTCTTCAGTCC
CTTTCTTTATTATATAGGCCATAGTGTTTGGGCTGCTTTGTGTAGATGTAAGATAGACGGTGTCACTGTTTCTTGGACAATTGCGT
TTATGGCTGTACTTCCTTTCATTCAGGCCAGTCAAGATTATGGCCTTAGTCATGATGAAATTTTGCATAAAGGACCAGCTTATATG
GCATCTATTTAACATAGATTGAGCAAAGAAGCCTGTTCTCTGTTGCCTCTTAGATGCTTGATGGTTACTTATTTTATATGCAGGGA
TACTGCAAACTACCATCCTCTGATGAAGACAAATCTGGTTAGCCACCCGCTCAAGTACCTCACCAGAACATATCTAGGGCAAGT
TATTCTTCTTCTCAGATTTTTACTTTTTTAGTCTCTATGTTATTTGCTTGTAGAAGCAGGCTCTAGTTTTAATTGCTCCATTGAATAT
TTAGTGTCATGAGGCTTTGGTTTTACAGGTATGATATCCAGTCAGGTTTTCATGATCCATATGAAGATTGTGTGTCTGCTATGAG
ACTCTACAAAAGGTTCTGTGCGCTAGATCATCAGAAAGAAGGCAATGTGGCTTCACTTGCTATGCTGTGTGCAAAAGACATCCC
TGGAAGTTTTGATTCTTGGGAAACCGACAAACTTGAGAAGATGACACTCGATGAGCTTTATGAAATGTCGAGACCAAACTACAAA
TGTTGGTGCCTGGATTCAGTGCAAGCAATGCAACCTCAGCATTAGATTCTCACTTGAAGTTAAGTCATTGCAATTTTCGTTCACA
TACCGCAGGATTGACAGAAACATCACATGTAGGAATATATGTAGGAATTACAAGTCTGAAGCTGTTGGAGATTCTGAGCAAGGG
TACCATCGCACATGTAGTAGGAAAATCTTTTTAGGGGGTCATGCCTTAATGTTATATAAGTTTGTTCATGCCTCCTTTTTCGTCAT
CGGTCTCGGATGGTGAAATGATGCAAATTCTCTAAGGCCCTGTTTGGTTTGGGAGAGAGGATTTGCATTTGCATTTAGATTTCAA
ATCTATGGATATAATTTCATGACATGATTTTGCTGCTTGGGTTTTGGGGTAGATTTTATTTGTGTGATTCCCTAAAGAGTACAGAG
CTTCTGCTCTGGTGTGGCGTGGTGCACGTGATGAGATGCTTAAAGGTTCAGTGGGTGAGTTAGTAATTTATAAGGTGGGTATAC
GTATATATTTCTCTAGAAGCAGAACTACTTTTTGTTTTATACGTATAAACCGAAAAAGGAGGGTTGGGCCTCTGTCACTATTTACA
GCCCAACTAGCCCATCTTCAGCTGTCAGATTAGCAACGACTTGTGGGCTTGTAGCATCCTAAGGTAATAGTAATACCAAAGCAA
ATGTAAATTCTATTCCGAAAAAAGTTAACCCAAAAAGAAAAAAAATCAGAGGACTACTGTTCAAC
SEQIDNO: 337
MRTSYHSVHEPTCGICKKHCRSFESLREHLIGPLPKVECARIFSVRGCNLCLDILGSPNALRAHRGTCQLSRGNTGALLSRMANLGIQ
DDLNSRTRGSKVVALGCKMVGGGTDGSLDLCARVCLIDEYENIIFHTYVKPQIPVTNYRYETTGTRPEFLRDAMPVKQVQRKIQDFLC
NGEPIWKIRSRGGKARILVGHGLDHDLDCLQMEYPTLMIRDTAKYPPLMKTSKLSNSLKYLTQAYLGYDIQTGIQDPYEDCVATMRLY
MRMRSQSHKIEDYPLASDPQNRNNFASGRQSELERMTPDEMLEISRSDYYCWCLDTKEGS
SEQIDNO: 338
GAAGTTTCTCTATGATTAATCATCACGGCCAGGAACAAGTGTGCAGCTTGCTACAGACAATTCAACAGGTTGGAGCACCTGGTT
GATCACATGAGGACCTCGTATCACTCGGTTCATGAGCCAACCTGTGGAATTTGTAAGAAACACTGCAGATCTTTTGAATCTCTCA
GGGAGCATCTCATAGGGCCGTTGCCAAAAGTGGAATGCGCAAGAATATTCAGTGTCCGCGGATGCAACCTCTGCTTAGATATC
CTTGGCAGCCCTAATGCACTTAGGGCTCATCGAGGGACATGCCAACTTTCACGCGGAAACACCGGAGCGCTGCTTTCTCGCAT
GGCTAACCTAGGTATTCAGGATGACCTGAATAGCAGAACAAGAGGCTCAAAGGTGGTTGCACTGGGCTGCAAAATGGTAGGGG
GTGGAACCGATGGCTCGCTGGATCTTTGCGCAAGGGTTTGCCTCATCGATGAATATGAAAACATCATCTTTCATACTTATGTCAA
GCCCCAAATTCCAGTCACAAACTACAGGTATGAAACCACAGGGACAAGGCCCGAATTCTTGAGGGATGCAATGCCAGTGAAGC
AAGTCCAAAGAAAAATCCAAGACTTCCTTTGCAATGGAGAGCCCATATGGAAGATTCGTTCTAGAGGTGGAAAAGCTAGGATTC
TCGTGGGTCATGGTTTGGATCATGACCTGGACTGTCTACAAATGGAGTACCCAACGCTAATGATCAGGGACACAGCTAAGTATC
CTCCATTGATGAAAACAAGCAAGCTCAGCAACTCACTGAAGTATCTGACACAAGCATACCTCGGGTATGACATCCAAACTGGCA
TACAAGATCCTTATGAGGATTGTGTTGCAACAATGAGGCTTTACATGAGAATGAGATCCCAAAGTCATAAAATAGAGGACTACCC
GCTCGCGTCTGACCCGCAGAACCGAAACAACTTTGCATCAGGGAGGCAAAGTGAGCTAGAGAGGATGACCCCAGATGAAATGT
TGGAAATCTCCAGGTCTGATTACTATTGTTGGTGCCTGGACACGAAGGAAGGCTCCTGA
SEQIDNO: 339
GAAGTTTCTCTATGATTAATCATCACGGCCAGGAACAAGTGTGCAGCTTGCTACAGACAATTCAACAGGTTGGAGCACCTGGTT
GATCACATGAGGACCTCGTATCACTCGGTTCATGAGCCAACCTGTGGAATTTGTAAGAAACACTGCAGATCTTTTGAATCTCTCA
GGGAGCATCTCATAGGTAAAATCCAAGACAACTACATGAAACCCGAACCATATTGCCCTTATGAAGATATATCATTCAAAGGAAG
TACTTCTTGTCTTGGGCAGGGCCGTTGCCAAAAGTGGAATGCGCAAGAATATTCAGTGTCCGCGGATGCAACCTCTGCTTAGAT
ATCCTTGGCAGCCCTAATGCACTTAGGGCTCATCGAGGGACATGCCAACTTTCACGCGGAAACACCGTATAAATCTTCAGCTTG
CATGACAATACAATTTGAAGTTCTAATGACTTTGTCTAGTTTCAAGCATGTCACATGTTACTGCCCTTTTCTTTTCCAGGGAGCGC
TGCTTTCTCGCATGGCTAACCTAGGTATTCAGGATGACCTGAATAGCAGAACAAGAGGCTCAAAGGTGGTTGCACTGGGCTGC
AAAATGGTAGGGGGTGGAACCGATGGCTCGCTGGATCTTTGCGCAAGGGTTTGCCTCATCGATGAATATGAAAACATCATCTTT
CATACTTATGTCAAGCCCCAAATTCCAGTCACAAACTACAGGTTTGATGAGTCTCTTTAAAGCTAACTGTATTTCCACCCTGGCT
GAATTTCAATTCTAAATATAAATTCTTGAAAACATTGTAGGTATGAAACCACAGGGACAAGGCCCGAATTCTTGAGGGATGCAAT
GCCAGTGAAGCAAGTCCAAAGAAAAATCCAAGACTTCCTTTGCAATGGAGAGCCCATATGGAAGATTCGTTCTAGAGGTGGAAA
AGCTAGGATTCTCGTGGGTCATGGTTTGGATCATGACCTGGACTGTCTACAAATGGAGTACCCAACGCTAATGATCAGGTAAAA
AGCCCTTCAAATTAAGAAAATCAAAATTGCACCATAGCTTTGAAACTATAACAGTGGGTCTCAAACTAGAATCAACCAGCATCATT
CATAATTCCATCATGGCTATTATACTCATGTAACAGAAAATCAAGGTATTTTCTGAACAGGTGATTGGTGTCCAGTTCTCTATTCT
CAACAAAAACTCTTAAGGGTGGAGCCTCAAAAAAATAAATAAATAAATAAAAACAATTGCAAAATCAGATGTAATATAATACTGAA
TTAATCTGAAATGCATATTGGATCAGGGACACAGCTAAGTATCCTCCATTGATGAAAACAAGCAAGCTCAGCAACTCACTGAAGT
ATCTGACACAAGCATACCTCGGGTAAGTACATCACATGCATTTCAATTTCGAGTTCACTGCCAAAATTTAAATATATATTTCAGGA
AAGGAATGAAACTTCTGGTTTCCAGGTATGACATCCAAACTGGCATACAAGATCCTTATGAGGATTGTGTTGCAACAATGAGGCT
TTACATGAGAATGAGATCCCAAAGTCATAAAATAGAGGACTACCCGCTCGCGTCTGACCCGCAGAACCGAAACAACTTTGCATC
AGGGAGGCAAAGTGAGCTAGAGAGGATGACCCCAGATGAAATGTTGGAAATCTCCAGGTCTGATTACTATTGTTGGTGCCTGG
ACACGAAGGAAGGCTCCTGA
SEQIDNO: 340
MGSEAVPSESRTTRYKCLACFRQYKKKEHLVEHMRVSYHSVHQPKCGVCQKQFKSFQSLREHLTGNLPKKNCLKIFSEQGCNYCLK
IFKNADSLSEHKEMCQLPVPVTIEKIRMPSTESQIDFSDSINRDHTSRGLEAIAIDCEKVGGGSDGSLDLCARVCLIDEDENIIFHTYVQP
QIPVTNYRYELTGITEAHLRDAMPLKEVREKVMEVLRNGESIGRLRLDGGKGRLLVGHDLRHDLDCLRMHYPDHLLRDTAKYLPLMK
TNLVSFSLKSLTQKYLGYDIQSGMHDPYEDCVSVMRLYKRMQSQDHSDEGIGTAIHYRLRVMIFGHNRTDTGGSMRT
SEQIDNO: 341
CTGATTTTGTTTTTTTTTGGGTTCGGGTTGTTAGTGAGGTTTCCAGAGAATCCATGGGCTCGGAGGCAGTTCCTTCGGAATCTAG
AACTACAAGGTACAAATGTTTGGCCTGCTTCAGGCAATATAAGAAGAAGGAGCATCTAGTTGAGCACATGAGGGTATCATATCA
TTCTGTGCATCAGCCAAAATGTGGGGTCTGCCAGAAGCAGTTCAAATCTTTCCAGTCACTAAGGGAACACCTTACTGGTAATTT
GCCCAAGAAAAATTGTTTGAAGATATTCTCTGAGCAAGGTTGCAACTACTGTTTGAAAATCTTCAAGAATGCTGATTCTCTCAGT
GAGCATAAAGAAATGTGTCAATTACCTGTGCCTGTTACAATAGAAAAAATCAGGATGCCATCTACAGAATCTCAAATTGATTTTTC
AGATTCTATTAATAGAGACCACACTAGCAGAGGTCTTGAAGCAATTGCCATTGACTGTGAAAAGGTTGGTGGCGGAAGTGATGG
ATCGCTTGACCTTTGTGCTAGGGTATGCCTCATAGATGAAGATGAGAATATAATTTTTCACACTTATGTGCAGCCACAAATTCCT
GTCACCAATTACAGATATGAGTTAACTGGGATTACTGAAGCACATCTGAGAGATGCCATGCCACTAAAGGAAGTGCGGGAAAAA
GTTATGGAAGTTTTACGAAATGGAGAATCCATTGGGCGACTGCGTTTGGATGGTGGGAAGGGCAGACTTCTTGTGGGTCATGA
CCTACGCCATGACCTGGATTGCTTGAGAATGCACTATCCTGATCACTTGCTGAGGGATACTGCAAAATACCTCCCCTTGATGAA
AACAAATTTAGTCAGCTTCTCCCTGAAGTCGCTCACTCAAAAATATCTTGGGTACGATATCCAGTCAGGGATGCATGATCCATAT
GAAGATTGTGTATCTGTAATGAGGCTGTACAAGAGAATGCAATCTCAAGATCACTCAGACGAAGGAATTGGAACAGCCATTCAT
TACAGGCTGAGAGTAATGATCTTTGGGCACAATAGAACTGATACAGGGGGTTCGATGAGAACATGACAGGAAAAGAAGCGAAT
ATCTCCGGTGAGTCTACAACATTGTAGCCATGAAATGAATGTGCTACAATGTTGAAGATATTTCGTCGAATATGAGATCATTATG
AGAACATTTCCTGATATAGCAAACTCAATATTGATAGGGCTTCTTCAATACCCAACCCTGTTTTCCTATGCAAACATTAAACAGAT
CTTGTGCTTGTGTGTGTGTGTGTGTGGGTTTGATTGTTTTGTTGGAGTTGCCTTTTTATTATTTTTTATTTTTTAAATATTTTTATTT
TGCAAATACTTTCCCATGTAGTGAAGAATGAGGAAGAGATCTTATATAATTTCGCATGTAAGGGGTTA
SEQIDNO: 342
CTGATTTTGTTTTTTTTTGGGTTCGGGTTGTTAGTGAGGTTTCCAGAGAATCCATGGGCTCGGAGGCAGTTCCTTCGGAATCTAG
AACTACAAGGTTTGTTTTTCTGTTTTTTTTTTTTAATCGATATTCAGTGGTGGGTTTATTTGGATGATGAGAGAATGCAAGGAAAAA
GAGAGAAATGACGGTTTTGAAGGAAATGCTATTGCCATTCTTGGTTTTACCGCTTCCACGGACCTCAACGTGCGAGTGTGGAAT
TGGGTTCTCTGCATTTGCCGTGGAAGCAACGGGCGAATTTGGCAATAGCATTTCCGGTTTTCGTTTGAACTTGAAGACCCACTT
CAGTGAGCATCGTCCATTAAAATCAGTGGTTACGACGATTATCTTAGAATTTGGATGCTTTTAGGTTTATTAGTTTCACGCTTAGT
TCTCTTTCCTTTTGGTTTGATTCACTTCTGCTCTGCCCTTTCGTTTAATTTACCAACTGAAGATTTGATAAGAAGGGAATTATTTGA
ATTTGATTTGATTAAATTATCGTTTTTGATCTGAAAATTGGCATTTTAACCTATAGTTGGTCATTAGATTCTATTGACCATGTAGGG
CTGATTGGACTTGATTAAATTATTGCTTTTGACCTGTAATTGTTGTTTTTGATCGGCAATTGACTATTAGCCTCTTTTGATCAATAG
GGGCGATATGATTTTAGCTTATTTGGAATTCCAGTGGTCGGTCCATGTTGCACTATATGCTTGAATTCAGTTTGTTTATTTTCCTG
TTGTCTGAATTTATTTTCCTTGTTGAATATTCTTAAATTCAGATTATTCCTTGAGTTGTTAGTAACTTAGTGTGTATAGGTGAATAAT
GGTCCGTGCTAGTGCTCCAATGGTAAAAGTAGTTTTACTTCTTTTAGCGTGTTGGTCAAGGTTCTTAATAATTGGTATCAGAGCT
AGAATCCACCTCCCAAATTTAGGTCATGGAAATGGCTACAATTTATTTATGCTATACCTAGGAATGTGTTTGATTTGACTGGTTCC
ATAACTCCTCAGTTTTTCCCATGACTAGTTTTATTTTAATAATTTCCCCATTATCATTATTCCAATCACAAAGCAGTGAATTAAGAG
CTACAACAATAATTACAACTTAAACGAAACAAGTCCATGTTTCCGAGGCAAAACAAAAAGGTTGCTTTGTATGTTTCTATATTAAC
ACATTGGGCTATTCTAGAAGAAACCTCTTGAAGGCAAAAGTGAAGGGAACTTGAAGAGGGGGATAAAAGTATTAAGTTCTTCCA
CAAGATGAAAAATGCTCATAAAAGATGTAATCTCATTGGGAGGTTGAAGGTTAATGGGGCTTGGCTAATAGAGGATAAAGAGAG
TTCACCAGGAGTGATGAAGGAGTTCCATCTTTTATTGACTAATCCAAGGGATTGAAGACCTAGCATTGGTGTGTTGTCTTTTAAG
ACTTTGTGCAAAGACAACTCTCTAGCTGTGGAAGAGCCTTTTTTGAGGACTTTTGTGCTGCATTGTTAGGGATGGGAGAAGATA
AAGCCCTTGGTTCAAATGGTCTCTCGATGACATTTTGACAATATTATTAGAACTTTGTTAAAGGTTAAGTGTTAGGGCTATTCGTG
GAGTTCTTTGGGAAGGGGATGTTTGAGAAAAGTTCAAATGCTACTTTTTGGTGTTGGTTCTGAGAGACAAAAGAGTTGAAGGAC
TTCAGACCAAGAAGCCTAGTGGAGGGATTGTACAAGCTTCTTACCAAGGTTTTAGCTAATATGCTTAAGAAACCGGCATTTGCTT
CGTAAAATGCTTTTGTGGAGTGTAGGCATATTTTAGATGTGGCTCTCATGGCTAATGAAGCCATTGACTCAAGGAGGAAGAACT
CTAGTGGTGGTTAGTTTGTAAGTTAGATATTGAAAAAGCCTATTACTTGGTGAACTAGAGCTTTCTTTTGAACTACTAGGTAAAAT
TGGATTTAAGCAGAAGTAGGTTGAATGCATTCAGTTTTCTATCTCAACAAGGTTCTCTATGATGGTGAACAACACACTTTTCAGCT
TTTTTTGAGCTCAAGGAGCTTGAGTTAGGGTGACATCCTCTCTCCTTATTTGTTTGTAATGGTAATAGAACTCTTAGTGTTCTTTT
GTGTTAAGGGAGGGCGATTTTATTTCAAGCTCAAGGTAGGAGGTAGGGAATAAGAGAGTGTAGAGGTTTCCCACCTCCAATTTG
TTGATACTTTCCTTTGTGAGCCCTGTTTGGAACAGTTGACCTGCTTGAGTTGGATTCTGACTTGGCTTGAGATTTCCTCTAATAAA
AAATTTAACTTGGAGAAAGGTGAATTAATTCTTCTGAAAAATATGGAGACTCCAGCATCCTTGCTGTGTTATGGGGTTGGGAAGT
TGCCTACCAAGCATATTTAGGTCTACTTTTGTTGAGCCCCATTTATGTCATCAATGATTTGGGATGTAGTAGGGGAAAATTTTCAT
TAAAAGTTGTCAAGGTGGAAGCAAAAATAGCTCTTAGAGGGTAGGAGAAAAAAAGTTAAGTTCTAATAAGATGTTTGTTGTCATG
ACTCTCCTTTAAAGTGGCTTTCCTTTCTCTATATTCTTTTAGCGTTTCTAAGTCGCCGAGTTCCCGAATAGCAATATATGGGTGGA
TAGAGTTCCAACCAAAATTGCATTTTTTTGCATGGGAAGCCGCTTGGGGGAAGGTTCTCACTCTTGATAGGCTTCAGAGAAGAG
GGTGGCAGTTCCCTAACTGCTGTGTCTTGTGTGGTTGTGAAGAGGAAACGATAAATCATATTTTAATTCATTGTACAGTTGTCAG
AGTTTTGTGGGATATCATTCTTGGGTTGTTTGGTGTTTAGTGGGCCTTTCCAGAATCTGTAAAAGAGGTTTTATTTAGCTGGAAG
GGCTCTTTTGTGGGAAAAAAGAGGAAAAAACTGTGGAAGTCCATCCCGCTGTACATTTTTCAGACAATTTGGAAGGAGAGGAAT
AGACTATCTTTTAGGGAGAGGGGGGGGATGTTAGCAGTTCAAAAGTTAAAAACTTCATTTGTGTGTAATGTTTGGGGTTGGGCT
AAATTGTACATTGGTGTGGAGTTGATCTCCCTTATAGGCTTCTTGGAGTGTCTAGCCTCCACTTAGGGGTTGGGGTGTTTTGTTC
TGTTTTTGGTTGGGAGGCCTTAGTCGCCTCGTATACTTCCTGTATGCTTTGCGGCTATTTTGCCTCCTGATAATATATTCTGCGC
TTACTTATAAAAAAAAAAGGATACTTAAGTGGTCAATGTTTATAAGGCTTCTAGTGGGCAGGGCCATGGAACCTATGTTTCTTAA
GGCAGTTCCAAGATTATGAGGAAAGATTGGTTTGAAACAGTACCAAAGATGGGAAGTCCTCTATTAGATCCTTCTATTTTGCTTT
GGAGTTGAATGATAGAGAGGCTTTCCCTTTAAAGGAGGTCTAAAGCCCTTGGGTCCCCTTGAAGGTGAGCATTTTTGCTCTGGA
AAGGATTCTAACCTTAGATCTTCCTAAAAAAAAAGGTGACTTTCAAATTGTATTTGCATTTATAATAGAGAATTAAAATTGGCTAAT
CATACCCTCGTCTAATGTGGAATATTAAGAGGATTGTATCTTTTGTTTGATCTCTTTGACATCTCTTAGATTCTCACCAATTCAATG
GGAGAGATTTTGTAGGGCAAGCATGGAAGTTTTGTGGGGAAGAAGAAGAAACCAATGTTTTCGAACTTAGATTGGATTGATTGG
TCAGACCAATTCAACCTTTAATCAGACCACTTTTTGGTTCAAGTAAGATAATTGGCTCGTTTTTCTATTAAAATGGCATTGGACTG
GCCAACGGTCAAACAGCCAAACTGGACAGACTGACCATTTTTCTTTTGGCCCAAAAGGTTATTGGGCTGAAATAATTTTGGGTA
GGTGTCCTCTCTGGGTTACTCTCCAACCAGCTTGACATCCTTACATGATTTACAAACCTTTTAAAGACCCCATCAGTTTTATTTAT
TTATGATATGGGATAGCTCAGAGATGTGGGATGAAATAAAGCTATTGTGGTGGAGAGGGTTTGAGCCTTGGACTTTGGGGAATG
AAACCCAAGGGATGACCACTTGCCTACTGATCCATTTGTTAAATAGTGCTACGAAAATATCTAAATTAATGTTGGTTTTTTCAAAT
TTCCATTACTTAAAACCCTAAAATAAGCATAAATATTTAACTTTGCATTTATTGTTATAATGATAAATATGAAAATGATTTAAGTTAA
ACCATGTGATAATTTTAAAATTTTGAATGCGAGAAAGAAAAGAGTGTCTTCTTCATCAAGGAAAGAGTACAAAGCATATGTATATG
GACTTTTACGAACTAAACCACCCTTATCTAATAACTATTAATCATTTTTTAAAATAAAATTATCGAAATAATAGGATATACAACTTTT
GACATGTAGATAAAATTAAATAATAGAGTTTTCCTTCCATCTTAAATATGTTTTTACGCTGAAATATTATACATATTTTATTAAATAA
AATTAAAAATATAAATGCATTTATATTTATCATTATCATATTTATCAAGATTAAAAAAAATGAAATATTATTAAGGTTTTAATTATTTA
TATATATTCTTAATTTTTTATAATTATTTTAATTTTAATAACATATAAATGATATATTTATAAAGATATGTTCACATCACGGTTTGATC
ATAGTTGAACCACTAGTTCGACTAGTAAATCATGACCTAATAACTTTTCCGGTTCAATGATCAGATCTGAAAACATTGAAGGAAAC
TTTAGAAAACAACCCCTCTATGTTTGTTTTGGACCATTTGAGAGGAACCTGATCATAGATCCTTTGATGGTGTAGAACAATCAGA
TCAGTCACCTTAAGCACTCTTTTCATTTGTGGTTTCCTCCAAGGGTCTCATCAACATCTTTTTAATGTTTCTCCTTCCTTGTTGGA
CTTCATTGATTGGTTAGGGTCTAGATAGCGCTTTGTGGGGATTGTTTGTTTTTGTTGTTTTGCCCCTTTGGCACCCTTCATATAAG
CTCCCCTGTGTACTTGGGATGTGCCATGCTTTTTATTGATACGATATTCATTTGCCTACCACCCCACCAAAAAAAAAAAAAAAAAA
CCGCACACATGACTTTGCTTGGCATGTTTGATGTGTATCCCTTTTTTCTTTCCTTTTTCAGGTACAAATGTTTGGCCTGCTTCAGG
CAATATAAGAAGAAGGAGCATCTAGTTGAGCACATGAGGGTATCATATCATTCTGTGCATCAGCCAAAATGTGGGGTCTGCCAG
AAGCAGTTCAAATCTTTCCAGTCACTAAGGGAACACCTTACTGGTGAGATACTGCCATGCATTCATTTCTGCACTGTTTATCAGT
AAAACTGCGTTTTTCATTACCTTTCACAATTGTTTAGGTAATTTGCCCAAGAAAAATTGTTTGAAGATATTCTCTGAGCAAGGTTG
CAACTACTGTTTGAAAATCTTCAAGAATGCTGATTCTCTCAGTGAGCATAAAGAAATGTGTCAATTACCTGTGCCTGTTACAATAG
TAAGCTACTCAGTACAATCTTCAAGTACCCCATACATATATAGAGAAATATATCTTCCTGCAAGTTTGTAATGTGGCATGTGATCG
ATTGCAGAAATTCATTTTAAAATCGATATTTTATACAGAATCTCAGATTGATTTCTAAATCTGTATCAAACTCTCTCAGGAAAAAAT
CAGGATGCCATCTACAGAATCTCAAATTGATTTTTCAGATTCTATTAATAGAGACCACACTAGCAGAGGTCTTGAAGCAATTGCC
ATTGACTGTGAAAAGGTTGGTGGCGGAAGTGATGGATCGCTTGACCTTTGTGCTAGGGTATGCCTCATAGATGAAGATGAGAAT
ATAATTTTTCACACTTATGTGCAGCCACAAATTCCTGTCACCAATTACAGGTATTTGTGTGCTCTTTATTATGTTGGGTTTCTCTGA
GATACTGTGGAGAATTGGTTCACTGATCATTCAAATATCCCTGCAAATATTTTCAGATATGAGTTAACTGGGATTACTGAAGCAC
ATCTGAGAGATGCCATGCCACTAAAGGAAGTGCGGGAAAAAGTTATGGAAGTTTTACGAAATGGAGAATCCATTGGGCGACTG
CGTTTGGATGGTGGGAAGGGCAGACTTCTTGTGGGTCATGACCTACGCCATGACCTGGATTGCTTGAGAATGCACTATCCTGAT
CACTTGCTGAGGTGAACTACTTTGGACTTGGCTAATTGAGTAAAGCTGTTCAAATGATATTTTATTTCATTTTGAGAAAAGCTCAT
GATTAAAGCGATGGTTTATCATCTAAAGTCGTACATTTATCTAATGAGTTTATCCATATGATATTTTCCCCCTTCTTACCTCACTTT
TAGATATATCTTGGGAAGTTACTTGTTTCTGTAGTACTGTGTGAAAGTATGACATGTAGTCCATCTAAAACAGTATGGAATAATTT
CCCATTTTAAGGAACAGCTCAACCCTTACTAAATGGTACATGATTGGAGGAATACAAAGGAAGTCATAAGAAAATGATATTATCC
CTGTTCCTCATCCATACCAGGCTAGCAAATGTGAATGCGATTCTGCTGGGTCTTGGTTGAAAACAGATCTCTGGTAAGCGAAAT
TGTATCTTTAGTTTTTCCTTCTAGTCCCCTCCTCATCAACATTATTCCATGATCTCTGGATAGTCAAAAGTATTAGGAAAATCCCTA
GCATCACTATTTCTTGATGAGCTTTTTTCAGGCAGTTGTTTCGAGATAGTTTTTGTTTTTCTTTCCAAGCCCCTGAAAATCAAAGA
ATTGTTGCCTCAATTATGGAAGAAGGTTTGGAAGAATATGTGATTTAATAATAACACCATGGAACTTAGGTTTAAAAGCATTTGCC
AAAGATAAAAGTTGCTGAAGTAATTGGCATGCTCAAAGTAAACTTCATTTGCTTGGCTGCAGCACTTTAGGTTCTGTCTTGGATA
TGCGTCTCCCCTGATGGTTTGATGTGGCAATATTTTCAAAGCATAATCTTTGTTTCAGAAAAATACATCCAACTCTTTTTGTGTTAT
GGTTTGTTTCCTCTTCACATAGGGATACTGCAAAATACCTCCCCTTGATGAAAACAAATTTAGTCAGCTTCTCCCTGAAGTCGCT
CACTCAAAAATATCTTGGGTAAGTTATTGCTCAGGTGCTCTTGAAATTTATTTTCTATTTTACTTTGATTTAGTGTTTCTGAAGTTG
CTTGGTCAGACTATACACTGCATCCATATTGTAGTTTTAACATTTTTCTTTGCTTTTAAAGTAAAACTCAAATCAGGATAGCCACAA
GTTTTGTCCACATTTAGAAACCGGTTGTTAAATGTAAAGAGAATGCCGCAAATTTAATTCATTTTCCCAATATGACTTTTGGAAGG
CAACTTGTAAGTTTTATTCTGTTTCAGTTTGGGATTGTTGTAAGCTTTTTCTGTTTTTCTATCATGTTAATGTAGAAAACGAAGAAG
AGGGGGGACATGGGGTGGTGGTGGATGTTCATTATCATTGTACATATCAAATATTTGGGTATTTATTTATGAAGTTGTAGGGTTC
TCCATCACGCTGCCGGTTGAAAAATAAAAATCTATCTGCGATCCTCATGATAAATACAATTTGTTTGCTTACAGCACTAGCTCAGT
TTTTAAATGTTTACAGGTACGATATCCAGTCAGGGATGCATGATCCATATGAAGATTGTGTATCTGTAATGAGGCTGTACAAGAG
AATGCAATCTCAAGATCACTCAGACGAAGGAATTGGAACAGCCATTGTTAATCACCATACCCAAAGTTTTGGCAGTGGTTTTGAT
TCCTGGAAACTTAGGGAGCTCAAGAATATGACCCCAGATGAGCTTTATAAGCTCTCAAGACCAAACTACAGGTGTTGGTGTTTG
GACTCAAGACAAGCATTACAGGCTGAGAGTAATGATCTTTGGGCACAATAGTGAGTAGATTTCTTTACCTACATTTCAACAAGTT
ATAGTTACCTAACTATGAATGGAAATCATTTAGGTATTTTTTTTCTTAAGATTCATTGATAGACAATTGAACTCTTCCAACATTTTAC
AAAACATACCATTCTTCAAAATTTAGGACCAAAGCTGTTTTACACTGACATGTTTCTATGGCTGAGTTTTTGTTGCACTTTCTTTGC
AGGAACTGATACAGGGGGTTCGATGAGAACATGACAGGAAAAGAAGCGAATATCTCCGGTGAGTCTACAACATTGTAGCCATG
AAATGAATGTGCTACAATGTTGAAGATATTTCGTCGAATATGAGATCATTATGAGAACATTTCCTGATATAGCAAACTCAATATTG
ATAGGGCTTCTTCAATACCCAACCCTGTTTTCCTATGCAAACATTAAACAGATCTTGTGCTTGTGTGTGTGTGTGTGTGGGTTTG
ATTGTTTTGTTGGAGTTGCCTTTTTATTATTTTTTATTTTTTAAATATTTTTATTTTGCAAATACTTTCCCATGTAGTGAAGAATGAG
GAAGAGATCTTATATAATTTCGCATGTAAGGGGTTA
SEQIDNO: 343
MDSRNESSETLRNKCAACFRQFNRMEHLVEHMRISYHSVHEPMCGICKKHCRSFESLREHLIGPLPKQECKNIFSIRGCKFCLNIFDS
YSSRMIHQERCQLSNLSTGLIARMSSLGLRDHLMIDNGYAVGNQVVALACKMVGGGSDGSLDLCGRVCLIDENENIIFHTYVKPPIPV
TNYRYESTGIRPEYLRDAMPLKMVQSKIQNFLCNGEPMWKIRPRGGKARILVGHGLDHDLDRLQLEYPAVMIRDTAKYPPLMKTSKL
SNSLKYLTQAYLGYDIQTGIQDPYEDCVATMRLYMRMRYQRHAVEAYPFASDPQNRNNFASWRQAELERVSPEELLAISRSDYYCW
CLDS
SEQIDNO: 344
CTTGCCTACCTAAATTTTCAAGATTTGCACTACTGCTTCCACAACTCTCATGGACTCTAGAAATGAGTCATCTGAGACTTTAAGGA
ACAAGTGTGCAGCTTGCTTCAGACAATTCAACAGAATGGAGCACTTAGTGGAGCACATGAGAATCTCCTATCATTCTGTTCATGA
ACCCATGTGTGGAATCTGTAAGAAACACTGCAGATCCTTTGAATCCTTAAGGGAACATCTTATAGGGCCACTGCCTAAACAAGA
ATGCAAAAATATCTTCAGTATCCGAGGATGTAAATTTTGCTTGAACATCTTTGATAGCTACAGTTCTCGCATGATTCATCAAGAAA
GATGCCAGCTATCAAATCTTAGTACTGGACTAATTGCTCGCATGTCTAGTTTAGGACTGCGAGACCACCTCATGATCGACAATG
GCTACGCAGTAGGTAATCAAGTAGTTGCTCTGGCATGCAAAATGGTAGGAGGAGGCAGTGATGGCTCTCTAGACCTTTGTGGA
AGGGTTTGCCTCATTGATGAGAATGAAAATATTATCTTCCACACTTATGTCAAACCACCAATTCCAGTGACCAATTACAGGTATG
AAAGCACAGGGATTCGGCCCGAGTACCTGAGGGATGCAATGCCATTGAAGATGGTTCAATCTAAGATTCAGAACTTCCTTTGCA
ATGGTGAACCAATGTGGAAAATTCGACCCAGGGGAGGAAAAGCAAGGATTCTTGTTGGTCATGGTTTGGACCATGATCTTGACC
GTCTTCAACTAGAATATCCAGCTGTAATGATCAGGGATACAGCAAAATATCCTCCATTGATGAAAACAAGCAAGCTCAGCAACTC
ACTCAAATACCTAACTCAGGCCTATCTTGGATATGACATTCAAACTGGCATACAAGATCCATATGAGGATTGTGTTGCAACAATG
AGACTTTACATGAGAATGAGATATCAGCGACATGCAGTAGAAGCTTACCCATTTGCTTCAGACCCACAAAATCGAAACAATTTTG
CTTCATGGAGGCAAGCTGAGCTTGAAAGGGTGAGCCCAGAAGAATTGCTTGCAATATCAAGGTCTGATTACTATTGTTGGTGCT
TGGATTCTTAGAGGAAATGGATGTTAATTCCTACAGGAACCAACTTAATTCATCAACACACATGATTTATGTACGTGTGTTGACCA
TTGGAAACAAGGACTTGAGAACTATCCAATACTTAAGATAGCTAGGAAGAATAATTAATAAGTTGGAGATATATGATGTATGTGTT
TCCATTAGCTACTTAGTACTTACAGTGTAGTAGTGTCATTTAAAACTAAATTTATATAAGCTGCTTAAAAATAAATGGAATCCAACT
TATTTATTGGCTGA
SEQIDNO: 345
CTTGCCTACCTAAATTTTCAAGATTTGCACTACTGCTTCCACAACTCTCATGGACTCTAGAAATGAGTCATCTGAGACTTTAAGGT
GCTCTTTCCCTCTCCTTCTCTCTCTCTCTCATTTCTTGGCACACAGTTTTATCTCACATCAATTAGATATGTAGGTAATGTGAACTT
AAAAGTAAATTTTTCTCTAAATTAGCTAGATTTTGGAGATAAATTAGGCCGGTTCGATTTTTTTATCATACTATAAAAGTTTTCAAA
TTTCATACTCTAACATATTAAAATTTTTCAGTATCGATTGGAGATTCTGATACTAAGTTAAATTTAATTAAGGAAAAAAGTATTTAAA
ACTTTATAAAAAAGACATTATCTTACCATAAATCGATGTGGGATTAAACAAAGATGAGTTATATTATAAGAATAGCTCTTTCTATTT
GAAATATTATACTGCAACCGGTGAAAAATTTCACCACGGATGAGGGTTCTCGACATGCATGCATGATCCCAATGATGCTATATGT
ATAGTGCATGACTATAATAGTACCACATGCAGGAACAAGTGTGCAGCTTGCTTCAGACAATTCAACAGAATGGAGCACTTAGTG
GAGCACATGAGAATCTCCTATCATTCTGTTCATGAACCCATGTGTGGAATCTGTAAGAAACACTGCAGATCCTTTGAATCCTTAA
GGGAACATCTTATAGGTAAACTTCAATCATAAATTCTCCTCACTCGTATAATCTCCAGTTTTCTCTCATTACAAGGGTTTTTAATAT
CAAGGGTCTAATTTGTTAATTTCTACAGGGCCACTGCCTAAACAAGAATGCAAAAATATCTTCAGTATCCGAGGATGTAAATTTT
GCTTGAACATCTTTGATAGCTACAGTTCTCGCATGATTCATCAAGAAAGATGCCAGCTATCAAATCTTAGTACTGTATGAATCATC
TTCACCCTTGTGAATTATTGTTATTACACTTCCATATTAATTAATGATGAAACCCCTTTTGCTAATTTCCCTAATTGGTTTTGTTGTT
TTTTTGTAGGGACTAATTGCTCGCATGTCTAGTTTAGGACTGCGAGACCACCTCATGATCGACAATGGCTACGCAGTAGGTAAT
CAAGTAGTTGCTCTGGCATGCAAAATGGTAGGAGGAGGCAGTGATGGCTCTCTAGACCTTTGTGGAAGGGTTTGCCTCATTGAT
GAGAATGAAAATATTATCTTCCACACTTATGTCAAACCACCAATTCCAGTGACCAATTACAGGTATTGATTTTAACTTAGTCATGT
TAGAGTCATCTTTGCACTTATAAACTTTCGAATTTAAGTCTCAATCGGAGCTAAATAAAAAAAAAAAAAAAAAAAAGAAAATTATCA
AGAGTCTTTGATCGATTCTTGCAAAACTTTGCAGGTATGAAAGCACAGGGATTCGGCCCGAGTACCTGAGGGATGCAATGCCAT
TGAAGATGGTTCAATCTAAGATTCAGAACTTCCTTTGCAATGGTGAACCAATGTGGAAAATTCGACCCAGGGGAGGAAAAGCAA
GGATTCTTGTTGGTCATGGTTTGGACCATGATCTTGACCGTCTTCAACTAGAATATCCAGCTGTAATGATCAGGTAATAAAGGTC
CTCCCGACAGCTAAAAGTTTCACCCTGTTTCCTTATAGCTCTCTCCCTAGCAATTATAAAGTAAAGCGGTTATATCAAGTAGGATT
AACTATACAGTACAAATTAAAGATAGACAACTATATAAATATAACCTAGTATAAATGAGTAAACCCTAATAATTTTTTTTCAAAAAA
AAAAACTATTTTTATATAATTATTTATTTGTTATTGACTTATTATACTATATAGACAATTTTATATGATATAATGGCTTTTACGGTATA
ATTTTTTCTCACTCTTCTTTTTTTTGAATTGTTTTGAGAAACCCGTTAATATAAAAGCACCTTGGATGGATTCCATTACAGGGATAC
AGCAAAATATCCTCCATTGATGAAAACAAGCAAGCTCAGCAACTCACTCAAATACCTAACTCAGGCCTATCTTGGGTATGTATTA
TCTTTTTTTTTTTTTCCCTTCTACATGTTTAGTGTGCCTAAAAGTTTTTAACGCACAGATATGACATTCAAACTGGCATACAAGATC
CATATGAGGATTGTGTTGCAACAATGAGACTTTACATGAGAATGAGATATCAGCGACATGCAGTAGAAGCTTACCCATTTGCTTC
AGACCCACAAAATCGAAACAATTTTGCTTCATGGAGGCAAGCTGAGCTTGAAAGGGTGAGCCCAGAAGAATTGCTTGCAATATC
AAGGTCTGATTACTATTGTTGGTGCTTGGATTCTTAGAGGAAATGGATGTTAATTCCTACAGGAACCAACTTAATTCATCAACACA
CATGATTTATGTACGTGTGTTGACCATTGGAAACAAGGACTTGAGAACTATCCAATACTTAAGATAGCTAGGAAGAATAATTAATA
AGTTGGAGATATATGATGTATGTGTTTCCATTAGCTACTTAGTACTTACAGTGTAGTAGTGTCATTTAAAACTAAATTTATATAAGC
TGCTTAAAAATAAATGGAATCCAACTTATTTATTGGCTGA
SEQIDNO: 346
MASRNESSDTLRNKCAACFRQFNKMEHLVEHMRISYHSVHEPICGICKKHCRSFESLREHLIGPLPKQECKIIFDVVGCKFCLTILDSY
NSRMIHQGRCQLSNGLIARMPNLGLRDNLTIDSGYTRGPQVVALACKMVGGGNDGSVDLCGRICLIDENENIIFHTYVKPPVPVANFR
YESSGIRPEYLRDAMPLRLVQSKVQNFLCNGEPLWKVRPRGGKARILVGHCLDHDLDLLQLEYPAMMIRDTAKYPPLMKTSKLSNSL
KYLTQAYLGYDIQTGIQDPYEDCVAIMRLYMRMRSQKHAPEDYPLASDPQNRNNFASWRQAELERMSPEEMLAISRSDYYCWCLDS
SEQIDNO: 347
CTCAGGATCAACACTGCTACTTCCACAACTCTCATGGCCTCTAGAAATGAGTCATCTGATACTCTAAGGAACAAGTGTGCAGCTT
GCTTCAGACAATTCAACAAAATGGAACACCTAGTGGAGCACATGAGAATCTCCTATCACTCAGTTCATGAACCCATTTGTGGAAT
CTGTAAGAAACACTGCAGATCCTTTGAATCCTTGAGGGAACATCTTATAGGTCCACTGCCTAAGCAAGAATGCAAAATTATTTTC
GATGTCGTAGGATGCAAATTTTGCTTGACCATCCTTGATAGCTACAATTCTCGCATGATTCATCAAGGAAGATGCCAACTTTCAA
ATGGACTGATTGCTCGCATGCCTAACTTAGGCCTGCGAGACAACTTGACAATTGACAGTGGCTACACAAGAGGTCCTCAAGTAG
TTGCTCTTGCCTGCAAAATGGTAGGAGGCGGCAATGATGGCTCTGTAGACCTTTGTGGAAGGATTTGCCTTATTGATGAGAACG
AAAATATAATCTTCCACACTTATGTAAAACCACCAGTTCCTGTTGCCAATTTCAGGTATGAAAGTAGTGGCATTCGGCCTGAGTA
CTTAAGGGATGCAATGCCACTGAGGTTGGTTCAATCCAAGGTTCAGAACTTCCTTTGCAATGGAGAACCCTTGTGGAAAGTTCG
ACCCAGAGGAGGAAAAGCAAGGATTCTTGTGGGTCATTGTTTGGACCATGACCTTGACCTTTTACAACTAGAATATCCAGCTAT
GATGATCAGGGATACAGCAAAATATCCTCCATTGATGAAAACAAGCAAGCTCAGCAACTCACTCAAGTACCTAACACAGGCCTA
TCTTGGGTATGATATTCAAACTGGCATTCAAGACCCTTATGAGGATTGTGTTGCAATAATGAGGCTTTACATGAGAATGAGATCC
CAAAAGCATGCACCAGAAGATTACCCACTTGCTTCAGATCCACAAAATCGAAACAATTTTGCTTCATGGAGGCAAGCTGAGCTT
GAGAGGATGAGCCCAGAAGAAATGCTTGCAATATCAAGGTCTGATTACTATTGTTGGTGCTTGGACTCCTAGAGGAAATGGATG
TTAATCCTAAACAATGGAGCTAAAATCCTTCAACACATATATGATATATATCAGTCCAATGGTGGAAAGAATTACCATAATTATCC
AATATTTATTACAGCTAGGAAAATAATTAATAAATTGGAAATATATTATATGCATATTTCCATGAGCTACTTAATTAATTATTCTGTA
GTAATCCATGCAGCTGA
SEQIDNO: 348
CTCAGGATCAACACTGCTACTTCCACAACTCTCATGGCCTCTAGAAATGAGTCATCTGATACTCTAAGGTTCTCTCTCTCTCTCT
CTTTCTCTCTCTTGTGTAGCACACATATGGATGTGTTTGTCTAGATCCCTACCTTAAATTATTTACTTCAAGATAGATTAGCATCCA
ATGTTTAGGATCAGGATGGTTTCTTTCATTTGTGTGGATCTTAGAATGTATTGTCATACTATGTTTTTCTTGGATTCTAATAAAACT
TTTCTTATAAAAAAATTATAACGTATTGTTTCGTATCCATTTTTCATGATCGGTATGAACATAAATATATTGAATGCCAAACCATAA
AATTTATATGCTTTTCCTTGTACTATGTTTGTGCACATTACTAGAAAATTTTATATTAACTAATTGTTGTATATACTATGATTAACGG
CTATAATATATAATCTTCGAATATACAAAAATTGTATTATAAAATTTCGTTAGACTCCGATTAAATAGGCTTTCCGAACTAAATGAA
TATAGGATTTTTTGATAGCATGATATTAGTTATTGAGGTATAATATATATAAAGTGCCAAAACTAATAATATTTGCATTTGTATTTG
CAGGAACAAGTGTGCAGCTTGCTTCAGACAATTCAACAAAATGGAACACCTAGTGGAGCACATGAGAATCTCCTATCACTCAGT
TCATGAACCCATTTGTGGAATCTGTAAGAAACACTGCAGATCCTTTGAATCCTTGAGGGAACATCTTATAGGTAATAATTTTTCAG
TAAGAACTTATCCTCACTCAGATAATCTCCAGCTAGTTTCCTATTATTATTAAGGGTTTAATTTGCATGCTTTGCTTCTACAGGTCC
ACTGCCTAAGCAAGAATGCAAAATTATTTTCGATGTCGTAGGATGCAAATTTTGCTTGACCATCCTTGATAGCTACAATTCTCGC
ATGATTCATCAAGGAAGATGCCAACTTTCAAATGTTAATGCTGTATTGATCGTCAACTTCAGTACCCTTTATTAATTAATTAGCAAT
TTCCATAGTCTAGCTAAGTATTTGCTGGGTTGTTGCTTGCTTTAGGGACTGATTGCTCGCATGCCTAACTTAGGCCTGCGAGACA
ACTTGACAATTGACAGTGGCTACACAAGAGGTCCTCAAGTAGTTGCTCTTGCCTGCAAAATGGTAGGAGGCGGCAATGATGGC
TCTGTAGACCTTTGTGGAAGGATTTGCCTTATTGATGAGAACGAAAATATAATCTTCCACACTTATGTAAAACCACCAGTTCCTGT
TGCCAATTTCAGGTAACAATATTGCGTACTATGTTTTTTCGAAAATCAAAATTTGAACTGCTATGTTACTATTATGGAATTTTCTGA
TTCGGAGAGATTGCGAATGATTGTTGTGAAGGTTCATATATTGAGCTTAGGGTTGCTTCTATCAAAATTTTTTTAATGTGGGACAT
TCAGCAACCAGGCAATGTAAATGTTAGCTGTGTTTATTGATTCTTGCAAATACTTTGCAGGTATGAAAGTAGTGGCATTCGGCCT
GAGTACTTAAGGGATGCAATGCCACTGAGGTTGGTTCAATCCAAGGTTCAGAACTTCCTTTGCAATGGAGAACCCTTGTGGAAA
GTTCGACCCAGAGGAGGAAAAGCAAGGATTCTTGTGGGTCATTGTTTGGACCATGACCTTGACCTTTTACAACTAGAATATCCA
GCTATGATGATCAGGTAAATAAAGCTTCTACTGCTAGAAGCTAGCCAAGTTGCAACTTTTTTAAATGAAAACTCATTTCTTTTCAA
TTATTCTGAGTAACCTTGTGAGAAAAAGTATCTTGAATGGATTCCATTACAGGGATACAGCAAAATATCCTCCATTGATGAAAACA
AGCAAGCTCAGCAACTCACTCAAGTACCTAACACAGGCCTATCTTGGGTATGTACTCTTGTTTCCTCTTCGTCTAAATTTCTCTTA
TATAACATATACTTTAATATCATCTGCCTATATATTTAAGGAAAAATTATCATTTGGTCCATGAAATATAGAAAAACCCATTAATTA
GTCCCTTAATTTAAAAAAAATACATTATAATGTCATTATAATTTTATAGGTATGATATTCAAACTGGCATTCAAGACCCTTATGAGG
ATTGTGTTGCAATAATGAGGCTTTACATGAGAATGAGATCCCAAAAGCATGCACCAGAAGATTACCCACTTGCTTCAGATCCACA
AAATCGAAACAATTTTGCTTCATGGAGGCAAGCTGAGCTTGAGAGGATGAGCCCAGAAGAAATGCTTGCAATATCAAGGTCTGA
TTACTATTGTTGGTGCTTGGACTCCTAGAGGAAATGGATGTTAATCCTAAACAATGGAGCTAAAATCCTTCAACACATATATGATA
TATATCAGTCCAATGGTGGAAAGAATTACCATAATTATCCAATATTTATTACAGCTAGGAAAATAATTAATAAATTGGAAATATATT
ATATGCATATTTCCATGAGCTACTTAATTAATTATTCTGTAGTAATCCATGCAGCTGA
SEQIDNO: 349
MDSDSYPPTKALTTRYKCYACYKQYKKKEHLVEHMKISYHSAHQPRCAVCQKHCKSFESLREHLTGPLAKTNCLGIFSDRGCDLCLE
VFDSPSSLNKHREMCCLSAPASLATEIPTCTESQIYVSGSIDESYACKGGEAVAIDCEMVGGGSDGSLDLCARVCLLDEDENIIFHSYV
QPQIPVTNYRYEVTGLTEKHLRDAMPLKEVQNKILEILYNGESIGKLRLSGGNARLLVGHSLDHDLDCLRMFYPDHLLRDTAKYRPLM
KTNLVSHSLKYLVQTYLGYNIQTGVHDPYEDCVSVMRLYKRMRAQNHHVEGSGIQSICGGFDFWKPKELEKMTPEKLYEISRSNYRC
WCLDLKGQQPGLI
SEQIDNO: 350
CCAGTCTCCTTTCCAAGTCCCGGCTGTGTTCAGTCCCTGTGAATCCTTCGGCTTAACTTTTCCTATTGATCAAGAAAAAAAATAC
CCCACTGATTTTAGCGGATTTCAGCTACATGGATTCCGATTCTTACCCACCAACAAAAGCCCTAACCACAAGGTACAAATGTTAT
GCATGCTACAAGCAATATAAGAAAAAGGAACATCTTGTTGAACACATGAAGATCTCGTACCACTCAGCTCATCAGCCCAGATGT
GCAGTTTGTCAGAAACACTGCAAATCTTTTGAATCTCTGAGAGAACATCTTACAGGTCCGCTGGCAAAAACAAATTGTTTGGGTA
TTTTCTCGGACCGAGGTTGTGATCTTTGCTTGGAAGTTTTTGATAGCCCTTCTTCTCTTAACAAGCACAGGGAAATGTGTTGCCT
TTCTGCACCTGCTTCCCTTGCAACTGAGATACCAACTTGCACAGAATCACAGATTTATGTTTCAGGATCAATTGATGAATCTTATG
CTTGCAAAGGTGGTGAAGCTGTTGCTATTGACTGTGAAATGGTTGGTGGTGGAAGTGATGGATCACTTGATCTTTGTGCTAGGG
TGTGCCTTCTTGATGAAGATGAAAACATAATTTTCCACTCGTACGTTCAACCTCAAATTCCTGTAACCAACTACAGATATGAAGTA
ACTGGGTTAACTGAAAAACATCTAAGAGATGCCATGCCGCTTAAGGAAGTGCAAAATAAAATATTAGAAATTTTGTACAATGGGG
AATCTATTGGAAAACTAAGGTTGAGTGGTGGAAATGCAAGGCTTCTTGTCGGTCATTCACTGGACCATGATTTGGATTGTTTGAG
AATGTTCTATCCTGATCATCTGTTGAGGGATACAGCAAAATACCGTCCTTTGATGAAGACTAATCTGGTTAGCCACTCACTGAAG
TACCTTGTGCAAACCTATCTAGGATATAATATTCAGACAGGGGTACATGATCCTTATGAAGACTGTGTGTCAGTGATGAGACTTT
ACAAGAGAATGCGTGCCCAAAATCATCATGTTGAAGGTTCAGGGATACAAAGTATATGTGGTGGTTTTGACTTCTGGAAACCCA
AGGAACTTGAGAAGATGACTCCAGAGAAGCTATATGAGATCTCAAGATCAAACTACAGATGTTGGTGCCTGGACTTAAAAGGCC
AGCAACCCGGCTTAATATAGATCTTCTGAACAAGTAGAAAAATTGAGAGGTTCAACAGCTGTAAATGGGATCCTGCAGTTCTTTC
TCGCCTTTTGGTTTTTGAGGAGTCTGCCAAGCTATGGTGGCTATTTTTGGCTGCTCACAGGTTATAAACATGACGTCTAAGATTT
GAAATAGTTTCTTAAAATTAATTATTGATCAGTATGGGTGTCTGGGTGGAGAAGAAAATCTTGGTCCCAAGAGTTTCTGTACAGT
TTCATAGATATAATAGTATATATCTTTGATATTATAACTTATTTTAATGAAATTTTATGGTCATGTAATGTTTTCTTTCACCGCTGAT
TTT
SEQIDNO: 351
CCAGTCTCCTTTCCAAGTCCCGGCTGTGTTCAGTCCCTGTGAATCCTTCGGCTTAACTTTTCCTATTGATCAAGAAAAAAAATAC
CCCACTGATTTTAGCGGATTTCAGCTACATGGATTCCGATTCTTACCCACCAACAAAAGCCCTAACCACAAGGTTCTTCCCTCTG
TCTCTCTGTGTGTTGCTGCTTGGTTTGAAGGGAAAATGTGGAAAAGGAATGAATTTTACTTCTCGTTTTCGCTGTATTTGCGCGT
GTACTGTATACAATTCCCTGGAATAATGATCATTTTAGGCCTCCTCTATATTCTACTCAAGTGTAGTTGTTTCTACTAATCCAAGTA
GACTAACCTTGGCGAGTACCATAATAGTAACTTACATTTAGAGTGTAAAATCAATTGCAGACATGCCTGTATTAGAGTAATATCAC
GATGTGATAGAAAATTGCTGAACATATCATTATGCATTGTCAATAATACATATCCATCCATCAGGAAATTTCCAGAATTTTCCATAT
GGAATTTCAGACGAATTTCTTTGCAGAAAGACATATACCTTCCGTTTAGCATGATTCATTTTACAATAAAAGATATCAAATGAATT
CTTGCAAGATTTCTACTTCTCTTAAAATGATTTTTTTTTAAAGATAAAAAGAATTGTATTCTTTCATTCCATATAAGATAATTGTTAG
AAAAGAAATCAATTGAATTGAATTCTTATAAAATTTTTTATTCCAAACAGAGGATAATTTTTTAAAAAAAACAGAGGGATAATGCTT
AGTTGCATATTTTGTTATTACTTGTTATATTAATCAATAAACTATATGACTGACCAACTTCAGTTTCCAGTATATAAAATTGGCATTA
TTGTTTTGGATTCTAGAGTGATGAGTAGATATCTTGGTCTGATTTAAATAGGATAATTGAAAATAATGTATTTGTTTGCAGGTACA
AATGTTATGCATGCTACAAGCAATATAAGAAAAAGGAACATCTTGTTGAACACATGAAGATCTCGTACCACTCAGCTCATCAGCC
CAGATGTGCAGTTTGTCAGAAACACTGCAAATCTTTTGAATCTCTGAGAGAACATCTTACAGGTGTGGTTCTATTCCATGTTTATC
CTGCTGTATCTGTTAATAAGTTGATTATTTTGGCTTATACTCTTATCGTTTAGGTCCGCTGGCAAAAACAAATTGTTTGGGTATTTT
CTCGGACCGAGGTTGTGATCTTTGCTTGGAAGTTTTTGATAGCCCTTCTTCTCTTAACAAGCACAGGGAAATGTGTTGCCTTTCT
GCACCTGCTTCCCTTGTAAGTGGATTGTTAAGATTTGAAGGATTCCTTGAAGAAATTAAGAAACCAATCTTTTTATATTGCTTCTA
GTTTATTTTTCTCTTTTAAGGGGTCAATTAGTGAAGGTTATCATCATCACTTGCTTTTAGGTGCATAAATTTGGAACTTTCTGTTGT
TTATTGTAGGCAACTGAGATACCAACTTGCACAGAATCACAGATTTATGTTTCAGGATCAATTGATGAATCTTATGCTTGCAAAG
GTGGTGAAGCTGTTGCTATTGACTGTGAAATGGTTGGTGGTGGAAGTGATGGATCACTTGATCTTTGTGCTAGGGTGTGCCTTC
TTGATGAAGATGAAAACATAATTTTCCACTCGTACGTTCAACCTCAAATTCCTGTAACCAACTACAGGTACTTTTAAAATCCTATG
TTATCATTTAATTCTTCAAAATACAGAAAGCATTATTAGGTATATTGTCATTCACATCTATTCAAATGTTTCAGATATGAAGTAACT
GGGTTAACTGAAAAACATCTAAGAGATGCCATGCCGCTTAAGGAAGTGCAAAATAAAATATTAGAAATTTTGTACAATGGGGAAT
CTATTGGAAAACTAAGGTTGAGTGGTGGAAATGCAAGGCTTCTTGTCGGTCATTCACTGGACCATGATTTGGATTGTTTGAGAAT
GTTCTATCCTGATCATCTGTTGAGGTACATTTATATTAGATAGACTGACATGATATAAGATTAGCTCATTTAATGTTCCATGATGAT
GAAGTTATTTTGGATGAATCCTTATGGTACTTGATACATATTATACATATCTCCATTTGGCAAAAATCAATTTGCCCATTTCTGGG
GGGAAGGAATCCTAAGATATTACCATCAGTGACCTTGTCTGGGACTTGGTTAATATCATAGACATTTTACTGGTATGTAACCAAG
TCATGTTTCCTCACATTAATTGGATACAGTATCTTTTCTTTGTGTATCAAGCATCACTGGGATTCTGGAATTGATGATTTTGCAAT
GACTGTTTTCTAGTGTAATAACTGTCCAATATCCTTTCAGAAATATGTGGACATTCCTAATTGCTTATGTGATTATATTATCATAAG
GGCAGTTCTTTGAGGCATTAAACACCTTCCCATCAGCGGCTGGCAATCTTTAGAATCATCAGAATAGTTCACTATTTTTTGGCAA
GTCTTATATGAGCAATCATAATGAGTTTGTCTTGGTACTTGCAAACTAAAAATTGATTGTTGTTAACGTAAATACTGAGGAGCTCT
ATTGAGTAAATAAATAGAGGAAATAAAGTAAACAGGTTTAGTAAAGTTCTATTGATAGATTTTCTTCCTTCAGATATTTATATCTGG
AACAAATAATTTGTACTATTCAATATAATTCTTTTTTTGGTGTGCTCTCCCCAGTTTTACATACTTTAGCAGTGTTACACTTTCCGC
CAGTTGACCAAAAGAGTCTTGATCTTTGTGTGGTTTTTCAGTTTTGGCCCAACCAGTTAAACTTTAATTGGTTTTATTGTGTCTAA
TATAGTGAGGTTATTTGAAATGTGTATTTAGGAAGCTCGGCTGCTAAAATTTACTTATTTTCTGTATGGTATTAAATTTTGATCATT
TAGGGATACAGCAAAATACCGTCCTTTGATGAAGACTAATCTGGTTAGCCACTCACTGAAGTACCTTGTGCAAACCTATCTAGG
GTAAGTCACTCATCCTTTCTGGTTTATTGCTCATGCTGATGTTGAATTACTCTACAATGTTTATGCATTTATTTTTTGCTCTTCTTC
TTTTTCCCGATTCCTTTTATTATTACCTATTTGAATTCCTCTGCGCTATCTATATTGCTGTTTCTTTTTTTGGAAGTCAAATGCTTGA
AATGACATACTTTAACTCTTTGTTATACACTCTGATTTTACAATGCAGATTTTATGCCTTTTAGGATTATCCATTCCAGTGGAATGT
TTTGCATTATGATGATGCTTAAAAGAATGAAGTAACATGTTCATATACATTACAATACTAAATGTTTGTGGTCCCAGAATTTGTTTA
AGAGACCAACAAGTTTTCTAAACTTGTAGTGGAAAAGACTGGAACAATATCCAAACAGGAATTTTGGGGAAGTCGGTTGAAGTA
GAGTTTAAGATTTGGCAAAAAAGACTTTTATGTCTTCTTTTTTTTTTTTATTGTGGAAGTGGTGGGTGTGTTGGGTAAGTCTCTTC
CTCTTTGATGCTGTTCTTCTTTGACATGAGAGGATAGATTACCTAAAATCACATATTTCTTCTGCTTGCAGCTAACAGATGTAACT
TTAGCTGAATTTGACAAACATCATATTCTTCCATTTACTTAAGGCCACTCTGACAAGTGACAAAACATCATACTTACTACAAGACA
ACATGCTTACTTTTACCTTTTTTCACTGTTACACGGGCCTTGACTATGTAATCTACCAGAAAAAAAAGCCATCCGCTTGTCATAAC
AAAATTTCTGCTTCACAGATATAATATTCAGACAGGGGTACATGATCCTTATGAAGACTGTGTGTCAGTGATGAGACTTTACAAG
AGAATGCGTGCCCAAAATCATCATGTTGAAGGTTCAGGGATACAAAGTATATGTGGTGGTTTTGACTTCTGGAAACCCAAGGAA
CTTGAGAAGATGACTCCAGAGAAGCTATATGAGATCTCAAGATCAAACTACAGATGTTGGTGCCTGGACTTAAAAGGCCAGCAA
CCCGGCTTAATATAGATCTTCTGAACAAGTAGAAAAATTGAGAGGTAATGCCATTATTTTCCTGCTTTGCTACATGCATTATGGAT
AAGGTCACATACCCCAATCATTATTAAAATTTCAGAACACTTGTTACACCCTTGCTGAACATTACATCCTCGTGTTCAGACCATAA
ACTTTCTTCATTGACATTTGAAGAGGTTTGTATAAATCAATCATTGACTGGCCAGTCTGCCCCTCACAGAGCCTCAGTTCTTTCCA
TGTTAATGATGTATATTGTTGTCTTTATATTTTGGATTTCTTTATTAGATCTCATTTTTACATTCTCAGCGCAATAGTGATTGTATAT
TATATACATTTTGTAGGTTCAACAGCTGTAAATGGGATCCTGCAGTTCTTTCTCGCCTTTTGGTTTTTGAGGAGTCTGCCAAGCTA
TGGTGGCTATTTTTGGCTGCTCACAGGTTATAAACATGACGTCTAAGATTTGAAATAGTTTCTTAAAATTAATTATTGATCAGTAT
GGGTGTCTGGGTGGAGAAGAAAATCTTGGTCCCAAGAGTTTCTGTACAGTTTCATAGATATAATAGTATATATCTTTGATATTATA
ACTTATTTTAATGAAATTTTATGGTCATGTAATGTTTTCTTTCACCGCTGATTTT
SEQIDNO: 352
MESSESMRNKCAACFRQFIRMEHLVDHMRTTYHSVHEPTCGVCKKHCRSFESLREHLIGPLPKQECKNLFNIRGCKFCLAILDSPYAL
RIHQDRCQLSGVNYGIAARLANLGLRDSLTIDNGFSRGPQVVALACKMVGAGSDGSLDLCARVCIIDENENMIYHTYVKPPIPVTNYRY
ETTGIRPEYLRDATPLRQVQRRIQDFLFNGEPMWKIRSPKGGKARILVGHGLDHDLDRLQVEYPAIMIRDTAKYPPLMKTSKLSNSLKY
LTQAYLGYDIQTGIQDPYEDCVATMRLYMRMRNQAHRRENYPLASDPQNRNNFASWRQSELERMSPEEMLATSRSDYYCWCLDSE
SEQIDNO: 353
AAAATCATGACACCAGATTAAAGAAGAATACAAAACCCGACACTCAACATTCCTAGTTACGCAAAGACCAGCTTAGCACTGTCCT
CAAATTAACATATGGACAAGTATCTCAAAGCTTGATTCACTAGAGTGTGATCCCACACTTAGCAAGACGCAACGACAGTGGAGT
AACAGCAGCATCCAGTCCCACAGATATCAGCTGACGACAAGAGCTTCCAAACTCAAACAGAAACCCAGGCCATCCAACCCTAG
CATCAAGAAAGTACCTTGGTATCATTTCAAAGCTCAGCACCAACAGGTATCACAACTCAGCATTCGAATCTAACTTGTGTGGTAA
TTTTTTCAAAGAAGGAATTTTATTGTAATGACTTTTTTCTAGAGTAAGTTTGTTGTGAAAGTGAGGGGATGAACTGGAGATGAGAT
GGAATGGAATGGAATGGAGCTTTAGTGAAGTAAGAGGGAGAGAGGATCTGATCCCCCGGTTAGGTCCTTCTAAAGTAACACAC
GCTAAATTTTCGACGCTCATCATAACAAGATGGAGTCTTCAGAAAGCATGCGGAACAAATGCGCGGCATGCTTCAGACAGTTCA
TCAGAATGGAGCACCTGGTCGACCACATGAGAACTACATATCATTCAGTTCATGAACCTACTTGCGGAGTGTGTAAGAAACACT
GCAGGTCGTTTGAATCGCTAAGGGAACATCTTATAGGTCCATTGCCGAAACAAGAGTGCAAGAATTTGTTCAACATCCGAGGAT
GCAAATTCTGCTTAGCCATTCTGGATAGCCCATATGCTCTTAGGATCCATCAAGACAGATGCCAGCTCTCGGGAGTAAATTATG
GAATAGCGGCTCGTCTGGCTAACTTAGGTCTTCGTGATAGCTTGACAATCGACAATGGTTTTTCAAGAGGCCCGCAAGTAGTTG
CACTAGCTTGCAAAATGGTTGGTGCTGGCAGTGACGGCTCATTAGATCTTTGTGCAAGGGTTTGCATCATTGATGAAAATGAGA
ATATGATCTACCATACTTATGTCAAACCACCAATTCCAGTTACAAACTACAGGTATGAAACCACAGGCATTCGACCAGAATATTT
GAGAGATGCAACGCCATTGAGACAAGTGCAAAGAAGGATTCAAGACTTCCTTTTCAATGGAGAACCAATGTGGAAAATTCGATC
ACCTAAAGGTGGAAAAGCCAGGATTCTCGTGGGTCATGGTCTTGATCATGACCTTGACCGTCTCCAAGTAGAATACCCAGCAAT
CATGATAAGGGATACTGCAAAATATCCTCCGTTAATGAAAACAAGCAAACTTAGCAACTCACTCAAGTACTTAACCCAAGCATAC
CTGGGGTATGACATCCAAACCGGCATTCAAGACCCTTATGAGGACTGCGTTGCCACAATGAGGCTTTACATGAGGATGAGGAA
TCAAGCTCATAGGAGAGAAAACTATCCACTGGCTTCTGACCCTCAAAACCGAAACAACTTTGCATCATGGAGGCAAAGTGAGCT
CGAGAGGATGTCCCCAGAAGAAATGCTGGCTACCTCAAGGTCTGACTACTACTGTTGGTGTTTGGACTCTGAATGAGCATTGAA
CGCCAGCATATATATGTGTAGTTCTCATGTGGTCAAACAGGTGTTCCATTCTGATTAAAGTTTAAGGATGCTA
SEQIDNO: 354
AAAATCATGACACCAGATTAAAGAAGAATACAAAACCCGACACTCAACATTCCTAGTTACGCAAAGACCAGCTTAGCACTGTCCT
CAAATTAACATATGGACAAGTATCTCAAAGCTTGATTCACTAGAGTGTGATCCCACACTTAGCAAGACGCAACGACAGTGGAGT
AACAGCAGCATCCAGTCCCACAGATATCAGCTGACGACAAGAGCTTCCAAACTCAAACAGAAACCCAGGCCATCCAACCCTAG
CATCAAGAAAGTACCTTGGTATCATTTCAAAGCTCAGCACCAACAGGTATCACAACTCAGCATTCGAATCTAACTTGTGTGGTAA
TTTTTTCAAAGAAGGAATTTTATTGTAATGACTTTTTTCTAGAGTAAGTTTGTTGTGAAAGTGAGGGGATGAACTGGAGATGAGAT
GGAATGGAATGGAATGGAGCTTTAGTGAAGTAAGAGGGAGAGAGGATCTGATCCCCCGGTTAGGTCAAAGGGGCAACTTTATT
TATTTTTTTGGACCTTTTTGAAAGGTTCTGATCTCTTTTCTTTCTTTTCAATTTTGACTTTTATTGATTCACTGAAAATTAAGGGGAT
ATAACATATGTGTGGTTCAAAATGCCAAATAGTTTGGCCACAGACTGGCCCCCTCAATGACATTAATTCCTTCACCTCCTTTGAA
ACTAGTCAAAAATAAGCTTTTTCTGTAGTGAACCAAGCTGTGCAGGTTCTATATGAGGGTTACAGTCAGCAGGGTGCATATGGA
CCATTTCTTCATCCACTGTACATGTGCAGTTTTAAGTTGAAGGGTACCCCCATTTATATATCATCAGATTCCCATACCCAACAATG
TTTTCACCAATGATTTTAGAATAATTGCCAATGTAGGTCAGGCATTAAGTTAAAGAACTTTTAACATGATCGATGTCTAGGCGATC
GACATTTTTAAATCAAATATTAATAAATAAAGAATCGAGATGTCTCATCTTTTTATTTTTTTTTAAAAATATTATATCAAATAAATCGA
TATAAATAAAGAAAGACAAAACATATCGCAGTTAATTTTTCTTACTAAAAAGCAAGTIGTGATTAGAGCCCAAGTTTGCCAACAGG
TTACAGCTGCCCCTTTTTATAGCATGCCTAAGGGCATTCCAACATGCATGTAAGTGATTAAAAACCATCATATACACACAGAAATT
TAATGCACAAGCTGCTCCAATAATCTATGTAAGTAAGAGTTATCAGAGGATAAAGAAATTGAATTTATGGCAATTOCCATATATAA
TTTTCAACAGGAGTAGGAACATACACATCTTAGTTGATTTGGAAAAATTTTATCAAATGCTAAACTATGTAAAAGTGTCAATGGGG
AAGCTGTGATTGAGAAAAAGCCAGTTATCTTCGTCGAAGGGAGGGCAATCTCTGGGGTGGATTCTGTTTGACTTGTGAATATAG
AGACGTTTATGGGAATGATGGAAATGGGCTTGTATGGAACAGAATCATTAAGGGAAAAAAAGTGCTCTTTCACCTACAACTTTTC
TGTTGGGTATCTTCTTCAAAAGCAACATGGAGGGAATCTCCACCAGGGTCCCTTTAGAGTCAAAAGTGACTCTTTGCTGAAATTC
CCCATGTTTTGTTTTTCTTAAAGTCAAACCTCTTTCTTTCTTGGAAGGAGAAGTAAGTAATTGTGCTTAAGCTTATGCAAACAACC
TTATCTGCCTTCGCCGAAAGTAAAGGGGCTTAAACTTGAATAACTATGAGGGTTTTACCATGAGTCTCTAAAGCCCATTACCCAT
TGGCATTTGCTTTTGCTGGACAGTTTTTGTCATGTCAGCCAACTTGGTCTTGCCCTTTTTTCCTTCACAGTGCAAGACAAAAGAG
ATAAGCTATGGCATTTTCCAGCTTGAAAAAGACCTTATCCCTGCCTTTGCCGAAAGTAAAAGGAGATTAAACTTGATCACGAACT
TACCCCCCATTTTATGCTGGGCAATTTTATCCTTTTGTCTTGTCAGCCAACTTGGGTTTGGCCTTTTCTCCATCCGCATTCAAATT
CGGGAGTTTGTGAACTTCCTACGTTTGTGATAGAAAAGCAAACGATGGAAACAACGCAATCTTTGACCAAATCTTCATGGAAAAA
GTAAGCCATGGACATTGTTTGCATTAGTGAATAGTGCCTTAAGTCACACGACTTCTTAAAGAAGTATGACCAAAGTAGCTCGTGT
TCATCACTTGCACCAGTCTTGTGTTAAATGCACCACAAAAAACCTCTATAAAGAGGGAAATTCAGTATCAAACTGATGACTAAAA
GTGTAGGCCTCTGATCAATTTATACTACAAAAAGAATAGTTAACATGTTGTTTAGCGCATTAAGTAAAGCTAACCATGCACACGG
GCTTGCGCACGTTCATGTGTAAACTCTCACATACACATGCACACATATAAAAAGCAAGGGAGGGATGGAGGAGACAAAGAGTG
AAATATTCTAGTTGATCAGTGGGGCTCCAAGCATCTGGATTACTACGAATGTTCTGGTGCAGAGATAGAGCTTCAAAATTCAAGT
AATTTTCCTCAAGGAATCATCCTTAGGCTCAAGTGGGACACAAATTTCTTCCTTAACTGATATAAATACCCTCCCCTATTCCCTCT
TCTTTCCTCAGGTCCTTCTAAAGTAACACACGCTAAATTTTCGACGCTCATCATAACAAGATGGAGTCTTCAGAAAGCATGCGGT
ATTCTCTCTTCTTACCTGCTCACACAAACATGCAAACACACACAGGCATGCACACATAAAATCACACTAACATATGTATATAAGCA
ACAAACAGAAAGCACTTTGTAGATAAATACTATAGCACAAATGATTTTAATGCTAAATTGATCAAATTATTCGAGCTGCAGGAACA
AATGCGCGGCATGCTTCAGACAGTTCATCAGAATGGAGCACCTGGTCGACCACATGAGAACTACATATCATTCAGTTCATGAAC
CTACTTGCGGAGTGTGTAAGAAACACTGCAGGTCGTTTGAATCGCTAAGGGAACATCTTATAGGTAAGTAATAAATACTAGCACA
TCCATAGGTCTTGTTAATCCAAAGGTTTTCTGAGGACGGACTCTTGACTCTTGAGTACTATTTTGTTCCAAATAGGTCCATTGCC
GAAACAAGAGTGCAAGAATTTGTTCAACATCCGAGGATGCAAATTCTGCTTAGCCATTCTGGATAGCCCATATGCTCTTAGGATC
CATCAAGACAGATGCCAGCTCTCGGGAGTAAATTATGTATGAACCCTTAGCTTGAAATTAAAACTACATATTTCAACTATATTTCC
TTCCCCCTATTCGCAAATTAAGTTTACACTGAAATTTTTACATGATATCTCCTTGTAGGGAATAGCGGCTCGTCTGGCTAACTTAG
GTCTTCGTGATAGCTTGACAATCGACAATGGTTTTTCAAGAGGCCCGCAAGTAGTTGCACTAGCTTGCAAAATGGTTGGTGCTG
GCAGTGACGGCTCATTAGATCTTTGTGCAAGGGTTTGCATCATTGATGAAAATGAGAATATGATCTACCATACTTATGTCAAACC
ACCAATTCCAGTTACAAACTACAGGTACCATTAATGAAAATGAGAATATGACTATGCCAATATGCGAATTCTGAGAATTCCAACAA
ATGGAATCAAACAAACCAATTAGGTTTTCTTCAACTACTTTCAGGTATGAAACCACAGGCATTCGACCAGAATATTTGAGAGATG
CAACGCCATTGAGACAAGTGCAAAGAAGGATTCAAGACTTCCTTTTCAATGGAGAACCAATGTGGAAAATTCGATCACCTAAAG
GTGGAAAAGCCAGGATTCTCGTGGGTCATGGTCTTGATCATGACCTTGACCGTCTCCAAGTAGAATACCCAGCAATCATGATAA
GGTAAAGTTTAAGGATGCTAGCATACATTCAAAAGGAACAGGTGCTGCTGCAATTATTTTATAGTCTAAATTGCACATTCATCTTT
TCCCTATATATTCTACTTCTAGAGACACAGCTCAATGATGAAATTCATGTGGAAAATACAATTAATATGGTATAAGTTTATGTGCC
TACAGGGATACTGCAAAATATCCTCCGTTAATGAAAACAAGCAAACTTAGCAACTCACTCAAGTACTTAACCCAAGCATACCTGG
GGTAAGCGTCTATACACAATATCTATTTAATTTCCTCATGATCATATCTGTCGATAATTATTCAATGATGCAACTTCTACATTCAGG
TATGACATCCAAACCGGCATTCAAGACCCTTATGAGGACTGCGTTGCCACAATGAGGCTTTACATGAGGATGAGGAATCAAGCT
CATAGGAGAGAAAACTATCCACTGGCTTCTGACCCTCAAAACCGAAACAACTTTGCATCATGGAGGCAAAGTGAGCTCGAGAG
GATGTCCCCAGAAGAAATGCTGGCTACCTCAAGGTCTGACTACTACTGTTGGTGTTTGGACTCTGAATGAGCATTGAACGCCAG
CATATATATGTGTAGTTCTCATGTGGTCAAACAGGTGTTCCATTCTGATTAAAGTTTAAGGATGCTA
SEQIDNO: 355
MASEPDPSESRTQRHKCPACYKQFKRKLHLIEHMKISYHSVHQPRCGVCQKHCKSFESVREHISGPLAKSNCSKIFLNQGCNLCLKV
LDSPAALNEHKQKCCLNAPVPLGTKIMPCIESHVILSGSIMDERHNGKGHGAIAIDCEMVGGGSDGSIDLCARVCLVDEDENLIFHTYV
QPQIPVSNYRYEVTGVTEDHLRDAMPLNEVQDKILKILYNGESVGRIRLDGEKVRLLVGHGIQHDLDCLRMKYPGQLLRDTAKYRPLM
KTNLLSHSLKHLTKTYLGYDIQSGIHDPYEDCISVMRLYKRMRGQDHQVEGLGNERANSGFDSIRSTELEKLTPDELYEISISDYKCWC
LDLSKECSLGS
SEQIDNO: 356
CTTTGGAAAATGCTTCACGCCAAAACAATAAACACCCCACGCCCATACACACCGGAAAATTCCCCGTCATTTTCATCTCCGGATC
TAGTACCCCATGGCCTCCGAACCTGATCCTTCAGAATCCCGAACCCAAAGGCACAAATGCCCTGCATGCTATAAACAATTTAAG
AGAAAGCTGCATCTTATAGAGCATATGAAAATCTCATACCATTCAGTTCATCAACCTAGATGCGGGGTGTGCCAAAAGCACTGC
AAATCTTTTGAATCAGTGCGAGAACATATTAGTGGTCCATTGGCAAAGTCAAATTGTTCGAAGATTTTTTTGAACCAAGGTTGTAA
TCTTTGTTTAAAAGTTCTGGATAGCCCTGCTGCCCTTAATGAACATAAACAAAAGTGCTGCCTAAATGCTCCTGTTCCCCTTGGA
ACAAAGATCATGCCTTGTATAGAATCTCATGTTATTTTGTCAGGTTCAATCATGGACGAAAGGCACAATGGTAAAGGTCATGGAG
CCATTGCTATTGATTGTGAAATGGTTGGTGGTGGTAGTGATGGATCAATTGACCTTTGTGCTAGGGTATGTCTTGTTGATGAAGA
TGAGAATCTAATTTTCCACACATATGTTCAACCACAAATTCCAGTCAGTAATTATAGATATGAAGTAACTGGGGTAACAGAAGAC
CATTTGAGAGATGCCATGCCACTTAACGAAGTGCAAGATAAAATATTGAAAATTCTGTACAACGGAGAGTCTGTTGGAAGGATTC
GCCTAGATGGGGAAAAGGTTAGGCTTCTTGTGGGTCATGGGATACAGCATGATTTGGACTGCTTGAGAATGAAATATCCTGGCC
AGCTGCTGAGGGACACTGCAAAATACCGTCCGTTGATGAAAACAAATCTTCTTAGTCACTCACTGAAGCACCTTACCAAAACATA
TCTAGGGTATGATATCCAGTCAGGCATTCACGATCCGTATGAAGATTGTATCTCTGTGATGAGACTGTACAAGAGAATGCGTGG
CCAAGACCATCAGGTGGAAGGACTTGGAAATGAAAGAGCAAATAGCGGCTTCGATTCTATTAGATCCACAGAACTAGAGAAGCT
GACTCCAGATGAGCTATATGAGATCTCTATATCTGATTACAAGTGTTGGTGTCTAGACTTGAGCAAGGAATGCAGCCTTGGCAG
CTGATTTTCCTTTTTTTTGTTTTTTTTCAACTGAACATTTAGTCTTGGCAGGGCATGTTAAGAAGCGTCTGCCATTGGTGCTTTGT
GTCCCCTAGTGTGGGCGTCATTTGGGCTTAAAACTTTTCGAATTTGACAGGAGAGCAGTAAAAAAAAGAGAGGATAGAGGAGAG
AAGTGAAAACAAGCAAGTCACAATAGAAAGTCAACTTGTAACTGCTTGGATTGTATATATAACAAGAGAAATTATGGGGACGGTT
GATGTTTTACTAGATAATGTGATTGTAGGAAATATAATCATTGAGAATAGAACATTGCAACATTTGTAAAGGTCAATTGCTG
SEQIDNO: 357
CTTTGGAAAATGCTTCACGCCAAAACAATAAACACCCCACGCCCATACACACCGGAAAATTCCCCGTCATTTTCATCTCCGGATC
TAGTACCCCATGGCCTCCGAACCTGATCCTTCAGAATCCCGAACCCAAAGGTCCCATGTCTCTCTTTCTCTATATTTATGCTTGT
GCTCTTTCTGATTGTTGGTCATTTATTGATGAGACATGTTTGGATGCCTAGAAAATATGGGAAAGAATACGGACGAAAATGCGTG
TCATTGGTTCATTTACTTTGTTTTTAGTTGATCAACTCCTGGGTACTAAAAAGATAGTAACAAATTTATTTGTTTCCTCGCAGTTTC
TCAGAATCTCTCTGCTTTCCCCTCTTACAGTTTTGTTTGTTTCCATATGAGGTTTTAGCTGATTTCAATAATTTTTCTGGGATATTA
AATGCTACTTTACGTTTTACTTAATTTTGATCACTTATGTTGTTGCTGTTTCAAGTTAGCGTCTGATCGATCACACTTTTACGTTTT
GCTCTTTTAGCAAAAAAAAAAAAAAAAAGAAAATTGAATATGAGGCACAATACAAATAGTAAAGGGTAAAATGTAAAAGTATCAAT
CAAACGAAGTCTTAGGACCAAGATTTGGTAGACATAGAGAATATACATCAGCTATCATAATGTAGGCAATTTGAGTTTAACTGTT
GAAGTATGATACTGTGAAATACGGGAAAAAATGGATATTGCTGAGAAGTTTCCACTTTTGCTGCTTCAGACATAAACTATGTGAT
AATAACTCTCTATTTAGATCTTTAAGCTGATATTGTCTTTGTTTTTCTTGATGATGATACATTGGTTGTGCTTGTAATGCATGTAAG
TGTAACGATCTTTTTTCCATTATTTTTGTTACAGGCACAAATGCCCTGCATGCTATAAACAATTTAAGAGAAAGCTGCATCTTATA
GAGCATATGAAAATCTCATACCATTCAGTTCATCAACCTAGATGCGGGGTGTGCCAAAAGCACTGCAAATCTTTTGAATCAGTGC
GAGAACATATTAGTGGTGAGATAATATGCATGTTCATTCTCATTTGTCAATTTTTATTGTGTAAGCATCTCATCTCTTTCCTTTTAC
ATAGGTCCATTGGCAAAGTCAAATTGTTCGAAGATTTTTTTGAACCAAGGTTGTAATCTTTGTTTAAAAGTTCTGGATAGCCCTGC
TGCCCTTAATGAACATAAACAAAAGTGCTGCCTAAATGCTCCTGTTCCCCTTGTAAGTAGCCCAGTGAAACTATTAAAATGCTAA
AATAGAATCTTATCAATGATGGAGCTTTCCCCCCTCCGCCCTTCTTTATTTATCTATAACAATATGTTATATATGCCCTTTGGACA
CATGTTTTACTATATTTCTCTTGTATTTTGTGTTTTGGATGCCTTATTTGTAAGTTATATGATAAAATGAATCATGAAAACAAGGAA
AAAGTACATTTTGGTTAAAGTTGAGTAACTGGTGCATAGTGACCTTTCTAATCACATTCATACTGCAGAATAACTTGTTTTGGGTC
GTTATTTAATTCATTGCCAAATATAACCTAAATATTATGTTGACTGTTGCAGGGAACAAAGATCATGCCTTGTATAGAATCTCATG
TTATTTTGTCAGGTTCAATCATGGACGAAAGGCACAATGGTAAAGGTCATGGAGCCATTGCTATTGATTGTGAAATGGTTGGTG
GTGGTAGTGATGGATCAATTGACCTTTGTGCTAGGGTATGTCTTGTTGATGAAGATGAGAATCTAATTTTCCACACATATGTTCA
ACCACAAATTCCAGTCAGTAATTATAGGTACCATGCTCCGTATTTTATTTTATTTAATAAGGTCATTTTAAAGGAATAGTTCCTTTA
TCTCCATATATATGTTTCAGATATGAAGTAACTGGGGTAACAGAAGACCATTTGAGAGATGCCATGCCACTTAACGAAGTGCAAG
ATAAAATATTGAAAATTCTGTACAACGGAGAGTCTGTTGGAAGGATTCGCCTAGATGGGGAAAAGGTTAGGCTTCTTGTGGGTC
ATGGGATACAGCATGATTTGGACTGCTTGAGAATGAAATATCCTGGCCAGCTGCTGAGGTATATTGAACTATTTCTATCGCTGTA
AACACAGCAACCCATCTAAATTTCTTCTTGAGGAATCAGTTTGTTGAAGCTGCTGTCTGTAATTTTATTGGAACCCTATGTGATGT
CATTTCTTGTGTTTCCTCACTCATTCGATGGTATAACTTAGTTCACTTCTAGATGAAATATATCCAAGATATAACTACTTTAAGCGT
TAACAAAATTAACTCGTTCTGTTGTGTTTTGCATCTCATTTAATCCTATACTTTGTTTATTAATCACTTTTTAACTCTATCTAAGGAC
TTAATTGGCTTTGTAAGGAAAGGATGCATTGCATTGTCTAGTCAAACAGAGTGGAAAAATAAAGTGTAGAAGAATTGGGATGATT
TTAGATTGTCCTGCTGTTTTCACTTTCTGATTTGACATGGACTTCTTTCGACAACCCCTCCCCCCTTCCTGCCCTTTTTCTTCATT
TCTTTTTTCTTATTAAAGATGTTGTGCAGAAATGTCCTCCCTTACATGGGCTTTTCTATTGTGCAGGGACACTGCAAAATACCGTC
CGTTGATGAAAACAAATCTTCTTAGTCACTCACTGAAGCACCTTACCAAAACATATCTAGGGTAAGCTACTATTTCAGGATCTATT
ACACATTTTGGAGTTATTTCTTGCTCATTAAATTTGCTGCTGCTGCTCACTGTATCTTCTTTCTTGTTACACCCTTTTCCTCTAGCA
TCAACGTGCATTGTTTTACAGGTATGATATCCAGTCAGGCATTCACGATCCGTATGAAGATTGTATCTCTGTGATGAGACTGTAC
AAGAGAATGCGTGGCCAAGACCATCAGGTGGAAGGACTTGGAAATGAAAGAGCAAATAGCGGCTTCGATTCTATTAGATCCAC
AGAACTAGAGAAGCTGACTCCAGATGAGCTATATGAGATCTCTATATCTGATTACAAGTGTTGGTGTCTAGACTTGAGCAAGGAA
TGCAGCCTTGGCAGCTGATTTTCCTTTTTTTTGTTTTTTTTCAACTGAACATTTAGTCTTGGCAGGGCATGTTAAGAAGCGTCTGC
CATTGGTGCTTTGTGTCCCCTAGTGTGGGCGTCATTTGGGCTTAAAACTTTTCGAATTTGACAGGAGAGCAGTAAAAAAAAGAG
AGGATAGAGGAGAGAAGTGAAAACAAGCAAGTCACAATAGAAAGTCAACTTGTAACTGCTTGGATTGTATATATAACAAGAGAAA
TTATGGGGACGGTTGATGTTTTACTAGATAATGTGATTGTAGGAAATATAATCATTGAGAATAGAACATTGCAACATTTGTAAAGG
TCAATTGCTG
SEQIDNO: 358
MGSRHESSETLRNKCAACYRQFNKMEHLVEHMRTSYHSVHEPMCGICRKHCRSFESLREHLIGPLPKAECERIFKDRGCDLCLTVLS
SRSGLWNHREACQFSRSNNGVLCRLANLGIQDELRLDNSRAKVVALACKMVGGGSDGSLDLCARVCLIDEHERIIFHTYVKPQLPVT
NYRYETTGLRPEFLREAMPLKQVQRRIQDYLCNGEPIWHIRSRGGRARVLVGHGLDHDLKCLEMEDTAKYPPLMKTSKLSNSLKYLT
KAYLGYDIQTGQQDPYEDCVATLRLYMRMRSQNHDTEDCPLATDPQNRNNFASWRQNELERMTPEKLLEISRSDYYCWCLDSRTS
HTNS
SEQIDNO: 359
ATGGGCAGCAGGCACGAGTCTTCCGAGACTCTGAGGAACAAATGCGCTGCATGCTATAGGCAGTTCAACAAAATGGAACACCT
TGTGGAACACATGAGGACATCCTATCATTCGGTTCATGAACCCATGTGCGGGATTTGCAGGAAACATTGTCGATCATTTGAATCT
CTGAGAGAACATCTTATAGGGCCGCTCCCGAAAGCAGAATGTGAGAGGATTTTCAAGGACCGGGGATGTGACCTTTGCCTAAC
AGTCCTGAGCAGTCGCAGTGGGCTCTGGAATCACCGGGAAGCATGCCAATTCTCACGTTCGAACAATGGGGTTCTGTGTCGTC
TGGCAAATTTGGGCATACAAGATGAATTAAGGCTGGACAACAGTAGAGCGAAGGTGGTCGCCCTGGCTTGCAAAATGGTTGGA
GGCGGTAGCGATGGCTCTTTAGATCTGTGTGCAAGAGTTTGCCTCATCGATGAGCACGAAAGGATAATCTTTCATACATATGTC
AAGCCACAGCTTCCCGTCACAAACTACAGGTATGAAACAACGGGATTGCGACCAGAATTTCTAAGGGAAGCGATGCCGTTGAA
ACAAGTGCAGAGAAGGATTCAGGACTATTTATGCAACGGGGAACCAATATGGCACATACGTTCTAGAGGAGGAAGAGCTCGCG
TCCTTGTGGGACATGGTTTGGATCACGATCTCAAGTGTTTGGAAATGGAGGACACGGCCAAATATCCACCACTTATGAAAACCA
GCAAGCTTAGCAACTCGCTCAAGTATTTGACAAAAGCATACCTTGGCTATGACATTCAAACGGGCCAGCAAGATCCGTATGAAG
ACTGCGTTGCTACACTGAGGCTCTACATGAGAATGAGATCACAGAATCACGATACGGAGGACTGCCCTCTTGCTACCGACCCC
CAGAACCGCAACAATTTTGCATCATGGCGACAGAACGAGCTCGAGAGGATGACTCCTGAAAAATTGCTAGAGATCTCAAGGTCT
GACTATTATTGCTGGTGCTTGGACTCCAGAACCAGCCACACCAATTCCTGA
SEQIDNO: 360
ATGGGCAGCAGGCACGAGTCTTCCGAGACTCTGAGGTAAAATCTCTACCGTGCTATTGAATACTTGTTTTATTTATTGTTACCTC
CTGATCAAGATGAAAAGGTTCATGTTGTGCATGAAAAAGAGAGAGGCAATAATTTAGCGTTTCATGATGATGCTGTCTATAATGT
CTCAGGAACAAATGCGCTGCATGCTATAGGCAGTTCAACAAAATGGAACACCTTGTGGAACACATGAGGACATCCTATCATTCG
GTTCATGAACCCATGTGCGGGATTTGCAGGAAACATTGTCGATCATTTGAATCTCTGAGAGAACATCTTATAGGTTAATTAAATC
ATTTCTGTCCTAGCGAACACAGTAATTATTCAACCATTTTCGGCTCTCTGCCTTTTCCATTTGATCTGATGTGGTTGGTTGGTTCT
GACACATGCGGGATTGAATTGCAAAACCAGGGCCGCTCCCGAAAGCAGAATGTGAGAGGATTTTCAAGGACCGGGGATGTGA
CCTTTGCCTAACAGTCCTGAGCAGTCGCAGTGGGCTCTGGAATCACCGGGAAGCATGCCAATTCTCACGTTCGAACAATGTAT
GGAACGACAGTTAAACTCAGCTATATGTTCGCATTTCCATTCCACGACAAATAGTAAAACTTTTTTGTTTTTGTAGGGGGTTCTGT
GTCGTCTGGCAAATTTGGGCATACAAGATGAATTAAGGCTGGACAACAGTAGAGCGAAGGTGGTCGCCCTGGCTTGCAAAATG
GTTGGAGGCGGTAGCGATGGCTCTTTAGATCTGTGTGCAAGAGTTTGCCTCATCGATGAGCACGAAAGGATAATCTTTCATACA
TATGTCAAGCCACAGCTTCCCGTCACAAACTACAGGTAATGGCGCAACAAAAAGATCATCAAAATTCACATACTACACAGGCAC
ATAGACAGTGAATTTGCACTCTAGAGACGGACTTTGAAAAATTCTTCCGCAGGTATGAAACAACGGGATTGCGACCAGAATTTCT
AAGGGAAGCGATGCCGTTGAAACAAGTGCAGAGAAGGATTCAGGACTATTTATGCAACGGGGAACCAATATGGCACATACGTT
CTAGAGGAGGAAGAGCTCGCGTCCTTGTGGGACATGGTTTGGATCACGATCTCAAGTGTTTGGAAATGGAGTATCCACCGATA
ATGATCAGGCAAGTTAGCTAGTTACGGGGCCACCCACCAATTATATATATATATACACACTTTGCTGCAGGAACACCAATTTTGA
CACCTGTGAAACTCCTTTTTTTTCCTGAAAAAAAAAATTTTTTGGCAGGGACACGGCCAAATATCCACCACTTATGAAAACCAGC
AAGCTTAGCAACTCGCTCAAGTATTTGACAAAAGCATACCTTGGGTATGTAGCTTCATTAAACTCTGGATGTTAGTAGTACATTA
CATCAAGAATAACAACATCATGTTTTTTTCTTTTTGGGGGTGGCGGCGGTGGGCAGCTATGACATTCAAACGGGCCAGCAAGAT
CCGTATGAAGACTGCGTTGCTACACTGAGGCTCTACATGAGAATGAGATCACAGAATCACGATACGGAGGACTGCCCTCTTGCT
ACCGACCCCCAGAACCGCAACAATTTTGCATCATGGCGACAGAACGAGCTCGAGAGGATGACTCCTGAAAAATTGCTAGAGAT
CTCAAGGTCTGACTATTATTGCTGGTGCTTGGACTCCAGAACCAGCCACACCAATTCCTGA
SEQIDNO: 361
MEHLVEHMRTSYHSVHEPMCGICRKHCRSFESLREHLIGPLPKAECERIFKDRGCDLCLTVLSSRSALWNHREACQFSRSNNGVLC
RLANLGIQDELRLDNSRAKVVALACKMVGGGSDGSLDLCARICLIDEHERIIFHAYVKPQLPVTNYRYETTGLRPEFLREAMPLKQVQR
RIQDYLCNGEPIWHIRSRGGRARVLVGHGLDHDLKCLEMEDTAKYPPLMKTSKLSNSLKYLTKAYLGYDIQTGQQDPYEDCVATLRL
YMRMRSQNHDTEDCPLATDPQNRNNFASWRQNELERMTPEKLLEISRSDYYCWCLDSRTSHTNS
SEQIDNO: 362
ATGGAACACCTTGTGGAACACATGAGGACATCCTATCATTCGGTTCATGAACCCATGTGCGGAATTTGCAGGAAACATTGTCGA
TCATTTGAATCTCTGAGAGAACATCTTATAGGGCCGCTCCCGAAAGCAGAATGTGAGAGGATTTTCAAGGACCGGGGATGTGAC
CTTTGCTTAACAGTCCTGAGCAGTCGCAGTGCGCTCTGGAATCACCGGGAAGCATGCCAATTCTCACGTTCGAACAATGGGGT
TCTGTGTCGTCTGGCAAATTTGGGCATACAAGATGAATTAAGGCTGGACAACAGTAGAGCGAAGGTGGTCGCCCTGGCTTGCA
AAATGGTTGGAGGCGGTAGCGATGGTTCTTTAGATCTGTGTGCAAGAATTTGCCTCATCGATGAGCACGAAAGGATAATCTTTC
ATGCATATGTCAAGCCACAGCTTCCCGTCACAAACTACAGGTATGAAACAACGGGATTGCGACCAGAATTTCTAAGGGAAGCGA
TGCCGTTGAAACAAGTGCAGAGAAGGATTCAGGACTATTTATGCAACGGGGAACCAATATGGCACATCCGTTCTAGAGGAGGA
AGAGCTCGCGTCCTTGTGGGACATGGTTTGGATCACGATCTTAAGTGTTTGGAAATGGAGGACACGGCCAAATATCCACCACTT
ATGAAAACCAGCAAGCTTAGCAACTCGCTCAAGTATTTGACAAAAGCATACCTTGGCTATGACATTCAAACGGGCCAGCAAGAT
CCGTATGAAGACTGCGTTGCTACACTGAGGCTCTACATGAGAATGAGATCACAGAATCACGATACGGAGGACTGCCCTCTTGCT
ACCGACCCCCAGAACCGCAACAATTTTGCGTCATGGCGACAGAACGAGCTCGAGAGGATGACTCCTGAAAAATTGCTAGAGAT
CTCAAGGTCTGACTATTATTGCTGGTGCTTGGACTCCAGAACCAGCCACACCAATTCTTGA
SEQIDNO: 363
ATGGAACACCTTGTGGAACACATGAGGACATCCTATCATTCGGTTCATGAACCCATGTGCGGAATTTGCAGGAAACATTGTCGA
TCATTTGAATCTCTGAGAGAACATCTTATAGGTTAATTAAATCATTTCTGTCCTAGCCAACACAGTAATTATTCAACCATTTTCGGC
TCTCTGCCTTTTCCATTTGATCTGATGTGGTTGGTTCTGACACATGCGGGATTGAATTGCAAAACCAGGGCCGCTCCCGAAAGC
AGAATGTGAGAGGATTTTCAAGGACCGGGGATGTGACCTTTGCTTAACAGTCCTGAGCAGTCGCAGTGCGCTCTGGAATCACC
GGGAAGCATGCCAATTCTCACGTTCGAACAATGTATGGAACGACAATTAAACTCAGGTGTATATTCGCATTTCCATTCCACGACA
AATAGTAAAACTTTTTTGTTTTTTGTACGTAGGGGGTTCTGTGTCGTCTGGCAAATTTGGGCATACAAGATGAATTAAGGCTGGA
CAACAGTAGAGCGAAGGTGGTCGCCCTGGCTTGCAAAATGGTTGGAGGCGGTAGCGATGGTTCTTTAGATCTGTGTGCAAGAA
TTTGCCTCATCGATGAGCACGAAAGGATAATCTTTCATGCATATGTCAAGCCACAGCTTCCCGTCACAAACTACAGGTAATGGA
GCAACAAAAAGATCGTCAAAATTCACATACTACATAGGCACATAGAAAGTGAATTTGCACTCTAGCTAGAGACGGACTTTGAAAA
ATTCTTCTGCAGGTATGAAACAACGGGATTGCGACCAGAATTTCTAAGGGAAGCGATGCCGTTGAAACAAGTGCAGAGAAGGAT
TCAGGACTATTTATGCAACGGGGAACCAATATGGCACATCCGTTCTAGAGGAGGAAGAGCTCGCGTCCTTGTGGGACATGGTT
TGGATCACGATCTTAAGTGTTTGGAAATGGAGTATCCACCGATAATGATCAGGCAAGTTAGCTAGTTACGGGGCCGCCCACCAA
TTATATATATATACTTTGCTGCAGGAACACCAATTTTGACGCCTATGAAACTCCTTTTTTTTTTCTGAAAAAAAATTTTTTTGGCAG
GGACACGGCCAAATATCCACCACTTATGAAAACCAGCAAGCTTAGCAACTCGCTCAAGTATTTGACAAAAGCATACCTTGGGTA
AGTAGCTTAATTAAAGTCTGGATGTTAGTAGTACATTACATCAAGAATAACAACATCATGATTTTTTTTTTATTTTTTTTTTGGGGA
CTCTGCTAAGGAAAATGTTTTTTTCTTTTTGGTGGTGGTGGTGGGCAGCTATGACATTCAAACGGGCCAGCAAGATCCGTATGA
AGACTGCGTTGCTACACTGAGGCTCTACATGAGAATGAGATCACAGAATCACGATACGGAGGACTGCCCTCTTGCTACCGACC
CCCAGAACCGCAACAATTTTGCGTCATGGCGACAGAACGAGCTCGAGAGGATGACTCCTGAAAAATTGCTAGAGATCTCAAGG
TCTGACTATTATTGCTGGTGCTTGGACTCCAGAACCAGCCACACCAATTCTTGA
SEQIDNO: 364
MEHLVEHMRTSYHSVHEPMCGICRKHCRSFESLREHLIGPLPKAECERIFKDRGCDLCLTVLSSRSALWNHREACQFSRSNNGVLC
RLANLGIQDELRLDNSRAKVVALACKMVGGGSDGSLDLCARICLIDEHERIIFHAYVKPQLPVTNYRYETTGLRPEFLREAMPLKQVQR
RIQDYLCNGEPIWHIRSRGGRARVLVGHGLDHDLKCLEMEDTAKYPPLMKTSKLSNSLKYLTKAYLGYDIQTGQQDPYEDCVATLRL
YMRMRSQNHDTEDCPLATDPQNRNNFASWRQNELERMTPEKLLEISRSDYYCWCLDSRTSHTNS
SEQIDNO: 365
ATGGAACACCTTGTGGAACACATGAGGACATCCTATCATTCGGTTCATGAACCCATGTGCGGAATTTGCAGGAAACATTGTCGA
TCATTTGAATCTCTGAGAGAACATCTTATAGGGCCGCTCCCGAAAGCAGAATGTGAGAGGATTTTCAAGGACCGGGGATGTGAC
CTTTGCTTAACAGTCCTGAGCAGTCGCAGTGCGCTCTGGAATCACCGGGAAGCATGCCAATTCTCACGTTCGAACAATGGGGT
TCTGTGTCGTCTGGCAAATTTGGGCATACAAGATGAATTAAGGCTGGACAACAGTAGAGCGAAGGTGGTCGCCCTGGCTTGCA
AAATGGTTGGAGGCGGTAGCGATGGTTCTTTAGATCTGTGTGCAAGAATTTGCCTCATCGATGAGCACGAAAGGATAATCTTTC
ATGCATATGTCAAGCCACAGCTTCCCGTCACAAACTACAGGTATGAAACAACGGGATTGCGACCAGAATTTCTAAGGGAAGCGA
TGCCGTTGAAACAAGTGCAGAGAAGGATTCAGGACTATTTATGCAACGGGGAACCAATATGGCACATCCGTTCTAGAGGAGGA
AGAGCTCGCGTCCTTGTGGGACATGGTTTGGATCACGATCTTAAGTGTTTGGAAATGGAGGACACGGCCAAATATCCACCACTT
ATGAAAACCAGCAAGCTTAGCAACTCGCTCAAGTATTTGACAAAAGCATACCTTGGCTATGACATTCAAACGGGCCAGCAAGAT
CCGTATGAAGACTGCGTTGCTACACTGAGGCTCTACATGAGAATGAGATCACAGAATCACGATACGGAGGACTGCCCTCTTGCT
ACCGACCCCCAGAACCGCAACAATTTTGCGTCATGGCGACAGAACGAGCTCGAGAGGATGACTCCTGAAAAATTGCTAGAGAT
CTCAAGGTCTGACTATTATTGCTGGTGCTTGGACTCCAGAACCAGCCACACCAATTCTTGA
SEQIDNO: 366
ATGGAACACCTTGTGGAACACATGAGGACATCCTATCATTCGGTTCATGAACCCATGTGCGGAATTTGCAGGAAACATTGTCGA
TCATTTGAATCTCTGAGAGAACATCTTATAGGTTAATTAAATCATTTCTGTCCTAGCCAACACAGTAATTATTCAACCATTTTCGGC
TCTCTGCCTTTTCCATTTGATCTGATGTGGTTGGTTCTGACACATGCGGGATTGAATTGCAAAACCAGGGCCGCTCCCGAAAGC
AGAATGTGAGAGGATTTTCAAGGACCGGGGATGTGACCTTTGCTTAACAGTCCTGAGCAGTCGCAGTGCGCTCTGGAATCACC
GGGAAGCATGCCAATTCTCACGTTCGAACAATGTATGGAACGACAATTAAACTCAGTGTATATTCGCATTTCCATTCCACGACAA
ATAGTAAAACTTTTTTGTTTTTTGTACGTAGGGGGTTCTGTGTCGTCTGGCAAATTTGGGCATACAAGATGAATTAAGGCTGGAC
AACAGTAGAGCGAAGGTGGTCGCCCTGGCTTGCAAAATGGTTGGAGGCGGTAGCGATGGTTCTTTAGATCTGTGTGCAAGAAT
TTGCCTCATCGATGAGCACGAAAGGATAATCTTTCATGCATATGTCAAGCCACAGCTTCCCGTCACAAACTACAGGTAATGGAG
CAACAAAAAGATCGTCAAAATTCACATACTACATAGGCACATAGAAAGTGAATTTGCACTCTAGCTAGAGACGGACTTTGAAAAA
TTCTTCTGCAGGTATGAAACAACGGGATTGCGACCAGAATTTCTAAGGGAAGCGATGCCGTTGAAACAAGTGCAGAGAAGGATT
CAGGACTATTTATGCAACGGGGAACCAATATGGCACATCCGTTCTAGAGGAGGAAGAGCTCGCGTCCTTGTGGGACATGGTTT
GGATCACGATCTTAAGTGTTTGGAAATGGAGTATCCACCGATAATGATCAGGCAAGTTAGCTAGTTACGGGGCCGCCCACCAAT
TATATATATATACTTTGCTGCAGGAACACCAATTTTGACGCCTATGAAACTCCTTTTTTTTTTCTGAAAAAAAATTTTTTTGGCAGG
GACACGGCCAAATATCCACCACTTATGAAAACCAGCAAGCTTAGCAACTCGCTCAAGTATTTGACAAAAGCATACCTTGGGTAA
GTAGCTTAATTAAAGTCTGGATGTTAGTAGTACATTACATCAAGAATAACAACATCATGATTTTTTTTTTATTTTTTTTTGGGGACT
CTGCTAAGGAAAATGTTTTTTCTTTTTGGTGGTGGTGGTGGGCAGCTATGACATTCAAACGGGCCAGCAAGATCCGTATGAAGA
CTGCGTTGCTACACTGAGGCTCTACATGAGAATGAGATCACAGAATCACGATACGGAGGACTGCCCTCTTGCTACCGACCCCC
AGAACCGCAACAATTTTGCGTCATGGCGACAGAACGAGCTCGAGAGGATGACTCCTGAAAAATTGCTAGAGATCTCAAGGTCT
GACTATTATTGCTGGTGCTTGGACTCCAGAACCAGCCACACCAATTCTTGA
SEQIDNO: 367
MESSDTLRNKCAACYRQFNKMEHLVEHMRTSYHSVHEPTCGICKKHCRSFESLREHLIGPLPKAECERIFKERGCDLCLTTLSSRNA
LRVHRETCQLSRPSPGLLYRMANLGIYDDLKIDNSRTKVAALACKMVGGGSDGTLDLCARVSLIDEYENIIFHTYVKPHLPVTNYRYET
TGVRPEFLRDAIPLRQASRKIQDFLCNGEPLWKIRSRGGKATILIGHGLDHDLKCLEVEYPAIMIRDTAKYPPLMKTSKLSNSLKYLTKA
YLGYDIQTGIQDPYDDCVATMRLYMRMRSQVHKLEDYPLATDPQNRNNFAQWRQSELERMNPEEMLEISRSDFYCWCLDSKESF
SEQIDNO: 368
AGATACTCCAAAGGTCAGTCTTCTTATCTCTCAAGTAGGGCTCTGATTCACTAGCTACTTTAGTGGACAACGGAATGGAGTCTTC
AGACACTCTTCGGAACAAGTGCGCAGCATGCTATAGACAGTTCAACAAAATGGAGCATCTAGTGGAACACATGAGGACATCTTA
TCACTCAGTTCATGAACCCACATGTGGAATTTGCAAGAAACATTGCCGATCATTTGAATCTCTGAGGGAGCATCTCATAGGGCC
ATTGCCAAAGGCAGAATGTGAAAGGATATTCAAAGAACGAGGATGCGACCTTTGCTTAACCACCCTTAGCAGCCGAAATGCTCT
TAGGGTCCACCGAGAGACATGCCAACTCTCACGTCCGAGCCCTGGCCTACTTTATCGCATGGCTAACTTAGGCATCTATGATGA
CTTGAAGATTGACAACAGTAGAACAAAGGTTGCTGCACTTGCTTGCAAAATGGTTGGTGGTGGCAGTGATGGCACCTTAGATCT
TTGTGCAAGGGTTTCCCTAATTGATGAATATGAGAACATCATCTTTCACACCTATGTCAAGCCTCATCTTCCTGTCACGAACTATA
GGTATGAGACAACAGGCGTACGACCAGAATTCTTAAGAGATGCAATACCACTGAGGCAAGCATCAAGGAAGATCCAAGATTTTT
TATGCAATGGTGAACCATTATGGAAAATCCGATCTAGAGGTGGAAAAGCAACAATTCTCATAGGTCATGGCTTGGATCACGATC
TTAAATGCTTAGAAGTGGAGTACCCAGCAATAATGATCAGGGACACAGCGAAATATCCTCCACTGATGAAAACAAGCAAACTCA
GCAACTCTCTGAAGTACCTAACAAAAGCATACCTCGGGTATGACATTCAAACTGGTATACAAGATCCTTATGATGACTGTGTAGC
TACAATGAGGCTTTACATGAGAATGAGGTCCCAAGTTCATAAACTGGAGGACTACCCACTTGCTACTGACCCACAGAACCGTAA
CAATTTTGCACAATGGAGGCAAAGCGAACTTGAGAGAATGAATCCAGAGGAAATGTTGGAAATCTCAAGGTCTGATTTCTATTGT
TGGTGCTCGGACTCTAAAGAAAGTTTCTGAATATTGAAGAAGGCTAACATGTTCTAGGAGGCAGACATAAAATAGTGAGTCTAAA
ACTCCACGAGTGAGCAAACCTATAATTATGAAACAATAAACATGGAAATGTAGCTCATTTCCATTATGTATTAACTATGCGAAACA
AAATCCATGTACCTTGTTGTGATATTGTATGTGACTTTATAATGAATAATTTACCTTCACTTACTGAAG
SEQIDNO: 369
MTSVTRNKCAACYRQFNKMEHLVEHMRTSYHSVHEPTCGICKKHCRSFESLREHLIGPLPKAECERIFKERGCDLCLTTLSSRNALR
VHRETCQLSRPSPGLLYRMANLGIYDDLKIDNSRTKVVALACKMVGGGSDGTLDLCARVSLIDEYENIIFHTYVKPHLPVTNYRYETTG
VRPEFLRDAIPLRQASRKIQDFLCNGEPLWKIRSRGGKATILIGHGLDHDLKCLEVEYPAIMIRDTAKYPPLMKTSKLSNSLKYLTKAYL
GYDIQTGIQDPYDDCVATMRLYMRMRSQVHKLEDYPLATDPQNRNNFAQWRQSELERMNPEEMLEISRSDFYCWCSDSKESF
SEQIDNO: 370
ATGACTTCTGTAACCAGGAACAAGTGCGCAGCATGCTATAGACAGTTCAACAAAATGGAGCATCTAGTGGAACACATGAGGACA
TCTTATCACTCAGTTCATGAACCCACATGTGGAATTTGCAAGAAACATTGCCGATCATTTGAATCTCTGAGGGAGCATCTCATAG
GGCCATTGCCAAAGGCAGAATGTGAAAGGATATTCAAAGAACGAGGATGTGACCTTTGCTTAACCACCCTTAGCAGCCGAAATG
CTCTTAGGGTCCACCGAGAGACATGCCAACTCTCACGTCCGAGCCCTGGCCTACTTTATCGCATGGCTAACTTAGGCATCTATG
ATGACTTGAAGATTGACAACAGTAGAACAAAGGTTGTTGCACTTGCTTGCAAAATGGTTGGTGGTGGCAGTGATGGCACCTTAG
ATCTTTGTGCAAGGGTTTCCCTAATTGATGAATATGAGAACATCATCTTTCACACCTATGTCAAGCCTCATCTTCCTGTCACGAAC
TATAGGTATGAGACAACAGGCGTACGACCAGAATTCTTAAGAGATGCTATACCACTGAGGCAAGCATCAAGGAAGATCCAAGAT
TTTTTATGCAATGGTGAACCATTATGGAAAATCCGATCTAGAGGTGGAAAAGCAACAATTCTCATAGGTCATGGCTTGGATCACG
ATCTTAAATGCTTAGAAGTGGAGTACCCAGCAATAATGATCAGGGACACAGCGAAATATCCTCCACTGATGAAAACAAGCAAAC
TCAGCAACTCTCTGAAGTACCTAACAAAAGCATACCTCGGGTATGACATTCAAACTGGTATACAAGATCCTTATGATGACTGTGT
AGCTACAATGAGGCTTTACATGAGAATGAGGTCCCAAGTTCATAAACTGGAGGACTACCCACTTGCTACTGACCCACAGAACCG
TAACAATTTTGCACAATGGAGGCAAAGCGAACTTGAGAGAATGAATCCAGAGGAAATGTTGGAAATCTCAAGGTCTGATTTCTAT
TGTTGGTGCTCGGACTCTAAAGAAAGTTTCTGA
SEQIDNO: 371
ATGACTTCTGTAACCAGGAACAAGTGCGCAGCATGCTATAGACAGTTCAACAAAATGGAGCATCTAGTGGAACACATGAGGACA
TCTTATCACTCAGTTCATGAACCCACATGTGGAATTTGCAAGAAACATTGCCGATCATTTGAATCTCTGAGGGAGCATCTCATAG
GTAAACTTTTAATTCACAACCAGAGCTCTGCTGATATTTTTCTTCATAAAATTGATCTTGATTACAATGTTACTCATTTGCTTCTTC
AGGGCCATTGCCAAAGGCAGAATGTGAAAGGATATTCAAAGAACGAGGATGTGACCTTTGCTTAACCACCCTTAGCAGCCGAAA
TGCTCTTAGGGTCCACCGAGAGACATGCCAACTCTCACGTCCGAGCCCTGTAAAAATCCTCATACTTGCTATTGAGGCACAAAA
TACCCCCTTTTTTTTTCCTTCCTTTTCATTTTCCATAGTGATTCTAAAACCCACAACCTATTTTGATTTTTTCAGGGCCTACTTTATC
GCATGGCTAACTTAGGCATCTATGATGACTTGAAGATTGACAACAGTAGAACAAAGGTTGTTGCACTTGCTTGCAAAATGGTTG
GTGGTGGCAGTGATGGCACCTTAGATCTTTGTGCAAGGGTTTCCCTAATTGATGAATATGAGAACATCATCTTTCACACCTATGT
CAAGCCTCATCTTCCTGTCACGAACTATAGGTACTAATGAGTTTATATTGAGATTCCCAAGTAACTGCTGCTTACTAAATACCATA
CTCTAATCAAGGGTTAATTTTACTTAGGTATGAGACAACAGGCGTACGACCAGAATTCTTAAGAGATGCTATACCACTGAGGCAA
GCATCAAGGAAGATCCAAGATTTTTTATGCAATGGTGAACCATTATGGAAAATCCGATCTAGAGGTGGAAAAGCAACAATTCTCA
TAGGTCATGGCTTGGATCACGATCTTAAATGCTTAGAAGTGGAGTACCCAGCAATAATGATCAGGTAAGCTGAGTAAGGAAGCT
CGTGAGTCCTGACATCCAAGATTTCAAGCCATCAGCAGTCCAGTTTTTTAATAGCTATTTGGCATCACTTCGTATTCTATACAATT
GAAAGAATTGGACTGTAAAGATATAAACTCATTGCTACAGCCCCATGTTCAAGACTTAAATTAAAATTCAACGATTTTTTCTTTTCT
TTTTTTCTACAGGGACACAGCGAAATATCCTCCACTGATGAAAACAAGCAAACTCAGCAACTCTCTGAAGTACCTAACAAAAGCA
TACCTCGGGTAATTACCCTTCATGCACTATGATCCTACAGTCAAAAAATTATTAATATAGGTCTTTCAAGTAGTAACGAAAATTCT
GTTTTACAGGTATGACATTCAAACTGGTATACAAGATCCTTATGATGACTGTGTAGCTACAATGAGGCTTTACATGAGAATGAGG
TCCCAAGTTCATAAACTGGAGGACTACCCACTTGCTACTGACCCACAGAACCGTAACAATTTTGCACAATGGAGGCAAAGCGAA
CTTGAGAGAATGAATCCAGAGGAAATGTTGGAAATCTCAAGGTCTGATTTCTATTGTTGGTGCTCGGACTCTAAAGAAAGTTTCT
GA
SEQIDNO: 372
MEHLVEHMRTSYHSAHEPMCGICRKHCRSFESLREHLIGPLPKVECERIFKEQGCDLCLTIFSSRNALHVHREACQFSRGNNMGLTN
RMANLGIQDDLKMDSSRARVVALACKMVGGGSDGSLDLCARVCLIDESEKIIFHTYVKPRLPVTKYRYETTGMRPEFLRDAMPLRQV
QRKIQDYLCNGEPIWQIRSRAGKARILVGHGLDHDLKSLEVEDTADYPPLMKSSKLSNSLKYLTKTYLGYDIQTGIQDPYEDCVATMRL
YMRMRSQRHKVEDYPLAQDPQNRNNFAAFRQSELERMAPEKMLEISRSDYYCWCLDSSS
SEQIDNO: 373
ATGGAGCATTTAGTCGAACACATGAGGACGTCGTACCATTCAGCTCATGAGCCCATGTGTGGAATTTGTAGAAAGCATTGCCGA
TCGTTTGAGTCTCTACGGGAGCATCTTATAGGACCTTTACCAAAGGTTGAATGTGAAAGGATATTTAAGGAACAAGGATGTGATC
TTTGTTTAACCATCTTTAGTAGCCGTAACGCTCTCCATGTCCACCGAGAGGCATGCCAATTCTCACGTGGCAACAATATGGGTCT
GACAAATCGAATGGCAAACTTGGGCATCCAGGATGATTTGAAGATGGATAGTAGTAGAGCAAGAGTGGTTGCACTTGCTTGCAA
AATGGTAGGAGGTGGTAGTGATGGTTCCTTAGATCTTTGTGCAAGGGTTTGCCTCATCGATGAATCCGAAAAGATAATCTTTCAT
ACATATGTGAAGCCACGTCTTCCGGTCACGAAATATAGGTATGAAACAACAGGCATGAGACCAGAATTTTTACGGGATGCTATG
CCACTTAGGCAAGTGCAAAGAAAGATTCAAGATTACTTATGTAATGGGGAGCCCATATGGCAGATCCGCTCCAGAGCTGGAAAA
GCTAGGATCCTTGTAGGCCATGGATTAGATCATGATTTGAAAAGCCTTGAAGTAGAGGATACCGCAGATTACCCTCCACTGATG
AAATCAAGCAAGCTCAGCAACTCACTTAAATATCTAACGAAAACATACCTTGGATATGACATTCAAACTGGTATTCAAGATCCCTA
TGAAGACTGTGTTGCAACAATGAGGCTGTACATGAGAATGAGATCACAGCGTCATAAAGTGGAAGACTATCCGCTTGCTCAAGA
CCCACAAAATCGAAACAATTTTGCTGCATTTAGGCAAAGCGAGCTTGAGAGGATGGCTCCAGAAAAGATGCTGGAAATCTCAAG
GTCTGATTACTATTGCTGGTGCTTAGACTCTAGCAGCTAA
SEQIDNO: 374
ATGGAGCATTTAGTCGAACACATGAGGACGTCGTACCATTCAGCTCATGAGCCCATGTGTGGAATTTGTAGAAAGCATTGCCGA
TCGTTTGAGTCTCTACGGGAGCATCTTATAGGTATTCTTCAATCTCCTAGAAGCTATAACATGTTATGTGTCATTTAAGATTTGGC
TTCTAACGCGTGTTGATTTCCTATACAGGACCTTTACCAAAGGTTGAATGTGAAAGGATATTTAAGGAACAAGGATGTGATCTTT
GTTTAACCATCTTTAGTAGCCGTAACGCTCTCCATGTCCACCGAGAGGCATGCCAATTCTCACGTGGCAACAATATGGTAAAGA
TGCTCATTCTAACTATATTTTTTAAACTTTTCAGCATAAAATCTATACAAAATCAATCTTGTTGGATTTTCTTTTACAGGGTCTGACA
AATCGAATGGCAAACTTGGGCATCCAGGATGATTTGAAGATGGATAGTAGTAGAGCAAGAGTGGTTGCACTTGCTTGCAAAATG
GTAGGAGGTGGTAGTGATGGTTCCTTAGATCTTTGTGCAAGGGTTTGCCTCATCGATGAATCCGAAAAGATAATCTTTCATACAT
ATGTGAAGCCACGTCTTCCGGTCACGAAATATAGGTAATTAAATCTTTACAAAAGAACGGTAAATTATGTCACAACTATATTTCGA
CTAAACTTGAAATCTTGAAAACTTTTAGGTATGAAACAACAGGCATGAGACCAGAATTTTTACGGGATGCTATGCCACTTAGGCA
AGTGCAAAGAAAGATTCAAGATTACTTATGTAATGGGGAGCCCATATGGCAGATCCGCTCCAGAGCTGGAAAAGCTAGGATCCT
TGTAGGCCATGGATTAGATCATGATTTGAAAAGCCTTGAAGTAGAGTATCCGCAGATGATGAGGAGGCAAGTTAACCAGCATAT
AACTAAAGACTTTTATGTCATCAATCGTTACACTCAATGACTTAAATCTGAACCATCACACGAATAAAGACTTCTATGTCATAAATT
GTTGCACACAGTGACTGGTTGCGGTTTTCCACAATTTAAGAATTAAGCTGATCATTTTTGTTTGCTCAAGAACAAAGAAAAGAAA
AGTTGAAATGGTTTCACTAATCTGAGATTTTCTCTACAGGGATACCGCAGATTACCCTCCACTGATGAAATCAAGCAAGCTCAGC
AACTCACTTAAATATCTAACGAAAACATACCTTGGGTAGTAGCTTAGCCGAAATTTTTGTCACTTTTCAGTTTACTCATCACTCCT
GGGAGCATGACAAATTTTCCTTTTACAGATATGACATTCAAACTGGTATTCAAGATCCCTATGAAGACTGTGTTGCAACAATGAG
GCTGTACATGAGAATGAGATCACAGCGTCATAAAGTGGAAGACTATCCGCTTGCTCAAGACCCACAAAATCGAAACAATTTTGC
T
SEQIDNO: 375
MRTSYHSAHEPMCGICKKHCRSFESLREHLIGPLPKVECERIFKERGCDLCLTILSSRNALYVHREKCQFSRSNNIGLTHRMANLGIQ
DDLKIDNNRAKVVSLACKMVGGGSDGSLDLCARVCLIDEYEKIIFHTYVKPHLPVTNYRYETTGIRPEFLRDALPLWQVQKKIQDYLCN
GEPVWQIRSRCGKGRILVGHGLDHDLKSLEVEYPQMMDTAKYPPLMKTSKLSNSLKYLMKAYLGYDIQIGIQDPYEDCVATMRLYMR
MRSQRHKVEEYPLASEPQNRNNFAAWRQSELEKMSPEKMLDISRSDYHCWCLDSSS
SEQIDNO: 376
ATGAGGACGTCATATCATTCAGCACATGAACCCATGTGTGGAATTTGTAAAAAGCATTGTCGGTCATTTGAGTCTCTAAGGGAAC
ATCTTATAGGACCGTTACCCAAGGTAGAATGTGAAAGGATATTTAAGGAAAGAGGGTGTGATCTTTGTTTAACCATCCTTAGTAG
CCGTAACGCTCTCTACGTCCACCGAGAGAAATGCCAATTCTCACGCAGCAACAATATTGGTCTGACACATCGAATGGCAAACTT
AGGCATTCAGGATGATTTGAAGATCGATAATAATAGAGCAAAAGTGGTTTCACTTGCTTGCAAAATGGTCGGTGGAGGTAGTGA
TGGCTCCTTAGATCTTTGTGCAAGGGTTTGCCTCATTGATGAGTATGAGAAGATAATCTTTCATACATATGTGAAGCCACATCTG
CCTGTCACAAACTATAGGTATGAAACAACAGGCATAAGACCAGAATTTTTAAGGGATGCTCTGCCGCTTTGGCAAGTGCAAAAG
AAGATTCAAGATTACTTGTGTAATGGGGAGCCTGTATGGCAGATCCGCTCCAGATGTGGAAAAGGAAGGATCCTTGTAGGCCAT
GGATTAGATCACGATTTGAAAAGCCTAGAAGTCGAGTATCCACAGATGATGGACACAGCAAAATATCCTCCACTGATGAAAACG
AGCAAGCTGAGCAACTCGCTGAAATATTTAATGAAAGCATACCTTGGATATGACATTCAAATTGGTATTCAAGATCCCTATGAAG
ACTGTGTTGCAACAATGAGGCTGTACATGAGAATGAGATCACAGCGTCATAAAGTGGAAGAATATCCGCTTGCTTCAGAGCCAC
AAAATAGAAACAATTTTGCAGCATGGAGGCAAAGCGAGCTTGAGAAGATGTCTCCAGAAAAGATGTTGGATATCTCAAGGTCTG
ATTACCATTGCTGGTGCTTAGACTCTAGCAGCTAA
SEQIDNO: 377
ATGAGGACGTCATATCATTCAGCACATGAACCCATGTGTGGAATTTGTAAAAAGCATTGTCGGTCATTTGAGTCTCTAAGGGAAC
ATCTTATAGGTATTCTTCAAACTCCAAGAAGCTACATCATGTTCTTTGACATTTAAGATTTGGCTTCTTCTAACATCTTTGATTTTA
TATATAGGACCGTTACCCAAGGTAGAATGTGAAAGGATATTTAAGGAAAGAGGGTGTGATCTTTGTTTAACCATCCTTAGTAGCC
GTAACGCTCTCTACGTCCACCGAGAGAAATGCCAATTCTCACGCAGCAACAATATTGTACAGATGCTCAAACTAGAATATCTAAC
TACATTCTTTAACCTTTTCGGCATAGAATCAGGATAAAACAATTCTTGCTGGGTTTTCTTTTACAGGGTCTGACACATCGAATGGC
AAACTTAGGCATTCAGGATGATTTGAAGATCGATAATAATAGAGCAAAAGTGGTTTCACTTGCTTGCAAAATGGTCGGTGGAGGT
AGTGATGGCTCCTTAGATCTTTGTGCAAGGGTTTGCCTCATTGATGAGTATGAGAAGATAATCTTTCATACATATGTGAAGCCAC
ATCTGCCTGTCACAAACTATAGGTAATGAAATATTTACAAAACAATGGTAAATTATGTCACGAGTAAATTCTGACTGAACTTGAAA
TCTTAAAAACTTTTAGGTATGAAACAACAGGCATAAGACCAGAATTTTTAAGGGATGCTCTGCCGCTTTGGCAAGTGCAAAAGAA
GATTCAAGATTACTTGTGTAATGGGGAGCCTGTATGGCAGATCCGCTCCAGATGTGGAAAAGGAAGGATCCTTGTAGGCCATG
GATTAGATCACGATTTGAAAAGCCTAGAAGTCGAGTATCCACAGATGATGTTAAGGCAAGTTCTCTCAGCTGAACCAGCACATA
ACTAAAGACTTTTATGTCATAAATTGATACCCTCGATGACTTTAGTCTGAACCAGCACATGACTAAAGACTTCTATGTCATAAATT
GTTGCCCACAGTGACTAGTTCTGATTTTTCACAAATTCAATGTGATGCTGCTCATTTTTGTTTGCCATAAGAACAAAGAAAATAAA
AATTTTGAAATGGAGTCACTAGTCTTAGAATTCTTTCTACAGGGACACAGCAAAATATCCTCCACTGATGAAAACGAGCAAGCTG
AGCAACTCGCTGAAATATTTAATGAAAGCATACCTTGGGTAATTGCTTTGCTGCAATTTCTGTCACTTATCATTTTACTCATGATA
AACCTGGGAGCATGAATATTTTTTCTTTTACAGATATGACATTCAAATTGGTATTCAAGATCCCTATGAAGACTGTGTTGCAACAA
TGAGGCTGTACATGAGAATGAGATCACAGCGTCATAAAGTGGAAGAATATCCGCTTGCTTCAGAGCCACAAAATAGAAACAATT
TTGCAGCATGGAGGCAAAGCGAGCTTGAGAAGATGTCTCCAGAAAAGATGTTGGATATCTCAAGGTCTGATTACCATTGCTGGT
GCTTAGACTCTAGCAGCTAA
SEQIDNO: 378
MKISYHSVHQPQCGVCQKHCKSFASLREHSTGSLAKGNCSKISSERGCSLCMRIFDSPVSLCEHLEICCLPAPFPIDFVKMHWTESQI
DTNDSGRIPEAIAMDCEMVGGGSDGSLDICARVCLVDEEEKPIFHTYVVPLIPVTNFRNEVTGITEENMRDAMPLKEVQDKILQILYNG
ESIGRLRLHGGKARVLVGHSLGHDLNCLRMNYPDHLLRDTAKYPPLMKTNLVSHSLKYLTKTYLGYDIQSGLHDPYEDCVSAMRLYK
KMRSQCHPRKDMGMLLAAHNASTNTFDSWKPKELENMTPDELFNISKSNYKCWCLDSGPLS
SEQIDNO: 379
ATGAAAATCTCGTATCACTCAGTTCACCAGCCTCAATGTGGTGTGTGTCAAAAGCATTGCAAATCCTTTGCATCACTAAGGGAGC
ACTCGACTGGTTCACTGGCTAAAGGGAATTGTTCAAAGATATCCTCTGAAAGAGGTTGCAGCCTCTGTATGAGAATTTTCGACA
GTCCAGTTTCTCTCTGTGAACACCTGGAAATCTGTTGCCTACCTGCTCCTTTCCCCATTGATTTTGTGAAGATGCATTGGACTGA
ATCTCAAATTGATACTAATGACAGTGGGAGGATTCCTGAAGCAATCGCTATGGATTGCGAAATGGTTGGTGGAGGAAGTGATGG
GTCACTGGATATATGTGCTAGGGTGTGCCTTGTTGATGAAGAGGAAAAACCGATTTTCCATACTTATGTAGTACCGCTAATTCCT
GTTACCAATTTTAGGAACGAAGTTACTGGTATTACTGAGGAGAATATGAGAGATGCTATGCCACTCAAGGAAGTGCAAGATAAAA
TTTTGCAGATTCTCTACAATGGAGAGTCGATTGGAAGATTACGGTTACATGGTGGCAAAGCTAGAGTTCTTGTGGGTCATTCCCT
TGGGCACGATTTAAATTGCTTGAGAATGAATTATCCTGATCATCTCCTGAGGGATACAGCAAAATACCCTCCCTTAATGAAGACA
AACCTCGTCAGCCACTCGCTCAAGTACCTCACCAAGACCTATCTCGGGTACGATATCCAGTCGGGGTTGCATGATCCTTACGAA
GATTGTGTATCAGCAATGAGGTTGTACAAGAAAATGCGGTCACAATGTCACCCAAGAAAAGATATGGGGATGTTATTGGCTGCT
CATAATGCATCTACCAACACTTTTGATTCTTGGAAGCCCAAAGAACTTGAGAATATGACACCAGATGAACTTTTCAATATCTCCAA
ATCAAATTACAAGTGTTGGTGTTTGGATTCAGGGCCTCTTTCGTGA
SEQIDNO: 380
ATGAAAATCTCGTATCACTCAGTTCACCAGCCTCAATGTGGTGTGTGTCAAAAGCATTGCAAATCCTTTGCATCACTAAGGGAGC
ACTCGACTGGTGATATACGATACTACAGTTTGGAAAATTTATATGTCAAAGGTTGACTCAGGTGCTCATACGTATAATCATTTTTT
CAAACCTTTAGGTTCACTGGCTAAAGGGAATTGTTCAAAGATATCCTCTGAAAGAGGTTGCAGCCTCTGTATGAGAATTTTCGAC
AGTCCAGTTTCTCTCTGTGAACACCTGGAAATCTGTTGCCTACCTGCTCCTTTCCCCATTGTGAGTTGACCTGGGAAACTTTCCC
TCCTTTTACCTTAAACACAAATTGATAGATTCATAAGCTTTTTTTTTTTTCAGGAAATATTATGGTTGTTGGAAAATGGACTTTGAA
TGTTACAAGCATGTGAACGTCAAAAATTTTGTTTTATTATACAGGATTTTGTGAAGATGCATTGGACTGAATCTCAAATTGATACT
AATGACAGTGGGAGGATTCCTGAAGCAATCGCTATGGATTGCGAAATGGTTGGTGGAGGAAGTGATGGGTCACTGGATATATG
TGCTAGGGTGTGCCTTGTTGATGAAGAGGAAAAACCGATTTTCCATACTTATGTAGTACCGCTAATTCCTGTTACCAATTTTAGG
TATCTGGGTTATGCATATGATCTATTTATTCTTTAAGTTTTTAATTAGGTTAATAGGCATGCTCATCAGTTATAATTCTATGTATATT
ATTGTTTTAGGAACGAAGTTACTGGTATTACTGAGGAGAATATGAGAGATGCTATGCCACTCAAGGAAGTGCAAGATAAAATTTT
GCAGATTCTCTACAATGGAGAGTCGATTGGAAGATTACGGTTACATGGTGGCAAAGCTAGAGTTCTTGTGGGTCATTCCCTTGG
GCACGATTTAAATTGCTTGAGAATGAATTATCCTGATCATCTCCTGAGGTGAAGTCATTTTGGGGCAATTAAGGTTACAAATCCA
TTTTCATGAGATTACATTGCATCCAACAACCTGTTCGAAGAAATTGATTTATTGTCTTCAATGAGAATGGACTTCTTGTGTGGAGG
CTCGTGGACAATACACAGAGCTCATTATATTTTGGTTTATGGTGATTTATGTTCTTTAATTCCAAATCCTACTCCCTTTCCTTTGTC
CCATCTCCAAAAAAAGGGAGGAAATGAGCACCAGAAAATGGGGAGGATCGATCTTAAAAGGCTAACTTATTGGTTTATAAATTTT
AATGATTTCATATTTTCTTGAATTAACTTGACTGAGTTCTCGCCAGTGGATTGTAATATGAACTTCCATTTCTACCTAGTGTATCTA
CGAGCTGGTCATTGTATACTGATTGTCCTTGAATTTTAGCTTCTATAATCAAGAGTAATGAAATAAGAAAGATGTAGATGAAATTG
TTTTGCTGCGTACTGCGTTGCGCTTAATTTTTCACCCTCTTTATGTGCAGGGATACAGCAAAATACCCTCCCTTAATGAAGACAA
ACCTCGTCAGCCACTCGCTCAAGTACCTCACCAAGACCTATCTCGGGTAAGTTGCCATCCAGTGATTATTCTTGGGTTCCTTTTT
TGCTAATTTAAGATACAATTTACCAAAGGAATAGAAGAGCTTTGGAGCTAAATGGAAGTACCAGAGACCAAGTATATAAAAACAA
CCTGAACGACTCTTAGGGGTGGGCGTGGACATTTACAAGGGATTTTTGGCATTCCCTCTGAATAGTCCCCGGTTTTCGTGGGCT
TCCTATTAGTTGCTATTCATAAGTATCTGTCATTCCCTCTGAATAGTCCTCGATTTTTCTTTGGCTTCCTTTCAGTTACTAATCCAT
AATCTCCAATTTTTTTGTCATGAACTCTAGACCAAGTGAAAAATATATTGAAGTTAATAATGTTCTCTGTGTAACTGCAAGAATGG
CAGCTTCAGACAAGCAATATTTTTACAGTGAACTTTTCTGAAAATTGACAGTCTGTTTCTCATGATCATTGATCTGAATTGAGATTT
CAGTAAAATGGCCCAACAGTTTTGTGGTCAAATAGGAAAAATGGTTTTCATCTTTAAGTGCAATCTCCATATACTGCTAAAATAGT
TTAGTAGGGTAACTATTCTAGTCGATTTGCAATTCCTTTAAATGTTTTGATCATGAATATTGTAAGTTTGGTTGATGACATGGGCT
TTGGTTTAATAGGTACGATATCCAGTCGGGGTTGCATGATCCTTACGAAGATTGTGTATCAGCAATGAGGTTGTACAAGAAAATG
CGGTCACAATGTCACCCAAGAAAAGATATGGGGATGTTATTGGCTGCTCATAATGCATCTACCAACACTTTTGATTCTTGGAAGC
CCAAAGAACTTGAGAATATGACACCAGATGAACTTTTCAATATCTCCAAATCAAATTACAAGTGTTGGTGTTTGGATTCAGGGCC
TCTTTCGTGA
SEQIDNO: 381
MKISYHSVHQPQCGVCQKHCKSFASLREHSTGSLAKGNCSKISSERGCSLCMRIFDSPVSLCEHLEICCLPAPFPIDFVKMHWTESQI
DTNDSGRIPEAIAMDCEMVGGGSDGSLDICARVCLVDEEEKPIFHTYVVPLIPVTNFRNEVTGITEENMRDAMPLKEVQDKILQILYNG
ESIGRLRLHGGKARVLVGHSLGHDLNCLRMNYPDHLLRDTAKYPPLMKTNLVSHSLKYLTKTSLGYDIQSGLHDPYEDCVSAMRLYK
KMRSQCHPRKDMGMLLAAHNASTNTFDSWKPKELENMTPDELFNISKSNYKCWCLDSGPLS
SEQIDNO: 382
ATGAAAATCTCGTATCACTCAGTTCACCAGCCTCAATGTGGTGTGTGTCAAAAGCATTGCAAATCCTTTGCATCACTAAGGGAGC
ACTCGACTGGTTCACTGGCTAAAGGGAATTGTTCAAAGATATCCTCTGAAAGAGGTTGCAGCCTCTGTATGAGAATTTTCGACA
GTCCAGTTTCTCTCTGTGAACACCTGGAAATCTGTTGCCTACCTGCTCCTTTCCCCATTGATTTTGTGAAGATGCATTGGACTGA
ATCTCAAATTGATACTAATGACAGTGGGAGGATTCCTGAAGCAATCGCTATGGATTGCGAAATGGTTGGTGGAGGAAGTGATGG
GTCACTGGATATATGTGCTAGGGTGTGCCTTGTTGATGAAGAGGAAAAACCGATTTTCCATACTTATGTAGTACCGCTAATTCCT
GTTACCAATTTTAGGAACGAAGTTACTGGTATTACTGAGGAGAATATGAGAGATGCTATGCCACTCAAGGAAGTGCAAGATAAAA
TTTTGCAGATTCTCTACAATGGAGAGTCGATTGGAAGATTACGGTTACATGGTGGCAAAGCTAGAGTTCTTGTGGGTCATTCCCT
TGGGCACGATTTAAATTGCTTGAGAATGAATTATCCTGATCATCTCCTGAGGGATACAGCAAAATACCCTCCCTTAATGAAGACA
AACCTCGTCAGCCACTCGCTCAAGTACCTCACCAAGACCTCTCTTGGGTACGATATCCAGTCGGGGTTGCATGATCCTTACGAA
GATTGTGTATCAGCAATGAGGTTGTACAAGAAAATGCGGTCACAATGTCACCCAAGAAAAGATATGGGGATGTTATTGGCTGCT
CATAATGCATCTACCAACACTTTTGATTCTTGGAAGCCCAAAGAACTTGAGAATATGACACCAGATGAACTTTTCAATATCTCCAA
ATCAAATTACAAGTGTTGGTGTTTGGATTCAGGGCCTCTTTCGTGA
SEQIDNO: 383
ATGAAAATCTCGTATCACTCAGTTCACCAGCCTCAATGTGGTGTGTGTCAAAAGCATTGCAAATCCTTTGCATCACTAAGGGAGC
ACTCGACTGGTGATATACGATACTACAGTTTGGAAAATTTATATGTCAAAGGTTGACTCAGGTGCTCATACGTATAATCATTTTTT
CAAACCTTTAGGTTCACTGGCTAAAGGGAATTGTTCAAAGATATCCTCTGAAAGAGGTTGCAGCCTCTGTATGAGAATTTTCGAC
AGTCCAGTTTCTCTCTGTGAACACCTGGAAATCTGTTGCCTACCTGCTCCTTTCCCCATTGTGAGTTGACCTGGGAAACTTTCCC
TCCTTTTACCTTAAACACAAATTCATAGATTCATAAGCTTTTTTTTTTTTTCAGGAAATATTATGGTTGTTGGAAAATGGACTTTGAA
TGTTACAAGCATGTGAACGTCAAAAGTTTTGTTTTATTATACAGGATTTTGTGAAGATGCATTGGACTGAATCTCAAATTGATACT
AATGACAGTGGGAGGATTCCTGAAGCAATCGCTATGGATTGCGAAATGGTTGGTGGAGGAAGTGATGGGTCACTGGATATATG
TGCTAGGGTGTGCCTTGTTGATGAAGAGGAAAAACCGATTTTCCATACTTATGTAGTACCGCTAATTCCTGTTACCAATTTTAGG
TATCTGGGTTATGCATATGATCTATTTATTCTTTAAGTTTTTAATTAGGTTAATAGGCATGCTCATCAGTTATAATTCTATGTATATT
ATTGTTTTAGGAACGAAGTTACTGGTATTACTGAGGAGAATATGAGAGATGCTATGCCACTCAAGGAAGTGCAAGATAAAATTTT
GCAGATTCTCTACAATGGAGAGTCGATTGGAAGATTACGGTTACATGGTGGCAAAGCTAGAGTTCTTGTGGGTCATTCCCTTGG
GCACGATTTAAATTGCTTGAGAATGAATTATCCTGATCATCTCCTGAGGTGAAGTCATTTTGGGGCAATTAAGGTTACAAATCCA
TTTTCATGAGATTACATTGCATCCAACAACCTGTTCGAAGAAATTGATTTATTGTCTTCAATGAGAATGGACTTCTTGTGTGGAGG
CTCGTGGACAATATACAGAGCTCATTATATTTTGGTTTATGGTGATTTATGTTCTTTAATTCCAAATCCTACTCCCTTTCCTTTGTC
CCATCTCCAAAAAAAGGGAGGAAATAAGCACCAGAAAATGGGGAGGATCGATCTTAAAAGGCTAACTTATTGGTTTATAAATTTT
AATGATTTCATATTTTCTTGAATTAACTTGACTGAGTTCTCGCCAGTGGATTGTAATATGAACTTCCATTTCTACCTAGTGTATCTG
CGAGCTGGTCATTGTATACTGATTGTCCTTGAATTTTAGCTTCTATAATCAAGAGTAATGAAATAAGAAAGATGTAGATGAAATTG
TTTTGCTGCGTACTCCGTTGCGCTTAATTTTTCACCCTCTTTATGTGCAGGGATACAGCAAAATACCCTCCCTTAATGAAGACAA
ACCTCGTCAGCCACTCGCTCAAGTACCTCACCAAGACCTCTCTTGGGTAAGTTGCCATCCAGTGATTATTCTTGGGTTCCTTTTT
TGCTAATTTAAGATACAATTTACCAAAGGAATAGAAGAGCTTTGGAGCTAAATGGAAGTACCAGAGACTAAGTATATAAAAACAA
CCTGAACGACTCTTAGGGGTGGGCGTGGACATTTACAAGGGATTTTTGGCATTCCCTCTGAATAGTCCCCGGTTTTCGTGGGCT
TCCTATTAGTTGCTATTCATAAGTATCTGTCATTCCCTCTGAATAGTCCTCGATTTTTCTTTGGCTTCCTTTCAGTTACTAATCCAT
AATCTCCAATTTTTTTGTCATGAACTCTAGACCAAGTGAAAAATATATTGAAGTTAATAATGTTCTCTGTGTAACTGCAAGAATGG
CAGCTTCAGACAAGCAATATTTTTACAGTGAACTTTTCTGAAAATTGACAGTCTGTTTCTCATGATCATTGATCTGAATTGAGATTT
CAGTAAGATGGCCCAACAGTTTTGTGGTCAAATAGGAAAAATGGTTTTCATCTTTAAGTGCAATCTCCATATACTGCTAAAATAGT
TTAGTAGGGTAACTATTCTAGTCGATTTGCAATTCCTTTAAATGTTTTGTTCATGAATATTGTAAGTTTGGTTGATGACATGGGCTT
TGGTTTAATAGGTACGATATCCAGTCGGGGTTGCATGATCCTTACGAAGATTGTGTATCAGCAATGAGGTTGTACAAGAAAATG
CGGTCACAATGTCACCCAAGAAAAGATATGGGGATGTTATTGGCTGCTCATAATGCATCTACCAACACTTTTGATTCTTGGAAGC
CCAAAGAACTTGAGAATATGACACCAGATGAACTTTTCAATATCTCCAAATCAAATTACAAGTGTTGGTGTTTGGATTCAGGGCC
TCTTTCGTGA
SEQIDNO: 384
MESRNESSEISRNKCAACFRQFNKMEHLVEHMRISYHSVHEPMCGICKKHCRSYESLREHVIGPLPKQECKNIFSIRGCKFCLTILDSP
NARRVHQERCQLSGGLLASFANLGLRDNLTIDNGYARGRQVVALACKMVGGGSDGSIDLCARVCLIDENENIIFHTYVKPPIPVTNYR
YETTGIRPEYLRDATPLRQVQKKIQDFLCNGEPTWKIRPRGGKARILVGHDLDRLQLEYPAVMMRDTAKYPPLMKTSKLSNSLKYLTQ
AYLGYDIQTGMQDPYEDCVATMRLYIRMRSQNHTIEDYPLAFDPQNRNNFASWRQSELERMSPEEMFAISRSDYYCWCLDS
SEQIDNO: 385
CGTCTTAACTAGTTTTCTACTACTCTTGCAGATTTAACAACTATTTACTTTACGAGGATCGCATGGAGTCCAGAAATGAGTCGTCC
GAGATTTCAAGGAACAAGTGTGCTGCATGCTTTAGACAATTCAACAAAATGGAACACCTAGTGGAGCACATGAGAATATCGTAT
CACTCAGTCCATGAACCCATGTGTGGAATCTGTAAGAAACATTGCAGATCTTATGAGTCCTTGAGGGAGCATGTTATAGGGCCA
TTGCCTAAGCAAGAATGTAAGAATATTTTCAGTATCCGTGGATGCAAATTTTGCTTAACCATCCTTGATAGCCCTAATGCTCGCA
GAGTTCACCAAGAAAGATGCCAGCTCTCCGGTGGACTACTTGCAAGCTTTGCTAATTTAGGCCTTCGGGACAACCTGACAATCG
ACAATGGTTATGCAAGAGGTCGACAGGTAGTTGCCCTAGCTTGCAAAATGGTTGGAGGAGGCAGTGATGGCTCCATAGACCTT
TGCGCAAGGGTTTGCCTGATTGACGAAAATGAAAATATTATCTTCCATACCTATGTCAAACCACCAATTCCTGTCACAAACTATA
GGTATGAAACCACAGGCATTAGACCAGAATACCTAAGGGATGCAACACCATTAAGGCAAGTTCAAAAGAAGATTCAAGACTTCC
TTTGTAATGGAGAACCGACGTGGAAAATTCGTCCTAGAGGAGGAAAAGCCAGGATTCTCGTGGGTCATGACCTTGACCGTTTG
CAACTAGAATATCCAGCTGTAATGATGAGGGATACTGCAAAATATCCGCCACTGATGAAAACAAGCAAGCTCAGCAACTCCTTG
AAGTATCTAACCCAAGCCTATCTCGGGTATGACATCCAAACTGGCATGCAAGATCCTTATGAAGATTGTGTTGCTACAATGAGG
CTCTACATAAGAATGAGATCACAAAATCATACGATAGAGGATTACCCTCTCGCTTTTGATCCACAAAACCGAAACAATTTTGCTTC
GTGGAGGCAAAGTGAGCTTGAAAGGATGAGCCCCGAAGAAATGTTTGCCATATCAAGATCTGATTACTACTGCTGGTGCTTGGA
TTCTTAAGCAAGGGACTTTTCCCGACTAGTAAGCAAAGGCAACTTTGTGTCCATGCGAAATGAACACACTTCCAAACTATTCGAT
ATTTTATATTAAGAAGAAAAATAAATTGGGAAGCTTCTGCCATTCCATTACCACTCCCCTCTAGATTACTGGAACGTACTGAATAT
GTTATTCAATATGTAATAAGTAGTTTCTTTAATATAAATTTGGTAATATATATTTAATAAAAGTATAACACATAATGGTAATTTATGT
GTTAATTACTGCAATTTCTGTGTCTAA
SEQIDNO: 386
CGTCTTAACTAGTTTTCTACTACTCTTGCAGATTTAACAACTATTTACTTTACGAGGATCGCATGGAGTCCAGAAATGAGTCGTCC
GAGATTTCAAGGTTCTCACTCTCACTCTCTCCTCTCCACGCACATATACATGGAAACCGACACGTACATGGTTTTGATTCAAGCT
ATATCATGCATGTCATATTGTCAAATTGGTGACCTTCATGTCAAGCTAGCTTTTAATTCCTAAACCCTAGTTTCTAGCTAGCGATA
AATAACTTGATGATTATCCTTCTGCAGGAACAAGTGTGCTGCATGCTTTAGACAATTCAACAAAATGGAACACCTAGTGGAGCAC
ATGAGAATATCGTATCACTCAGTCCATGAACCCATGTGTGGAATCTGTAAGAAACATTGCAGATCTTATGAGTCCTTGAGGGAG
CATGTTATAGGTAAATGTTTGTTTTCTTAGCGTGTACAACTCGGAGCACTTGGTTTTCTCTTCGGTAGCTTAAAAGTTTGATTTTC
TCTTACAGGGCCATTGCCTAAGCAAGAATGTAAGAATATTTTCAGTATCCGTGGATGCAAATTTTGCTTAACCATCCTTGATAGC
CCTAATGCTCGCAGAGTTCACCAAGAAAGATGCCAGCTCTCCGGTGTAAATGCTGTATTAATCTTTAATTTAATACTTCGAAAAA
AAAGATAAACTAAATTAAGTTTTGTTTTTTCAACCGAATTAGTTCTAAACTAAAAGAATGCCTCACTAGTTCTTTTATTGCACACTC
CAGGGACTACTTGCAAGCTTTGCTAATTTAGGCCTTCGGGACAACCTGACAATCGACAATGGTTATGCAAGAGGTCGACAGGTA
GTTGCCCTAGCTTGCAAAATGGTTGGAGGAGGCAGTGATGGCTCCATAGACCTTTGCGCAAGGGTTTGCCTGATTGACGAAAA
TGAAAATATTATOTTCCATACCTATGTCAAACCACCAATTCCTGTCACAAACTATAGGTATATATGCTATAAATTTATATACTGCTT
CTGCCCGAAAATCAAACAATGCAGTCGTTAATAGTGAATTAAATATTCTTCAAATATTTCTAGGTATGAAACCACAGGCATTAGAC
CAGAATACCTAAGGGATGCAACACCATTAAGGCAAGTTCAAAAGAAGATTCAAGACTTCCTTTGTAATGGAGAACCGACGTGGA
AAATTCGTCCTAGAGGAGGAAAAGCCAGGATTCTCGTGGGTCATGACCTTGACCGTTTGCAACTAGAATATCCAGCTGTAATGA
TGAGGTTAAGTTTTTCTCCGAAGCTTAGCTATACAAGTCATAGCCTCAAAAATAATTGCCTTGATCTACTTCTAATTATTTATCAGT
TTAAATTATAAAAATTCGTCTTTTTTAAAACAAAATATAATGTGATTAATTAAGAAGGATGGATTTCATTACAGGGATACTGCAAAA
TATCCGCCACTGATGAAAACAAGCAAGCTCAGCAACTCCTTGAAGTATCTAACCCAAGCCTATCTCGGGTAAGCATATGCATCTT
CTGCTATATTTGTTTTCCTTCCGTGTCTAAGTATATGTTTAATTTACTTGCAGGTATGACATCCAAACTGGCATGCAAGATCCTTA
TGAAGATTGTGTTGCTACAATGAGGCTCTACATAAGAATGAGATCACAAAATCATACGATAGAGGATTACCCTCTCGCTTTTGAT
CCACAAAACCGAAACAATTTTGCTTCGTGGAGGCAAAGTGAGCTTGAAAGGATGAGCCCCGAAGAAATGTTTGCCATATCAAGA
TCTGATTACTACTGCTGGTGCTTGGATTCTTAAGCAAGGGACTTTTCCCGACTAGTAAGCAAAGGCAACTTTGTGTCCATGCGAA
ATGAACACACTTCCAAACTATTCGATATTTTATATTAAGAAGAAAAATAAATTGGGAAGCTTCTGCCATTCCATTACCACTCCCCT
CTAGATTACTGGAACGTACTGAATATGTTATTCAATATGTAATAAGTAGTTTCTTTAATATAAATTTGGTAATATATATTTAATAAAA
GTATAACACATAATGGTAATTTATGTGTTAATTACTGCAATTTCTGTGTCTAA
SEQIDNO: 387
MDAEADPPTRSRTERHKCFACYKQFKKKEHLVEHMKISYHTPHQPKCGVCQKHCKSFESLRNHLTGPLSRAGCSRTFSAQGCDLCL
KLYDSPSSLGKHREICHLSAPASLGTKILPFAGSVDVKYTTKGAKAIAINCGLVGGGTDGSLDLCARVCLVDEDENIIFHTYVQPQSAVT
DYRYEITGLTEEHLRNSKSHKEVQDRILEILYNGESARRLMSDSGKARLLVGHDLKRGLDCLRINYPGHLLRDTAKYRPLLKTNLVSHS
LKYLTKTYLGYDIQTGEHDPYVDCVSVMRLYKRMRAQDHQGKGIGTPDSDSGFESQKAEELENMTPDELYQISKSDYKCWCLDSSIA
AGLNS
SEQIDNO: 388
GAAGACTAGTTTGATTTTTGCAACTTCATCAACCAGTTAAACAGTGAAGAAATGCACTCCGATTTGTTCATCTCTTTTTCTTTTCTT
TTTTAAATATACTACCATCAAAAACCCATCCTAACCCCTTCTCCTCCTCTGTCTCTGTGACGCCCGATCCTAAGAAGAATCGCCA
TGGATGCTGAAGCTGACCCACCAACAAGATCGCGAACCGAAAGGCACAAGTGCTTTGCATGCTACAAGCAATTCAAGAAAAAA
GAACATCTTGTTGAGCACATGAAGATTTCCTACCACACACCTCATCAGCCTAAATGTGGGGTCTGCCAAAAGCACTGCAAATCTT
TTGAATCTCTGAGGAATCACCTTACTGGCCCATTATCAAGGGCTGGTTGTTCGAGGACTTTCTCTGCCCAAGGTTGTGATCTCT
GCTTGAAACTCTATGATAGCCCCAGTTCTCTTGGCAAGCATAGAGAAATATGCCACCTATCTGCTCCTGCTTCCCTTGGAACAAA
GATATTGCCTTTTGCAGGATCTGTTGATGTAAAGTACACCACCAAAGGTGCCAAAGCCATTGCTATCAACTGTGGGTTGGTTGG
TGGTGGAACCGATGGATCACTTGACCTTTGCGCAAGGGTGTGTCTGGTTGATGAAGATGAAAATATTATTTTCCATACATATGTT
CAACCTCAAAGTGCTGTTACCGATTACAGATACGAAATAACTGGATTAACTGAAGAGCATCTAAGAAATTCGAAGTCTCATAAGG
AAGTACAAGACAGAATTCTGGAAATTTTGTACAATGGAGAATCTGCTAGGAGACTAATGTCAGACAGTGGAAAGGCTAGACTGC
TTGTGGGTCATGACTTGAAGCGTGGTTTGGATTGCTTGAGGATAAACTATCCTGGTCATTTGCTGAGGGATACTGCAAAATACC
GACCATTGTTGAAAACTAACCTGGTTAGCCACTCGCTCAAGTACCTCACCAAAACATATCTTGGGTATGATATCCAGACAGGGG
AACATGATCCATACGTAGATTGTGTTTCTGTTATGAGACTGTACAAGAGAATGCGTGCTCAAGATCATCAAGGTAAAGGAATTGG
AACTCCAGATTCAGACAGCGGTTTTGAGTCCCAGAAAGCAGAGGAACTCGAGAACATGACCCCAGATGAGCTATACCAGATCT
CAAAGTCAGACTACAAATGTTGGTGTCTAGATTCAAGTATAGCAGCAGGGCTTAACAGTTGATTTTTAGTTTCTCATCTTTGATCT
TTGTGCACTCGGCGGGACAATATTTTGCTGCTCAAAGAAATAGGAATGACGTGATGTGCTGCTAGGTATGAAGGATTCGTGTAC
AGTTTTTGCTGTAATGCAAAGTGAAGCATATATTCGCGTAGTTGTTTATATATGTTTGTTTACATTTCTTGAACTCTAAAGCAAACG
GGAGAGTTTCTAATATT
SEQIDNO: 389
GAAGACTAGTTTGATTTTTGCAACTTCATCAACCAGTTAAACAGTGAAGAAATGCACTCCGATTTGTTCATCTCTTTTTCTTTTCTT
TTTTAAATATACTACCATCAAAAACCCATCCTAACCCCTTCTCCTCCTCTGTCTCTGTGACGCCCGATCCTAAGAAGAATCGCCA
TGGATGCTGAAGCTGACCCACCAACAAGATCGCGAACCGAAAGGTCTCTCTCGCTCCCTCTTTCTCGCTGTATTCTGCATTTTTT
TTTTTTATTCTCGCTTTCATATGTGTCAATAATTTGTTCTTACAATTCGTTTCTTTTTCTTGATAATGAATTCGGGCTGTCTTCTTCA
GAAACTCCAAGTCCATGCCATGTATATGAAATTGTTTAATTCCTCGTGTTGTTTCATGGAAGCAGGCACAAGTGCTTTGCATGCT
ACAAGCAATTCAAGAAAAAAGAACATCTTGTTGAGCACATGAAGATTTCCTACCACACACCTCATCAGCCTAAATGTGGGGTCTG
CCAAAAGCACTGCAAATCTTTTGAATCTCTGAGGAATCACCTTACTGGTATCATTCTATTCCATGTTCTGATTGCTTTATCTGTTA
AAGTTTACTGCTTTTTTTTTCATGTTTTCCTCCAATTATTTAGGCCCATTATCAAGGGCTGGTTGTTCGAGGACTTTCTCTGCCCA
AGGTTGTGATCTCTGCTTGAAACTCTATGATAGCCCCAGTTCTCTTGGCAAGCATAGAGAAATATGCCACCTATCTGCTCCTGCT
TCCCTTGTAAGTTGTTGATTCGTAAATTCTTATGCAAAAATGGTAACTTGTCTATCTTTTAGTGCTTTTCATTTTATTTATTGTACAT
TTTAAATGATTAATTACTAAAAGTCATGTCACCCATTTCCTTTTTTTTTTCACAGGGAACAAAGATATTGCCTTTTGCAGGATCTGT
TGATGTAAAGTACACCACCAAAGGTGCCAAAGCCATTGCTATCAACTGTGGGTTGGTTGGTGGTGGAACCGATGGATCACTTGA
CCTTTGCGCAAGGGTGTGTCTGGTTGATGAAGATGAAAATATTATTTTCCATACATATGTTCAACCTCAAAGTGCTGTTACCGATT
ACAGGTACTTCTGTGTTGACACAATCCTTGGTTTTATTTAAAGAACAATGAGGGGGAGGGGAGGGGGAATATATTTACAAAAGT
CTTTTTTTTTTTAATTGAAACTTATACACATGTTTTCAGATACGAAATAACTGGATTAACTGAAGAGCATCTAAGAAATTCGAAGTC
TCATAAGGAAGTACAAGACAGAATTCTGGAAATTTTGTACAATGGAGAATCTGCTAGGAGACTAATGTCAGACAGTGGAAAGGC
TAGACTGCTTGTGGGTCATGACTTGAAGCGTGGTTTGGATTGCTTGAGGATAAACTATCCTGGTCATTTGCTGAGGTAGATTAAA
CTCTTCTGATGGTTGAAATTGGAATTTGGGATGGAACATTACAGTTTGTAATAATACTTATGATTCAGGCTTGAAAAACATTACTG
AGCCGCACCACTGCTTTACAAATCTGACCTTTGAATAATTGAATAATACCACTAATCCGCCATGTAGTCTACTTCTAGGTGAAGC
AAATCCTAAAATACTACTAATTGCTGATGGAAGAATGACACTTGGTGGTCATTTTGACGTGTCTTGGAATAAACCAACAAATTTTC
ATATTATTGAATAGGAGCTTTTTGCTGCACATTGAGTACTGATAATAATTGAAGGTTTGGTAAGCTTTCTTGTGACTTTACTGTTG
TTTTGCAACATTCTAGCTTTTCATGGGATGTATCCAAACACATTCTAAATGAACATAAGACCAGATAGTGGTGAGGTTTACCTCGT
CTTAAACTCCCTTCCCCATGCTAGGAGTGCCGTAGTATATCTTCAACTGCTGGCAAATCTTGTCTTGGCATCCATATTAGGAGTG
ACCCCCCCTACAGGCCAAGACAGAACCTTGGGGACTGGCCCAAATTATGGTAGGTAATTGAGAAAACAATTCAAATAGTTTTTTT
TTTTTCTTGTTCTGGAGATCCTGTAATTTGAAATGTAATATTTGAGTTATCCATCACAAATGTTGTGTGCATGTTCTCCTTGTTCTC
ATCTTTCCTTCTTTTATAGTTACAATGCACTTTGATTTTGATTTGAGCAGGGATACTGCAAAATACCGACCATTGTTGAAAACTAA
CCTGGTTAGCCACTCGCTCAAGTACCTCACCAAAACATATCTTGGGCAAGTTGCTCTTTCTTTTCTTGTTTCTTTCGTGATGTCTT
AATAGTGATTGGATCTTCCCTCCTGCATGTACGATGGCTGAATCCTTATATGGCTAACTATTTTAATTAGTAACCAATACTTGAAC
TAGAAATGATGCTTTTGTGTTATCTGTCTTTTTTTTTTTTTTCCAAGTAAAATGCAACTCAGATGTAATTAAAAATTATGCAAATCAA
TTCCCAAGTTAAAAGATTTTTTTTTTTTGAAAATTTTATAACACTTGGATTTACAATGCTCGAACGCTGTATTTCTGTAAACATAGTT
GGTAACTATTTCTTCCTCAATTCTTAGCAGAAAAGACTGGAATTTCTCATCATGTTCGTCAAAGAGGATTTGGGAAAGATGATATC
TTCTTTGTGCCTAATATGTTGGTTTTCCAGAACTCAGATACTACTTTCAGCAAACACTGTTTTGAAAGTCATTATTGGTTCACAAAT
AAGAGGTCATTGTGGAGAGTTTAACGAACATTTAACCTTTTGTTGATCTGTTTTTCTAGTTTTTGGAATGATCACCGTGCACTTCA
TATTTATGGGCAGTATTTGCTAGTTGAAATTACTCTCTCTGAAGTTTTTTGCCTCACAGGTATGATATCCAGACAGGGGAACATG
ATCCATACGTAGATTGTGTTTCTGTTATGAGACTGTACAAGAGAATGCGTGCTCAAGATCATCAAGGTAAAGGAATTGGAACTCC
AGATTCAGACAGCGGTTTTGAGTCCCAGAAAGCAGAGGAACTCGAGAACATGACCCCAGATGAGCTATACCAGATCTCAAAGT
CAGACTACAAATGTTGGTGTCTAGATTCAAGTATAGCAGCAGGGCTTAACAGTTGATTTTTAGTTTCTCATCTTTGATCTTTGTGC
ACTCGGCGGGACAATATTTTGCTGCTCAAAGAAATAGGAATGACGTGATGTGCTGCTAGGTATGAAGGATTCGTGTACAGTTTT
TGCTGTAATGCAAAGTGAAGCATATATTCGCGTAGTTGTTTATATATGTTTGTTTACATTTCTTGAACTCTAAAGCAAACGGGAGA
GTTTCTAATATT
SEQIDNO: 390
MDPSETLRNKCAACYRQFNRFEHLVEHMRTSYHSVHEPMCGICGKRCRSFESLREHLIGPLPKAECERVFRDRGCDICLTILTSRYAL
RAHRDACQLSCGNNGLLHRFANMGIQDDLRIDSGKSRAVALACKMVGGGSDGSLDLCAKICIIDEYENTIFRSYVKPQLPVTNYRYET
TGIRPEYLRDAMPLRQVQRKIQDFLCNGEPIWKIRPRGGKARILVGHGLDHYLRCLEFDYPSLKIRDTATYPPLMKTSKLSNSLKYLTK
AYLGYDIQVGIQDPYEDCVATMRLYRRMRSQAHRNENHPLASDPQNKNNFASWRQGELERMSPNDLLAISRSDYYCWCLDNKDYI
SEQIDNO: 391
ATGGATCCTTCAGAAACTCTAAGGAACAAGTGTGCAGCCTGCTATCGGCAATTCAACAGATTTGAGCACCTAGTGGAACACATG
AGAACTTCATATCACTCGGTTCATGAACCCATGTGTGGAATTTGTGGAAAACGATGCCGATCGTTTGAATCTCTTCGTGAGCATC
TTATAGGGCCATTGCCAAAGGCAGAATGTGAAAGGGTGTTCCGCGACCGTGGATGTGACATTTGCTTAACCATCCTTACCAGCC
GTTATGCTCTCAGGGCTCACCGTGATGCATGCCAACTCTCTTGTGGCAATAACGGTTTATTACACCGATTTGCTAACATGGGGA
TACAAGATGATTTAAGAATCGATAGCGGCAAATCAAGAGCAGTTGCACTTGCGTGCAAAATGGTCGGTGGTGGTAGTGATGGTT
CATTGGATCTTTGTGCAAAAATTTGCATCATTGATGAATATGAAAACACAATATTTCGTTCATATGTCAAACCGCAGCTTCCTGTT
ACAAATTACAGGTATGAGACAACAGGCATTCGGCCAGAATACCTTCGGGACGCAATGCCACTTAGGCAGGTTCAAAGAAAGATT
CAAGATTTTCTATGCAATGGTGAACCCATTTGGAAGATCCGCCCAAGAGGTGGGAAGGCAAGGATTCTTGTAGGTCATGGTTTG
GATCATTATCTTAGATGCTTGGAATTCGATTACCCTTCACTTAAAATTAGGGATACAGCAACCTATCCGCCATTAATGAAGACTAG
CAAGCTCAGCAATTCATTGAAGTATCTCACAAAAGCATACCTTGGATATGATATTCAAGTTGGGATACAAGATCCTTATGAAGAC
TGTGTTGCAACAATGAGACTTTACAGGAGGATGAGATCACAGGCTCATAGAAACGAGAACCATCCGCTTGCTTCAGATCCACAA
AATAAGAATAATTTTGCTTCATGGAGACAAGGTGAGCTTGAGAGAATGAGTCCTAATGATCTGCTGGCAATCTCGAGGTCTGAC
TATTATTGTTGGTGCTTGGATAATAAAGATTATATTTAA
SEQIDNO: 392
ACCAAATACTCTTTTCAAGACCATTAATTCCCGTTACAACAAATCATCTAATTTCTAGAATTAGCAAGTTTATTGACGCACGGAAG
CAAGAATAAATGAATAATACTTCCAAAAAGGGGACAGTTTTATTCAAATCCAGGATGCCCAAGGCTTAAAGGTGCCCTATATATA
AGGTGGGGCTAAAGTTGGCACAAATTTGGAAAATTTAAATTTAATCCTATTAACTTCATACAAATAACCAGCTAATTAAATAAACA
AGAGAAGAAAAATAAAAGAAATTCCAAGAAATTGTATATTTTGTCATCAACATAGAATCAACCAAATTATCGAAATGAGTTGCAGT
TTGCGAAAACTACAAATTGGAGACCAAAATGTCAGCCGATGTGAGTGTGGATCTTCTATTCAAGGGGTCAGTCAAAAAGTTCAC
ATGTGCTTCTTTCCTAGCGGAAACCCGGGCACGCCTACATACGTCCAGGGACAATTTTATGTAGTGTGAAGGTGGGTCCTAGAA
ATTTTCAAATTTCTTATAATTTATAAAGAAATTTTTTGAGGAACTAACTTAAAAATAACTTATGACCTACCTTATCTTCAAAAATCAT
AGTATTTGTATAGAAAATTATATTTAGAAAGTTTTGGTAACCATTTGGTTTTCATTTCTAGATTCTCCCCTGCATACGTCAAAAAGA
ACGTTTAACCATATCATGCATGGCGGTACTTGAAGACCTGGAGCTCTTTACAGTGAGTCGCATGTTCCTCAAAAGTGTCCAAAC
ATGTCTTGCGACAAACTGGACATGCATTACCAACCGGAATAATAGAATCATGAGGGGATACTTGATGATAATACGATATTTCATT
GGTGATATATGTTGACCTAAACCCTTTATAGGGATAGCCAGTAGTTAAATTAAATATAGAAAGCAACATATGAGTGTTTATAAAAA
TCAAGATTTTATGTTTTTGCATGGGTATCTCACAGAGGGCCGGGAAGGAACCAATTGACAATAAATAAAAGGAATAAAGTAATAG
AAATAGTAATGAGAGAGGACAATTATTCTCTACGATGTGGAGTAGGACCATGCATGGGTTTATCAAGCATGTTCATTTGTTCTTG
GAATATGCAGTCAACCATTCAAGAGACTAGAGATAGAGACTTCCCCACGTGAGCACTAACGCAAAAAGTACTTAATTAAGGAAT
CATTCAAGAGAATTCTCAGGTTGTACTATCAACTACTTTTAACTTTCTAAGTTCTAACCCCGACTATGTTTTACCAGCATGTACTTC
AGGCTTACTACGTACTCACCATCACTGCATGAAAAGTTAATAGGATCGATCCATTTTATATATGAAAATAGAAATATATATATATA
CCTTTAATTTCCAGGTCTACTGATGTTATAAATACATTCAACATTCTTCCCTTTCCTCCTCAAACCCTCTCAACATTTCTTCTCCTA
CAACTACAAGCTAAGAAACAACTACTACTATTATTTTATTCCAAAATGGATCCTTCAGAAACTCTAAGGTAACAACCATAAATCAA
TATTTCTGCATGCTTATATATATATATATATATATATATATATATATATATATATATATATATATATATATATATATATATATATATATA
TATATATATATATATATATATATATATATATATAATCAACAACATTTATATTCAAATATATAACATCCTAAACTTAATTTACCATGCAT
TAATATTTTGTCCAGGAACAAGTGTGCAGCCTGCTATCGGCAATTCAACAGATTTGAGCACCTAGTGGAACACATGAGAACTTCA
TATCACTCGGTTCATGAACCCATGTGTGGAATTTGTGGAAAACGATGCCGATCGTTTGAATCTCTTCGTGAGCATCTTATAGGTA
TATATATATTAATTACATGATGTATATTCAATTACTCATTAGAGTAATTGATCAATCAGTTAACGTTCTTAATTGATATAACTACAAG
ATATTAAATTCTTAATTAATTCACAGGGCCATTGCCAAAGGCAGAATGTGAAAGGGTGTTCCGCGACCGTGGATGTGACATTTG
CTTAACCATCCTTACCAGCCGTTATGCTCTCAGGGCTCACCGTGATGCATGCCAACTCTCTTGTGGCAATAACGTAAATCCTCG
ATCTTTTAAAAAAACTATCTATTTTCTCGATATATTGCGAGTGTACATGTGTTTTAATTAAGAATTATTTTGGCTATGATTACTTAAA
TTCAGGGTTTATTACACCGATTTGCTAACATGGGGATACAAGATGATTTAAGAATCGATAGCGGCAAATCAAGAGCAGTTGCACT
TGCGTGCAAAATGGTCGGTGGTGGTAGTGATGGTTCATTGGATCTTTGTGCAAAAATTTGCATCATTGATGAATATGAAAACACA
ATATTTCGTTCATATGTCAAACCGCAGCTTCCTGTTACAAATTACAGGTAAAGATCTTCTTTCACCTTCTTAATTTCCCCATTTGTT
TAATCCCCACATATATAATCTTTTATAATTAATAAAAATGTTGTGACATTTAATTCATAGGTATGAGACAACAGGCATTCGGCCAG
AATACCTTCGGGACGCAATGCCACTTAGGCAGGTTCAAAGAAAGATTCAAGATTTTCTATGCAATGGTGAACCCATTTGGAAGAT
CCGCCCAAGAGGTGGGAAGGCAAGGATTCTTGTAGGTCATGGTTTGGATCATTATCTTAGATGCTTGGAATTCGATTACCCTTC
ACTTAAAATTAGGCAAGCAATCACACCTTCTAAATTTCAACATACTTTCTCAAGTTATCGACATATCCAATTTGACGTTAATTTGTA
TATATGCGCTAATTTTGTCACAGGGATACAGCAACCTATCCGCCATTAATGAAGACTAGCAAGCTCAGCAATTCATTGAAGTATC
TCACAAAAGCATACCTTGGGTAATCTTTGATACCTTAAACATTTAATTAAGGATTCGAAGTAAAATTAATTTTAATTTCTGTATTTT
GTTATTTACGTAGATATGATATTCAAGTTGGGATACAAGATCCTTATGAAGACTGTGTTGCAACAATGAGACTTTACAGGAGGAT
GAGATCACAGGCTCATAGAAACGAGAACCATCCGCTTGCTTCAGATCCACAAAATAAGAATAATTTTGCTTCATGGAGACAAGG
TGAGCTTGAGAGAATGAGTCCTAATGATCTGCTGGCAATCTCGAGGTCTGACTATTATTGTTGGTGCTTGGATAATAAAGATTAT
ATTTAAACAAAGGGAAGGATTGATCGAATCAAGATTTAATAGCTAAATTGAACAAATAATTAAGTTTACAAAAACTATTAGTTATGA
GTATGAATAGGAATAAGGCATGGAAATTTGTCCATGATTTGTAGTAGATGAGATAATGCATACTACGTACGATTTTATGAGACGC
GTGTCTGTACGAATTAATATTTCTGTTAGAATATGAATAAATATGTGTTGTAATAATCTTTGTAAAGAAACGTCTGTTATTTGAGAT
TGAGAATTAGTACACACTTGAAACGCATACATAGTAATGCAAGAAATTCCATGAAGACATCAATTAATAGTAACGTACACAACTAT
CATGCCCTTTAATATTTCCATAAAGACCATGAAAAACAATTAGAACTGAAATGAGAAGGAACCACCTGAAGTGAATAATCGAAGA
ATCACAATGGGAAAATGGACTTTCGTTAGAATTCATGACTTACGATCAATGTGACGTGGGATATGGTGGAAAGATCGAGTCTCA
GAACATTTATCTCACGTACAAACTTAAAGCTATGTTCTATTAATTCAAGAGGTAATTAGTTTAAAATGTTAAAAACATAAAATTGCT
TTTGAGACCTATTATAATTACTTCTGTGATCGGTTCTAAAATCCATAAAAAAAACATTGTGATTCATTCTGGAATTGGTTTTAACTT
TTAACATTACAACATCCGTTGGTTTTGGTTTCAAGAGTCGTTGAACACTCATTATTAACAAAAAAATATGGCAAATGATAAATAATT
GTCTTAAACCACATTGATGCAATTAAGAGATATAGAGTGGTCTCATACTGTTGGGAAAAATAAACTGTGTATTATTCTCTTTAATC
ATTTATGTACTTTTGTCTTAGATAAGGACCGTTTTTTAATTTTATTATTCTGGACAATTTTTTAAATTTTGTCTTACAGAGGACTAAT
TTTGTATTCTCTTTTACACATCACCGTTTCTATAAAATTGTTTTAAATTTTTTTTTTCCGGCAACAACAAGCTAAAACTCCATTAAAA
CCCAGTAAAAGACACTGGGAGAACAAAGAGATTACAGGTTATAAAAAGGATTTGACATCCACAAGTTCAAATCCGGTAATTTTAA
GTTACTCCTATTGAACAACCAATAATAAGCAAACTCCTGAGATCTCCTTAAGCAAAACTCTTTCTCCACTCTTTTATTGTTAAAAAT
AATATCATTTCTATGAATCCATGTAGATTTGAGCAAAGCCACCACCAGCACCTTGACCATTTTGATTCTCGACGGGGCTCGGAAA
ACACAATCAATCCAAGCAAAGCAATCCCTTAGAGTTTCAAAAGAAGGAATAGAGCAGTTGACCAACAAGCCGAGTCTCGCCCAT
AACCAGGTTACGAAATCACATTAGAAGAACACGTGACTAGTATCCTCCGCTTGATCACCACAAATCTCACAATTTGAGCTTGGGA
CATCGAAGCCCATCTCCACTAGATTTTTTTCGAACCGCAATTC
SEQIDNO: 393
MDPSETLRNKCAACYRQFNRFEHLVEHMRTSYHSVHEPMCGNCGKRCRSFESLREHLIGPLPKAECERVFRDRGCDICLTILTSRYA
LRAHRDACQLSCGNNGLLHRFANMGIQDDLRSDSGKSRAVALACKMVGGGSDGSLDLCAKICIIDEYENTIFRSYVKPQLPVTNYRY
ETTGIRPEYLRDAMPLRQVQRKIQDFLCNGEPIWKIRPRGGKARILVGHGLDHYLRCLEFDYPSLKMRDTATYPPLMKTSKLSNSLKY
LTKAYLGYDIQVGIQDPYEDCVATMRLYRRMRSQAHRNENYPLASDPQNKNNFASWRQGELERMSPDDLLAISRSDYYCWCLDNK
DYI
SEQIDNO: 394
ATGGATCCTTCAGAAACTCTAAGGAACAAGTGTGCAGCCTGCTATCGGCAGTTCAACAGATTTGAGCACCTAGTGGAACACATG
AGGACTTCATATCACTCGGTTCATGAACCCATGTGTGGAAATTGTGGAAAACGATGCCGATCCTTTGAATCTCTTCGTGAGCATC
TTATAGGGCCATTGCCAAAGGCAGAATGTGAAAGGGTGTTCCGTGACCGTGGATGTGACATTTGCTTAACCATCCTTACCAGCC
GCTATGCTCTCAGGGCTCACCGTGATGCATGCCAACTCTCTTGTGGCAATAACGGTTTATTACACCGATTTGCTAACATGGGGA
TACAAGATGATTTACGAAGCGATAGCGGGAAATCAAGAGCAGTTGCACTTGCGTGCAAAATGGTCGGTGGTGGTAGTGATGGT
TCATTGGATCTTTGTGCAAAAATTTGCATCATTGATGAATATGAAAACACAATATTTCGTTCCTATGTCAAACCGCAGCTTCCTGT
TACAAATTACAGGTATGAGACCACAGGCATTCGGCCAGAATACCTCCGGGACGCAATGCCACTTAGGCAGGTTCAAAGAAAGA
TTCAAGATTTCTTATGCAATGGTGAACCCATTTGGAAGATCCGCCCAAGAGGTGGGAAGGCAAGGATTCTTGTAGGTCATGGTT
TGGATCATTATCTTAGATGCTTGGAATTCGATTACCCTTCACTTAAAATGAGGGATACAGCAACCTATCCGCCATTAATGAAGAC
TAGCAAGCTCAGCAATTCATTAAAGTATCTCACAAAAGCATACCTTGGATATGATATTCAAGTTGGGATACAAGATCCTTATGAA
GACTGTGTCGCAACAATGAGACTTTACAGGAGGATGCGATCACAGGCTCATAGAAACGAGAACTATCCACTTGCTTCAGATCCA
CAAAATAAGAACAATTTTGCTTCATGGAGACAAGGTGAGCTTGAGAGGATGAGTCCTGATGATCTGCTGGCAATTTCGAGGTCT
GATTACTACTGTTGGTGCTTGGATAATAAAGATTATATTTAA
SEQIDNO: 395
AACCCCGGCACGCCTACATACGTCAAAAAGAACATCCCTAACCATATCATGGCGGTACTTGAAGACCAGGTACTCTTTACAGTG
AACCGCATGTTCCCCAAAAGTGTCCAAACATGTCTTGCGACAAACTGGACATGCGTCACCAACCAGAAATAATGGAATCATGAG
GCGATACTTAATGATAGTACGATATTCTACTGGTGATATATGTTGACCTAAACCCTTTACAATAAATTAAATATATAGAAAGCAGC
ATATGAGTGTTTATAAAAATCAAGACTTTATGTTTTTGCATGGGTGTATCTCACAGAGGGACGGGAAGGAACCAAATTGACAGTA
AATAAAAGGAATAAAGTAATAGAAATAGTAATGCGAGAGGACAAATATTCTCTACGATGTGGAGTAGGACCATGCATGGGTTTAT
CAAGCATGTTCATTTGTTCTTTGAATATGCAGTCAACCATTCAAGAGACTAGAGATAGAGACTTCCCCACGTGAACACTAACGCA
AAAAGTTCTTAAGGAATCATTCAAGAGAATTCTCAGGTTGTACTATCAACCACCTCTAAGTTCTAACCCCGACTATGTTTTACCAG
CATGTACTTCAGGCTTACTACGTACTCACCATCACTGCGTGAAAAGTTACTTGGATCCATTTTATATATACCTTTAATTTCCAGGT
CTACTGATGTTATAAATACCATCAACATTCTTCCATTTCCTCCTCAAACTCTCTCAACATTTCTTCTCCCACAACTACAAGAAACCA
CTATCATTTTATTTCAAAATGGATCCTTCAGAAACTCTAAGGTAACGACCATAAATCAATATTTCTGCATGCTTATTATATATATCA
AAGATGACATGCCACTTGCATTCCATGATAATATATATAATTATTAATTAACAGCATCACTTGTGATCAGTCTGCATTTATATTCAA
ATATATAACATCCCGTAACTTATATCTTACCATGCATTGATATTTTGTCCAGGAACAAGTGTGCAGCCTGCTATCGGCAGTTCAA
CAGATTTGAGCACCTAGTGGAACACATGAGGACTTCATATCACTCGGTTCATGAACCCATGTGTGGAAATTGTGGAAAACGATG
CCGATCCTTTGAATCTCTTCGTGAGCATCTTATAGGTATATATATTAATTACATAATGTATATATCAATTACTCATTTGAGTAAATC
AATCAACTAACGCTTTGATATAATTAATTACATGATGTTAATTTCTTAATTAATTCACAGGGCCATTGCCAAAGGCAGAATGTGAA
AGGGTGTTCCGTGACCGTGGATGTGACATTTGCTTAACCATCCTTACCAGCCGCTATGCTCTCAGGGCTCACCGTGATGCATGC
CAACTCTCTTGTGGCAATAACGTAAATCCTCGATCTTTAATTAAACAAAACTATCTATTTTCTCGATATGTGTAAGTGTACGTACAT
GTGTTTTAATCAAGAATTATTTTGGATATGATTACTTCAATTCAGGGTTTATTACACCGATTTGCTAACATGGGGATACAAGATGA
TTTACGAAGCGATAGCGGGAAATCAAGAGCAGTTGCACTTGCGTGCAAAATGGTCGGTGGTGGTAGTGATGGTTCATTGGATC
TTTGTGCAAAAATTTGCATCATTGATGAATATGAAAACACAATATTTCGTTCCTATGTCAAACCGCAGCTTCCTGTTACAAATTAC
AGGTACAGATCTTCTTTCACCTTCTTAATTGCCCCATTTGTTTAATCGATATCCCCATATATATAATCTTTTATAATTAATACAATAG
TTGTGACATTTAATTAATTCATAGGTATGAGACCACAGGCATTCGGCCAGAATACCTCCGGGACGCAATGCCACTTAGGCAGGT
TCAAAGAAAGATTCAAGATTTCTTATGCAATGGTGAACCCATTTGGAAGATCCGCCCAAGAGGTGGGAAGGCAAGGATTCTTGT
AGGTCATGGTTTGGATCATTATCTTAGATGCTTGGAATTCGATTACCCTTCACTTAAAATGAGGCAAGCAATCACACTTTCTAAAT
TATAACACACTTTCTCAAGTTATCGACATATCCAATTTGACGTTAATTTGTATATCTGCGCTAATTTTATCACAGGGATACAGCAA
CCTATCCGCCATTAATGAAGACTAGCAAGCTCAGCAATTCATTAAAGTATCTCACAAAAGCATACCTTGGGTAATTAATCTTTTAT
ACCTTAAACATTAATTTAAGAATTCGAGGTAAAATTAATTTTAATTTGTGTATTTTCTTATTTACATAGATATGATATTCAAGTTGGG
ATACAAGATCCTTATGAAGACTGTGTCGCAACAATGAGACTTTACAGGAGGATGCGATCACAGGCTCATAGAAACGAGAACTAT
CCACTTGCTTCAGATCCACAAAATAAGAACAATTTTGCTTCATGGAGACAAGGTGAGCTTGAGAGGATGAGTCCTGATGATCTG
CTGGCAATTTCGAGGTCTGATTACTACTGTTGGTGCTTGGATAATAAAGATTATATTTAAACAAAGGGAAGGATTGGTCAAATCA
AGATTTGATAGCTAAAGTGAACAAATAATATTAAGTTTGCAAAAATTAATAGTTATGAGTATGAATAGGAATAAGGCATGGAAATT
TGTCCATGATTTGTAGTGGATGAGATAATGCATAGTACGATTTTATGAGACGCGTGTCTTTACGAATTAATATTTCAGTTAGAATA
TGAATAAATATGTGTTGTAATAATCTTTGTAAAGAAACGTCTGTTATTTGAGATTGAGAATTAGTACGCGTACATAGTGATGCAAG
AAATTCCTTGAAGGCATCAATTAATAGTAACACAACTATCATGGCCTTTAATATTTCCATAAGGACCATGAAAAACAATTAGAACT
GAAATGAGAAGGAACCACCTGAAGTGAATAATCGAAAAATCACAATGGGAAAATGGAGTTTCGTTAGAATTCATGACTTACGATC
AATGTGACGTGGGATATGGTGGAAAGATCGAGTCTCAGAACATTTATCTCACGCTACAAACTTAAAGCTATGTTCTATTAATTCA
AGAGGGACTAATTAGTTTAAAATGTTAAAAACATAGAATTGTTATATATTGAGACCTATTATAATTACTTCTGTGATCGGTTCTAAA
ATCCATAATAAAAACATTGTGATTCATTCTGGAATTGGTTTTAACTTTAAACATTACAACATCCGTTGGTTTTGGTTTCAAGAGTCG
TTGAACACTCATTATTAACAAAAAAAATATGGCAAATGATAAATAATTGTCTTAAACCACATTGATGCAATTAAGAGATATAGAGT
GGTCTCATATTGTTGGGAAAAATAAACTGTGTATTATTCTCTTTGATCATTATGTACTTTTGTCTTAGATAAGGACCGTTTTTAAAT
TTTATTATTCTGGACAGTTTTTAAAAGTTTGTCTTATAGAGGACTAATATTGTATTCTCTTTTACACATCACCGTTTCTATAAAAATA
AGGACCAATTTTGTAATTTCTCACATACGAACCAATTTTCTCATTTTGTCTACATGAAATTATAGAGACCAAAAATTCAATTTACTG
AGACAAATATATTCTAACATTGATTTGACCTTAAGAACAATTACTAACAATGTATATAATATAATGTTGACTTAGCCATTTCCTAAA
TTTGTTATTCAATAGATCCTCTAGTTATTCAGTTATTCTTTTATGTATTATTCGGATTAAACAGATAACCATATTACATGGCATTGTT
GGTTTGGCATCACTTGTCGCTTCAAAAGCCACTTAGTTTTTTTTTTTAAACTAAAATTCGTTCTTACTCATTCGAAAGAAACGATAT
TATTTGTTATTGATCTGTAAACAAAACCAAGTTTTGTAATGAACCTTTCGTTTCTGACCACTAGCGTCCATCTCTCAACCACCAAA
GAATAAGAATCTTGAAAGTAAAAATAAAAACCTGGAAACTAAAAATAAAAAGGTAGAAACTAAGAATAAAAATCTAATATCATATT
CTTTT
SEQIDNO: 396
MGPSETLRNKCAACYRQFNRFEHLVEHMRTSYHSVHEPMCGICGKRCRSFESLREHLIGPLPKAECERVFRDRGCDICLTILTSRYA
LRAHRDACQLSCGNNGLLHRFANMGIQDDLRIDSGKSRAVALACKMVGGGSDGSLDLCAKICIIDEYENTIFRSYVKPQLPVTNYRYE
TTGIRPEYLRDAMPLRQVQRKIQDFLCNGEPIWKIHPRGGKARILVGHGLDHYLRCLEFDYPSLKIRDTATYPPLMKTSKLSNSLKYLT
KAYLGYDIQVGIQDPYEDCVATMRLYRRMRSQAHRNENHPLASDPQNKNNFASWRQGELERMSPNDLLAISRSDYYCWCLDNKDYI
SEQIDNO: 397
ATGGGTCCTTCAGAAACTCTAAGGAACAAGTGTGCAGCCTGCTATCGTCAATTCAACAGATTTGAGCACCTAGTGGAACACATG
AGAACTTCATATCACTCGGTTCATGAACCCATGTGTGGAATTTGTGGAAAACGATGCCGATCGTTTGAATCTCTTCGTGAGCATC
TTATAGGGCCATTGCCAAAGGCAGAATGTGAAAGGGTGTTCCGCGACCGTGGATGTGACATTTGCTTAACCATCCTTACCAGCC
GTTATGCTCTCAGGGCTCACCGTGATGCATGCCAACTCTCTTGTGGCAATAACGGTTTATTACACCGATTTGCTAACATGGGGA
TACAAGATGATTTAAGAATCGATAGCGGCAAATCAAGAGCAGTTGCACTTGCGTGCAAAATGGTCGGTGGTGGTAGTGATGGTT
CATTGGATCTTTGTGCAAAAATTTGCATCATTGATGAATATGAAAACACAATATTTCGTTCATATGTCAAACCGCAGCTTCCTGTT
ACAAATTACAGGTATGAGACAACAGGCATTCGGCCAGAATACCTTCGGGACGCAATGCCACTTAGGCAGGTTCAAAGAAAGATT
CAAGATTTTCTATGCAATGGTGAACCCATTTGGAAGATCCACCCAAGAGGTGGGAAGGCAAGGATTCTTGTAGGTCATGGTTTG
GATCATTATCTTAGATGCTTGGAATTCGATTACCCTTCACTTAAAATCAGGGATACAGCAACCTATCCGCCATTAATGAAGACTA
GCAAGCTCAGCAATTCATTGAAGTATCTCACAAAAGCATACCTTGGATATGATATTCAAGTTGGGATACAAGATCCTTATGAAGA
CTGTGTTGCAACAATGAGACTTTACAGGAGGATGAGATCACAGGCTCATAGAAACGAGAACCATCCGCTTGCTTCAGATCCACA
AAATAAGAATAATTTTGCTTCATGGAGACAAGGTGAGCTTGAGAGAATGAGTCCTAATGATCTGCTGGCAATCTCGAGGTCTGA
CTATTATTGTTGGTGCTTGGATAATAAAGATTATATTTAA
SEQIDNO: 398
TCCCGTTACAACAAATCATCTAATTTCTAGAATTAGCAAGTTTATTGACGCACGGAAGCAAGAATAAATGAATAATAATTCCAAAA
AGGGGACAGTTTTATTCAAATCCAGGATGCCCAAGGCTTAAAGGTGCCCTATATATAAGGTGGGGCTAAAGTTGGCACAAATTT
GGAAAATTTAAATTTAATCCTATTAACTTCATACAAATAACCAGCTAATTAAATAAACAAGAGAAGAAAAATAAAAAAAATTCCAAG
AAATTGTATATTTTGTCATCAACATAGAATCAACCAAATTATCGAAATGAGTTGCAGTTTGCGAAAACTACAAATTGGAGACCAAA
ACGTCAGCCGATGTGAGTGTGGATCTTCTATTCAAGGGGTCAGTCAAAAAGTTCACATGTGCTTCTTTCCTAGCGGAAACCCGG
GCACGCCTACATACGTCCAGGGACAATTTTATTTAGTGTGAAGGTGGGTCCTAGAAATTTTCAAATTTCTTATAATTTATAAAGAA
ATTTTTTTGAGGAACTAACTTAAAAATAACTTAGGACCTACCTTATCTTTAAAAATCATAGTATTTGTATAGAAAATTATATTTAGAA
AATTTTGGTAACCATGTGGTTTTCATTTCTAGATTCTCCCCTGCATACGTCAAAAAGAACGTCTCTAACCATATCATGCATGGCG
GTACTTGAAGACCAGGAGCTCTTTACAGTGAGTCGCATGTTCCTCAAAAGTGTCCAAACATGTCTTGCGACAAACTGGACATGC
ATTACCAACCGGAATAATAGAATCATGAGGAGATACTTGATGATAATACGATATTTCATTGGTGATATATGCTGACCTAAACCCTT
TATAGGGATAGCCAGTAGTTAAATTAAATATAGAAAGCAACATATGAGTGTTTATAAAAATCAAGATTTTATGTTTTTGCATGGGT
ATCTCACAGAGGGCCGGGAAGGAACCAATTGACAATAAATAAAAGGAATAAAGTAATAGAAATAGTAATGAGAGAGGACAATTA
TTCTCTACGATGTGGAGTAGGACCATGCATGGGTTTATCAAGCATGTTCATTTGTTCTTGGAATATGCAGTCAACCATTCAAGAG
ACTAGAGATAGAGACTTCCCCACGTGAGCACTAACGCAAAAAGTACTTAAGGAATCATTCAAGAGAATTCTCAGGTTGTACTATC
AACTACTTTTAACTTTCTAAGTTCTAACCCCGACTATGTTTTACCAGCATGTACTTCAGGCTTACTACGTACTCACCATCACTGCA
TGAAAAGTTAATAGGATCGATCCATTTTATATATGAAAATAGAAATATATATATACCTTTAATTTCCAGGTCTACTGATGTTATAAA
TACCTTCAACATTCTTCCCTTTCCTCCTCAAACCCTCTCAACATTTTCCCTTTCCTCCTCAAACCCTCTCAACATTTCTTCTCCTAC
AACTACAAGCTAAGAAACAACTACTACTATTATTTTATTCCAAAATGGGTCCTTCAGAAACTCTAAGGTAACAGCCATAAATCAAT
ATTTCTGCATGCTTTTATATATATATATATATATATATATATATATATATATATATATATATATATATATATATATATATATATATATAT
ATATATATATATATATAATCAACAACATTTAAATTCAAATATATAACATCCTAAACTTATATCTTACCATGCATTAATATTTTGTCCA
GGAACAAGTGTGCAGCCTGCTATCGTCAATTCAACAGATTTGAGCACCTAGTGGAACACATGAGAACTTCATATCACTCGGTTC
ATGAACCCATGTGTGGAATTTGTGGAAAACGATGCCGATCGTTTGAATCTCTTCGTGAGCATCTTATAGGTATATATATATTAATT
ACATAATGTATATTCAATTACTCATTAGAGTAATTGATCAATCAATTAACGTTCTTAATTGATATAACTACAAGATACTAAGTTCTTA
ATTAATTCACAGGGCCATTGCCAAAGGCAGAATGTGAAAGGGTGTTCCGCGACCGTGGATGTGACATTTGCTTAACCATCCTTA
CCAGCCGTTATGCTCTCAGGGCTCACCGTGATGCATGCCAACTCTCTTGTGGCAATAACGTAAATCCTCGATCTTTTAAAAAAAC
TATCTATTTTCTCGATATATTGCGAGTGTACATGTGTTTTAATTAAGAATTATTTTGGCTATGATTACTTAAATTCAGGGTTTATTAC
ACCGATTTGCTAACATGGGGATACAAGATGATTTAAGAATCGATAGCGGCAAATCAAGAGCAGTTGCACTTGCGTGCAAAATGG
TCGGTGGTGGTAGTGATGGTTCATTGGATCTTTGTGCAAAAATTTGCATCATTGATGAATATGAAAACACAATATTTCGTTCATAT
GTCAAACCGCAGCTTCCTGTTACAAATTACAGGTAAAGATCTTCTTTCACCTTCTTAATTTCCCCATTTGTTTAATCCCCACATATA
TAATCTTTTATAATTAATAAAAATGTTGTGACATTTAATTCATAGGTATGAGACAACAGGCATTCGGCCAGAATACCTTCGGGACG
CAATGCCACTTAGGCAGGTTCAAAGAAAGATTCAAGATTTTCTATGCAATGGTGAACCCATTTGGAAGATCCACCCAAGAGGTG
GGAAGGCAAGGATTCTTGTAGGTCATGGTTTGGATCATTATCTTAGATGCTTGGAATTCGATTACCCTTCACTTAAAATCAGGCA
AGCAATCACACCTTCTAAATTTCAACATACTTTCTCAAGTTATCGACATATCCAATTTGACGTTAATTTGTATATCTGCGCTAATTT
TATCACAGGGATACAGCAACCTATCCGCCATTAATGAAGACTAGCAAGCTCAGCAATTCATTGAAGTATCTCACAAAAGCATACC
TTGGGTAATCTTTGATACCTTAAACATTTAATTAAGGATTCGAAGTAAAATTAATTTTAATTTCTGTATTTTGTTATTTACGTAGATA
TGATATTCAAGTTGGGATACAAGATCCTTATGAAGACTGTGTTGCAACAATGAGACTTTACAGGAGGATGAGATCACAGGCTCAT
AGAAACGAGAACCATCCGCTTGCTTCAGATCCACAAAATAAGAATAATTTTGCTTCATGGAGACAAGGTGAGCTTGAGAGAATG
AGTCCTAATGATCTGCTGGCAATCTCGAGGTCTGACTATTATTGTTGGTGCTTGGATAATAAAGATTATATTTAAACAAAGGGAA
GGATTGATCGAATCAAGATTTAATAGCTAAATTGAACAAATAATTAAGTTTACAAAAACTATTAGTTATGAGTATGAATAGGAATAA
GGCATGGAAATTTGTCCATGATTTGTAGTAGATGAGATAATGCATACTACGTACGATTTTATGAGACGCGTGTCTGTACGAATTA
ATATTTCTGTTAGAATATGAATAAATATGTGTTGTAATAATCTTTGTAAAGAAACGTCTGTTATTTGAGATTGAGAATTAGTACACA
CTTGAAACGCATACATAGTAATGCAAGAAATTCCATGAAGGCATCAATTAATAGTAACGTACACAACTATCATGCCCTTTAATATT
TCCATAAAGACCATGAAAAACAATTAGAACTGAAATGAGAAGGAACCACCTGAAGTGAATAATCGAAGAATCACAATGGGAAAAT
GGACTTTCGTTAGAATTCATGACTTACGATCAATGTGACGTGGGATATGGTGGAAAGATCGAGTCTCAGAACATTTATCTCACGT
ACAAACTTAAAGCTATGTTCTATTAATTCAAGAGGTAATTAGTTTAAAATGTTAAAAACATAAAATTGCTATTGAGACCTATTATAA
TTACTTCTGTGATCGGTTCTAAAATCCATAAAAAAACATTGTGATTCATTCTGGAATTGGTTTTAACTTTTAACATTACAACATCCG
TTGGTTTTGGTTTCAAGAGTCGTTGAACACTCATTATTAACAAAAAATATGGCAAATGATAAATAATTGTCTTAAACCACATTGAT
GCAATTAAGAGATATAGAGTGGTCTCATACTGTTGGGAAAAATAAACTGTGTATTATTCTCTTTAATCATTTATGTACTTTTGTCTT
AGATAAGGACCGTTTTTTAATTTTATTATTCTGGACAATTTTTTAAATTTTGTCTTACAGAGGACTAATTTTGTATTCTCTTTTACAC
ATCACCGTTTCTATAAAATTGTTTTAAATTTTTTTTTCCGGCAACAACAAGCTAAAACTCCATTAAAACCCAGTAAAAGACACTAG
GAGAACAAAGAGATTACAGGTTATAAAAAGGATTTGACATCCACAAGTTCAAATCCGGTAATTTTAAGCTACTCCTATTGAACAA
CCAATAATAAACAAACTCCTGAGATCTCCTTAAGCAAAACTTTTTCTCCACTCTTTTATTGTTAAAAATTATATCATTTCTATGAATC
CATGTAGATTTGAGCAAAGCCACCACCAGCACCTTGACCATTTTGATTCTCGACGGGGCTCGGAAAACACAATCAATCCAAGCA
AAGCAATCCCTTAGAGTTTCAAAAGAAGGAATAGAGCAGTTGACCAACAAGCCGAGTCTCGCCCATAACCAGGTTACGAAATCA
CATTAGAAGAACAGGTGACTAGTATCCTCCGCTTGATCACCACAAATCTCACAATTTGAGCTTGGGACATCGAAGCCCATCTCC
ACTAGATTTTTTTCGAACCGCAATTCTGC
SEQIDNO: 399
MILAQLARALVSLWEVQCSNLLDSVMHRVLITHEGVRYEYYQCSGNPSREAPCVKFLPGNPMVRTALRMSRTRALSEIMGAWLPLIH
PPNKCAACYRQFNKKEHLVEHMRISYHSLHEPMCGICGKRCRSFESLRDHLIGPLPKAECERVFRDRGCTICLTILNSPYSLKVHQDR
CQLARGSNGVLHRFANMGIQDDLRIDSGKTRAVALACKMVGGGSDGSLDICARVCIIDEYENILFHSYVKPQLPVTNYRYETTGIRPEY
LRDAMPLRNVQRKIQDFLCNGEPIWKIRPRGGNARILVGHGLEHDMKCLEIQYSLVKIRDTAKYPPLMKTSKLSNSLKYLTKSYLGYDI
QNGIQDPYDDCVATMRLYRRMRSQAHRMEEYPLATDSQNKNNFSSWRQGELEKMSPDELLAISRDRVATKSNLEKMGIRAGKFGTI
VVIYYSYASYISTTDSDTDIQCFKLVHLRRGDCTETRPSCAPLNLMNRLHPGLMTLKAPKIVIIVIWKIDIESKKKKPKVMKRVGTVLELLL
LRRSLCAGFQDVLAPLFLPGFLPFGAGDVLESCCLGALFFAWGCL
SEQIDNO: 400
ATGATTTTGGCTCAGCTGGCAAGAGCGCTGGTCTCCCTCTGGGAGGTCCAGTGTTCGAACCTTCTGGACAGCGTGATGCATCG
GGTTTTAATAACCCATGAGGGTGTGAGGTACGAGTATTATCAATGCTCCGGTAATCCTTCGCGCGAAGCCCCGTGTGTGAAATT
TCTCCCTGGAAACCCTATGGTTCGAACTGCCCTTCGGATGTCTCGAACTCGGGCGCTCTCTGAAATAATGGGAGCGTGGCTTC
CGTTGATCCATCCGCCGAACAAATGTGCAGCATGCTATAGGCAGTTTAACAAGAAGGAGCACTTGGTGGAACACATGAGGATTT
CATATCACTCACTTCATGAACCCATGTGTGGAATTTGTGGCAAACGTTGTCGCTCTTTTGAATCTCTTCGTGATCATCTTATAGGA
CCATTGCCAAAGGCCGAATGTGAAAGAGTGTTTAGAGACCGTGGATGTACCATTTGCCTAACCATTCTTAATAGCCCATATTCTC
TAAAGGTTCACCAAGATCGATGCCAACTAGCACGTGGAAGTAATGGTGTACTGCATAGATTTGCAAACATGGGCATTCAAGATG
ACTTGAGAATCGATAGTGGGAAAACAAGAGCAGTTGCACTTGCTTGCAAGATGGTTGGAGGTGGTAGTGATGGATOTTTAGATA
TTTGTGCAAGAGTTTGCATCATTGATGAATATGAGAACATATTATTTCATTCCTATGTCAAGCCACAACTTCCTGTTACAAACTAT
AGGTATGAGACTACAGGCATCCGACCAGAGTACCTTAGGGATGCAATGCCACTCAGAAATGTCCAAAGAAAAATTCAAGATTTT
TTGTGTAATGGGGAACCGATATGGAAAATACGTCCAAGAGGTGGAAATGCGAGGATTCTTGTAGGACATGGTTTGGAGCATGA
CATGAAATGTTTGGAAATTCAGTATTCTTTAGTAAAGATAAGGGATACTGCAAAATATCCACCATTAATGAAGACAAGCAAGCTAA
GCAACTCACTCAAGTACCTAACAAAATCTTACCTAGGGTATGATATTCAAAATGGGATACAAGATCCTTATGATGATTGTGTTGC
AACGATGAGACTTTATAGAAGAATGAGATCACAAGCTCATAGAATGGAGGAGTATCCTCTTGCTACAGATTCCCAAAACAAGAAT
AACTTTTCATCATGGAGGCAGGGTGAGCTTGAGAAGATGAGCCCTGATGAATTGTTGGCAATCTCAAGGGACAGGGTCGCAAC
GAAGAGCAATTTGGAGAAGATGGGAATCAGAGCAGGGAAATTTGGTACAATAGTCGTAATATACTACAGCTACGCTAGTTACAT
CAGTACTACAGACTCTGATACGGACATCCAATGCTTCAAGCTTGTACACCTTCGACGAGGAGACTGCACAGAGACTAGACCATC
GTGCGCACCGTTAAACCTGATGAATCGCCTCCATCCCGGTTTGATGACCTTGAAAGCACCGAAAATTGTGATTATAGTGATATG
GAAAATCGACATAGAATCGAAGAAGAAGAAACCGAAAGTGATGAAGAGAGTCGGAACTGTTCTTGAACTCTTGCTGCTTAGGCG
TTCGTTATGTGCTGGGTTTCAGGACGTTCTTGCTCCCCTGTTCCTTCCCGGCTTTCTCCCCTTTGGTGCTGGTGATGTCTTGGA
GTCTTGCTGTTTGGGTGCCTTGTTTTTTGCTTGGGGCTGTTTGTGA
SEQIDNO: 401
GAAATGGCGGAGTTAAATAATCTGATCAGCATTGTGTCTCAAATCCGGACCTCGGTGGGTCAAGATCTCTGGATTTCCGAATTG
TCTTCAGATGGTCTTTTTGCTGTTCGTGATCTGCGCCATGCCATCGATGAGGTGATCACTACTCCGGCTCCTAACCCGACTATTT
GGATCCACATTATCCCGGTGAAAGTTATTTGTTTTGTTTGGCGAGTGTGTCTAGATCGTATCCCAACTGCTATCGCCCTATCAAA
GCGTGGTGTCCAAGTCCCGTCTAGTAGTTGCGCGTTTTGTATCAATGGAATTGATGAGGTAAATCACTTGATGGCCGACTGTCC
TTTTGCAAAAGAAATCCTCGAGTGGATTTTTGCCTGGTGTGGTATTCCTTTGCCTCAGTTCTCAAAAGTGGTGGACATTGTGGAC
TTCGCCGGTAACTGGGGTCGTTGTCCAAAGAAACGCAAAATTATGGTGGCTATTTGTTACGGATATATCTGGAGTTTATGGAAG
GCTCGGAATGACAAGGTGTTCAACAAATGCTCAACTCCGGTGGGGAAGATGAAAGACAATATCGTTTTATTGGTTTTCGATTGG
ATTAAAAACAGAGGAAATTATAAAAATTGTAATTTACCAGTGTGGAGTTGTTCCCCCTTTGATATTTTGTAATTTTGTCCCTTTCCT
GCCCCTACTTTTTTTCTCTTAAGGTGGTGTCTTAATAAAATTGCCTTTTCAAAAAAAAAGAACCAGATAAAAAAGAATGAAAAAAA
CTGGACCCAGTGTCAATGGTATCCATGCAAATTTTATGCTGCGTTCTTTTTCACAATCTTGTTTTTATTTGGGTACATTTTCAAAG
CTCGAATCTTCTTGGTCAAAAGTTTTTCATCCCAGAATCCCCACATCTTTTGTGCAACCCATGATAGTAACAACTACAAAAGGAAA
TCTAGGACCCGGTTCACCCATTACATATTATTACATGTATGTTTGTTGGCTCACTACTAATAAGGTCACTTCTTTACATAACTATAA
TGGAAAATTACAGATATTATCCCGCTATTATTAGTTTGCAAAATTACAGACTTGGTCTTCGCGTAATAGTAAAATTACATAAAATGT
CCGTGACTCCATGGGTCCTAACACTTTGCATGGAACATGGTCTTCGCTTGATAGTAAAATTACATAAAATGTCCATGACTCCGTA
GGTCCTTACACTTTGCATGAAATAGAGAAAATAGAGTGACTGTCCCTGGCGTAAAATGTAAAAGTTACAGAAATAGTCTATGCGT
AATAGTAAAATTATAGAGATGACTCCTATGCCATATATAGTGGAAAACAATATTATAGAAATAGTTTTTATGTCCTTACATATTTTA
GGAAATAAAGTTGTGTATTAGTTTATTTGGTTGAAAATAAACTTTTGTACATATAGGATTAACAAACTCTCGTGGCAACATGCCGC
TTAAAACCCTCGTGTTCCAATACATTTAAGTGATTAGCTAGGATTTTATGATTTTGGCTCAGCTGGCAAGAGCGCTGGTCTCCCT
CTGGGAGGTCCAGTGTTCGAACCTTCTGGACAGCGTGATGCATCGGGTTTTAATAACCCATGAGGGTGTGAGGTACGAGTATT
ATCAATGCTCCGGTAATCCTTCGCGCGAAGCCCCGTGTGTGAAATTTCTCCCTGGAAACCCTATGGTTCGAACTGCCCTTCGGA
TGTCTCGAACTCGGGCGCTCTCTGAAATAATGGGAGCGTGGCTTCCGTTGATCCATCCGCCGTTTCCAAAAAAAAAAAAAAACT
ATGTGAATTCAATAGTATAACATTTACTGCATGAAGATACAAAAAATGTCATCTTAATTTAGAAATATGCTAACTAATGTAATAGTG
TGTAACCAAGGTGAACACAATAGCATGTGAGCATGGAATATATTGCCATCATATAGAAGCATAAAGGAGCAAAAATACTCTCTAA
ATCCTAAGTGTTGACCAAATTCTGCAAGTATGAACTGTGTTTTTCTTGAATGGGGCATGTGCATTGAAGATTCCAAAGATGGAGA
CCGAAATCCGTATGCACTAGCGCGAAAAAAGGGTCTTAAAGGAATCAGTCCTTTTGTTGTACTAATCTTAATGTTATAAATACATT
GCACGTTCCTACCATTTCTTACTACAAGTCCTCTCAGTTTTTTGATAAAAAGATTGATTTAGTGATATCAAAGTTGAGAGAACAAT
TAATGGATTCTGAGACTCAAAGGTAGTAAGCATAAAATACTCGCATTTCATACAAAAACTAACACACAAGTIGTTAAAAATGATAC
AATAAAATCATCTTGCTTTTGATCTTATTATTAATTAGCATGTTTTTTACTAGGAACAAATGTGCAGCATGCTATAGGCAGTTTAAC
AAGAAGGAGCACTTGGTGGAACACATGAGGATTTCATATCACTCACTTCATGAACCCATGTGTGGAATTTGTGGCAAACGTTGT
CGCTCTTTTGAATCTCTTCGTGATCATCTTATAGGTATAATTACATTATCATTATTATATGTTTTAAAAAGTTTAGTTACTATATATT
GATGGTATTAACATATATGAATTCGGGTTTTCCACAGGACCATTGCCAAAGGCCGAATGTGAAAGAGTGTTTAGAGACCGTGGA
TGTACCATTTGCCTAACCATTCTTAATAGCCCATATTCTCTAAAGGTTCACCAAGATCGATGCCAACTAGCACGTGGAAGTAATG
TAATGCATCATTATATATATCCTTCAACTTATAATTTTCCAGCAAATGACTTATAGTCTAGTAGTATTAGGTGTTGACACAGTGATC
ACTCTCAGCCTTAATCCGTGAGGGTTCATTTCCTGTCATATGAAATAATGTTCTAGTTTTGGTTGTATACATAAATATAAAAACCA
AAGAGCTTCTACCTTAGCGGTATCCAGTGTTTTCCTCCCTCTTTGAGGTCGATGGTTTAAATCTTATATGAGACATAGGTGAATAT
ACGGAGTAGTTTAGGATTAGATTAGAATTGTCTTTTCAAAAAAAATATAAATACAAAAATATACCCAATTTTGATATATTTATATAT
GTTCCAGGGTGTACTGCATAGATTTGCAAACATGGGCATTCAAGATGACTTGAGAATCGATAGTGGGAAAACAAGAGCAGTTGC
ACTTGCTTGCAAGATGGTTGGAGGTGGTAGTGATGGATCTTTAGATATTTGTGCAAGAGTTTGCATCATTGATGAATATGAGAAC
ATATTATTTCATTCCTATGTCAAGCCACAACTTCCTGTTACAAACTATAGGTGCTATATAGTTTTCCATAATAAAATTATAAAACAA
AGTTGCTATTTTCTTATTTAAGAAAGTATGCAGGTATGAGACTACAGGCATCCGACCAGAGTACCTTAGGGATGCAATGCCACTC
AGAAATGTCCAAAGAAAAATTCAAGATTTTTTGTGTAATGGGGAACCGATATGGAAAATACGTCCAAGAGGTGGAAATGCGAGG
ATTCTTGTAGGACATGGTTTGGAGCATGACATGAAATGTTTGGAAATTCAGTATTCTTTAGTAAAGATAAGGCACGTATATATATT
TAGCTACTTTATTTAATATTTATACATATATATAATTAGTCCCATAACTTATTGTACTTAACTAATAGTAATTATATGCACTTGTTATT
GGATAGGGATACTGCAAAATATCCACCATTAATGAAGACAAGCAAGCTAAGCAACTCACTCAAGTACCTAACAAAATCTTACCTA
GGGTAAAGACATTACCTTTATTTTTATAAAGATATGTGATATGTTAGTATGTTCCTGATGAATTTTTATTGTATAGGTATGATATTC
AAAATGGGATACAAGATCCTTATGATGATTGTGTTGCAACGATGAGACTTTATAGAAGAATGAGATCACAAGCTCATAGAATGGA
GGAGTATCCTCTTGCTACAGATTCCCAAAACAAGAATAACTTTTCATCATGGAGGCAGGGTGAGCTTGAGAAGATGAGCCCTGA
TGAATTGTTGGCAATCTCAAGGTCTGATTACTACTGTTGGTGCTTAGATAGCAAAGATGTGGCTTGAAATGAAGGGAAGATTGAA
GAAAATAGCTAAACTGATTTAAAACTCATCAAATATAAATGGAATAAACATGGATACACATATATTTAAAATGTTTTACATCTCTAT
TAATTTAGAAGTAGTAAGAAGGCAGCTGTATTGCTATGTAAGCAGTGGCGGATCCAGAAAAAAATTTAAGTGGGTGCAATCTAAA
TTTTTATAAGATTTATATTAATTTTCTAGTATTTTAAAATATAAAATCATATATGTTATATAGAAAAATTGGAAAAATTAATATTTAAG
TGGGACACTCCACCCCCTTGGCTAAGGGTGGATCCGCCACTAAGTTGAGTGAAAATAAATACATCTTTGTTTGCTAAGAATGTT
GAGCACATAGTATCCATACTTGAATCCTATATTCACTAAAGGGTTAGTTATATATGTAAAACATTCAATTATATTATTCTTTTAGAT
AAAACCTTAACTTTAAACATTTGTTTAATAAAATCTTTTCATTTTCTTTCGAATAAGATCCTGGACTTCTTTTAATTTTTTTCTAAAAA
ATATTCATATTTATTTCTTTTTTGAAAAAAAATCTCTTTGTTTATTTCTTTCTAGAAAAACCTTCAACTTATTTTTACGAAATATAAGG
CTAAATTTTTTTCAAAAGAAAATGTCTAAAATTAGAAAATTAAAAATAAACTTGCTAATATAATTAATACAATATATATATATATATAT
TATATATATATATATATATATATATATATATATATATATCATCAGGGACAGGGTCGCAACGAAGAGCAATTTGGAGAAGATGGGAA
TCAGAGCAGGTTCGATCCTATGTGCTTTTTGTAGAAATGCCACTGAAATGGCGAATCATGTGTTTTGTAGCTGCCCATTAAAGAA
ATATGGAATAAAGTGTATGGATGGTGGGGTCAATCATTTGGGGACAAAAATAATGTGGAGGACATACTGGAAGATCTAGTGGCA
AACTCAAGCCACAAAAGAGATGAGAAAGTAAGATTAGTGGTGTGGGCCGCGGTGCTTTCTCAGATTTGGAAGTACCGAAATGAA
GTAATCTTTGAAAATAAAACCAAAACAGTGGAGGAGATTTTCTTAAAAGCCCAAGGTGAAGCAGCCTTGTGGTTGGAAAGTCGG
GCCTCAAAAGTGGAGATAGATAGATTGTCTTGAGCCTTGTCCCCAAACTAGCCTGTAAATTTATGTAACTCGTTGTTTGGCATCC
CAGTTTCTGGCTGGGATTCCTAGTTTGTTTTTAATACAGTTGCCTTTCAAATATATATATATATATATATATATATATATATATATAT
ATATATATATATTATATATATATATATATATATAGAAGTTGGTTTAAACGAGAACAATTATTGAATTTGAGAACGTGAGAACAAATTT
GGGTCACTTATTTTTTATTAATCATATGTGATTTTTCACATACGATTTACATGTATAAAATCATATGTGATTTTTATTAGTCATGTGT
AAATCATATGTGATTATATATGATGTTTTTTAGCATGAAAAATAAGTGGCTTAGATTTGTTCTTGTGTTCTCAAATTCAATAATTATT
CTCATTTGAACCGTCCTATATATATATATATATATATATAATATATATAATATATATATATATATATATATATTTTTATTATATATATAT
ATATATTGAAGCCAGGGAAATTTGGTACAATAGTCGTAATATACTACAGCTACGCTAGTTACATCAGTACTACAGACTCTGATAC
GGACATCCAATGCTTCAAGCTTGTACACCTTCGACGAGGAGACTGCACAGAGACTAGACCATCGTGCGCACCGTTAAACCTGA
TGAATCGCCTCCATCCCGTAAGTTGGCGTCTTCGGGCTAGTGACCACTAGGTAAACCAGCAGCCCCGGGTACACGAAACCTTC
GCTAGCACTGAGGCCTGCAAGAATGAGTAATATCTTTTCAAACTTGCAGCCGGGGAGGATCGAACCCGCGCCTCTCCTAAGAG
CGGCACTTGTCCTTACCATCCGGACCACGGAGGAGTGTGCATATATATATATATATATATATATATATATATATATATATATATATA
TATATATATATATATATATATATATATATATATATATATATATATATAGGGAGAGGTTATAATGAGAAATAAAAAATGGTGAGAACG
CGAGAACGATTCTGGACCAATCAATCTTAAGGATATATTAGTAATTTAATAATTCAATCATTTATTAAATCAAATTAATAAAACCCC
CATAAATTAGGAAATTGGATATTAAAAGATATATTTCGATCCTATAATCTCTCTAAAGATAAATTCAAAGATATTATATTTGACTTC
CATTCTTCCACCTGTTAATTACAGCGACCGAGTTTCTTCTTCCTTCTTCATCGTCGATCAAGAGTTTCTTCTTCCTTCTTCATCATC
GATAAAAAAAAATTAGGGTCAGTGACCGACTTTCTTCTTCCTTCTTCTTCATCATCGATCAAAAAAATTAGGGCCGACAAACCTTC
TTGTTCAATACAAAAGAATTAGGGCCGACTTCTTTCATCTTTCTTCTTGTTCCTCCTGATATCTTCATTAAACCGATAACTAATACA
TATATTTGTAATTGATTAAATACAAGATGGATTTTGCCGATTGAAGGGTTTGATGACCTTGAAAGCACCGAAAATTGTGATTATAG
TGATATGGAAAATCGACATAGAATCGAAGAAGAAGAAACCGAAAGTGATGAAGGTATGTATTAGTAATTTTTATATTTTCATAGTT
GAATGGATGATTGTTAGTATATGTTGTTAATATGCTGTTAACAGAGAGTCGGAACTGTTCTTGAACTGTAATTTGTATACGTCTAG
TTATAATCATTAATGATTATAACTTCCTAAATGTTTTAACGTTATAATCATTGATGATTTCATTCTTCACGTTATAAAACTATAGATA
TATATGTTTAGACTAGTTTAAATCTAGTATTATATTGGTCTATTGTACTTAGTAGTTTGAAAGTTGAATATGTTTATATGTAATGATT
AAAATTATTAAGATTAACGTAGTTTTGATTAAACAGGTAGTCAAAAGCAACGTAACTTCGACTTTAAAGGGATAGAATGTAAAGCA
TTGATTAACCAGAGAATGTGCAAAGGTTGTCGGGAATATGTCGGACATAATTGGGTTCGTCGGTTGTGAGCCTTACTGTTGGTT
CTGAGGGCCACTTTTGGTTTTGTCTTGGGTCCACGTTTATGTCCTGGTCCTGCTGCTTAGTTTTTGACGATTTTGTCCCTGAGGG
TGTATGGTGCTTTGTTTCGAATGTTTGGAGCTGTCCTAGAGCGTTGCTGACATTTTTGACTTTTTTGCCCCTAAGGGTGGAGGTT
CTTTCTTGTTGGTTCGGGTTGATGCTGCTGCAACTATGGTGGGCTTTGCGGATGGGGTTTTTGGACACTTTTACCCCTGTCTGC
TAGGTTGTCTTCTTGGTTGGTCCTTGTGCTGTTTTGAAGTTTTGATGTCTTGTTGCTCTGTTCAGCTTGCTGCTTAGGCGTTCGTT
ATGTGCTGGGTTTCAGGACGTTCTTGCTCCCCTGTTCCTTCCCGGCTTTCTCCCCTTTGGTGCTGGTGATGTCTTGGAGTCTTG
CTGTTTGGGTGCCTTGTTTTTTGCTTGGGGCTGTTTGTGATAGTAGTCGGTTAGGTAGTGTTGGTGTCTTAATGCTGGCATCGGT
TTATTTATTTAATTTGCAGCCGTTAAAAAACATAATTGAGGAACTTGTGAAGTTAGAATTGCAAGCAATGGGACTGATAAGAGCA
GTCAATAGATAATTGAAATTGTTTTTGTTTGAACTTTTATGGTATATGTGAATGAACATAATATATTTGTTTGAATTGAATTATTTAA
ATCATTTTTGTTTTGAAGTCAATTAGTAGACAACACTACATACAAATCAACAAAATATATGAATTATTGAAAGGTGAAACATATATG
AAAACTTAATTATGGATTAGGCTTAATCATTAATGATTAAAACTACAAAAACACAACAACAAATGTAAGCTAATTTGAATTCGCCAA
TGATTTTAACTTATAATCATACGTGATCAAGATTAGTATGTATTTATGTTCAATTGAAATCAACAATGATTTAATTTATAATCATGAA
CGACTTAACTTATAATCATCAATGATTAACTGATTAACTAAGTAATTATCAGAGTGTCTATACTTAGTAGTAGATATATTAAATCTG
TTTATACGTAATGATTTAACAAAATCACATAATACATACTTTTGTTTATTCTATAAAAACTTGTAAATAATATTGATTAAAATGTATAT
GAGTTTACAAACATTACATACAAATTACATTTATTCAGCCATAATCATATTGTGTTGGAAAATCATCAATAATTTAACCTCTAAGCA
TTGACTATAAAAATGTTTAAGAAATTATCAATAATTAACGTTAAAAAATATATAAAGACAACACAAATCATCAATGATTAACACATG
TTGTATTGTTGGGGTATTTTCCTTTGTATTGTCAACTTCAGCTACCTCCGGTTCAGTTTCAGGTTGAGCTTTAGATCTTGAGCTTG
ATTTTTGGATTGAGATTTGGGTCGACTTTTGGATTTCTCTAGTGTAGATTTATTTCCTGTTATTACAAATGTGAACTATGATAAGAA
TTACACATATTAAAAATCATAGATTATTGATTATGTGTATCTAGAGGAACCTGTATTTGTTAATCATTAATGAATAACCAACACATA
TTCATCAATAATTATAGTATGTTATAAATACAGGTCAGAAATGAAAGTTTATGTGTTTCACCTCTCATTACAATCATGATTAACCAA
CACATATTCATCAATGATTTTATTATGTTATAAATTCAAATCATAAATGATAGCTTATGTGTTCCAGTTTACATTTCAAACATCAATT
ATTATAAAACGAGACGAATTCCCGCGCGTTGTGCAAGGAGATTAAAATATGACATAAAAATAAACACTACGTAATTTAGAGTCAA
TTAAGTTAAATTGTTGTAAAATAAATTGGTTATAATTATTGGTAATGGTTTTATTTATTAAAATTAGAATCTTAATTTAAACAATACG
TTTAAATTAACATACATACACAATGTACATGCATATACACTTCATAATATAGTTACATCAAT
SEQIDNO: 402
MWLLNGFLTRNKCAACYKQFNKKEHLVEHMRISYHSVHEPMCGICGKRCRSFESLREHLIGPLPKAECERVFRDRGCTFCLNILSSP
HALRFHQDRCQLSRGNNGVLHRFANLRIHDGLRIENEKTRVVALACKMVGGGSDGSLDLCARICVIDEYENILFHSYVKPQLPVTNYR
YESTGIRPEYLRDAMPIRHVQRKIQDFLCNGEPIWKIRPRSGKARILVGHGLDHDMKCLELEDTSKYPPLMKTSKLSNSLKYLTKAYLG
YDIQNGIQDPYDDCVATMRLYRRMRYQAHRIEDYPLATDPQNKNNFTSWRQGELERMSPDELFAISRSDYYCWCLDSKDVA
SEQIDNO: 403
ATGTGGCTACTAAATGGTTTCTTGACTAGGAACAAGTGTGCAGCATGCTATAAGCAGTTTAACAAAAAGGAGCATCTGGTGGAA
CATATGAGGATTTCGTATCACTCGGTTCATGAACCTATGTGTGGAATTTGTGGGAAACGTTGTCGATCATTTGAATCTCTCCGTG
AACATCTTATAGGACCATTGCCAAAGGCCGAGTGCGAAAGGGTGTTTAGGGACCGTGGATGTACCTTTTGCTTAAACATCCTTA
GCAGCCCGCACGCTCTCAGGTTTCACCAAGATAGATGTCAACTATCGCGTGGAAATAATGGTGTCTTACATCGATTTGCTAACT
TACGCATTCATGATGGCCTGAGAATTGAAAATGAGAAAACAAGAGTAGTTGCTCTTGCCTGCAAAATGGTTGGGGGGGGTAGTG
ATGGTTCTTTAGATCTTTGTGCAAGGATTTGCGTCATTGATGAATATGAAAACATACTATTTCATTCTTATGTCAAGCCACAGCTT
CCTGTTACAAACTACAGGTATGAGAGCACAGGAATTCGACCAGAATACCTCAGGGATGCAATGCCAATAAGACATGTTCAAAGG
AAAATCCAAGATTTTTTATGCAATGGTGAACCGATATGGAAGATCCGGCCAAGAAGTGGGAAGGCAAGGATTCTTGTAGGACAT
GGTCTAGATCATGACATGAAATGCTTGGAACTAGAGGATACATCAAAATATCCACCCTTAATGAAGACTAGCAAGCTCAGCAACT
CTCTCAAGTACCTAACAAAAGCATATCTCGGCTACGATATTCAAAACGGGATACAAGATCCATACGATGATTGTGTTGCAACAAT
GAGACTATACAGAAGAATGAGATATCAAGCTCATAGAATAGAGGACTATCCTCTTGCAACAGACCCCCAAAACAAGAACAATTTT
ACTTCATGGAGACAGGGTGAGCTTGAGAGGATGAGTCCTGATGAACTTTTCGCAATCTCAAGGTCTGATTACTATTGCTGGTGC
TTAGATAGCAAAGACGTGGCTTAA
SEQIDNO: 404
ATGTGGCTACTAAATGGTTTCTTGACTAGGAACAAGTGTGCAGCATGCTATAAGCAGTTTAACAAAAAGGAGCATCTGGTGGAA
CATATGAGGATTTCGTATCACTCGGTTCATGAACCTATGTGTGGAATTTGTGGGAAACGTTGTCGATCATTTGAATCTCTCCGTG
AACATCTTATAGGTATAATACTTTTTCTTATCATTATTATTTATATTTTCTTATCACAACACCTTGATGGATATGTGTGTGTATACTA
ATTACTTCTTCAATTCTTTAGGACCATTGCCAAAGGCCGAGTGCGAAAGGGTGTTTAGGGACCGTGGATGTACCTTTTGCTTAAA
CATCCTTAGCAGCCCGCACGCTCTCAGGTTTCACCAAGATAGATGTCAACTATCGCGTGGAAATAATGTAATCAATTGTATCCTA
AAACTAATTAATATTTTTTTCTTTTTTAGATATATAAATGTACTGAACTATAAGCTTATACCCCTTTTTGTGATTTAATCAATATTCCA
GGGTGTCTTACATCGATTTGCTAACTTACGCATTCATGATGGCCTGAGAATTGAAAATGAGAAAACAAGAGTAGTTGCTCTTGCC
TGCAAAATGGTTGGGGGGGGTAGTGATGGTTCTTTAGATCTTTGTGCAAGGATTTGCGTCATTGATGAATATGAAAACATACTAT
TTCATTCTTATGTCAAGCCACAGCTTCCTGTTACAAACTACAGGTACCTTCTTTTTCACCATCTATTCCTATAGTTTTGAGGGCAC
TACTTACACAAGGGTGGATACGACGGAAACGTCACAAGTTTCCTCAAGTAAGCATGCAAAATTTTGTTTTCAGGTATGAGAGCAC
AGGAATTCGACCAGAATACCTCAGGGATGCAATGCCAATAAGACATGTTCAAAGGAAAATCCAAGATTTTTTATGCAATGGTGAA
CCGATATGGAAGATCCGGCCAAGAAGTGGGAAGGCAAGGATTCTTGTAGGACATGGTCTAGATCATGACATGAAATGCTTGGA
ACTAGAGTATCCTCTTGCAAAAATAAGGCAAGTATCTTCATGTTATTTATCACCTATTTGCGTGATTAACTTAAATATTTATTAATA
ATTTTTCATGCCTCTTTATTACTTTATAGGGATACATCAAAATATCCACCCTTAATGAAGACTAGCAAGCTCAGCAACTCTCTCAA
GTACCTAACAAAAGCATATCTCGGGTAATAATAATGATAATACCACGCTTTAGTAATGTTGAATATCTTTGTTACCCACACATTTTT
AGAGCTAGCTAAAAGATACACATGTTAATGATTAATCGGTTCTTTTACAGCTACGATATTCAAAACGGGATACAAGATCCATACG
ATGATTGTGTTGCAACAATGAGACTATACAGAAGAATGAGATATCAAGCTCATAGAATAGAGGACTATCCTCTTGCAACAGACCC
CCAAAACAAGAACAATTTTACTTCATGGAGACAGGGTGAGCTTGAGAGGATGAGTCCTGATGAACTTTTCGCAATCTCAAGGTC
TGATTACTATTGCTGGTGCTTAGATAGCAAAGACGTGGCTTAA
SEQIDNO: 405
MRTSYHSVHEPICGICGKRSRSFESLREHLIGPLPKAECERVFRERGCDLCLNIFGSRNALRVHRDTCQLSYGYNAVLYRFANLGIQD
DLRIDNGKSRAVALACKMVGGGSDGSLDLCAKICIIDEYENILFRSYVKPYLPVTHYRYETTGIRPEYLRDAMPLRQVQRKIQDFLCNG
EPMWKIRPRGGKARILVGHGLDHDLKCMELEYPAIKLRDTANYPPLMKTSKLSNSLKYLTKAYLGYDIQVGIQDPYEDCVATMRLYRR
MRSQAHRNENYPLASDPQNKNNFASWRQSELERMTPEELLAISRSDFYCWCLDGKDYV
SEQIDNO: 406
ATGAGGACCTCATACCACTCGGTTCATGAGCCCATTTGTGGAATTTGTGGAAAACGCTCTCGATCATTTGAATCTCTTCGAGAAC
ATCTTATAGGACCCTTACCAAAGGCAGAATGTGAAAGGGTGTTTCGCGAGCGTGGTTGTGACTTATGCTTAAACATCTTTGGCA
GTCGCAATGCTCTCAGGGTTCACCGAGATACATGCCAACTATCATATGGATATAATGCTGTACTATACCGATTTGCTAACTTAGG
CATCCAAGATGACCTAAGAATCGATAACGGGAAATCAAGAGCTGTTGCACTTGCTTGCAAGATGGTTGGTGGTGGTAGTGATGG
TTCTTTGGATCTTTGTGCAAAAATTTGCATCATTGATGAATATGAGAATATATTATTCCGGTCCTATGTCAAACCATACCTTCCTGT
TACACACTACAGGTATGAGACAACTGGCATTCGGCCCGAATACCTCAGGGATGCAATGCCACTTAGACAAGTTCAAAGAAAGAT
TCAAGATTTCTTGTGCAATGGAGAACCCATGTGGAAGATCCGCCCAAGAGGTGGGAAAGCAAGGATTCTTGTAGGTCATGGTTT
GGATCATGATCTTAAATGCATGGAGTTGGAGTACCCTGCAATTAAATTAAGAGATACAGCAAACTATCCACCCTTAATGAAGACT
AGCAAGCTCAGCAACTCACTTAAGTATCTTACAAAAGCATACCTCGGGTATGATATTCAAGTTGGGATACAAGATCCTTATGAAG
ATTGCGTTGCAACGATGAGGCTTTACAGGAGGATGAGATCACAGGCTCATAGAAATGAGAACTATCCGCTTGCTTCGGACCCAC
AAAATAAGAATAACTTTGCTTCATGGAGACAAAGTGAGCTTGAGAGGATGACTCCTGAGGAACTGTTGGCAATCTCAAGGTCCG
ATTTCTATTGTTGGTGCCTAGACGGCAAAGACTATGTTTAA
SEQIDNO: 407
ATGAGGACCTCATACCACTCGGTTCATGAGCCCATTTGTGGAATTTGTGGAAAACGCTCTCGATCATTTGAATCTCTTCGAGAAC
ATCTTATAGGTACTGGCTACATGATATAGATTTGTTAAGAGTGATTGCACACCATGAATTTTCACTCACAAATGATCATATCATGT
GTTATAACATGATTTTCTTAGCATCAAGAACATGGCTTACACATACCACTTACTTTTTTTTCACAGGACCCTTACCAAAGGCAGAA
TGTGAAAGGGTGTTTCGCGAGCGTGGTTGTGACTTATGCTTAAACATCTTTGGCAGTCGCAATGCTCTCAGGGTTCACCGAGAT
ACATGCCAACTATCATATGGATATAATGTAATAAATCATCGACCTTGATTGAAAATGCATTTATTTTCTCTGTGTGCTAATCAGGA
AATTAAATCCGCTCCACGCTTATCAATTATTTCAGGCTGTACTATACCGATTTGCTAACTTAGGCATCCAAGATGACCTAAGAATC
GATAACGGGAAATCAAGAGCTGTTGCACTTGCTTGCAAGATGGTTGGTGGTGGTAGTGATGGTTCTTTGGATCTTTGTGCAAAA
ATTTGCATCATTGATGAATATGAGAATATATTATTCCGGTCCTATGTCAAACCATACCTTCCTGTTACACACTACAGGTATTTTATT
CTGCCTTCTTGTACTATATATGTATCTGTGAGTGTGTGTTACTCTATTATGTAAGTATTACACAATAAATTTTGAATTTACATAGGT
ATGAGACAACTGGCATTCGGCCCGAATACCTCAGGGATGCAATGCCACTTAGACAAGTTCAAAGAAAGATTCAAGATTTCTTGT
GCAATGGAGAACCCATGTGGAAGATCCGCCCAAGAGGTGGGAAAGCAAGGATTCTTGTAGGTCATGGTTTGGATCATGATCTT
AAATGCATGGAGTTGGAGTACCCTGCAATTAAATTAAGGCAAGCAAGCTAGCATCTTCTTGTAGACTATCACATGATATAAATAT
GTATGACCATTTATCATGATATATTCTTGATGTTTATAAAGATTTGGTTTTTGTAACATATAGAGATACAGCAAACTATCCACCCTT
AATGAAGACTAGCAAGCTCAGCAACTCACTTAAGTATCTTACAAAAGCATACCTCGGGTAATCTTTGGTAGACCTTGATCATGTT
TATATAGAATTAGTGACTAAAAAGAGCCATAATTTGAAATAATGGATGGACTTTTGATTACACAGGTATGATATTCAAGTTGGGAT
ACAAGATCCTTATGAAGATTGCGTTGCAACGATGAGGCTTTACAGGAGGATGAGATCACAGGCTCATAGAAATGAGAACTATCC
GCTTGCTTCGGACCCACAAAATAAGAATAACTTTGCTTCATGGAGACAAAGTGAGCTTGAGAGGATGACTCCTGAGGAACTGTT
GGCAATCTCAAGGTCCGATTTCTATTGTTGGTGCCTAGACGGCAAAGACTATGTTTAA
SEQIDNO: 408
MDHRYESSETLRNKCSACYRQFNKKEHLVEHMKISYHSVHEPMCGVCRKHCRSFESLREHLIGPLPKAECERIFKDRGCDICLTILAS
RNALRAHRESCQFSLQGLHYRMANLGIQDELRIDNSRGRVVALACKMVGGGSDGSLDLCARVCLIDEHERIIFHSYIKPNLPVTNYRY
ETTGIRPEYLRDAMPVRNVSRKIQEFLCNGEPIWQIRSKGGRSRILVGHGLDHDLKCLEMEYPAIKIRDTAKYPPLMKTSKLSNSLKYL
TKAYLGYDIQIGVQDPYEDCVATMKLYMRMKAQFHKKENYPLATDPQNKNNFASWRQNELERMSPEQLLDFSRSDYYCWCLDTKII
NWRGI
SEQIDNO: 409
CTTCCTCAAATCTTCTCAACTATCTTTCCTCTACTCAAGACTAGAGTTTGAAGAATCTACCACAATTCTACTATAATGGATCATAG
ATATGAGTCCTCCGAGACTCTCAGGAATAAGTGTTCAGCATGCTATAGGCAGTTCAACAAAAAGGAACATCTAGTGGAACACAT
GAAGATATCCTATCATTCAGTTCATGAGCCCATGTGTGGAGTTTGCAGAAAGCATTGCCGTTCTTTTGAATCTTTAAGGGAGCAT
CTTATCGGGCCACTTCCAAAGGCAGAATGTGAAAGAATTTTCAAGGATCGAGGATGTGATATTTGTTTGACAATCCTAGCTAGCC
GGAATGCTCTACGGGCTCATAGAGAATCATGCCAATTCTCTTTGCAGGGATTGCATTATCGCATGGCTAATTTGGGCATTCAAG
ATGAACTAAGGATTGATAATAGCCGAGGAAGAGTAGTTGCACTTGCTTGCAAAATGGTTGGTGGTGGCAGTGATGGCTCTTTGG
ATCTCTGTGCAAGGGTTTGTCTCATTGATGAGCATGAGAGGATCATCTTCCATTCATATATCAAACCAAACCTTCCTGTCACTAA
CTACAGGTATGAAACCACAGGCATAAGACCAGAATATTTGAGGGATGCAATGCCAGTGAGAAATGTGTCAAGAAAAATACAAGA
GTTTCTTTGCAATGGAGAACCTATATGGCAAATCCGTTCTAAAGGAGGAAGGTCTAGGATTCTTGTTGGCCATGGTTTGGATCAT
GATCTTAAATGTCTGGAGATGGAGTATCCTGCAATAAAGATCAGGGATACAGCAAAATACCCTCCACTGATGAAAACAAGCAAG
CTCAGCAACTCTCTCAAATACTTGACCAAAGCTTACCTAGGGTATGATATTCAAATTGGAGTACAAGATCCCTATGAAGACTGTG
TAGCCACAATGAAACTCTACATGCGAATGAAAGCACAATTTCACAAGAAAGAGAACTACCCTCTTGCTACAGATCCACAAAACAA
AAATAACTTTGCATCATGGAGGCAAAATGAACTTGAGAGGATGAGTCCAGAGCAGCTGTTAGATTTCTCAAGGTCTGATTATTAT
TGTTGGTGTTTGGACACAAAGATAATTAATTGGAGAGGTATATGATGAAGAAAGATAGAGAGAAAAGCAACAAGATTTGATATAT
ATATGTTTTGAATTCT
SEQIDNO: 410
TGATTTTTTAAACACAAATACACTGTCTGTCACCTCCTTCTCTAAATCCGCTCGATATCAGACAAACAGGTCGGTAATTATAAATG
AGATTTAAGTCAGGTTTTTGTAAAATAATGATTTTTGAGGTCTTACTTAACGCCAAGACTTTCAAAAAGTGTCTTCAAAGAGACCT
GTATAAATACCTTGCACACCCTCACCTTCTTCCTCAAATCTTCTCAACTATCTTTCCTCTACTCAAGACTAGAGTTTGAAGAATCT
ACCACAATTCTACTATAATGGATCATAGATATGAGTCCTCCGAGACTCTCAGGTACTTAAACTAATACCGTTAATTATTTTCGTTG
AAATTGTGTGACCATGAAAACGCATCTCGTCTATAAGTTTAAACTGTTAGATAGGATGTATAACACACTTTTATTTTATTATGTCTT
TTGACAATTTTTGCAGGAATAAGTGTTCAGCATGCTATAGGCAGTTCAACAAAAAGGAACATCTAGTGGAACACATGAAGATATC
CTATCATTCAGTTCATGAGCCCATGTGTGGAGTTTGCAGAAAGCATTGCCGTTCTTTTGAATCTTTAAGGGAGCATCTTATCGGT
ACATATACCCAAATCTTTCTCAAAAACATGTTAACTTTAATTATCAGTCTACCAATTTATTGGTCATGAATTCTAATTTGTTTGTTTG
ATCAGGGCCACTTCCAAAGGCAGAATGTGAAAGAATTTTCAAGGATCGAGGATGTGATATTTGTTTGACAATCCTAGCTAGCCG
GAATGCTCTACGGGCTCATAGAGAATCATGCCAATCGCGTCCAAATAACGTATGTGTAATAAAACTGTAGAACTATATGATCATT
TCATCTGAAAGTTTAAGGTGTCGGATTTGAAAAGATTAAGCTATCATAAAGAACATTCATTAAAATATTTAAATATGTCTTAAACAA
ACATGTGTGATAACACATGAAGCTATGTTGATTCTCTTTGCAGGGATTGCATTATCGCATGGCTAATTTGGGCATTCAAGATGAA
CTAAGGATTGATAATAGCCGAGGAAGAGTAGTTGCACTTGCTTGCAAAATGGTTGGTGGTGGCAGTGATGGCTCTTTGGATCTC
TGTGCAAGGGTTTGTCTCATTGATGAGCATGAGAGGATCATCTTCCATTCATATATCAAACCAAACCTTCCTGTCACTAACTACA
GGTAATTATTCGAATGATTTAAGTTATATATACTGACAGTCTGTTACACGTACGTTTTAATATGCGATAGCATAAACTTTATTTGTA
ATTATATGAAATATGTGTTTAGGTATGAAACCACAGGCATAAGACCAGAATATTTGAGGGATGCAATGCCAGTGAGAAATGTGTC
AAGAAAAATACAAGAGTTTCTTTGCAATGGAGAACCTATATGGCAAATCCGTTCTAAAGGAGGAAGGTCTAGGATTCTTGTTGGC
CATGGTTTGGATCATGATCTTAAATGTCTGGAGATGGAGTATCCTGCAATAAAGATCAGGTAAACTCTTTGATCCACTCTTTAAA
GACAATTTTATGACTTTATTGTTATAAGTAATACTCCCTCTGTTCCAATTTATGTAGCAGCATTTCCTTTTTAGTCTGTTCCAAAAA
GAACGGCAACATTCTAAATTTGATAACTATTTAACTTCAAAATTACAATTTTGTCATTTTGTCATTGATGAATATAATTCGACCTTCT
TGGTTAGTACTTTCATACTTATAATTATAGAGTTGGTTCTTACTTTTTGTTCAACTTATTTTCTTCTCAAGGGATACAGCAAAATAC
CCTCCACTGATGAAAACAAGCAAGCTCAGCAACTCTCTCAAATACTTGACCAAAGCTTACCTAGGGTAATTGCATAAAACTTTAT
ACTTTTCTTGTCCCCACTAGTAAATAATTTATCGCAACTTTAGAATCTAAGAAAATAAATGGTCATATTAAAATTGTGTAGGTATGA
TATTCAAATTGGAGTACAAGATCCCTATGAAGACTGTGTAGCCACAATGAAACTCTACATGCGAATGAAAGCACAATTTCACAAG
AAAGAGAACTACCCTCTTGCTACAGATCCACAAAACAAAAATAACTTTGCATCATGGAGGCAAAATGAACTTGAGAGGATGAGTC
CAGAGCAGCTGTTAGATTTCTCAAGGTCTGATTATTATTGTTGGTGTTTGGACACACAAGATAATTAATTGGAGAGGTATATGAT
GAAGAAAGATAGAGAGAAAAGCAACAAGATTTGATATATATATGTTTTGAATTCTTAGTCGTAGATATCAAGAATTCAAATGATAT
TCATGAAAGCCAAAATTTAGTACGTAGTATATATTTATTTGCATGTCTACTTTGTAATAGATATAGTTGAAATAAAGATGATTTTCA
CTTGCTACAATGAGTGGGTCAAAGTTTTTTACATGCATAGAAGTTTAACTTTTCTGCCATGCATATATGTGCTCTAC
SEQIDNO: 411
AAAGGTACCCGTGACAGCCGAAGTCAATGAAAGAAAAGAGAAATAATGGGCCCAAGAATCTGGAACATACATAGATCATTACGA
ATGCAATAATGCTATACGCTACCAACAAACTGCACGACAAGATAGAGGAGAGGAAGCATGGCCAAAGAAAAAGAAAAAAGTAGA
GGAGGAATCGGAAATTAATATACTGAATATTGCCTTTAGGAATCGTCCATTTATGCAAGTTGGAGCAATAGTACCTCTACCCTTT
CCCTATAAATATCACCCCACTCCTACCTTTGCGAATATTTTGTATGTTCAGTGAAAGTAAAACAAATTAAATTAACAAGAAACTTAT
AGAAGAAAATTTTTACTATTTAAGAGAAAGAAAAAAATCTATCATTTAATCTGAGTCCTAAAAACTGTTATACTTAACAGTTAACGC
ATGATTTGATGGAGGAGCCATAGATGCAATTCAATCAAACTGAAATTTCTGCAAGAATCTCAAACACGGAGATCTCAAAGTTTGA
AAGAAAATTTATTTCTTCGACTCAAAACAAACTTACGAAATTTAGGTAGAACTTATATACATTATATTGTAATTTTTTGTAACAAAAT
GTTTTTATTATTATTATAGAATTTTACTGGTTAAATTAAAAATGAATAGAAAAGGTGAATTAAGAGGAGAGAGGAGGTAAACATTTT
CTTCTATTTTTTCATATTTTCAGGATAAATTATTGTAAAAGTTTACAAGATTTCCATTTGACTAGTGTAAATGAGGAATATTCTCTAG
TAAGATCATTATTTCATCTACTTCTTTTATCTTCTACCAGTAGAGGAATAAACAATATTTAGCTCCTTTGTAAATACAAATTAATTTT
CGTTCTTGACATCATTCAATTTTAATTTTACGTATAAAATAAAAGATCATACCTATTAGAACGATTAAGGAGAAATACAATTCGAAT
GAGAAGGATGTGCCGTTTGTTATAATAAACAGCCACACGACGTAAACGTAAAATGACCACATGATGGGCCAATAGACATGGACC
GACTACTAATAATAGTAAGTTACATTTTAGGATGGAATAAATATCATACCGACATCAGTTTGAAAGAAAAGGGAAAAAAAGAAAAA
ATAAATAAAAGATATACTACCGACATGAGTTCCAAAAAGCAAGAAAAAAGATCAAGCCGACACAGACACGCGTAGAGAGCAAAA
TGACTTTGACGTCACACCACGAAGACAGACGCTTCATACGTGTCCCTTTATCTCTCTCAGTCTCTCTATAAACTTAGTGAGACCC
TCCTCTGTTTTACTCACAAATATGCAAACTAGAAAACAATCATCAGGAATAAAGGGTTTGATTACTTCTATTGGAAAACCTCAAGT
CCAAGTCTTTTCTATTCTATTTCTTACGTAATAATTATATAGATTAGTATAAAGAACTATGGATTGCAGAATAGAGAGTGCCGAAA
CTCACAGGTTTTCTTAATTTCTTACATCCATGCACAAGCGCACACATGCATACATCACATATGAACGAGTGCATGTGCTTCTATA
CTATTAAACATTGAATTGCATAAAATTAAAAATGAATTATAATCAACAGTGCATGGATATGTATAAATAACAATCATGTTTTTTTTTT
TTTTTTTTGCAATAGGAATAAGTGTGCAGCATGCTTCCGACAGTTCAACAAACTGGAGCATCTTGTGGAGCACAGAATTCTTT
SEQIDNO: 412
GGTACCcgtgacagccgaagtcaatgaaagaaaagagaaataatgggcccaagaatctggaacatacatagatcattacgaatgcaataatgctatacgctaccaacaaactgca
cgacaagatagaggagaggaagcatggccaaagaaaaagaaaaaagtagaggaggaatcggaaattaatatactgaatattgcctttaggaatcgtccatttatgcaagttggagcaa
tagtacctctaccctttccctataaatatcaccccactcctacctttgGGCCTTTGTTTATGCTAAGTGGGATGCTAAAAGTCATCCAAACGGATAAATTGC
TTTTTATTTAAATTTTAAAAATTAAAACTTCTAAGTTTTTGAATTTTAAAATTAATTAACCAAAGTCTTAATTAAAACTGTCCATTAAT
TTAAATTTTAAGAATTTACCAAAATTGTCAACTAAAACCGTACAATTTTTTAATTTTAAAAAATGTGTGAATTTGTTAACTATTTTTTT
AACTTTTAACAATCTACCAATTTGTTAATTTTTAAAAATATACCAATTTATTAGTGCTTATTTTTTAAATTGTAAAATCCTAGCAATTT
GTTAATTAAACCAACGTAAACTAAACAATTGTTATTTAAAAAAAAATATTTTTTTTTAATTTTAAAATTCAAACTATTCCAATTTTCTA
TTGGCTTTTTCTTATACTGTCCAAATTTTATTTTAATTTTAATAAAATAACAATTAACTACAAACCAAATTTTTAAACAATTATTAAGA
GATTCATATTTGTTAATGATTGTATCGGATAAAATAAATTTTTTAACCTTTTTAAAAATCAACATCGTAAATTAATTATTATTATTTAG
AAATAAAAATATCTTTTTAAATTTAAGATTTTATTCCATCCCACAATTTTCTTAGTTTTTTCTTAGATAAATCAGTATTACTTAAATAT
TATCATTTTTTTTTTAAAATACCAAACTATCCCACTACTTCCTTTAATTTTATTTTTATCCTAAAAAAACTTAAAATGTGTTTTTTTTA
AAGAAATATAAAAAATATTTTTCCAACAATAAAATAAATATAAGCTAATGCCTTTTTTTTTAATTTATATAAAATATAATACAAATATG
GATATACGGGCGGGCAGAGCGGCTGTGTTTCCGCTAGTTTTAATAACAGCCTTCCAATTTATTACTTGTATATCATATGGCTATC
AAAACTTGTGCACCGTGTGTGACTACGCTACTTCTACACCCTATAATCCGTCTCCGTCTCCAACTTAAGAAAAAAAAAAAACTAC
AACCTTTATTTTATTTTTCTAATAGTAAAAAATTGTAAATATGGAACAGCAGTGGGCCGCAATAGAACGGGCCCATTGTCCAGAA
AAAAATTGCGAAAATCTTCACCCGTCACCCTTTTTCATTTCATCAAATCCCTCGTTTTCCTTTCCTTTATTCCTATTTCCCACACTT
CTTCTTTCATCATTCATCGCCTTTCACCACGCACGCAGATCCTTTATTCCTATAATCCTTCCTTCGCTCTCTCCAAATTTCGACTC
TTTCGATCAGATCCGCGACACCCACCCCACCAAAACCTTGCGCAACCCATCacctcaagtccaagtcttttctattctatttcttacgtaataattatatagatt
agtataaagaactatggattgcagaatagagagtgccgaaactcacaggttttcttaatttcttacatccatgcacaagcgcacacatgcatacatcacatatgaacgagtgcatgtgcttcta
tactattaaacattgaattgcataaaattaaaaatgaattataatcaacagtgcatggatatgtataaataacaatcatgttttttttttttttttttgcaataggaataagtgtgcagcatgcttccgac
agttcaacaaactggagcatcttgtggagcacaGAATTC
SEQIDNO: 413
TAATTGAAATTTTGATTTTCCTGCACTTTTAAATTCCTGACTTTGTTTTTTATATTTTTAAATTAAGATATTTAATTCTTCAATTTATTA
AAATAAATAATTTTGATGGATCATTAATTTTATCAATTAATTGTTAGTTAATCATTTAAAGTCAAACATTAATGTGACATATTAATAA
TACTTATATAATTACATAAGTTGTTGACATGACACTTAAGTGACAAGAAATAAAATAAAATAAAATAAATAAAAAAAGATTTTGATA
AATATTAAACTCACATTTTATACAAAGATAAGATAATAAATAATTAGGACACTTGTATTATTAGACTTATTTTATTTAGTTAACTTTA
TAAATATTAGTTTCTATATATTATTAGTCAAGAATTATTAATCTTTTTTCAAATTATAAAAATTTATAAATTAAAAAAATTAATATATAA
ATATTTTTCATTTTATTTTATATAATTTTATACATTTTATTTTAATTTTACCAATTTATAATTATATTTCACAAATTAATTTATAAATTAT
TTACATAAATTATTTTGTAAATTATTTACATAACTTATTTTGTAAATTATTTTAATATACATATTATTTTTATTCAAATTATATAAATCA
ATATAATTGTGTTAATTAATATTTAAATTATACAAATTACTTAAACTTATTTTACTATGAAAATATTTTTATTCTAAATATTTAAATTAA
AAAATAATATTTAATTTTTTAGAAAACTTACCTATTAATATTTTAGTGTTCACTATAAGAAGTTATTTAATTTATTAAATAAGTTATTT
TAAAATCAATTATATAAATCCAGAAAATTACATAAATAAATATGTAAATTATTTATATAAATTATTTTATCTAATTTATAGAAAATTTG
TAATTTTATTTTTATATATAAATAATAATTTTTTATTATTTTAATTAAAAAATTATAAATTTTGAACTTTCTAGATTATTTAAATATTATT
TAAAATTTACTCATAATCTATATATTATTTTTAAAATTATAGTAAAATAAGAACAAATTTTTATATAATAATATTTATTCATTTATATTA
AAATAAAATTTTATAAAAAAAAATAAATACATGTAAAATTAAATATTAAATATTTTATTTTAATTTATAAATTGTCATAATTTAATAACT
AATGGGACATATAAAATAGTGTTTATAAAATTAATTAGGCAAAACAAAAATTTTAGTGATATATTGGTTTACCTTTAAATAAGAGGA
TATGAATTAAACTTCTAACCGAATTTATTTTATTTTTTACTTTTATTTAATTCTTTGTTAGATGATATCATGTCAACAAACTCTATAAA
CTTTACCTTAACTTCACTGCACCACATAAACTTTGGATGGTTGAAAGAATCAAAATTGTTAATTCACGCGCGAGAATAACAAGACT
AAATATCTTATTTAGAAATATGGAATCAATTTAGTCTTTTCTTTGTTTTTTTAGTAGGGGAGGGGGGGCATTTATGGACGAGAGTT
TATGGTTGGTTTGTTTGCAACAATATTATGAAATTCATATTAAAACAACGTGACAGCCGAAGTCAATGAAAGAAAAGAGAAATAAT
GGGCCCAAGAATCTGGAACATACATAGATCATTACGAATGCAATAATGCTATACGCTACCAACAAACTGCACGACAAGATAGAG
GAGAGGAAGCATGGCCAAAGAAAAAGAAAAAAGTAGAGGAGGAATCGGAAATTAATATACTGAATATTGCCTTTAGGAATCGTC
CATTTATGCAAGTTGGAGCAATAGTACCTCTACCCTTTCCCTATAAATATCACCCCACTCCTACCTTTGACCTCAAGTCCAAGTCT
TTTCTATTCTATTTCTTACGTAATAATTATATAGATTAGTATAAAGAACT
SEQIDNO: 414
ATCTCTCGACAGTTGCGAACTGAATGCTTAGTTGGTAATGTTATGCCCTATCGCTTTTTGCACCGTCCCATGCATCATTACCCCC
ACACACCTCCATCAACCTCTAAAAAGTTAAGAGTGAAAATTACACACACCCGAGGAGAAGAAAAGCTGCTTCTTCTAAGCAACAC
GACCTAGTTACTTTACTTGTAGGGCCTTTCCCTTTTTCCTAAATTACCCCTCTTTTCATCATATGATAATAATATCCAGCTCAGACT
ATAGAATGATATTATGATAGCATTATAGAGTTGACACTAAAGAGCATTATACATGTTGCACCTGACATTCAAGTTCATAAATACTA
ACACTATGCAATAGATAACATATCCTCAAATAAATGCCACACGTTTGGGTGCTCAAATAACCTCAAAAAGGTTAATAGGATGGGA
TTTAAGAGTCAAGGAATATACCATAGATTTTAACTGTTTTTAAAAATGTATTGTTAAATCTTCAATTATTATTAAATGAGACCCATTA
TAGAAATAAGGAACATAAATTTAGGTTCACCAAACAAAATTAGTACTTAAAAAAATGACACCAATTTAATAATTTAATCAAATAAAA
TGACATTTATTTAATTCATCAATAATAAAAAGTACCGTAATATTATTAAATGTTCAATTAATAGTGTTATCATAATCTGTACATAAAA
AGCAATCACTTTTCTATATAATTGAGAGATTAAATTAATATTTGTTTAAACTATCACTGAATCCATTTACCGACCTTTTATATGCCAT
TTTTTATATATATTTGTATGCTTTAAAATCACGGAAAAAAAATTATAGAAAATTCTAACATGATCATTTCAATCATCAATTTAAACAA
CTCTAAATCGAAAACATTGAATAAACTAGAAATGATACCTGGGAATTTGGAACAAGAGACATTCCGCAAAAGAAAAAATATGTTG
AATAATTGAAACGGTGACAAGAAAAGAGGAATAATGAAAATCAATAAAACAGAGATGGGGGGTTATTGTTATTCGAGGAGACGA
GTGAAATGAGTGAGTGAGGGGGGTGTAACTGGAAAGCAAGAAAAAGCACAAGAGTGCCAGCTATTTCCAACAACAAACGTGGC
CCGTGGATGCGATATTGGTAACGGACGGCGAGGATGGAAGGACGTGCAATTTGCGCTTCATTTGAGGCGAATTTCATTTGACC
AGACCTTTCTTTTTTAAACCACAGGGCGGGTACCTCAGCCACACACATACATCATCAAAAAACACACACGACACACTCCCATACA
CACACGTTGTCTCGCACACATTCCTTCATTCCGCAGGTATAGCCACGTTCTCTCTTCCCTCTTTCATCCCCGGTGATAAGAACAA
GCACCCACCGTTTAACGCTAATTTTATGTGTTTTTGTTTTTTTTTTTTTTTTGCAGCAGCAAACATCTTTACCTTAAACGACAACA
SEQIDNO: 415
GAAACAATGCCTAAACATTGTACTAAAATTCACATCAAGCAAAATACAATTTGGTGAACATTAATTGTACCATAAAACATCTATGTT
TGAATAAAGATATACATCCTCAACCTAAATTTACAAGCTAATATAAAATAATAAAATAAAAAACAAATACATAAAACTGCATAAACG
TCTCTTAATCTCGATCGTGTGAGTAAAATGGAACATGTATAAATGTTACATGTTATCCTCACTATCATTCGCTATGACTAATAGTC
TAATACTAATACTAATAATGATAATAATGCTTGTTTTTAAAAATAGTAATAATAAAACAAGTTCACAAGTTAGGTCAATGAGTGCAT
AAATGAAAACCTGTGACCCAAATCCAAAGTCAACGAATTTGAGGATTTTGCTTCTTGCATTTCTTTTTTTTTTCTTCCTGCACTTCC
AAAAATACTCGAATGGACACAACTACCCTTACAAGCACCAACCAAAATTCAACACAAATCGATAACGCCTACTCCCACCAAAATG
GAAACCATTTTCACCAACAACAACAAGGTTCGAAAGGTAGTAACAAGGAAGAGAAAAGGAAAAGGGTTGCAATGGAGATGTCCG
AAGTAGAAGAATAAAACACTATCAATGTAACCCACTACTTTGTTCTTGCTAACATGCATCAACAAACTAAACTAAATGCAATGTAC
AATACATGGCAAAAAATAATGTCAAATCATAAGAAATTCAAGCACAAATATTGCCCTAAAAATTAAATTCAACAAAGAAGTTTCTAT
ATTGCAGTAATAATTTTTCACTTTGCCCCCTCCTCTTAGACTATGCGCGGTGGACCAAAGCTAAAGTTAGAAGGGCTAGGCAAG
ACAAAAGCATTATGTTGAGGTCCCAAATTAGGATAAAAGGAAAAGTGAGAAGGAAGCAAGGAGTGTCACCCTATTTTGAAGCAG
CTATTATGGAATGTGAAAATTAGATTAAGAAATATGTATTCTGGAATAGGTTTATAGATTTCTTATCTTTATTCACTAATAACTATTT
CAAAATGTAAAAATTATATATTTATCTAAACCCACTAGATACTCAATCCAAACTAATTGACTTGATTAAGACGGAATTTGAATTCGT
GGGAAATTCATACTAGGTTTGGGGCAAGGGGTGGAATAAGAAGAAAAAAATGGATAAGGATGTCATTTGGTGTGGGTATATAGG
CAACCAGAAAAGGGCTGGGTATTTTTGAGTGTGTGAGAGAGCGAGCGAGCGAGCAGTTTCAAAGAGAGGATTTGGGATTGGGA
AGGCGAGGGATTGCGCTGCGTCTCTTCCTCTTCTCTTTCGATTCATCCCTCATTCATAATTTTCCAATCCAACAACCTCAGCCTC
ATACTCACACATACACACGTCTCTCTCTCTCTACTCTTTTTTCCTCTCTATTTCCCTTTTCACTTTCTCTTTCATTTCTAGGGTTCC
GTTCTGATCCCTCTTTCCCTTTCAGATCTGAA
SEQIDNO: 416
ATGGCGTCTTCGTCGTTCTCAGTCACATCTCCAGCTGCTGCTGCTTCCGTCTATGCAGTCACTCAAACCTCCTCGCACTTCCCA
ATCCAAAACCGCTCTCGCAGAGTTTCTTTCCGTCTCTCTGCTAAGCCCAAACTTCGCTTTCTCTCCAAGCCTAGTCGCAGTAGCT
ACCCTGTGGTGAAAGCA
SEQIDNO: 417
MASSSFSVTSPAAAASVYAVTQTSSHFPIQNRSRRVSFRLSAKPKLRFLSKPSRSSYPVVKAG
SEQIDNO: 418
MASSSFSVTSPAAAASVYAVTQTSSHFPIQNRSRRVSFRLSAKPKLRFLSKPSRSSYPVVKAGMLHGASSRPATARKSSGLSGTVRI
PGDKSISHRSFMFGGLASGETRITGLLEGEDVINTGKAMQAMGARIRKEGDTWIIDGVGNGGLLAPEAPLDFGNAATGCRLTMGLVG
VYDFDSTFIGDASLTKRPMGRVLNPLREMGVQVKSEDGDRLPVTLRGPKTPTPITYRVPMASAQVKSAVLLAGLNTPGITTVIEPIMTR
DHTEKMLQGFGANLTVETDADGVRTIRLEGRGKLTGQVIDVPGDPSSTAFPLVAALLVPGSDVTILNVLMNPTRTGLILTLQEMGADIE
VINPRLAGGEDVADLRVRSSTLKGVTVPEDRAPSMIDEYPILAVAAAFAEGATVMNGLEELRVKESDRLSAVANGLKLNGVDCDEGET
SLVVRGRPDGKGLGNASGAAVATHLDHRIAMSFLVMGLVSENPVTVDDATMIATSFPEFMDLMAGLGAKIELSDTKAA
SEQIDNO: 419
GATATCATCTTCTCTCTCTCTTTCTGAAGTGAACTGTTGTTGTTGTGCTCTTTGGTTTCATCGAGAGAGAACCTAAACCCGATCTG
AACCAATAACAATAAGAAGACGA
SEQIDNO: 420
ACAATAAACTCTGTTTTTTCTTCTGATCCAAGCATATCTGTTTCCATTTTTCTTGTCTCTGTAATATTATTAGAAACCGAGAGTGTT
TGTTTGCGTGTAACTGAACTGAGCGAGTTTTGAGATGCAATCATTTGAGTTCGATTGAGAGAAATGAATGTGTAGGGCCCA
SEQIDNO: 421
GATATCATCTTCTCTCTCTCTTTCTGAAGTGAACTGTTGTTGTTGTGCTCTTTGGTTTCATCGAGAGAGAACCTAAACCCGATCTG
AACCAATAACAATAAGAAGACGAATGGCGTCTTCGTCGTTCTCAGTCACATCTCCAGCTGCTGCTGCTTCCGTCTATGCAGTCA
CTCAAACCTCCTCGCACTTCCCAATCCAAAACCGCTCTCGCAGAGTTTCTTTCCGTCTCTCTGCTAAGCCCAAACTTCGCTTTCT
CTCCAAGCCTAGTCGCAGTAGCTACCCTGTGGTGAAAGCAGGCATGCTTCATGGTGCAAGCAGCCGTCCAGCAACCGCTCGCA
AATCCTCTGGACTTTCTGGAACCGTTCGCATTCCAGGAGACAAGTCTATCTCTCATCGTTCTTTCATGTTCGGAGGTCTCGCTAG
TGGTGAAACTCGTATCACTGGACTTCTGGAAGGTGAGGACGTTATTAATACTGGTAAGGCAATGCAAGCTATGGGTGCTAGAAT
CCGTAAGGAAGGAGACACCTGGATCATTGATGGAGTTGGTAATGGAGGTCTTCTTGCACCTGAGGCACCACTCGATTTCGGTA
ATGCTGCTACAGGATGCCGTCTTACCATGGGACTTGTTGGTGTTTACGATTTCGACAGCACCTTCATTGGAGATGCATCTCTCA
CAAAGCGTCCTATGGGACGAGTGTTGAACCCACTTCGTGAAATGGGAGTGCAGGTGAAATCTGAAGACGGTGACCGTCTTCCA
GTTACCTTGCGTGGACCTAAGACTCCTACTCCAATCACCTACCGTGTGCCAATGGCATCTGCACAGGTGAAGTCAGCTGTGCTG
CTCGCAGGTCTCAACACTCCAGGTATCACTACAGTCATCGAGCCTATCATGACTCGTGATCATACTGAAAAGATGCTGCAGGGA
TTTGGAGCTAACCTTACCGTTGAGACTGATGCTGACGGAGTGCGCACCATCCGTCTGGAAGGTCGTGGTAAGCTCACCGGTCA
AGTCATCGACGTGCCTGGAGACCCATCCTCTACTGCATTCCCACTGGTTGCAGCTCTGCTTGTTCCAGGCTCCGACGTCACCAT
CCTCAACGTGCTGATGAACCCTACTCGCACTGGACTCATCCTGACTCTGCAGGAAATGGGTGCTGACATCGAAGTCATCAACC
CTCGTCTTGCAGGAGGTGAAGACGTGGCTGACCTGCGCGTTCGCTCATCCACTCTGAAGGGTGTCACAGTGCCTGAAGACCG
CGCTCCTTCTATGATCGACGAATATCCTATTCTCGCTGTTGCAGCTGCCTTCGCTGAAGGTGCTACCGTGATGAACGGTCTGGA
AGAACTTCGCGTCAAGGAAAGCGACCGTCTCTCAGCTGTCGCCAATGGTCTCAAGCTCAATGGAGTGGATTGCGATGAGGGTG
AGACTTCACTCGTCGTGCGTGGACGTCCTGACGGTAAGGGACTCGGTAACGCATCTGGAGCTGCAGTCGCTACCCATCTCGAT
CACCGCATCGCTATGAGCTTCCTCGTCATGGGACTCGTGTCTGAAAACCCTGTCACAGTGGACGATGCCACGATGATCGCCAC
GAGCTTCCCAGAGTTCATGGACCTGATGGCTGGACTTGGAGCTAAGATCGAACTCTCCGATACGAAGGCTGCCTGATGAACAA
TAAACTCTGTTTTTTCTTCTGATCCAAGCATATCTGTTTCCATTTTTCTTGTCTCTGTAATATTATTAGAAACCGAGAGTGTTTGTTT
GCGTGTAACTGAACTGAGCGAGTTTTGAGATGCAATCATTTGAGTTCGATTGAGAGAAATGAATGTGTA
SEQIDNO: 422
TCAAAAAAATTTCTGGTTATGCATCTCTTTCTTGAAAAAATAATCATATAATCACATCAAAAAGAGTCAATTATCATCTTCATTATCT
TAATATAATCAATCAATAATTCAATTTAAAAGGTAAATTAAAGTAAGCAATGTAGACCCAAAAAAAAAAGTCTGCTTTTGTTTCAAA
ACATATAAGGACAGTGAAAGGGTTTGTGCATAAATGTTGTTGCTTCATCTTTCTCATAACATTAGCTATGAGATTATGAAAGTGAA
GATATAAAACAAACACCTAAAAGTGAAAAGTACAAGACACCAAATCCTTCATTTTCATCTCTTACACAACCACACTAAGTTACCAA
AAAACAGAATCTGTTCTTTTTTTGCCTTCTTGCAGATTTCTTCAGAGTCAAAGACACATAAAGATTTATAATTGAGTTGTGAGTTTT
GGAGGCCATGGACGTAGGAGAGAGCAATGAGAGGGTAAAAGACGATTCTGCCCTTCAAGCTTCACCGAGGAGTCCTCTGAGTT
CTATAGATCTTGCAATTGATGGAGCTATGAATGCTTCTATTGAGCAGTTGTATCATAACGTTTGTGAAATGGAGAGTTCTGATGAT
CAGTCTCCGTCCAGGGCGAGTTTTATTTCGTATGGAGCTGAATCTAGGATTGATTTGGAGTTGAGGCATTTGGTTGGAGATGTA
GGAGAAGAAGGAGAGAGCAAGAAAGAGATTATCTTGGAGAAGAAGGAAGAAAGTAATGGAGAAGGTAGCTTGAGTCAGAAGAA
ACCTCTTTCGAATGGTAAGAAGGTTGCGAAAACGAGTCCTAATAATCCGAAGATGCCGGGTTCGAGGATCTCTTCGAGGAAATC
ACCGGATTTGGGGAAGGTTTCTGTTGATGAGGAGAGTCCTGAGCTTGGTGTGGTGTTGTTGAAGCAAGCCAGAGAGTTGGTTT
CTTCAGGTGAGAATTTAAACAAGGCTCTTGATTTAGCGTTGAGAGCGGTGAAAGTGTTTGAGAAATGTGGGGAAGGAGAGAAAC
AACTTGGGTTGAATTTGGTGATGTCTTTGCATATCTTGGCAGCGATATATGCAGGGTTAGGGAGATACAATGACGCTGTGCCTG
TTCTTGAACGTTCTATCGAGATACCGATGATTGAAGATGGTGAGGATCACGCGTTGGCGAAATTTGCAGGGTGTATGCAGCTTG
GTGACATGTATGGTTTGATGGGTCAAGTTGAAAATTCGATTATGCTATACACCGCGGGTTTGGAGATTCAAAGACAAGTTCTTGG
AGAATCGGATGCACGAGTTGGCGAAACTTGTAGGTACTTAGCGGAAGCTCATGTTCAAGCAATGCAGTTTGAAGAAGCCTCAAG
GCTTTGTCAAATGGCATTGGACATTCACAAAGAAAACGGTGCTGCCGCTACAGCTTCTATTGAAGAAGCTGCGGATAGAAAACT
GATGGGACTTATCTGCGATGCGAAAGGTGATTATGAAGTTGCGCTTGAGCATTATGTTTTAGCGAGCATGGCAATGTCATCTCA
GAACCATAGAGAAGATGTTGCTGCTGTAGATTGCAGTATTGGTGATGCTTACATGTCGCTAGCTAGGTTTGATGAAGCGATATT
CGCATACCAGAAGGCTTTAGCTGTGTTTAAGCAAGGAAAAGGTGAGACTCATTCCTCTGTTGCTTTGGTTTACGTTAGGCTTGCT
GACTTGTACAACAAGATTGGGAAAACACGCGATTCCAAATCGTACTGCGAAAACGCTCTCAAGATTTACCTAAAACCGACTCCA
GGAACTCCTATGGAAGAGGTTGCGACTGGTTTTATAGAGATTTCTGCGATATATCAGTCAATGAATGAACTTGATCAAGCTCTTA
AGTTGTTGAGACGGGCGTTGAAGATATATGCAAACGCTCCAGGTCAACAGAACACGATTGCAGGTATTGAAGCTCAAATGGGT
GTGGTTACTTATATGATGGGAAATTACTCCGAGTCCTACGACATATTCAAGAGCGCGATATCAAAGTTCCGCAATAGCGGAGAG
AAAAAAACCGCTCTTTTCGGGATTGCTTTGAATCAGATGGGACTAGCTTGCGTTCAGCGTTACGCGATCAATGAAGCTGCAGAT
TTGTTTGAAGAAGCGAAAACTATTCTTGAGAAAGAGTGTGGACCGTACCATCCCGATACATTAGCGGTTTACAGTAACCTTGCTG
GAACGTACGACGCAATGGGAAGGTTAGATGATGCTATAGAGATATTGGAGTATGTTGTTGGGACAAGAGAAGAGAAGCTCGGG
ACGGCAAATCCCGAGGTTGAGGACGAGAAGCAGAGGCTTGCTGCGTTGTTGAAAGAAGCTGGAAGAGGAAGGAGTAAAAGAA
ACAGAGCACTTCTTACCCTTTTGGACAACAACCCTGAAATTGCAAATGGACAGAGACCGGTTTATTGATTAACCGGATTATTGTC
CGGTCTAAAATGTATATATATACAAAATACAAGTTCGAATACGGTACTTACACGGTCGAGGGAGAAGGAAAAAAATGAAGAGGC
TTTATGTTTTCTAGTTTTATAAAAAGTGAAGGAAAAAAAAAAAAATCTTGCATTTGTTTTCTTTATAAATAAAATGTAAAGAGTAAAA
CGCAATTGATTTGGTCGTTTTGTAATGTGATTTACATTGAAATTTTGGAGTTTAAAAATCTCCTTGATGGCAGTTTCTCTAACTTTG
AGCACTG
SEQIDNO: 423
MDVGESNERVKDDSALQASPRSPLSSIDLAIDGAMNASIEQLYHNVCEMESSDDQSPSRASFISYGAESRIDLELRHLVGDVGEEGE
SKKEIILEKKEESNGEGSLSQKKPLSNGKKVAKTSPNNPKMPGSRISSRKSPDLGKVSVDEESPELGVVLLKQARELVSSGENLNKAL
DLALRAVKVFEKCGEGEKQLGLNLVMSLHILAAIYAGLGRYNDAVPVLERSIEIPMIEDGEDHALAKFAGCMQLGDMYGLMGQVENSI
MLYTAGLEIQRQVLGESDARVGETCRYLAEAHVQAMQFEEASRLCQMALDIHKENGAAATASIEEAADRKLMGLICDAKGDYEVALE
HYVLASMAMSSQNHREDVAAVDCSIGDAYMSLARFDEAIFAYQKALAVFKQGKGETHSSVALVYVRLADLYNKIGKTRDSKSYCENA
LKIYLKPTPGTPMEEVATGFIEISAIYQSMNELDQALKLLRRALKIYANAPGQQNTIAGIEAQMGVVTYMMGNYSESYDIFKSAISKFRN
SGEKKTALFGIALNQMGLACVQRYAINEAADLFEEAKTILEKECGPYHPDTLAVYSNLAGTYDAMGRLDDAIEILEYVVGTREEKLGTA
NPEVEDEKQRLAALLKEAGRGRSKRNRALLTLLDNNPEIANGQRPVY
SEQIDNO: 424
ATGGCCGCCGCAGTTTCCACCGTCGGTGCCATCAACAGAGCTCCGTTGAGCTTGAACGGGTCAGGATCAGGAGCTGTATCAG
CCCCAGCTTCAACCTTCTTGGGAAAGAAAGTTGTAACTGTGTCGAGATTCGCACAGAGCAACAAGAAGAGCAACGGATCATTCA
AGGTGTTG
SEQIDNO: 425
MAAAVSTVGAINRAPLSLNGSGSGAVSAPASTFLGKKVVTVSRFAQSNKKSNGSFKVLG
SEQIDNO: 426
TTTCATGGTCCGTAGACTTTTATCAGGTTTTTAATTAGGTTTCTGAATTGAAATTATATCTTATTTAGTATATATAAAGAAAATAATT
ATTTTAACCTATTATATCAAAACAGTGAATCCTTTAACTCTAAAACTAATCCTATCTTTATTTTACAAAAAATACTAAACCTATCTAT
TATTTTTTTTAAATGTTAAACCCATCATCTAGGATAGTTTTAAATTCGCATAAAAAAAGATAGTTTTAAATTAGATAGGATTTTTTTT
ATATCAAAACATTGAATCCTTAAACTCTATAACTAATCCTATCTTTATTTTTAAAAAAATACTAAACCTATCTAATATTTTTTTAAAG
GATAAACCTATCTAATCTAAGATAGTTTTAAATTAGATAGGATTTTTTTATATCAAAACAGTGAATCCTTAAACTCTAAAATTAATCC
TATCTTTATTTAAAACTATCTAATCTTTTTTTTAAGGATAAACCTATCTAATCTAAGATAGTTTTAAATTAGATAGGTTTAGTAAACC
TATATGAATTTTATTTTTTTATGATTAAAAAAAAATCTTAGATATGATTTTATTTTAATCCCAGCATTTGCCACGTTCGAACGTGAGC
CAAAACGATGTCGTTACAATATCTTAACCTAGCCGAAACGATGTCGTGGTAACTCATAATATCGCAGCCTTCTGTTTCGTCCTTG
CCCAATGCCAACTGGACTACGCCGAACCCACAAAACCCGACATCCGCGCGAACCAAAGCGCGTGACGCCAAATTGCTCAAACC
ACAAAGGAAACAAAGGCGCGTTAATAACACGCGCTATCCCACGCGTGTTAAGCACAGTAACGTTCAACAAAGAGAAGCAGGGT
ATAATGGGAAACCAAAGAGAAACCTAAGGCCATTATGGGAAACAAAAAGTAGCTCACTCATTATAAAATTCAAGTAACCCTAGTT
TTCATCATCACTCTGCTGCCTCGCTCTGTTTCTGTCTCTGTGTTTGCGGCTGAGGATTCCGAACGAGCGACCTTCTTCGTTTCTC
GCAAAGGTAACAGCCTCTGCTCTTGTCTCTTCCATTCGATCCATGCCTGTCTCTTCTTTACGATGATGTTTCTTCGGTGTATGTTT
ATTTATTTATTTATTTATGCTTTATGTTGTGAATGTTCGGTTGTTTTGTTTCGCTTTGCTTTTGTGGATTCTATTGGTTTTTGAATCA
GTTAATCGGAAGAGATTTTCGAGTTGTTTTGTGTTTTGGAGGTGAATCTTTTTGTTGAGGTCGCAGATCTGTTGATTTTGTGTCAT
AAACGTGCGACTCTGTTTGATTTTTTACGAGGTTATGACGTTTTGGTTGTTTTATTATGGATCTGTTAAGGCAGAACCATGATTTA
TGTTTATGTTCGTTTACACGATTAAATTTTCTTGTAACACGATGAAGTTTTTTTAAACACGTTGAAGGAGTCTTGTTGATATGAATT
TGTCGATTGTTTTTTTGTGGTTTTGTTCTCACGTTATCAAGCGTAATCTTTTACTATGTACGCGAACATATCTAGATCTAGCAGAG
CTTTTTTTTTTTTTAATTCTTTGTGAAGCTTTTGAAATATGAAATTTGTTTTTCAAATTTTTTTTAATTTATTGAAAACACTGTGGATA
CTGAATAAGGTTCTATGTATGATGCGAACCATGTTTGATATGTTTGTTTTTGTTGTTCCATATGGATTTTTCTGTTAGATTTCCATA
TGCTTTTGAGTTTGTTTTTGCTGTTACAGATTGATTTACTTTTTAGAATATTTCTCTTGGCTTTCACTGTTTTAGAAATTTTTTGTTA
TTGGTAACTATAAATGTGTGAATTTGGATTATACCTTTACCTTGTTATTTAGTTGTGTGATAATTTGGTTTATAGTTATTTTGAGTTC
TGACTCGTGTTTCTTTGAATTGATTCCAGTTTAAGTCATCTCGAG
SEQIDNO: 427
AGAATTTTACTTAAATCTCTACTACATAAAATAATAAAAATATAATATATTGCATGATATTTAGTAACTGTAAAAAAAAATATAAATA
TTACATGATATTTAATAACTGTAAACAAATAAAAATATAATATATTGCATGATATTTAGTAATAGTAAACTAAATAATAAAAAATTAT
GACATGGAAACTTTTATAAAAAAAGAAATACTATAATTTTTAAGATAGACAATGATAGGAAAAAAAATTAAAAAGTTTCAACGACTA
CACATTTAAAATTTAAAATTAATAATTGCTTTCTACAAAATGAGAATATATTAGTTTACAAAAGTAAAATAGTTACCAAATTTATTGA
AAAAACACATTTTTTTTATTCAAAATTATATTTTTCTTTTTTTAGTTTTATATTAATAATAACAAATCAAAATTAGTGATTTATCATTAT
TGAGAATAAAAAAGTTGTTTTAATATAATTATATTTATTATGTTATAAATTTAAATTTGTTTAATACTTTTTTTATTATAATTAAATAGA
AAAAATAAAAATTAAATTACATTACTTTTTAAAGTTCTTTTACCAACTCGATGAAATTATTATATTTGACAAAATTATTTTTAATATTT
GATGCAAATTTATTTTTTTGCAAAATTAATTAATTAAAGAAAAATACATTAAAATAAAGATAATACATAAATATAAAAATGATATGAT
GTAAAACTAATCTATTATACCATACTTAAAAGATTTGTTTTGGAAACTGCACTAAACTACGTCGTTTTGGTGGGCGCATAATTAAA
CCCTAACCAGGGCTTCATCACGACACTCTCGGGGCCTCCCGCACAGCAAGCGAAAGCAAAAAGGATCCAAATGGAACTACTTA
AAATAATAAGGGTATTTTCGTCAACAGAGGAAAAAACTTGTAGCCTATAAATAAACCCTCTAACCCTCGTTTCCTCACTTCTCTTC
ACTCTATCATTCTCCTTTCTTCTCTTGCGGCTAGGGTTTTAGCGCAGCTTCTTCTAGGTACGTTTTCTTATGCCGTTTCATTAGTT
TTAATTCTTCTATTCATGCTTATCCTATTGGATTGTGTTGCCTGCTGAATATGGTTTTCTATATGTTGTTTATGTCTTCAATATATCT
CTGTTTTGTTGATGAATTTTAATTGTTTCACATGAATTTCATGTTTTTTTCGAATTTTTGTCTGAGTTATTGGTTATAATCGGTAAGA
TGTCTTCAAAAAATGATTTTTTTTTTCGTTTGGTTTTGTACCTTTGAAATACTCAGATTTGATTTGATTTTTGTGGATTTGTGTGTTT
TTTATATTCAAATCCATGGATCCAGATCTGGTTAAACTTTTGCTTTTTGTTTAAGATTTTTCTCAATTCTGATGGATGGATCTATAA
CCTTTTGGTTTTAATCGATTCAAGTCGCATTTTTTTTTTGAGATGGCATGATAAATGTGTGGTTTAACCCACTGGTTTTAATTGGTT
CAAGTTGCGGATTTTACGGGATGACATGATAATTGTTTGGTTTAACCTTTTATTTTAATCGATTCAAGTCGCTGATTTTACGAGAT
GACATGATAAATGTGTGGTTTAACCTTTTGGTTTTAATCGATTCAAGTTGCTGACTTTACGAGATAGACATATAAATGCGTGGGTG
TCTTGAATACCTTTTTCGGATGTGGTGTAGTAACATATGTGTGGGTGACTTGAATACCTTGTTTGCATGTGGGTGTCTTGAATAC
CTTGTTTTTGTGGTGTAGTAACTTATGTGTGGGTGTCTTGAATACATTGTTTGCATGTGGCGTAGTAACTTAATTTTGACGTGTCA
ATGCGTCAATATTTTTATTTCTGTATGTAATCTGTTTTGTGTTCCCTTTGCTCTAATGAATTATACCTACTAATAGCTTGTTGTCTG
CGTTGCTTATAGTTGTATGCCTGCTTTATATGAAGTTTGTGTGCAAGGAATTTGGAGATTACCCGATATTTTAATGTTTTAATGGT
TATACATGTCTTGTAATATAAAGCTTGTTGTGTGATTTCTTCTTATATATTGCCGTTACTTTTAATTGGTATGCATAATGTAAACTTT
GTTTTTAGATATTACCCAATATATTATTTATGATTATCCATGGTTTGTTGTATATGCGAACCCATCTCCTTTGTGTGATGTGGCTAT
TTTTTGGTTTAAGATGATGCTAATGTATGCATTTAGTTGTTGCTTTTTGCAGATACTCAAACGCAGTT
SEQIDNO: 428
AATCTACTTCAGATTCTTGTTCTTGTGGTCTTTTTCTGTTGTTGTGTTTGATGTTCAGATGTACCAGTACCGTTGTGGGTTTTAATT
TGGATGCATTGTTTTTATGCTTCGCTGCTTCTGTTACAGACCCAAACGGAATGGCAATATATATTTTGACCATTGTCTGAACTACT
ATTTTGTTTTAGCTGTGTTAATTATTGACTTTTTTTCTTTTCGATTAAGTGTGGTACTTTTTCGGAACTTAAGGGCCAAAAATTTGA
TTGTAGAGGAGAGGAGTTTTGAGATCCCCTTCCTTTAGGAAATGAGAAAAAAAAATAGAAAAGAAACGAGATTTTTAAAATTCTTA
CTAGT
SEQIDNO: 429
AACAATGAGCCCAGAAAGAAGACCTGCTGATATTAGACGTGCTACTGAAGCTGACATGCCAGCAGTCTGTACTATTGTTAACCA
TTACATTGAGACTTCTACTGTTAACTTCAGAACTGAACCACAAGAACCTCAAGAGTGGACTGATGATCTTGTTAGACTTAGAGAG
AGATATCCATGGCTTGTTGCTGAAGTTGATGGAGAAGTTGCTGGAATTGCTTATGCTGGACCATGGAAGGCTAGAAACGCTTAT
GATTGGACTGCTGAATCTACTGTTTACGTGTCTCCAAGACATCAAAGAACTGGACTTGGATCTACTCTTTACACTCATCTTCTTAA
GTCTCTTGAAGCTCAAGGATTCAAGTCTGTTGTTGCTGTTATTGGACTTCCAAACGATCCATCTGTTAGAATGCATGAAGCTCTT
GGATATGCTCCAAGAGGAATGCTTAGAGCTGCTGGATTCAAGCATGGAAACTGGCATGATGTTGGATTCTGGCAACTTGATTTC
TCTCTTCCAGTTCCACCAAGACCAGTTCTTCCAGTTACTGAGATTTGATTCGAA
SEQIDNO: 430
MSPERRPADIRRATEADMPAVCTIVNHYIETSTVNFRTEPQEPQEWTDDLVRLRERYPWLVAEVDGEVAGIAYAGPWKARNAYDWT
AESTVYVSPRHQRTGLGSTLYTHLLKSLEAQGFKSVVAVIGLPNDPSVRMHEALGYAPRGMLRAAGFKHGNWHDVGFWQLDFSLPV
PPRPVLPVTEI
SEQIDNO: 431
GGTTTGAGTTTGAGTTCACATATGGGAAAATAGGATTGTCAATTCTAGTTCTTTGTTTCTCTAATGTGTCTCAGTTTATAAGATTTA
GTTTGTTTTCATTTTTCAATTGTATATTTGTGGGGTCAGGAATTTTGATCTCTGTACGAAGTAAATTATGTGAGGAACATCTTTTGC
ATGATTATGCTTTTACAAATTTGGGATGGATGCAATGTCATTCTGAACATGATTTCGTTTTTTTTTAATAATCAACATCATATATATT
GAACAAGGCTTTTAAAACAGAACCCGTGTGAAGGTATATATACAAGATTATATAATAATTAAG
SEQIDNO: 432
CTAGTGGGTGAATAATCATGTATTGTGTATTGTGAGATTGTAGAGTTCAAAATAAAGATTATACATAATTATGAGTGGTTATATAG
GATATTATTTTTTTTTGCTTACTTTTTTTCCCCTTCAATTTGTATCCTTTTATTATAGTTGAAAAAATATAATAAGAAATTTTAAAATA
ATTAAAGGATAAAGAAGTCTATGTAAAAGTTGATATCGAAAATAATTTTCAAATCAGGTATCTCCTTTATCTATTGGTATAAATTAT
GTTGATATTTTTTCATTTTTGTTATCATTGGCCTTTGTTTATGCTAAGTGGGATGCTAAAAGTCATCCAAACGGATAAATTGCTTTT
TATTTAAATTTTAAAAATTAAAACTTCTAAGTTTTTGAATTTTAAAATTAATTAACCAAAGTCTTAATTAAAACTGTCCATTAATTTAA
ATTTTAAGAATTTACCAAAATTGTCAACTAAAACCGTACAATTTTTTAATTTTAAAAAATGTGTGAATTTGTTAACTATTTTTTTAACT
TTTAACAATCTACCAATTTGTTAATTTTTAAAAATATACCAATTTATTAGTGCTTATTTTTTAAATTGTAAAATCCTAGCAATTTGTTA
ATTAAACCAACGTAAACTAAACAATTGTTATTTAAAAAAAAATATTTTTTTTTAATTTTAAAATTCAAACTATTCCAATTTTCTATTGG
CTTTTTCTTATACTGTCCAAATTTTATTTTAATTTTAATAAAATAACAATTAACTACAAACCAAATTTTTAAACAATTATTAAGAGATT
CATATTTGTTAATGATTGTATCGGATAAAATAAATTTTTTAACCTTTTTAAAAATCAACATCGTAAATTAATTATTATTATTTAGAAAT
AAAAATATCTTTTTAAATTTAAGATTTTATTCCATCCCACAATTTTCTTAGTTTTTTCTTAGATAAATCAGTATTACTTAAATATTATC
ATTTTTTTTTTAAAATACCAAACTATCCCACTACTTCCTTTAATTTTATTTTTATCCTAAAAAAACTTAAAATGTGTTTTTTTTAAAGA
AATATAAAAAATATTTTTCCAACAATAAAATAAATATAAGCTAATGCCTTTTTTTTTAATTTATATAAAATATAATACAAATATGGATA
TACGGGCGGGCAGAGCGGCTGTGTTTCCGCTAGTTTTAATAACAGCCTTCCAATTTATTACTTGTATATCATATGGCTATCAAAA
CTTGTGCACCGTGTGTGACTACGCTACTTCTACACCCTATAATCCGTCTCCGTCTCCAACTTAAGAAAAAAAAAAAACTACAACC
TTTATTTTATTTTTCTAATAGTAAAAAATTGTAAATATGGAACAGCAGTGGGCCGCAATAGAAT
SEQIDNO: 433
GGATCCGATTGTTTTCCATTTGTTTCTGAATTTTGGAATTTTATTTTAGTATAATACTGTTTTTTTTTGGGGTCGCATATTTATCATT
ATGACTGCCTAAATGATCTGCCTTTTCATATTATTATGACTGCTTAAATCTTGATGCATTATTTTTTGAATTTGATTTTAATTTTGAA
ATGAAGTGAGTGATTTATGGCTGAATTTAAGAAATTTGATGGAAATTTATAGGTAAAATCAGTTTTACTTTATCGATAACTTAATGT
TAGC
SEQIDNO: 434
MDNNPNINECIPYNCLSNPEVEVLGGERIETGYTPIDISLSLTQFLLSEFVPGAGFVLGLVDIIWGIFGPSQWDAFLVQIEQLINQRIEEFA
RNQAISRLEGLSNLYQIYAESFREWEADPTNPALREEMRIQFNDMNSALTTAIPLFAVQNYQVPLLSVYVQAANLHLSVLRDVSVFGQ
RWGFDAATINSRYNDLTRLIGNYTDYAVRWYNTGLERVWGPDSRDWVRYNQFRRELTLTVLDIVALFPNYDSRRYPIRTVSQLTREIY
TNPVLENFDGSFRGSAQGIERSIRSPHLMDILNSITIYTDAHRGYYYWSGHQIMASPVGFSGPEFTFPLYGTMGNAAPQQRIVAQLGQ
GVYRTLSSTLYRRPFNIGINNQQLSVLDGTEFAYGTSSNLPSAVYRKSGTVDSLDEIPPQNNNVPPRQGFSHRLSHVSMFRSGFSNS
SVSIIRAPMFSWIHRSAEFNNIIASDSITQIPAVKGNFLFNGSVISGPGFTGGDLVRLNSSGNNIQNRGYIEVPIHFPSTSTRYRVRVRYA
SVTPIHLNVNWGNSSIFSNTVPATATSLDNLQSSDFGYFESANAFTSSLGNIVGVRNFSGTAGVIIDRFEFIPVTA
SEQIDNO: 435
MEENNQNQCIPYNCLSNPEEVLLDGERISTGNSSIDISLSLVQFLVSNFVPGGGFLVGLIDFVWGIVGPSQWDAFLVQIEQLINERIAEF
ARNAAIANLEGLGNNFNIYVEAFKEWEEDPNNPETRTRVIDRFRILDGLLERDIPSFRISGFEVPLLSVYAQAANLHLAILRDSVIFGER
WGLTTINVNENYNRLIRHIDEYADHCANTYNRGLNNLPKSTYQDWITYNRLRRDLTLTVLDIAAFFPNYDNRRYPIQPVGQLTREVYTD
PLINFNPQLQSVAQLPTFNVMESSRIRNPHLFDILNNLTIFTDWFSVGRNFYWGGHRVISSLIGGGNITSPIYGREANQEPPRSFTFNG
PVFRTLSNPTLRLLQQPWPAPPFNLRGVEGVEFSTPTNSFTYRGRGTVDSLTELPPEDNSVPPREGYSHRLCHATFVQRSGTPFLTT
GVVFSWTDRSATLTNTIDPERINQIPLVKGFRVWGGTSVITGPGFTGGDILRRNTFGDFVSLQVNINSPITQRYRLRFRYASSRDARVI
VLTGAASTGVGGQVSVNMPLQKTMEIGENLTSRTFRYTDFSNPFSFRANPDIIGISEQPLFGAGSISSGELYIDKIEII
SEQIDNO: 436
MNPNNRSEHDTIKTTENNEVPTNHVQYPLAETPNPTLEDLNYKEFLRMTADNNTEALDSSTTKDVIQKGISVVGDLLGVVGFPFGGAL
VSFYTNFLNTIWPSEDPWKAFMEQVEALMDQKIADYAKNKALAELQGLQNNVEDYVSALSSWQKNPVSSRNPHSQGRIRELFSQAE
SHFRNSMPSFAISGYEVLFLTTYAQAANTHLFLLKDAQIYGEEWGYEKEDIAEFYKRQLKLTQEYTDHCVKWYNVGLDKLRGSSYES
WVNFNRYRREMTLTVLDLIALFPLYDVRLYPKEVKTELTRDVLTDPIVGVNNLRGYGTTFSNIENYIRKPHLFDYLHRIQFHTRFQPGYY
GNDSFNYWSGNYVSTRPSIGSNDIITSPFYGNKSSEPVQNLEFNGEKVYRAVANTNLAVWPSAVYSGVTKVEFSQYNDQTDEASTQ
TYDSKRNVGAVSWDSIDQLPPETTDEPLEKGYSHQLNYVMCFLMQGSRGTIPVLTWTHKSVDFFNMIDSKKITQLPLVKAYKLQSGA
SVVAGPRFTGGDIIQCTENGSAATIYVTPDVSYSQKYRARIHYASTSQITFTLSLDGAPFNQYYFDKTINKGDTLTYNSFNLASFSTPFE
LSGNNLQIGVTGLSAGDKVYIDKIEFIPVN
SEQIDNO: 437
ATGGATAACAACCCAAACATCAACGAGTGCATTCCATACAACTGCCTCTCTAACCCAGAGGTGGAGGTGCTCGGCGGCGAGAG
GATTGAGACCGGCTACACCCCAATTGATATCTCTCTCTCTCTCACCCAGTTCCTCCTCTCTGAGTTCGTGCCAGGCGCTGGCTT
CGTGCTCGGCCTCGTGGATATCATCTGGGGCATCTTCGGCCCATCTCAGTGGGATGCTTTCCTCGTGCAGATTGAGCAGCTCA
TCAACCAGAGGATTGAGGAGTTCGCTAGGAACCAGGCTATCTCTAGGCTTGAGGGCCTCTCTAACCTCTACCAGATTTACGCTG
AGTCTTTCAGGGAGTGGGAGGCTGATCCAACCAACCCAGCTCTCAGGGAGGAGATGAGGATTCAGTTCAACGATATGAACTCT
GCTCTCACCACCGCTATTCCACTCTTCGCTGTGCAGAACTACCAGGTGCCACTCCTCTCTGTGTACGTGCAGGCTGCTAACCTC
CACCTCTCTGTGCTCAGGGATGTGTCTGTGTTCGGCCAGAGGTGGGGCTTCGATGCTGCTACCATCAACTCTAGGTACAACGA
TCTCACCAGGCTCATTGGCAACTACACCGATTACGCTGTGAGGTGGTACAACACCGGCCTTGAGAGGGTGTGGGGCCCAGATT
CTAGGGATTGGGTGAGGTACAACCAGTTCAGGAGGGAACTCACCCTCACCGTGCTCGATATTGTGGCTCTCTTCCCAAACTAC
GATTCTAGGAGATACCCAATCAGGACCGTGTCTCAGCTCACCAGGGAGATTTACACCAACCCAGTGCTTGAGAACTTCGATGG
CTCTTTCAGGGGCTCTGCTCAGGGCATTGAGAGGTCTATCAGGTCTCCACACCTCATGGATATTCTCAACTCTATCACCATCTAC
ACCGATGCTCACAGGGGCTACTACTACTGGTCTGGCCACCAGATCATGGCTTCTCCAGTGGGCTTCTCTGGCCCAGAGTTCAC
CTTCCCACTCTACGGCACTATGGGCAACGCTGCTCCACAGCAGAGGATTGTGGCTCAGCTCGGCCAGGGCGTGTACAGGACC
CTCTCTTCTACCCTCTACAGGAGGCCATTCAACATTGGCATCAACAACCAGCAGCTCTCTGTGCTCGATGGCACCGAGTTCGCT
TACGGCACCTCTTCTAACCTCCCATCTGCTGTGTACAGGAAGTCTGGCACCGTGGATTCTCTCGATGAGATTCCACCACAGAAC
AACAACGTGCCACCAAGGCAGGGCTTCTCTCACAGGCTCTCTCACGTGTCTATGTTCAGGTCTGGCTTCTCTAACTCTTCTGTG
TCTATCATCAGGGCTCCAATGTTCTCTTGGATTCACAGGTCTGCTGAGTTCAACAACATCATTGCTTCTGATTCTATCACCCAGA
TTCCTGCTGTGAAGGGCAACTTCCTCTTCAACGGCTCTGTGATCTCTGGCCCAGGCTTCACCGGCGGCGATCTCGTGAGGCTC
AACTCTTCTGGCAACAACATTCAGAACAGGGGCTACATTGAGGTGCCAATTCACTTCCCATCTACCTCTACCAGGTACAGGGTG
AGGGTGAGGTACGCTTCTGTGACCCCAATTCACCTCAACGTGAACTGGGGCAACTCTTCTATCTTCTCTAACACCGTGCCAGCT
ACCGCTACCTCTCTCGATAACCTCCAGTCTTCTGATTTCGGCTACTTCGAGTCTGCTAACGCTTTCACCTCTTCTCTCGGCAACA
TTGTGGGCGTGAGGAACTTCTCTGGCACCGCTGGCGTGATCATTGATAGGTTCGAGTTCATTCCAGTGACCGCTTGA
SEQIDNO: 438
ATGGAGGAGAACAACCAGAACCAGTGCATTCCATACAACTGCCTCTCTAACCCAGAGGAGGTGCTCCTCGATGGCGAGAGGAT
CTCTACCGGCAACTCTTCTATTGATATCTCTCTCTCTCTCGTGCAGTTCCTCGTGTCTAACTTCGTGCCAGGCGGCGGCTTCCTC
GTGGGCCTCATTGATTTCGTGTGGGGCATTGTGGGCCCATCTCAGTGGGATGCTTTCCTCGTGCAGATTGAGCAGCTCATCAA
CGAGAGGATTGCTGAGTTCGCTAGGAACGCTGCTATTGCTAACCTTGAGGGCCTCGGCAACAACTTCAACATCTACGTGGAGG
CTTTCAAGGAGTGGGAGGAAGATCCAAACAACCCAGAGACCAGGACCAGGGTGATTGATAGGTTCAGGATTCTCGATGGCCTC
CTTGAGAGGGATATTCCATCTTTCAGGATCTCTGGCTTCGAGGTGCCACTCCTCTCTGTGTACGCTCAGGCTGCTAACCTCCAC
CTCGCTATTCTCAGGGATTCTGTGATCTTCGGCGAGAGGTGGGGCCTCACCACCATCAACGTGAACGAGAACTACAACAGGCT
CATCAGGCACATTGATGAGTACGCTGATCACTGCGCTAACACCTACAACAGGGGCCTCAACAACCTCCCAAAGTCTACCTACCA
GGATTGGATCACCTACAACAGGCTCAGGAGGGATCTCACCCTCACCGTGCTCGATATTGCTGCTTTCTTCCCAAACTACGATAA
CAGGAGGTATCCAATTCAGCCAGTGGGCCAGCTCACCAGGGAGGTGTACACCGATCCACTCATCAACTTCAACCCACAGCTCC
AGTCTGTGGCTCAGCTCCCAACCTTCAACGTGATGGAGTCTTCTAGGATCAGGAACCCACACCTCTTCGATATTCTCAACAACC
TCACCATCTTCACCGATTGGTTCTCTGTGGGCAGGAACTTCTACTGGGGCGGCCACAGGGTGATCTCTTCTCTCATTGGCGGC
GGCAACATTACCTCTCCAATTTACGGCAGGGAGGCTAACCAGGAGCCACCAAGGTCTTTCACCTTCAACGGCCCAGTGTTCAG
GACCCTCTCTAACCCAACCCTCAGGCTCCTCCAGCAGCCTTGGCCAGCTCCACCATTCAACCTCAGGGGCGTGGAGGGCGTG
GAGTTCTCTACCCCAACCAACTCTTTCACCTACAGGGGCAGGGGCACCGTGGATTCTCTCACCGAACTCCCACCAGAGGATAA
CTCTGTGCCACCAAGGGAGGGCTACTCTCACAGGCTCTGCCACGCTACCTTCGTGCAGAGGTCTGGCACCCCATTCCTCACCA
CCGGCGTGGTGTTCTCTTGGACCGATAGGTCTGCTACCCTCACCAACACCATTGATCCAGAGAGGATCAACCAGATTCCACTC
GTGAAGGGCTTCAGGGTGTGGGGCGGCACCTCTGTGATTACCGGCCCAGGCTTCACCGGCGGCGATATTCTCAGGAGGAACA
CCTTCGGCGATTTCGTGTCTCTCCAGGTGAACATCAACTCTCCAATTACCCAGAGGTACAGGCTCAGGTTCAGGTACGCTTCTT
CTAGGGATGCTAGGGTGATTGTGCTCACCGGCGCTGCTTCTACCGGCGTGGGCGGCCAGGTGTCTGTGAACATGCCACTCCA
GAAGACTATGGAGATTGGCGAGAACCTCACCTCTAGGACCTTCAGGTACACCGATTTCTCTAACCCATTCTCTTTCAGGGCTAA
CCCAGATATCATTGGCATCTCTGAGCAGCCACTCTTCGGCGCTGGCTCTATTTCTTCTGGCGAACTCTACATTGATAAGATTGAG
ATCATTTGA
SEQIDNO: 439
ATGAACCCAAACAACAGGTCTGAGCACGATACCATCAAGACCACCGAGAACAACGAGGTGCCAACCAACCACGTGCAGTACCC
ACTCGCTGAGACCCCAAACCCAACCCTTGAGGATCTCAACTACAAGGAGTTCCTCAGGATGACCGCTGATAACAACACCGAGG
CTCTCGATTCTTCTACCACCAAGGATGTGATTCAGAAGGGCATTTCTGTGGTGGGCGATCTCCTCGGCGTGGTGGGCTTCCCAT
TCGGCGGCGCTCTCGTGTCTTTCTACACCAACTTCCTCAACACCATTTGGCCATCTGAAGATCCTTGGAAGGCTTTCATGGAGC
AGGTGGAGGCTCTCATGGATCAGAAGATTGCTGATTACGCTAAGAACAAGGCTCTCGCTGAACTCCAGGGCCTCCAGAACAAC
GTGGAGGATTACGTGTCTGCTCTCTCTTCTTGGCAGAAGAACCCAGTGTCTTCTAGGAACCCACACTCTCAGGGCAGGATTAGG
GAACTCTTCTCTCAGGCTGAGTCTCACTTCAGGAACTCTATGCCATCTTTCGCTATCTCTGGCTACGAGGTGCTCTTCCTCACCA
CCTACGCTCAGGCTGCTAACACCCACCTCTTCCTCCTCAAGGATGCTCAGATTTACGGCGAGGAGTGGGGCTACGAGAAGGAG
GATATTGCTGAGTTCTACAAGAGGCAGCTCAAGCTCACCCAGGAGTACACCGATCACTGCGTGAAGTGGTACAACGTGGGCCT
CGATAAGCTCAGGGGCTCTTCTTACGAGTCTTGGGTGAACTTCAACAGGTACAGGAGGGAGATGACCCTCACCGTGCTCGATC
TCATTGCTCTCTTCCCACTCTACGATGTGAGGCTCTACCCAAAGGAGGTGAAGACCGAACTCACCAGGGATGTGCTCACCGAT
CCAATTGTGGGCGTGAACAACCTCAGGGGCTACGGCACCACCTTCTCTAACATTGAGAACTACATCAGGAAGCCACACCTCTTC
GATTACCTCCACAGGATTCAGTTCCACACCAGGTTCCAGCCAGGCTACTACGGCAACGATTCTTTCAACTACTGGTCTGGCAAC
TACGTGTCTACCAGGCCATCTATTGGCTCTAACGATATCATTACCTCTCCATTCTACGGCAACAAGTCTTCTGAGCCAGTGCAGA
ACCTTGAGTTCAACGGCGAGAAGGTGTACAGGGCTGTGGCTAACACCAACCTCGCTGTGTGGCCATCTGCTGTGTACTCTGGC
GTGACCAAGGTGGAGTTCTCTCAGTACAACGATCAGACCGATGAGGCTTCTACCCAGACCTACGATTCTAAGAGGAACGTGGG
CGCTGTGTCTTGGGATTCTATTGATCAGCTCCCACCAGAGACCACCGATGAGCCACTTGAGAAGGGCTACTCTCACCAGCTCA
ACTACGTGATGTGCTTCCTCATGCAGGGCTCTAGGGGCACCATTCCAGTGCTCACCTGGACCCACAAGTCTGTGGATTTCTTCA
ACATGATTGATTCTAAGAAGATCACCCAGCTCCCACTCGTGAAGGCTTACAAGCTCCAGTCTGGCGCTTCTGTGGTGGCTGGCC
CAAGGTTCACCGGCGGCGATATCATTCAATGCACCGAGAACGGCTCTGCTGCTACCATTTACGTGACCCCAGATGTGTCTTACT
CTCAGAAGTACAGGGCTAGGATTCACTACGCTTCTACCTCTCAGATCACCTTCACCCTCTCTCTCGATGGCGCTCCATTCAACC
AGTACTACTTCGATAAGACCATCAACAAGGGCGATACCCTCACCTACAACTCTTTCAACCTCGCTTCTTTCTCTACCCCATTCGA
ACTCTCTGGCAACAACCTCCAGATTGGCGTGACCGGCCTCTCTGCTGGCGATAAGGTGTACATTGATAAGATTGAGTTCATTCC
AGTGAACTGA
SEQIDNO: 440
TGAGCAGAAACTTGGATATCTGGCAGCAGAAAAACAAGTAGTTGAGAACTAAGAAGAAGGAA
SEQIDNO: 441
GTCATTGAAAAATTTGCAAAGCATCTGTAGCCACCCCACTTTGTTTGTTG
SEQIDNO: 442
MDNNPNINECIPYNCLSNPEVEVLGGERIETGYTPIDISLSLTQFLLSEFVPGAGFVLGLVDIIWGIFGPSQWDAFLVQIEQLINQRIEEFA
RNQAISRLEGLSNLYQIYAESFREWEADPTNPALREEMRIQFNDMNSALTTAIPLFAVQNYQVPLLSVYVQAANLHLSVLRDVSVFGQ
RWGFDAATINSRYNDLTRLIGNYTDYAVRWYNTGLERVWGPDSRDWVRYNQFRRELTLTVLDIVALFPNYDSRRYPIRTVSQLTREIY
TNPVLENFDGSFRGSAQGIERSIRSPHLMDILNSITIYTDAHRGYYYWSGHQIMASPVGFSGPEFTFPLYGTMGNAAPQQRIVAQLGQ
GVYRTLSSTLYRRPFNIGINNQQLSVLDGTEFAYGTSSNLPSAVYRKSGTVDSLDEIPPQNNNVPPRQGFSHRLSHVSMFRSGFSNS
SVSIIRAPMFSWIHRSAEFNNIIASDSITQIPAVKGNFLFNGSVISGPGFTGGDLVRLNSSGNNIQNRGYIEVPIHFPSTSTRYRVRVRYA
SVTPIHLNVNWGNSSIFSNTVPATATSLDNLQSSDFGYFESANAFTSSLGNIVGVRNFSGTAGVIIDRFEFIPVTAGSGAKQLLNFDLLK
LAGDVESNPGPMEENNQNQCIPYNCLSNPEEVLLDGERISTGNSSIDISLSLVQFLVSNFVPGGGFLVGLIDFVWGIVGPSQWDAFLV
QIEQLINERIAEFARNAAIANLEGLGNNFNIYVEAFKEWEEDPNNPETRTRVIDRFRILDGLLERDIPSFRISGFEVPLLSVYAQAANLHL
AILRDSVIFGERWGLTTINVNENYNRLIRHIDEYADHCANTYNRGLNNLPKSTYQDWITYNRLRRDLTLTVLDIAAFFPNYDNRRYPIQP
VGQLTREVYTDPLINFNPQLQSVAQLPTFNVMESSRIRNPHLFDILNNLTIFTDWFSVGRNFYWGGHRVISSLIGGGNITSPIYGREAN
QEPPRSFTFNGPVFRTLSNPTLRLLQQPWPAPPFNLRGVEGVEFSTPTNSFTYRGRGTVDSLTELPPEDNSVPPREGYSHRLCHAT
FVQRSGTPFLTTGVVFSWTDRSATLTNTIDPERINQIPLVKGFRVWGGTSVITGPGFTGGDILRRNTFGDFVSLQVNINSPITQRYRLR
FRYASSRDARVIVLTGAASTGVGGQVSVNMPLQKTMEIGENLTSRTFRYTDFSNPFSFRANPDIIGISEQPLFGAGSISSGELYIDKIEII
GSGVKQTLNFDLLKLAGDVESNPGPMNPNNRSEHDTIKTTENNEVPTNHVQYPLAETPNPTLEDLNYKEFLRMTADNNTEALDSSTT
KDVIQKGISVVGDLLGVVGFPFGGALVSFYTNFLNTIWPSEDPWKAFMEQVEALMDQKIADYAKNKALAELQGLQNNVEDYVSALSS
WQKNPVSSRNPHSQGRIRELFSQAESHFRNSMPSFAISGYEVLFLTTYAQAANTHLFLLKDAQIYGEEWGYEKEDIAEFYKRQLKLTQ
EYTDHCVKWYNVGLDKLRGSSYESWVNFNRYRREMTLTVLDLIALFPLYDVRLYPKEVKTELTRDVLTDPIVGVNNLRGYGTTFSNIE
NYIRKPHLFDYLHRIQFHTRFQPGYYGNDSFNYWSGNYVSTRPSIGSNDIITSPFYGNKSSEPVQNLEFNGEKVYRAVANTNLAVWPS
AVYSGVTKVEFSQYNDQTDEASTQTYDSKRNVGAVSWDSIDQLPPETTDEPLEKGYSHQLNYVMCFLMQGSRGTIPVLTWTHKSVD
FFNMIDSKKITQLPLVKAYKLQSGASVVAGPRFTGGDIIQCTENGSAATIYVTPDVSYSQKYRARIHYASTSQITFTLSLDGAPFNQYYF
DKTINKGDTLTYNSFNLASFSTPFELSGNNLQIGVTGLSAGDKVYIDKIEFIPVN
SEQIDNO: 443
ACTCGAGTGAGCAGAAACTTGGATATCTGGCAGCAGAAAAACAAGTAGTTGAGAACTAAGAAGAAGGAAATGGATAACAACCCA
AACATCAACGAGTGCATTCCATACAACTGCCTCTCTAACCCAGAGGTGGAGGTGCTCGGCGGCGAGAGGATTGAGACCGGCTA
CACCCCAATTGATATCTCTCTCTCTCTCACCCAGTTCCTCCTCTCTGAGTTCGTGCCAGGCGCTGGCTTCGTGCTCGGCCTCGT
GGATATCATCTGGGGCATCTTCGGCCCATCTCAGTGGGATGCTTTCCTCGTGCAGATTGAGCAGCTCATCAACCAGAGGATTGA
GGAGTTCGCTAGGAACCAGGCTATCTCTAGGCTTGAGGGCCTCTCTAACCTCTACCAGATTTACGCTGAGTCTTTCAGGGAGTG
GGAGGCTGATCCAACCAACCCAGCTCTCAGGGAGGAGATGAGGATTCAGTTCAACGATATGAACTCTGCTCTCACCACCGCTA
TTCCACTCTTCGCTGTGCAGAACTACCAGGTGCCACTCCTCTCTGTGTACGTGCAGGCTGCTAACCTCCACCTCTCTGTGCTCA
GGGATGTGTCTGTGTTCGGCCAGAGGTGGGGCTTCGATGCTGCTACCATCAACTCTAGGTACAACGATCTCACCAGGCTCATT
GGCAACTACACCGATTACGCTGTGAGGTGGTACAACACCGGCCTTGAGAGGGTGTGGGGCCCAGATTCTAGGGATTGGGTGA
GGTACAACCAGTTCAGGAGGGAACTCACCCTCACCGTGCTCGATATTGTGGCTCTCTTCCCAAACTACGATTCTAGGAGATACC
CAATCAGGACCGTGTCTCAGCTCACCAGGGAGATTTACACCAACCCAGTGCTTGAGAACTTCGATGGCTCTTTCAGGGGCTCT
GCTCAGGGCATTGAGAGGTCTATCAGGTCTCCACACCTCATGGATATTCTCAACTCTATCACCATCTACACCGATGCTCACAGG
GGCTACTACTACTGGTCTGGCCACCAGATCATGGCTTCTCCAGTGGGCTTCTCTGGCCCAGAGTTCACCTTCCCACTCTACGG
CACTATGGGCAACGCTGCTCCACAGCAGAGGATTGTGGCTCAGCTCGGCCAGGGCGTGTACAGGACCCTCTCTTCTACCCTCT
ACAGGAGGCCATTCAACATTGGCATCAACAACCAGCAGCTCTCTGTGCTCGATGGCACCGAGTTCGCTTACGGCACCTCTTCTA
ACCTCCCATCTGCTGTGTACAGGAAGTCTGGCACCGTGGATTCTCTCGATGAGATTCCACCACAGAACAACAACGTGCCACCAA
GGCAGGGCTTCTCTCACAGGCTCTCTCACGTGTCTATGTTCAGGTCTGGCTTCTCTAACTCTTCTGTGTCTATCATCAGGGCTC
CAATGTTCTCTTGGATTCACAGGTCTGCTGAGTTCAACAACATCATTGCTTCTGATTCTATCACCCAGATTCCTGCTGTGAAGGG
CAACTTCCTCTTCAACGGCTCTGTGATCTCTGGCCCAGGCTTCACCGGCGGCGATCTCGTGAGGCTCAACTCTTCTGGCAACA
ACATTCAGAACAGGGGCTACATTGAGGTGCCAATTCACTTCCCATCTACCTCTACCAGGTACAGGGTGAGGGTGAGGTACGCT
TCTGTGACCCCAATTCACCTCAACGTGAACTGGGGCAACTCTTCTATCTTCTCTAACACCGTGCCAGCTACCGCTACCTCTCTC
GATAACCTCCAGTCTTCTGATTTCGGCTACTTCGAGTCTGCTAACGCTTTCACCTCTTCTCTCGGCAACATTGTGGGCGTGAGG
AACTTCTCTGGCACCGCTGGCGTGATCATTGATAGGTTCGAGTTCATTCCAGTGACCGCTGGCTCTGGCGCTAAGCAGCTCCT
CAACTTCGATCTCCTCAAGCTCGCTGGCGATGTGGAGTCTAACCCAGGCCCAATGGAGGAGAACAACCAGAACCAGTGCATTC
CATACAACTGCCTCTCTAACCCAGAGGAGGTGCTCCTCGATGGCGAGAGGATCTCTACCGGCAACTCTTCTATTGATATCTCTC
TCTCTCTCGTGCAGTTCCTCGTGTCTAACTTCGTGCCAGGCGGCGGCTTCCTCGTGGGCCTCATTGATTTCGTGTGGGGCATT
GTGGGCCCATCTCAGTGGGATGCTTTCCTCGTGCAGATTGAGCAGCTCATCAACGAGAGGATTGCTGAGTTCGCTAGGAACGC
TGCTATTGCTAACCTTGAGGGCCTCGGCAACAACTTCAACATCTACGTGGAGGCTTTCAAGGAGTGGGAGGAAGATCCAAACA
ACCCAGAGACCAGGACCAGGGTGATTGATAGGTTCAGGATTCTCGATGGCCTCCTTGAGAGGGATATTCCATCTTTCAGGATCT
CTGGCTTCGAGGTGCCACTCCTCTCTGTGTACGCTCAGGCTGCTAACCTCCACCTCGCTATTCTCAGGGATTCTGTGATCTTCG
GCGAGAGGTGGGGCCTCACCACCATCAACGTGAACGAGAACTACAACAGGCTCATCAGGCACATTGATGAGTACGCTGATCAC
TGCGCTAACACCTACAACAGGGGCCTCAACAACCTCCCAAAGTCTACCTACCAGGATTGGATCACCTACAACAGGCTCAGGAG
GGATCTCACCCTCACCGTGCTCGATATTGCTGCTTTCTTCCCAAACTACGATAACAGGAGGTATCCAATTCAGCCAGTGGGCCA
GCTCACCAGGGAGGTGTACACCGATCCACTCATCAACTTCAACCCACAGCTCCAGTCTGTGGCTCAGCTCCCAACCTTCAACG
TGATGGAGTCTTCTAGGATCAGGAACCCACACCTCTTCGATATTCTCAACAACCTCACCATCTTCACCGATTGGTTCTCTGTGGG
CAGGAACTTCTACTGGGGCGGCCACAGGGTGATCTCTTCTCTCATTGGCGGCGGCAACATTACCTCTCCAATTTACGGCAGGG
AGGCTAACCAGGAGCCACCAAGGTCTTTCACCTTCAACGGCCCAGTGTTCAGGACCCTCTCTAACCCAACCCTCAGGCTCCTC
CAGCAGCCTTGGCCAGCTCCACCATTCAACCTCAGGGGCGTGGAGGGCGTGGAGTTCTCTACCCCAACCAACTCTTTCACCTA
CAGGGGCAGGGGCACCGTGGATTCTCTCACCGAACTCCCACCAGAGGATAACTCTGTGCCACCAAGGGAGGGCTACTCTCAC
AGGCTCTGCCACGCTACCTTCGTGCAGAGGTCTGGCACCCCATTCCTCACCACCGGCGTGGTGTTCTCTTGGACCGATAGGTC
TGCTACCCTCACCAACACCATTGATCCAGAGAGGATCAACCAGATTCCACTCGTGAAGGGCTTCAGGGTGTGGGGCGGCACCT
CTGTGATTACCGGCCCAGGCTTCACCGGCGGCGATATTCTCAGGAGGAACACCTTCGGCGATTTCGTGTCTCTCCAGGTGAAC
ATCAACTCTCCAATTACCCAGAGGTACAGGCTCAGGTTCAGGTACGCTTCTTCTAGGGATGCTAGGGTGATTGTGCTCACCGGC
GCTGCTTCTACCGGCGTGGGCGGCCAGGTGTCTGTGAACATGCCACTCCAGAAGACTATGGAGATTGGCGAGAACCTCACCT
CTAGGACCTTCAGGTACACCGATTTCTCTAACCCATTCTCTTTCAGGGCTAACCCAGATATCATTGGCATCTCTGAGCAGCCACT
CTTCGGCGCTGGCTCTATTTCTTCTGGCGAACTCTACATTGATAAGATTGAGATCATTGGCTCTGGCGTGAAGCAGACCCTCAA
CTTCGATCTCCTCAAGCTCGCTGGCGATGTGGAGTCTAACCCAGGCCCAATGAACCCAAACAACAGGTCTGAGCACGATACCA
TCAAGACCACCGAGAACAACGAGGTGCCAACCAACCACGTGCAGTACCCACTCGCTGAGACCCCAAACCCAACCCTTGAGGAT
CTCAACTACAAGGAGTTCCTCAGGATGACCGCTGATAACAACACCGAGGCTCTCGATTCTTCTACCACCAAGGATGTGATTCAG
AAGGGCATTTCTGTGGTGGGCGATCTCCTCGGCGTGGTGGGCTTCCCATTCGGCGGCGCTCTCGTGTCTTTCTACACCAACTT
CCTCAACACCATTTGGCCATCTGAAGATCCTTGGAAGGCTTTCATGGAGCAGGTGGAGGCTCTCATGGATCAGAAGATTGCTGA
TTACGCTAAGAACAAGGCTCTCGCTGAACTCCAGGGCCTCCAGAACAACGTGGAGGATTACGTGTCTGCTCTCTCTTCTTGGCA
GAAGAACCCAGTGTCTTCTAGGAACCCACACTCTCAGGGCAGGATTAGGGAACTCTTCTCTCAGGCTGAGTCTCACTTCAGGAA
CTCTATGCCATCTTTCGCTATCTCTGGCTACGAGGTGCTCTTCCTCACCACCTACGCTCAGGCTGCTAACACCCACCTCTTCCT
CCTCAAGGATGCTCAGATTTACGGCGAGGAGTGGGGCTACGAGAAGGAGGATATTGCTGAGTTCTACAAGAGGCAGCTCAAGC
TCACCCAGGAGTACACCGATCACTGCGTGAAGTGGTACAACGTGGGCCTCGATAAGCTCAGGGGCTCTTCTTACGAGTCTTGG
GTGAACTTCAACAGGTACAGGAGGGAGATGACCCTCACCGTGCTCGATCTCATTGCTCTCTTCCCACTCTACGATGTGAGGCTC
TACCCAAAGGAGGTGAAGACCGAACTCACCAGGGATGTGCTCACCGATCCAATTGTGGGCGTGAACAACCTCAGGGGCTACG
GCACCACCTTCTCTAACATTGAGAACTACATCAGGAAGCCACACCTCTTCGATTACCTCCACAGGATTCAGTTCCACACCAGGT
TCCAGCCAGGCTACTACGGCAACGATTCTTTCAACTACTGGTCTGGCAACTACGTGTCTACCAGGCCATCTATTGGCTCTAACG
ATATCATTACCTCTCCATTCTACGGCAACAAGTCTTCTGAGCCAGTGCAGAACCTTGAGTTCAACGGCGAGAAGGTGTACAGGG
CTGTGGCTAACACCAACCTCGCTGTGTGGCCATCTGCTGTGTACTCTGGCGTGACCAAGGTGGAGTTCTCTCAGTACAACGAT
CAGACCGATGAGGCTTCTACCCAGACCTACGATTCTAAGAGGAACGTGGGCGCTGTGTCTTGGGATTCTATTGATCAGCTCCC
ACCAGAGACCACCGATGAGCCACTTGAGAAGGGCTACTCTCACCAGCTCAACTACGTGATGTGCTTCCTCATGCAGGGCTCTA
GGGGCACCATTCCAGTGCTCACCTGGACCCACAAGTCTGTGGATTTCTTCAACATGATTGATTCTAAGAAGATCACCCAGCTCC
CACTCGTGAAGGCTTACAAGCTCCAGTCTGGCGCTTCTGTGGTGGCTGGCCCAAGGTTCACCGGCGGCGATATCATTCAATGC
ACCGAGAACGGCTCTGCTGCTACCATTTACGTGACCCCAGATGTGTCTTACTCTCAGAAGTACAGGGCTAGGATTCACTACGCT
TCTACCTCTCAGATCACCTTCACCCTCTCTCTCGATGGCGCTCCATTCAACCAGTACTACTTCGATAAGACCATCAACAAGGGC
GATACCCTCACCTACAACTCTTTCAACCTCGCTTCTTTCTCTACCCCATTCGAACTCTCTGGCAACAACCTCCAGATTGGCGTGA
CCGGCCTCTCTGCTGGCGATAAGGTGTACATTGATAAGATTGAGTTCATTCCAGTGAACTGAGTCATTGAAAAATTTGCAAAGCA
TCTGTAGCCACCCCACTTTGTTTGTTGTCTAGAA
SEQIDNO: 444
TACAAGCCGCACAATACAAGTCGCCTTCTCAAACCCTTCCTCACGACATTCTCGGACCTTATGACACCGTCACCAAAACAATGAT
CCACGCGATATTAGGCGCGTGCAAATCACTCTAATCCGAAACTAGTAGACATGGGAAGCACGAGCTATACGCGAGCGTTTCAAT
TGCCGCCACGAAAGCAGAGAAGGCCAGAAACGGAACCACGGTAAAATGGTAAGGGTATTTTCGTAAACAGAAGAAAAGAGTTG
TAGCTATAAATAAACCCTCTAACCCACGGCGCACTATTTCTCTTCACTCCTTCGTTCACTCTTCTTCTCTTGCGGCTAGGGTTTTA
GCGCAGCTTCTTCTAGGTTCGTTCTCTTCCGCCGCTCTATGGATTTTAAACCTTCGAATCATGTTTATTCCATTGAATTATGTTGC
TTGCAGTTTATATTTTCTGAATCTGTAGTTGTTGTCTTCAATTTATCCTATGCTTTATAGATCAATCTTTTGTGTGTGTAGTACGTA
ATTTTTGTTCTTTTTGCTTTTCGTTCAAGTTGTTGGGAATAATCGGGGTATCATGTTTTGATATTGTTTGTTTTCTTTTTTGACTGCT
TAATAATTTTTAAGTTGGTTTTGGTTTTGGGGTTTTATGTGCTTGTTATATTCAAATCTTTGGATCCAGATCTTACAAAAGTTTTGG
GTTTAAGGATGTTTTTGGCTGATGATGAATAGATCTATAAACTGTTCCTTTTAATCGATTCAAGCTTAGGATTTTACTAGGCTTTTG
CGAATAAATACGTGACAGTAAGCTAATTATGTCCTTTTTTTGTCTCAATCATATCTGTCTGGGTGTGCCATAATTTGTGATATGTC
TATCTGGTAGAATCTTGTGTTTTATGCTTTACGATTTGGTATACCTGTTTTTGAACTTGTTGTATGATGGGTATTTAGATCACCCTA
TCTTTTTTATGCTTCTGGAAGTTTTATGTAAATGTCGAATATCTTAATGTTGTTGAACTTATAATGTTGTGTTGATGTATGTATGAT
GGTTTTGACAACTTTTTTCACTGGTTCTGAAAGTTTTATGTAAATTGCAAATATGTTAATGTTGTTGAACTTATTTTTTTTCCTTCGA
TGTTGTTTTGATGTATGTATGATGGTTTTCACCGTAGTTTCTATGGCTAATATCTTAATGTTGTTGAGCTTATTTTTTTCCTTATATG
TTGTGTTGATGTATGTATGATGGTTTTGACAACTTTTTTAGTTTCTTTCAGATTTAAGGCTCTAGA
SEQIDNO: 445
TGAGTCATCAAATGCTTGGGCTATCAGGGGGTTGAGCTCCACAGTGCTTTGAGCTCTTAGGAATGTCATTACTATTCCACTTACC
TACCTTTGGTGGAGTTTCATTAGGGATGATGCTGCATTTCCAACATCTAATCGTTAAAGACTTCCAAGAAATCTAAGTAGCTTTTC
TTTATTTAGAAGTCCTTATTTCTTTTTATTCTTATAACAATTTAGATGCCTATCTAGTTTTGTATTCATAGATGGGAAAATGATAAAG
TTTACCTTATGTAATATCTTCTTTAATAATGTATCAACATCATTTTTCCTAAGTCTTTGACTAAGTGATTGACAATAATTATAAAAGA
TAATTAAATTTATTTTTATAAAAAATTTAATTAATAAATATATGTGAATTTTTTGTTTAAATTTCAATAGACAATTATGTTGAAAATGA
GATTTCGTTACACACTTTGACAATTATGTTAAATTTCTATTTTCTTTCTGGTTGGCTTTGTTGGGCTTCACACTCCAGTTTGGCAGT
TTAGGAAGAGAGCAAGAAGACCTGACTCAGCCCAAAAGTGCACACCATTGAAAAAAGATCATGTCAATTATATTTTTTCAAAGTT
TAAAATTATGATAAAAAGAAAGAAAAATATTTTCGATTCTTATCAATTATATTTTTTTTTATAAGGATTCATGTTATTTATATTAATAT
ATATCATTTATGTAATTAAAAAATCATATATTTTGTGTAAAATAAATCGTATTGATTTACTGCCTCAATTTAACATTTAATTTAAAGA
GACTTTCTATCATATTTTTTTGAAAAATCTTTCTATCTTATCCTAATAACTCCAAATAAATAATAAATCATTATCATCATGATAATAT
GAAAGAAAGAAAAACATCGGGCCAAGAAGTCAATCAGGCGTGAGAGATCTAGGCCCAAGGCCCATATACTTTAGAATTTCGTTT
GCATTGCGTGGTGGATACAACCCTAAAAAAAACACTACACTTCTCTCTTCGTGTCTCTCACACACACCACTTTATATACCCACGC
AATAGAAGGGAGTGCTACTCTCTTCGCCGCTCTCTCTCTCCTTCTCGCTCTTTCAGGTTCGGTTCCTCTCTGCCTCTAATTTTTC
ACTCGCTTTTCCTTTGCTTCTTTGTTTCCGAAACGATCGGTACCTTTGGTTTTTCTCTCTTCCGTTTATTATCTTCAGATCTGTGCT
TTACTCAAATGTTCAGATTCCGTTTTGTTGTTTTCCTGCGTGCTTTTTAATAAGCTTCTTTCTTGTTTAATACTTTTCGGTGAATCG
GTGTAGTTTTGTTGATTTGCTTGTTGTCTATGTATGGGGTTTTTGCTTTCGCTTACTTTTATACGGAATAAGAATAATCGATTGTTC
TGCTTTTCTTTTTTGTTGTTGTTTAGTAAGCTTTGATATGAATTGATTGAACATGATATTAGGTGATCCAAATCTAAACAGACTTTT
TTTAAATTTTTTTATTTCGTCTTTGTTCGGTTGTTTTACTTTCTGTTTGTGAGTGATGGATCTTGGTTCAACACACACCGACGCACT
TGACTGTTGAGATGTTTTAGCTCTAGACGTGCGCTATTGTTTTTTGATAATTTGTTGAATATTATTTGCGTTCCCTAAAATGATAAT
TTAGGGTTTTAAGCAAATCGCATTATGTGATGAGCCCGTTTGCTGCTTTTGTTTGAAATTTAAAGTTTGTTTTTTTTAAACAATCCA
AACTTATTTCCTGAGGGAGAATAAGGAATTATAACTGTTCCAAGAGAGAAAATTTTAGGTGTATATTAAGATGAGATTTTTTATCT
GTTATGGTCATATTTGTGGTTTTTTTTTACTTATTTGTTTTTTTCAATTTGTTTATGACCGAGTTATGACAACAAATCTTTTGCATCC
TTTTTGCTCTCTTTAAATATCTCTTTTTTCTTTTTCTAGTTTTTGTTGCCTGCATTAAGTATCTTGCTATTTAGCTAACGTTGGTGTT
TTAATTTTTCAGCTTTGACTTTTGTTGGACTAGA
SEQIDNO: 446
CTAGTTAATTACACAAATTTTAATTCATCTAGTAAATTTATGTGTTTGTGTGTAAGTTATTTCAATTAGCTTAAGCTATTTTTTTCATT
TTTATCTTACTAATTTACTTGAAATCTATTTTTATCTTTTTTATTTAGAAACTCTTTTCGTCTACCATGCCACTCTCAATGATATCAC
GTCTTTCACTTCTTCCACTTTCACATTCACACAACGTCAGCATGGTTTAATACAATTGATAAATAGTTAGATATCCTAAACATGTGT
TCTATGAATTTGATTCTTGACTATATGCATGAAAAAGATTATTAAAATAAATAAATAACCAAAGTCACTCTAATTATAAATTTTCAGC
TATCTTTTAAGAAAAAAAAATCTCAAGCACATTCTAAATAAATTTTTAACTAATTTACAGCTATCTATACACATTCACAATGAAGGA
ATGTGAGAATTGTTAGAATATTTAAGAATATGAAAATATTAAAAAAAAAAAACTAAGAAGCCAGAGTTTCCACTTTCCAATATGCA
CTTAAATAAATAGTTCAATTAAGGGTATGTTTGATATAAATTAAATTTTATTTGTAACTTTAGCAATAAAATAAAACTGATTAAAACA
TGTTAAAAAAATTAATAAGTGTTATAAAATATTTTTTTTAAATGAATATTTCCATAAATTTAAATAATAGTATCTTTCAAATATTGACC
CAATAGAAACTAGCCAGCCTATAAATAAATAAATAAATAAATATGAATTAATGTTGATTTAATAAGGGAAGAAAAAAAGGTAGTAG
AAGGGTGTGAAATGAAAAAGAAAAGGCTGTCATCCAGGTCACCCTAACCCTCTTTTCTCGTGGTCGAAATGATTCCTTCTATTTC
AATTTTTGGTCCCAACCCCAATTTTAATTTCAAATGCATTACATTACACTCTCTCACTCTCACACCAACCACACACTCTCTCTCTCT
CTCCGCATATATATACCCCTCTTCTTCATTCTCCTCTTTCTCTGTTCTCATTTCTCTTTCTGTAGCCCTCAATCACCGGAGAGAGA
GTGAGTGCAAAGCCATCGTCTTCTTCTCAGACAAGGTATTCATCTTAATCGCTTGCATCATAGCTTTGATGCAAAGTACGTTTCC
TTTTGCTCTTTGATTCAGCACTTTTTTTTTTTTTTTTTGCAATCTCGCATGTTTATTCTTAGATTTGTTCAACAGTACTACTCTTTATT
TTTTAATCTGTGTGACATTGCTACCTACCTCATCAAAATCATATCTGGATGTTTTTGTATCTGATTATTTATATTCGTACTGCCTGA
TCTATACTGAATGAGATGTAGTGGATTGTGGCGATTTGTAGTTTTAGTGATGCGGATGGCATATCATAGTTCCGATCTTCTAGAC
CTGAGATCTTCGATCTTTTTAAACTGTACTTTTACTTATTTATTTTAAAATGGGGATAGACATCGAATCTATAATCTTGTGTTGGAT
GGCTTTATGGTCTGATCTGTTTTTTATGTGGGAATCTCTCACTTTTTCTTTTTTATTTATCATTGATCTTTCTTTGTTGAAGATCATC
TAGTGTTTGGGAACTTAAGATATCCTGATGAATGTTGAGATTTTAGTAGATATCCTAGTAAGTTTGAGATTACTAGCTGCACATAC
TCCCTTCCTATGTTTATTTTATTCGCCATTTTCTGGACGTTTTCCATTCATTTATAAATTTTGGCAGTTTTACTTTAAAAAAATGATC
AGGATCGGCTCCTTTGTTTGTCAATTTATGTACCTTTTCTTTCTAAGCAATCATAAAGTAGATTATATAATACTGAATTATTTCATTT
GCTTGCTTTCTTCCCATTGATTTTTGTGTGTTATATACAATGTACTGTTGCTGTCTCATATAGAAAATAAACGTAATCTTCAAAGTC
TGTAAGTGGCTGGGTTCTGCAGATTGTCTATTCAAACTTGTTAATACTGGCAATTTGTTCTATGCATACAGTAGTTTGTTAAAAGA
CTAGA
SEQIDNO: 447
ATCTTTAATTGGTTGTTGCTGTCTCTCATTCCAAACAAGTGTGTTCTGTTCTGTCTTTACTGTATGTTGTTCCATGTATGTGTTTAT
GCTGTGTGTTGTGCTTTATGTCATTTGTGATGGTTTAATACTGGGGAAGTTGAAAGTTTTCCGTTAAATGTATCAATAAACTTTTA
TTGCTTTATCTCGATATGATTTTCTAAATCTCTCAGTCGAC
SEQIDNO: 448
ACAATTTAACCCTTCTTTTCTGGGTTGCAGGATAAGACATTGGTTCTTTTGAATTTGCGTGGTGTTTTAGTTTAGGTTTGTTATGT
ATCATACGAATCTCTGGTTTGCATAGAAAATATTTCATTTGAAATTTAAATGGAGTATCGTTTCTGTTAGGCATATTATTTCGGCTA
AATTAGTTTTTTTTTCTTCTATTCTATTTTATTATTTAGGTGTAATTTGTTTTTTTTTTATGTTTTGAGATTCAAATGATGATTTAAAAT
TATTTAAAGATAAATTTGAATTGTCACAATAGTCGAG
SEQIDNO: 449
ACTTGTTGTCGCTGAAAATCCTGAGCTTTAAAATTTGCTTTGTTGTAGTGTATTAATTCTCTCGGTCTTGTGTTCCAATGATGATTT
TGAGAAAGATCATGGAATTGCGAATTGTACCTGCTTAAGTAAGTGGTAACTGTGTGGTTGTGTAATTCGTGTGAATAAACACATG
CTTTCAGAAAATTTGCTGCATGAGTCAGGTGTCTGATACATATGTGAATGTGATGTAGCCAAGAAATAGACCGTGTAATTATTGG
CTAATTGCTTTTGTTATAGCTGAGACACACTATGTGAATTAACTGCAGCTGGCCAGTAATTGAATTTGATTATATTGTTTGTCGAG
SEQIDNO: 450
ACCGTGCAGGGTGACCTAGCTAGGGGAGTTTATTTTCATCATTCCAATAATAAATGGTTCTTGATCCCTAGTATTTCTATTTCTGG
TCTAGTTTTGTTTTTTGGATTAAGTGTGAAGTCTGCGCCGTCATCTCGCAGCTTTTGTTCCCAGAACTGGGTTCTAGATCGACGG
TGGCAGGCTTTTTTTGTTTTTCTTTTCAAGTTATGTTTAAGCAATTGTTTGTCATGGGTGAAGTGAACACATGAATTTTATAAGCAG
CTTTACTCTGCTTATTTGGTTATCGAATATATCAACTATATGATATTTCTGTTTTTTATTCTATGGTGCTTCTAAGTTGTTGGGTTG
SEQIDNO: 451
TTTCATCATGTTGGTTACAGAAAAAACTTTCATAGTAGTTTATCCTTACAGAGTCTTAGGCCAAAGTCATGTATTCTTATACAAGG
TTCATGGTCGCCGGACAATTTTTTCTTTGTTGTCTGTTCATGAGAGTACTATTTTTGTTTTCTTTTATGTAAGAGTCTGAGTTTTCT
ATTTAAGAAGCAGTAAGAGGGTTTAGCCCTTTTTCTGCCGAGAGATGTTTTAACTGCGTTTTTTTTTATATGAAAGTTTTGTCAAT
GGATTATCGTTTTTATACATTTTTTACATTTAGGTAAAAGTAACGTGAGCCGGAT
SEQIDNO: 452
ACCATCCCTATGTTTGACACGAACTTTTCATTAGGATAATCAAGTAAGCTTTTTTGGAAAGTATCATATTTCATGATAGAGGTTCG
TTAATTCTCGATAAGAATAATAATAATAATATTTTCGAAATTGAAACTTGATTATAAAATTATAAGAAAATAAAATCTCAATTGCAAA
ATATATAATTAAGATTAAATGAAAAAAATATCTTAACATGTCATTTATTTTCTACATATTTATTTATCTCACTCATTATAACTCTTAGT
ATTTTAAAATTTATAATAAAAAATAAAACATGTAATTTTAAAATATAATCAGTGATAATATTTGTGAAATTAAAATCTGTGCAAAAAT
AGATAAGATGTGCGGCTAAGCCAAGACATTAATCATCTGAAAAGACTTAATAACGAAAGATTTTATTCAAATGAACGAATGCGAA
TGTTGAAACGGTGCCGTTCATTTTCTTAACCTAGTCACCTTGCTGAAACGGTGTCGTCATAGTAGCGCAGCTTTCCTGTTTAGTC
CTTGCCAGCTGGACTATGTCGAACCCACAAACCCCGACATGACATAAACACAAAGCGCGTGAAAATCAACCGTACAAGACCATC
GAGAAACCACACGCGTCAATCCCACGCGTCATTCCACGCTAGTGGCACATAATAACGTTCAAATAAGCAAACCGGGGCATAATT
GGAAACCTGATTAAACCCTAAGGTCATTATTGGAAACGAGAAGAAGCTCCCACAATATAAAAATCTTGGAACCCTAGTTTTATTA
TATTCGTGCTTCCTTACTCTGTTTCTCAGTCTGTGTCTTTGCGGCTGAGGATTCCGAACGATTGGCCTTCGCTTCTCGCTAAGGT
AGTAGCCTCTGCTCTCTTGTCTCGCTGATTTCGTTCATCTCTGTTTCTTGTTTACGATGATGATTCTTCGTATTTGTTTTTTAATTT
TAAATTTCCCGTTCTGTGATTTGTTTTACGTGGATTATCTACATTTTCTACCGATTAACGGAAGAGGTTCTCTTTTCGGTGTOGAA
CCTCGTTGTGTTCACAGATATATTGCTTTTGTGTCATAATTATGCGATTTTGTTTGTTCTTCTTTTGAGATTAGATGTTTTCAATGA
TTTATTGTGGTTTATGACTGGTTTATGTTTTCATGCAACAGTGTCTATATTTTTAATGACTTTTTTTTTATGGTGTCTGGAGTTTTCA
CCCACGTTTCAAGTTTGCCATGCTTTGATACAAAGCTTGCATAATATTTTAATTTAATTTCTATGCGTATATGTTTGAATCCTGCTG
ATGTTTTTATTGTTGCCCATTTTGTGAATTTTTTGTAACATGAAATTAATTTTTTTATGAAGTTGTTTTTGTTACTGCAAAAAAGAAA
CCTTTCGGGTATGATAAATCTGCCGGGAGTGTTTTTCGTTATGTTCTGTACGATTGTGCTTTGATTAAATGTATTTTTAATTTATTT
TCTTATATTTTGCTGGAGATGTCGAGTTGAAACTTTTTCTTAATGATTTGATTATTTTTCTGCTTGATTGAAAAGATTTTTCTACGCT
ATGATGAATCTGCCTTTTTAGATGCAGGCTGGTTCATATTTTGGAATAGAACTTTTTTGGCTTAACAATTTTTTGCAACTCTTCTTT
TACCTAATACATGTGTGAAGTTGGATTTACCTTGTCATTTATTTTGCCTGATAATTCACTTTCTAGCGATTGTGGCTTCTGATTTGT
GTTTCTTTGAATTGGTTTCAGTTTAACTCAAC
SEQIDNO: 453
agcttttggagagtttcatatctcatgatagaggttcgttaattttcgataagaatattaataataatatttccgaaattgaaacttgattataaaattataagaaaataaatctcaattgcaaaatat
ataattaagattaaatgaaacaaatcttttaacatgtactatttatttgtacatatttatttatctcactcattaaaacgcttaatattttaaattttataatagaaaataaaacatacgtaattttaaaatat
aatcagtgataatatttgtgaaattaaaatctatgcaaaaatagataagatgtgctgctaagccaaaacattaataatctgaaaagactatttagattttattcaaattaactaatgcaaatgttga
aacggtgtcgttgattttcttaacctagtcacctagctgaaacggtgtcgtcatagtagcgcagttttcctgtttagtccttgccagctggactgtgtcgaacccgcaaaccccgacatgacgtaa
acacaaagcgcgtgaaaatcaagtgtacaagactcccgagaaaccacacacgtcagtaccacgcgtcattccacgcgagtggcacacaataagttcaagtaagcaaaccggggcat
aatcggaaagctgattaaaccctaaggtcattattggaacgaaaagtagctcccacaatataaaaatcttggaaccctagattttattatcttcgtgcttccttgctccgtttctcagtctgtgtctttg
cggctgaggattccgaacaattggccttcgcttctcgctaaggtagcagcctctgccttcttgtttcgttgattttgttcatctcttacgatgatgattcttcttattttttttttccaattttaaatttctcattctg
tgttttgttttatgtggattatttatgctttctaccgattaacggaagaggttctcgtttcgatgttgaacctcgttgtgttcacagatctattgcttttgtgttataattatgcgattttgtttgttcttcctttgagat
aagatgttttgaatgatttattgtggatctgttattactgaactattatgaatggttttggttttcatgcaacagtgtctatgtttttaatgatttttttttgttgtctggagtttttgccccatgtttcaagtttgccat
gttttgatacaaagctggcataattttttaatttaatttctatgcgtaaatgtctggatcctactgatgtttttattgttgcccattttgtgaattttttgtaacatgaaattatatttttgtgaagttgttttttaatcc
tgcacaatacaaacctttcaggtacgatacgtctgcgggagtgtttttggttatattttgtacgattgtgatttgattaattgtatttttaatttattttttttatttgctggagatgtcgagttgaaactttttctt
aatgatttcattatttttctgcttgattgaaaagatttttctacgctgtgatgaatctgccattttagatgcaggctgattaatattttggaatagaacttttttggcttaacaattttgcaattcttctcttacct
aatacatgtgtgaagttggatttaccttgtcatttattttgcgtgataattcactttgtagcgattgtgggttctgacgtgtttctttgaattggtttcagtttaactcaa
SEQIDNO: 454
TGACAAGGTCAAGAAGGAGTTGCAATGTAACCTCCACATCTTGAATGTTAATTAAAGTTGTAAATTTCATGAAGAGGTTAGTGTT
CGTGGTATTATTAAGGATGTGAAACAAATTAGTATAAATAGTATCATCAAATAATAAATTAAACACCATATCATATTAATAAAGAAT
AAATAAAAAAATAAATAAGTATAAATATATATACATCGTTCGTTTTAATTTGTATCAATTTTAAAAAATCAATCTAAAATCTAATTTGA
ACAAAATAATTTTTATAAAATGACATTCAAACGCATTCTATAATATTCAGTCTTTTTAATACTCCCTCCGTTCTTTTATAAGTGTCAC
TTTCTTGCCAAAAATTTGTTTCTTTTTAATTGTCACTTGCAAAGTTCAAGGTAGTATTAATTGCAATTTTGTCAAAATTATCCTTAGA
TAATGATTGCAGAGAGAGAAAAAATAAAGTGAATGTAATAAATAATTAAGGGTATTATAGGTAAAAGAAGAATTATTGTTTGAAAA
ATAACAATAATAATTAGCTTCCTTGATATGCGTAAAAAATCAAAAAATAACACTTAAAAAGAAACGAAGAGAATAATATTTTAAAAT
CAATTTATTATTTTTATTTAGATCGGTTTGAATTTAAACATCAATAATATAATAAATTAAATAAATAAAATTTACATGTACAATTTATA
AAAAAAAACTCGCGTTATTAAATATTGTTTTAATATAAATTTATTAAATGACTTTATTAGGTCAAATCACTCGGCTAAGATTAGACA
GAAACCTTAAAATGCTCCCTCTACAATTGTTATGCCGCGGCGGAACGAAATAAAAGAAAGGGCAATTCCGTCAAAATAAAAGAA
ACAATTGTACTATATAAATCCCGTAACCCTTGTTTTGTCTCACTACACTATTGTATTTTATTCTCATTCTATTCCTCCTAGGGTTTTA
GCGCAGCTTTCACACTCAAAGGTAAATCTCTCTTTTTTTCGTTTATCATCGATTCTTATTATCGATATCATCGATTCTCATTATCGA
TATCATCGATTCATATGTTTTCTTTTGCACAATTTAGTGTACATATTTGATTACATAGGATCATTAGAAATTGTTTTGTTTTTCTTAT
GGTTCCTGCTATTAGATTTCGATAATCATGATGTGATTTATGTGTCTGTTTAATGGTTAATTGTTATTCTGAATCCTGATAGTGTGA
TAATCGTATATGTTTTTTTAATCGATAGTTTTTCTTTCGTATGGTAGTATTTATTTTTGGGTATAGATATATTCAGTCGAGATGCTTG
ATGCATATAATATGATATGATACATCTGATATTTTTAAATTGTACATGTGTTTGTTTTTCGAGTATGTTGTGCCTTCTCGATTCAAA
CATGCTATGTTGAAATTAATTCTATGTTATTTTGATTTGTCATATTGCTTTTTGTTGTCTGTATTTGTTTATTTATTTTTGTGGTTGAA
TACTGAGGTTTATTTTACTTATGGATTGTTTTCTGTGATGAAATTGTAGATCTTTTGTGATTTCAAGCTCAAGGACTA
SEQIDNO: 455
ACATGGTACACACCTCTGTCACCATAACTCCCGCCATCTTCTTTATGAAGATGGTTGACAAGACTCTATATTCTAACACAAACAC
CATTGTCAGAGGTTTCGTTGGTGGAGATAACTCTAGCTTCCCGAAGAAAACACACAAAGCAGGTGCTTGCAATAAATGTAATATC
TCTTGGTTTCCCAAGCGGCGCTTGAGGGAAAATAGGGGGTTATCATCACCTTTTCCAAGGAGGACACCCTCATAGTCAATCCTC
ACTATAATGACCCGTTGGTCATCACTGTGCAACATGACAACTTGGTTATCAAACGAGTATTGATAGATCCTGGTAACTCAACTGA
TATCCTATTCTAGGACGCCTCTCAAAAGTTGCAGATCAATCATGACGTCAAAATGTTCAACGACTCTTTAACAGGATTGTCATGC
GAACAAGTATAAGTAATGACTCATATGACCCTGGAGACCACATGTGGCGAGGGTGCAAACGCTAACGCGATTACGGTCAGCTA
CTTTATTAAAAAATAATTTATCATTTAAAAATTAGACATATTAATTTTAAATACAAAAGTATAAGAGGAGATCAAAACTATTAAATTT
AAAATAGAAGAATTACTTTCGTTAATAAAATTAAATAATAGGCAAAACTGTAATTAAACTTAAATAAAAATTAAATAAATAAAAAATA
AGAAAAAAATGGAATAAATGATGATTATAAAAGATTACTATTGCAAATTACCGTCCTATTATATATAAAAGATAAATAAAACGTAAA
ATAAATGATGATTTATGAGATAAATTAGTGCTGCAAATTAATTTTTTATACATAAAATATTTTTTATTCGATATCAATTTTTGTGGAA
TTGTATAATGCATTTACACTATTTATCATTATTAAATAATTAATAAACATTTTTTTTATAAATAATAATGATAATAATAATAGTAAAAA
ATATTTAATTAATTAATATTTTGATTTGAATAAAGTAAAGTGTATTGTAAAAGATTTGATTTTTCTGTAGAACATAAATAAATAATAA
ATATTTAAAAATTATATTTGATTCGGTAATTCACGAAAACTATGCTCCCGTCCTTTATAAACCCTCCAACCCTTCTTTTGTTAACAC
TACACTCTTTTCTCATTCTCCTCCCTGCGGCTGTGCATTAGGGCATTTTTCTCCTTCAAGGTAAATCATTCTCCGATTTCGTTTGT
TAATCGTGTATCAGTATCATCGTTACCATCGCGATTCATAGGGTTTTCTCACATTTTAGTGTATATGTATGTGATTACGTAGGATC
ATTTGATATTAGCATGTTATTGATTATTGTTTTGCTTTCCGATTTGTTATTCGATTCCGAAATCTGCCTAGGGTTTACGTGCCTGTT
TAACTGTTATTCTGAATCCGAATAGCGTGTTAATCGATTATGCTTTTGAATCGGTTGTTTTTTTTGTAAAACTACATATGGCTATTG
ATATTTTCCAGAGATTTTTCTGTTGTGATTCTTGATGCATATCATCATAATAGACGGAAATCTATGGATTTATATAATGCTAAATAT
GATTGTGTTTGTTTTCTGATCATGTTGTGTTCTTGATCTGCTGAGATATGTTGTGGTGTATTGTCTAATTTATAGTGTAATTGTTGA
CATGGATTTTGCATATTTGATTTGTCAGAATGTTGTTGATGCTGTTTTATTTAATTTTCTTTTTTAATGCTGGTGTTTATTTTACGTG
TGTTGTTTTCTGCTTTTGATTTGTAGATTTTGTGGTTTTAAGCTTTAGTTCAA
SEQIDNO: 456
AACCAACGCATATGATCAACTAACCCTATTTGAAATACATTGTTAAAAACACTCCATTCAACCCTAAACTAAAAATGCTAAATACC
ATGACATTTTCAACATTCCTCTTGTGCGGACAACCTCAGTCTTTAACTTGAAAAATTTATTCTTTTGCCTTTCAATCTTCAAATTCC
ATCCATCAACAATTTCATCACATAACCACTTGAAAAAATTACATCCCTTTTCCATTTGATTTATGTAATTCTACAACTCCATAACTTT
TTTCTAACATTCACACTGTTCATATATGTAACTATAAGAAGAACACACCCCTCGTGGCAAAAATCTTCCCTCTTCTTGTTTTTTTTA
GCACAAATCCAAATATCCCATAATTACCGAAACACTTTAACATTTGAACATAAATTTTGAAATTATGAAAAAAATGGTAGAATATTG
ATATAAGATGTTTGTTGAGTTAGGAATTTTGCCTTGGAGTCATGCCACATCATCCTACATTAATATATTTTTTTCAAAATTTAACAA
AAAAAATTAAATTGGCTAACAAAATTAAGATTAACTAAAAATTAACTTAATACTTCGCCACTAATTAATTCTTAGTAAATTCAATTCA
AAATTTTGTAGTTGCTTAATAGAGATGGAACTCGAAATCTTTCATATGAAAAAACTCAGAAAAGCATATTCTCACGCGTACACACG
GCGTGTGACTTCAAACTCCAAGAAGGACCTAGAAGAAAATCCACGAGAGAAATACAGTGTACACACGTATTTCCTTATGAGGGT
GAAACAAACGCAACGTTCAAATAAACAAACCGAGGGCATTTCAGGAAATTCATAAGAAAACCAAGGATACTATTGGAATATAAAA
AATATTTTTCTCACTATAAAAAAACTTGAACCCTAGTTATTTCATTACCACTCTTTACTTGTTCTTAAATAGCTCAGTCACTCTGCG
GCTGAGGATTCCGAACGAGTTTTCTTCGATTTCATTTCTGCTGAGGTAAAATTCTTCGATTCATTTCTCTTTGATTCGATCTATTG
TGTTTATTTGCTGTAGTATCATATGTATGATTTTGTTAGGTTAGTTTGATTTTGGTTAATTTTGTTTAGTGTTTGAGAAATTTGTATT
TTGAACGAGTCGATTTGTGTTCGTAATCTATGATTTAATTTAGCTAATTGAGTCGATTGGTTTTCTGTATGTATAGATCTGATGAAT
TTTGGTTCAAATCAAGTAATTTTATTTTGAAAGTTATGAGCTTTTTGTGGTATGGTTGTTTAATTATGGATCTGTTAAGATTAGACT
CTGATTCAATTGATGATTGTTTAATTGATGAAAATGATATTTTTTTGATGGAGATTTGATTGTCTCTTTCTGATACTTTAAGCAGTG
GCATATTTTACCTTCAAAATTTTTGATTTTCTATTACTCGTTTTTATATGCTATAACACGAGTTGTTGATATGTTTCGAAACTTTAAA
AATTGTTTATTGTGAGTTAATTTTTCTGATTAATTGGTTAGTTTGGAGGTAAGACTCTTATTTAATCAGGGTTTGTTTTGGTTTGTT
TATGATGCAATGTTTTGATTTAACTGTTTTATTAGATCTTATTTGTGTATCTGGTTTTGAATTTGTTGCTGTTGATTTCTAGCTTATT
AGTTTTGAATTTGTTTTGAACTTCATAACATGTTCAATTGCTGTTCTTTAGTTAATATAATTTATTAGTTATGACCATGTTGAATTGC
TGTTCTTTTTGCTTATTAGTTCTGACTTTTGAATTTCTTTTTGTGAATTGCAGTTAAGATATC
SEQIDNO: 457
AGTCAAGAACGTCGTAGCTACATAATTCATACAGTCCGAACAGTCAGAGTAAAGTGAACAGTCATTGCATGCATTGTTGTATGGT
TATGGTTGGAAATGTATGATTGGTTGTTGCGTGATGGACTTATTGATTTATTTCAGATTTATGTGAGTTATCTGTTTAGATAGTTAA
TTAAATTACACTAGCTTACCCTTATTTTCCTGTATTGTCATGTTGTCCGTTCGGTCGTCCTTGTCTTTGCGATGATCACCCTGTGG
GTGTGAGCAAAAGGTGTAGAAGCTTCTCTGGAGCAGGCGCTGATAGAAGAGGATGGTCTCCGCTAAGAGTAAGACCGTTCGGA
CTTGACTTTGTTTTGGTATTTAGTATAGGACCGTTCGGTTAATACACTTCTTGTATGACCCCTTTTGAGTTGTATCAGTGTAGGTT
TCTGTAAAGTTTTTAATTTATGATTATTTTAGGATAACTGTAACTCTGTTCTATTTTTATTGTAAATATAAGTACTCTTGAATATAGTT
TGCAGCTATATTTTTGGGATGTTACAAATATTTGAATAGGTATTATTATTTTGCCACCCAATAAACTTTTACACACAGTTTACATTT
ATTATTTTATTCTTTAATTTTTCTCTTTCTTTTTAATTTTTTTTAATGACTAAAATACTCTATAAATAAATTTTCAAAGCATCTAAAATA
AAAAAGAATCACTCATTTAAATACGTAAGGTTTTGTATCATTACTAACCACCCTAATTCTTCTTTGTATCTTGTGCCGCCAGAGAA
AATAAGGCAACTAGGAATTGGCGTAAGAAGTGACATGATCTTCTACGATTTCTGCAGCACATATTCTACACATACGAATATCTAA
ACCATTAATTACACTCTATTTCTAAATTATCTTGTTGACGGTTGAGATTCGGCTATGCAATGGTTTAGACAAAACAATCGGCCGCA
GAGGATCTGGTTTGGCGTGAGGTTGGATTTTCCGTTGACGCCACGTCAAAAGAGCAGGGATACAACTAGAAATAAAGTTAAGG
GTAGTAACGCCAACAAAAATGAACAACTTGCAGTGTATAAATTCTCAGTCCCTCTTAACACAACATTCTTCTTTGCTACACTCTCT
TCTCGCGGCTAGGGTTTAAGAGCAGCTTCTTTCTTCAAGGTTATTTCTTGTTACTCACTTTCTTTTCGTTGCTTCCTTCATGTGTT
AGCCTTTATATTAGTTAATTCCTCTCCATTTACTGAAATAATTGCTGTTATTTGATTTGATTTTGGTTCTGCTTTATCTGCCTGCTAA
ATCGAAATTACCAATCCAAACAGTGCGATTATCGTGTCAATTCGAATTATTTATTTTCTTGGTCATTCCAATATATAGATGTTGGTT
TCAATCAATGTTTTTACCTAGAGCTTGGGGTCTCACGTGATGGTGTGGATATCCATTTTCATATCGTAATGTTATTTTAGTTTAAA
GCCATTATGTACTTCGTATTGTGGTGAAGTCTATATCTAGCTTTTACTCAATCTTTAATCCGTGGGGTTCCTTTTTCTTAGACATAT
CTTACCGATTCGAAAACTGTTTTATTTTGTCAACCCCGTGATGTCGTTGCTGTATGGATCATCTCATTTTATTTTCTCTTTGAGAAT
TAGGGTTAAGGATTCTGGGAAAGTACTTTTTGCAGATTTTGTTCTGTTCTTGGATGATTGACTGTTGATTTGAATGGGCAGATCTT
AATTGTTTCAATTTAGAGGACGA
SEQIDNO: 458
AACTCCTCCAAATTCGTCCAACAACCACTCCCTAATGACGATCATAGTAACAAAACCTCAATCTCTTCTTCAAAAGAGACGGTGC
ATAAAGTATCATTAATTTGCACTTGCTTCACCATCTTTGCATCTCTTCCTATAAGGATTTTGACGTGCAGCCGAGCGAACTCAACA
ACCTCCTTTGTTAGCGTGGCATTGTCAACCTCAACTAACGAACCGACAAGAGACCTAATAAATTCGAAACACTGGCTACTCCACA
ACACCAACGGGATACATCTACAGCGAACTCAAACAAATTTTTCACCTACCACAAAGTTATCGTCCCAAGGGACAAGAGACTCGA
ATATCCCATCTAACCATTCTTTATTTTCGACAACTAACTTCTCTAAGAATCCCACTTCATCACAGGATAACAACCCAAACTTCTCC
CCTAGAGACCTCAGCTTCACAGAATTAACTCCACTCAAAATAAAACTTTCCTTAATAGACTGCACATTTTTTAACTCAAAGTCTCC
CTACAAAGCAATCCTCCAACCACTTTGTAGAATCCACCACGACTTGAAAGCAGACCATGCTGGCGTCCAAGACCTTCTTAGAAG
CAGACTCGTGACCGTCCCTAACCACCTGAGCAAAGGATCTTTGTTGAGCCTTCTACCTCCAAGCCTTCCTTACATGTTCATGTTT
CCTTATCCAACTCGGGTCTGTGTGGTTCCTTCCTGCTAAACTTAGGTATATTTACCTGCAGCTTCCAAAGACCTATCCAATTAGC
GTCTAGTTTCCTTTCTAAAACGTGAGCATCCAACATCCCTTGAAATCTCACGAACCCAAAGTGCAGTTTACCATAATCTAACCTTC
TTGATATGAATACGTCGATAATCCTTCCCCACCTCTGAAAAATCTTTCACATGTCCCTCTCCATGTACTTATGAGGAAAGTTTGTG
AAGAATAACTTCACTTCCGCCATTCTTCAGCAACGTCTCTCTCTCTTACAACCCTAAACCCCAAATATTCAGCATAGAACAGAAT
GATGTTAGTTTATGTCTATGTAATAGGTTGTTTTCTTTTTATAATAAGTTTTTAATATGTTTTTTTTTTTCTATCTTCTCCTCCTTTTC
TTCTTTTTTGTTGCACAATTTTTTCTCTGTTTTTTATAGCTTATGATTGCAAAAAAGATAAGTTCAATAAAATCATAATGGATGACAA
ATTATCCATGCAAAATTAGATAGAAAATTAGCAACAATTTTAACGTGTGTTTGTGTTTAACGAACTTGTTTTGAAAAGTTAATACTC
CAAATTTGTTGGTGTGTTGACATTAATTGTTTTAATAACTATTTTGTGATGAAAAATGTGGAAGGAAGACTTCTCTACGGTGTTAC
TTGCCACCCAAGGCTAGATATTGTTTATGACCTTTTTCTAAAATAAGAATGGTCTTCTATATATGTACATAACTTGCTACCCAAGG
CCAGATATTGTTTATAAACAAATCCCTCTTAAACAACCACTTATCTCTGCTACACCTACT
SEQIDNO: 459
gagtataaaacccaaaatcaattacagttaccataatataaattttggaacctctaaaatttaactaaacaaataaggatatactaatttaaaattatttgatatttcattcatatacataaaagttg
acgtaatatatacttttaaaatacacataactatgaatagtttaattactttttattatttttcaaatttgacaaacatttaaattattatatagttataattcaatgttttgattgataaaatacactttctgttta
aagctattactggtgtaaaagtttatgtgtatatcctttttttaaggcacatgttattgataaactttcacttatttttaaaagaaacatgctgataatttacaacttgaatttgctaataagcttcaaaaa
gtgtagatttattaaaataagaatatatttgttgatcccatctcgacacttaatattgacattacaactagtcgtaattaaacaatcgaatcaattatcaataaattttttatgcaaattaactgtatcac
aattattctatatatctttaccggtatctattgatgctatttgctaccagagggttgtaccactaagagaagctactgtattgactataattaataagtaaaatacataggcatccagaatcttctgtttt
ctaacaaaagactacaaaaagggaatgcaaaaagacagagaagaacaattccactaagccgcccaaccactagtgatcaatggtagtattaattattgaacaattccaaattgtcattga
aataaggccgaacaaacattctattttaaatgttggcttcattatttatcgatctggaaaaagaaatactggcattattaattaatagtcaatgaataaaaacgtgagcacatgtaatacccatgt
acaatcatcggagtttatgagccacgaatatgaggagtctcattcatatatctctctgtcggctgtcatagtggaaaaaaaaaatgcccgttaaccatattattgccaccattgttatcatcatca
attaattttaccctccttttgttaatttgactaaatggaaaacgacgtctcaggtctcacatgtgtgtgtcttttcttttctttgtttttttaatgcaagtggatcgttgagtatgataataatatttgcttaggat
gcttatccatccaatttttatttttttttaagttcctcgtttattgtacatcaattaattaattactattgagctcatgactaagaaaataaacatgaacacaaatcataggatttttaaaatactgaaaag
agtatactaaggggttagataggtcctcgattattaatcaagcgttatccaaccactcttgtgggtccatggtcggtgcatgacaaccaagaatggctgtacattcacaatcaaatccattatatt
tatgtagtttcttgttatataaacctaagcaaaggtttatgaggcagcactaatttatatagcaagctagctaggttgaaaatagtttggttttgtagtaagtgaggtgaggtgccaaa
SEQIDNO: 460
aagtatttaggattcctataacaaaatgattttttttttttttttgattgaacaaaatgattattcttaaacaattagaaatagtatattttttagaattaatgtttttaaaacgcttgtcagtcgaatctttaa
aaataatactttaatttaattaaaatataaacattgcaagtaaaagttgaatagaattttccagttcagaggaagctaaagaattaaggttagcttacaccgttgaccctgatgcatttggcttcgc
aatttgacatattttctattcagccaagtgggaattttgcaagagacaactcatcacagatgggcgcatacataattatccattcattcattcatagcgcctaagaatttctctccatgtagttgggt
caaaatagcacttagactgattgggactttttcttttcttatttttgtcaacctcctcaaatttgactgtagtaaaagggccatgcacattactgacttatcttatagtttctactttcttccttatatttaatta
gaaaaaaattctaagaatggaatttctcataaaaataatgtgtgggaaatgaaagcccccttgccgtttgtttaggtattagactgcgttggcttttccataactgtacagctcttgcacacttcttgt
ttgcttgccccttctgagacacaaggggacaaaacaaataattaaagatataaacttcaatctacattacattctcttgggaagggaggaataggatattaggatatcaatagtatattcacatt
tggatttggatgagctctagattcttattaggcaattctagagttatataatataatattgaatattaaatttactttatttttgttgtgatttatttcaattttactttgtcactgtctggtttttaaaagtgtcaaa
ctttaaagtttattttactgttaaattttattcaaaatctgtatacagaaattcttgtatccaatttaaatttttatgtaaatattttatgttatgggaacataagatatgaagcatgtaaaaaaaatcacga
ttgatttcgtgatatataaagagcaattagaaaccacaaaggaaaaaagtgttgtcggaccaatgctcaacaagaaacagtgatagtgtgatacaacaaaaaattgaaccgggctttccttt
aagattttaggcataccgttaatgtctttttttgtttttgacgttgatgtctgtttggtaaaattgaagaaagagctgaatcaggttgggaacttgggacaaacctgtaatctatctaatgcattatatgt
acttgcatgtggccatcataaactccaagcacttggggagaaggagaaaatagaaaaataaataaataacaaagtcaactgatcaaaactaaattaaattttgaaaaccgactccatgtc
tccatatgtacaatcatcacccggtgctgcttctcaacaggctggcctttcaaactgtttaagggatatatctctaccacttgcttgcttctgctattatttttaattttatgctgtagttttggaatttgagc
cttttgattgtcttttggaaaaaaaaagttaaatataagtaaaaaggaaacgttttgtatgaaagtgtgatgtaaatttctttatatcatcgttaatgtaaatatgtcttatttttagttccctcaatttttca
acattttcatacttgtcatttctcaaaattcaatttgtttttatttccctatatttttatggaattgggctttatataaaggctttacattcagtgcatccccactcagaatcttagaatatcaatcactccgatt
cctgttggttgttctctcaagtctcaactcccaccccccctctttgacttttctatcagaagagcttgttggagttgaaaact
SEQIDNO: 461
gaattcacaaacacttttgacagcgttaatggaagatatttctttaggttcgagattgaaacttgataaaggatggttacctagttgtctttcctgctgtgccacgtacgaaaagattttgggtattgt
gggtattggatccatcagcaacacatgagaatgtatattgtcatactgctcattcaaccctcgcaggaactgcatcgtgcgatcttcttgctttcgttgagctatggtggtgagtaccaagcaagt
acacttgacagtgcatggcatacggggtttggcctgaagttttcaatctcatcccatatgacccgcagcttcgtgaagtattctgttactgagagagaaccttgcttaatggatgaagcttcttgtt
ggagttcagaaactctcagaaggtccccttgcgcgcacctagacttcagatcattccaaatctcttccgctttgtccatccacaagacactttgtctaatggggatggatactgaatgaaccaa
ccaagacactaccatgttattacagcgactccatgccccgtgagttctatcatatttcaatggctcgggtgcttttccatttacaaactctactttgttcttggcgcttaatgccgtcatcatggaccta
ctccatgaatgataattgctagaatctaaaggaggtgaaaccagtgcaacagctaggttctcacttgggtgaaggtaaagataactatctgtgcttagcattgactcgttcatggtgagaaag
cagatgagaggaaaacacgggagtgtaaaagagagaggtgacacagcagaactcttcttcaagaagagctctgataccatgttaataaaggtattgtgccatagagagagaaagata
agcgtgaatcacgtgtaaaatgaccaaatactgcatgccttgtattgatgcatttattatgtatttatatctctgcagagtttgcagaggagagagaaaacgttggtaattttagtcagttacactcg
agaaccaattctaactaactaacaaaaaactaattgatgaaacgaacttatgttaagataacaagcttccgctaagatgggcattccttctcttaaaaatgtatttcttttgtatatcatgaccgaa
tccgttaattaaccttgttagtgtattgtgaaaggtcaaaattacccttcatacttcaatccctcatttcgctactctccaacatgttctggaaatcccaaattaaaaacctcaaaatgtgttctgctag
ggtttgagttctctctcaggtttgatgcccatcattttttgtgtgttaggatttcaacggccaccatatgcgtcattttgggttaaaagtagatttctctactaatttttttagtgttagacaaattgtgatttgc
gtcaaattgtgaaacttttaatataaaaacaaatgaaacccatatctatgatcaataactcacaaatgcatgaaaaaatcaaacaatagttgttgaactgaaaaattattgataatttagaatttt
ttatataagactaaactggttataaataaagtttgatcattatgaaatatgaacttataactttctaattatotttgaagtatctaatcatgtaacattattataaaatattcttatttttactttatcattatca
ataaattaactagttaattattccacttacttggttgaagactactttgaacctatctttttactctcttctcaatgttttogtttaaacaattactgagtataacttaataataaatacataatacatataaa
aagatcgatgaaacaccctagtaattaattataaaataatttagttttaatacggcataacaaatttacagtctcaattaatataaaatgttataaaaatcatcaatattaaaactgtttcatttaatt
aaattcattataaaatgtttgccggtcaaatgtaatagacacagacacggagaatataatacaaaagtttcattactctctctctctctctgttgaataaccctccacaaccaccttattcaatcttt
aaatagcttattgattaatgtcttatagtc
SEQIDNO: 462
aaaGAATTCACCATCCCTATGTTTGACAC
SEQIDNO: 463
aaaCTCGAGTTGAGTTAAACTGAAACCAATTC
SEQIDNO: 464
aaaGAATTCAGCTTTTGGAGAGTTTCATATC
SEQIDNO: 465
aaaCTCGAGTTGAGTTAAACTGAAACCAA
SEQIDNO: 466
aaaGAATTCTGACAAGGTCAAGAAGGAGT
SEQIDNO: 467
aaaCTCGAGTAGTCCTTGAGCTTGAAATC
SEQIDNO: 468
aaaGAATTCACATGGTACACACCTCTGTC
SEQIDNO: 469
aaaCTCGAGTTGAACTAAAGCTTAAAACCAC
SEQIDNO: 470
aaaGAATTCAACCAACGCATATGATCAAC
SEQIDNO: 471
aaaCTCGAGATATCTTAACTGCAATTCACA
SEQIDNO: 472
aaaGAATTCAGTCAAGAACGTCGTAGCTA
SEQIDNO: 473
aaaCTCGAGTCGTCCTCTAAATTGAAACAA
SEQIDNO: 474
aaaGAATTCAACTCCTCCAAATTCGTCCA
SEQIDNO: 475
aaaCTCGAGTAGGTGTAGCAGAGATAAG
SEQIDNO: 476
aaacGAGTATAAAACCCAAAATC
SEQIDNO: 477
aaaCTCGAGTTTGGCACCTCACCTCAC
SEQIDNO: 478
ATGAATTCAAGTATTTAGGATTCCTATAACAAAATG
SEQIDNO: 479
AACTCGAGTTTTCAACTCCAACAAGCTCTTC
SEQIDNO: 480
TGCGTCCACAGAATTCACAAACACT
SEQIDNO: 481
AAGTCGACTATAAGACATTAATCAATAAGCT
SEQIDNO: 482
AATCTAGACGATCTCTCTCAAGGGACCTTGCT
SEQIDNO: 483
AATCTAGATATTGCTTCTTATACAGATCTAAGGCA

### REFERENCES

U.S. Pat. No. 5,254,799A, 1990
U.S. Pat. No. 5,561,236A, 1990
U.S. Pat. No. 5,495,071A, 1993
U.S. Pat. No. 5,627,061A, 1995
U.S. Pat. No. 5,914,318A, 1997
Castiglioni, P., Warner, D., Bensen, R. J., Anstrom, D. C., Harrison, J., Stoecker, M., Abad, M., Kumar, G., Salvador, S., D'Ordine, R., Navarro, S., Back, S., Fernandes, M., Targolli, J., Dasgupta, S., Bonin, C., Luethy, M. H., & Heard, J. E. (2008) Bacterial RNA chaperones confer abiotic stress tolerance in plants and improved grain yield in maize under water-limited conditions. Plant Physiol. 147(2):446-455.
Domingos P, Prado A M, Wong A, Gehring C, Feijo J A. (2015) Nitric oxide: a multitasked signaling gas in plants. Mol Plant. 8(4):506-520.
Gaudet P., Livstone M. S., Lewis S. E., Thomas P. D. (2011) Phylogenetic-based propagation of functional annotations within the Gene Ontology consortium. Brief. Bioinformatics 12:449-462.
Karaba, A.; Dixit, S. A.; Greco, Raffaella; Aharoni, A.; Trijatmiko, K. R.; Marsch-Martinez, N.; Krishnan, A.; Nataraja, K. N.; Udayakumar, M.; Pereira, A. B. (2007) Improvement of water use efficiency in rice by expression of HARDY, an Arabidopsis drought and salt tolerance gene. Proceedings of the National Academy of Sciences of the United States of America 104 (39): 15270-15275.
Kasuga M, Miura S, Shinozaki K, Yamaguchi-Shinozaki K. (2004) A combination of the Arabidopsis DREB1A gene and stress-inducible rd29A promoter improved drought- and low-temperature stress tolerance in tobacco by gene transfer. Plant Cell Physiol. 45(3):346-350.
Mega R, Abe F, Kim J S, Tsuboi Y, Tanaka K, Kobayashi H, Sakata Y, Hanada K, Tsujimoto H, Kikuchi J, Cutler S R, Okamoto M (2019) Tuning water-use efficiency and drought tolerance in wheat using abscisic acid receptors. Nature Plants 5(2):153-159.
Nelson D E, Repetti P P, Adams T R, Creelman R A, Wu J, Warner D C, Anstrom D C, Bensen R J, Castiglioni P P, Donnarummo M G, Hinchey B S, Kumimoto R W, Maszle D R, Canales R D, Krolikowski K A, Dotson S B, Gutterson N, Ratcliffe O J, Heard J E. (2007) Plant nuclear factor Y (NF-Y) B subunits confer drought tolerance and lead to improved corn yields on water-limited acres. PNAS 104 (42) 16450-16455.
Takahashi F, Kuromori T, Sato H, Shinozaki K. (2018) Regulatory Gene Networks in Drought Stress Responses and Resistance in Plants. Adv Exp Med Biol. 1081:189-214.
Vogel J P., Garvin D F., Leong O M. & Hayden D M. (2006) Agrobacterium-mediated transformation and inbred line development in the model grass Brachypodium distachyon. Plant Cell, Tissue and Organ Culture 84: 199-211.
Weigel D, Ahn J H, Blezquez M A, Borevitz J O, Christensen S K, Fankhauser C, Ferrendiz C, Kardailsky I, Malancharuvil E J, Neff M M, Nguyen J T, Sato S, Wang Z Y, Xia Y, Dixon R A, Harrison M J, Lamb C J, Yanofsky M F, Chory J. (2000) Activation tagging in Arabidopsis. Plant Physiol. 122(4):1003-1013.
Yang S, Vanderbeld B, Wan J, Huang Y. (2010) Narrowing down the target: towards successful genetic engineering of drought-tolerant crops. Mol. Plant 3(3):469-490.
Yang Z, Liu J, Poree F, Schaeufele R, Helmke H, Frackenpohl J, Lehr S, von Koskull-Döring P, Christmann A, Schnyder H, Schmidhalter U, Grill E. (2019) Abscisic Acid Receptors and Coreceptors Modulate Plant Water Use Efficiency and Water Productivity. Plant Physiology 180(2):1066-1080.
Yoo, C. Y., Pence, H. E., Jin, J. B., Miura, K., Gosney, M. J., Hasegawa, P. W. and Mickelbart, M. V. (2010) The Arabidopsis GTL1 transcription factor regulates water use efficiency and drought tolerance by modulating stomatal density via transrepression of SDD1. Plant Cell, 22, 4128-4141.
Zhu J. K. (2016) Abiotic Stress Signaling and Responses in Plants. Cell 167(2):313-324.

1. A method of increasing tolerance to water and/or nutrient deficiency in a plant, comprising: modifying expression or activity of AtExo970, or a homolog or ortholog thereof.

2. The method of claim 1, wherein said method comprises a) introducing a nucleic acid to a plant, a plant tissue culture or a plant cell to obtain a modified plant, a modified plant tissue culture or a modified plant cell, wherein the nucleic acid encodes the AtExo970, homolog or ortholog thereof, b) growing the modified plant or regenerating a plant from the modified plant tissue culture or the modified plant cell; and c) selecting a plant having increased tolerance to water and/or nutrient deficiency relative to a wild type plant.

3. The method of claim 2, wherein the nucleic acid comprises a sequence having at least 50%, 55%, 60%, 65%, 70%, 75%, 80%, 85%, 90%, 95%, 96%, 97%, 98%, 99% or 100% identity to selected from the group consisting of SEQ ID NOs: 122, 123, 126, 127, 128, 129, 131, 132, 134, 135, 137, 138, 140, 141, 143, 144, 146, 147, 149, 150, 152, 153, 155, 156, 158, 159, 161, 162, 164, 165, 167, 168, 170, 171, 173, 174, 175, 177, 178, 180, 181, 182, 184, 185, 187, 188, 190, 191, 192, 194, 195, 196, 198, 200, 201, 203, 205, 206, 208, 209, 212, 213, 215, 216, 218, 219, 221, 222, 224, 225, 227, 228, 230, 231, 233, 234, 236, 237, 239, 240, 242, 243, 245, 246, 248, 249, 251, 252, 254, 255, 257, 258, 260, 261, 263, 264, 266, 267, 269, 270, 272, 273, 275, 276, 278, 279, 281, 282, 284, 285, 287, 288, 290, 291, 293, 294, 296, 297, 299, 300, 302, 303, 305, 306, 308, 309, 311, 312, 314, 315, 317, 318, 320, 321, 323, 324, 326, 327, 329, 330, 332, 333, 335, 336, 338, 339, 341, 342, 343, 344, 345, 347, 348, 350, 351, 353, 354, 356, 357, 359, 360, 362, 363, 365, 366, 368, 370, 371, 373, 374, 376, 377, 379, 380, 382, 383, 385, 386, 388, 389, 391, 392, 394, 395, 397, 398, 400, 401, 403, 404, 406, 407, 409, and 410; or
wherein the nucleic acid encodes a polypeptide comprising a sequence at least 50%, 55%, 60%, 65%, 70%, 75%, 80%, 85%, 90%, 95%, 96%, 97%, 98%, 99% or 100% identity to a sequence selected from the group consisting of SEQ ID NOs: 124, 125, 130, 133, 136, 139, 142, 145, 148, 151, 154, 157, 160, 163, 166, 169, 172, 176, 179, 183, 186, 189, 193, 197, 199, 202, 204, 207, 211, 214, 217, 220, 223, 226, 229, 232, 235, 238, 241, 244, 247, 250, 253, 256, 259, 262, 265, 268, 269, 270, 271, 272, 273, 274, 275, 276, 277, 278, 279, 280, 283, 286, 289, 292, 295, 298, 301, 304, 307, 310, 313, 316, 319, 322,325, 328, 331, 334, 337, 340, 343, 346, 349, 352, 355, 358, 361, 364, 367, 369, 372, 375, 378, 381, 384, 387, 390, 393, 396, 399, 402, 405, and 408.

4. (canceled)

5. The method of claim 1, wherein said method comprises_(i mutating the native promoter or gene encoding said AtExo970, homolog or ortholog thereof; (ii) replacing the native promoter or the gene encoding said AtExo970, homolog or ortholog thereof; or (iii) replacing a portion of said promoter or said gene.

6. The method of claim 5, wherein said mutating comprises site-directed, random mutagenesis, CRISPR mediated, Transcription activator-like effector nucleases (TALEN) mediated or zinc finger nuclease mediated.

7. (canceled)

8. (canceled)

9. (canceled)

10. The method of claim 1, wherein said method comprises a) introducing one or more nucleic acids for CRISPR mediated replacement of the native promoter of the gene for AtExo970, homolog or ortholog to a plant, a plant tissue culture or a plant cell to obtain a modified plant, a modified plant tissue culture or a modified plant cell; b) growing the modified plant or regenerating a plant from the modified plant tissue culture or the modified plant cell; and c) selecting a plant having increased tolerance to water and/or nutrient deficiency relative to a wild type plant.

11. The method of claim 10, wherein the one or more nucleic acids for CRISPR mediated replacement of the native promoter comprise: one or more nucleic acids capable of expressing a CAS endonuclease, and a CAS endonuclease guide RNA targeting the native promoter of the gene encoding AtExo970, homolog or ortholog thereof and a homology-directed repair (HDR) template containing the new promoter.

12. The method of claim 1, further comprising introducing one or more nucleic acids encoding one or more herbicide resistance proteins and/or more or more pest resistance proteins.

13. A plant produced by the method of claim 1.

14. A seed produced by the plant of claim 13.

15. A method of increasing plant productivity, comprising: modifying expression or activity of AtExo970, or a homolog or ortholog thereof.

16. The method of claim 15, wherein said method comprises (i) mutating the native promoter or gene encoding said AtExo970, homolog or ortholog thereof; (ii) replacing the native promoter or the gene encoding said AtExo970, homolog or ortholog thereof; or (iii) or replacing a portion of said promoter or said gene.

17. The method of claim 16, wherein said mutating comprises site-directed, random mutagenesis, CRISPR mediated, Transcription activator-like effector nucleases (TALEN) mediated or zinc finger nuclease mediated.

18. (canceled)

19. (canceled)

20. (canceled)

21. The method of claim 15, wherein said method comprises a) introducing a nucleic acid to a plant, a plant tissue culture or a plant cell to obtain a modified plant, a modified plant tissue culture or a modified plant cell, wherein the nucleic acid encodes the AtExo970, homolog or ortholog thereof, b) growing the modified plant or regenerating a plant from the modified plant tissue culture or the modified plant cell; and c) selecting a plant having increased plant productivity relative to a wild type plant.

22. The method of claim 21, wherein the nucleic acid comprises a sequence having at least 50%, 55%, 60%, 65%, 70%, 75%, 80%, 85%, 90%, 95%, 96%, 97%, 98%, 99% or 100% identity to a sequence selected from the group consisting of SEQ ID NOs: 122, 123, 126, 127, 128, 129, 131, 132, 134, 135, 137, 138, 140, 141, 143, 144, 146, 147, 149, 150, 152, 153, 155, 156, 158, 159, 161, 162, 164, 165, 167, 168, 170, 171, 173, 174, 175, 177, 178, 180, 181, 182, 184, 185, 187, 188, 190, 191, 192, 194, 195, 196, 198, 200, 201, 203, 205, 206, 208, 209, 212, 213, 215, 216, 218, 219, 221, 222, 224, 225, 227, 228, 230, 231, 233, 234, 236, 237, 239, 240, 242, 243, 245, 246, 248, 249, 251, 252, 254, 255, 257, 258, 260, 261, 263, 264, 266, 267, 269, 270, 272, 273, 275, 276, 278, 279, 281, 282, 284, 285, 287, 288, 290, 291, 293, 294, 296, 297, 299, 300, 302, 303, 305, 306, 308, 309, 311, 312, 314, 315, 317, 318, 320, 321, 323, 324, 326, 327, 329, 330, 332, 333, 335, 336, 338, 339, 341, 342, 343, 344, 345, 347, 348, 350, 351, 353, 354, 356, 357, 359, 360, 362, 363, 365, 366, 368, 370, 371, 373, 374, 376, 377, 379, 380, 382, 383, 385, 386, 388, 389, 391, 392, 394, 395, 397, 398, 400, 401, 403, 404, 406, 407, 409, and 410; or
wherein the nucleic acid encoding a polypeptide comprising a sequence at least 50%, 55%, 60%, 65%, 70%, 75%, 80%, 85%, 90%, 95%, 96%, 97%, 98%, 99% or 100% identity to a sequence selected from the group consisting of SEQ ID NOs: 124, 125, 130, 133, 136, 139, 142, 145, 148, 151, 154, 157, 160, 163, 166, 169, 172, 176, 179, 183, 186, 189, 193, 197, 199, 202, 204, 207, 211, 214, 217, 220, 223, 226, 229, 232, 235, 238, 241, 244, 247, 250, 253, 256, 259, 262, 265, 268, 269, 270, 271, 272, 273, 274, 275, 276, 277, 278, 279, 280, 283, 286, 289, 292, 295, 298, 301, 304, 307, 310, 313, 316, 319, 322,325, 328, 331, 334, 337, 340, 343, 346, 349, 352, 355, 358, 361, 364, 367, 369, 372, 375, 378, 381, 384, 387, 390, 393, 396, 399, 402, 405, and 408.

23. (canceled)

24. The method of claim 15, wherein said method comprises a) introducing one or more nucleic acids for CRISPR mediated replacement of the native promoter of the gene for AtExo970, homolog or ortholog to a plant, a plant tissue culture or a plant cell to obtain a modified plant, a modified plant tissue culture or a modified plant cell; b) growing the modified plant or regenerating a plant from the modified plant tissue culture or the modified plant cell; and c) selecting a plant having increased tolerance to water and/or nutrient deficiency relative to a wild type plant.

25. The method of claim 24, wherein the one or more nucleic acids for CRISPR mediated replacement of the native promoter comprise: one or more nucleic acids capable of expressing a CAS endonuclease, and a CAS endonuclease guide RNA targeting the native promoter of the gene encoding AtExo970, homolog or ortholog thereof and a homology-directed repair (HDR) template containing the new promoter.

26. The method of claim 15, further comprising introducing one or more nucleic acids encoding one or more herbicide resistance proteins and/or more or more pest resistance proteins.

27. A plant produced by the method of claim 15.

28. A seed produced by the plant of claim 27.
